(19) United States
(12) Patent Application Publication
(10) Pub. No.: US 2026/0250301 A1
(43) Pub. Date: Aug. 27, 2026

(54) KRAS G12D INHIBITORS AND USES THEREOF

(71) Applicant: SUZHOU ZANRONG PHARMA LIMITED, Suzhou, Jiangsu (CN)

(72) Inventors: Zheng WANG, Shanghai (CN); Ding ZHOU, Shanghai (CN); Ziqiang CHENG, Shanghai (CN)

(21) Appl. No.: 19/148,326

(22) PCT Filed: Jan. 17, 2024
(86) PCT No.: PCT/CN2024/072745

§ 371 Date: Jul. 16, 2025

(30) Foreign Application Priority Data
Jan. 18, 2023 (WO) PCT/CN2023/072979

(51) Int. Cl. C07D 498/22; A61K 31/553; A61P 35/00

(52) U.S. Cl. CPC C07D 498/22; A61K 31/553; A61P 35/00

(57) ABSTRACT

Provided are compounds for the inhibition of KRAS mutans, in particular KRAS G12D, as well as pharmaceutical compositions comprising these compounds and methods of treatment by administration of these compounds or the pharmaceutical compositions.

### FIELD OF THE DISCLOSURE

The present disclosure generally relates to useful compounds for the inhibition of KRAS mutants, in particular KRAS G12D, as well as pharmaceutical compositions comprising these compounds and methods of treatment by administration of these compounds or the pharmaceutical compositions.

### BACKGROUND OF THE DISCLOSURE

RAS is one of the most well-known proto-oncogenes. Its gain-of-function mutations occur in approximately 30% of all human cancers. As the most frequently mutated RAS isoform, KRAS (Kirsten-rat sarcoma viral oncogene homolog) is intensively studied in the past years. KRAS and the highly related NRAS and HRAS GTPases hydrolyze guanosine triphosphate (GTP) to guanosine diphosphate (GDP). They control diverse cellular functions by cycling between an active, GTP-bound and an inactive, GDP-bound conformation (Hobbs, G. A., et al. J. Cell Sci. 129, 1287-1292. (2016)).

KRAS is a prominent oncogene that has been proven to drive tumorigenesis (G G Jinesh, et al. Oncogene volume 37, pages 839-846 (2018)). KRAS also modulates numerous genetic regulatory mechanisms and forms a large tumorigenesis network. KRAS gene encodes a 21 kDa protein, called KRAS, part of the RAS/MAPK pathway. The KRAS protein is a GTPase, which means it binds to guanine nucleotides GDP and guanosine-triphosphate (GTP) with high affinity and can hydrolyze GTP to GDP (Dhirendra K. Simanshu, et al. Cell. 2017 Jun. 29; 170(1): 17-33). GDP/GTP cycling is tightly regulated by a diverse family of multi-domain proteins: guanine nucleotide exchange-factors (GEFs) and GTPase-activating proteins (GAPs). GEFs stimulate the dissociation of GDP and subsequent association of GTP, activating RAS proteins, while GAPs act to accelerate intrinsic GTP hydrolysis, converting RAS to its inactive state (Dhirendra K. Simanshu, et al. Cell. 2017 Jun. 29; 170(1): 17-33). The GTP bound form of KRAS is considered the active form, and downstream signaling effectors specifically bind to the GTP-bound form of KRAS. The KRAS protein is turned off (inactivated) when the protein is bound to GDP and does not relay signals to the cell's nucleus.

The cancer-promoting KRAS mutations most commonly occur at codon 12, 13, or 61 (Jozsef Timar, et al. Cancer and Metastasis Reviews volume 39, pages 1029-1038 (2020)). Among these mutation sites, G12 is the most frequently mutated residue (89%) and it most often mutates to aspartate (G12D, 36%) followed by valine (G12V, 23%) and cysteine (G12C, 14%). G12 is located at the protein active site, which consists of a phosphate binding loop (P-loop, residues 10-17) and two switch regions (Switch-I (SI), residues 25-40, and Switch-II (SII), residues 60-74) (Prior, I. A., et al. Cancer Res 72, 2457-2467, (2012)). The residues in the active site bind to the phosphate groups of GTP and are responsible for the GTPase function of KRAS. The switch regions SI and SII are additionally responsible for controlling binding to effector and regulator proteins. The mutation of glycine at position 12 to aspartate (G12D) in the P-loop leads to impair GTP hydrolysis and freeze KRAS in its active (GTP-bound) state, which causes uncontrollable cellular growth and evasion of apoptotic signals (Malumbres, M. & Barbacid, M. Nat Rev Cancer 3, 459-465, (2003)). The G12D mutation causes a shift in the population of local conformational states of KRAS, especially in Switch-II (SII) and a3-helix regions, in favor of a conformation that is associated with a catalytically impaired state through structural changes; it also causes SII motions to anti-correlate with other regions (Sezen Vatansever, et al. Sci Rep. 2019 Aug. 13; 9(1):11730).

KRAS mutations are present in up to 25% of cancers, the oncogenic variants have different prevalence rates in different cancers. In pancreatic ductal adenocarcinoma cases, the most common KRAS alteration is the G12D substitution. The G12D variant is also the focus of drug discovery efforts by Mirati, which plans to bring its lead compound, MRTX1133 to clinical trials. Based on epidemiology data reported in Globocan 2022 (accessed November 2019) and frequencies by mutation, KRAS G12D mutation is present in an estimated around 36% of Pancreatic cancer, in 4% colorectal cancer, in around 6% endometrial cancer and in around 4% NSCLC. This significant patient population with high unmet need.

Apart from KRAS G12D mutation, other KRAS mutation, such as KRAS (G12C), KRAS (G12V), KRAS (G12A), KRAS (G12S) or KRAS (G12R), also influences the function of KRAS and the occurrence, development of tumors or resistance to target therapy. Other KRAS mutations or secondary mutations of KRAS that disrupt covalent or potentially noncovalent drug binding can be used to illustrate clinical resistance to KRAS-mutant targeting therapy (Awad M M, et al. N Engl J Med. 2021; 384(25):2382-93). KRAS gene amplification and overexpression are also relevant for tumor progression (E Birkeland, et al. Br. J Cancer. 2012 Dec. 4; 107(12):1997-2004). The publication also suggested wild type KRAS inhibition could also be a viable therapeutic strategy to treat KRAS wild type dependent cancer (Lisa Maria Mustachio, et al. Cancers (Basel). 2021 March; 13(6): 1204).

Therefore, there is still unmet need to develop new compounds efficacious in the treatment of cancers medicated by KRAS, especially for KRAS mutated in position 12, for example G12D, or 13 and/or in wild-type amplified KRAS mediated cancer.

### SUMMARY OF THE DISCLOSURE

Disclosed herein are novel compounds that are capable of inhibiting KRAS G12D proteins. As a result, the compounds of the present disclosure are useful in the treatment of KRAS G12D-associated diseases such as cancers.

In one aspect, the present disclosure provides a compound having Formula (I) or Formula (I′):

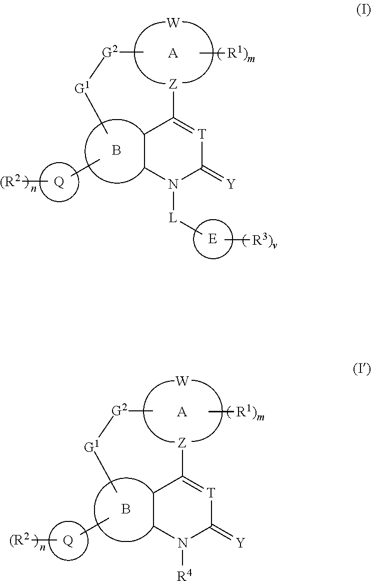

or a pharmaceutically acceptable salt thereof,
wherein
   Y is O or S;
   T is N or C(R^{T});
   R^{T} is selected from hydrogen, halogen, hydroxyl, cyano, alkyl, heteroalkyl, haloalkyl or —OR^{b};
   Ring A is heterocyclyl or heteroaryl,
   Z is C(R^{e}) or N;
   R^{e} is absent or hydrogen;
   W is N(R^{1W}) or C(R^{2W})(R^{3W});
   R^{1W} is absent, hydrogen, hydroxyl, alkyl, cycloalkyl, heterocyclyl or heteroalkyl;
   R^{2W} is absent, hydrogen, alkyl or cycloalkyl;
   R^{3W} is hydrogen, hydroxyl, —N(R^{a})₂, alkyl or heteroalkyl;
   each R¹ is independently selected from the group consisting of hydrogen, oxo, hydroxyl, halogen, cyano, alkyl, alkenyl, alkynyl, alkylalkoxy, heteroalkyl, heteroaryl, —C(O)R*, —C(O)OR*, —C(O)N(R^{a})₂ and —N(R^{a})₂, wherein the alkyl, alkenyl, alkynyl, alkylalkoxy, heteroalkyl and heteroaryl are optionally substituted with one or more groups independently selected from deuterium, cyano, hydroxyl, halogen, —OR^{b}, or —N(R^{b})₂, provided that if R¹ is present, R¹ is not connected to W;
   each R^{a} and R^{b} is independently hydrogen, alkyl, alkenyl or alkynyl, wherein the alkyl and alkenyl are optionally substituted with one or more deuterium;
   R* is selected from hydrogen, alkyl, alkylaryl or aryl;
   Ring B is selected from cycloalkyl, heterocyclyl, aryl, or heteroaryl, each of which is optionally substituted with one or more R′;
   each R′ is independently selected from the group consisting of oxo, hydroxyl, halogen, cyano, amino, alkyl, alkenyl, alkynyl, heteroalkyl, heteroalkenyl, heteroalkynyl, heteroaryl, wherein alkyl, alkenyl, alkynyl, heteroalkyl, heteroalkenyl, heteroalkynyl and heteroaryl are optionally substituted with one or more group independently consisting of deuterium, hydroxyl, halogen, cyano, —OR^{a}, —N(R^{a})₂, and heteroaryl;
   Ring Q is selected from cycloalkyl, heterocyclyl, aryl or heteroaryl;
   each R² is independently selected from the group consisting of hydrogen, oxo, hydroxyl, halogen, cyano, amino, nitro, alkyl, alkenyl, alkynyl, alkoxy, haloalkyl, heteroalkyl, heteroalkenyl, heteroalkynyl, cycloalkyl, heterocyclyl, aryl, heteroaryl and —C(O)R*, wherein alkyl, alkenyl, alkynyl, alkoxy, heteroalkyl, heteroalkenyl, heteroalkynyl, cycloalkyl, heterocyclyl, aryl, and heteroaryl are optionally substituted with one or more group independently consisting of deuterium, hydroxyl, halogen, cyano, amino, nitro, alkyl, alkoxy, haloalkyl, hydroxyalkyl, cycloalkyl, heterocyclyl, aryl and heteroaryl;
   G¹ is a bond, —[C(R^{d})₂]ᵤ—, —O—, —S(O)ₚ—, —S—S—, —N(R^{c})—, or —C(R^{d})═C(R^{d})—;
   G² is a bond, —[C(R^{d})₂]ᵤ—, —N(R^{c})—, —C(O)— or —C(O)C(R^{d})₂—;
   R^{c} is selected from the group consisting of hydrogen, alkyl, haloalkyl, hydroxyalkyl, heteroalkyl, cycloalkyl and heterocyclyl, wherein the alkyl, haloalkyl, hydroxyalkyl, heteroalkyl, cycloalkyl and heterocyclyl are optionally substituted with one or more deuterium;
   each R^{d} is independently selected from the group consisting of hydrogen, hydroxyl, halogen, cyano, amino, nitro, alkyl, alkenyl, alkynyl, alkoxy, heteroalkyl, heteroalkenyl, heteroalkynyl, cycloalkyl, and heterocyclyl, wherein alkyl, alkenyl, alkynyl, alkoxy, heteroalkyl, heteroalkenyl, heteroalkynyl, cycloalkyl, and heterocyclyl are optionally substituted with one or more group independently consisting of hydroxyl, halogen, cyano, amino, nitro, alkyl, alkoxy, haloalkyl, and hydroxyalkyl; or
   two R^{d} together with the carbon atom to which they are both attached form a cycloalkyl or heterocyclyl, wherein cycloalkyl and heterocyclyl are optionally substituted with cyano, halogen, hydroxyl, amino, nitro, alkoxy, haloalkyl, hydroxyalkyl and alkyl;
   L is selected from alkyl, alkenyl, alkynyl, alkylcycloalkylalkyl, or alkylheterocyclylalkyl, each of which is optionally substituted with one or more R^{f};
   each R^{f} is independently selected from deuterium, alkyl, hydroxyl, halogen, cyano or amino, wherein the alkyl and amino are optionally substituted with one or more groups independently selected from deuterium, hydroxyl and alkyl;
   Ring E is selected from cycloalkyl, heterocyclyl, aryl or heteroaryl;
   each R³ is independently selected from the group consisting of hydrogen, deuterium, oxo, hydroxyl, halogen, alkyl, alkoxy, haloalkyl, hydroxyalkyl, cycloalkyl, heterocyclyl, —N(R^{g})₂ and ═C(R^{g})₂, wherein the alkyl, alkoxy, haloalkyl, hydroxyalkyl, cycloalkyl, heterocyclyl are optionally substituted with one or more groups selected from deuterium, cycloalkyl or heterocyclyl;
   each R^{g} is independently hydrogen, alkyl, alkenyl or alkynyl;
   each R″ is independently selected from hydrogen, hydroxyl, halogen, cyano, alkyl, cycloalkyl, hydroxyalkyl, haloalkyl or heterocyclyl;
   R⁴ is alkyl optionally substituted with one or more group independently selected from deuterium, halogen, hydroxyl, hydroxyalkyl, alkyl, alkoxy, —N(R^{h})₂ or -alkyl-N(R^{h})₂;
   each R^{h} is independently hydrogen, alkyl, alkenyl or alkynyl;
   m is an integer from 0 to 6;
   n is an integer from 0 to 5;
   p is an integer from 0 to 2;
   u is an integer from 0 to 4; and
   v is an integer from 0 to 4.

In another aspect, the present disclosure provides a compound having a formula selected from:

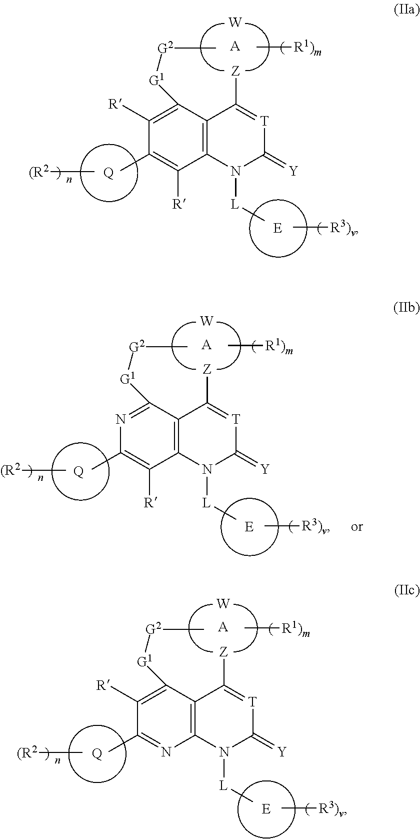

or a pharmaceutically acceptable salt thereof.

In a further aspect, the present disclosure provides a compound having a formula selected from Formula (I), (IIa), (IIb) or (IIc), wherein L is

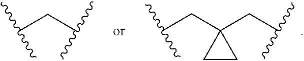

In another aspect, the present disclosure provides a pharmaceutical composition comprising the compound of the present disclosure or a pharmaceutically acceptable salt thereof, and a pharmaceutically acceptable excipient.

In a further aspect, the present disclosure provides a method for inhibiting KRas G12D activity in a subject in need thereof, comprising administering an effective amount of a compound of the present disclosure or a pharmaceutically acceptable salt thereof or the pharmaceutical composition of the present disclosure to the subject.

In a further aspect, the present disclosure provides a method for treating a KRas G12D-associated cancer comprising administering an effective amount of a compound of the present disclosure or a pharmaceutically acceptable salt thereof or the pharmaceutical composition of the present disclosure to a subject in need thereof.

In a further aspect, the present disclosure provides a method for treating cancer in a subject in need thereof, the method comprising:
(a) acquiring the knowledge that the cancer is associated with a KRas G12D mutation; and
(b) administering to the subject an effective amount of a compound of the present disclosure or a pharmaceutically acceptable salt thereof or the pharmaceutical composition of the present disclosure.

In another aspect, the present disclosure provides use of the compound of the present disclosure or a pharmaceutically acceptable salt thereof or the pharmaceutical composition of the present disclosure in the manufacture of a medicament for treating cancer.

In another aspect, the present disclosure provides a compound of present disclosure or a pharmaceutically acceptable salt thereof or the pharmaceutical composition of the present disclosure, for use in the treatment of cancer.

### DETAILED DESCRIPTION OF THE DISCLOSURE

Reference will now be made in detail to certain embodiments of the present disclosure, examples of which are illustrated in the accompanying structures and formulas. While the present disclosure will be described in conjunction with the enumerated embodiments, it will be understood that they are not intended to limit the present disclosure to those embodiments. On the contrary, the present disclosure is intended to cover all alternatives, modifications, and equivalents, which may be included within the scope of the present disclosure as defined by the claims. One skilled in the art will recognize many methods and materials similar or equivalent to those described herein, which could be used in the practice of the present disclosure. The present disclosure is in no way limited to the methods and materials described. In the event that one or more of the incorporated references and similar materials differs from or contradicts this application, including but not limited to defined terms, term usage, described techniques, or the like, the present disclosure controls. All references, patents, patent applications cited in the present disclosure are hereby incorporated by reference in their entireties.

It is appreciated that certain features of the present disclosure, which are, for clarity, described in the context of separate embodiments, can also be provided in combination in a single embodiment. Conversely, various features of the present disclosure, which are, for brevity, described in the context of a single embodiment, can also be provided separately or in any suitable sub-combination. It must be noted that, as used in the specification and the appended claims, the singular forms “a,” “an,” and “the” include plural forms of the same unless the context clearly dictates otherwise. Thus, for example, reference to “a compound” includes a plurality of compounds.

### Definitions

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, 2^{nd} Edition, University Science Books, Sausalito, 2006; Smith and March March's Advanced Organic Chemistry, 6^{th} Edition, John Wiley & Sons, Inc., New York, 2007; Larock, Comprehensive Organic Transformations, 3^{rd} Edition, VCH Publishers, Inc., New York, 2018; Carruthers, Some Modern Methods of Organic Synthesis, 4^{th} Edition, Cambridge University Press, Cambridge, 2004; the entire contents of each of which are incorporated herein by reference.

At various places in the present disclosure, linking substituents are described. It is specifically intended that each linking substituent includes both the forward and backward forms of the linking substituent. For example, —NR(CR′R″)— includes both —NR(CR′R″)— and —(CR′R″)NR—. Where the structure clearly requires a linking group, the Markush variables listed for that group are understood to be linking groups. For example, if the structure requires a linking group and the Markush group definition for that variable lists “alkyl”, then it is understood that the “alkyl” represents a linking alkylene group.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

As used herein, a dash “—” at the front or end of a chemical group is used, a matter of convenience, to indicate a point of attachment for a substituent. For example, —OH is attached through the carbon atom; chemical groups may be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named. As used herein, a solid line coming out of the center of a ring indicates that the point of attachment for a substituent on the ring can be at any ring atom. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R¹) occurs more than one time in any constituent or formula for a compound, its definition at each occurrence is independent of its definition at every other occurrence. Thus, for example, if a group is shown to be substituted with 0-2 Rⁱ moieties, then the group may optionally be substituted with up to two Rⁱ moieties and Rⁱ at each occurrence is selected independently from the definition of Rⁱ. Also, combinations of substituents and/or variables are permissible, but only if such combinations result in stable compounds.

As used herein, the term “compounds provided herein”, or “compounds disclosed herein” or “compounds of the present disclosure” refers to the compounds of Formula (I), Formula (II), Formula (III), Formula (IV), Formula (Ia), Formula (Ib), Formula (IIa), Formula (IIb), Formula (IIIa), Formula (IIIb), Formula (IIIc), Formula (IIId), Formula (IIIe), Formula (IVa), Formula (IVb), Formula (IVc), Formula (IVd), Formula (IVe) as well as the specific compounds disclosed herein.

As used herein, the term “Cᵢ₋ⱼ” indicates a range of the carbon atoms numbers, wherein i and j are integers and the range of the carbon atoms numbers includes the endpoints (i.e. i and j) and each integer point in between, and wherein j is greater than i. For examples, C₁₋₆ indicates a range of one to six carbon atoms, including one carbon atom, two carbon atoms, three carbon atoms, four carbon atoms, five carbon atoms and six carbon atoms. In some embodiments, the term “C₁₋₁₂” indicates 1 to 12, particularly 1 to 10, particularly 1 to 8, particularly 1 to 6, particularly 1 to 5, particularly 1 to 4, particularly 1 to 3 or particularly 1 to 2 carbon atoms.

As used herein, the term “alkyl”, whether as part of another term or used independently, refers to a saturated linear or branched-chain hydrocarbon radical, which may be optionally substituted independently with one or more substituents described below. The term “Cᵢ₋ⱼ alkyl” refers to an alkyl having i to j carbon atoms. In some embodiments, alkyl groups contain 1 to 10 carbon atoms. In some embodiments, alkyl groups contain 1 to 9 carbon atoms. In some embodiments, alkyl groups contain 1 to 8 carbon atoms, 1 to 7 carbon atoms, 1 to 6 carbon atoms, 1 to 5 carbon atoms, 1 to 4 carbon atoms, 1 to 3 carbon atoms, or 1 to 2 carbon atoms. Examples of “C₁₋₁₀ alkyl” include, but are not limited to, methyl, ethyl, propyl, butyl, pentyl, hexyl, heptyl, octyl, nonyl, and decyl. Examples of “C₁₋₆ alkyl” are methyl, ethyl, propyl, isopropyl, n-butyl, i-butyl, s-butyl, t-butyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, and the like.

As used herein, the term “alkenyl”, whether as part of another term or used independently, refers to linear or branched-chain hydrocarbon radical having at least one carbon-carbon double bond, which may be optionally substituted independently with one or more substituents described herein, and includes radicals having “cis” and “trans” orientations, or alternatively, “E” and “Z” orientations. In some embodiments, alkenyl groups contain 2 to 12 carbon atoms. In some embodiments, alkenyl groups contain 2 to 11 carbon atoms. In some embodiments, alkenyl groups contain 2 to 11 carbon atoms, 2 to 10 carbon atoms, 2 to 9 carbon atoms, 2 to 8 carbon atoms, 2 to 7 carbon atoms, 2 to 6 carbon atoms, 2 to 5 carbon atoms, 2 to 4 carbon atoms, 2 to 3 carbon atoms, and in some embodiments, alkenyl groups contain 2 carbon atoms. Examples of alkenyl group include, but are not limited to, ethylenyl (or vinyl), propenyl (allyl), butenyl, pentenyl, 1-methyl-2 buten-1-yl, 5-hexenyl, and the like.

As used herein, the term “alkynyl”, whether as part of another term or used independently, refers to a linear or branched hydrocarbon radical having at least one carbon-carbon triple bond, which may be optionally substituted independently with one or more substituents described herein. In some embodiments, alkenyl groups contain 2 to 12 carbon atoms. In some embodiments, alkynyl groups contain 2 to 11 carbon atoms. In some embodiments, alkynyl groups contain 2 to 11 carbon atoms, 2 to 10 carbon atoms, 2 to 9 carbon atoms, 2 to 8 carbon atoms, 2 to 7 carbon atoms, 2 to 6 carbon atoms, 2 to 5 carbon atoms, 2 to 4 carbon atoms, 2 to 3 carbon atoms, and in some embodiments, alkynyl groups contain 2 carbon atoms. Examples of alkynyl group include, but are not limited to, ethynyl, 1-propynyl, 2-propynyl, and the like.

As used herein, the term “alkoxy”, whether as part of another term or used independently, refers to an alkyl group, as previously defined, attached to the parent molecule through an oxygen atom. The term “Cᵢ₋ⱼ alkoxy” means that the alkyl moiety of the alkoxy group has i to j carbon atoms. In some embodiments, alkoxy groups contain 1 to 10 carbon atoms. In some embodiments, alkoxy groups contain 1 to 9 carbon atoms. In some embodiments, alkoxy groups contain 1 to 8 carbon atoms, 1 to 7 carbon atoms, 1 to 6 carbon atoms, 1 to 5 carbon atoms, 1 to 4 carbon atoms, 1 to 3 carbon atoms, or 1 to 2 carbon atoms. Examples of “C₁₋₆ alkoxy” include, but are not limited to, methoxy, ethoxy, propoxy (e.g. n-propoxy and isopropoxy), t-butoxy, neopentoxy, n-hexoxy, and the like.

As used herein, the term “alkylalkoxy”, whether as part of another term or used independently, refers to an alkyl group as previously defined connected with an alkoxy group as previously defined. Alkylalkoxy group can be attached to the parent molecule through the alkyl moiety or alkoxy moiety.

As used herein, the term “amino” refers to —NH₂ group. Amino groups may also be substituted with one or more groups such as alkyl, aryl, carbonyl or other amino groups.

As used herein, the term “aryl”, whether as part of another term or used independently, refers to monocyclic and polycyclic ring systems having a total of 5 to 20 ring members, wherein at least one ring in the system is aromatic and wherein each ring in the system contains 3 to 12 ring members. Examples of “aryl” include, but are not limited to, phenyl, biphenyl, naphthyl, anthracyl and the like, which may bear one or more substituents. Also included within the scope of the term “aryl”, as it is used herein, is a group in which an aromatic ring is fused to one or more additional rings. In the case of polycyclic ring system, only one of the rings needs to be aromatic (e.g., 2,3-dihydroindole), although all of the rings may be aromatic (e.g., quinoline). The second ring can also be fused or bridged. Examples of polycyclic aryl include, but are not limited to, benzofuranyl, indanyl, phthalimidyl, naphthimidyl, phenanthridinyl, or tetrahydronaphthalenyl, and the like. Aryl groups can be substituted at one or more ring positions with substituents as described above.

As used herein, the term “cyano” refers to —CN.

As used herein, the term “cyanoalkyl” refers to an alkyl, as defined above, substituted with one or more cyano.

As used herein, the term “cycloalkyl”, whether as part of another term or used independently, refer to a monovalent non-aromatic, saturated or partially unsaturated monocyclic and polycyclic ring system, in which all the ring atoms are carbon and which contains at least three ring forming carbon atoms. In some embodiments, the cycloalkyl may contain 3 to 12 ring forming carbon atoms, 3 to 10 ring forming carbon atoms, 3 to 9 ring forming carbon atoms, 3 to 8 ring forming carbon atoms, 3 to 7 ring forming carbon atoms, 3 to 6 ring forming carbon atoms, 3 to 5 ring forming carbon atoms, 4 to 12 ring forming carbon atoms, 4 to 10 ring forming carbon atoms, 4 to 9 ring forming carbon atoms, 4 to 8 ring forming carbon atoms, 4 to 7 ring forming carbon atoms, 4 to 6 ring forming carbon atoms, 4 to 5 ring forming carbon atoms. Cycloalkyl groups may be saturated or partially unsaturated. Cycloalkyl groups may be substituted. In some embodiments, the cycloalkyl group may be a saturated cyclic alkyl group. In some embodiments, the cycloalkyl group may be a partially unsaturated cyclic alkyl group that contains at least one double bond or triple bond in its ring system. In some embodiments, the cycloalkyl group may be monocyclic or polycyclic. The fused, spiro and bridged ring systems are also included within the scope of this definition. Examples of monocyclic cycloalkyl group include, but are not limited to, cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl and cyclododecyl. Examples of polycyclic cycloalkyl group include, but are not limited to, adamantyl, norbornyl, fluorenyl, spiro-pentadienyl, spiro[3.6]-decanyl, bicyclo[1,1,1]pentenyl, bicyclo[2,2,1]heptenyl, and the like.

As used herein, the term “halogen” refers to an atom selected from fluorine (or fluoro), chlorine (or chloro), bromine (or bromo) and iodine (or iodo).

As used herein, the term “haloalkyl” refers to an alkyl, as defined above, that is substituted by one or more halogens, as defined above. Examples of haloalkyl include, but are not limited to, trifluoromethyl, difluoromethyl, trichloromethyl, 2,2,2-trifluoroethyl, 1,2-difluoroethyl, 3-bromo-2-fluoropropyl, 1,2-dibromoethyl, and the like.

As used herein, the term “heteroatom” refers to nitrogen, oxygen, or sulfur, and includes any oxidized form of nitrogen or sulfur, and any quaternized form of a basic nitrogen (including N-oxides).

As used herein, the term “heteroalkyl” refers to an alkyl, at least one of the carbon atoms of which is replaced with a heteroatom selected from N, O, or S. The heteroalkyl may be a carbon radical or heteroatom radical (i.e., the heteroatom may appear in the middle or at the end of the radical), and may be optionally substituted independently with one or more substituents described herein. The term “heteroalkyl” encompasses alkoxy and heteroalkoxy radicals.

As used herein, the term “heteroalkenyl” refers to an alkenyl, at least one of the carbon atoms of which is replaced with a heteroatom selected from N, O, or S. The heteroalkenyl may be a carbon radical or heteroatom radical (i.e., the heteroatom may appear in the middle or at the end of the radical), and may be optionally substituted independently with one or more substituents described herein.

As used herein, the term “heteroalkynyl” refers to an alkynyl, at least one of the carbon atoms of which is replaced with a heteroatom selected from N, O, or S. The heteroalkynyl may be a carbon radical or heteroatom radical (i.e., the heteroatom may appear in the middle or at the end of the radical), and may be optionally substituted independently with one or more substituents described herein.

As used herein, the term “heteroaryl”, whether as part of another term or used independently, refers to an aryl group having, in addition to carbon atoms, one or more heteroatoms. The heteroaryl group can be monocyclic. Examples of monocyclic heteroaryl include, but are not limited to, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, benzofuranyl and pteridinyl. The heteroaryl group also includes polycyclic groups in which a heteroaromatic ring is fused to one or more aryl, cycloaliphatic, or heterocyclyl rings, where the radical or point of attachment is on the heteroaromatic ring. Examples of polycyclic heteroaryl include, but are not limited to, indolyl, isoindolyl, benzothienyl, benzofuranyl, benzo[1,3]dioxolyl, dibenzofuranyl, indazolyl, benzimidazolyl, benzothiophenyl, benzthiazolyl, quinolyl, isoquinolyl, dihydroquinolinyl, dihydroisoquinolinyl, tetrahydroquinolinyl, tetrahydroisoquinolinyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and the like.

As used herein, the term “heterocyclyl” refers to a saturated or partially unsaturated carbocyclyl group in which one or more ring atoms are heteroatoms independently selected from oxygen, sulfur, nitrogen, phosphorus, and the like, the remaining ring atoms being carbon, wherein one or more ring atoms may be optionally substituted independently with one or more substituents. In some embodiments, the heterocyclyl is a saturated heterocyclyl. In some embodiments, the heterocyclyl is a partially unsaturated heterocyclyl having one or more double bonds in its ring system. In some embodiments, the heterocyclyl may contains any oxidized form of carbon, nitrogen or sulfur, and any quaternized form of a basic nitrogen. “Heterocyclyl” also includes radicals wherein the heterocyclyl radicals are fused with a saturated, partially unsaturated, or fully unsaturated (i.e., aromatic) carbocyclic or heterocyclic ring. The heterocyclyl radical may be carbon linked or nitrogen linked where such is possible. In some embodiments, the heterocycle is carbon linked. In some embodiments, the heterocycle is nitrogen linked. For example, a group derived from pyrrole may be pyrrol-1-yl (nitrogen linked) or pyrrol-3-yl (carbon linked). Further, a group derived from imidazole may be imidazol-1-yl (nitrogen linked) or imidazol-3-yl (carbon linked).

In some embodiments, the term “3- to 12-membered heterocyclyl” refers to a 3- to 12-membered saturated or partially unsaturated monocyclic or polycyclic heterocyclic ring system having 1 to 3 heteroatoms independently selected from nitrogen, oxygen, or sulfur. The fused, spiro and bridged ring systems are also included within the scope of this definition. Examples of monocyclic heterocyclyl include, but are not limited to oxetanyl, 1,1-dioxothietanylpyrrolidyl, tetrahydrofuryl, tetrahydrothienyl, pyrrolyl, furanyl, thienyl, pyrazolyl, imidazolyl, triazolyl, oxazolyl, thiazolyl, piperidyl, piperazinyl, piperidinyl, morpholinyl, pyridinyl, pyrazinyl, pyrimidinyl, pyridazinyl, triazinyl, pyridonyl, pyrimidonyl, pyrazinonyl, pyrimidonyl, pyridazonyl, pyrrolidinyl, triazinonyl, and the like. Examples of fused heterocyclyl include, but are not limited to, phenyl fused ring or pyridinyl fused ring, such as quinolinyl, isoquinolinyl, tetrahydroquinolinyl, tetrahydroisoquinolinyl, quinoxalinyl, quinolizinyl, quinazolinyl, azaindolizinyl, pteridinyl, chromenyl, isochromenyl, indolyl, isoindolyl, indolizinyl, indazolyl, purinyl, benzofuranyl, isobenzofuranyl, benzimidazolyl, benzothienyl, benzothiazolyl, carbazolyl, phenazinyl, phenothiazinyl, phenanthridinyl, hexahydro-1H-pyrrolizinyl, imidazo[1,2-a]pyridinyl, [1,2,4]triazolo[4,3-a]pyridinyl, [1,2,3]triazolo[4,3-a]pyridinyl groups, and the like. Examples of spiro heterocyclyl include, but are not limited to, spiropyranyl, spirooxazinyl, and the like. Examples of bridged heterocyclyl include, but are not limited to, morphanyl, hexamethylenetetraminyl, 3-aza-bicyclo[3.1.0]hexane, 8-aza-bicyclo[3.2.1]octane, 1-aza-bicyclo[2.2.2]octane, 1,4-diazabicyclo[2.2.2]octane (DABCO), and the like.

As used herein, the term “alkylcycloalkylalkyl” refers to alkyl-cycloalkyl-alkyl, which is attached to the parent molecule through any of the alkyl groups, wherein the alkyl and cycloalkyl are as defined above.

As used herein, the term “alkylheterocyclylalkyl” refers to alkyl-heterocyclyl-alkyl, which is attached to the parent molecule through any of the alkyl groups, wherein the alkyl and heterocyclyl are as defined above.

As used herein, the term “hydroxyl” or “hydroxy” refers to —OH.

As used herein, the term “hydroxyalkyl” refers to an alkyl, as defined above, substituted with one or more hydroxyl.

As used herein, the term “oxo” refers to ═O substituent.

As used herein, the term “partially unsaturated” refers to a radical that includes at least one double or triple bond. The term “partially unsaturated” is intended to encompass rings having multiple sites of unsaturation, but is not intended to include aromatic (i.e., fully unsaturated) moieties.

As used herein, the term “substituted”, whether preceded by the term “optionally” or not, means that one or more hydrogens of the designated moiety are replaced with a suitable substituent. It will be understood that “substitution” or “substituted with” includes the implicit proviso that such substitution is in accordance with permitted valence of the substituted atom and that the substitution results in a stable or chemically feasible compound, e.g., which does not spontaneously undergo transformation such as by rearrangement, cyclization, elimination, etc. Unless otherwise indicated, an “optionally substituted” group may have a suitable substituent at each substitutable position of the group, and when more than one position in any given structure may be substituted with more than one substituent selected from a specified group, the substituent may be either the same or different at every position. It will be understood by those skilled in the art that substituents can themselves be substituted, if appropriate. Unless specifically stated as “unsubstituted”, references to chemical moieties herein are understood to include substituted variants. For example, reference to an “aryl” group or moiety implicitly includes both substituted and unsubstituted variants.

### Compounds

In one aspect, the present disclosure provides a compound having Formula (I) or Formula (I′):

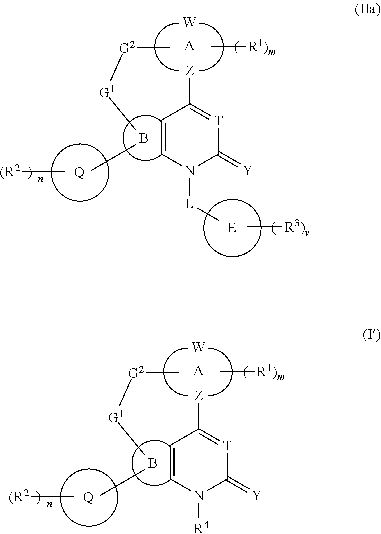

or a pharmaceutically acceptable salt thereof,
wherein
   Y is O or S;
   T is N or C(R^{T});
   R^{T} is selected from hydrogen, halogen, hydroxyl, cyano, alkyl, heteroalkyl, haloalkyl or —OR^{b};
   Ring A is heterocyclyl or heteroaryl,
   Z is C(R^{e}) or N;
   R^{e} is absent or hydrogen;
   W is N(R^{1W}) or C(R^{2W})(R^{3W});
   R^{1W} is absent, hydrogen, hydroxyl, alkyl, cycloalkyl, heterocyclyl or heteroalkyl;
   R^{2W} is absent, hydrogen, alkyl or cycloalkyl;
   R^{3W} is hydrogen, hydroxyl, —N(R^{a})₂, alkyl or heteroalkyl;
   each R¹ is independently selected from the group consisting of hydrogen, oxo, hydroxyl, halogen, cyano, alkyl, alkenyl, alkynyl, alkylalkoxy, heteroalkyl, heteroaryl, —C(O)R*, —C(O)OR*, —C(O)N(R^{a})₂ and —N(R^{a})₂, wherein the alkyl, alkenyl, alkynyl, alkylalkoxy, heteroalkyl and heteroaryl are optionally substituted with one or more groups independently selected from deuterium, cyano, hydroxyl, halogen, —OR^{b}, or —N(R^{b})₂, provided that if R¹ is present, R¹ is not connected to W;
   each R^{a} and R^{b} is independently hydrogen, alkyl, alkenyl or alkynyl, wherein the alkyl and alkenyl are optionally substituted with one or more deuterium;
   R* is selected from hydrogen, alkyl, alkylaryl or aryl;
   Ring B is selected from cycloalkyl, heterocyclyl, aryl, or heteroaryl, each of which is optionally substituted with one or more R′;
   each R′ is independently selected from the group consisting of oxo, hydroxyl, halogen, cyano, amino, alkyl, alkenyl, alkynyl, heteroalkyl, heteroalkenyl, heteroalkynyl, heteroaryl, wherein alkyl, alkenyl, alkynyl, heteroalkyl, heteroalkenyl, heteroalkynyl and heteroaryl are optionally substituted with one or more group independently consisting of deuterium, hydroxyl, halogen, cyano, —OR^{a}, —N(R^{a})₂, and heteroaryl;
   Ring Q is selected from cycloalkyl, heterocyclyl, aryl or heteroaryl;
   each R² is independently selected from the group consisting of hydrogen, oxo, hydroxyl, halogen, cyano, amino, nitro, alkyl, alkenyl, alkynyl, alkoxy, haloalkyl, heteroalkyl, heteroalkenyl, heteroalkynyl, cycloalkyl, heterocyclyl, aryl, heteroaryl and —C(O)R*, wherein alkyl, alkenyl, alkynyl, alkoxy, heteroalkyl, heteroalkenyl, heteroalkynyl, cycloalkyl, heterocyclyl, aryl, and heteroaryl are optionally substituted with one or more group independently consisting of deuterium, hydroxyl, halogen, cyano, amino, nitro, alkyl, alkoxy, haloalkyl, hydroxyalkyl, cycloalkyl, heterocyclyl, aryl and heteroaryl;
   G¹ is a bond, —[C(R^{d})₂]ᵤ—, —O—, —S(O)ₚ—, —S—S—, —N(R^{c})—, or —C(R^{d})═C(R^{d})—;
   G² is a bond, —[C(R^{d})₂]ᵤ—, —N(R^{c})—, —C(O)— or —C(O)C(R^{d})₂—;
   R^{c} is selected from the group consisting of hydrogen, alkyl, haloalkyl, hydroxyalkyl, heteroalkyl, cycloalkyl and heterocyclyl, wherein the alkyl, haloalkyl, hydroxyalkyl, heteroalkyl, cycloalkyl and heterocyclyl are optionally substituted with one or more deuterium;
   each R^{d} is independently selected from the group consisting of hydrogen, hydroxyl, halogen, cyano, amino, nitro, alkyl, alkenyl, alkynyl, alkoxy, heteroalkyl, heteroalkenyl, heteroalkynyl, cycloalkyl, and heterocyclyl, wherein alkyl, alkenyl, alkynyl, alkoxy, heteroalkyl, heteroalkenyl, heteroalkynyl, cycloalkyl, and heterocyclyl are optionally substituted with one or more group independently consisting of deuterium, hydroxyl, halogen, cyano, amino, nitro, alkyl, alkoxy, haloalkyl, and hydroxyalkyl; or
   two R^{d} together with the carbon atom to which they are both attached form a cycloalkyl or heterocyclyl, wherein cycloalkyl and heterocyclyl are optionally substituted with cyano, halogen, hydroxyl, amino, nitro, alkoxy, haloalkyl, hydroxyalkyl and alkyl;
   L is selected from alkyl, alkenyl, alkynyl, alkylcycloalkylalkyl, or alkylheterocyclylalkyl, each of which is optionally substituted with one or more R^{f};
   each R^{f} is independently selected from deuterium, alkyl, hydroxyl, halogen, cyano or amino, wherein the alkyl and amino are optionally substituted with one or more groups independently selected from deuterium, hydroxyl and alkyl;
   Ring E is selected from cycloalkyl, heterocyclyl, aryl or heteroaryl;
   each R³ is independently selected from the group consisting of hydrogen, deuterium, oxo, hydroxyl, halogen, alkyl, alkoxy, haloalkyl, hydroxyalkyl, cycloalkyl, heterocyclyl, —N(R^{a})₂ and ═C(R″)₂, wherein the alkyl, alkoxy, haloalkyl, hydroxyalkyl, cycloalkyl, heterocyclyl are optionally substituted with one or more groups selected from deuterium, cycloalkyl or heterocyclyl; each R^{g} is independently hydrogen, alkyl, alkenyl or alkynyl;
   each R″ is independently selected from hydrogen, hydroxyl, halogen, cyano, alkyl, cycloalkyl, hydroxyalkyl, haloalkyl or heterocyclyl;
   R⁴ is alkyl optionally substituted with one or more group independently selected from deuterium, halogen, hydroxyl, hydroxyalkyl, alkyl, alkoxy, —N(R^{h})₂ or -alkyl-N(R^{h})₂;
   each R^{h} is independently hydrogen, alkyl, alkenyl or alkynyl;
   m is an integer from 0 to 6;
   n is an integer from 0 to 5;
   p is an integer from 0 to 2;
   u is an integer from 0 to 4; and
   v is an integer from 0 to 4.

In some embodiments, Z is C(R^{e}). In certain embodiments, R^{e} is absent. In certain embodiments, R^{e} is hydrogen.

In some embodiments, Z is N.

In some embodiments, W is N(R^{1W}). In certain embodiments, R^{1W} is hydrogen. In certain embodiments, R^{1W} is hydroxyl. In certain embodiments, R^{1W} is alkyl. In certain embodiments, R^{1W} is heteroalkyl.

In some embodiments, W is C(R^{2W})(R^{3W}). In certain embodiments, R^{2W} is hydrogen, R^{3W} is hydrogen, —N(R^{a})₂, hydroxyl or alkyl. In certain embodiments, R^{2W} is hydrogen, R^{3W} is hydrogen, —NH₂, hydroxyl or C₁₋₆ alkyl. In certain embodiments, R^{2W} is C₁₋₃ alkyl, R^{3W} is hydrogen, —N(R^{a})₂, hydroxyl or alkyl. In certain embodiments, R^{2W} is C₁₋₃ alkyl, R^{3W} is hydrogen, —NH₂, hydroxyl or C₁₋₃ alkyl.

In some embodiments, Ring A is heteroaryl. In certain embodiments, Ring A is a 6- to 12-membered heteroaryl. In certain embodiments, Ring A is a 6- to 10-membered heteroaryl. In certain embodiments, Ring A is a 8- to 10-membered heteroaryl.

In some embodiments, Ring A is heterocyclyl. In certain embodiments, Ring A is a 5- to 13-membered heterocyclyl. In certain embodiments, Ring A is a 6- to 12-membered heterocyclyl. In certain embodiments, Ring A is a 7- to 11-membered heterocyclyl. In certain embodiments, Ring A is a 8- to 10-membered heterocyclyl.

In some embodiments, Ring A is a bridged heterocyclyl optionally containing at least one further heteroatom selected from the group consisting of N, S and O. In certain embodiments, Ring A is a 5- to 13-membered bridged heterocyclyl optionally containing at least one further heteroatom selected from the group consisting of N, S and O. In certain embodiments, Ring A is a 6- to 12-membered bridged heterocyclyl optionally containing at least one further heteroatom selected from the group consisting of N, S and O. In certain embodiments, Ring A is a 7- to 11-membered bridged heterocyclyl optionally containing at least one further heteroatom selected from the group consisting of N, S and O. In certain embodiments, Ring A is a 8- to 10-membered bridged heterocyclyl optionally containing at least one further heteroatom selected from the group consisting of N, S and O.

In some embodiments, Ring A is a spiro or fused ring optionally containing at least one further heteroatom selected from the group consisting of N, S and O. In certain embodiments, Ring A is a 5- to 13-membered spiro or fused heterocyclyl optionally containing at least one further heteroatom selected from the group consisting of N, S and O. In certain embodiments, Ring A is a 6- to 12-membered spiro or fused heterocyclyl optionally containing at least one further heteroatom selected from the group consisting of N, S and O. In certain embodiments, Ring A is a 7- to 11-membered spiro or fused heterocyclyl optionally containing at least one further heteroatom selected from the group consisting of N, S and O. In certain embodiments, Ring A is a 8- to 10-membered spiro or fused heterocyclyl optionally containing at least one further heteroatom selected from the group consisting of N, S and O.

In certain embodiments, Ring A is selected from the group consisting of:

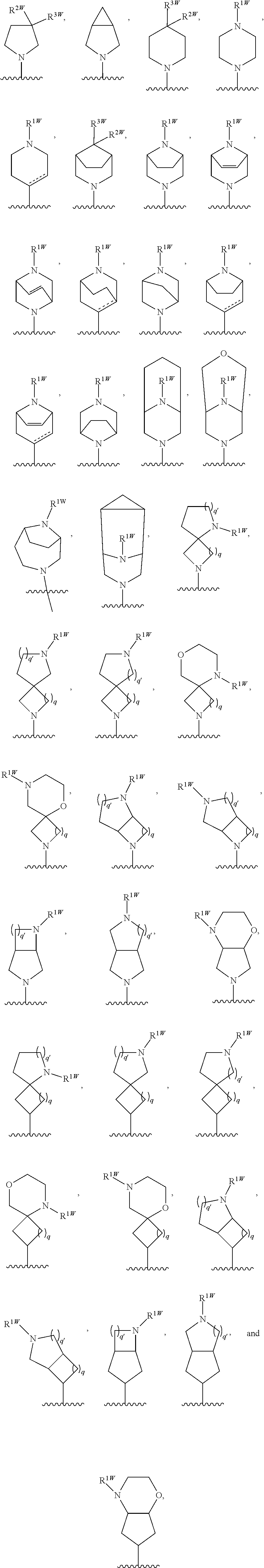

wherein [symbol] represents a single bond or a double bond, q is an integer from 1 to 4, and q′ is an integer from 0 to 4.

In certain embodiments, Ring A is selected from the group consisting of:

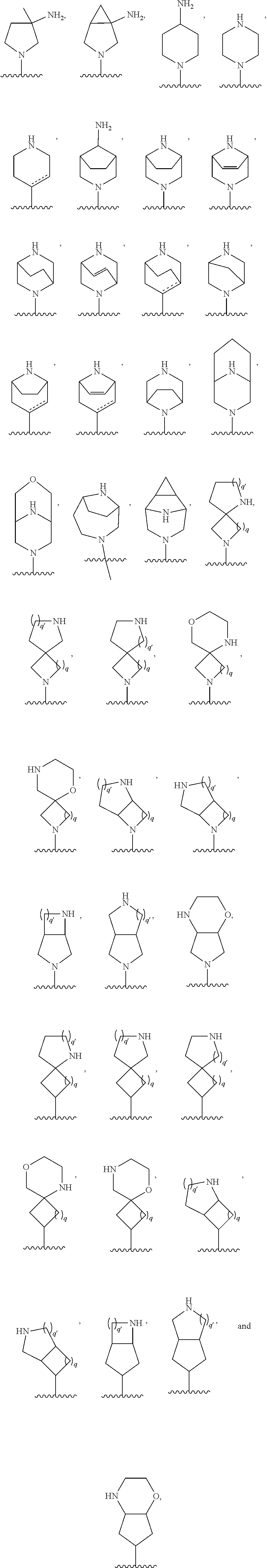

wherein [symbol] represents a single bond or a double bond, q is an integer from 1 to 4, and q′ is an integer from 0 to 4.

In some embodiments, Ring B is cycloalkyl optionally substituted with one or more R′. In certain embodiments, Ring B is C₅₋₁₂ cycloalkyl optionally substituted with one or more R′. In certain embodiments, Ring B is C₅₋₁₀ cycloalkyl optionally substituted with one or more R′. In certain embodiments, Ring B is C₅₋₈ cycloalkyl optionally substituted with one or more R′. In certain embodiments, Ring B is C₅₋₇ cycloalkyl optionally substituted with one or more R′. In certain embodiments, Ring B is C₅₋₆ cycloalkyl optionally substituted with one or more R′. In certain embodiments, each R′ is independently halogen.

In some embodiments, Ring B is heterocyclyl optionally substituted with one or more R′. In certain embodiments, Ring B is 5- to 12-membered heterocyclyl optionally substituted with one or more R′. In certain embodiments, Ring B is 5- to 10-membered heterocyclyl optionally substituted with one or more R′. In certain embodiments, Ring B is 5- to 8-membered heterocyclyl optionally substituted with one or more R′. In certain embodiments, Ring B is 5- to 7-membered heterocyclyl optionally substituted with one or more R′. In certain embodiments, Ring B is 5- to 6-membered heterocyclyl optionally substituted with one or more R′. In certain embodiments, each R′ is independently halogen.

In certain embodiments, Ring B is 1,2,3,6-tetrahydropyridinyl or piperidinyl, each optionally substituted with one or more R′ independently selected from oxo, alkyl, alkynyl, heteroalkyl, or cyano. In certain embodiments, each R′ is independently halogen.

In some embodiments, Ring B is aryl optionally substituted with one or more R′. In certain embodiments, Ring B is C₅₋₁₂ aryl optionally substituted with one or more R′. In certain embodiments, Ring B is C₅₋₁₀ aryl optionally substituted with one or more R′. In certain embodiments, Ring B is C₅₋₈ aryl optionally substituted with one or more R′. In certain embodiments, Ring B is C₅₋₇ aryl optionally substituted with one or more R′. In certain embodiments, Ring B is C₅₋₆ aryl optionally substituted with one or more R′. In certain embodiments, each R′ is independently halogen.

In certain embodiments, Ring B is phenyl optionally substituted with one or more R′. In certain embodiments, each R′ is independently halogen.

In some embodiments, Ring B is heteroaryl optionally substituted with one or more R′. In certain embodiments, Ring B is 5- to 12-membered heteroaryl optionally substituted with one or more R′. In certain embodiments, Ring B is 5- to 10-membered heteroaryl optionally substituted with one or more R′. In certain embodiments, Ring B is 5- to 8-membered heteroaryl optionally substituted with one or more R′. In certain embodiments, Ring B is 5- to 7-membered heteroaryl optionally substituted with one or more R′. In certain embodiments, Ring B is 5- to 6-membered heteroaryl optionally substituted with one or more R′. In certain embodiments, each R′ is independently halogen.

In certain embodiments, Ring B is pyridinyl or pyrimidinyl, each optionally substituted with one or more R′. In certain embodiments, each R′ is independently halogen.

In some embodiments, Ring Q is cycloalkyl. In certain embodiments, Ring Q is C₅₋₁₂ cycloalkyl. In certain embodiments, Ring Q is C₅₋₁₀ cycloalkyl. In certain embodiments, Ring Q is C₅₋₈ cycloalkyl. In certain embodiments, Ring Q is C₅₋₇ cycloalkyl. In certain embodiments, Ring Q is C₅₋₆ cycloalkyl.

In some embodiments, Ring Q is heterocyclyl. In certain embodiments, Ring Q is 5- to 12-membered heterocyclyl. In certain embodiments, Ring Q is 5- to 10-membered heterocyclyl. In certain embodiments, Ring Q is 5- to 8-membered heterocyclyl. In certain embodiments, Ring Q is 5- to 7-membered heterocyclyl. In certain embodiments, Ring Q is 5- to 6-membered heterocyclyl.

In some embodiments, Ring Q is aryl. In certain embodiments, Ring Q is C₅₋₁₂ aryl. In certain embodiments, Ring Q is C₅₋₁₀ aryl. In certain embodiments, Ring Q is C₅₋₈ aryl. In certain embodiments, Ring Q is C₅₋₇ aryl. In certain embodiments, Ring Q is C₅₋₆ aryl.

In certain embodiments, Ring Q is phenyl, naphthalenyl or tetrahydronaphthalenyl.

In some embodiments, Ring Q is heteroaryl. In certain embodiments, Ring Q is 5- to 12-membered heteroaryl. In certain embodiments, Ring Q is 5- to 10-membered heteroaryl. In certain embodiments, Ring Q is 5- to 8-membered heteroaryl. In certain embodiments, Ring Q is 5- to 7-membered heteroaryl. In certain embodiments, Ring Q is 5- to 6-membered heteroaryl.

In certain embodiments, Ring Q is selected from benzothiophenyl, benzoimidazolyl, quinazolinyl, benzotriazolyl, thiophenyl, thienopyridinyl, isoquinolinyl, indolyl, or indazolyl.

In some embodiments, G¹ is a bond.

In some embodiments, G¹ is —[C(R^{d})₂]ᵤ—. In certain embodiments, each R^{d} is independently selected from hydrogen, halogen, hydroxyl, amino or alkyl optionally substituted with one or more deuterium. In certain embodiments, each R^{d} is independently hydrogen or alkyl optionally substituted with one or more deuterium. In certain embodiments, u is 1 or 2. In certain embodiments, G¹ is —CH₂—.

In some embodiments, G¹ is —O—.

In some embodiments, G¹ is —S(O)ₚ— or —S—S—.

In some embodiments, G¹ is —N(R^{c})—. In certain embodiments, R^{c} is hydrogen. In certain embodiments, R^{c} is alkyl. In certain embodiments, R^{c} is C₁₋₆ alkyl, C₁₋₅ alkyl, C₁₋₄ alkyl, C₁₋₃ alkyl. In certain embodiments, R^{c} is methyl.

In some embodiments, G¹ is —C(R^{d})═C(R^{d})—. In certain embodiments, each R^{d} is independently hydrogen or alkyl. In certain embodiments, both R^{d} are hydrogen. In certain embodiments, both R^{d} are alkyl, such as C₁₋₆ alkyl, C₁₋₅ alkyl, C₁₋₄ alkyl, or C₁₋₃ alkyl. In certain embodiments, one R^{d} is hydrogen and the other is alkyl. In certain embodiments, one R^{d} is hydrogen and the other is methyl.

In some embodiments, G² is a bond.

In some embodiments, G² is —[C(R^{d})₂]ᵤ—. In certain embodiments, each R^{d} is independently hydrogen, hydroxyl or alkyl optionally substituted with one or more deuterium. In certain embodiments, each R^{d} is independently hydrogen, hydroxyl, C₁₋₆ alkyl, C₁₋₅ alkyl, C₁₋₄ alkyl, or C₁₋₃ alkyl, wherein the alkyl is optionally substituted with one or more deuterium. In certain embodiments, each R^{d} is independently hydrogen, hydroxyl or methyl optionally substituted with one or more deuterium. In certain embodiments, each R^{d} is independently hydrogen, hydroxyl, —CH₃ or -CD₃.

In some embodiments, G² is —C(O)—.

In some embodiments, G² is —C(O)C(R^{d})₂—. In certain embodiments, each R^{d} is independently hydrogen or alkyl optionally substituted with one or more deuterium. In certain embodiments, both R^{d} are hydrogen. In certain embodiments, both R^{d} are alkyl (such as C₁₋₆ alkyl, C₁₋₅ alkyl, C₁₋₄ alkyl, or C₁₋₃ alkyl) optionally substituted with one or more deuterium. In certain embodiments, one R^{d} is hydrogen and the other is alkyl optionally substituted with one or more deuterium. In certain embodiments, one R^{d} is hydrogen and the other is —CH₃ or -CD₃.

In some embodiments, G¹ is —[C(R^{d})₂]ᵤ— or —O—, and G² is —[C(R^{d})₂]ᵤ— or —N(R^{c})—. In certain embodiments, u is 1 or 2. In certain embodiments, each of R^{c} and R^{d} is independently hydrogen or alkyl optionally substituted with one or more deuterium. In certain embodiments, each of R^{c} and R^{d} is independently hydrogen, C₁₋₆ alkyl, C₁₋₅ alkyl, C₁₋₄ alkyl, or C₁₋₃ alkyl, wherein the alkyl is optionally substituted with one or more deuterium. In certain embodiments, each of R^{c} and R^{d} is independently hydrogen, —CH₃ or -CD₃.

In some embodiments, G¹ is —[C(R^{d})₂]ᵤ— and G² is —N(R^{c})—. In certain embodiments, u is 1 or 2. In certain embodiments, each of R^{c} and R^{d} is independently hydrogen or alkyl optionally substituted with one or more deuterium. In certain embodiments, each of R^{c} and R^{d} is independently hydrogen, C₁₋₆ alkyl, C₁₋₅ alkyl, C₁₋₄ alkyl, or C₁₋₃ alkyl, wherein the alkyl is optionally substituted with one or more deuterium. In certain embodiments, each of R^{c} and R^{d} is independently hydrogen, —CH₃ or -CD₃.

In some embodiments, -G¹-G²- is —CH₂—N(CH₃)—.

In some embodiments, G¹ is —O—, and G² is —[C(R^{d})₂]ᵤ—. In certain embodiments, u is 1 or 2. In certain embodiments, each R^{d} is independently hydrogen or alkyl optionally substituted with one or more deuterium. In certain embodiments, each R^{d} is independently hydrogen, C₁₋₆ alkyl, C₁₋₅ alkyl, C₁₋₄ alkyl, or C₁₋₃ alkyl, wherein the alkyl is optionally substituted with one or more deuterium. In certain embodiments, each R^{d} is independently hydrogen, —CH₃ or -CD₃.

In some embodiments, -G¹-G²- is —O—CH₂—, —O—CH(CH₃)— or —O—CH(CD₃)—.

In some embodiments, G¹ is —O—, and G² is —[C(R^{d})₂]ᵤ— wherein u is 1 or 2. In certain embodiments, each R^{d} is independently hydrogen or alkyl optionally substituted with one or more deuterium. In certain embodiments, each R^{d} is independently hydrogen, C₁₋₆ alkyl, C₁₋₅ alkyl, C₁₋₄ alkyl, or C₁₋₃ alkyl, wherein the alkyl is optionally substituted with one or more deuterium. In certain embodiments, each R^{d} is independently hydrogen, —CH₃ or -CD₃.

In some embodiments, G¹ is —S(O)ₚ—, and G² is —[C(R^{d})₂]ᵤ— wherein u is 1. In certain embodiments, each R^{d} is independently hydrogen or alkyl. In certain embodiments, each R^{d} is independently hydrogen, C₁₋₆ alkyl, C₁₋₅ alkyl, C₁₋₄ alkyl, or C₁₋₃ alkyl. In certain embodiments, each R^{d} is independently hydrogen or methyl. In certain embodiments, both R^{d} are hydrogen.

In some embodiments, G¹ is —N(R^{c})—, and G² is —C(O)—, —C(O)C(R^{d})₂— or —[C(R^{d})₂]ᵤ— wherein u is 1 or 2. In certain embodiments, each R^{d} is independently hydrogen or alkyl optionally substituted with one or more deuterium. In certain embodiments, each R^{d} is independently hydrogen, C₁₋₆ alkyl, C₁₋₅ alkyl, C₁₋₄ alkyl, or C₁₋₃ alkyl, wherein the alkyl is optionally substituted with one or more deuterium. In certain embodiments, each R^{d} is independently hydrogen, —CH₃ or -CD₃.

In some embodiments, G¹ is —C(R^{d})═C(R^{d})—, G² is —C(O)— or —[C(R^{d})₂]ᵤ— wherein u is 1. In certain embodiments, each R^{d} is independently hydrogen, hydroxyl, C₁₋₆ alkyl, C₁₋₅ alkyl, C₁₋₄ alkyl, or C₁₋₃ alkyl, wherein the alkyl is optionally substituted with one or more deuterium. In certain embodiments, each R^{d} is independently hydrogen, hydroxyl, —CH₃ or -CD₃.

In some embodiments, m is 0.

In some embodiments, m is an integer from 1 to 3, and each R¹ is independently hydrogen, halogen, alkyl (such as C₁₋₆ alkyl, C₁₋₅ alkyl, C₁₋₄ alkyl, or C₁₋₃ alkyl), alkenyl (such as C₂₋₆ alkenyl, C₂₋₅ alkenyl, C₂₋₄ alkenyl, or C₂₋₃ alkenyl), or alkylalkoxy (such as C₂₋₁₀ alkylalkoxy, C₂₋₉ alkylalkoxy, C₂₋₈ alkylalkoxy, C₂₋₇ alkylalkoxy, C₂₋₆ alkylalkoxy, C₂₋₅ alkylalkoxy, C₂₋₄ alkylalkoxy, or C₂₋₃ alkylalkoxy), wherein the alkyl, alkenyl and alkylalkoxy are optionally substituted with one or more deuterium.

In some embodiments, m is 1, and R¹ is alkyl, alkenyl, alkylalkoxy, —C(O)R* or —C(O)OR*, wherein the alkyl, alkenyl and alkylalkoxy are optionally substituted with one or more deuterium, and R* is alkyl or alkylaryl. In certain embodiments, m is 1, and R¹ is —CH₃, —CD₃, —CH═CH₂, —CH₂—O—CH₃, —CH₂—O-CD₃, —C(O)R* or —C(O)OR*, wherein R* is alkyl or alkylaryl.

In some embodiments,

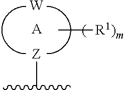

is selected from the group consisting of:

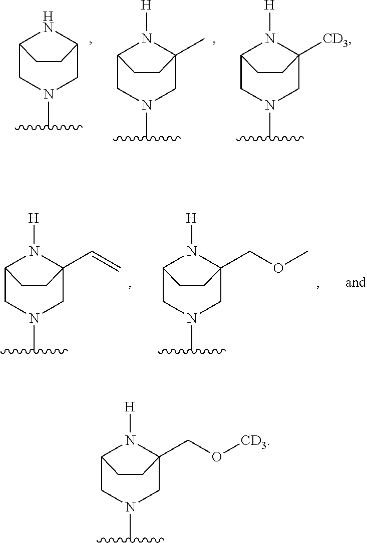

In some embodiments, n is an integer from 1 to 4, and each R² is independently selected from hydroxyl, halogen, cyano, amino, alkyl, alkenyl, alkynyl, or cycloalkyl, wherein alkyl, alkenyl, alkynyl, and cycloalkyl are optionally substituted with one or more groups independently selected from cyano, hydroxyl, halogen, or alkyl.

In certain embodiments, n is an integer from 1 to 4, and each R² is independently selected from hydroxyl, halogen, amino, C₁₋₃ alkyl, C₂₋₄ alkynyl, C₁₋₃ haloalkyl, or C₃₋₆ cycloalkyl.

In some embodiments, L is

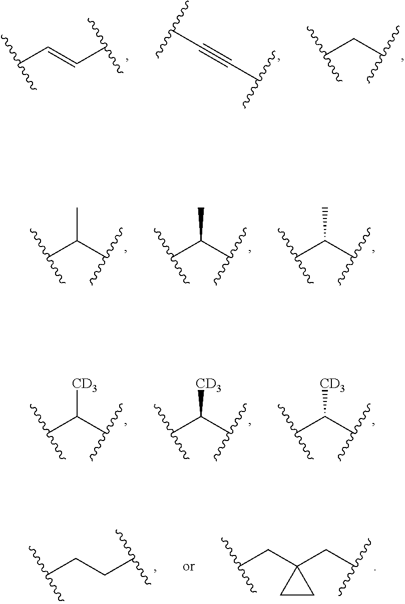

In some embodiments, Ring E is heterocyclyl. In certain embodiments, Ring E is 5- to 12-membered heterocyclyl, 5- to 11-membered heterocyclyl, 5- to 10-membered heterocyclyl, 5- to 9-membered heterocyclyl, 5- to 8-membered heterocyclyl, 5- to 7-membered heterocyclyl, or 5- to 6-membered heterocyclyl.

In some embodiments, Ring E is selected from the group consisting of:

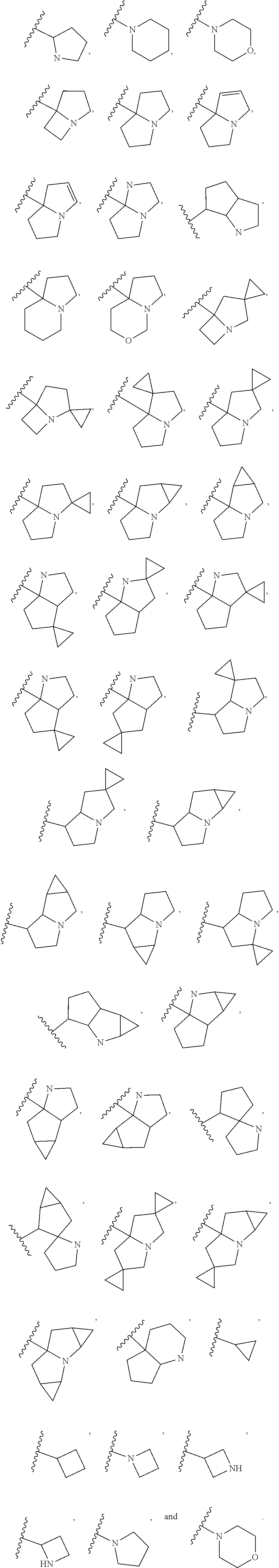

In some embodiments, R³ is selected from hydrogen, halogen, alkyl, alkoxy, hydroxyalkyl, heterocyclyl, —N(R^{g})₂ or ═C(R″)₂, wherein the alkyl, alkoxy, hydroxyalkyl, and heterocyclyl are optionally substituted with one or more groups selected from deuterium. In certain embodiments, each R″ is independently selected from hydrogen, hydroxyl, halogen, or alkyl (such as C₁₋₅ alkyl, C₁₋₄ alkyl, C₁₋₃ alkyl, or C₁₋₂ alkyl). In certain embodiment, each R⁹ is independently hydrogen or alkyl (such as C₁₋₅ alkyl, C₁₋₄ alkyl, C₁₋₃ alkyl, or C₁₋₂ alkyl).

In some embodiments, v is an integer from 1 to 4, at least one R³ is ═C(R″)₂. In certain embodiments, each R″ is independently selected from hydrogen, hydroxyl, halogen, or alkyl.

In some embodiments, v is an integer from 1 to 4, at least one R³ is ═CH₂, ═CHF, or ═CF₂.

In some embodiments, v is an integer from 2 to 4, one R³ is ═C(R″)₂, and other R³ is independently selected from hydroxyl, halogen, alkyl, alkoxy, or ═C(R″)₂.

In some embodiments, v is an integer from 1 to 4, each R³ is independently selected from F, —CH₃, —CD₃, ═CH₂, ═CHF, ═CF₂, —CH₂OH, —OCH₃, —N(CH₃)₂,

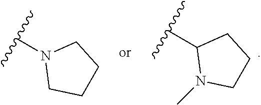

In some embodiments,

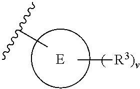

is selected from the group consisting of:

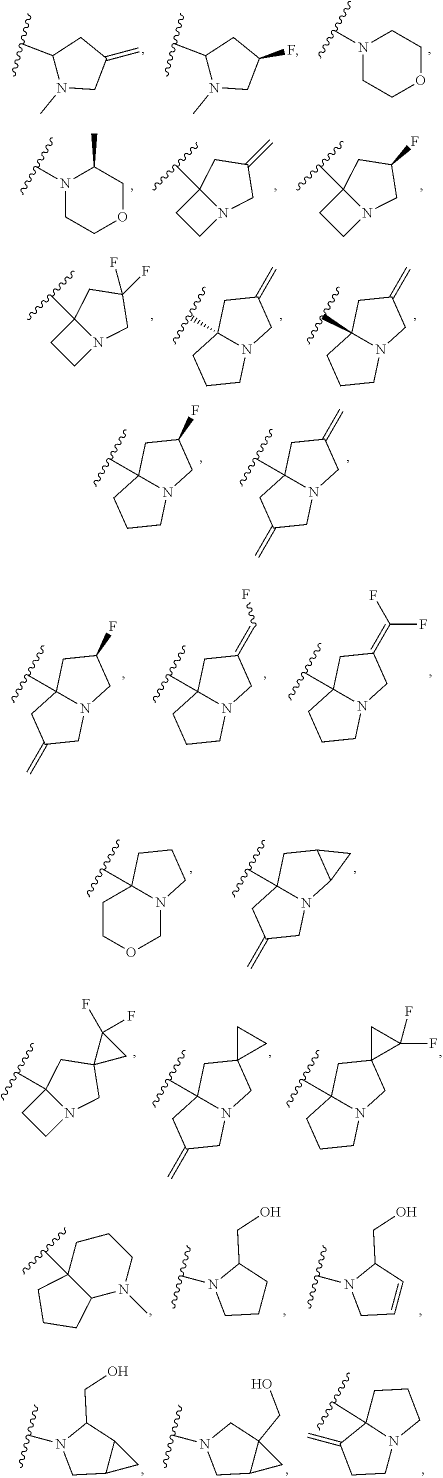

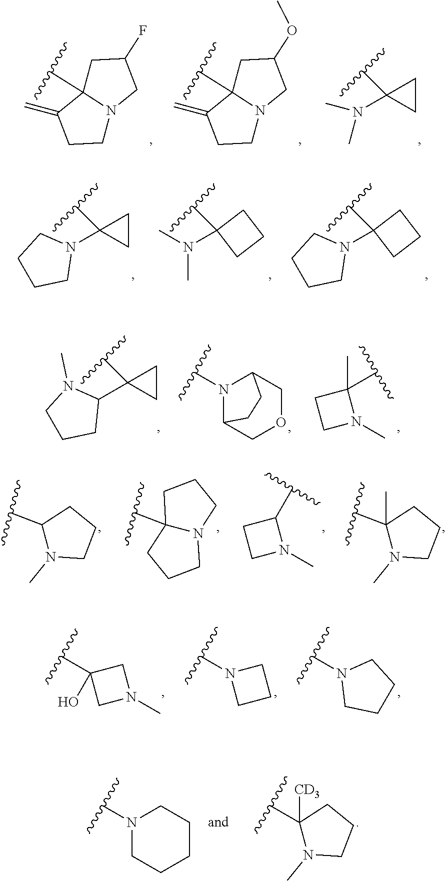

In some embodiments, Ring E is cycloalkyl. In certain embodiments, Ring E is C₃₋₁₂ cycloalkyl. In certain embodiments, Ring E is C₃₋₁₀ cycloalkyl. In certain embodiments, Ring E is C₃_cycloalkyl. In certain embodiments, Ring E is C₃₋₇ cycloalkyl. In certain embodiments, Ring E is C₃₋₆ cycloalkyl. In certain embodiments, Ring E is C₃₋₅ cycloalkyl.

In certain embodiments, Ring E is selected from cyclopropyl, cyclobutyl, cyclopentyl, or cyclohexyl.

In some embodiments, Ring E is aryl. In certain embodiments, Ring E is C₅₋₁₂ aryl. In certain embodiments, Ring E is C₅₋₁₁ aryl. In certain embodiments, Ring E is C₅₋₁₀ aryl. In certain embodiments, Ring E is C₅₋₉ aryl. In certain embodiments, Ring E is C₅₋₈ aryl. In certain embodiments, Ring E is C₅₋₇ aryl. In certain embodiments, Ring E is C₅₋₆ aryl.

In certain embodiments, Ring E is phenyl or naphthalenyl.

In some embodiments, Ring E is heteroaryl. In certain embodiments, Ring E is 5- to 12-membered heteroaryl. In certain embodiments, Ring E is 5- to 10-membered heteroaryl. In certain embodiments, Ring E is 5- to 8-membered heteroaryl. In certain embodiments, Ring E is 5- to 7-membered heteroaryl. In certain embodiments, Ring E is 5- to 6-membered heteroaryl.

In certain embodiments, Ring E is selected from the group consisting of pyridinyl, pyrimidinyl pyridazinyl, pyrazinyl, thienyl, thiazolyl, isothiazolyl, oxazolyl, isoxazolyl, imidazolyl, pyrazolyl, 1,2,3-triazolyl, 1,2,4-triazolyl, benzofuranyl, benzothienyl, indolyl, benzimidazolyl, benzopyrazolyl, purinyl, quinolinyl, isoquinolinyl, isoquinoline-1(2H)-one group, isoindolin-1-one group, benzo[d]oxazole-2(H)-one group and 1,3-dihydro-2H-benzo[d]imidazol-2-one group.

In some embodiments, T is N.

In some embodiments, T is C(R^{T}). In certain embodiments, R^{T} is hydrogen, cyano, fluoro, —CH₃, —OCH₃, or —CF₃.

In some embodiments, Y is O.

In some embodiments, Y is S.

In some embodiments, R⁴ is alkyl optionally substituted with one or more group independently selected from deuterium, hydroxyl, hydroxyalkyl, alkoxy, or —N(R′)₂. In certain embodiments, each R^{h} is independently hydrogen or alkyl (such as C₁₋₅ alkyl, C₁₋₄ alkyl, C₁₋₃ alkyl, or C₁₋₂ alkyl).

In some embodiments, R⁴ is selected from

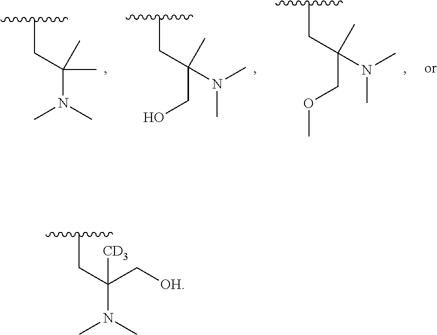

In a further aspect, the present disclosure provides a compound having Formula (IIa), Formula (IIb), Formula (IIc), Formula (IId), Formula (IIe) or Formula (IIf):

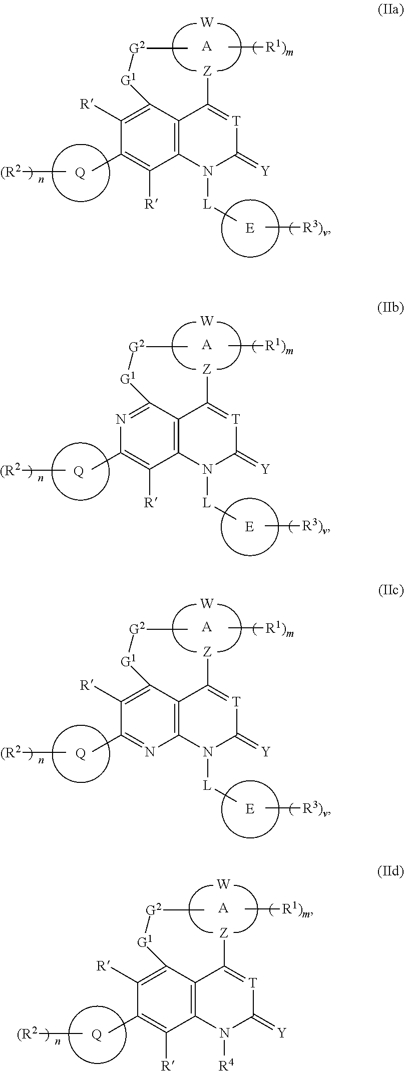

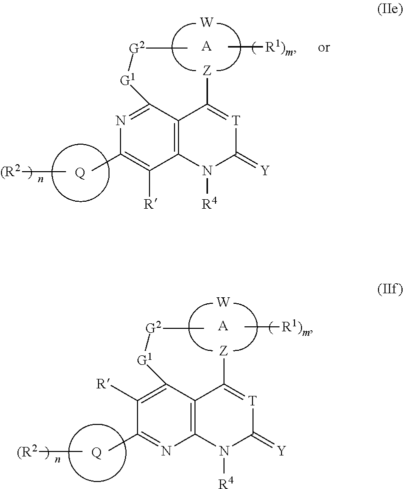

or a pharmaceutically acceptable salt thereof, wherein Ring A, Ring Q, Ring E, W, Z, G¹, G², Y, T, L, R¹, R², R³, R′, m, n and v are defined as supra.

In some embodiments, in Formula (IIa), Formula (IIb), Formula (IIc), Formula (IId), Formula (IIe) or Formula (IIf), Ring Q is naphthalenyl, benzothiophenyl or pyridinyl.

In some embodiments, in Formula (IIa), Formula (IIb), Formula (IIc), Formula (IId), Formula (IIe) or Formula (IIf), G¹ is —O—.

In some embodiments, in Formula (IIa), Formula (IIb), Formula (IIc), Formula (IId), Formula (IIe) or Formula (IIf), G² is —[C(R^{d})₂]ᵤ—, and each R^{d} is independently hydrogen or alkyl optionally substituted with one or more deuterium. In certain embodiments, u is 1 or 2. In certain embodiments, G² is —CH₂—.

In some embodiments, in Formula (IIa), Formula (IIb), Formula (IIc), Formula (IId), Formula (IIe) or Formula (IIf), G¹ is —O—, G² is —[C(R^{d})₂]ᵤ—, each R^{d} is independently hydrogen or alkyl optionally substituted with one or more deuterium, and u is 1 or 2. In certain embodiments, each R^{d} is independently hydrogen, C₁₋₆ alkyl, C₁₋₅ alkyl, C₁₋₄ alkyl, or C₁₋₃ alkyl, wherein each alkyl is optionally substituted with one or more deuterium. In certain embodiments, each R^{d} is independently hydrogen or methyl optionally substituted with one or more deuterium. In certain embodiments, -G¹-G²- is —O—CH₂—, —O—CH(CH₃)— or —O—CH(CD₃)—.

In some embodiments, in Formula (IIa), Formula (IIb), Formula (IIc), Formula (IId), Formula (IIe) or Formula (IIf), G¹ is —[C(R^{d})₂]ᵤ—, and each R^{d} is independently hydrogen or alkyl optionally substituted with one or more deuterium. In certain embodiments, u is 1 or 2. In certain embodiments, G1 is —CH₂—.

In some embodiments, in Formula (IIa), Formula (IIb), Formula (IIc), Formula (IId), Formula (IIe) or Formula (IIf), G² is —N(R^{c})—, and R^{c} is hydrogen or alkyl optionally substituted with one or more deuterium.

In some embodiments, in Formula (IIa), Formula (IIb), Formula (IIc), Formula (IId), Formula (IIe) or Formula (IIf), G¹ is —[C(R^{d})₂]ᵤ—, G² is —N(R^{c})—. In certain embodiments, each of R^{c} and R^{d} is independently hydrogen or alkyl optionally substituted with one or more deuterium, and u is 1 or 2. In certain embodiments, each of R^{c} and R^{d} is independently hydrogen, C₁₋₆ alkyl, C₁₋₅ alkyl, C₁₋₄ alkyl, or C₁₋₃ alkyl, wherein each alkyl is optionally substituted with one or more deuterium. In certain embodiments, each of R^{c} and R^{d} is independently hydrogen or methyl optionally substituted with one or more deuterium. In certain embodiments, -G¹-G²- is —CH₂—N(CH₃)—.

In some embodiments, in Formula (IIa), Formula (IIb), Formula (IIc), Formula (IId), Formula (IIe) or Formula (IIf),

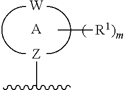

is selected from

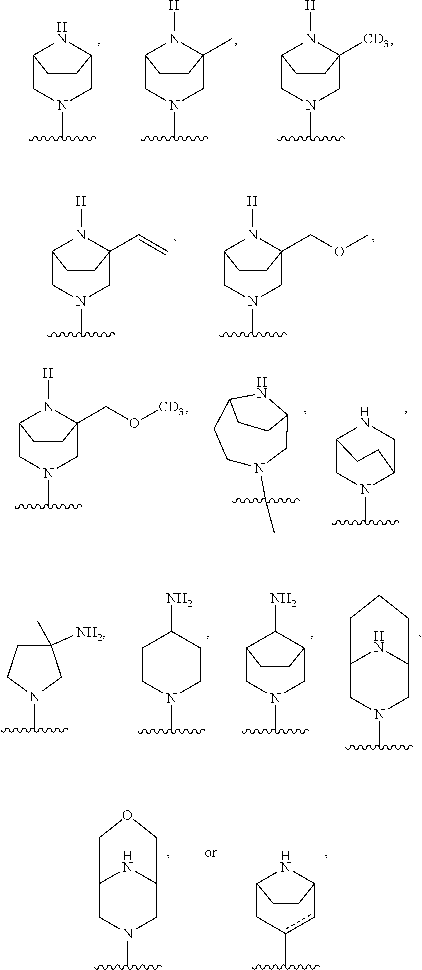

and [symbol] represents a single bond or a double bond.

In some embodiments, in Formula (IIa), Formula (IIb), Formula (IIc), Formula (IId), Formula (IIe) or Formula (IIf), R³ is selected from hydrogen, halogen, alkyl, alkoxy, hydroxyalkyl, heterocyclyl, —N(R^{g})₂ or ═C(R″)₂, wherein the alkyl, alkoxy, hydroxyalkyl, and heterocyclyl are optionally substituted with one or more groups selected from deuterium.

In some embodiments, in Formula (IIa), Formula (IIb), Formula (IIc), Formula (IId), Formula (IIe) or Formula (IIf), v is an integer from 1 to 4, each R³ is independently selected from F, —CH₃, —CD₃, ═CH₂, ═CHF, ═CF₂, —CH₂OH, —OCH₃, —N(CH₃)₂, or

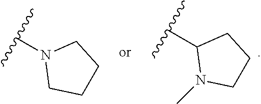

In some embodiments, in Formula (IIa), Formula (IIb), Formula (IIc), Formula (IId), Formula (IIe) or Formula (IIf), v is an integer from 1 to 4, and at least one R³ is ═C(R″)₂.

In some embodiments, in Formula (IIa), Formula (IIb), Formula (IIc), Formula (IId), Formula (IIe) or Formula (IIf), v is an integer from 1 to 4, at least one R³ is ═CH₂, ═CHF, or ═CF₂.

In some embodiments, in Formula (IIa), Formula (IIb), Formula (IIc), Formula (IId), Formula (IIe) or Formula (IIf), L is

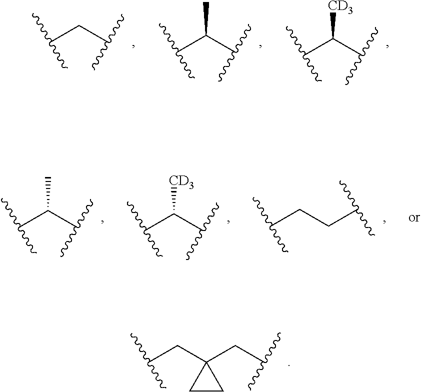

In a further aspect, the present disclosure provides a compound having Formula (IIIa), Formula (IIIb) or Formula (IIIc):

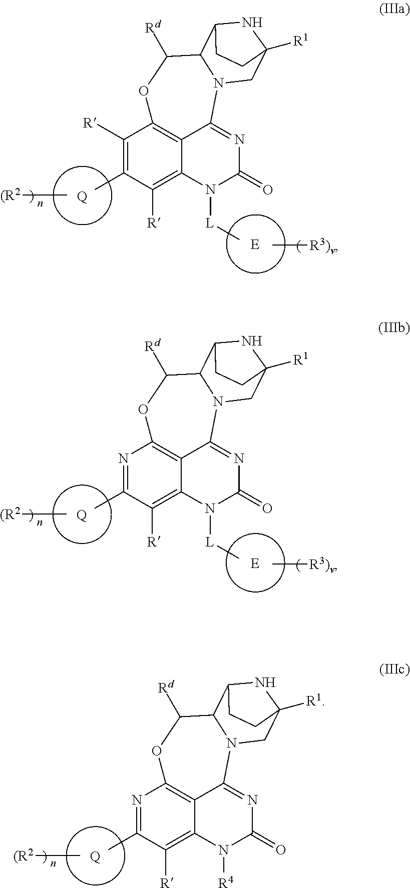

or a pharmaceutically acceptable salt thereof, wherein Ring Q, Ring E, L, R¹, R², R³, R⁴, R′, R^{d}, n and v are defined as supra.

In some embodiments, the present disclosure provides a compound having a formula selected from the group consisting of:

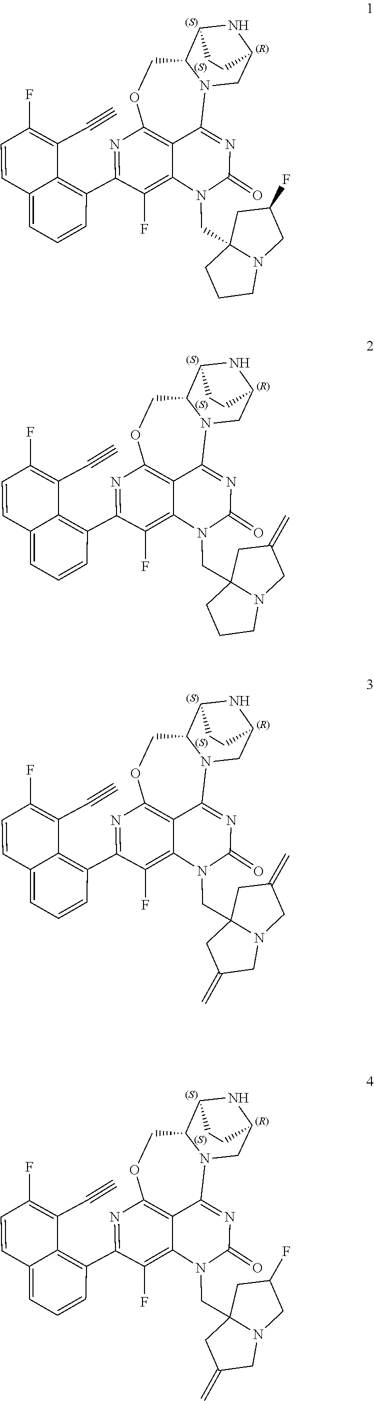

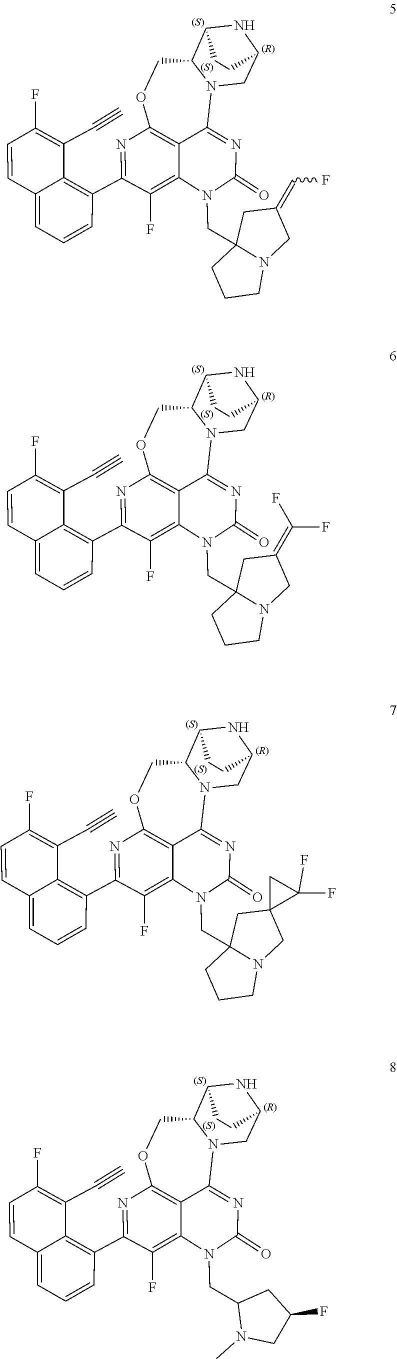

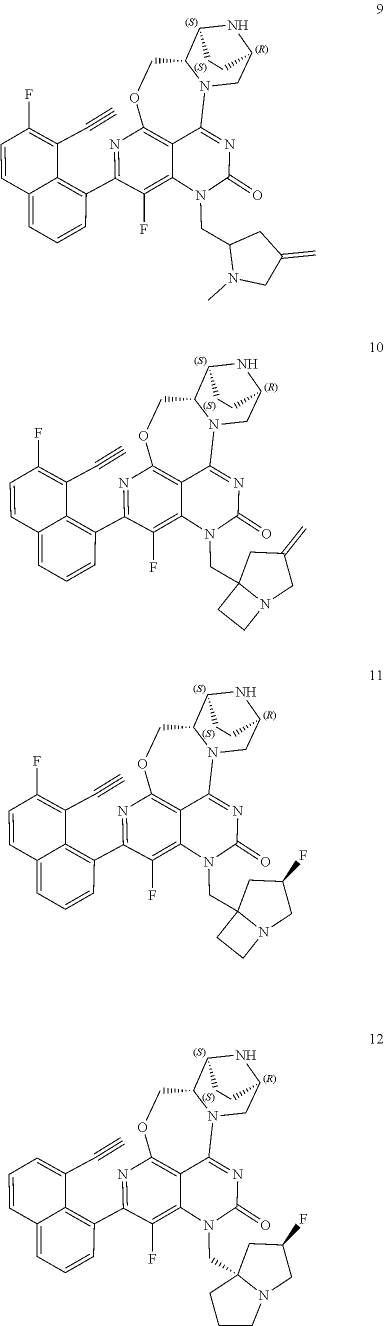

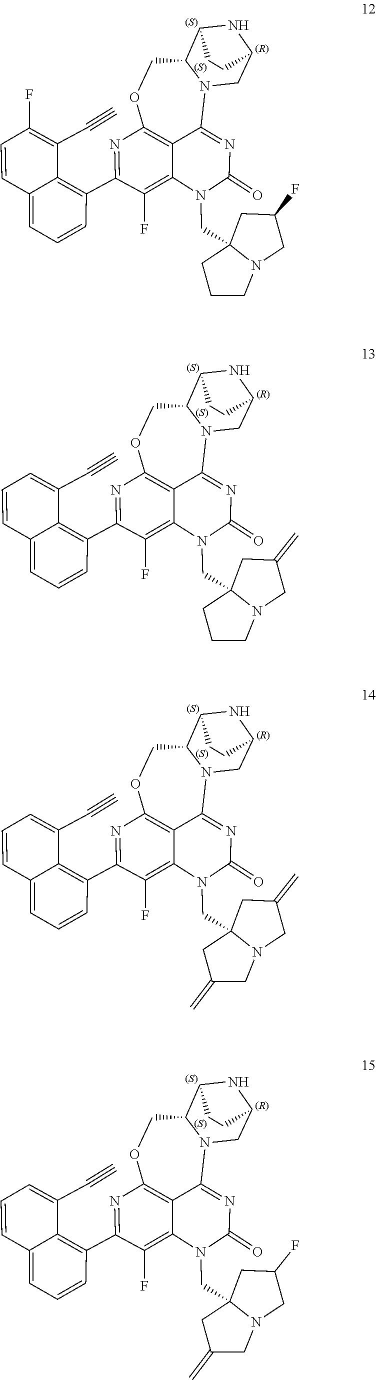

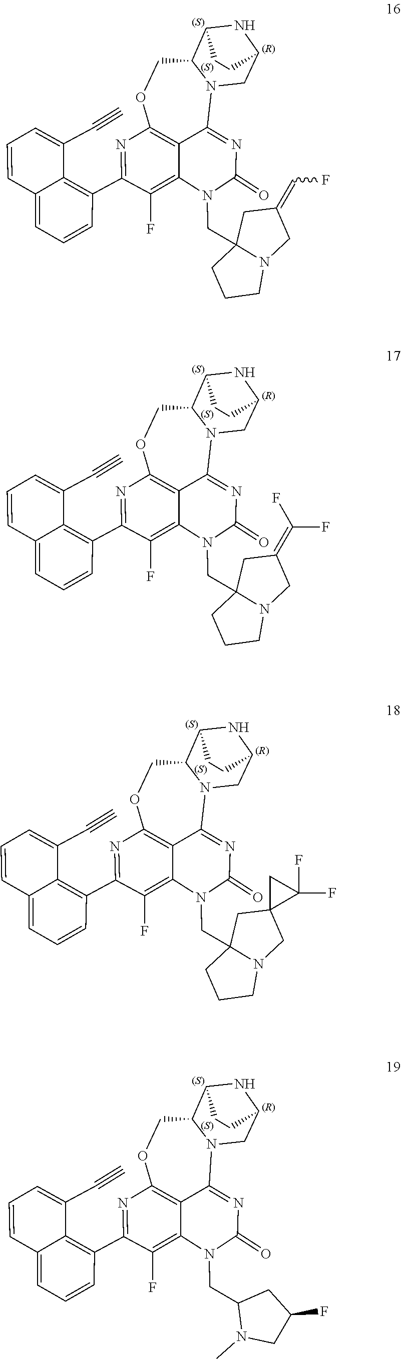

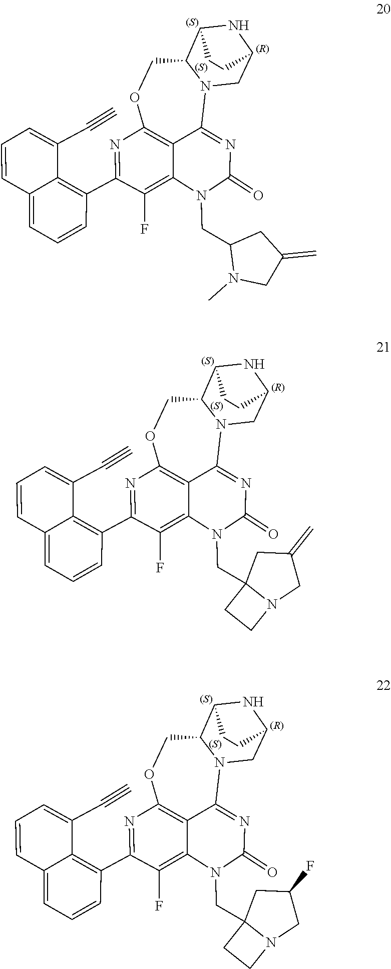

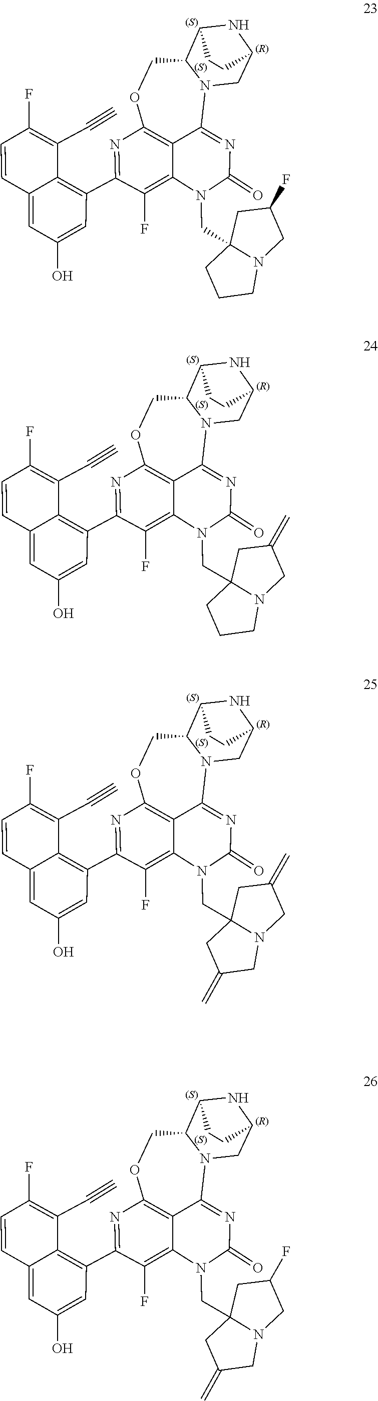

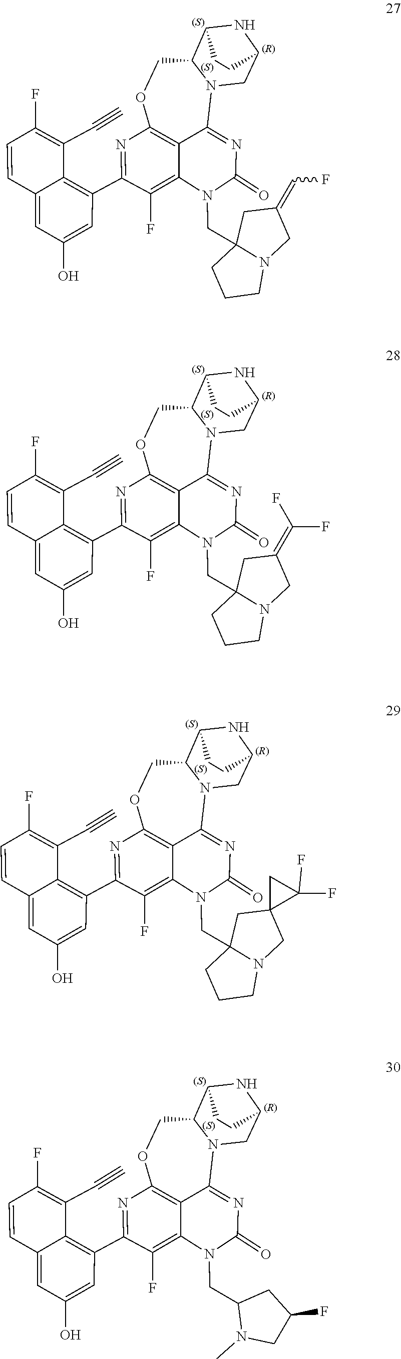

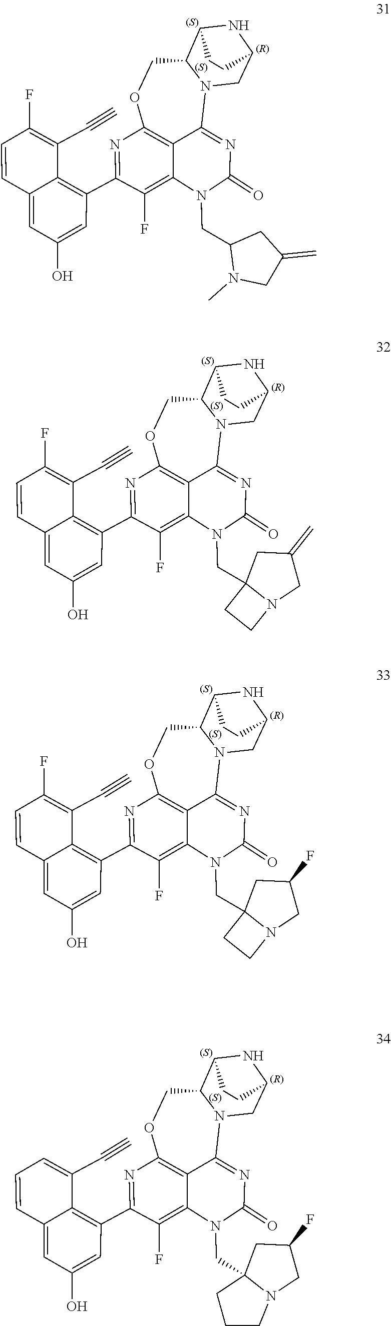

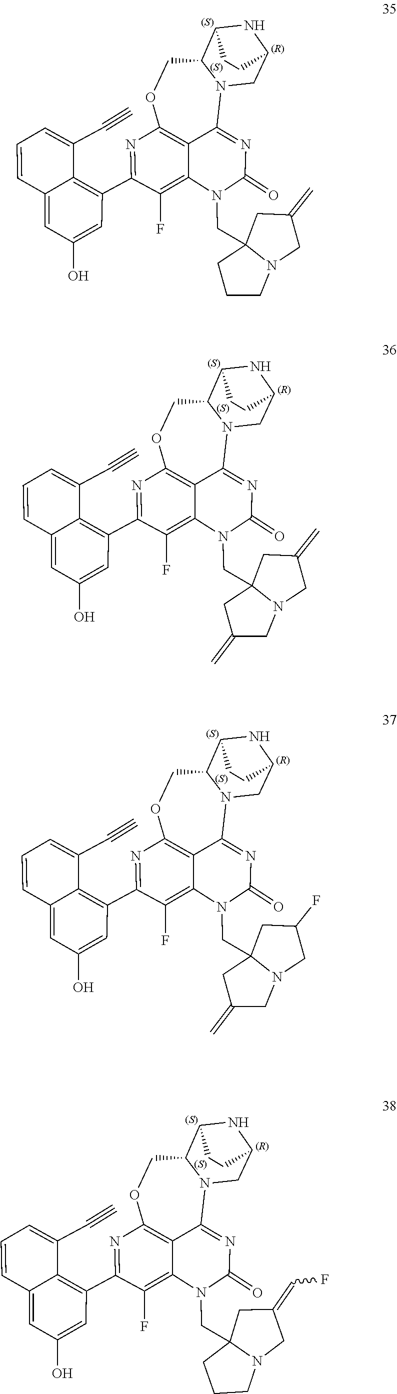

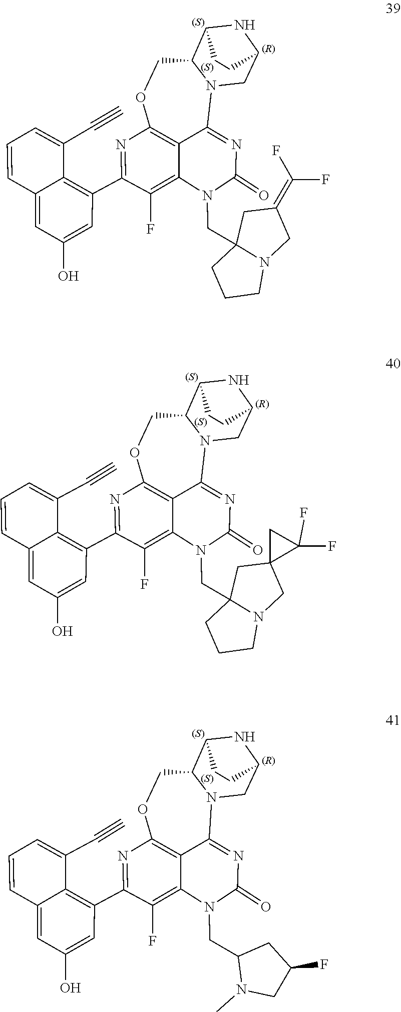

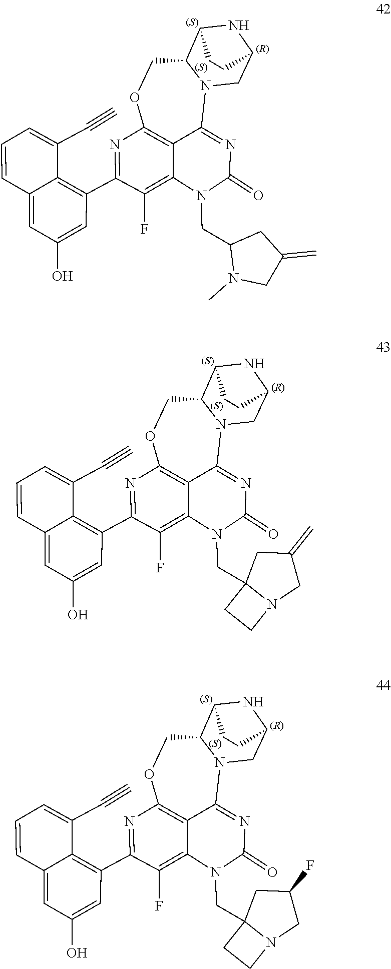

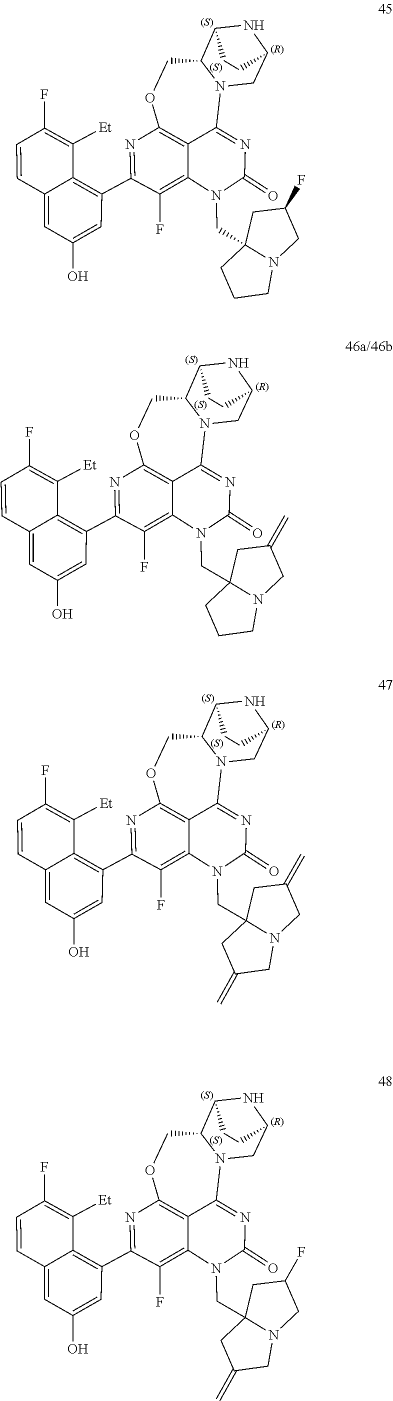

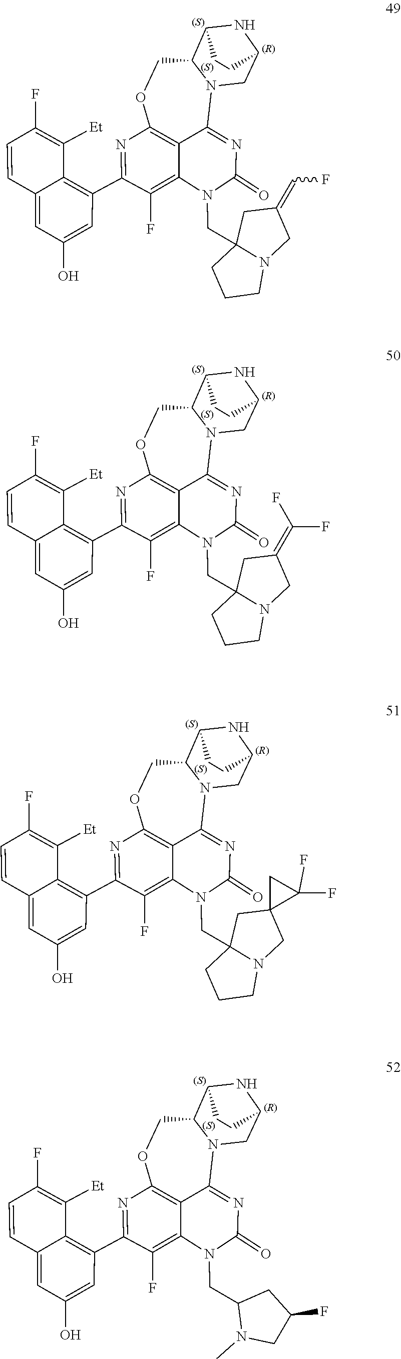

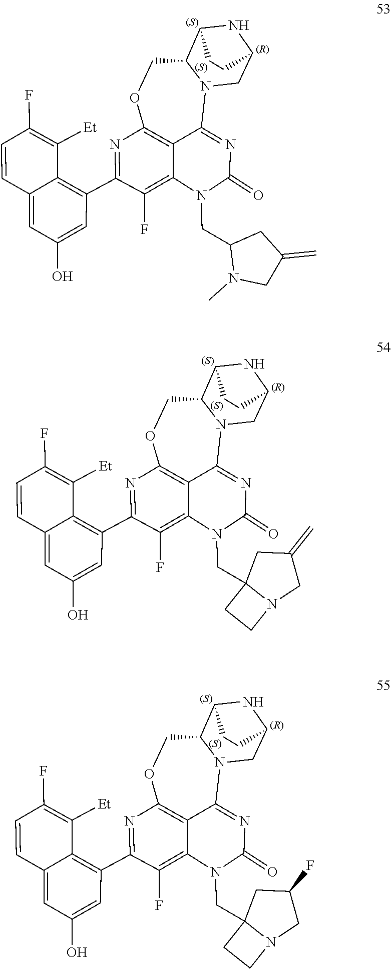

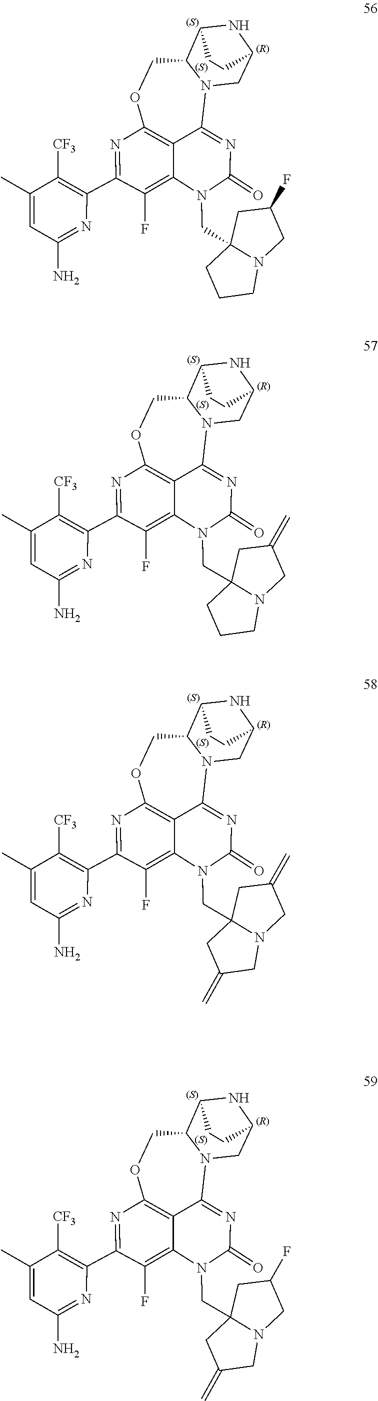

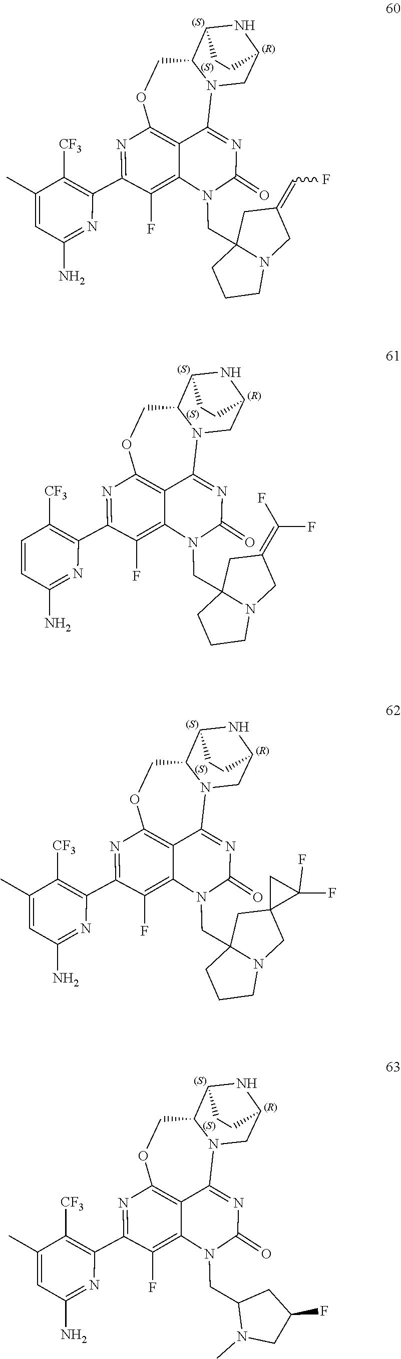

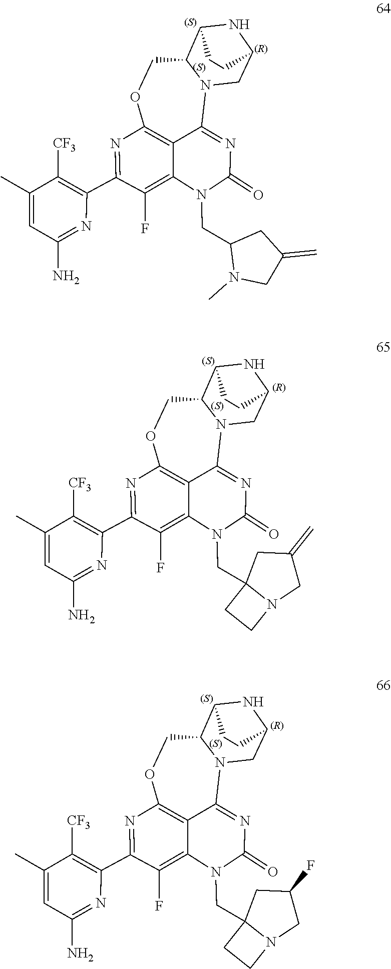

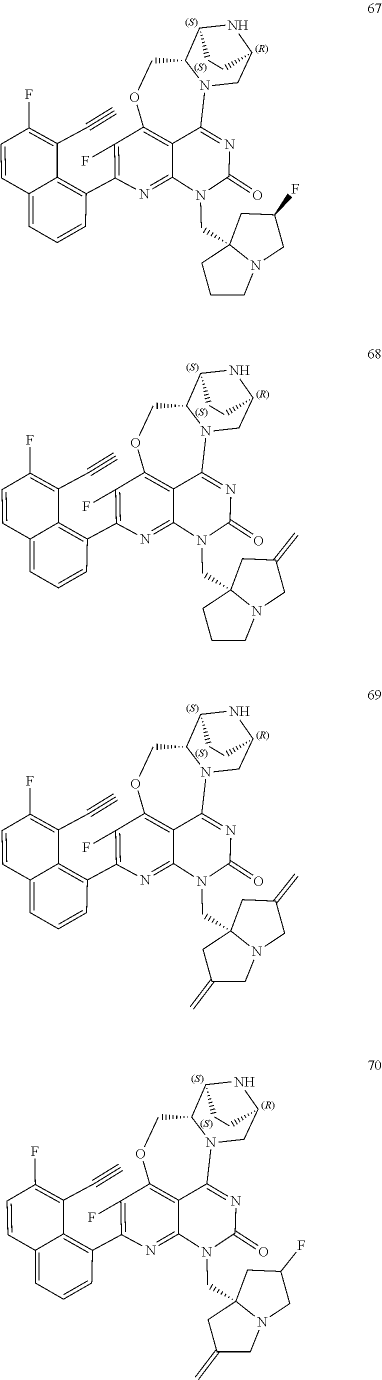

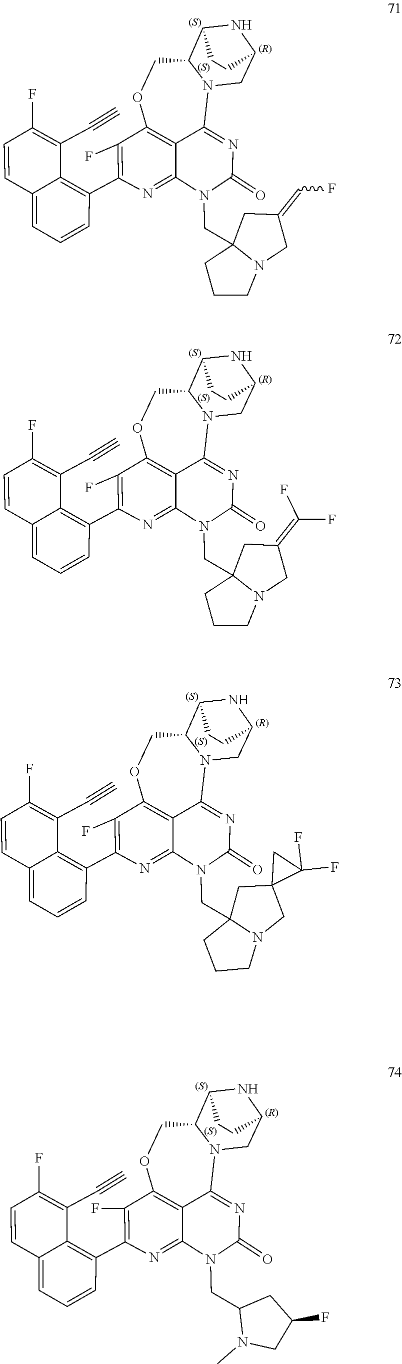

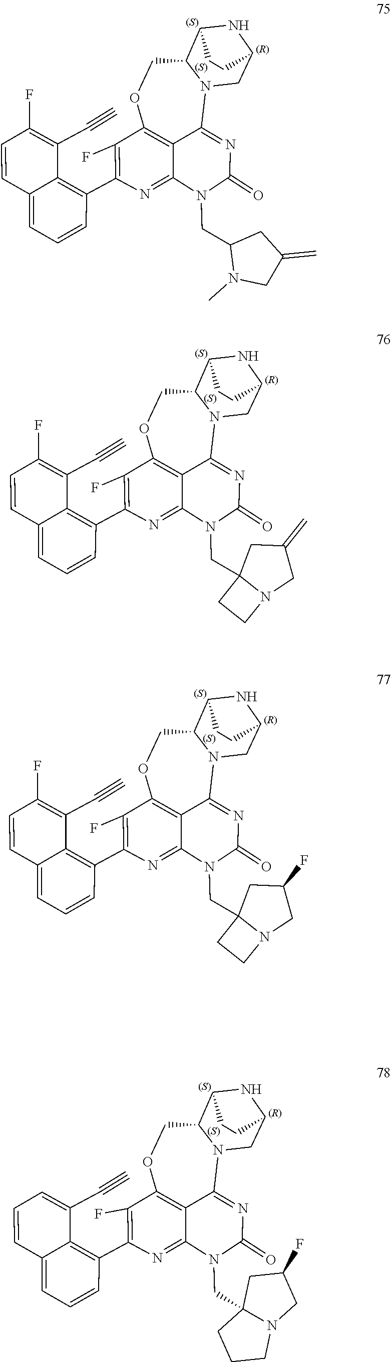

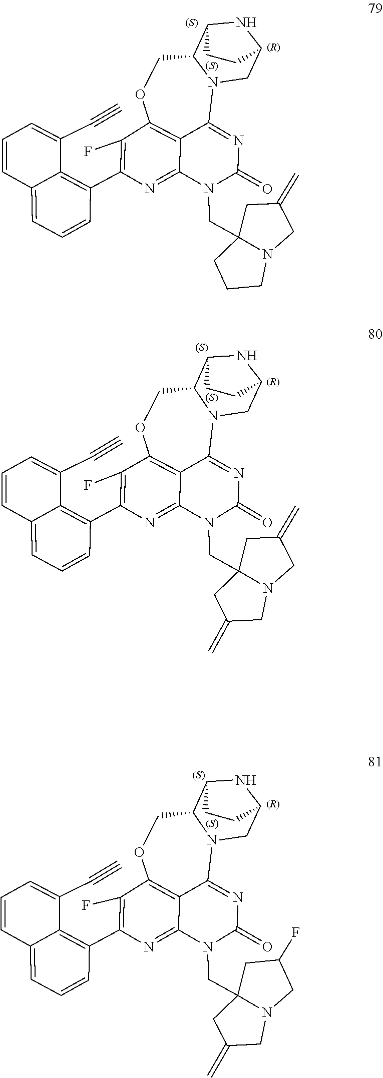

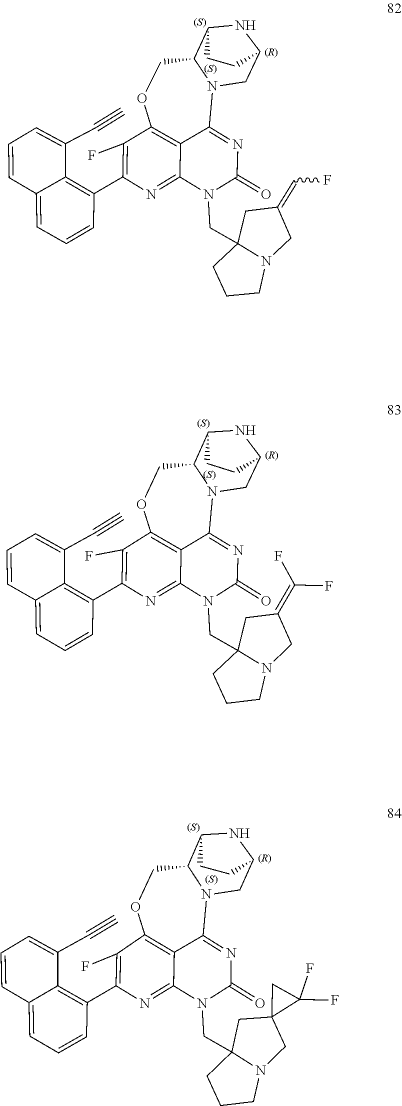

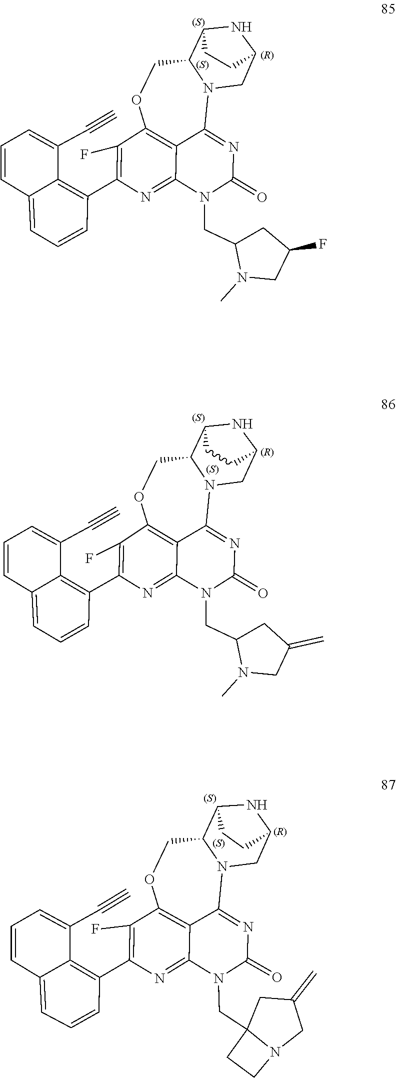

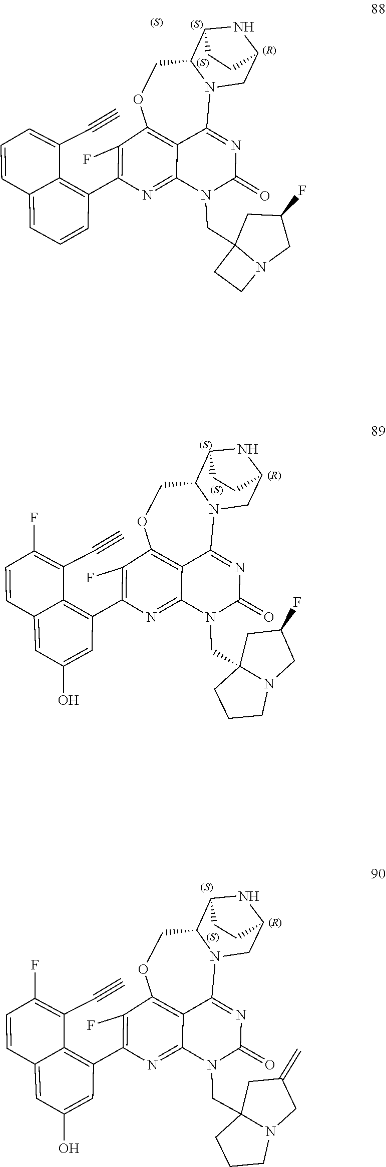

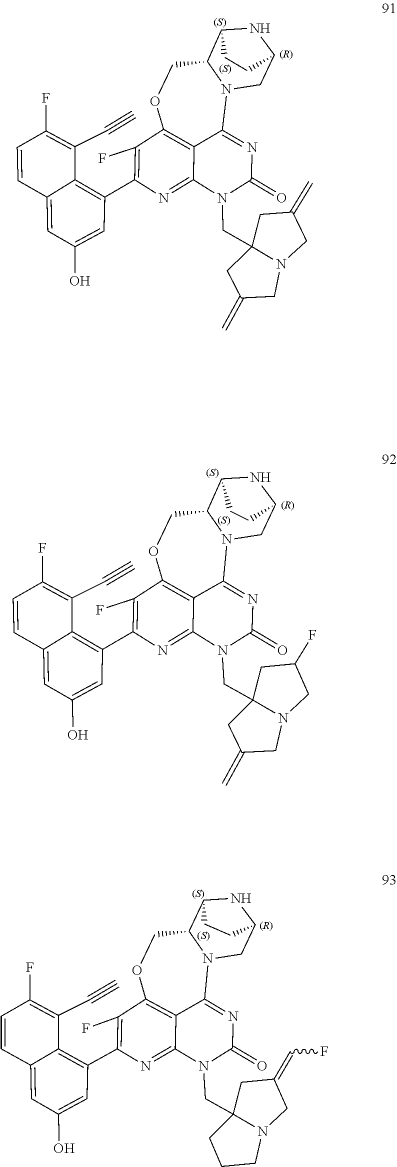

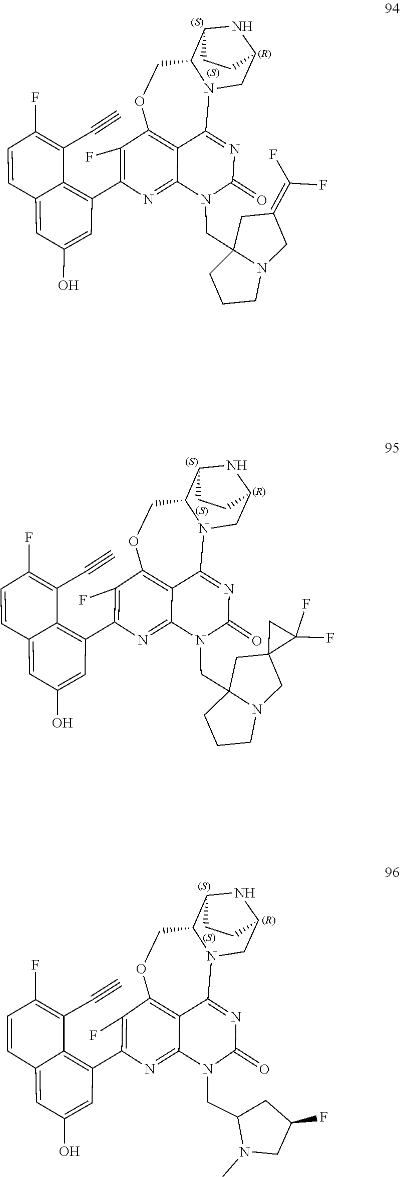

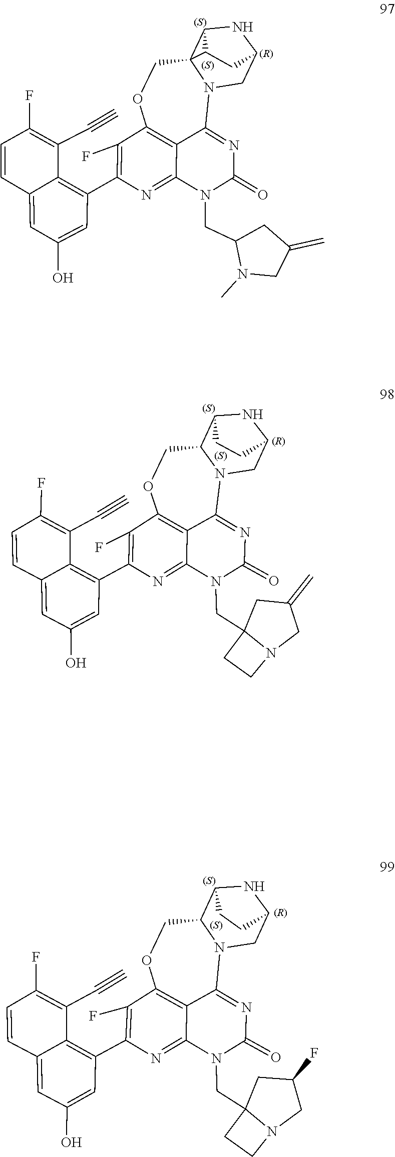

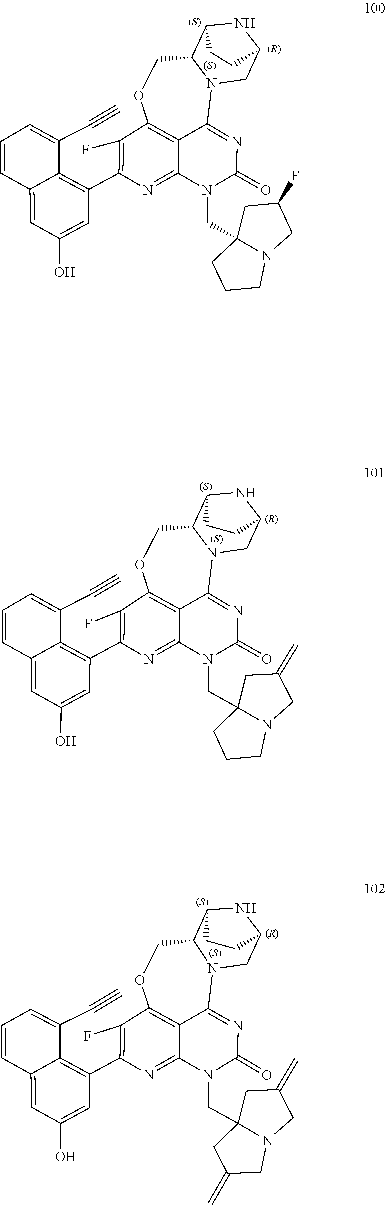

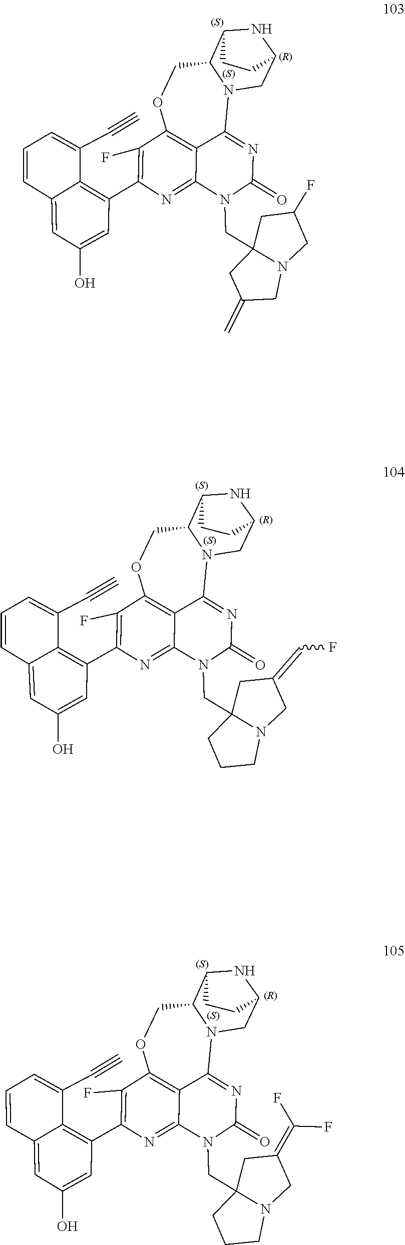

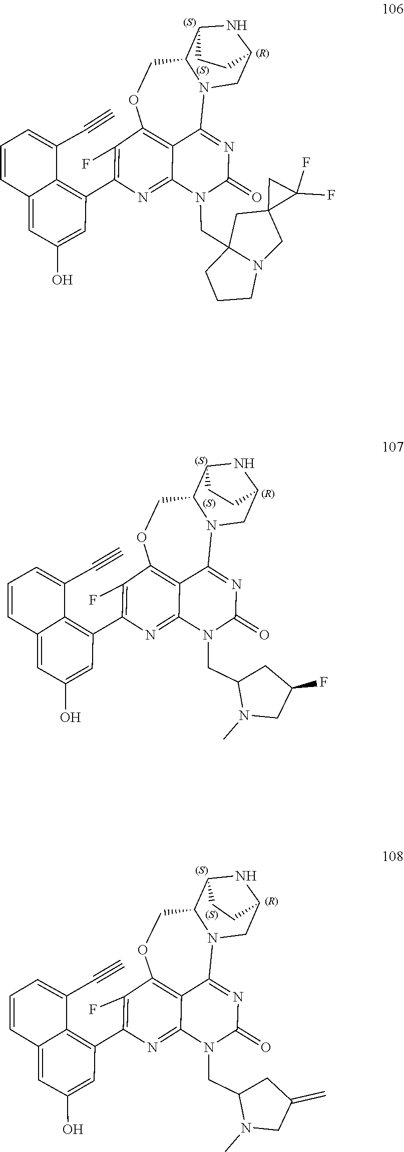

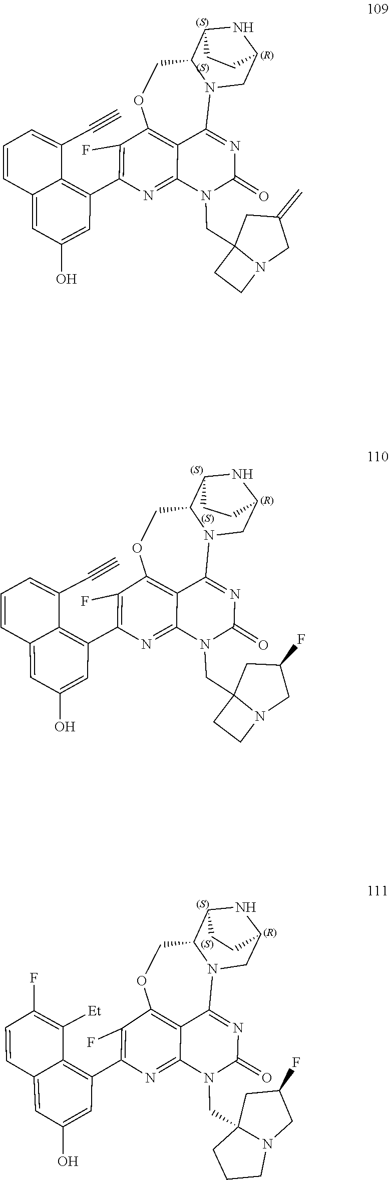

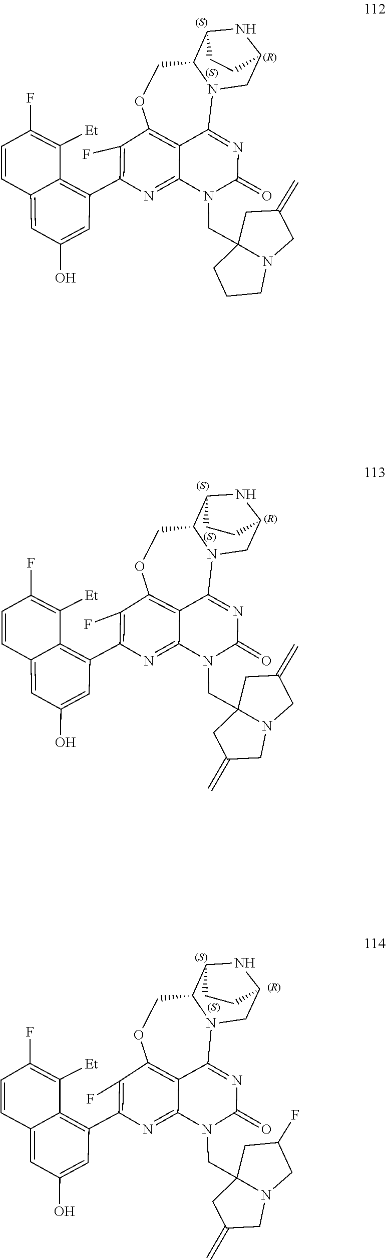

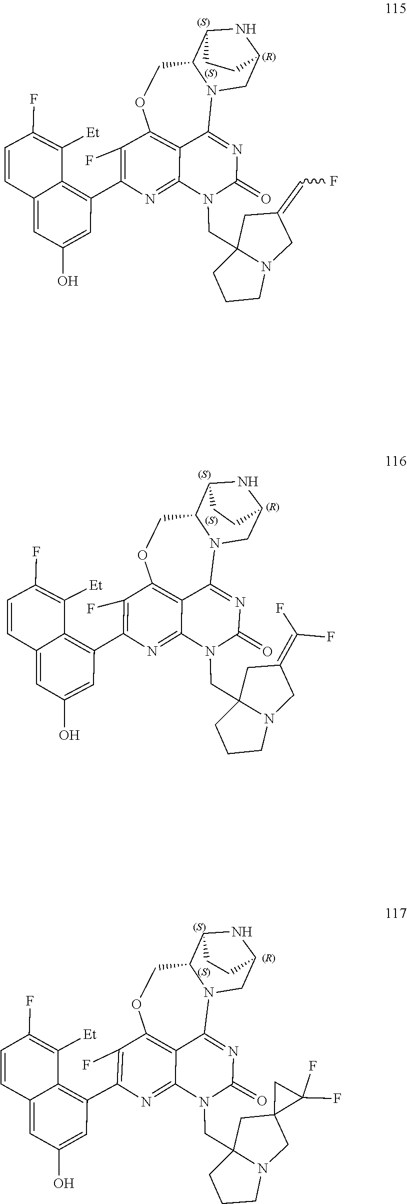

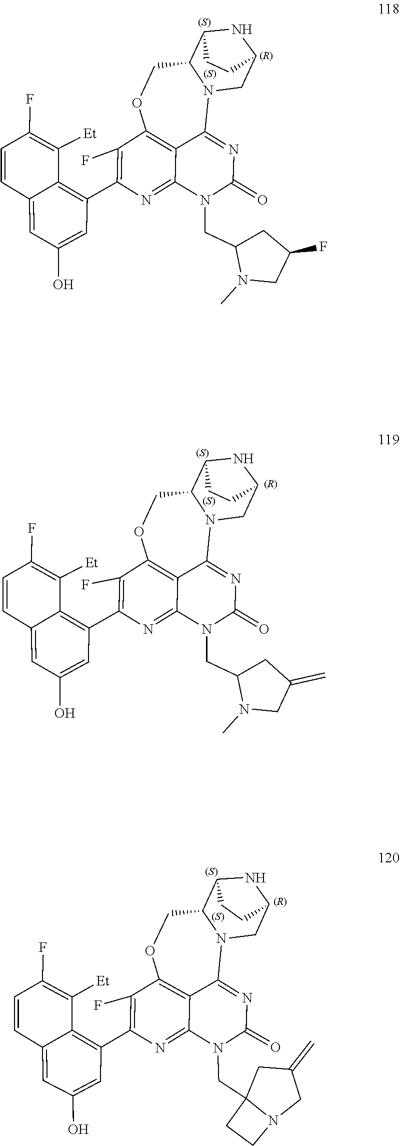

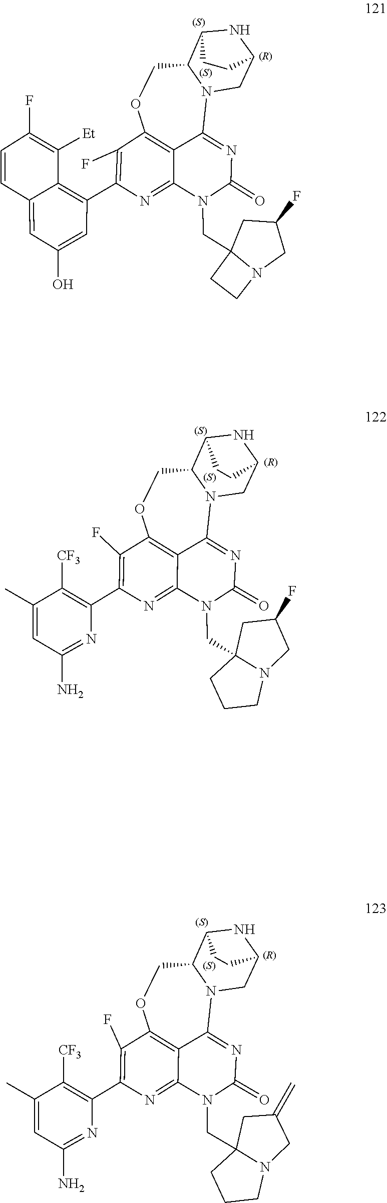

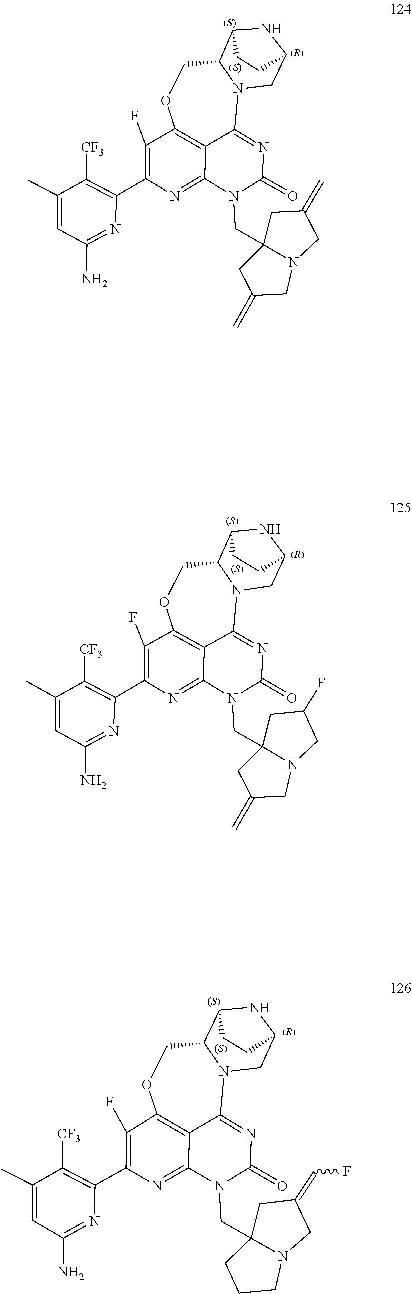

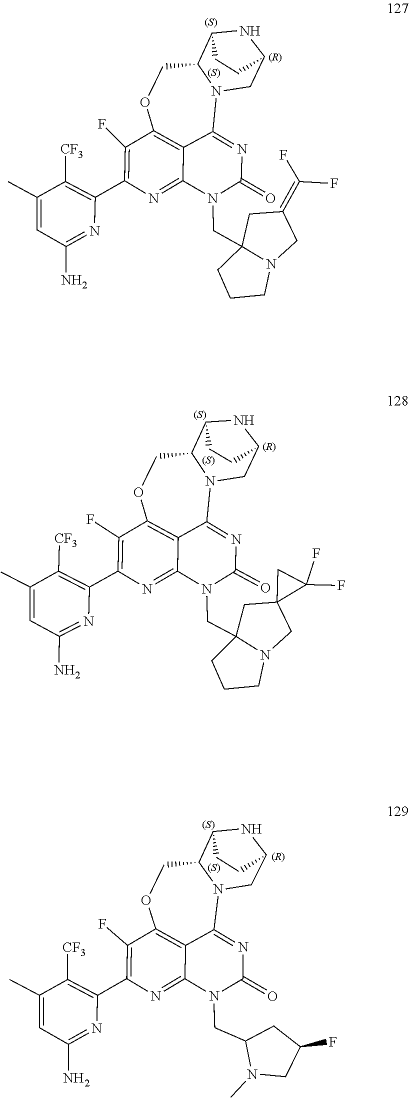

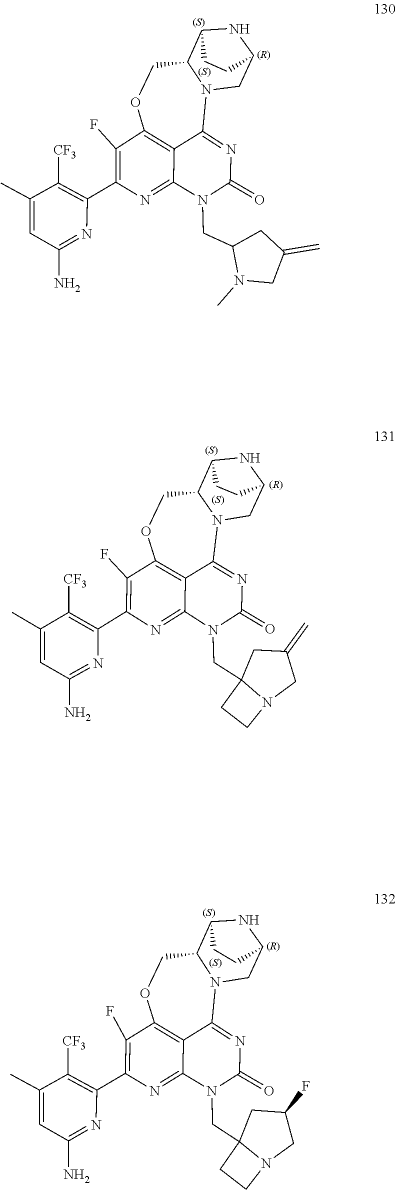

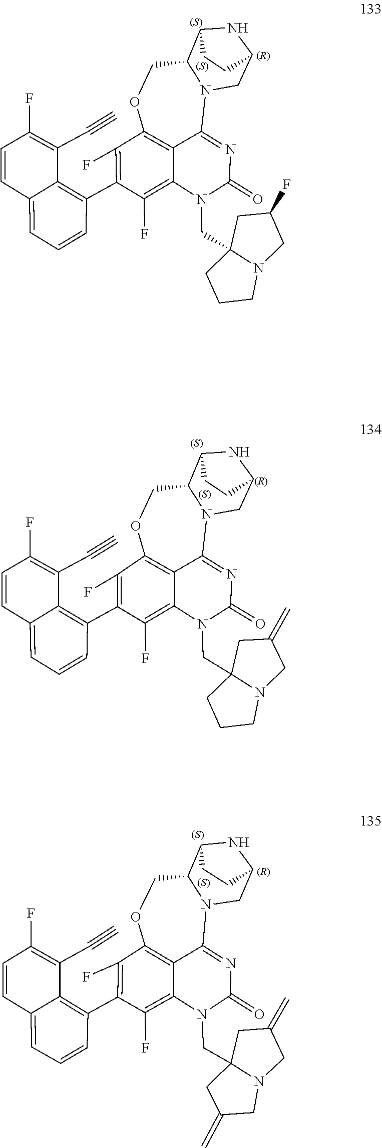

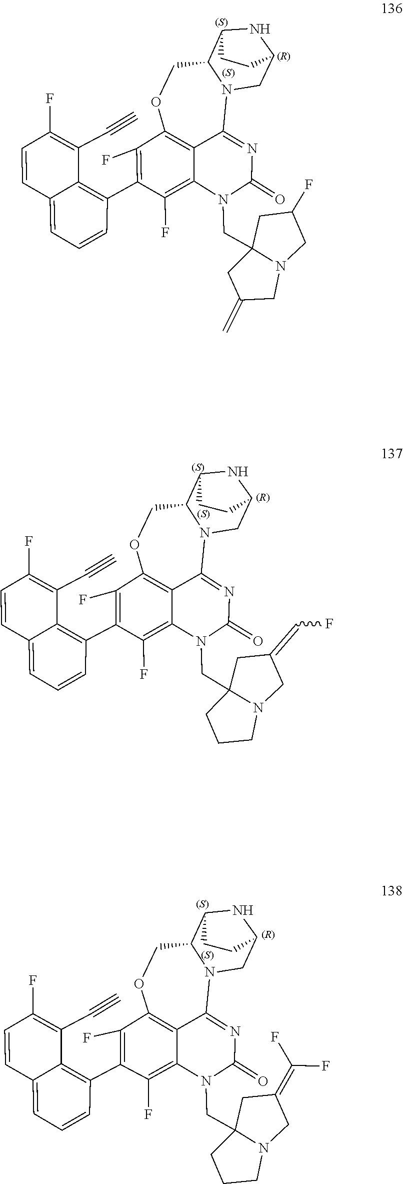

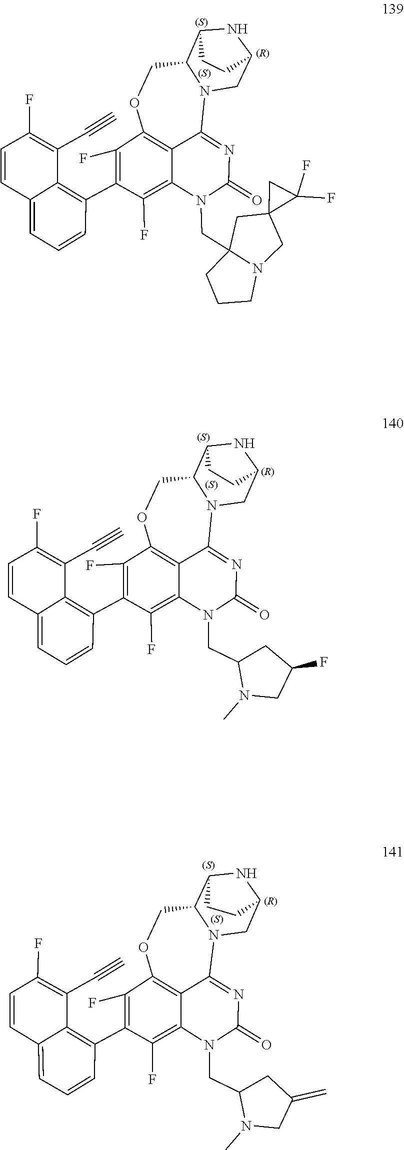

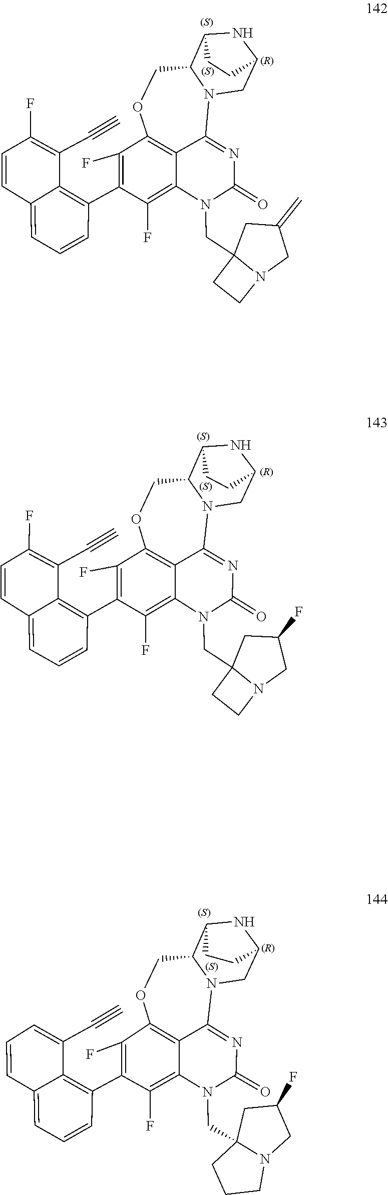

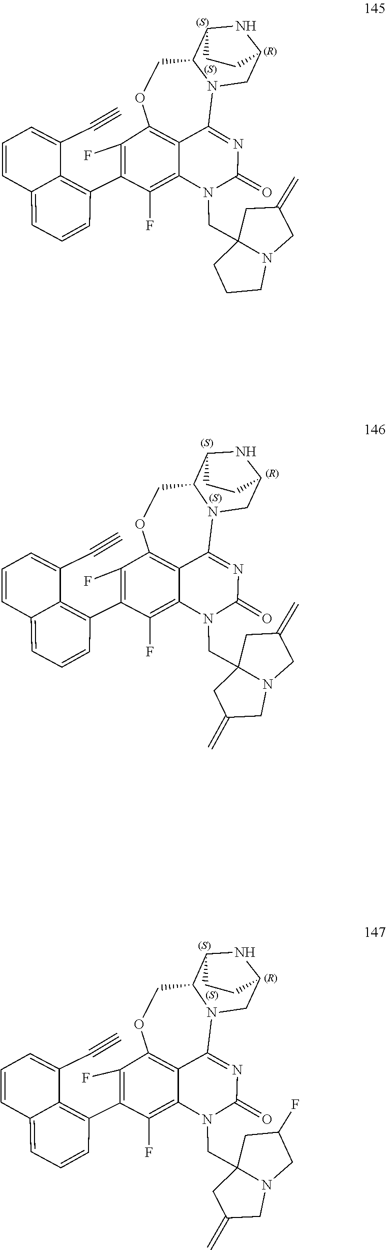

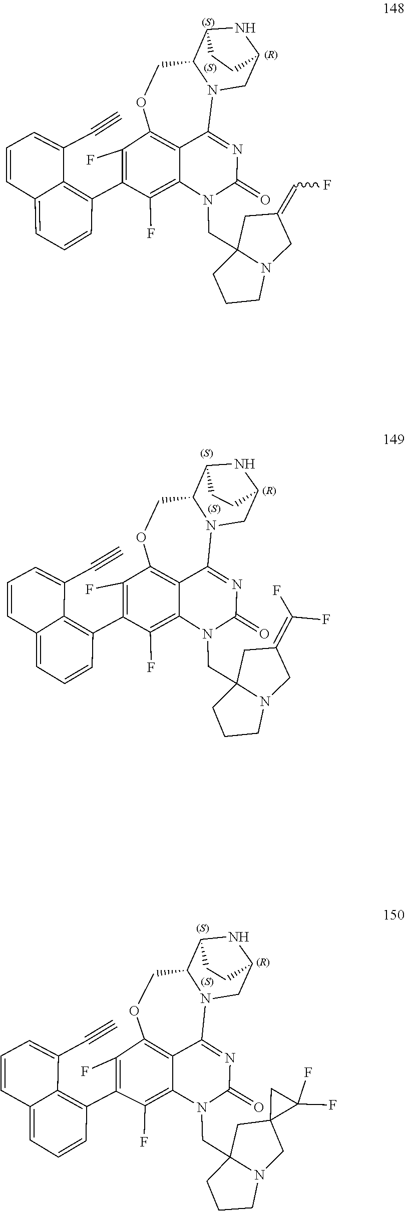

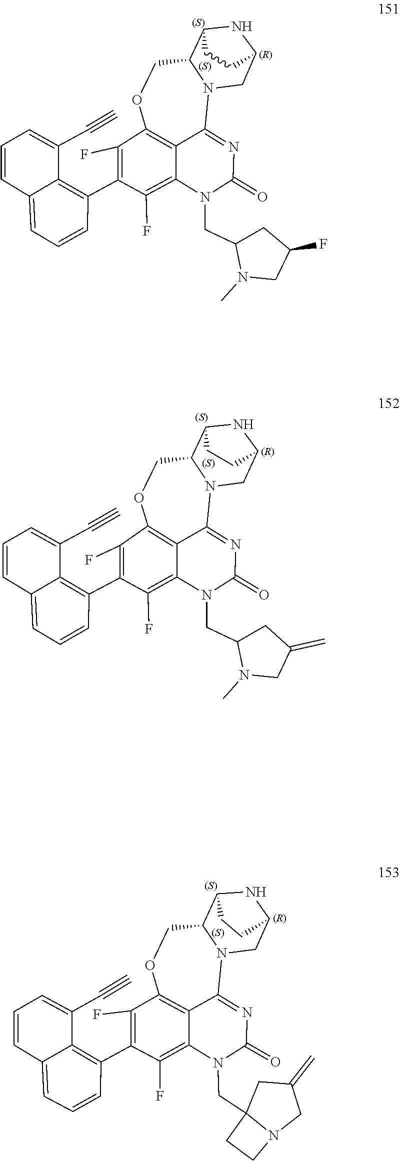

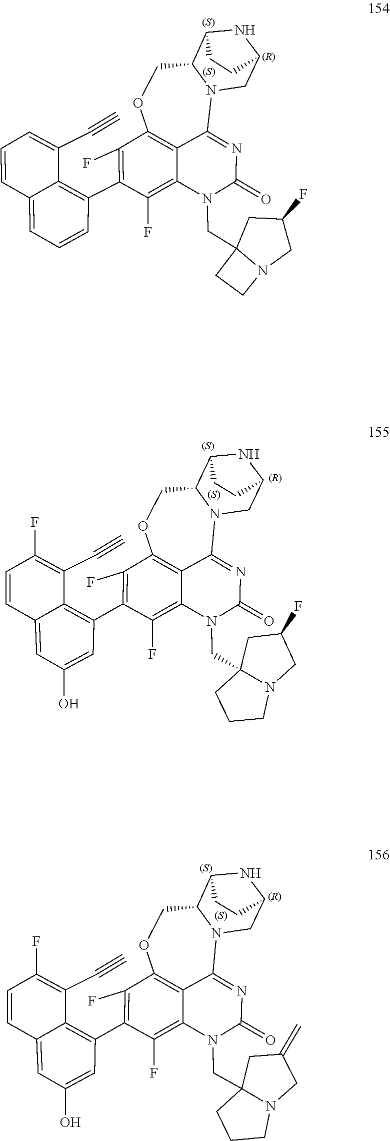

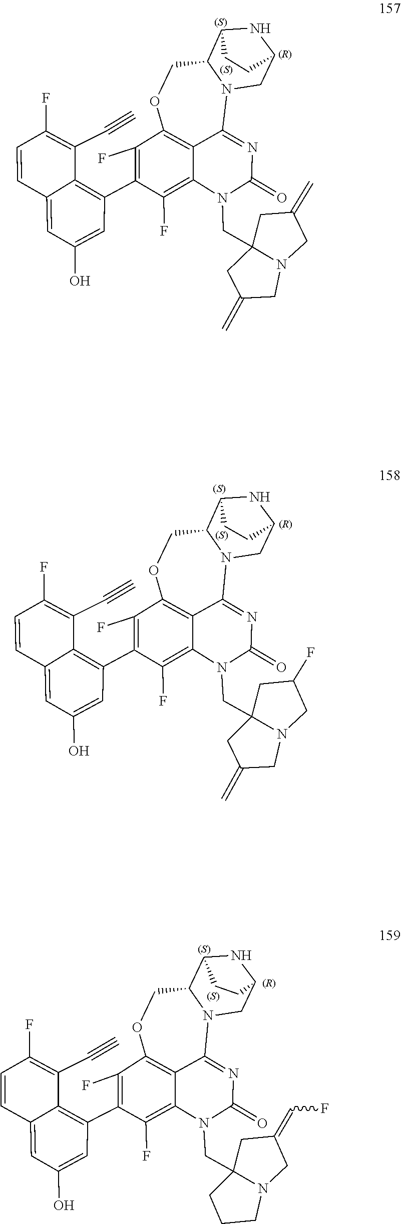

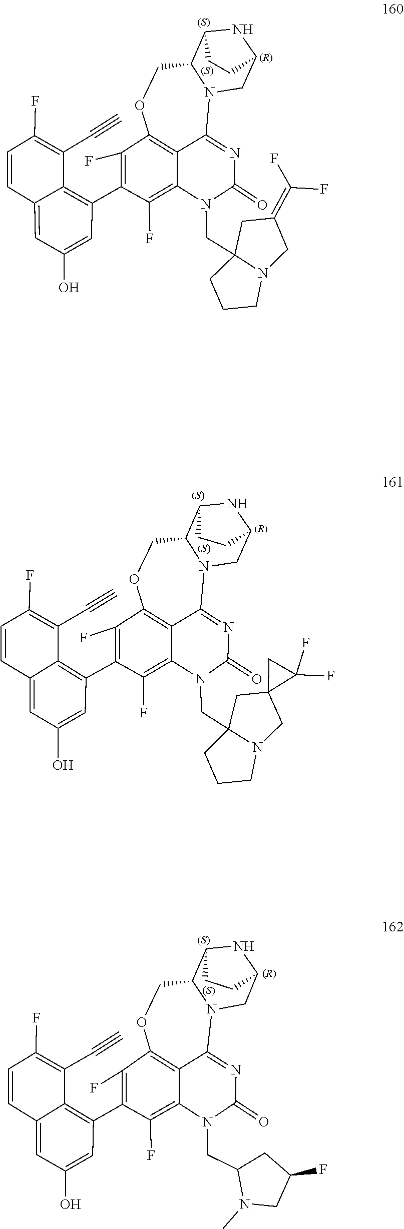

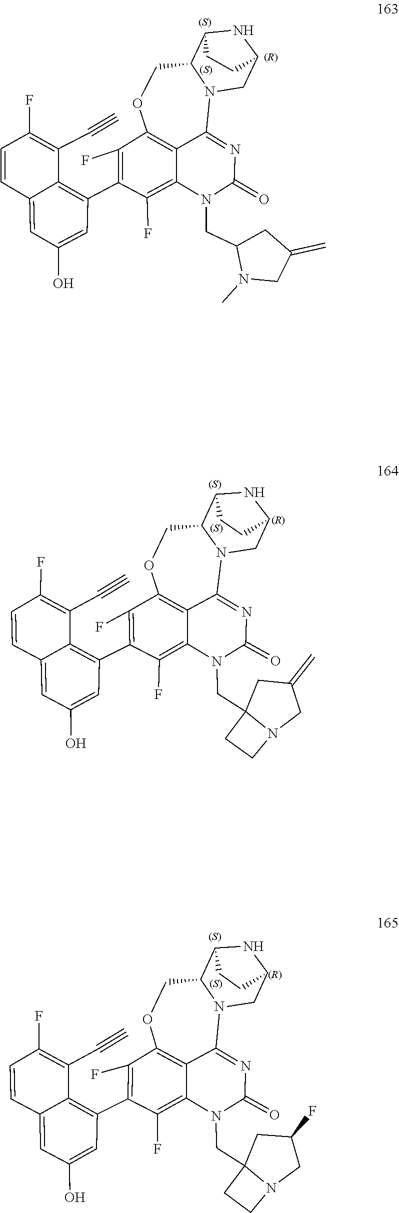

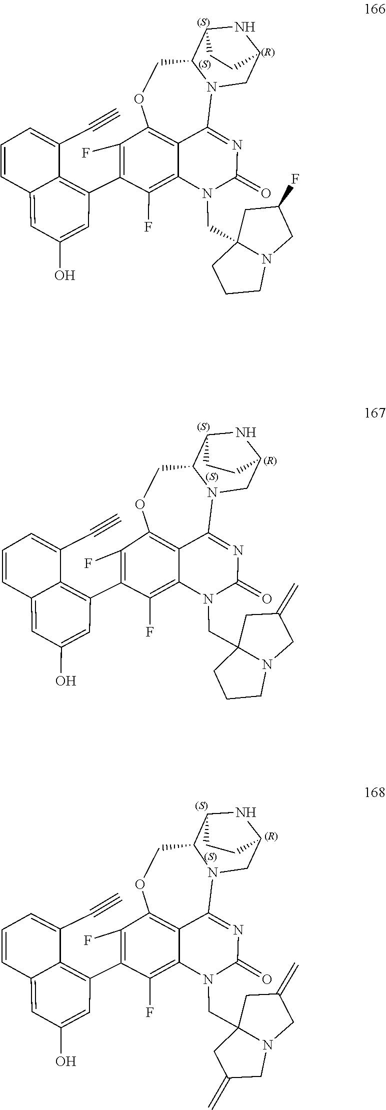

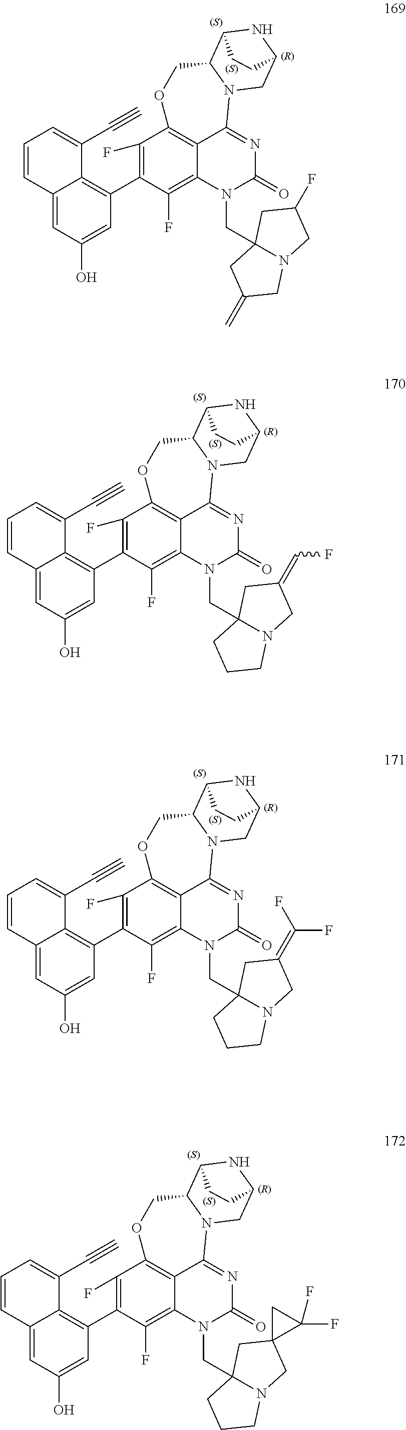

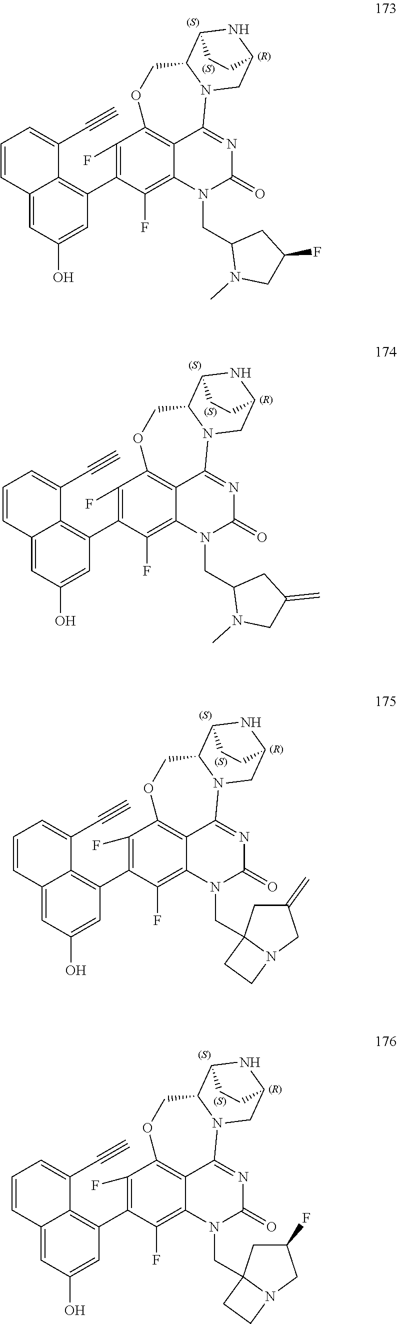

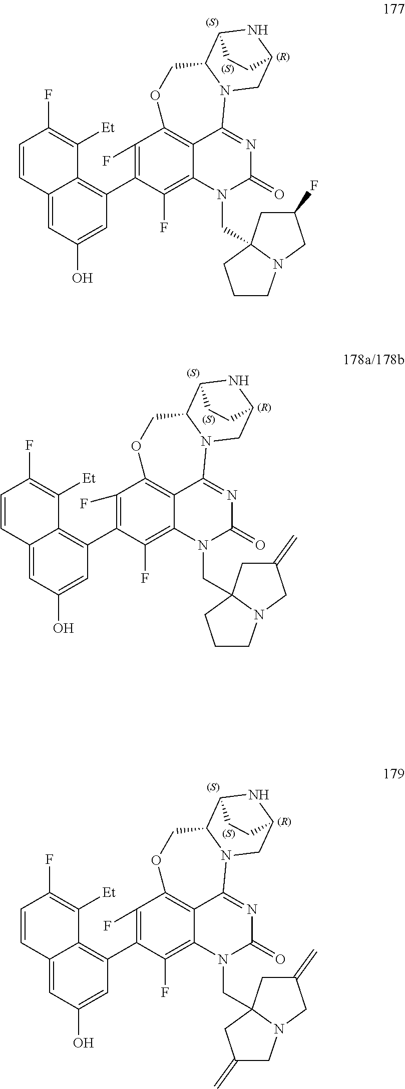

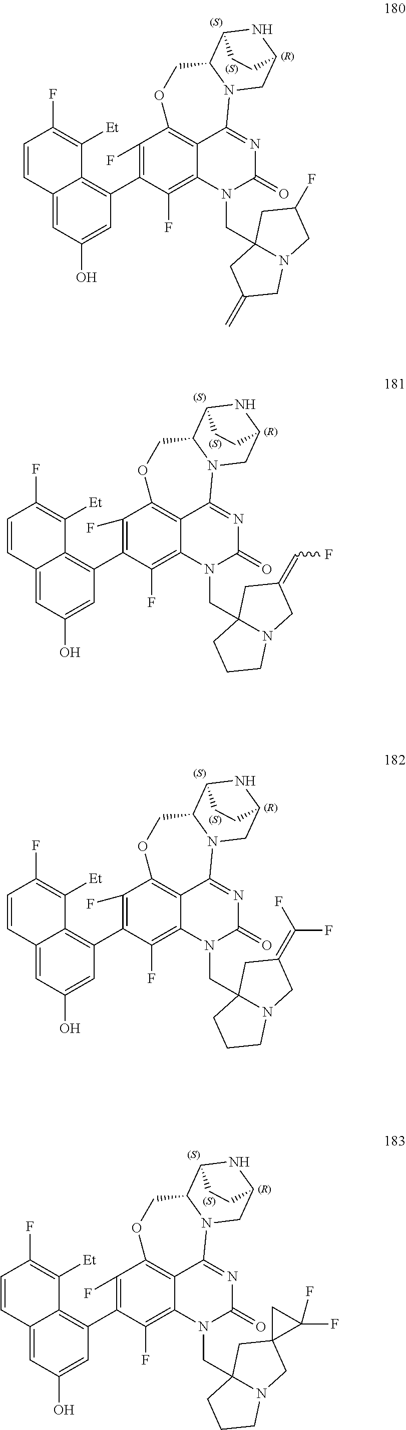

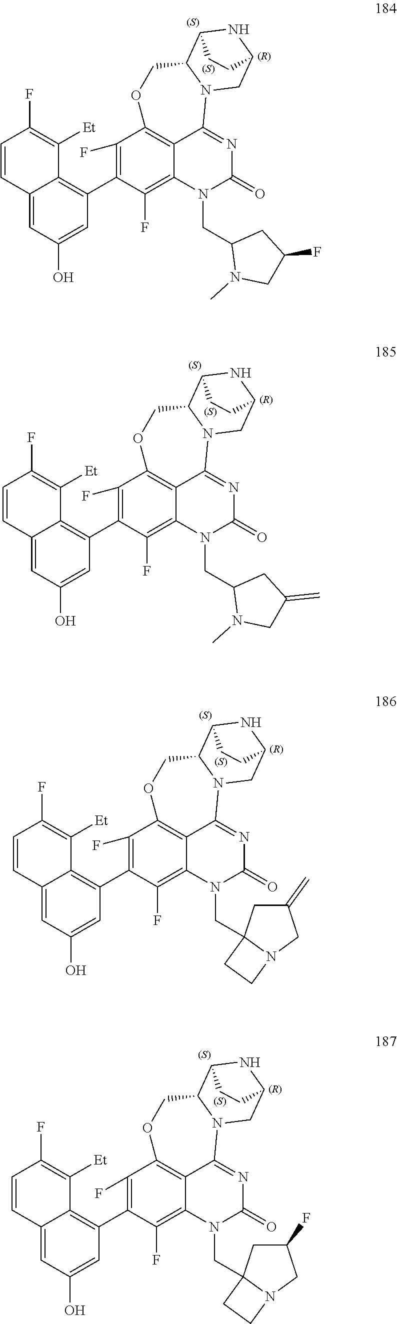

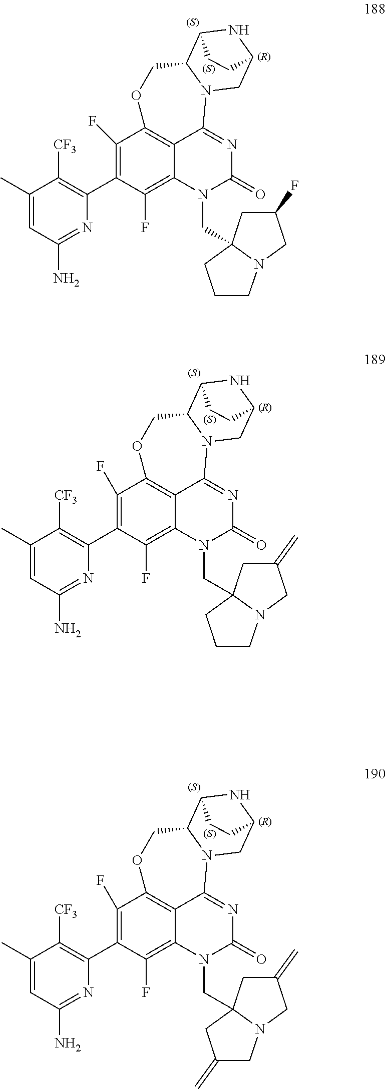

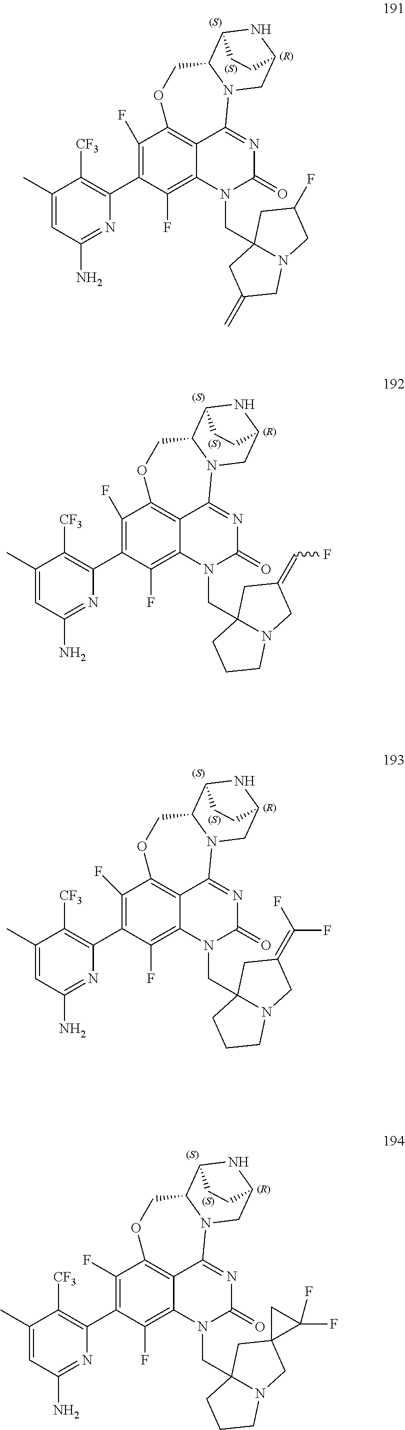

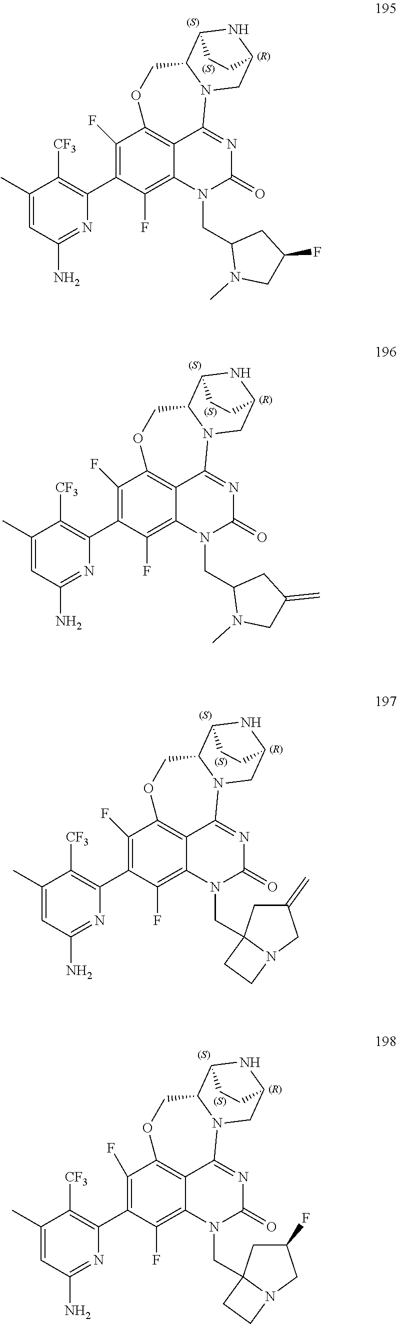

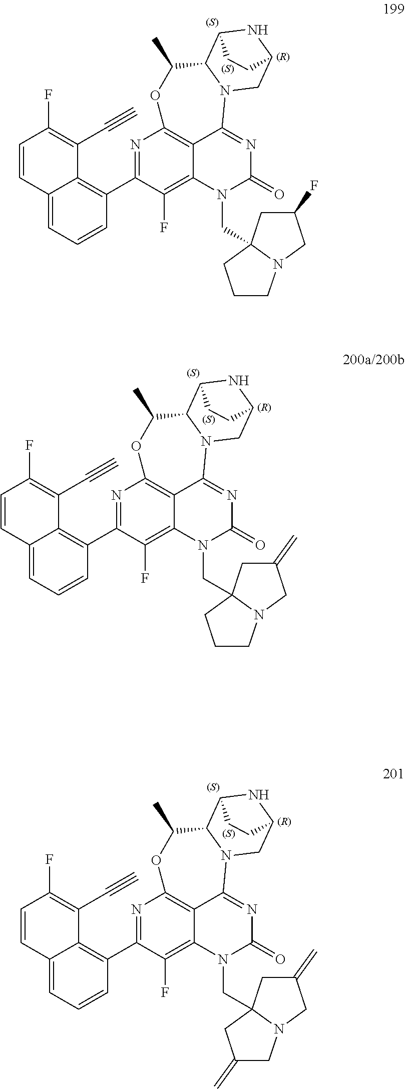

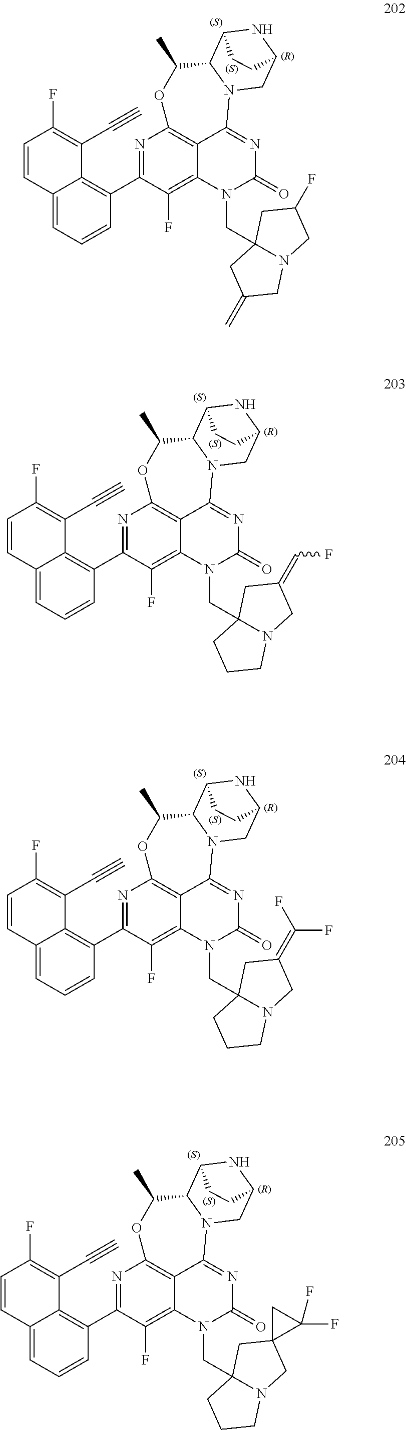

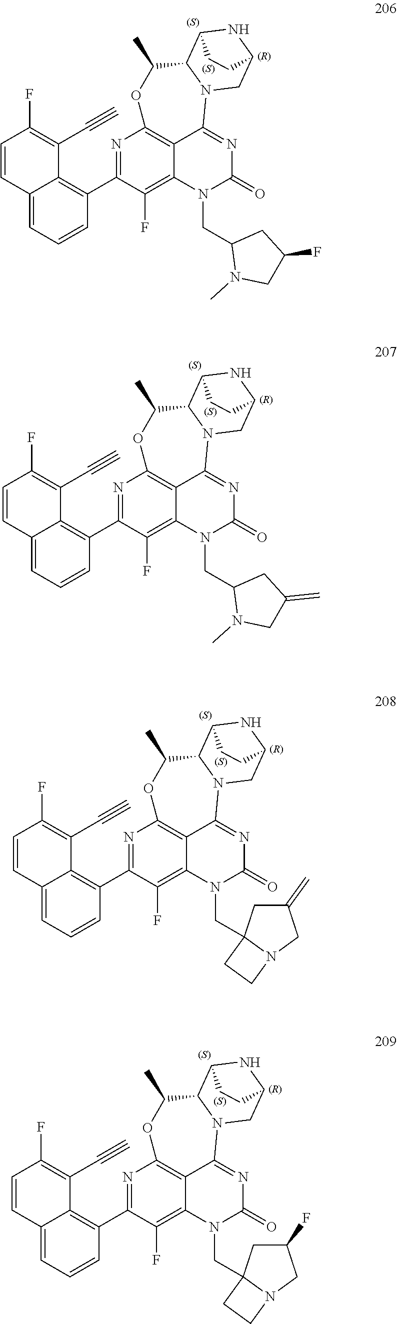

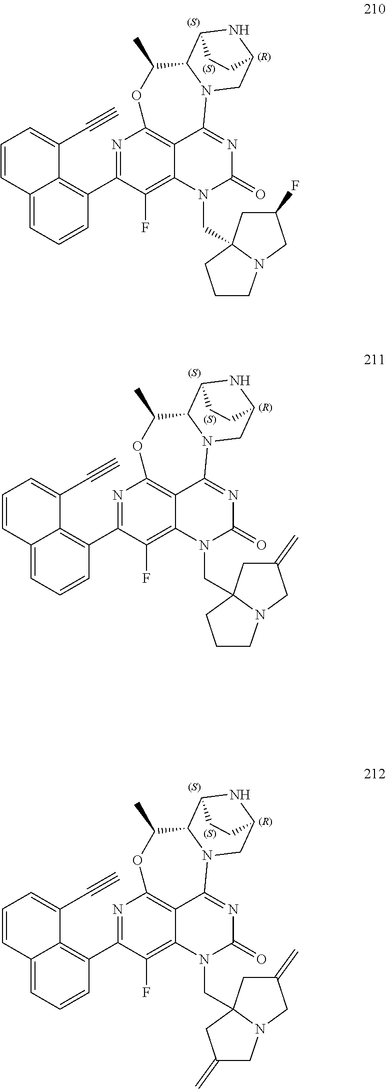

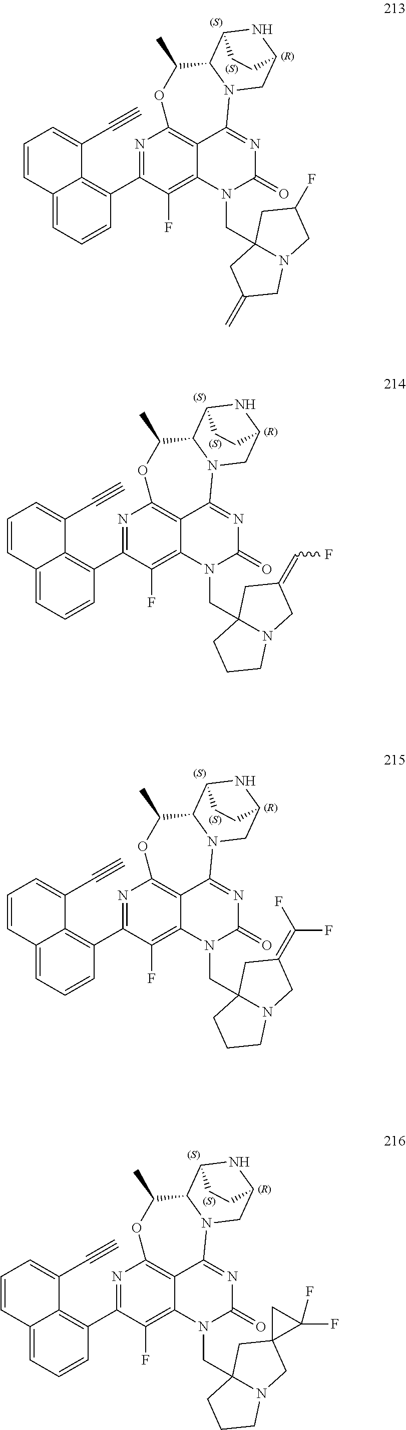

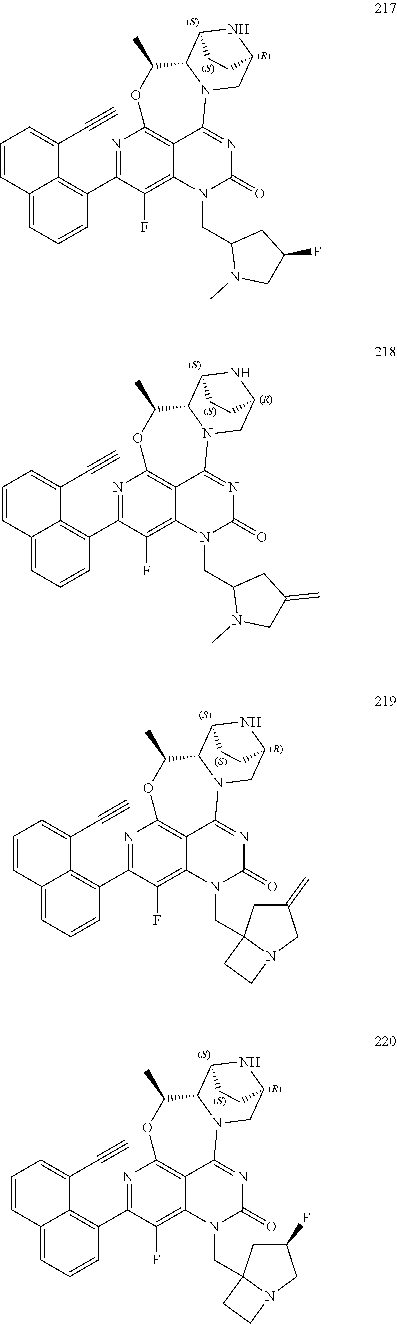

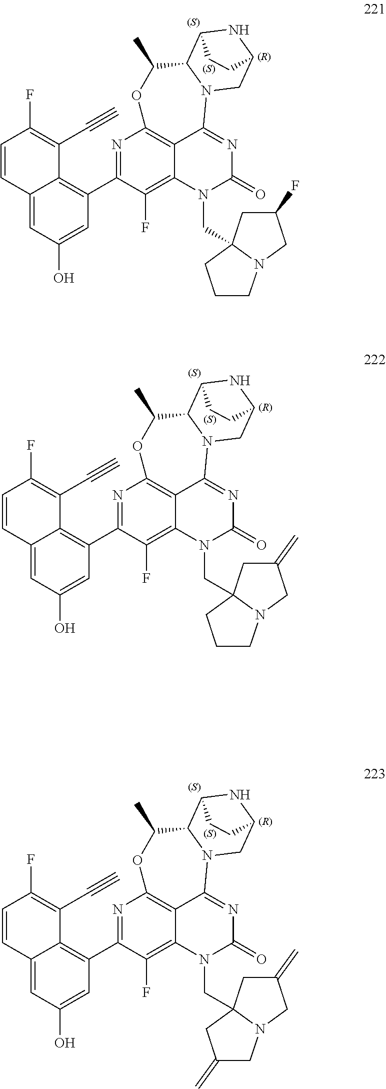

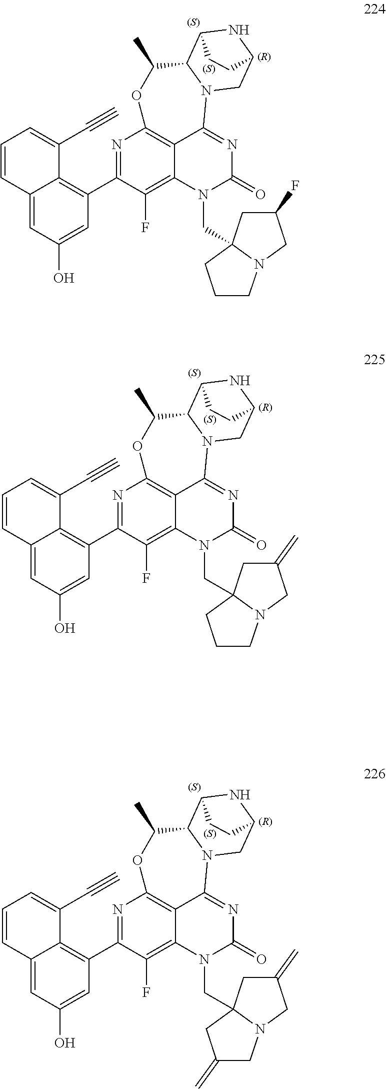

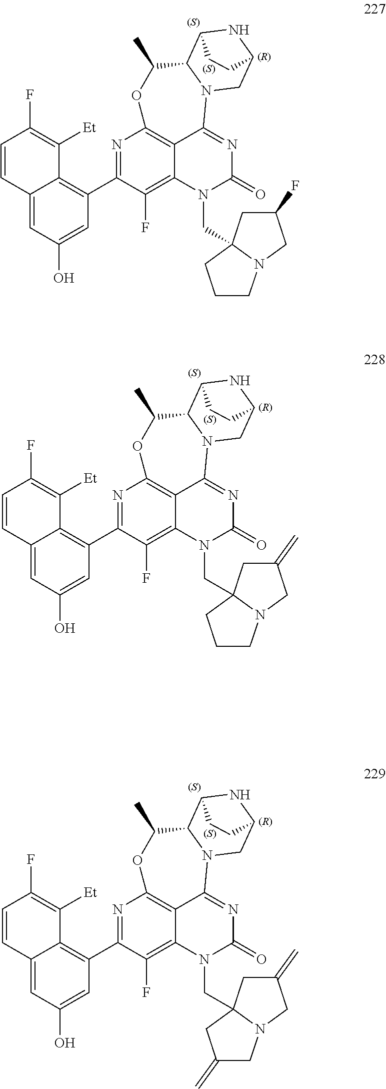

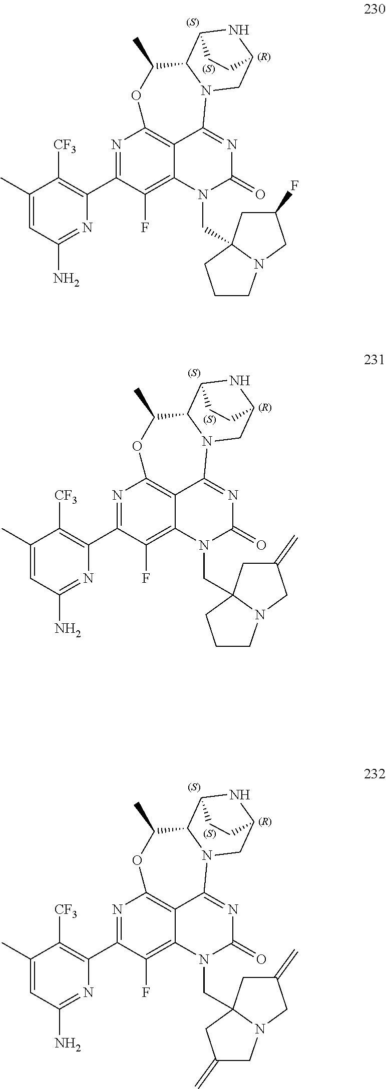

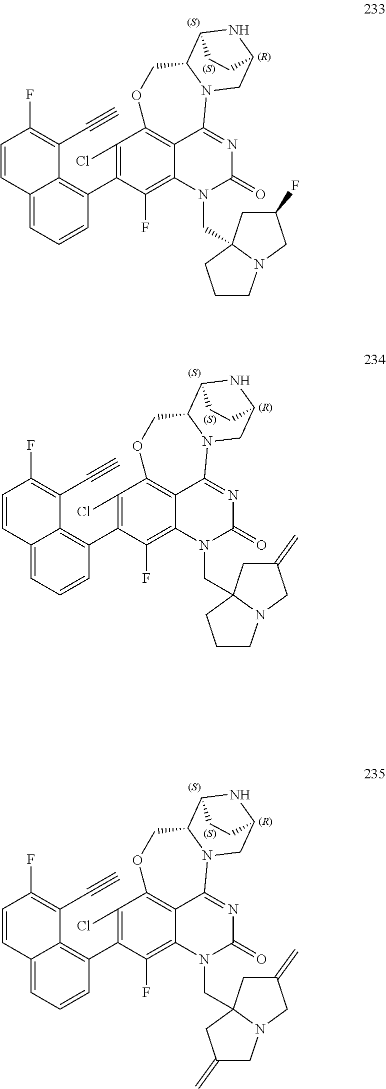

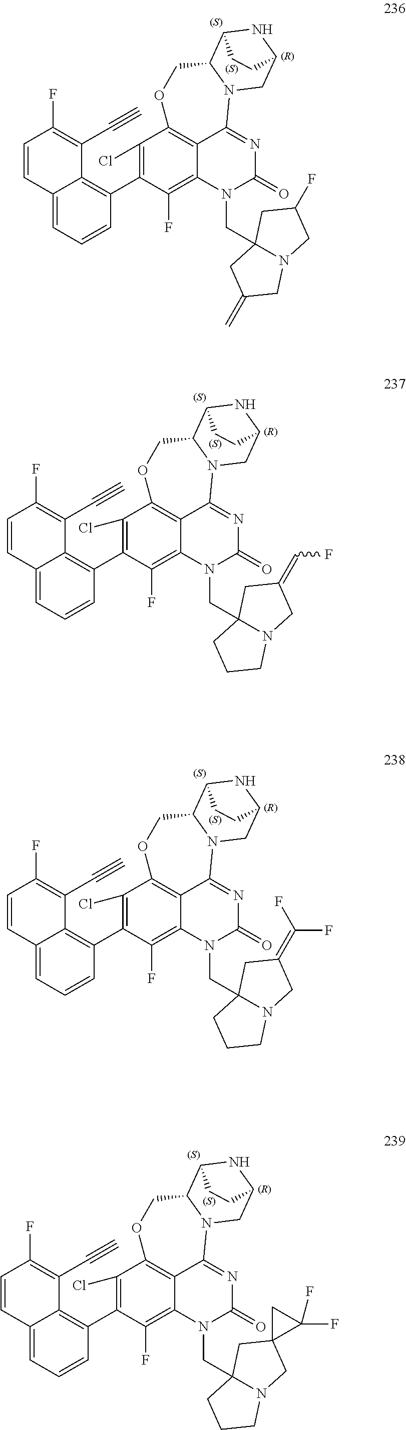

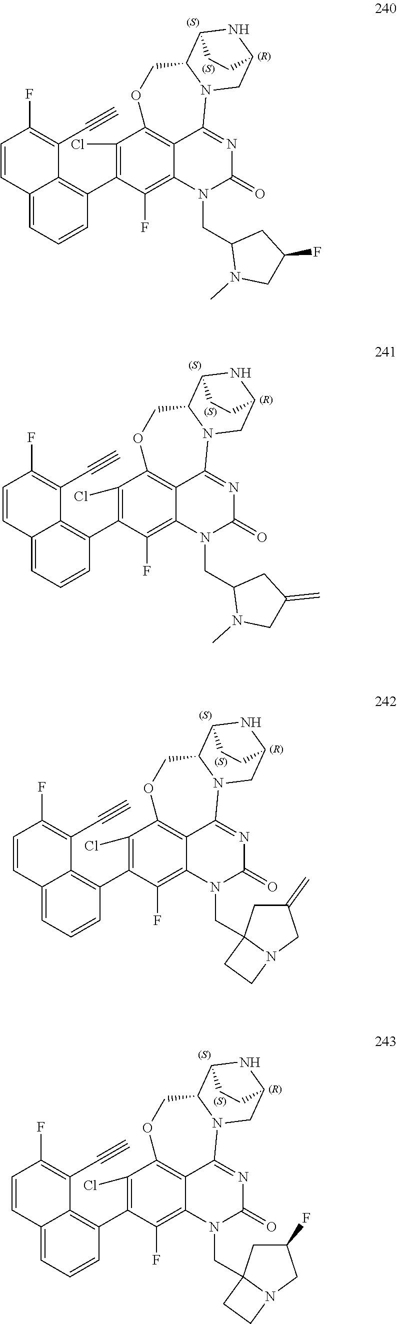

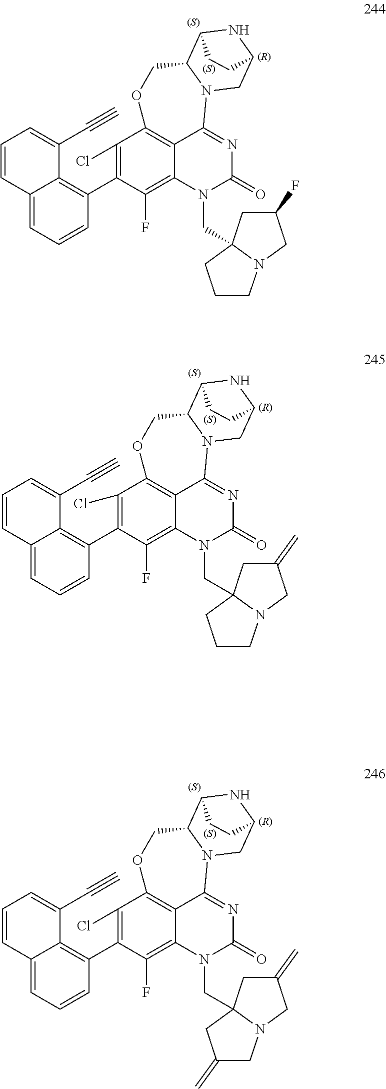

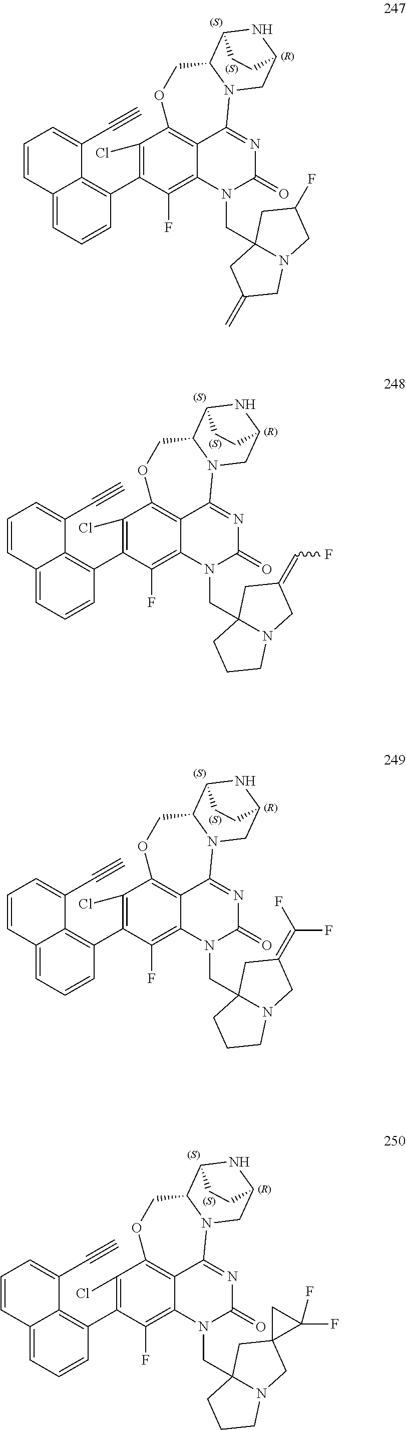

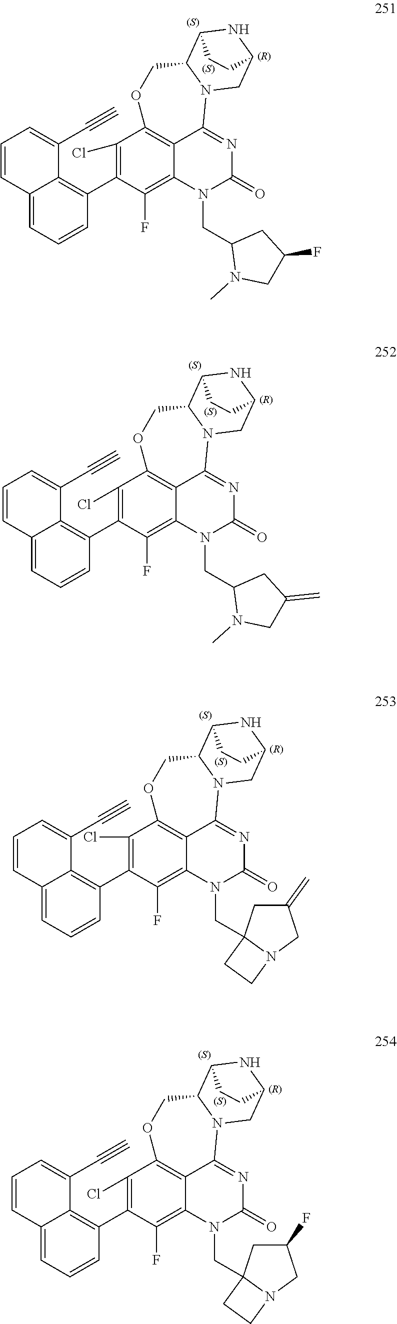

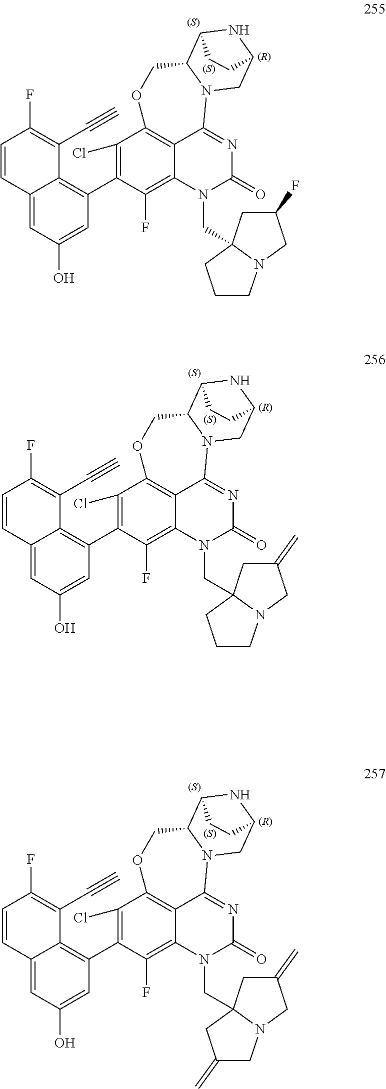

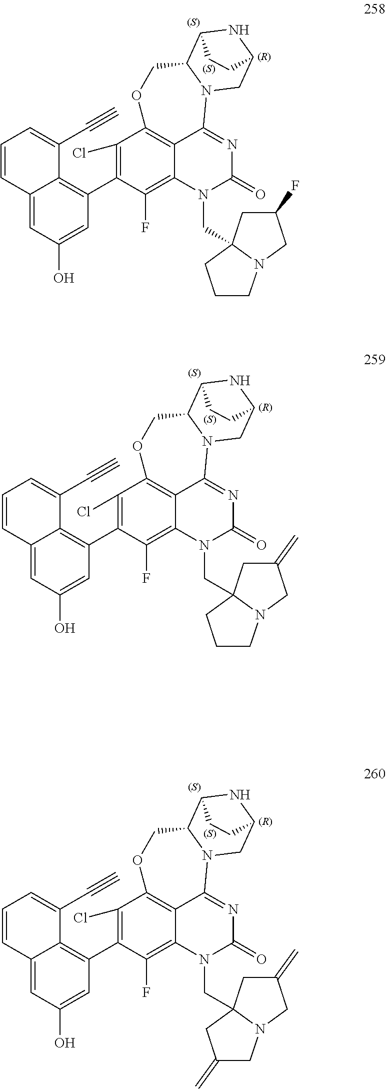

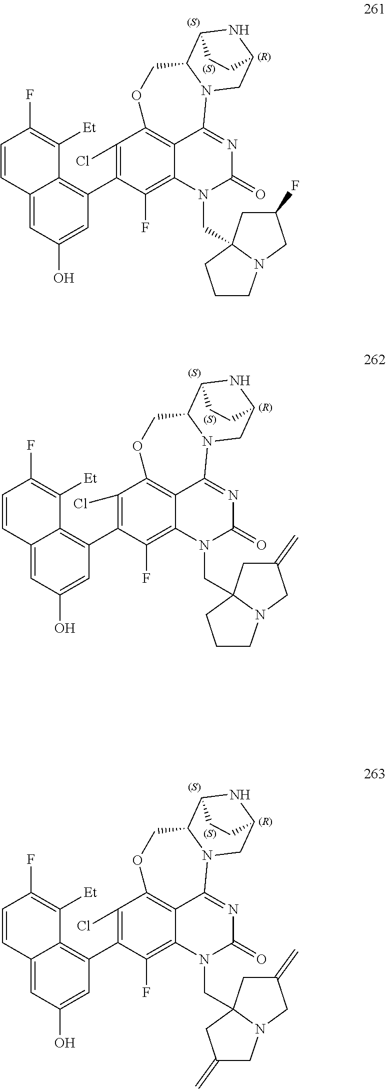

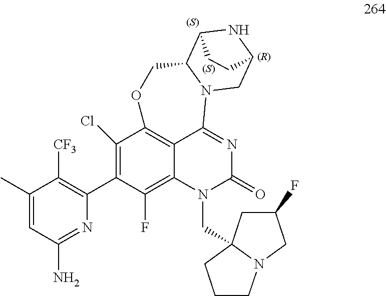

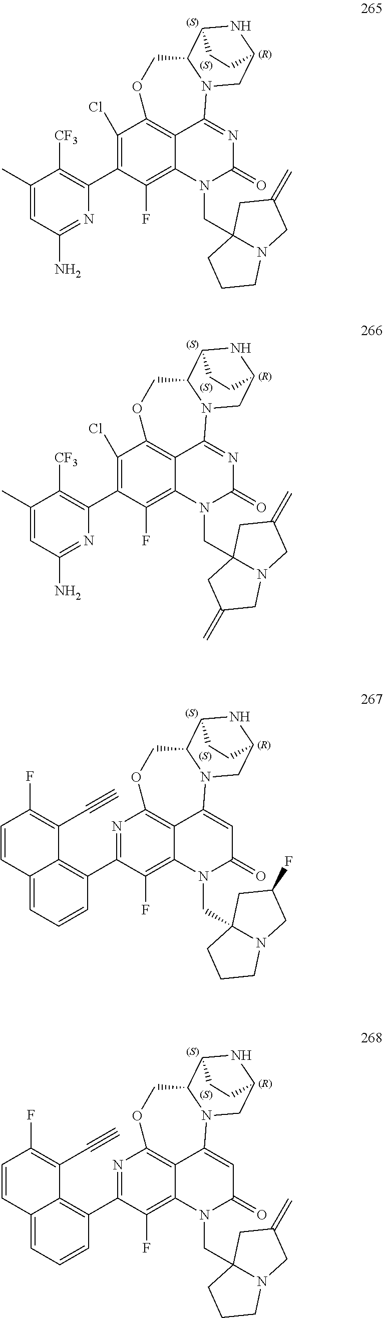

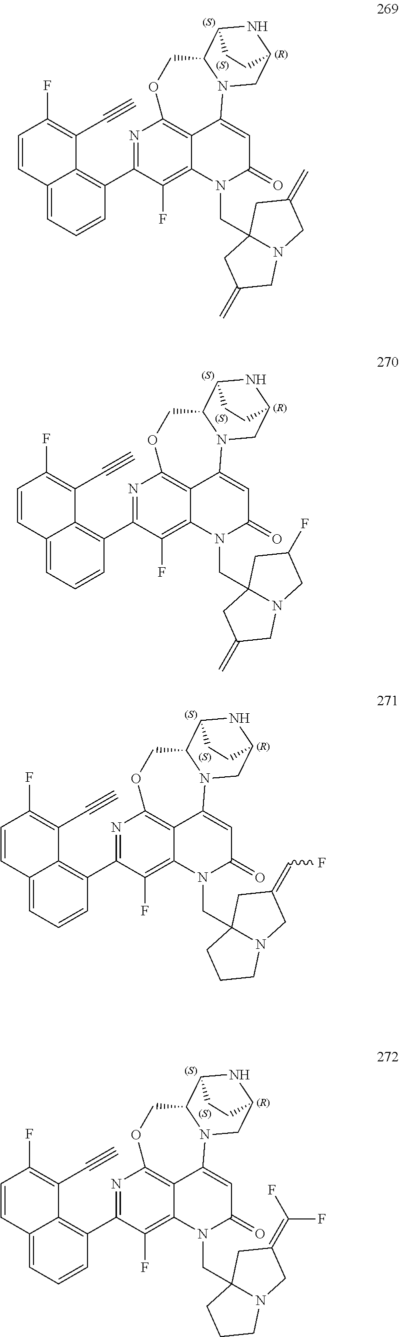

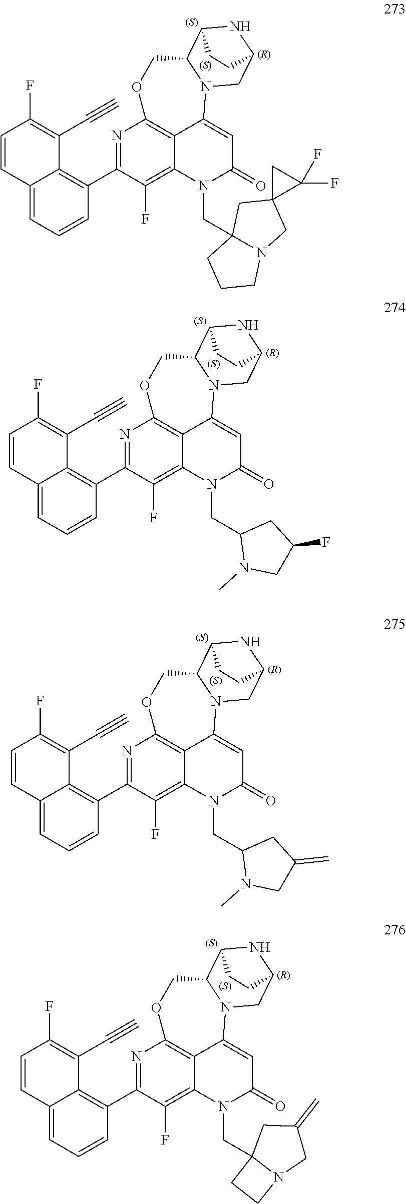

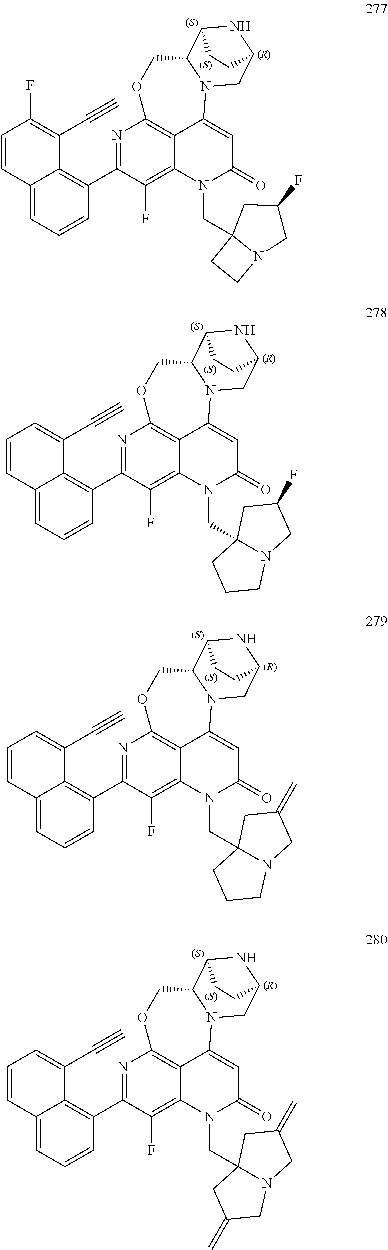

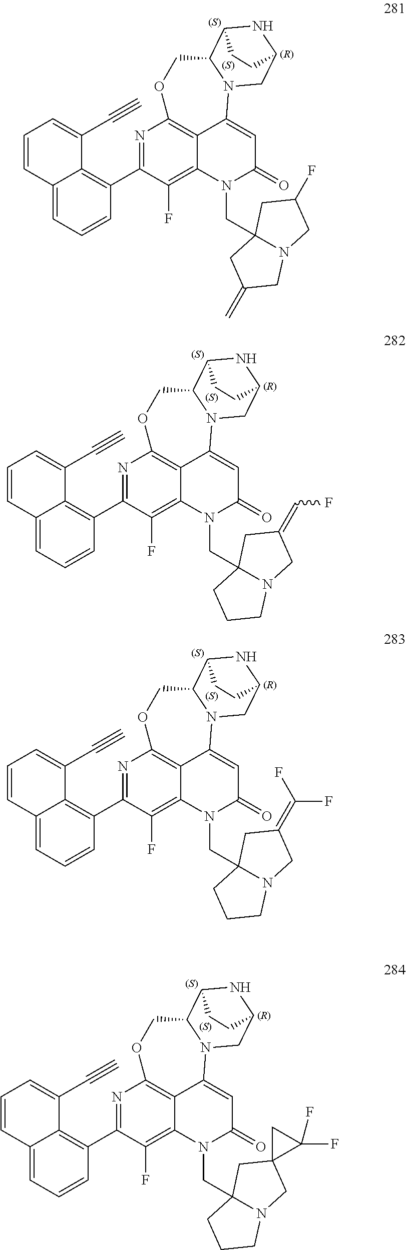

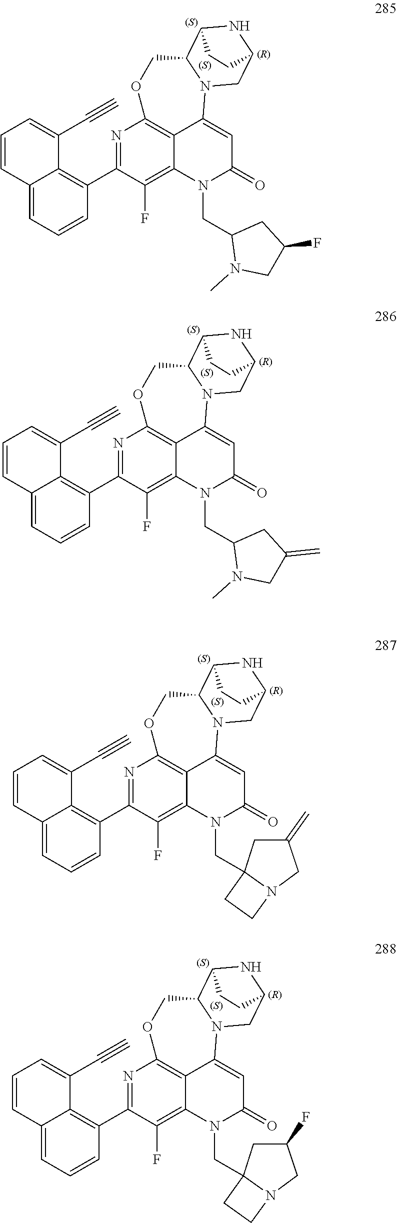

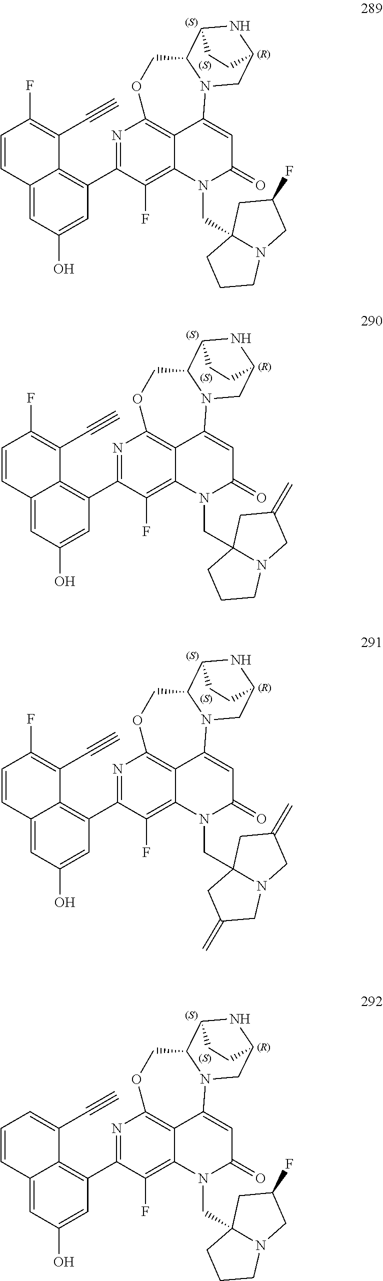

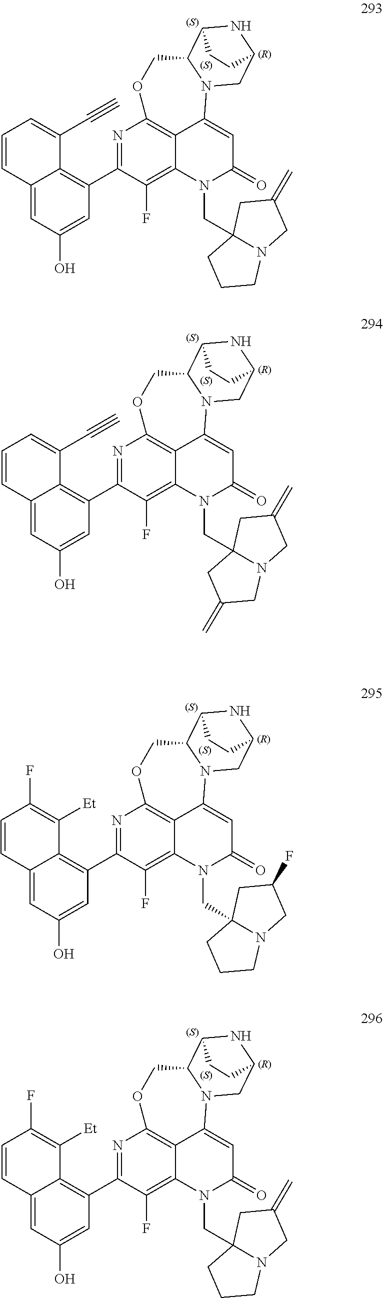

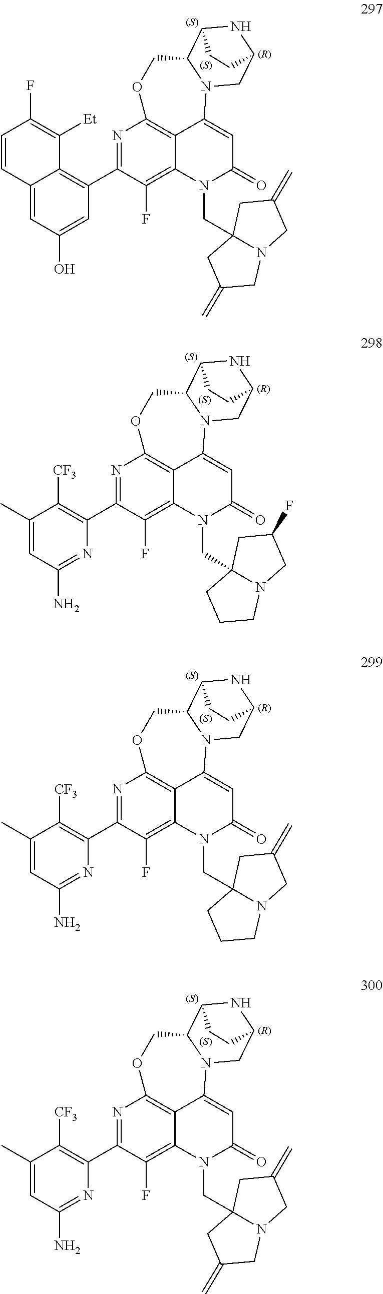

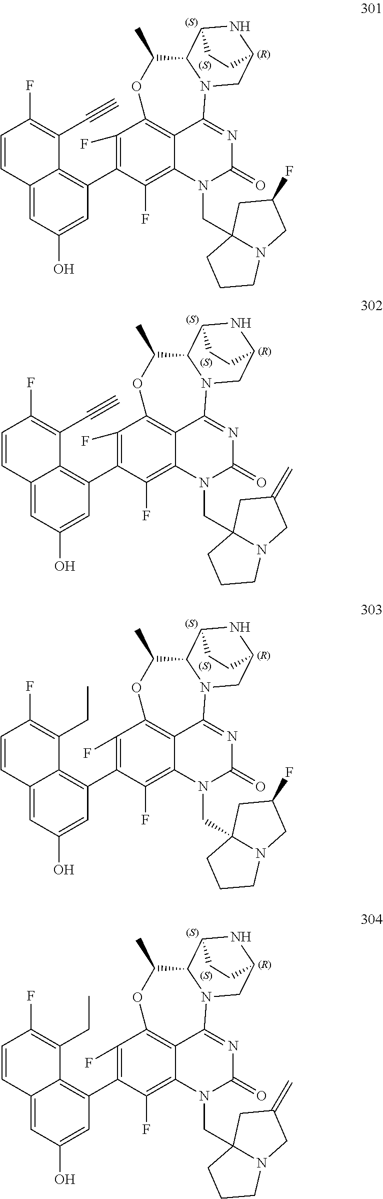

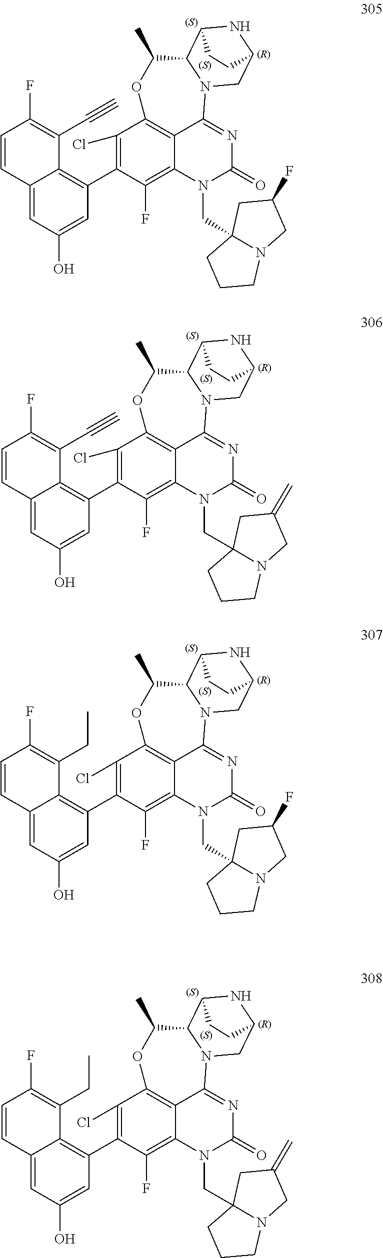

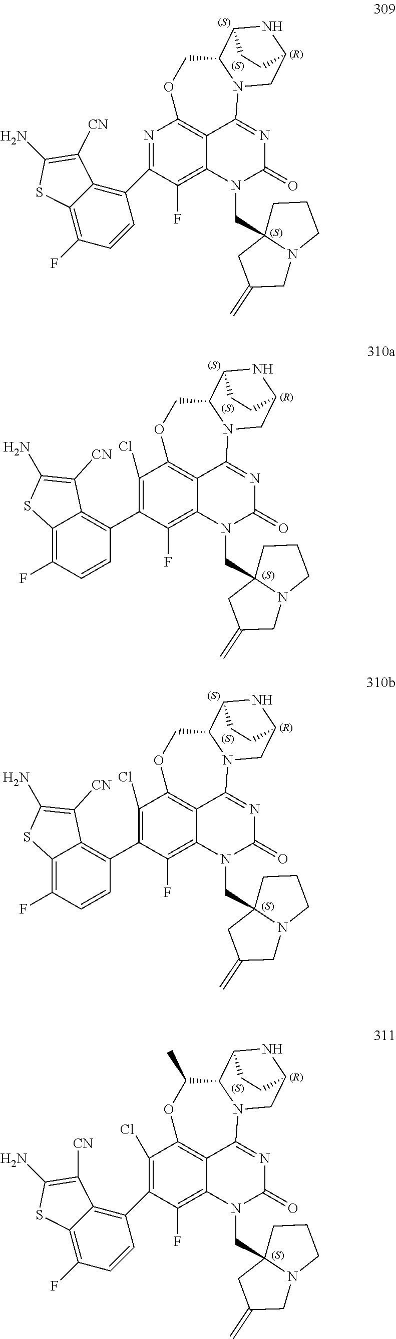

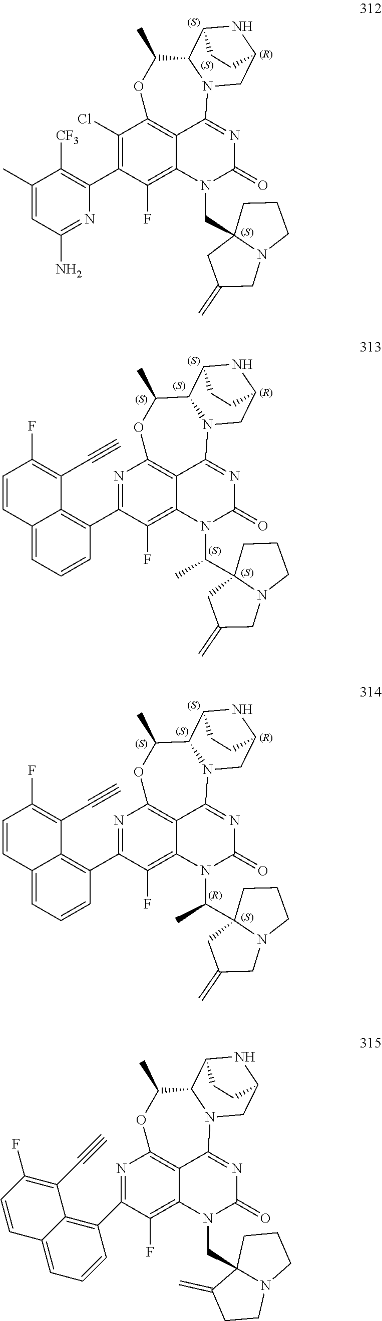

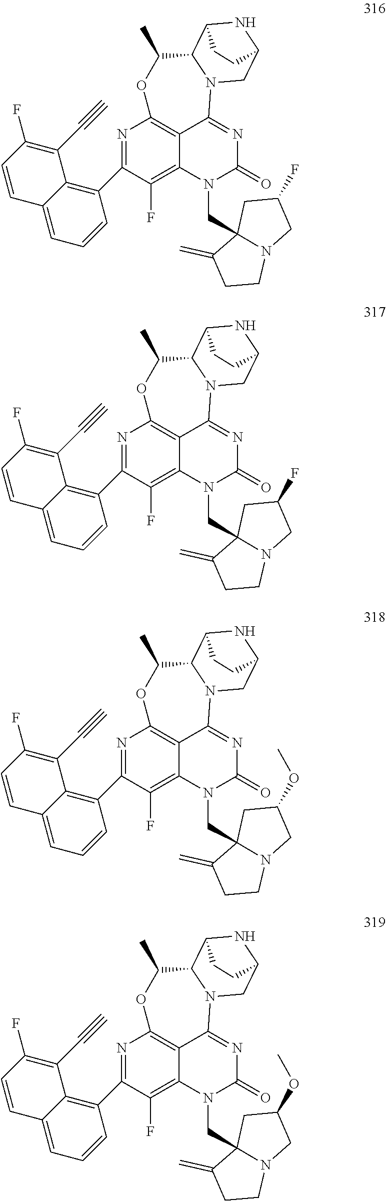

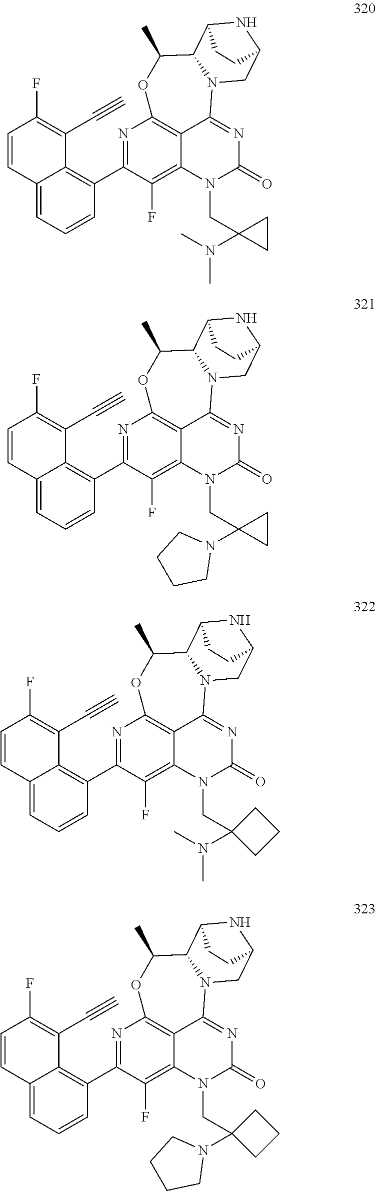

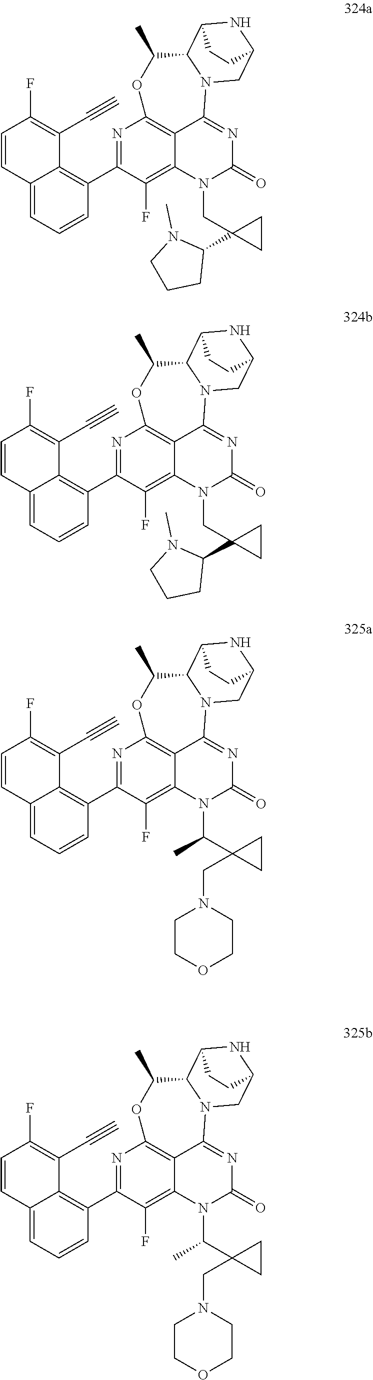 [SMILES: C#Cc1c(F)ccc2cccc(-c3nc4c5c(nc(=O)n(CC6([C@@H]7CCCN7C)CC6)c5c3F)N3C[C@H]5CC[C@H](N5)[C@H]3[C@H](C)O4)c12 ; C#Cc1c(F)ccc2cccc(-c3nc4c5c(nc(=O)n(CC6([C@H]7CCCN7C)CC6)c5c3F)N3C[C@H]5CC[C@H](N5)[C@H]3[C@H](C)O4)c12 ; C#Cc1c(F)ccc2cccc(-c3nc4c5c(nc(=O)n([C@H](C)C6(CN7CCOCC7)CC6)c5c3F)N3C[C@H]5CC[C@H](N5)[C@H]3[C@H](C)O4)c12 ; C#Cc1c(F)ccc2cccc(-c3nc4c5c(nc(=O)n([C@@H](C)C6(CN7CCOCC7)CC6)c5c3F)N3C[C@H]5CC[C@H](N5)[C@H]3[C@H](C)O4)c12]

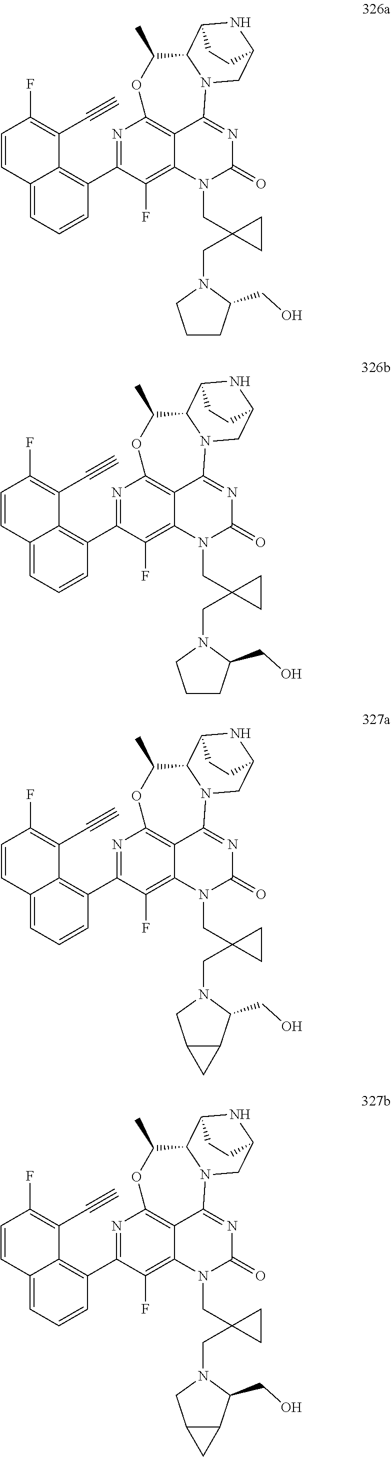

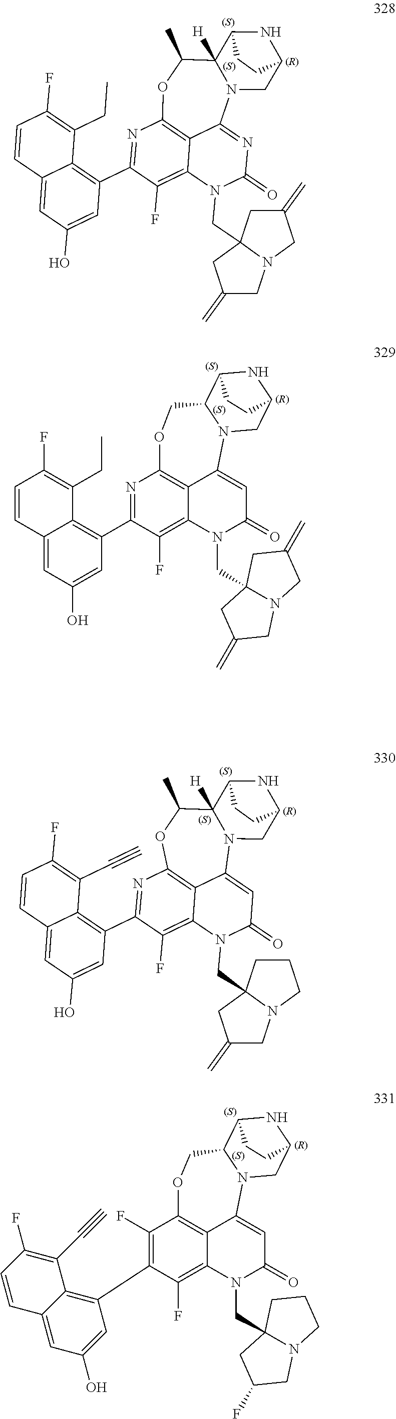

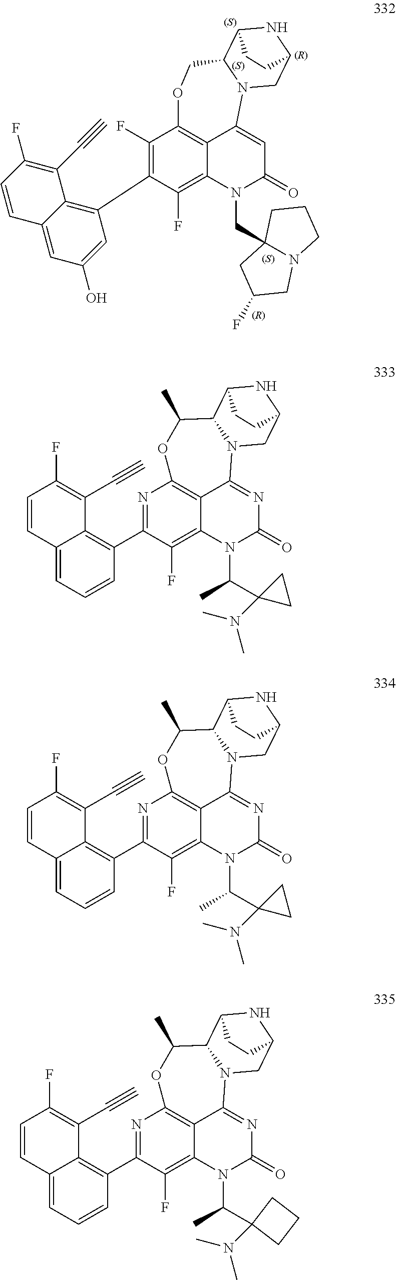

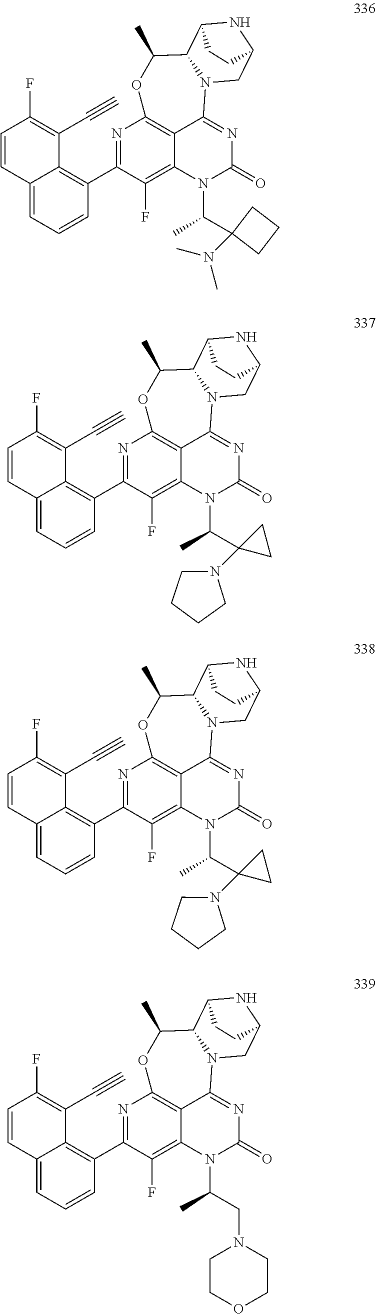

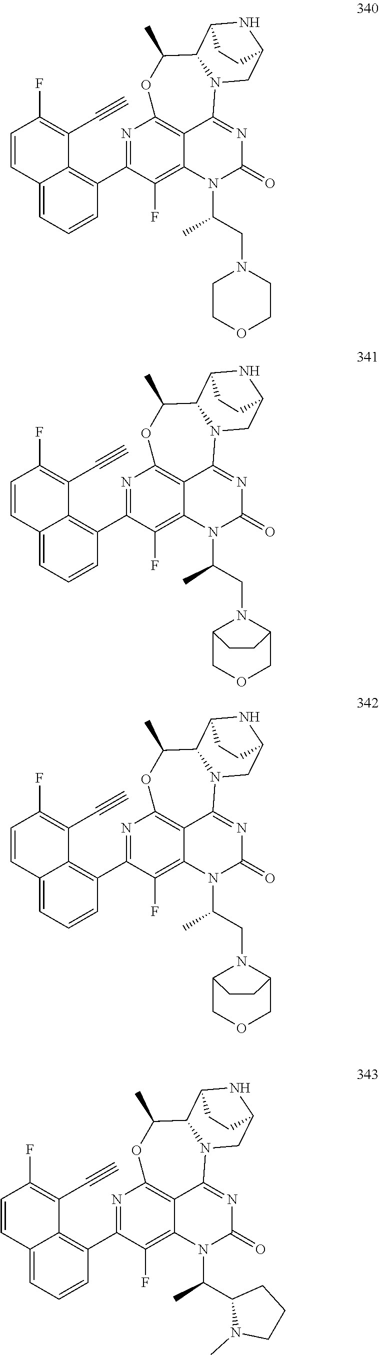

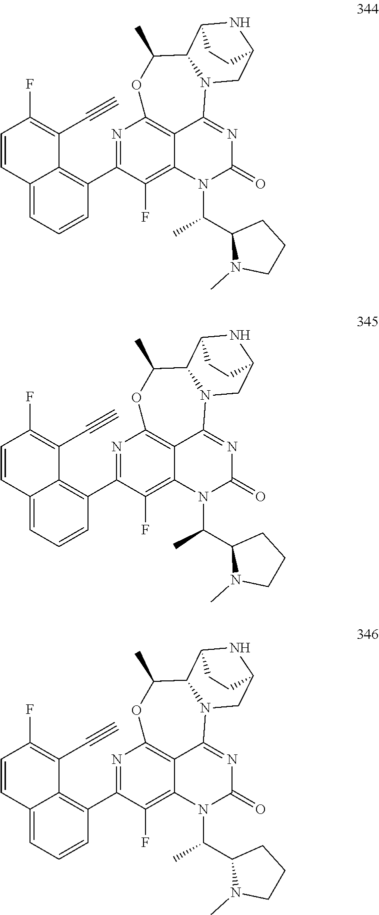 [SMILES: C#Cc1c(F)ccc2cccc(-c3nc4c5c(nc(=O)n([C@@H](C)[C@H]6CCCN6C)c5c3F)N3C[C@H]5CC[C@H](N5)[C@H]3[C@H](C)O4)c12 ; C#Cc1c(F)ccc2cccc(-c3nc4c5c(nc(=O)n([C@H](C)[C@H]6CCCN6C)c5c3F)N3C[C@H]5CC[C@H](N5)[C@H]3[C@H](C)O4)c12 ; C#Cc1c(F)ccc2cccc(-c3nc4c5c(nc(=O)n([C@@H](C)[C@@H]6CCCN6C)c5c3F)N3C[C@H]5CC[C@H](N5)[C@H]3[C@H](C)O4)c12]

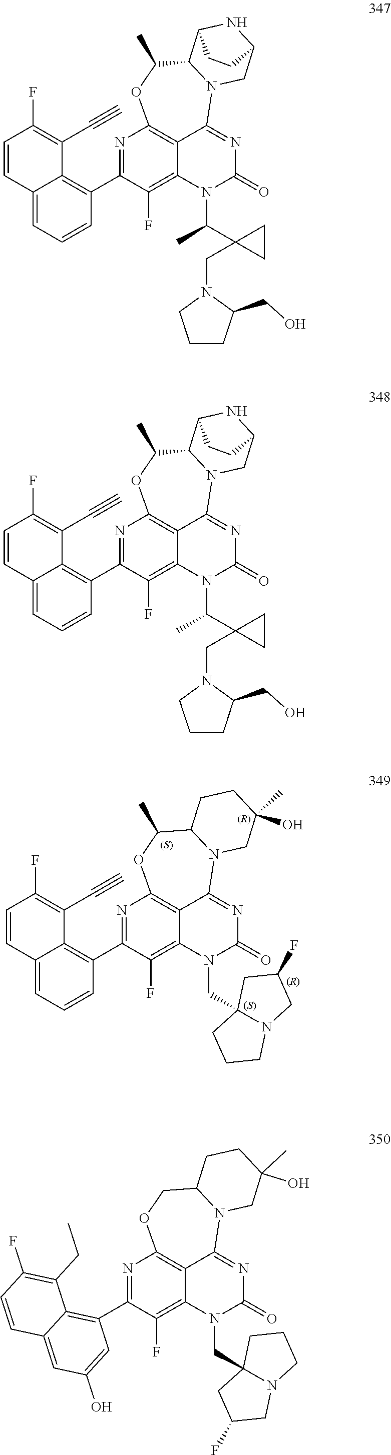

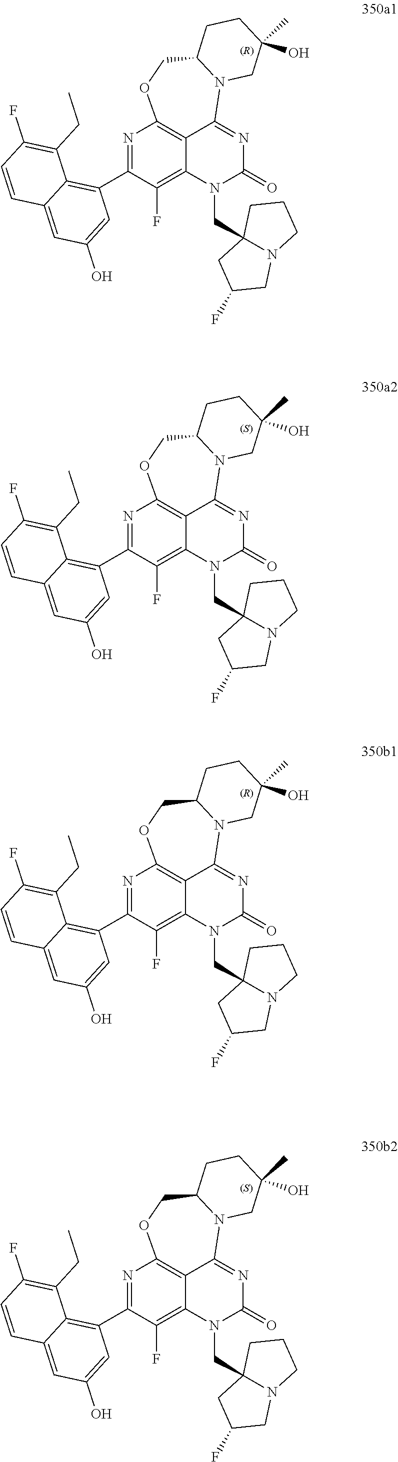

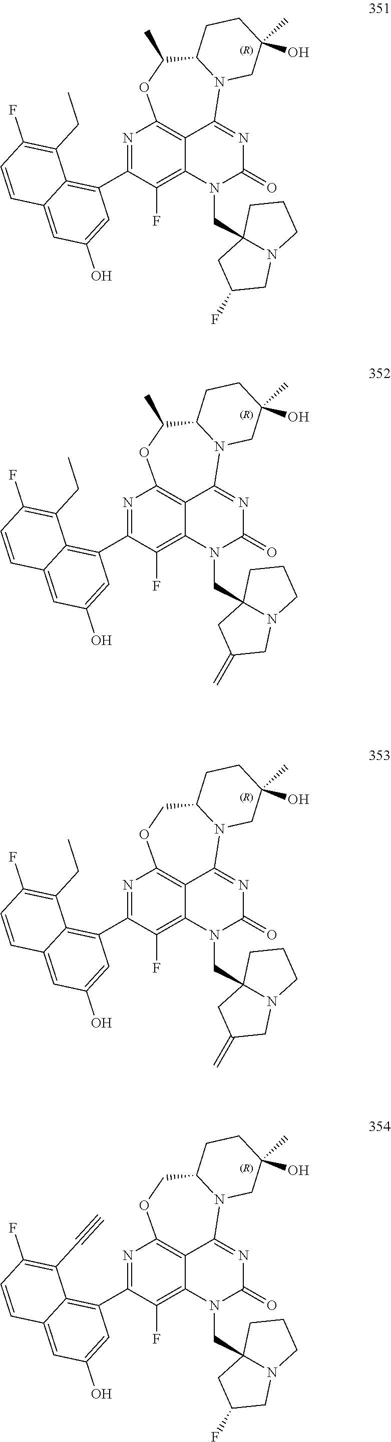

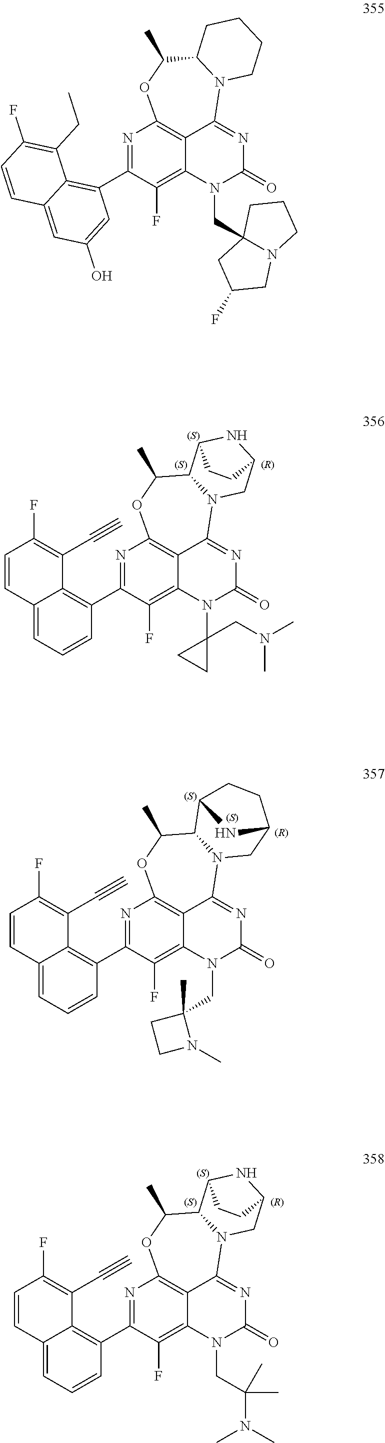

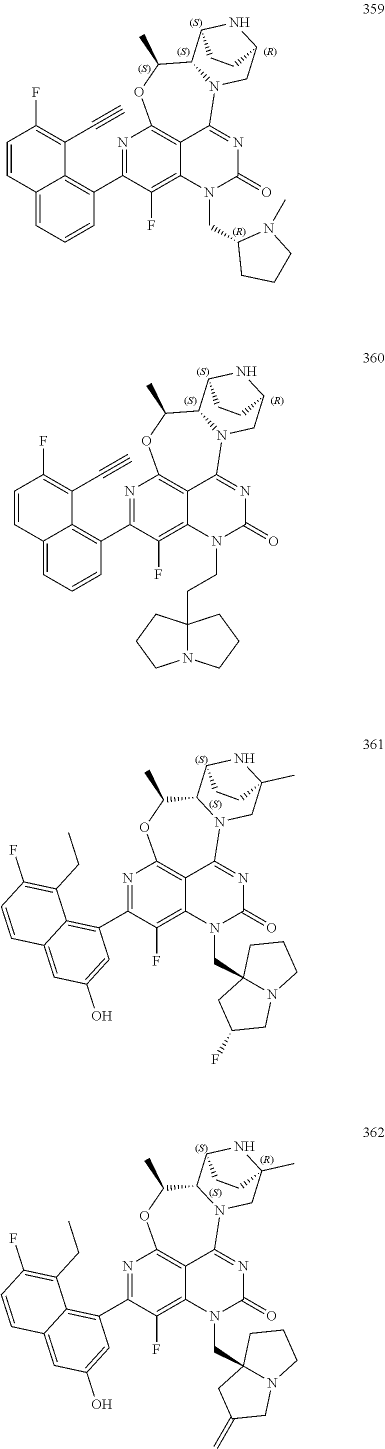

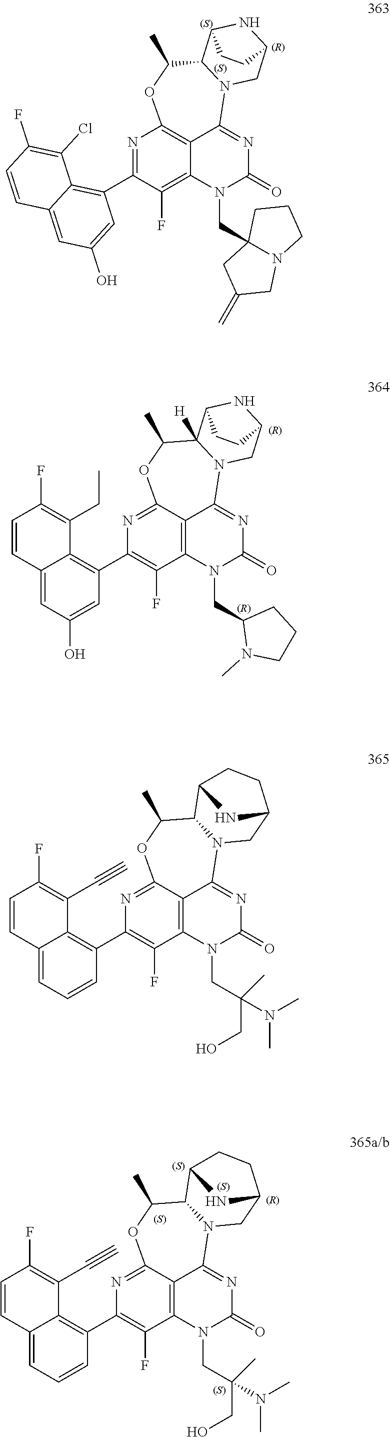

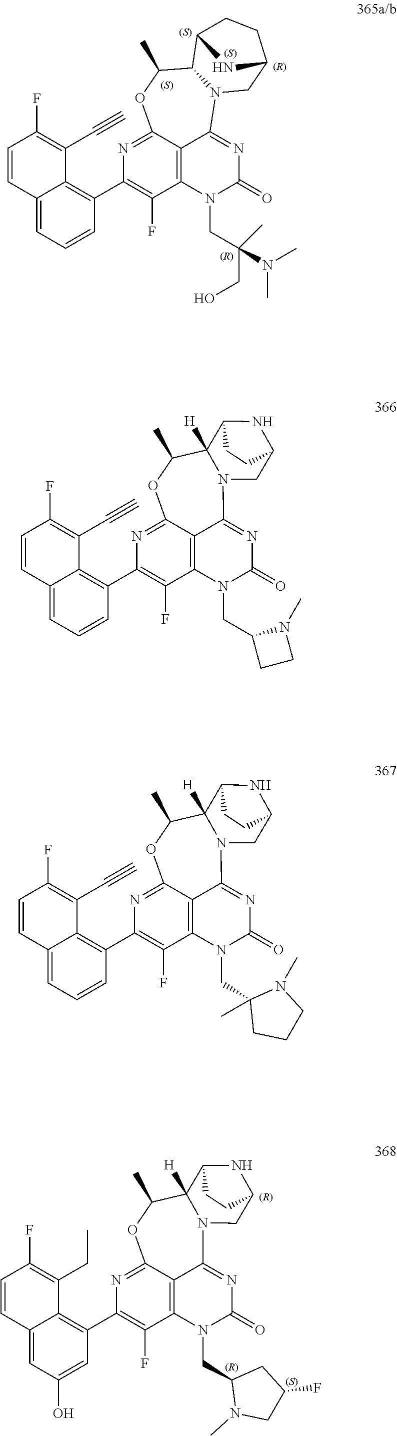

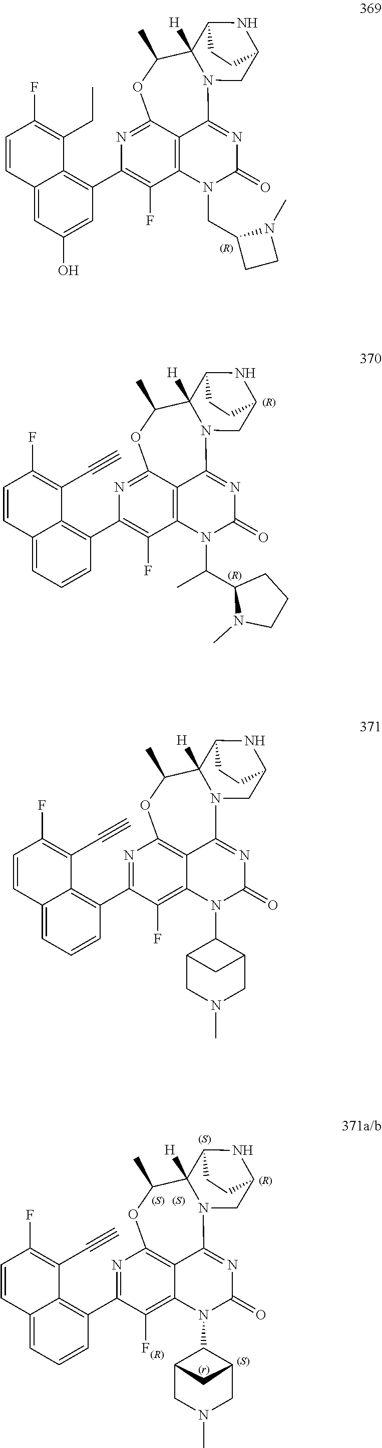

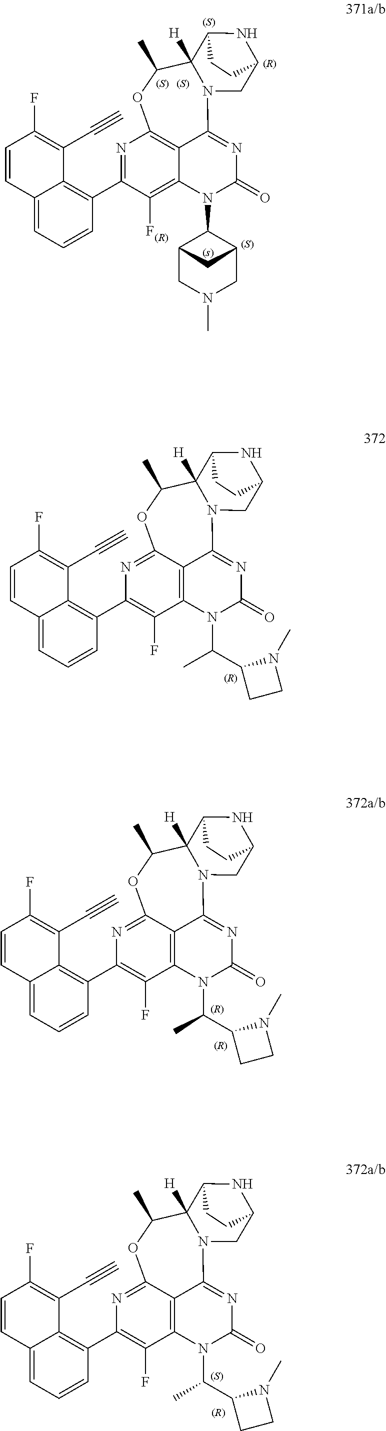

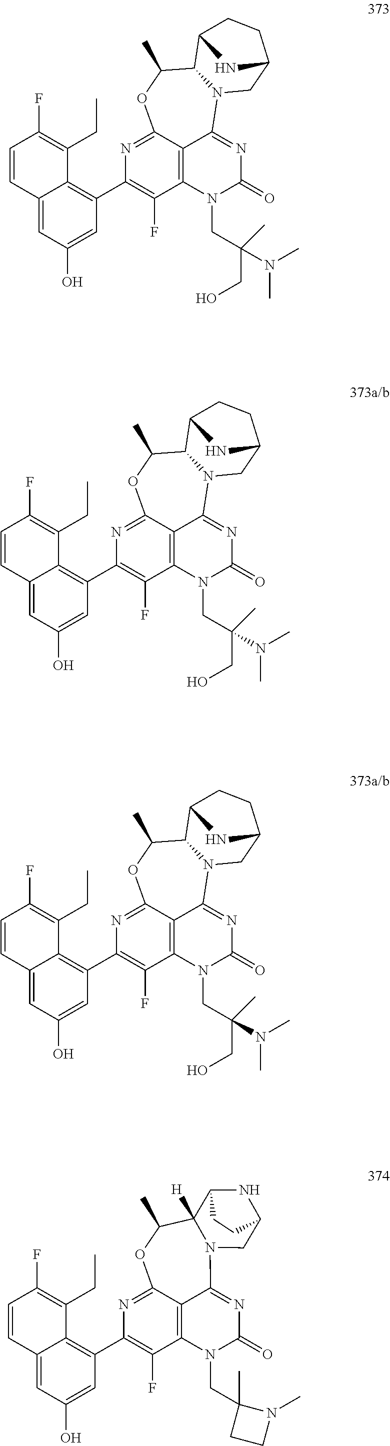 [SMILES: CCc1c(F)ccc2cc(O)cc(-c3nc4c5c(nc(=O)n(CC(C)(CO)N(C)C)c5c3F)N3C[C@H]5CC[C@H](N5)[C@H]3[C@H](C)O4)c12 ; CCc1c(F)ccc2cc(O)cc(-c3nc4c5c(nc(=O)n(C[C@@](C)(CO)N(C)C)c5c3F)N3C[C@H]5CC[C@H](N5)[C@H]3[C@H](C)O4)c12 ; CCc1c(F)ccc2cc(O)cc(-c3nc4c5c(nc(=O)n(C[C@](C)(CO)N(C)C)c5c3F)N3C[C@H]5CC[C@H](N5)[C@H]3[C@H](C)O4)c12 ; CCc1c(F)ccc2cc(O)cc(-c3nc4c5c(nc(=O)n(CC6(C)CCN6C)c5c3F)N3C[C@H]5CC[C@H](N5)[C@H]3[C@H](C)O4)c12]

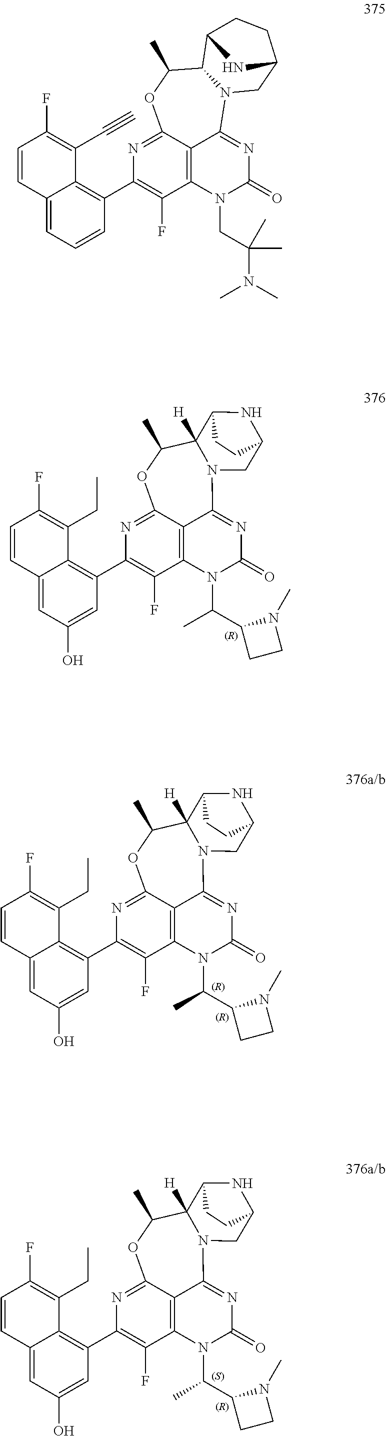

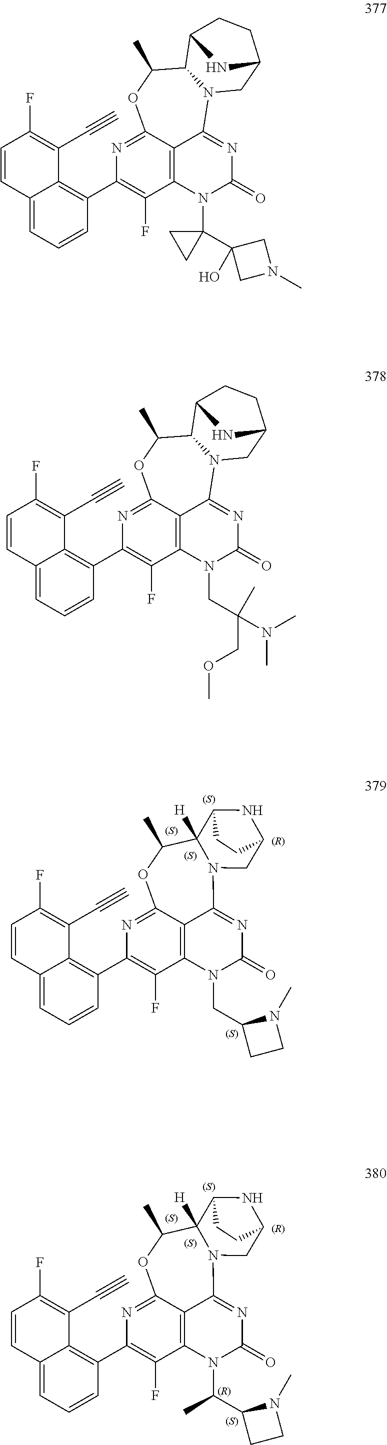

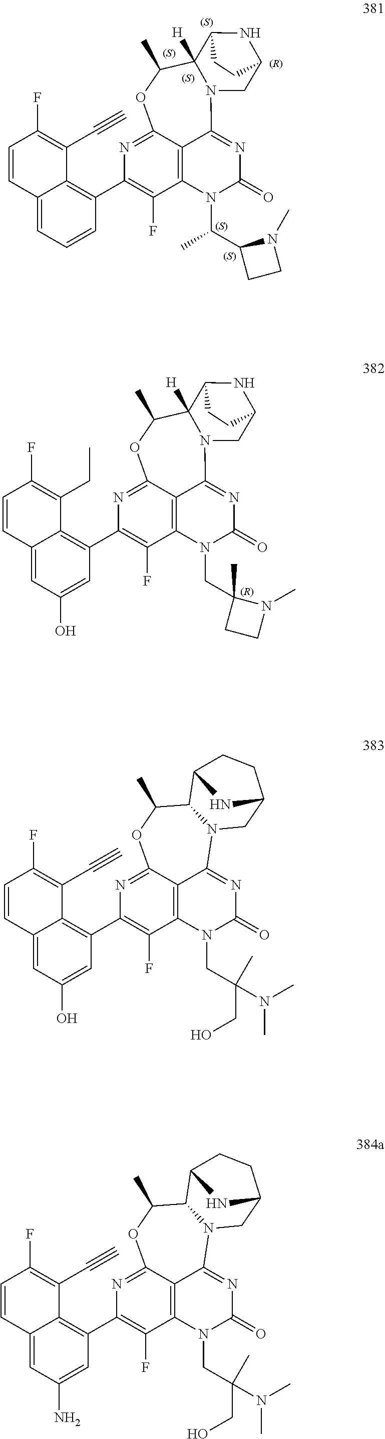

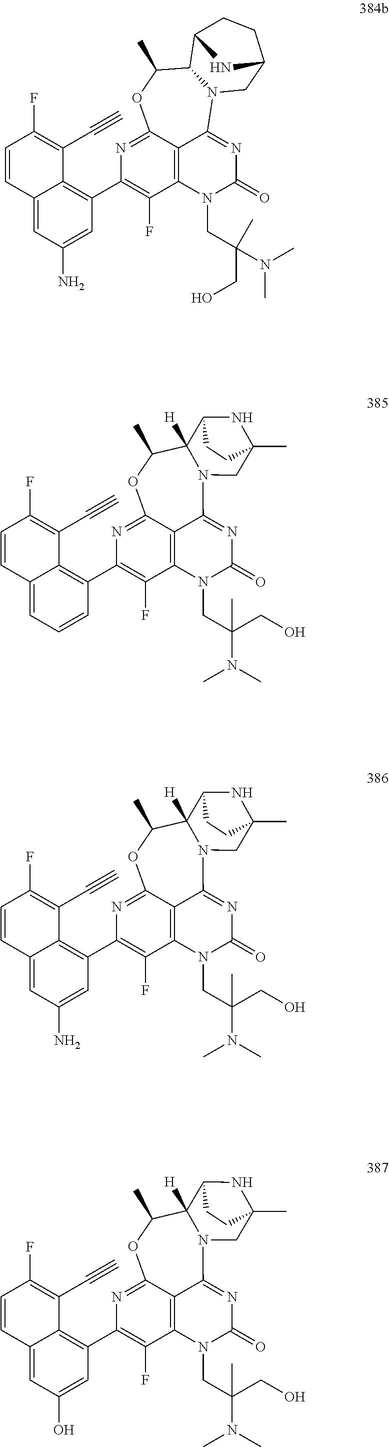 [SMILES: C#Cc1c(F)ccc2cc(N)cc(-c3nc4c5c(nc(=O)n(CC(C)(CO)N(C)C)c5c3F)N3C[C@H]5CC[C@H](N5)[C@H]3[C@H](C)O4)c12 ; C#Cc1c(F)ccc2cccc(-c3nc4c5c(nc(=O)n(CC(C)(CO)N(C)C)c5c3F)N3C[C@@]5(C)CC[C@H](N5)[C@H]3[C@H](C)O4)c12 ; C#Cc1c(F)ccc2cc(N)cc(-c3nc4c5c(nc(=O)n(CC(C)(CO)N(C)C)c5c3F)N3C[C@@]5(C)CC[C@H](N5)[C@H]3[C@H](C)O4)c12 ; C#Cc1c(F)ccc2cc(O)cc(-c3nc4c5c(nc(=O)n(CC(C)(CO)N(C)C)c5c3F)N3C[C@@]5(C)CC[C@H](N5)[C@H]3[C@H](C)O4)c12]

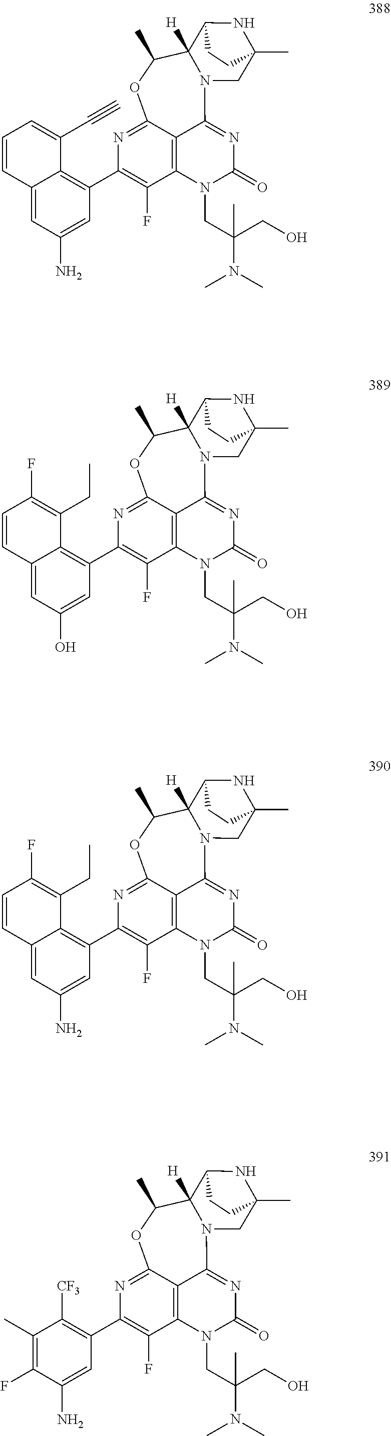 [SMILES: C#Cc1cccc2cc(N)cc(-c3nc4c5c(nc(=O)n(CC(C)(CO)N(C)C)c5c3F)N3C[C@@]5(C)CC[C@H](N5)[C@H]3[C@H](C)O4)c12 ; CCc1c(F)ccc2cc(O)cc(-c3nc4c5c(nc(=O)n(CC(C)(CO)N(C)C)c5c3F)N3C[C@@]5(C)CC[C@H](N5)[C@H]3[C@H](C)O4)c12 ; CCc1c(F)ccc2cc(N)cc(-c3nc4c5c(nc(=O)n(CC(C)(CO)N(C)C)c5c3F)N3C[C@@]5(C)CC[C@H](N5)[C@H]3[C@H](C)O4)c12 ; Cc1c(F)c(N)cc(-c2nc3c4c(nc(=O)n(CC(C)(CO)N(C)C)c4c2F)N2C[C@@]4(C)CC[C@H](N4)[C@H]2[C@H](C)O3)c1C(F)(F)F]

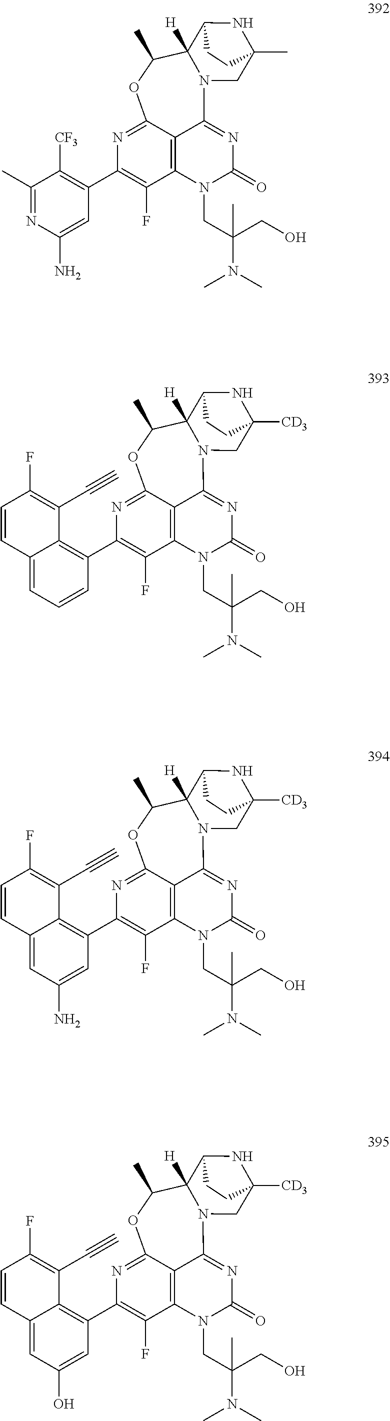 [SMILES: Cc1nc(N)cc(-c2nc3c4c(nc(=O)n(CC(C)(CO)N(C)C)c4c2F)N2C[C@@]4(C)CC[C@H](N4)[C@H]2[C@H](C)O3)c1C(F)(F)F ; [2H]C([2H])([2H])[C@]12CC[C@H](N1)[C@H]1[C@H](C)Oc3nc(-c4cccc5ccc(F)c(C#C)c45)c(F)c4c3c(nc(=O)n4CC(C)(CO)N(C)C)N1C2 ; [2H]C([2H])([2H])[C@]12CC[C@H](N1)[C@H]1[C@H](C)Oc3nc(-c4cc(N)cc5ccc(F)c(C#C)c45)c(F)c4c3c(nc(=O)n4CC(C)(CO)N(C)C)N1C2 ; [2H]C([2H])([2H])[C@]12CC[C@H](N1)[C@H]1[C@H](C)Oc3nc(-c4cc(O)cc5ccc(F)c(C#C)c45)c(F)c4c3c(nc(=O)n4CC(C)(CO)N(C)C)N1C2]

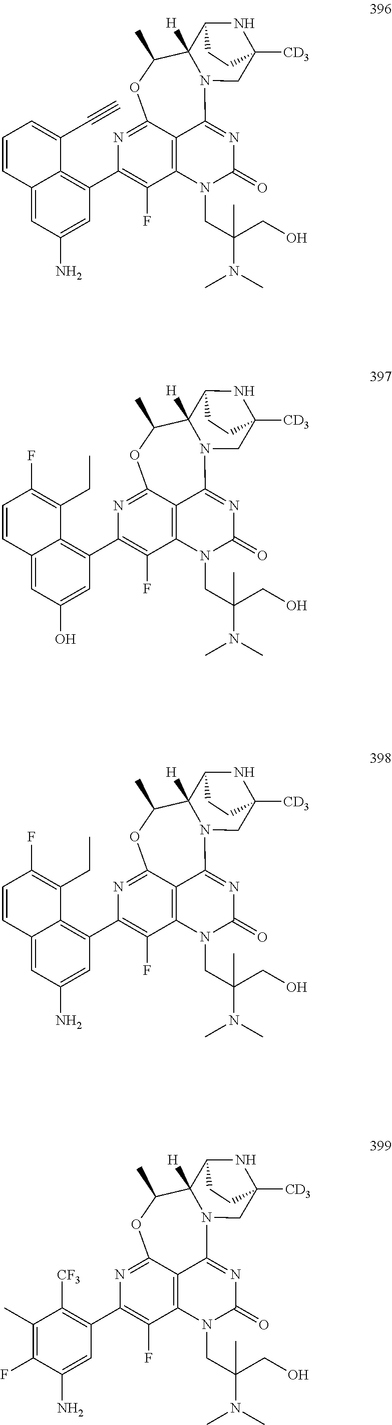 [SMILES: [2H]C([2H])([2H])[C@]12CC[C@H](N1)[C@H]1[C@H](C)Oc3nc(-c4cc(N)cc5cccc(C#C)c45)c(F)c4c3c(nc(=O)n4CC(C)(CO)N(C)C)N1C2 ; [2H]C([2H])([2H])[C@]12CC[C@H](N1)[C@H]1[C@H](C)Oc3nc(-c4cc(O)cc5ccc(F)c(CC)c45)c(F)c4c3c(nc(=O)n4CC(C)(CO)N(C)C)N1C2 ; [2H]C([2H])([2H])[C@]12CC[C@H](N1)[C@H]1[C@H](C)Oc3nc(-c4cc(N)cc5ccc(F)c(CC)c45)c(F)c4c3c(nc(=O)n4CC(C)(CO)N(C)C)N1C2 ; [2H]C([2H])([2H])[C@]12CC[C@H](N1)[C@H]1[C@H](C)Oc3nc(-c4cc(N)c(F)c(C)c4C(F)(F)F)c(F)c4c3c(nc(=O)n4CC(C)(CO)N(C)C)N1C2]

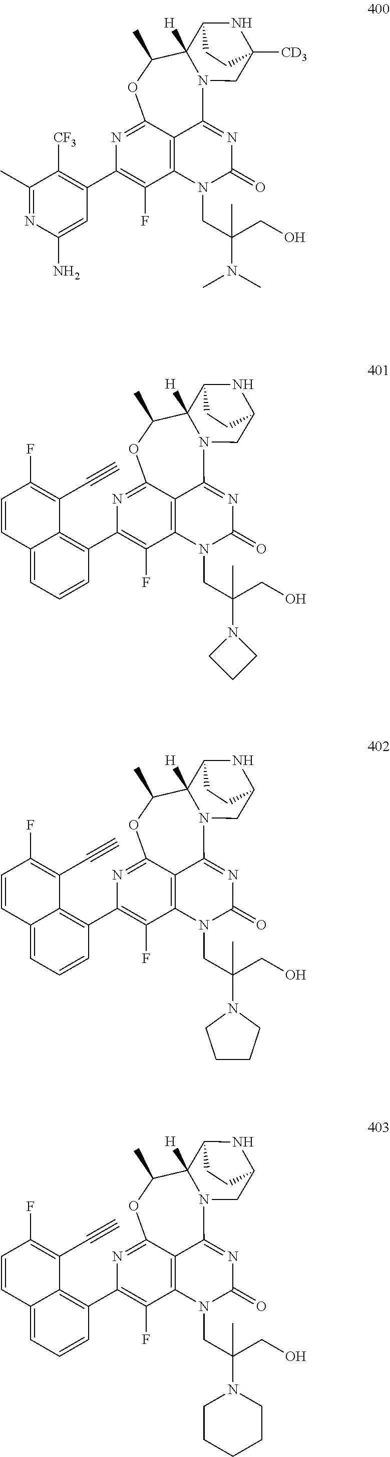 [SMILES: [2H]C([2H])([2H])[C@]12CC[C@H](N1)[C@H]1[C@H](C)Oc3nc(-c4cc(N)nc(C)c4C(F)(F)F)c(F)c4c3c(nc(=O)n4CC(C)(CO)N(C)C)N1C2 ; C#Cc1c(F)ccc2cccc(-c3nc4c5c(nc(=O)n(CC(C)(CO)N6CCC6)c5c3F)N3C[C@H]5CC[C@H](N5)[C@H]3[C@H](C)O4)c12 ; C#Cc1c(F)ccc2cccc(-c3nc4c5c(nc(=O)n(CC(C)(CO)N6CCCC6)c5c3F)N3C[C@H]5CC[C@H](N5)[C@H]3[C@H](C)O4)c12 ; C#Cc1c(F)ccc2cccc(-c3nc4c5c(nc(=O)n(CC(C)(CO)N6CCCCC6)c5c3F)N3C[C@H]5CC[C@H](N5)[C@H]3[C@H](C)O4)c12]

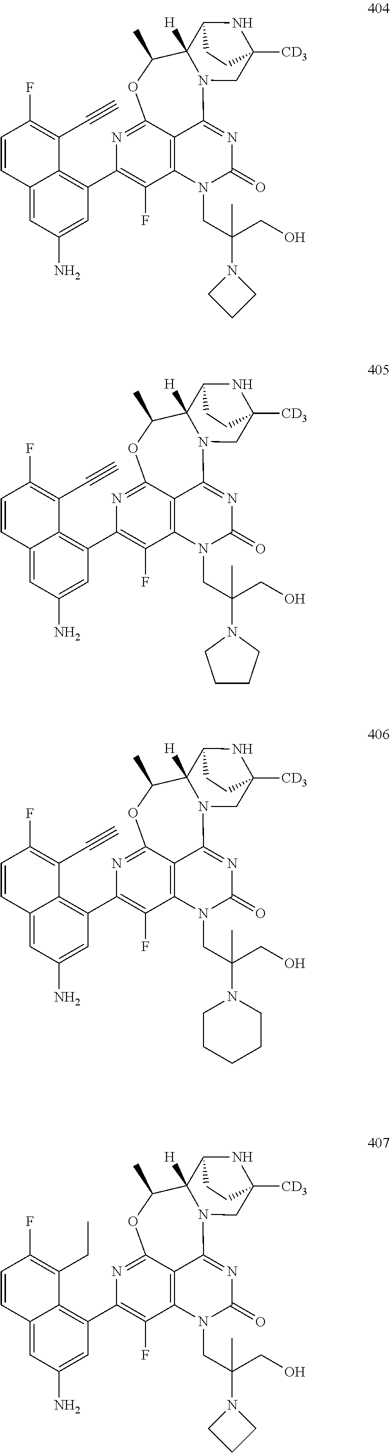 [SMILES: [2H]C([2H])([2H])[C@]12CC[C@H](N1)[C@H]1[C@H](C)Oc3nc(-c4cc(N)cc5ccc(F)c(C#C)c45)c(F)c4c3c(nc(=O)n4CC(C)(CO)N3CCC3)N1C2 ; [2H]C([2H])([2H])[C@]12CC[C@H](N1)[C@H]1[C@H](C)Oc3nc(-c4cc(N)cc5ccc(F)c(C#C)c45)c(F)c4c3c(nc(=O)n4CC(C)(CO)N3CCCC3)N1C2 ; [2H]C([2H])([2H])[C@]12CC[C@H](N1)[C@H]1[C@H](C)Oc3nc(-c4cc(N)cc5ccc(F)c(C#C)c45)c(F)c4c3c(nc(=O)n4CC(C)(CO)N3CCCCC3)N1C2 ; [2H]C([2H])([2H])[C@]12CC[C@H](N1)[C@H]1[C@H](C)Oc3nc(-c4cc(N)cc5ccc(F)c(CC)c45)c(F)c4c3c(nc(=O)n4CC(C)(CO)N3CCC3)N1C2]

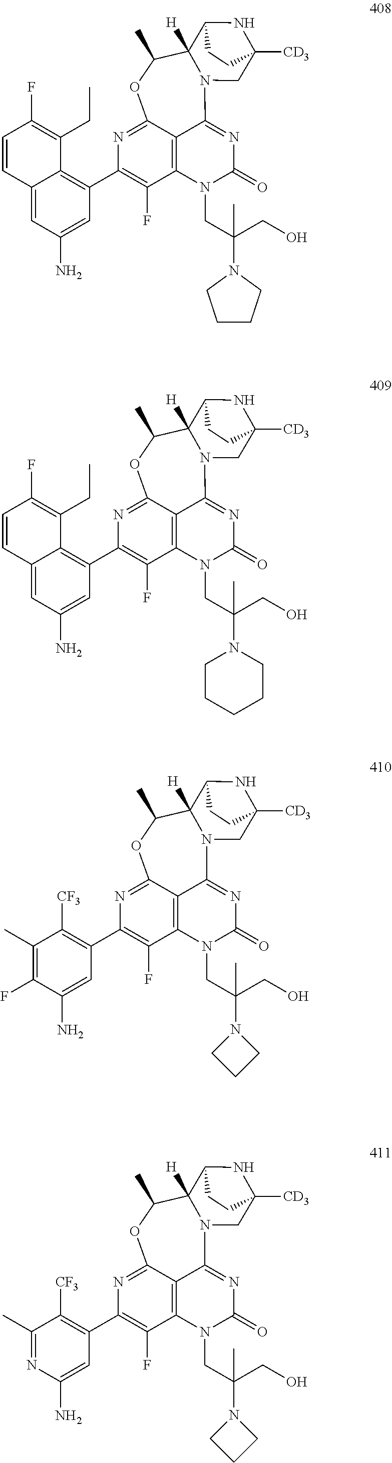 [SMILES: [2H]C([2H])([2H])[C@]12CC[C@H](N1)[C@H]1[C@H](C)Oc3nc(-c4cc(N)cc5ccc(F)c(CC)c45)c(F)c4c3c(nc(=O)n4CC(C)(CO)N3CCCC3)N1C2 ; [2H]C([2H])([2H])[C@]12CC[C@H](N1)[C@H]1[C@H](C)Oc3nc(-c4cc(N)cc5ccc(F)c(CC)c45)c(F)c4c3c(nc(=O)n4CC(C)(CO)N3CCCCC3)N1C2 ; [2H]C([2H])([2H])[C@]12CC[C@H](N1)[C@H]1[C@H](C)Oc3nc(-c4cc(N)c(F)c(C)c4C(F)(F)F)c(F)c4c3c(nc(=O)n4CC(C)(CO)N3CCC3)N1C2 ; [2H]C([2H])([2H])[C@]12CC[C@H](N1)[C@H]1[C@H](C)Oc3nc(-c4cc(N)nc(C)c4C(F)(F)F)c(F)c4c3c(nc(=O)n4CC(C)(CO)N3CCC3)N1C2]

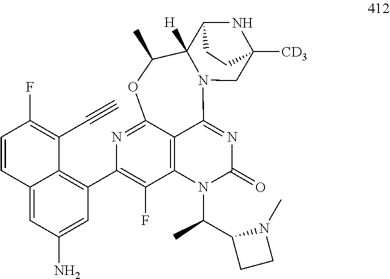 [SMILES: [2H]C([2H])([2H])[C@]12CC[C@H](N1)[C@H]1[C@H](C)Oc3nc(-c4cc(N)cc5ccc(F)c(C#C)c45)c(F)c4c3c(nc(=O)n4[C@H](C)[C@H]3CCN3C)N1C2]

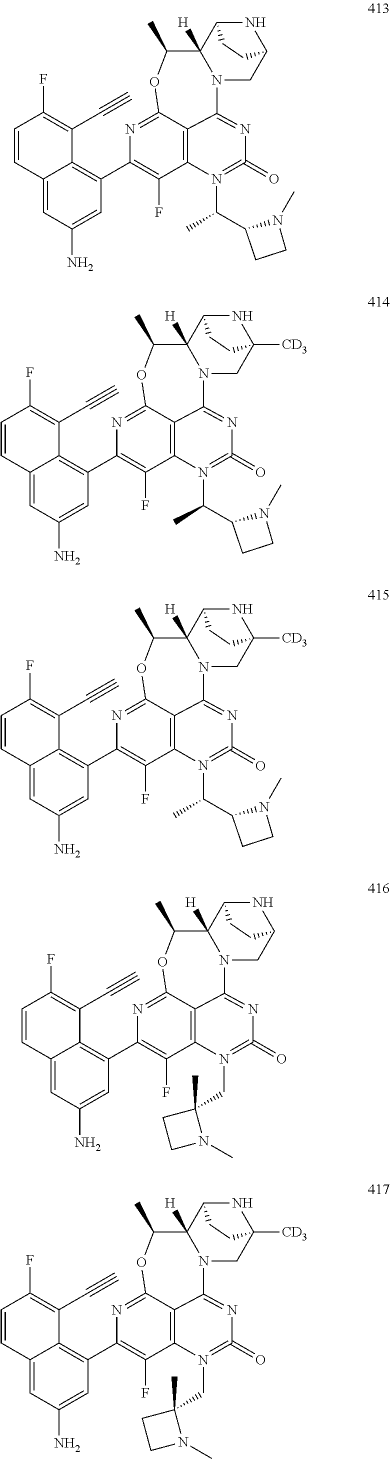

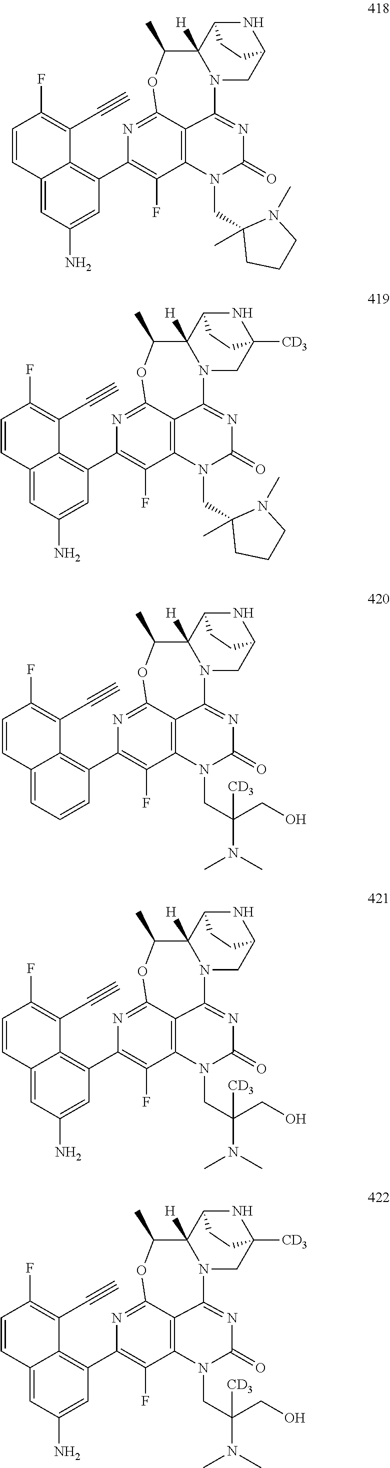

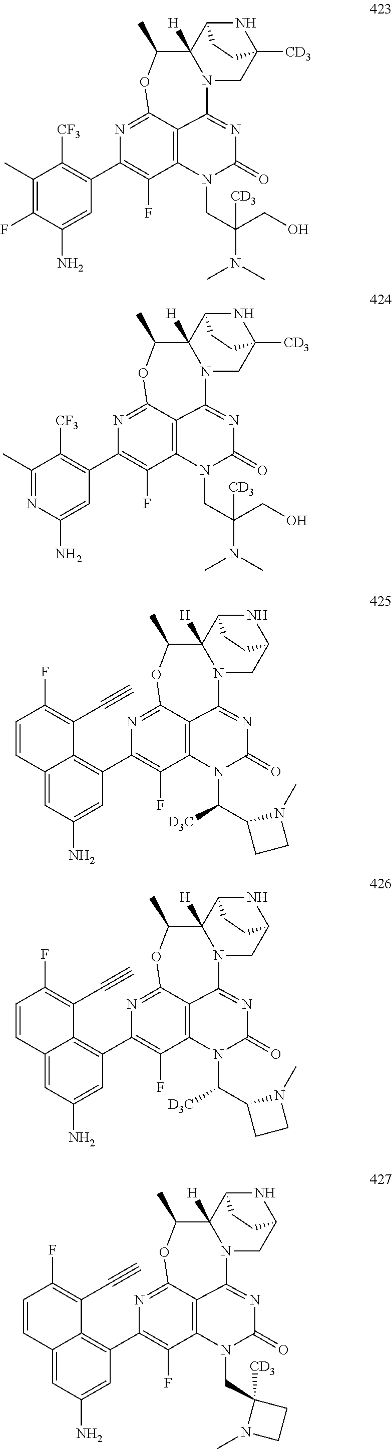

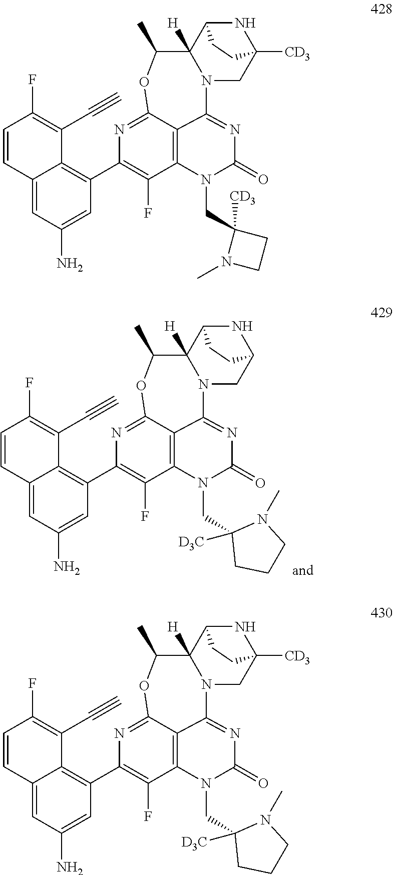 [SMILES: [2H]C([2H])([2H])[C@]12CC[C@H](N1)[C@H]1[C@H](C)Oc3nc(-c4cc(N)cc5ccc(F)c(C#C)c45)c(F)c4c3c(nc(=O)n4C[C@@]3(C([2H])([2H])[2H])CCN3C)N1C2 ; [2H]C([2H])([2H])[C@]1(Cn2c(=O)nc3c4c(nc(-c5cc(N)cc6ccc(F)c(C#C)c56)c(F)c42)O[C@@H](C)[C@@H]2[C@@H]4CC[C@H](CN32)N4)CCCN1C ; [2H]C([2H])([2H])[C@]12CC[C@H](N1)[C@H]1[C@H](C)Oc3nc(-c4cc(N)cc5ccc(F)c(C#C)c45)c(F)c4c3c(nc(=O)n4C[C@@]3(C([2H])([2H])[2H])CCCN3C)N1C2]

or a pharmaceutically acceptable salt thereof.

Compounds provided herein are described with reference to both generic formulae and specific compounds. In addition, the compounds of the present disclosure may exist in a number of different forms or derivatives, including but not limited to prodrugs, soft drugs, active metabolic derivatives (active metabolites), and their pharmaceutically acceptable salts, all within the scope of the present disclosure.

As used herein, the term “prodrugs” refers to compounds or pharmaceutically acceptable salts thereof which, when metabolized under physiological conditions or when converted by solvolysis, yield the desired active compound. Prodrugs include, without limitation, esters, amides, carbamates, carbonates, ureides, solvates, or hydrates of the active compound. Typically, the prodrug is inactive, or less active than the active compound, but may provide one or more advantageous handling, administration, and/or metabolic properties. For example, some prodrugs are esters of the active compound; during metabolysis, the ester group is cleaved to yield the active drug. Also, some prodrugs are activated enzymatically to yield the active compound, or a compound which, upon further chemical reaction, yields the active compound. Prodrugs may proceed from prodrug form to active form in a single step or may have one or more intermediate forms which may themselves have activity or may be inactive. Preparation and use of prodrugs is discussed in T. Higuchi and V. Stella, “Pro-drugs as Novel Delivery Systems”, Vol. 14 of the A.C.S. Symposium Series, in Bioreversible Carriers in Drug Design, ed. Edward B. Roche, American Pharmaceutical Association and Pergamon Press, 1987; in Prodrugs: Challenges and Rewards, ed. V. Stella, R. Borchardt, M. Hageman, R. Oliyai, H. Maag, J. Tilley, Springer-Verlag New York, 2007, all of which are hereby incorporated by reference in their entirety.

As used herein, the term “soft drug” refers to compounds that exert a pharmacological effect but break down to inactive metabolites degradants so that the activity is of limited time. See, for example, “Soft drugs: Principles and methods for the design of safe drugs”, Nicholas Bodor, Medicinal Research Reviews, Vol. 4, No. 4, 449-469, 1984, which is hereby incorporated by reference in its entirety.

As used herein, the term “metabolite”, e.g., active metabolite overlaps with prodrug as described above. Thus, such metabolites are pharmacologically active compounds or compounds that further metabolize to pharmacologically active compounds that are derivatives resulting from metabolic process in the body of a subject. For example, such metabolites may result from oxidation, reduction, hydrolysis, amidation, deamidation, esterification, deesterification, enzymatic cleavage, and the like, of the administered compound or salt or prodrug. Of these, active metabolites are such pharmacologically active derivative compounds. For prodrugs, the prodrug compound is generally inactive or of lower activity than the metabolic product. For active metabolites, the parent compound may be either an active compound or may be an inactive prodrug.

Prodrugs and active metabolites may be identified using routine techniques know in the art. See, e.g., Bertolini et al, 1997, J Med Chem 40:2011-2016; Shan et al., J Pharm Sci 86:756-757; Bagshawe, 1995, Drug Dev Res 34:220-230; Wermuth, supra.

As used herein, the term “pharmaceutically acceptable” indicates that the substance or composition is compatible chemically and/or toxicologically, with the other ingredients comprising a formulation, and/or the subjects being treated therewith.

As used herein, the term “pharmaceutically acceptable salt”, unless otherwise indicated, includes salts that retain the biological effectiveness of the free acids and bases of the specified compound and that are not biologically or otherwise undesirable. Contemplated pharmaceutically acceptable salt forms include, but are not limited to, mono, bis, tris, tetrakis, and so on. Pharmaceutically acceptable salts are non-toxic in the amounts and concentrations at which they are administered. The preparation of such salts can facilitate the pharmacological use by altering the physical characteristics of a compound without preventing it from exerting its physiological effect. Useful alterations in physical properties include lowering the melting point to facilitate transmucosal administration and increasing the solubility to facilitate administering higher concentrations of the drug.

Pharmaceutically acceptable salts include acid addition salts such as those containing sulfate, chloride, hydrochloride, fumarate, maleate, phosphate, sulfamate, acetate, citrate, lactate, tartrate, methanesulfonate, ethanesulfonate, benzenesulfonate, p-toluenesulfonate, cyclohexylsulfamate and quinate. Pharmaceutically acceptable salts can be obtained from acids such as hydrochloric acid, maleic acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, fumaric acid, and quinic acid.

Pharmaceutically acceptable salts also include basic addition salts such as those containing benzathine, chloroprocaine, choline, diethanolamine, ethanolamine, t-butylamine, ethylenediamine, meglumine, procaine, aluminum, calcium, lithium, magnesium, potassium, sodium, ammonium, alkylamine, and zinc, when acidic functional groups, such as carboxylic acid or phenol are present. For example, see Remington's Pharmaceutical Sciences, 19^{th} ed., Mack Publishing Co., Easton, PA, Vol. 2, p. 1457, 1995; “Handbook of Pharmaceutical Salts: Properties, Selection, and Use” by Stahl and Wermuth, Wiley-VCH, Weinheim, Germany, 2002. Such salts can be prepared using the appropriate corresponding bases.

Pharmaceutically acceptable salts can be prepared by standard techniques. For example, the free-base form of a compound can be dissolved in a suitable solvent, such as an aqueous or aqueous-alcohol solution containing the appropriate acid and then isolated by evaporating the solution. Thus, if the particular compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

Similarly, if the particular compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as L-glycine, L-lysine, and L-arginine, ammonia, primary, secondary, and tertiary amines, and cyclic amines, such as hydroxyethylpyrrolidine, piperidine, morpholine or piperazine, and inorganic salts derived from sodium, calcium, potassium, magnesium, manganese, iron, copper, zinc, aluminum and lithium.

It is also to be understood that the compounds of present disclosure can exist in unsolvated forms, solvated forms (e.g., hydrated forms), and solid forms (e.g., crystal or polymorphic forms), and the present disclosure is intended to encompass all such forms.

As used herein, the term “solvate” or “solvated form” refers to solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O. Examples of solvents that form solvates include, but are not limited to, water, isopropanol, ethanol, methanol, DMSO, ethyl acetate, acetic acid, and ethanolamine.

As used herein, the terms “crystal form”, “crystalline form”, “polymorphic forms” and “polymorphs” can be used interchangeably, and mean crystal structures in which a compound (or a salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compounds can be prepared by crystallization under different conditions.

The compounds of the present disclosure also include all isotopic forms thereof. “Isotopic form” of a compound indicates that atom(s) in the compound is substituted by isotope(s) of such atom(s). Isotopes of an atom include atoms having the same atomic number but different mass numbers. Examples of an isotope which can be incorporated into the compounds of the present disclosure include isotopes of hydrogen, carbon, nitrogen, oxygen, phosphorous, sulfur, fluorine, chlorine, bromide or iodine, such as but not limited to ¹H, ²H, ³H, ¹¹C, ¹²C, ¹³C, ¹⁴C, ¹⁴N, ¹⁵N, ¹⁶O, ¹⁷O, ¹⁸O, ³¹P, ³²P, ³²S, ³³S, ³⁴S, ³⁶S, ¹⁷F, ¹⁸F, ¹⁹F, ³⁵Cl, ³⁷Cl, ⁷⁹Br, ⁸¹Br, ¹²⁴I, ¹²⁷I and ¹³¹I. In some embodiments, hydrogen in the compounds provided herein includes protium, deuterium and tritium. In some embodiments, carbon in the compounds provided herein includes ¹²C and ¹³C.

Those of skill in the art will appreciate that compounds of the present disclosure may exist in different tautomeric forms, and all such forms are embraced within the scope of the present disclosure. The term “tautomer” or “tautomeric form” refers to structural isomers of different energies which are interconvertible via a low energy barrier. The presence and concentrations of the isomeric forms will depend on the environment the compound is found in and may be different depending upon, for example, whether the compound is a solid or is in an organic or aqueous solution. By way of examples, proton tautomers (also known as prototropic tautomers) include interconversions via migration of a proton, such as keto-enol, amide-imidic acid, lactam-lactim, imine-enamine isomerizations and annular forms where a proton can occupy two or more positions of a heterocyclic system. Valence tautomers include interconversions by reorganization of some of the bonding electrons. Tautomers can be in equilibrium or sterically locked into one form by appropriate substitution. Compounds of the present disclosure identified by name or structure as one particular tautomeric form are intended to include other tautomeric forms unless otherwise specified.

### Synthesis of Compounds

The compounds provided herein can be prepared using any known organic synthesis techniques and can be synthesized according to any of numerous possible synthetic routes

Reactions for preparing compounds of the present disclosure can be carried out in suitable solvents, which can be readily selected by one skilled in the art of organic synthesis. Suitable solvents can be substantially non-reactive with starting materials (reactants), intermediates, or products at the temperatures at which the reactions are carried out, e.g. temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected by one skilled in the art.

Preparation of compounds of the present disclosure can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3rd Ed., Wiley & Sons, Inc., New York (1999), in P. Kocienski, Protecting Groups, Georg Thieme Verlag, 2003, and in Peter G. M. Wuts, Greene's Protective Groups in Organic Synthesis, 5^{th} Edition, Wiley, 2014, all of which are incorporated herein by reference in its entirety.

Reactions can be monitored according to any suitable method known in the art. For example, product formation can be monitored by spectroscopic means, such as nuclear magnetic resonance spectroscopy (e.g. ¹H or ¹³C), infrared spectroscopy, spectrophotometry (e.g. UV-visible), mass spectrometry, or by chromatographic methods such as high performance liquid chromatography (HPLC), liquid chromatography-mass spectroscopy (LCMS), or thin layer chromatography (TLC). Compounds can be purified by one skilled in the art by a variety of methods, including high performance liquid chromatography (HPLC) (“Preparative LC-MS Purification: Improved Compound Specific Method Optimization” Karl F. Blom, Brian Glass, Richard Sparks, Andrew P. Combs J. Combi. Chem. 2004, 6(6), 874-883, which is incorporated herein by reference in its entirety), and normal phase silica chromatography.

### Use of Compounds

In an aspect, the present disclosure provides compounds capable of inhibiting KRAS protein, in particular KRAS G12D protein.

As used herein, the term “therapy” is intended to have its normal meaning of dealing with a disease in order to entirely or partially relieve one, some or all of its symptoms, or to correct or compensate for the underlying pathology, thereby achieving beneficial or desired clinical results. For purposes of this disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Therapy” can also mean prolonging survival as compared to expected survival if not receiving it. Those in need of therapy include those already with the condition or disorder as well as those prone to have the condition or disorder or those in which the condition or disorder is to be prevented. The term “therapy” also encompasses prophylaxis unless there are specific indications to the contrary. The terms “therapeutic” and “therapeutically” should be interpreted in a corresponding manner.

As used herein, the term “prophylaxis” is intended to have its normal meaning and includes primary prophylaxis to prevent the development of the disease and secondary prophylaxis whereby the disease has already developed and the patient is temporarily or permanently protected against exacerbation or worsening of the disease or the development of new symptoms associated with the disease.

The term “treatment” is used synonymously with “therapy”. Similarly the term “treat” can be regarded as “applying therapy” where “therapy” is as defined herein.

In a further aspect, the present disclosure provides use of the compound of the present disclosure or a pharmaceutically acceptable salt thereof or the pharmaceutical composition of the present disclosure for use in therapy, for example, for use in therapy associated with KRAS protein, in particular, in therapy associated with KRAS G12D protein.

In a further aspect, the present disclosure provides use of the compound of the present disclosure or a pharmaceutically acceptable salt thereof or the pharmaceutical composition of the present disclosure, in the manufacture of a medicament for treating cancer.

In some embodiments, the cancer is mediated by KRAS protein. In some embodiments, the cancer is mediated by KRAS G12D protein.

### Pharmaceutical Compositions

In a further aspect, there is provided pharmaceutical compositions comprising one or more compounds of the present disclosure, or a pharmaceutically acceptable salt thereof.

In another aspect, there is provided pharmaceutical composition comprising one or more compounds of the present disclosure, or a pharmaceutically acceptable salt thereof, and at least one pharmaceutical acceptable excipient.

As used herein, the term “pharmaceutical composition” refers to a formulation containing the molecules or compounds of the present disclosure in a form suitable for administration to a subject.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used herein includes both one and more than one such excipient. The term “pharmaceutically acceptable excipient” also encompasses “pharmaceutically acceptable carrier” and “pharmaceutically acceptable diluent”.

The particular excipient used will depend upon the means and purpose for which the compounds of the present disclosure is being applied. Solvents are generally selected based on solvents recognized by persons skilled in the art as safe to be administered to a mammal including humans. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof.

In some embodiments, suitable excipients may include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

In some embodiments, suitable excipients may include one or more stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present disclosure or pharmaceutical composition thereof) or aid in the manufacturing of the pharmaceutical product (i.e., medicament). The active pharmaceutical ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980). A “liposome” is a small vesicle composed of various types of lipids, phospholipids and/or surfactant which is useful for delivery of a drug (such as the compounds disclosed herein and, optionally, a chemotherapeutic agent) to a mammal including humans. The components of the liposome are commonly arranged in a bilayer formation, similar to the lipid arrangement of biological membranes.

The pharmaceutical compositions provided herein can be in any form that allows for the composition to be administered to a subject, including, but not limited to a human, and formulated to be compatible with an intended route of administration.

A variety of routes are contemplated for the pharmaceutical compositions provided herein, and accordingly the pharmaceutical composition provided herein may be supplied in bulk or in unit dosage form depending on the intended administration route. For example, for oral, buccal, and sublingual administration, powders, suspensions, granules, tablets, pills, capsules, gelcaps, and caplets may be acceptable as solid dosage forms, and emulsions, syrups, elixirs, suspensions, and solutions may be acceptable as liquid dosage forms. For injection administration, emulsions and suspensions may be acceptable as liquid dosage forms, and a powder suitable for reconstitution with an appropriate solution as solid dosage forms. For inhalation administration, solutions, sprays, dry powders, and aerosols may be acceptable dosage form. For topical (including buccal and sublingual) or transdermal administration, powders, sprays, ointments, pastes, creams, lotions, gels, solutions, and patches may be acceptable dosage form. For vaginal administration, pessaries, tampons, creams, gels, pastes, foams and spray may be acceptable dosage form.

The quantity of active ingredient in a unit dosage form of composition is a therapeutically effective amount and is varied according to the particular treatment involved. As used herein, the term “therapeutically effective amount” refers to an amount of a molecule, compound, or composition comprising the molecule or compound to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; the rate of administration; the therapeutic or combination of therapeutics selected for administration; and the discretion of the prescribing physician. Therapeutically effective amounts for a given situation can be determined by routine experimentation that is within the skill and judgment of the clinician.

In some embodiments, the pharmaceutical compositions of the present disclosure may be in a form of formulation for oral administration.

In certain embodiments, the pharmaceutical compositions of the present disclosure may be in the form of tablet formulations. Suitable pharmaceutically-acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case using conventional coating agents and procedures well known in the art.

In certain embodiments, the pharmaceutical compositions of the present disclosure may be in a form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

In certain embodiments, the pharmaceutical compositions of the present disclosure may be in the form of aqueous suspensions, which generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), coloring agents, flavoring agents, and/or sweetening agents (such as sucrose, saccharine or aspartame).

In certain embodiments, the pharmaceutical compositions of the present disclosure may be in the form of oily suspensions, which generally contain suspended active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an anti-oxidant such as ascorbic acid.

In certain embodiments, the pharmaceutical compositions of the present disclosure may be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring and preservative agents.

In certain embodiments, the pharmaceutical compositions provided herein may be in the form of syrups and elixirs, which may contain sweetening agents such as glycerol, propylene glycol, sorbitol, aspartame or sucrose, a demulcent, a preservative, a flavoring and/or coloring agent.

In some embodiments, the pharmaceutical compositions of the present disclosure may be in a form of formulation for injection administration.

In certain embodiments, the pharmaceutical compositions of the present disclosure may be in the form of a sterile injectable preparation, such as a sterile injectable aqueous or oleaginous suspension. This suspension may be formulated according to the known art using those suitable dispersing or wetting agents and suspending agents, which have been mentioned above. The sterile injectable preparation may also be a sterile injectable solution or suspension in a non-toxic parenterally acceptable diluent or solvent, such as a solution in 1,3-butanediol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

In some embodiments, the pharmaceutical compositions of the present disclosure may be in a form of formulation for inhalation administration.

In certain embodiments, the pharmaceutical compositions of the present disclosure may be in the form of aqueous and nonaqueous (e.g., in a fluorocarbon propellant) aerosols containing any appropriate solvents and optionally other compounds such as, but not limited to, stabilizers, antimicrobial agents, antioxidants, pH modifiers, surfactants, bioavailability modifiers and combinations of these. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols.

In some embodiments, the pharmaceutical compositions of the present disclosure may be in a form of formulation for topical or transdermal administration.

In certain embodiments, the pharmaceutical compositions provided herein may be in the form of creams, ointments, gels and aqueous or oily solutions or suspensions, which may generally be obtained by formulating an active ingredient with a conventional, topically acceptable excipients such as animal and vegetable fats, oils, waxes, paraffins, starch, tragacanth, cellulose derivatives, polyethylene glycols, silicones, bentonites, silicic acid, talc and zinc oxide, or mixtures thereof.

In certain embodiments, the pharmaceutical compositions provided herein may be formulated in the form of transdermal skin patches that are well known to those of ordinary skill in the art.

Besides those representative dosage forms described above, pharmaceutically acceptable excipients and carriers are generally known to those skilled in the art and are thus included in the present disclosure. Such excipients and carriers are described, for example, in “Remingtons Pharmaceutical Sciences” Mack Pub. Co., New Jersey (1991), in “Remington: The Science and Practice of Pharmacy”, Ed. University of the Sciences in Philadelphia, 21^{st} Edition, LWW (2005), which are incorporated herein by reference.

In some embodiments, the pharmaceutical compositions of the present disclosure can be formulated as a single dosage form. The amount of the compounds provided herein in the single dosage form will vary depending on the subject treated and particular mode of administration.

In some embodiments, the pharmaceutical compositions of the present disclosure can be formulated so that a dosage of between 0.001-1000 mg/kg body weight/day, for example, 0.01-800 mg/kg body weight/day, 0.01-700 mg/kg body weight/day, 0.01-600 mg/kg body weight/day, 0.01-500 mg/kg body weight/day, 0.01-400 mg/kg body weight/day, 0.01-300 mg/kg body weight/day, 0.1-200 mg/kg body weight/day, 0.1-150 mg/kg body weight/day, 0.1-100 mg/kg body weight/day, 0.5-100 mg/kg body weight/day, 0.5-80 mg/kg body weight/day, 0.5-60 mg/kg body weight/day, 0.5-50 mg/kg body weight/day, 1-50 mg/kg body weight/day, 1-45 mg/kg body weight/day, 1-40 mg/kg body weight/day, 1-35 mg/kg body weight/day, 1-30 mg/kg body weight/day, 1-25 mg/kg body weight/day of the compounds provided herein, or a pharmaceutically acceptable salt thereof, can be administered. In some instances, dosage levels below the lower limit of the aforesaid range may be more than adequate, while in other cases still larger doses may be employed without causing any harmful side effect, provided that such larger doses are first divided into several small doses for administration throughout the day. For further information on routes of administration and dosage regimes, see Chapter 25.3 in Volume 5 of Comprehensive Medicinal Chemistry (Corwin Hansch; Chairman of Editorial Board), Pergamon Press 1990, which is specifically incorporated herein by reference.

In some embodiments, the pharmaceutical compositions of the present disclosure can be formulated as short-acting, fast-releasing, long-acting, and sustained-releasing. Accordingly, the pharmaceutical formulations of the present disclosure may also be formulated for controlled release or for slow release.

In a further aspect, there is also provided veterinary compositions comprising one or more molecules or compounds of the present disclosure or pharmaceutically acceptable salts thereof and a veterinary carrier. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

The pharmaceutical compositions or veterinary compositions may be packaged in a variety of ways depending upon the method used for administering the drug. For example, an article for distribution can include a container having deposited therein the compositions in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings. The compositions may also be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described.

In a further aspect, there is also provided pharmaceutical compositions comprise one or more compounds of the present disclosure, or a pharmaceutically acceptable salt thereof, as a first active ingredient, and a second active ingredient.

In some embodiments, the second active ingredient has complementary activities to the compound provided herein such that they do not adversely affect each other. Such ingredients are suitably present in combination in amounts that are effective for the purpose intended.

### Method of Treatment of Disease

In a further aspect, the present disclosure provides a method for treating cancer, comprising administering an effective amount of the compound or a pharmaceutically acceptable salt thereof or the pharmaceutical composition provided herein to a subject in need thereof.

In some embodiments, the compounds or pharmaceutically acceptable salts thereof and the compositions provided herein may be used for the treatment of a KRAS G12D-associated cancer in a subject in need thereof, comprising administering to said subject a therapeutically effective amount of a compound provided herein, a pharmaceutically acceptable salt thereof, or a pharmaceutical composition comprising the compound or pharmaceutically acceptable salt thereof.

In some embodiments, the compounds or pharmaceutically acceptable salts thereof and the compositions provided herein may be used for the treatment of a wide variety of cancers including tumors such as lung, prostate, breast, brain, skin, cervical carcinomas, testicular carcinomas, etc. More particularly, cancers that may be treated by the compounds or pharmaceutically acceptable salts thereof and the compositions provided herein include, but are not limited to tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, the compounds or pharmaceutically acceptable salts thereof and the compositions provided herein can be used to treat:
(i) Cardiac: sarcoma (angiosarcoma, fibrosarcoma, rhabdomyosarcoma, liposarcoma), myxoma, rhabdomyoma, fibroma, lipoma and teratoma;
(ii) Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma;
(iii) Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma);
(iv) Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma);
(v) Liver: hepatoma (hepatocellular carcinoma), cholangiocarcinoma, hepatoblastoma, angiosarcoma, hepatocellular adenoma, hemangioma;
(vi) Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors;
(vii) Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma);
(viii) Gynecological: uterus (endometrial ‘carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma);
(ix) Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma);
(x) Skin: malignant melanoma, basal cell carcinoma, squamous cell carcinoma, Kaposi's sarcoma, moles dysplastic nevi, lipoma, angioma, dermatofibroma, keloids, psoriasis; and
(xi) Adrenal glands: neuroblastoma.

In certain embodiments, the cancer that can be treated with the compounds or pharmaceutically acceptable salts thereof and the compositions provided herein is non-small cell lung cancer, small cell lung cancer, colorectal cancer, rectal cancer or pancreatic cancer.

The concentration and route of administration to the subject will vary depending on the cancer to be treated. In certain embodiments, the administering is conducted via a route selected from the group consisting of parenteral, intraperitoneal, intradermal, intracardiac, intraventricular, intracranial, intracerebrospinal, intrasynovial, intrathecal administration, intramuscular injection, intravitreous injection, intravenous injection, intra-arterial injection, oral, buccal, sublingual, transdermal, topical, intratracheal, intrarectal, subcutaneous, and topical administration.

The compounds, pharmaceutically acceptable salts thereof and pharmaceutical compositions comprising such compounds and salts also may be co-administered with other anti-neoplastic compounds, e.g., chemotherapy, or used in combination with other treatments, such as radiation or surgical intervention, either as an adjuvant prior to surgery or post-operatively.

In some embodiments, the compounds, pharmaceutically acceptable salts thereof and pharmaceutical compositions comprising such compounds and salts can be administered simultaneously, separately or sequentially with one or more additional therapeutic agents. In certain embodiments, the additional therapeutic agent is selected from an anti-PD-1 antagonist, an MEK inhibitor, a SHP2 inhibitor, a platinum agent or pemetrexed. In certain embodiments, the anti-PD-1 antagonist is selected from nivolumab, pembrolizumab, or AMB 404. In certain embodiments, the MEK inhibitor is trametinib. In certain embodiments, the SHP2 inhibitor is RMC-4630.

In another aspect, the present disclosure also provides a method for treating cancer in a subject in need thereof, the method comprising:
(a) acquiring the knowledge that the cancer is associated with KRAS G12D mutation; and
(b) administering to the subject an effective amount of a compound or a pharmaceutically acceptable salt thereof or the pharmaceutical composition of the present disclosure.

In another aspect, the present disclosure provides a method for inhibiting KRAS G12D activity in a subject in need thereof, comprising administering the compound or a pharmaceutically acceptable salt thereof or the pharmaceutical composition of the present disclosure to the subject.

### EXAMPLES

For the purpose of illustration, the following examples are included. However, it is to be understood that these examples do not limit the present disclosure and are only meant to suggest a method of practicing the present disclosure.

### Intermediate 1: tert-butyl (1S,2S,5R)-2-(hydroxymethyl)-3,8-diazabicyclo[3.2.1]octane-8-carboxylate

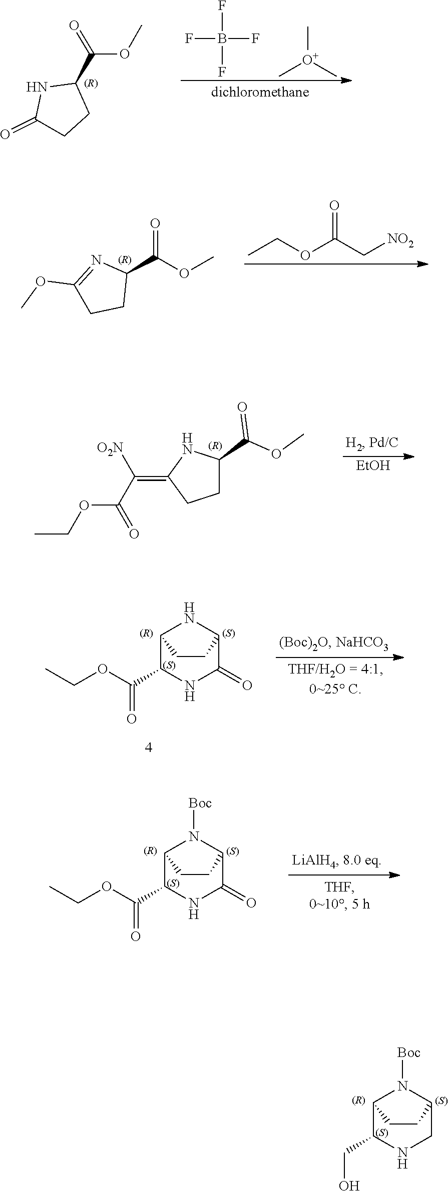

### Step 1. methyl (2R)-5-methoxy-3,4-dihydro-2H-pyrrole-2-carboxylate

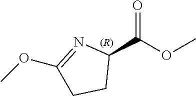

To a solution of methyl (2R)-5-oxopyrrolidine-2-carboxylate (88 g, 0.61 mol) in DCM (1 L) were added trimethyloxonium tetrafluoroborate (100 g, 0.67 mol), and the reaction mixture was stirred at room temperature for 18 hrs. The reaction mixture was quenched with saturated NaHCO₃ solution at 0° C. The organic layer was separated, washed with further saturated NaHCO₃ solution, and concentrated in vacuum. The residue was purified by flash chromatography (silica gel, 0-30% EtOAc in PE) to afford methyl (2R)-5-methoxy-3,4-dihydro-2H-pyrrole-2-carboxylate (69 g, 71% yield) as a yellow oil. MS ESI (m/z): 158 [M+H]⁺

### Step 2. methyl (2R,5Z)-5-(2-ethoxy-1-nitro-2-oxoethylidene)pyrrolidine-2-carboxylate

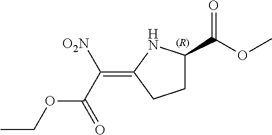

To a flask containing methyl (2R)-5-methoxy-3,4-dihydro-2H-pyrrole-2-carboxylate (69 g, 0.44 mol) was added ethyl 2-nitroacetate (38.9 mL, 0.35 mol) at room temperature. The mixture was stirred at 60° C. for 18 hrs. The results concentrated in vacuum. The residue was purified by flash chromatography (silica gel, 0-25% EtOAc in PE) to provide methyl (2R,5Z)-5-(2-ethoxy-1-nitro-2-oxoethylidene)pyrrolidine-2-carboxylate (37 g, 33% yield) as a yellow oil. MS ESI (m/z): 259 [M+H]⁺.

### Step 3. ethyl (1S,2S,5R)-4-oxo-3,8-diazabicyclo[3.2.1]octane-2-carboxylate

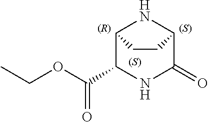

To a solution of methyl (2R,5Z)-5-(2-ethoxy-1-nitro-2-oxoethylidene)pyrrolidine-2-carboxylate (32 g, 0.12 mol) in EtOH (200 mL) was added Pd/C (10 g, 10% wt). The reaction mixture was degassed under H₂ balloon for three times, and stirred at 50° C. under H₂ balloon for 20 hrs, then 80° C. for further 48 hrs. The mixture was filtered and the filtrate was concentrated in vacuum. The residue was purified by flash chromatography (silica gel, 0-10% MeOH in DCM) to give ethyl (1S,2S,5R)-4-oxo-3,8-diazabicyclo[3.2.1]octane-2-carboxylate (10 g, 41% yield) as a white solid. MS ESI (m/z): 199 [M+H]⁺.

### Step 4. 8-tert-butyl 2-ethyl (1S,2S,5R)-4-oxo-3,8-diazabicyclo[3.2.1]octane-2,8-dicarboxylate

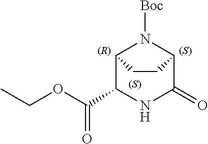

To a solution of ethyl (1S,2S,5R)-4-oxo-3,8-diazabicyclo[3.2.1]octane-2-carboxylate (1.54 g, 7.77 mmol) in THF (20 mL) and H₂O (5 mL) were added NaHCO₃ (2.20 g, 10.1 mmol) and Boc₂O (2.2 g, 10.1 mmol), and the reaction was stirred at room temperature for 16 hrs. The reaction was partitioned with EtOAc and water. The organic layer was separated, washed with brine, dried with anhydrous Na₂SO₄, filtered and concentrated to dryness. The residue was purified by flash chromatography (silica gel, 0-10% MeOH in DCM) to give 8-tert-butyl 2-ethyl (1S,2S,5R)-4-oxo-3,8-diazabicyclo[3.2.1]octane-2,8-dicarboxylate (1.9 g, 81% yield) as a white solid. MS ESI (m/z): 299 [M+H]⁺

### Step 5. tert-butyl (1S,2S,5R)-2-(hydroxymethyl)-3,8-diazabicyclo[3.2.1]octane-8-carboxylate

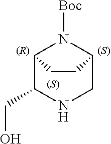

To a suspension of LiAlH₄ (1.91 g, 50.4 mmol) in THF (20 mL) was added 8-tert-butyl 2-ethyl (1R,2R,5S)-4-oxo-3,8-diazabicyclo[3.2.1]octane-2,8-dicarboxylate (1.88 g, 6.30 mmol) in THF (20 mL) drop-wisely at 0° C. The reaction mixture was stirred at 0° C. for 5 hrs under N₂ atmosphere. The reaction was quenched successively with water (1.9 mL), aq NaOH (1.9 mL, 15% wt) and water (5.7 mL). The mixture was then stirred for 30 min and filtered. The filter cake was washed with DCM/MeOH (10/1) and concentrated to give the residue which was purified by flash chromatography (silica gel, 0-15% MeOH in DCM) to give t tert-butyl (1S,2S,5R)-2-(hydroxymethyl)-3,8-diazabicyclo[3.2.1]octane-8-carboxylate (900 mg, 59% yield) as a yellow oil. MS (ESI) m/z: 243 (M+H)⁺.

### Intermediate 2: tert-butyl (5S,5aS,6S,9R)-2-chloro-1-fluoro-13-hydroxy-5-methyl-5a,6,7,8,9,10-hexahydro-5H-6,9-epiminoazepino[2′,1′:3,4][1,4]oxazepino[5,6,7-de][1,6]naphthyridine-15-carboxylate

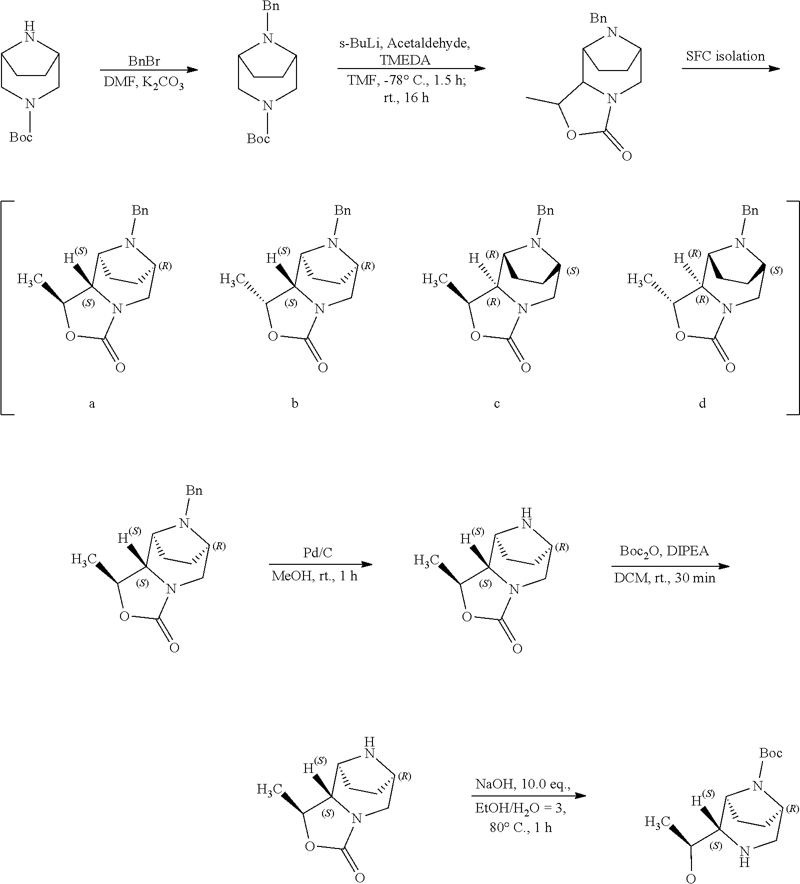

### Step 1. tert-butyl (1R,5S)-8-benzyl-3,8-diazabicyclo[3.2.1]octane-3-carboxylate

To a solution of tert-butyl (1R,5S)-3,8-diazabicyclo[3.2.1]octane-3-carboxylate (25 g, 117.8 mmol) in DMF (200 mL) was added (bromomethyl)benzene (23.2 g, 135 mmol) and K₂CO₃ (32.5 g, 235.5 mmol). The reaction was stirred at 40° C. for 12 hours. The reaction was concentrated. H₂O (50 mL) was added. The aqueous phase was extracted with BA (3×75 mL). The combined organics were dried over anhydrous Na₂SO₄, filtered and concentrated in vacuo. The resulting oil was purified by flash column chromatography with a gradient elution of hexane (920%) and EtOAc (80%) to hexanes (85%) and EtOAc (15%) to provide tert-butyl (1R,5S)-8-benzyl-3,8-diazabicyclo[3.2.1]octane-3-carboxylate (22.5 g, 63.2% yield) as a yellow-white solid.

LC/MS (ESI) (m/z): 303 [M+H]⁺.

### Step 2. (6S,9R)-10-benzyl-1-methylhexahydro-1H,3H-6,9-epiminooxazolo[3,4-a]azepin-3-one

Under nitrogen, to a solution of tert-butyl (1R,5S)-8-benzyl-3,8-diazabicyclo[3.2.1]octane-3-carboxylate (9.0 g, 29.7 mmol) and TMEDA (6.92 g, 59.5 mmol) in diethyl ether (90 mL) was added s-BuLi (45.8 mL, 59.5 mmol, 1.3 M in hexane) dropwise at −78° C. The resulting solution was stirred for 1.5 h at −78° C. Then acetaldehyde (3.28 g, 74.3 mmol) was added at −78° C., The reaction was allowed to warm to room temperature gradually and stirred overnight. The mixture was quenched with NH₄Cl (aq.) and extracted with EtOAc. The combined organic layers were dried over anhydrous Na₂SO₄ and concentrated under vacuum. The residue was purified by flash chromatography on silica gel (0-50% EtOAc in petroleum ether) to yield 5.89 g mixture of 4 diastereoisomers.

### Step 3. (1S,6R,9S,9aS)-10-benzyl-1-methylhexahydro-1H,3H-6,9-epiminooxazolo[3,4-a]azepin-3-one

The products from the above reaction was separated by Prep-SFC (Column: ChiralPak AS, 300×mm, 10 μm; Mobile Phase A: CO₂, Mobile Phase B: IPA(0.1% NH3H2O); Flow rate: 200 mL/min; Gradient isocratic 40% B; Column Temperature (° C.): 38; Back Pressure (bar: 100; Wave Length: 220 nm; RT1 (min): 1.560; RT2 (min): 1.907; Sample Solvent: MeOH/DCM—Preparative; Injection Volume: 12 mL Number Of Runs: 30) to yield the compound a (1.800 g, 22.2% yield) (the first peak) and compound d (1.870 g, 23.1% yield) (the third peak) and mixture of compound b and c (the second peak). The mixture of compound b and c was re-separated by Prep-SFC (Column’: ChiralPak AD, 300×mm, 10 μm; Mobile Phase A: CO₂, Mobile Phase B: Ethanol; Flow rate: 200 mL/min; Gradient: isocratic 25% B; Column Temperature (° C.): 38; Back Pressure (bar): 100; Wave Length: 220 nm; RT1 (min): 2.007; RT2 (min): 2.374; Sample Solvent: MeOH/DCM—Preparative; Injection Volume: 10 mL; Number Of Runs: 30) to yield compound b (0.641 g, 7.9% yield) (the faster peak) and compound c (0.650 g, 11.7% yield) (the slower peak) as yellow solid. LC-MS: (ESI, my/z): [M+H]=273. Compound a is the desired isomer.

### Step 4. (1S,6R,9S,9aS)-1-methylhexahydro-1H,3H-6,9-epiminooxazolo[3,4-a]azepin-3-one

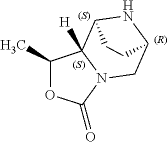

A solution of (1S,6R,9S,9aS)-10-benzyl-1-methylhexahydro-1H,3H-6,9-epiminooxazolo[3,4-a]azepin-3-one (820 mg, 3.01 mmol) and Pd/C (500 mg, 10%) in methyl alcohol (15 mL) was stirred for 1 hour at room temperature under an atmosphere of hydrogen gas. The catalyst was filtered off. The filtrate was concentrated under vacuum to yield (1S,6R,9S,9aS)-1-methylhexahydro-1H,3H-6,9-epiminooxazolo[3,4-a]azepin-3-one (540 mg, 98.4% yield) the title compound as a yellow oil which was used for next step without further purification.

LC/MS (ESI) (m/z): 183 [M+H]⁺.

### Step 5. tert-butyl (1S,6R,9S,9aS)-1-methyl-3-oxohexahydro-1H,3H-6,9-epiminooxazolo[3,4-a]azepine-10-carboxylate

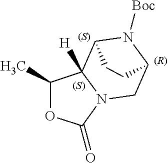

A solution of (1S,6R,9S,9aS)-1-methylhexahydro-1H,3H-6,9-epiminooxazolo[3,4-a]azepin-3-one (540 mg, 2.96 mmol), (Boc)₂O (966 mg, 4.43 mmol) and DIPEA (1.14 g, 8.85 mmol) in dichloromethane (10 mL) was stirred for 30 minutes at room temperature. The reaction system was quenched with water, extracted with DCM. The combined organic layers were dried over anhydrous Na₂SO₄ and concentrated under vacuum. The residue was purified by flash chromatography on silica gel (gradient: 0-100% EtOAc/petroleum ether) to yield the title compound tert-butyl (1S,6R,9S,9aS)-1-methyl-3-oxohexahydro-1H,3H-6,9-epiminooxazolo[3,4-a]azepine-10-carboxylate (550 mg, 1.94 mmol, 68.3% yield) as a white solid.

LC/MS (ESI) (m/z): 283 [M+H]⁺.

### Step 6. tert-butyl (1S,2S,5R)-2-((S)-1-hydroxyethyl)-3,8 diazabicyclo[3.2.1]octane-8-carboxylate

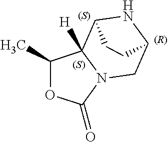

A solution of tert-butyl (1S,6R,9S,9aS)-1-methyl-3-oxohexahydro-1H,3H-6,9-epiminooxazolo[3,4-a]azepine-10-carboxylate (550 mg, 1.94 mmol) and NaOH (770 mg, 19.25 mmol) in ethanol (7.3 mL) and water (2.4 mL) was stirred for 1 hour at 80° C. The reaction solution was cooled to room temperature and diluted with water, extracted with DCM. The combined organic layers were dried over anhydrous Na₂SO₄ and concentrated under vacuum to yield the title compound tert-butyl (1S,2S,5R)-2-((S)-1-hydroxyethyl)-3,8 diazabicyclo[3.2.1]octane-8-carboxylate (470 mg, 1.82 mmol, 94.46% yield) as an oil which was used for next step without further purification.

LC/MS (ESI) (m/z): 257 [M+H]⁺.

### Intermediate 3: (2,6-dimethylenetetrahydro-1H-pyrrolizin-7a(5H)-yl)methanol

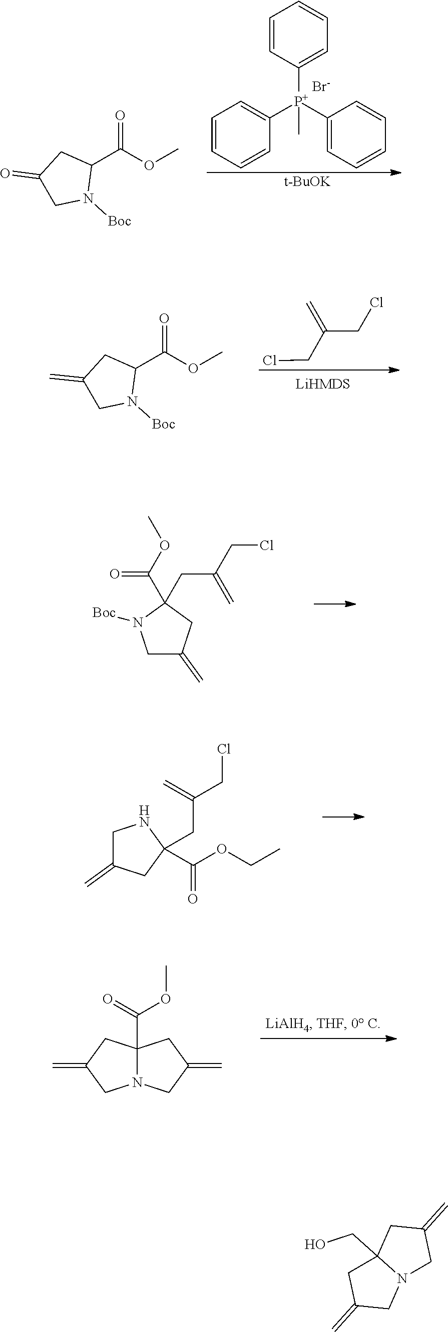

### Step 1. 1-(tert-butyl) 2-methyl 4-methylenepyrrolidine-1,2-dicarboxylate

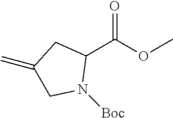

A solution of methyltriphenylphosphonium bromide (29.4 g, 82.2 mmol) in THF (150 ml) at 0° C. was potassium-t-butoxide (9.2 g, 82.2 mmol) in anhydrous THF (80 mL). After stirred at 0° C. for 2 h, the resulting yellow suspension was added the solution of 1-(tert-butyl) 2-methyl 4-oxopyrrolidine-1,2-dicarboxylate (10 g, 41.1 mmol) in THF (40 mL). After stirring at R.T for 1 h, the reaction mixture was concentrated, diluted with EtOAc, washed with water, brine, dried over Mg₂SO₄, concentrated purified by flash chromatography to afford 1-(tert-butyl) 2-methyl 4-methylenepyrrolidine-1,2-dicarboxylate (4 g, 40.3% yield).

### Step 2. 1-(tert-butyl) 2-methyl 2-(2-(chloromethyl)allyl)-4-methylenepyrrolidine-1,2-dicarboxylate

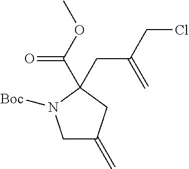 [SMILES: C=C(CCl)CC1(C(=O)OC)CC(=C)CN1C(=O)OC(C)(C)C]

A solution of 1-(tert-butyl) 2-methyl 4-methylenepyrrolidine-1,2-dicarboxylate (4.0 g, 16.6 mmol) in THF (30 mL) was added slowly to LiHMDS (33.2 mL, 33.2 mmol, 1.0M in THF) at −78° C. under nitrogen atmosphere. After stirring for 1 hour at −78° C., 3-chloro-2-(chloromethyl)prop-1-ene (5.2 g, 41.5 mmol) was added dropwise at −78° C. under nitrogen atmosphere. The resulting mixture was warmed up and then stirred for 16 hours at room temperature. The reaction mixture was diluted with water and then extracted with EtOAc. The combined organic layers were washed with water, brine, dried over anhydrous Na₂SO₄, filtered and then concentrated. The residue was purified by silica gel column chromatography to afford 1-(tert-butyl) 2-methyl 2-(2-(chloromethyl)allyl)-4-methylenepyrrolidine-1,2-dicarboxylate (4 g, 73.1% yield) as a yellow oil.

### Step 3. ethyl 2-(2-(chloromethyl)allyl)-4-methylenepyrrolidine-2-carboxylate

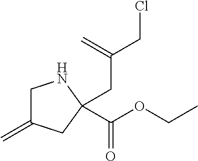 [SMILES: C=C(CCl)CC1(C(=O)OCC)CC(=C)CN1]

A solution of 1-(tert-butyl) 2-methyl 2-(2-(chloromethyl)allyl)-4-methylenepyrrolidine-1,2-dicarboxylate (4 g, 12.1 mmol) and HCl/dioxane (15 mL, 4N) was stirred for 2 h at room temperature. The reaction mixture was concentrated to afford ethyl 2-(2-(chloromethyl)allyl)-4-methylenepyrrolidine-2-carboxylate (2.8 g, 94.6% yield) as a white solid.

### Step 4. methyl 2,6-dimethylenetetrahydro-1H-pyrrolizine-7a(5H)-carboxylate

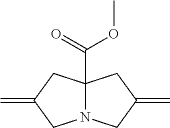

A mixture of ethyl 2-(2-(chloromethyl)allyl)-4-methylenepyrrolidine-2-carboxylate (2.8 g, 11.5 mmol) and NH₃/MeOH (20 mL, 7N) was stirred for 2 hours at room temperature. The reaction mixture was concentrated, and the residue was purified by silica gel column chromatography to afford methyl 2,6-dimethylenetetrahydro-1H-pyrrolizine-7a(5H)-carboxylate (2.0 g, 90% yield) as a white solid.

¹H NMR (400 MHz, CDCl₃) δ 4.99 (p, J=2.1 Hz, 2H), 4.96-4.92 (m, 2H), 3.82 (d, J=14.6 Hz, 2H), 3.74 (s, 3H), 3.28 (d, J=1.9 Hz, 1H), 3.24 (d, J=1.9 Hz, 1H), 3.01 (dd, J=2.0, 0.8 Hz, 1H), 2.97 (dd, J=1.9, 0.8 Hz, 1H), 2.60 (d, J=1.9 Hz, 1H), 2.56 (d, J=2.0 Hz, 1H)

### Step 5. (2,6-dimethylenetetrahydro-1H-pyrrolizin-7a(5H)-yl)methanol

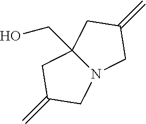

To a solution of methyl 2,6-dimethylenetetrahydro-1H-pyrrolizine-7a(5H)-carboxylate (2 g, 10.35 mmol) in THF (30 mL) was treated at 0° C. with LiAlH₄ (787 mg, 20.7 mmol), The reaction mixture was stirred at 0° C. for 2 hours under N₂. The reaction was quenched with sodium sulfate decahydrate. The mixture was filtered and the filtrate was concentrated. The residue was purified by flash column chromatography to afford (2,6-dimethylenetetrahydro-1H-pyrrolizin-7a(5H)-yl)methanol (1.4 g, 81.9% yield) as a colorless oil.

¹H NMR (400 MHz, CDCl₃) δ 4.99-4.89 (m, 4H), 3.70 (d, J=14.9 Hz, 2H), 3.33-3.22 (m, 4H), 2.99 (s, 1H), 2.55 (d, J=16.4 Hz, 2H), 2.40 (d, J=16.4 Hz, 2H).

### Intermediate 4: (S)-(2-methylenetetrahydro-1H-pyrrolizin-7a(5H)-yl)methanol

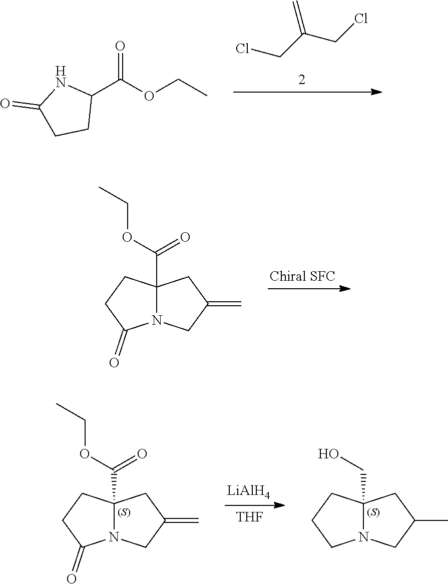

### Step 1. ethyl 2-methylene-5-oxotetrahydro-1H-pyrrolizine-7a(5H)-carboxylate

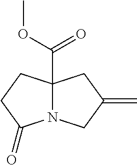

To a mixture of ethyl 5-oxopyrrolidine-2-carboxylate (20 g, 0.13 mol) and 3-chloro-2-(chloromethyl)prop-1-ene (32.34 g, 0.21 mol) in THF (200 mL) was added LiHMDS (1 M, 260 mL, 0.26 mol) drop-wise at −40° C. under N₂. The mixture was stirred at 30° C. overnight. The reaction mixture was poured into HCl (1 M, 250 mL) and pH was adjusted to 7 with HCl (2 M) at 0° C. The mixture was extracted with EA twice. The combined organic layers were washed with brine, dried over Na₂SO₄, filtered and concentrated under reduced pressure to give a residue. The residue was purified by column chromatography (Petroleum ether/Ethyl acetate=1:1) to afford the title compound ethyl 2-methylene-5-oxotetrahydro-1H-pyrrolizine-7a(5H)-carboxylate (12 g, 45.1% yield) as a yellow oil.

MS (ESI) m/z: 210 [M+H]⁺.

### Step 2. ethyl (S)-2-methylene-5-oxotetrahydro-1H-pyrrolizine-7a(5H)-carboxylate

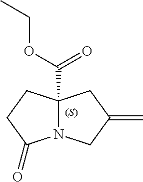

Ethyl 2-methylene-5-oxotetrahydro-1H-pyrrolizine-7a(5H)-carboxylate (5.2 g) was separated by Chiral SFC (Column: CHIRALPAK 1H, 50*250 mm; Mobile Phase A: CO2, Mobile Phase B: EtOH; Flow rate: 150 mL/min; Gradient: 26% B; 220 nm; RT1 (min): 1.094; RT2 (min): 1.417; Injection Volume: 1.8 mL Number Of Runs: 122) to give ethyl (R)-2-methylene-5-oxotetrahydro-1H-pyrrolizine-7a(5H)-carboxylate (2.29 g, the faster peak) and ethyl (S)-2-methylene-5-oxotetrahydro-1H-pyrrolizine-7a(5H)-carboxylate (2.12 g, the slower peak).

MS (ESI) m/z: 210 [M+H]⁺.

### Step 3. (S)-(2-methylenetetrahydro-1H-pyrrolizin-7a(5H)-yl)methanol

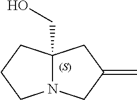

To a solution of ethyl (S)-2-methylene-5-oxotetrahydro-1H-pyrrolizine-7a(5H)-carboxylate (1.0 g, 4.78 mmol) in THF (30 mL) was added LiAlH₄ (4.78 mL, 2M in THF, 9.56 mmol) at 0° C. The mixture was stirred at 70° C. for 30 mins. The mixture was quenched with Na₂SO₄-10H₂O at room temperature and filtered. The mixture was concentrated under vacuum to provide the title compound (620 mg, 84.7% yield).

MS (ESI) m/z: 154 [M+H]⁺.

### Intermediate 5: tert-butyl (8aS,9S,12R)-5-chloro-4-fluoro-2-oxo-2,3,8a,9,10,11,12,13-octahydro-8H-7-oxa-1,3,6,13a,14-pentaaza-9,12-methanonaphtho[1,8-ab]heptalene-14-carboxylate

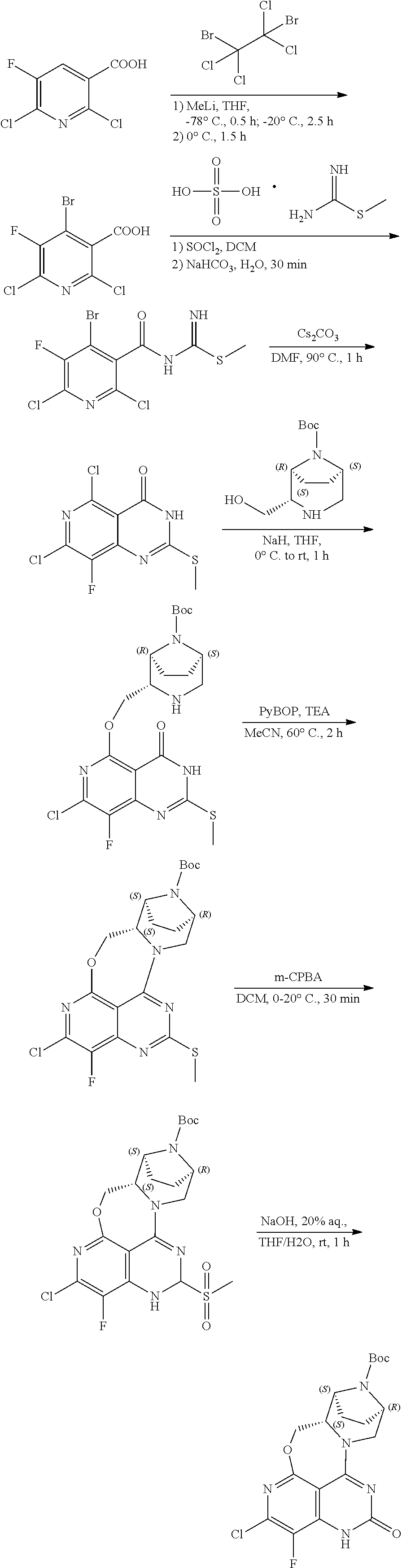

### Step 1: 4-bromo-2,6-dichloro-5-fluoronicotinic acid

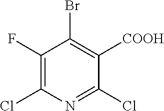 [SMILES: O=C(O)c1c(Cl)nc(Cl)c(F)c1Br]

To a cold solution of 2,6-dichloro-5-fluoronicotinic acid (50 g, 239.4 mmol) in anhydrous THF (500 mL) was added MeLi (2 mol/L, 240 mL) at −78° C. over 30 min, then the reaction was warmed to −20~−30° C. and stirred for 2 hours. The reaction mixture was cooled to −78° C. Followed by adding 1,2-dibromo-1,1,2,2-tetrachloroethane (85 g, 261.5 mmol) in anhydrous THF (300 mL). Then the reaction mixture was stirred at 0° C. for 1.5 hours. The reaction solution was diluted with ice water (500 mL) and the reaction was extracted with chloroform (800 mL), the water layer was separated and adjusted pH=2 by adding 1M hydrochloric acid in water and extracted by Ethyl acetate (500 mL×3). The organic layer was combined and dried over Na₂SO₄, filtered and concentrated to give the title product (38 g, 55.35%) as a white solid used to the next step directly without further purification.

LCMS: [2M+H]⁺=576.7

### Step 2: methyl (4-bromo-2,6-dichloro-5-fluoronicotinoyl) carbamimidothioate

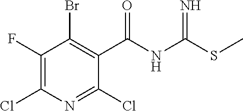 [SMILES: CSC(=N)NC(=O)c1c(Cl)nc(Cl)c(F)c1Br]

16 drops of DMF was added to a solution of 4-bromo-2,6-dichloro-5-fluoronicotinic acid (38 g, 132.4 mmol) in SOCl₂ (240 mL) at room temperature. The reaction was stirred at 100° C. for 1 hour, the solvent was concentrated under reduced pressure. The crude product was added to the mixture of THF (400 mL) at 0° C. and saturated sodium bicarbonate (400 mL). Methyl carbamimidothioate sulfuric acid (122 g, 438.8 mmol) was added, the reaction was stirred at room temperature for 30 minutes. The reaction was extracted with EA (500 ml×3). The organic layer was washed with brine (800 ml), dried over anhydrous sodium sulfate, filtered and concentrated to afford the title product (44 g, yield: 92.6%) as a yellow solid.

LCMS: [M−H]⁺: 359.9

### Step 3: 5,7-dichloro-8-fluoro-2-(methylthio)pyrido[4,3-d]pyrimidin-4(3H)-one

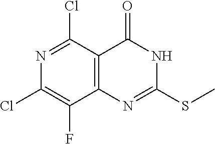 [SMILES: CSc1nc2c(F)c(Cl)nc(Cl)c2c(=O)[nH]1]

To a solution of methyl(4-bromo-2,6-dichloro-5-fluoronicotinoyl) carbamimidothioate (44 g, 122 mmol) in DMF (450 mL) was added Cs₂CO₃ (60 g, 183 mmol), and the reaction stirred at 90° C. for 1 hour. The reaction was cooled to room temperature and poured into ice water (300 mL). The pH of aqueous phase was adjusted to 2-3, filtered, and washed the filtered cake with water. The filtered cake was dried in vacuum dryer to give the title product (28 g, yield: 82.1%) as a white solid.

LCMS: [M+H]⁺: 280

### Step 4. tert-butyl (1R,2S,5S)-2-(((7-chloro-8-fluoro-2-(methylthio)-4-oxo-3,4-dihydropyrido[4,3-d]pyrimidin-5-yl)oxy)methyl)-3,8-diazabicyclo[3.2.1]octane-8-carboxylate

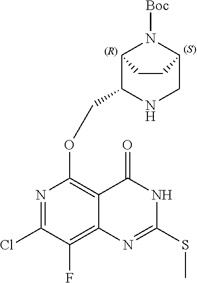

To a solution of tert-butyl (1R,2S,5S)-2-(hydroxymethyl)-3,8-diazabicyclo[3.2.1]octane-8-carboxylate (500 mg, 2.06 mmol) in THF (10 mL) was added NaH (225 mg, 60% in oil, 5.62 mmol) at 0° C. The mixture was stirred at room temperature for 20 minutes. 5,7-dichloro-8-fluoro-2-(methylthio)pyrido[4,3-d]pyrimidin-4(3H)-one (520 mg, 2.06 mmol) was added. The resulted mixture was stirred at room temperature for 1 hour. The reaction was quenched with saturated NH₄Cl solution. The aqueous layer was extracted with EA (200 mL×3), and the combined organic layers were washed with brine (50 mL×2), dried over anhydrous Na₂SO₄. After filtration, the filtrate was concentrated and the residue purified by silica gel column (MeOH:DCM=1:10) to give the product (600 mg, yield: 59%) as a yellow solid.

LCMS: [M+H]⁺=486.2.

### Step 5. tert-butyl (5aS,6S,9R)-2-chloro-1-fluoro-12-(methylthio)-5a,6,7,8,9,10-hexahydro-5H-4-oxa-3,10a,11,13,14-pentaaza-6,9-methanonaphtho[1,8-ab]heptalene-14-carboxylate

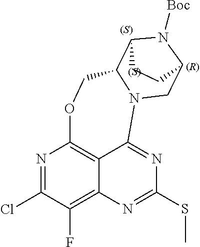

To a solution of tert-butyl (1R,2S,5S)-2-(((7-chloro-8-fluoro-2-(methylthio)-4-oxo-3,4-dihydropyrido[4,3-d]pyrimidin-5-yl)oxy)methyl)-3,8-diazabicyclo[3.2.1]octane-8-carboxylate (600 mg, 1.24 mmol) in MeCN (20 mL) were added PyBOP (1.30 g, 2.48 mmol) and TEA (375 mg, 3.36 mmol). The reaction mixture was stirred at 60° C. for 2 hours. The reaction was concentrated. The residue was purified by prep-TLC (DCM:MeOH=20:1) to afford the title product (500 mg, yield: 86%) as a yellow solid.

LCMS: [M+H]⁺=468.2.

### Step 6. tert-butyl (8aS,9S,12R)-5-chloro-4-fluoro-2-(methylsulfonyl)-2,3,8a,9,10,11,12,13-octahydro-8H-7-oxa-1,3,6,13a,14-pentaaza-9,12-methanonaphtho[1,8-ab]heptalene-14-carboxylate

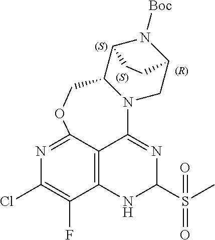

To a solution of tert-butyl (5aS,6S,9R)-2-chloro-1-fluoro-12-(methylthio)-5a,6,7,8,9,10-hexahydro-5H-4-oxa-3,10a,11,13,14-pentaaza-6,9-methanonaphtho[1,8-ab]heptalene-14-carboxylate (400 mg, 0.85 mmol) in DCM (10 mL) was added m-CPBA (295 mg, 1.72 mmol), and the reaction was stirred at room temperature for 30 minutes. The reaction was diluted with DCM and saturated NaHCO₃ solution. The organic layer was separated, washed with saturated NaHCO₃ solution, and concentrated in vacuo to afford the title compound (600 mg, crude) as a yellow solid.

LCMS: [M+H]⁺=501.2.

### Step 7. tert-butyl (8aS,9S,12R)-5-chloro-4-fluoro-2-oxo-2,3,8a,9,10,11,12,13-octahydro-8H-7-oxa-1,3,6,13a,14-pentaaza-9,12-methanonaphtho[1,8-ab]heptalene-14-carboxylate

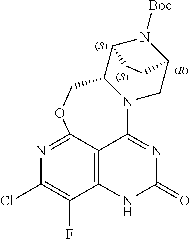

To a solution of tert-butyl (8aS,9S,12R)-5-chloro-4-fluoro-2-(methylsulfonyl)-2,3,8a,9,10,11,12,13-octahydro-8H-7-oxa-1,3,6,13a,14-pentaaza-9,12-methanonaphtho[1,8-ab]heptalene-14-carboxylate (600 mg, 1.19 mmol) in THF (10 mL) and water (5 mL) was added sodium hydroxide (96 mg. 2.40 mmol) at 0° C. and the reaction was stirred at room temperature for 1 h. The reaction was diluted with H₂O (20 mL) and extracted with EA (200 mL×3). The combined organic layers were washed with brine (50 mL×2), dried over anhydrous Na₂SO₄. After filtration, the filtrate was concentrated and the residue purified by silica gel column (MeOH:DCM=1:10) to give the title product (225 mg, yield: 62%).

LCMS: [M+H]⁺=438.2.

### Intermediate 6: tert-butyl (8S,8aS,9S,12R)-5-chloro-4-fluoro-8-methyl-2-oxo-2,3,8a,9,10,11,12,13-octahydro-8H-7-oxa-1,3,6,13a,14-pentaaza-9,12-methanonaphtho[1,8-ab]heptalene-14-carboxylate

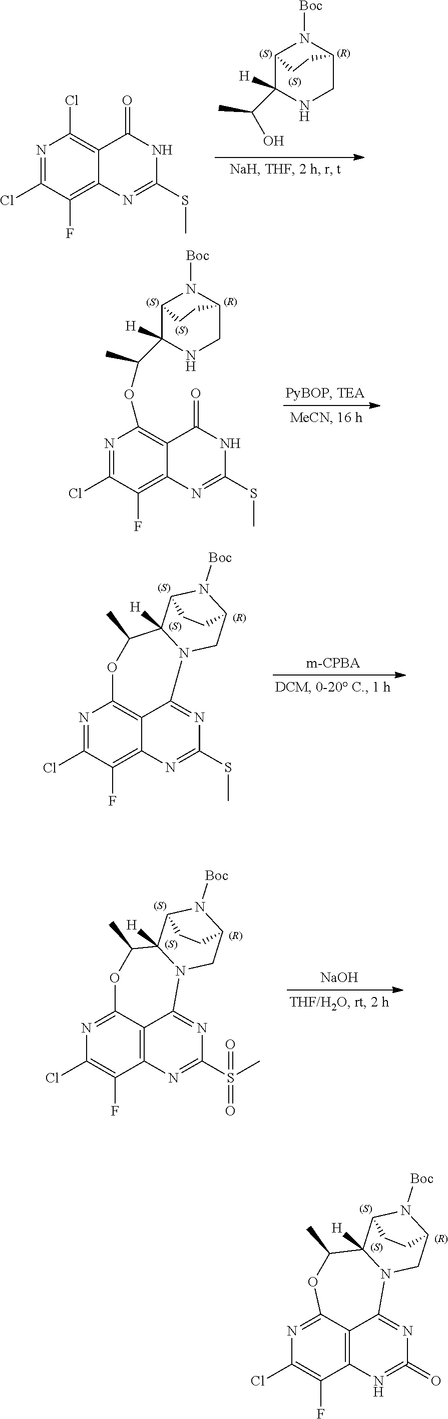

### Step 1. tert-butyl (1S,2S,5R)-2-((S)-1-((7-chloro-8-fluoro-2-(methylthio)-4-oxo-3,4-dihydropyrido[4,3-d]pyrimidin-5-yl)oxy)ethyl)-3,8-diazabicyclo[3.2.1]octane-8-carboxylate

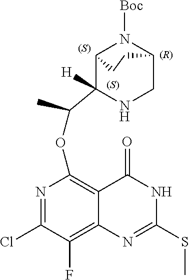

To a solution of tert-butyl (1S,2S,5R)-2-((S)-1-hydroxyethyl)-3,8-diazabicyclo[3.2.1]octane-8-carboxylate (3.51 g, 13.67 mol) in THF (60 mL) was added NaH (1.64 g, 41.02 mmol) at 0° C. under N₂ atmosphere, then stirred at 0° C. for 30 minutes before 5,7-dichloro-8-fluoro-2-(methylthio)pyrido[4,3-d]pyrimidin-4(3H)-one (3.1 g, 10.94 mmol) was added, the resulted mixture was stirred for 2 hours at 0° C. to room temperature under N₂ atmosphere. The mixture was diluted with water (50 mL), extracted with EtOAc (50 mL×3), the combined organic layers were dried over Na₂SO₄, filtered, and concentrated in vacuo, the residue was purified by silica column using DCM:MeOH (30:1) as eluent to afford product (2.3 g crude) as a yellow oil, used for next step without further purification.

LCMS: (M+H)⁺=500.2

### Step 2: tert-butyl (5S,5aS,6S,9R)-2-chloro-1-fluoro-5-methyl-12-(methylthio)-5a,6,7,8,9,10-hexahydro-5H-4-oxa-3,10a,11,13,14-pentaaza-6,9-methanonaphtho[1,8-ab]heptalene-14-carboxylate

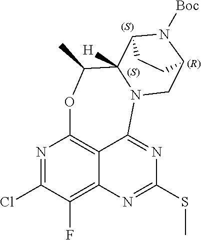

To a solution of tert-butyl (1S,2S,5R)-2-((S)-1-((7-chloro-8-fluoro-2-(methylthio)-4-oxo-3,4-dihydro pyrido[4,3-d]pyrimidin-5-yl)oxy)ethyl)-3,8-diazabicyclo[3.2.1]octane-8-carboxylate (2.3 g crude) in MeCN (40 mL) was added PyBOP (2.48 g, 4.80 mmol) and TEA (1.2 g, 12.00 mmol) at room temperature under N₂ atmosphere, then stirred at 80° C. for 16 hours. The mixture was quenched with sat. NaHCO₃ (50 mL), extracted with EtOAc (20 mL×3), the combined organic layers were dried over Na₂SO₄, filtered, and concentrated in vacuo, the residue was purified by silica column using EA:PE (1:3) as eluent to afford the product (1.3 g, yield: 38%, 2 steps) as a yellow solid.

LCMS: (M+H)⁺=482.1

### Step 3: tert-butyl (8S,8aS,9S,12R)-5-chloro-4-fluoro-8-methyl-2-(methylsulfonyl)-8a,9,10,11,12,13-hexahydro-8H-7-oxa-1,3,6,13a,14-pentaaza-9,12-methanonaphtho[1,8-ab]heptalene-14-carboxylate

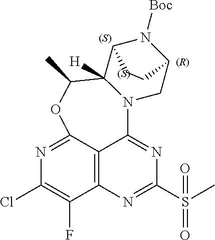

A mixture of (5S,5aS,6S,9R)-2-chloro-1-fluoro-5-methyl-12-(methylthio)-5a,6,7,8,9,10-hexahydro-5H-4-oxa-3,10a,11,13,14-pentaaza-6,9-methanonaphtho[1,8-ab]heptalene-14-carboxylate (1.31 g, 2.70 mmol) in DCM (30 mL) was added m-CPBA (900 mg, 5.405 mmol) at 0° C., and stirred for 1 hour at the same temperature, then the mixture was quenched with sat. NaHCO₃ (50 mL), extracted with EtOAc (50 mL×3), the combined organic layers were dried over Na₂SO₄, filtered, and concentrated in vacuo to afford the product (2.1 g, crude) as a yellow solid, which was used directly in the next step without further purification.

LCMS: (M+H)⁺=514.1

### Step 4: tert-butyl (8S,8aS,9S,12R)-5-chloro-4-fluoro-8-methyl-2-oxo-2,3,8a,9,10,11,12,13-octahydro-8H-7-oxa-1,3,6,13a,14-pentaaza-9,12-methanonaphtho[1,8-ab]heptalene-14-carboxylate

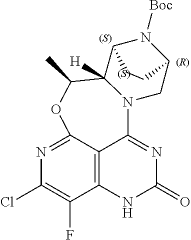

To a mixture of (8S,8aS,9S,12R)-5-chloro-4-fluoro-8-methyl-2-(methylsulfonyl)-8a,9,10,11,12,13-hexahydro-8H-7-oxa-1,3,6,13a,14-pentaaza-9,12-methanonaphtho[1,8-ab]heptalene-14-carboxylate (2.11 g, 4.09 mmol) in THF (10 mL) and H₂O (3 mL) was added NaOH (490 mg, 12.28 mmol) at room temperature, and stirred for 2 hours at the same temperature, then the mixture was diluted with water (50 mL), extracted with EtOAc (50 mL×3), the combined organic layers were dried over Na₂SO₄, filtered, and concentrated in vacuo, the residue was purified by silica column using EA:PE (5:1) as eluent to afford the product (810 mg, yield: 43%) as a yellow solid.

LCMS: (M+H)⁺=452.1

### Intermediate 7: tert-butyl (5aS,6S,9R)-2-bromo-1,3-difluoro-13-oxo-5a,6,7,8,9,10,13,14-octahydro-5H-6,9-epiminoazepino[2′,1′:3,4][1,4]oxazepino[5,6,7-de]quinazoline-15-carboxylate

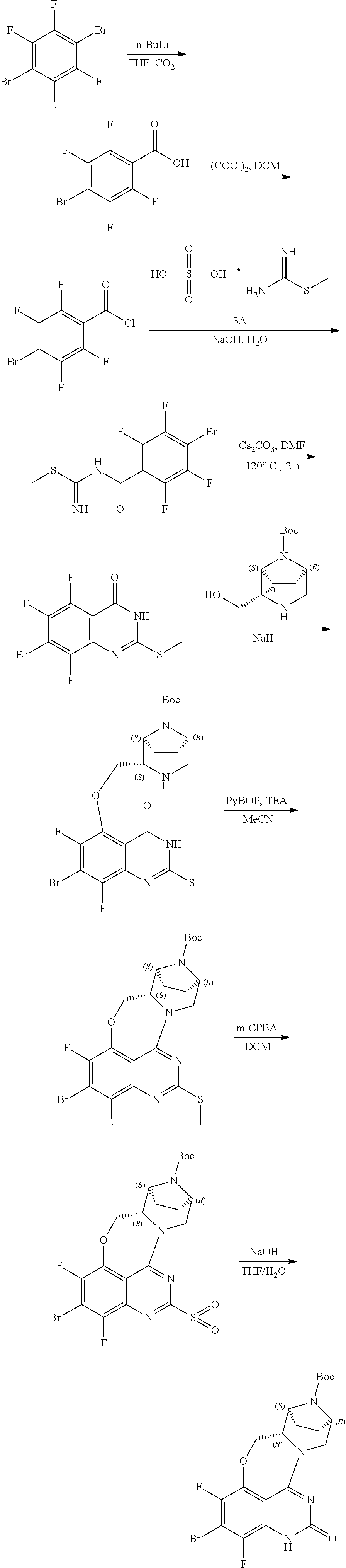

### Step 1. 4-bromo-2,3,5,6-tetrafluorobenzoic acid

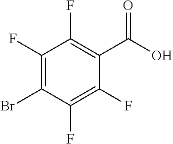 [SMILES: O=C(O)c1c(F)c(F)c(Br)c(F)c1F]

To a solution of 1,4-dibromo-2,3,5,6-tetrafluorobenzene (50 g, 162.4 mmol) in THF (500 mL) was added n-butyllithium (1.6 M solutions in hexanes, 106 mL, 169.6 mmol) dropwise at −78° C. under nitrogen atmosphere. Then the solution was stirred at the same temperature for 30 minutes, excess solid dry ice was added and the mixture was stirred at −78° C. for 1 h. The reaction mixture was slowly warmed to 0° C. and quenched with 2M HCL carefully. The solvent was removed on rotary evaporator and product was extracted with DCM (300 mL×3). The organic phase was washed with saturated sodium thiosulfate solution (300 mL), brine solution (300 mL) and the organic layer was combined and dried over Na₂SO₄, filtered and concentrated to give 4-bromo-2,3,5,6-tetrafluorobenzoic acid (36 g, 81.2%) as a white solid which used for the next step directly without further purification.

### Step 2. methyl (4-bromo-2,3,5,6-tetrafluorobenzoyl) carbamimidothioate

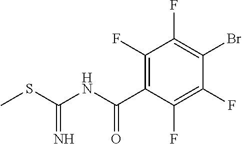 [SMILES: CSC(=N)NC(=O)c1c(F)c(F)c(Br)c(F)c1F]

To a solution of 4-bromo-2,3,5,6-tetrafluorobenzoic acid (30 g, 110 mmol) was dissolved in DCM (300 mL) at room temperature, followed by adding 16 drops of DMF. The reaction was cooled to 0° C. and was added Oxalyl chloride (56 g, 440 mmol). The reaction was stirred at 25° C. for 1 hour. The solvent was concentrated under reduced pressure. The crude product was added into a mixture of THF (400 mL) and NaOH/water (20.2 g, 900 ml) at 0° C., then methyl carbamimidothioate sulfuric acid (92 g, 330 mmol) was added, the reaction was stirred at room temperature for 30 minutes. The reaction was then extracted with EA (500 ml×3). The organic layer was washed with brine (800 ml), dried over anhydrous sodium sulfate, filtered and concentrated, methyl (4-bromo-2,3,5,6-tetrafluorobenzoyl)carbamimidothioate (35 g, yield: 92%) was obtained as a yellow solid.

LCMS: (M+H)⁺=344.9

### Step 3. 7-bromo-5,6,8-trifluoro-2-(methylthio) quinazolin-4(3H)-one

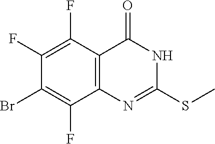 [SMILES: CSc1nc2c(F)c(Br)c(F)c(F)c2c(=O)[nH]1]

To a solution of methyl (4-bromo-2,3,5,6-tetrafluorobenzoyl) carbamimidothioate (35 g, 102 mmol) in DMF (350 mL) was added Cs₂CO₃ (49 g, 150 mmol), and the reaction stirred at 120° C. for 3 hours. The reaction was cooled to room temperature and poured into ice water (300 mL). The pH of aqueous phase was adjusted to 2-3 with 1M HCl, solid precipitated. After filtered, washed the filter residue with water. The residue was dried in vacuum to give the 7-bromo-5,6,8-trifluoro-2-(methylthio) quinazolin-4(3H)-one (20 g, yield: 60%) as a white solid.

LCMS: (M+H)⁺=326.9

### Step 4. tert-butyl (1S,2S,5R)-2-(((7-bromo-6,8-difluoro-2-(methylthio)-4-oxo-3,4-dihydroquinazolin-5-yl)oxy)methyl)-3,8-diazabicyclo[3.2.1]octane-8-carboxylate

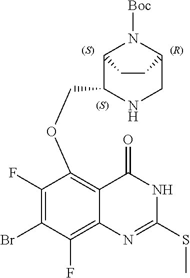

To a solution of tert-butyl (1S,2S,5R)-2-(hydroxymethyl)-3,8-diazabicyclo[3.2.1]octane-8-carboxylate (7 g, 28.9 mmol) in THF (100 mL) was added NaH (2.5 g, 60% in oil, 62.5 mmol) at 0° C. The mixture was stirred at room temperature for 20 minutes, before 7-bromo-5,6,8-trifluoro-2-(methylthio) quinazolin-4(3H)-one (8.4 g, 25.9 mmol) was added. The resulted mixture was continue stirred at room temperature for 1 hour. The reaction was quenched with saturated NH₄Cl solution. The aqueous layer was extracted with EA (200 mL×3), and the combined organic layers were washed with brine (50 mL×2), dried over anhydrous Na₂SO₄. After filtration, the filtrate was concentrated and the residue purified by silica gel column (MeOH:DCM=1:10) to give the product (7.5 g, yield: 52%) as a yellow solid.

LCMS: [M+H]⁺=549.4

### Step 5. tert-butyl (5aS,6S,9R)-2-bromo-1,3-difluoro-13-(methylthio)-5a,6,7,8,9,10-hexahydro-5H-6,9-epiminoazepino[2′,1′:3,4][1,4]oxazepino[5,6,7-de]quinazoline-15-carboxylate

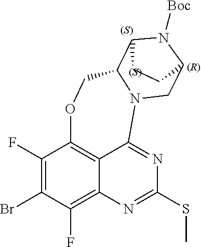

To a solution of tert-butyl (1S,2S,5R)-2-(((7-bromo-6,8-difluoro-2-(methylthio)-4-oxo-3,4-dihydroquinazolin-5-yl)oxy)methyl)-3,8-diazabicyclo[3.2.1]octane-8-carboxylate (7 g, 12.8 mmol) in MeCN (80 mL) were added PyBOP (13.4 g, 25.6 mmol) and TEA (3.9 g, 38.5 mmol), the reaction was stirred at 60° C. for 2 hours. The reaction was concentrated. The crude material was loaded on a silica gel plate. The plate was developed using DCM:MeOH=20:1 to afford the title compound (5.2 g, yield: 76%) as a yellow solid.

LCMS: [M+H]⁺=528.9

### Step 6. tert-butyl (5aS,6S,9R)-2-bromo-1,3-difluoro-13-(methylsulfonyl)-5a,6,7,8,9,10-hexahydro-5H-6,9-epiminoazepino[2′,1′:3,4][1,4]oxazepino[5,6,7-de]quinazoline-15-carboxylate

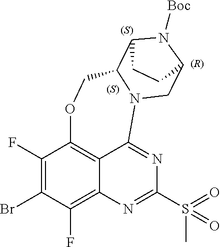

To a solution of tert-butyl (5aS,6S,9R)-2-bromo-1,3-difluoro-13-(methylthio)-5a,6,7,8,9,10-hexahydro-5H-6,9-epiminoazepino[2′,1′:3,4][1,4]oxazepino[5,6,7-de]quinazoline-15-carboxylate (5 g, 9.47 mmol) in DCM (80 mL) was added m-CPBA (4.9 g, 28.4 mmol), and the reaction was stirred at room temperature for 30 minutes. The reaction was diluted with DCM (100 mL) and saturated NaHCO₃ (120 mL) solution. The organic layer was separated, washed with saturated NaHCO₃ (100 mL) solution, and concentrated in vacuo to afford the title compound (4 g, crude) as a yellow solid.

LCMS: [M+H]⁺=562.9

### Step 7. tert-butyl (5aS,6S,9R)-2-bromo-1,3-difluoro-13-oxo-5a,6,7,8,9,10,13,14-octahydro-5H-6,9-epiminoazepino[2′,1′:3,4][1,4]oxazepino[5,6,7-de]quinazoline-15-carboxylate

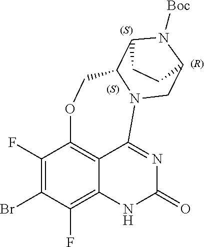

To a solution of tert-butyl (5aS,6S,9R)-2-bromo-1,3-difluoro-13-(methylsulfonyl)-5a,6,7,8,9,10-hexahydro-5H-6,9-epiminoazepino[2′,1′:3,4][1,4]oxazepino[5,6,7-de]quinazoline-15-carboxylate (4 g, 7.2 mmol) in THF (40 mL) and water (14 mL) was added sodium hydroxide (1.14 g. 28.6 mmol) at 0° C. and the reaction was stirred at room temperature for 16 hours. The reaction was diluted with H₂O (80 mL) and extracted with EA (100 mL×3). The combined organic layers were washed with brine (80 mL×2), dried over anhydrous Na₂SO₄. After filtration, the filtrate was concentrated and the residue purified by silica gel column (MeOH:DCM=1:10) to give the product (1.8 g, yield: 50%) as a yellow solid.

LCMS: [M+H]⁺=498.9

### Intermediate 8: tert-butyl (5aS,6S,9R)-2-bromo-3-chloro-1-fluoro-13-oxo-5a,6,7,8,9,10,13,14-octahydro-5H-6,9-epiminoazepino[2′,1′:3,4][1,4]oxazepino[5,6,7-de]quinazoline-15-carboxylate

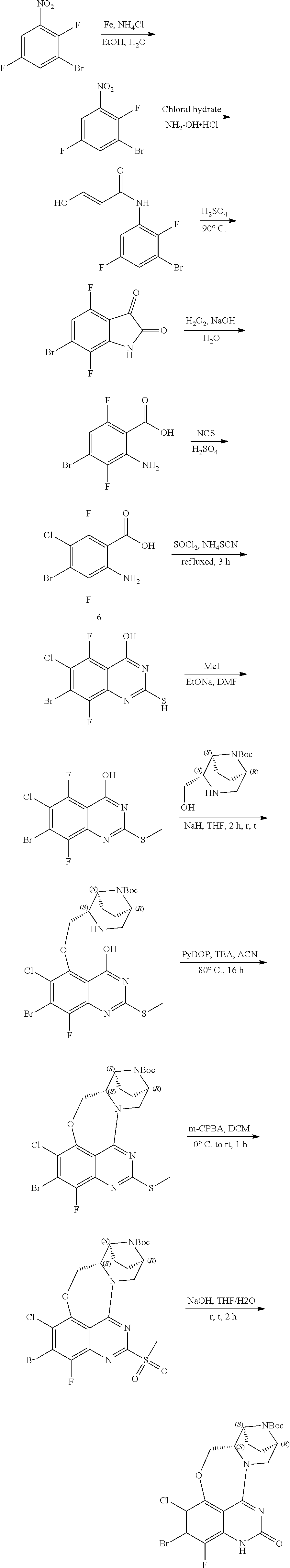

### Step 1: 3-bromo-2,5-difluoroaniline

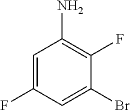

A solution of 1-bromo-2,5-difluoro-3-nitrobenzene (10.0 g, 42 mmol) in EtOH/water (100 mL/20 mL) was added Fe (11.8 g, 210 mmol) and NH₄Cl (11.3 g, 210 mmol), the reaction was stirred at 80° C. for 1 hour. The precipitated solids were filtered and washed with water, extracted with EtOAc (200 mL×2), the organics were dried over Na₂SO₄ and concentrated under vacuum to give crude product (8 g, yield: 93%), used in the next step directly without further purification.

LCMS: (M+H)⁺=209.0

### Step 2: N-(3-bromo-2,5-difluorophenyl)-3-hydroxyacrylamide

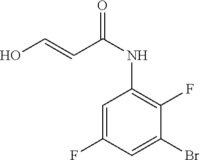 [SMILES: O=C(/C=C/O)Nc1cc(F)cc(Br)c1F]

A solution of chloral hydrate (9.0 g, 43.5 mmol) in water (180 mL) was added Na₂SO₄ (41.4 g, 174 mmol) and Hydroxylamine hydrochloride (9.1 g, 130.5 mmol), then another mixture of 3-bromo-2,5-difluoroaniline (9 g, 43.5 mmol) in water (18 mL)/EtOH (27 mL)/con. HCl (6.3 mL) was added, the resulted reaction was stirred at 60° C. for 18 hours. Cooled, the precipitated solids were collected by filtration and washed with water, dried under vacuum to give crude product (10 g, yield: 85%), used in the next step directly without further purification.

LCMS: (M−H)⁺=276.9

### Step 3: 6-bromo-4,7-difluoroindoline-2,3-dione

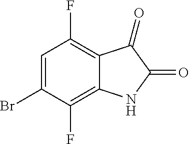 [SMILES: O=C1Nc2c(F)c(Br)cc(F)c2C1=O]

N-(3-bromo-2,5-difluorophenyl)-3-hydroxyacrylamide (10 g, 48.3 mmol) was added portion wise to H₂SO₄ (80 ml) at 60° C. The resulted reaction was stirred at 90° C. for 1 hour. The reaction mixture was cooled to room temperature and slowly added into ice water. The resulting precipitate was collected by filtration, washing with water and dried under vacuum to afford product (8.7 g, yield: 93%) as a dark red solid, which was used without further purification.

LCMS: (M−H)⁺=259.9

### Step 4: 2-amino-4-bromo-3,6-difluorobenzoic acid

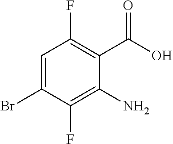 [SMILES: Nc1c(F)c(Br)cc(F)c1C(=O)O]

H₂O₂ (30% in H2O, 11 mL) was added dropwise to a solution of 6-bromo-4,7-difluoroindoline-2,3-dione (6.0 g, 28.91 mmol) in NaOH (2M in H₂O, 103 mL) at 0° C., then the reaction was stirred at ice bath for 2 hours. Excess H₂O₂ was quenched with excess Na₂SO₃, the solution was acidified to pH=2 with coc.HCl. The resulting cream precipitate was collected by filtration, washed with water and dried under vacuum to afford product (4.2 g, 73%) as a brown solid, which was used without further purification.

LCMS: (M−H)⁺=249.9

### Step 5: 2-amino-4-bromo-5-chloro-3,6-difluorobenzoic acid

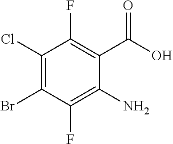 [SMILES: Nc1c(F)c(Br)c(Cl)c(F)c1C(=O)O]

NCS (4.2 g, 39 mmol) was added to a solution of 2-amino-4-bromo-3,6-difluorobenzoic acid (4 g, 19 mmol) in H₂SO₄ (40 mL) at room temperature and then the reaction mixture was stirred at 80° C. for 16 hours. The resulting solution was cooled to room temperature and poured onto ice. The precipitate was collected by filtration and dried under vacuum to afford product (3 g, 66%) as a yellow solid, which was used without further purification.

LCMS: (M−H)⁺=283.9

### Step 6: 7-bromo-6-chloro-5,8-difluoro-2-mercaptoquinazolin-4-ol

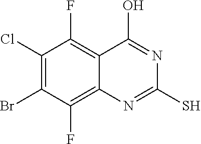 [SMILES: Oc1nc(S)nc2c(F)c(Br)c(Cl)c(F)c12]

A solution of 2-amino-4-bromo-5-chloro-3,6-difluorobenzoic acid (3 g, 10.5 mmol) in SOCl₂ (50 mL) was stirred at 50° C. for 3 hours. Concentrated and the residue was dissolved in acetone (15 mL), which was added into a solution of NH₄SCN (880 mg, 11.5 mmol) in acetone (40 mL) dropwise. The mixture was stirred at room temperature for 1 hour. The mixture was filtered and the filter cake was washed with acetone, the filtrate was concentrated under vacuum to afford product (3.2 g crude) as a brown solid, which was used in the next step directly without further purification.

LCMS: (M−H)⁺=324.9

### Step 7: 7-bromo-6-chloro-5,8-difluoro-2-(methylthio) quinazolin-4-ol

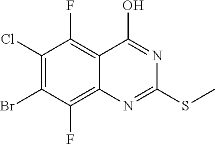 [SMILES: CSc1nc(O)c2c(F)c(Cl)c(Br)c(F)c2n1]

To a solution of 7-bromo-6-chloro-5,8-difluoro-2-mercaptoquinazolin-4-ol (3.2 g, 9.85 mmol) in DMF (40 mL) was added NaOEt (20 mL, 20 mmol) at 0° C., then Mel (1.56 g, 11 mmol) was added with stirred at 0° C. The reaction was stirred for 2 hours at the same temperature under N₂ atmosphere. The resulting mixture was poured into water (150 mL), extracted with EA (100 mL×3), the extracts were dried over Na₂SO₄, concentrated, the residue was purified by silica gel column (EA:PE=1:1 to 1:0) to afford desired product (2.5 g, yield: 78%) as a yellow solid

LCMS: (M+H)⁺=340.9

### Step 8: tert-butyl (1S,2S,5R)-2-(((7-bromo-6-chloro-8-fluoro-4-hydroxy-2-(methylthio)quinazolin-5-yl)oxy)methyl)-3,8-diazabicyclo[3.2.1]octane-8-carboxylate

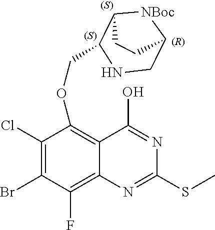

To a solution of tert-butyl (1S,2S,5R)-2-(hydroxymethyl)-3,8-diazabicyclo[3.2.1]octane-8-carboxylate (1.71 g, 7.02 mmol) in THF (50 mL) was added NaH (560 mg, 14.05 mmol) at 0° C., then stirred at 0° C. for 30 minutes under N₂ atmosphere before 7-bromo-6-chloro-5,8-difluoro-2-(methylthio) quinazolin-4-ol (2.15 g, 6.32 mmol) was added, the resulted mixture was stirred at 0° C. for 2 hours under N₂ atmosphere. The mixture was diluted with water (50 mL), extracted with EtOAc (50 mL×3), the combined organic layers were dried over Na₂SO₄, filtered, and concentrated in vacuo, the residue was purified by silica column using DCM:MeOH (30:1) as eluent to afford product (3 g crude) as a yellow oil, used for next step without further purification.

LCMS: (M+H)⁺=565.1

### Step 9: tert-butyl (5aS,6S,9R)-2-bromo-3-chloro-1-fluoro-13-(methylthio)-5a,6,7,8,9,10-hexahydro-5H-6,9-epiminoazepino[2′,1′:3,4][1,4]oxazepino[5,6,7-de]quinazoline-15-carboxylate

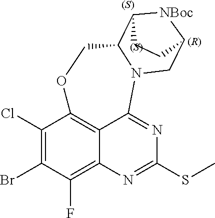

To a solution of tert-butyl (1S,2S,5R)-2-(((7-bromo-6-chloro-8-fluoro-4-hydroxy-2-(methylthio) quinazolin-5-yl)oxy)methyl)-3,8-diazabicyclo[3.2.1]octane-8-carboxylate (3 g crude) in MeCN (40 mL) was added PyBOP (5.52 g, 10.62 mmol) and TEA (1.6 g, 15.93 mmol) at room temperature, then stirred for 16 hours at 80° C. The mixture was quenched with sat. NaHCO₃ (100 mL), extracted with EtOAc (50 mL×3), the combined organic layers were dried over Na₂SO₄, filtered, and concentrated in vacuo, the residue was purified by silica column using EA:PE (1:3) as eluent to afford product (2.3 g, yield: 50%, 2 steps) as a yellow solid.

LCMS: (M+H)⁺=547.1

### Step 10: tert-butyl (5aS,6S,9R)-2-bromo-3-chloro-1-fluoro-13-(methylsulfonyl)-5a,6,7,8,9,10-hexahydro-5H-6,9-epiminoazepino[2′,1′:3,4][1,4]oxazepino[5,6,7-de]quinazoline-15-carboxylate

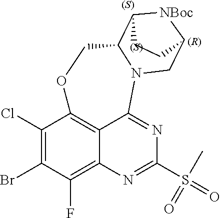

To a mixture of tert-butyl (5aS,6S,9R)-2-bromo-3-chloro-1-fluoro-13-(methylthio)-5a,6,7,8,9,10-hexahydro-5H-6,9-epiminoazepino[2′,1′:3,4][1,4]oxazepino[5,6,7-de]quinazoline-15-carboxylate (2.3 g, 4.21 mmol) in DCM (30 mL) was added m-CPBA (2.18 g, 12.63 mmol) at 0° C., and stirred for 1 hour at the same temperature, then the mixture was quenched with sat. NaHCO₃ (50 mL), extracted with EtOAc (50 mL×3), the combined organic layers were dried over Na₂SO₄, filtered, and concentrated in vacuo to afford product (2.9 g, crude) as a yellow solid, which was used directly in the next step without further purification.

LCMS: (M+H)⁺=577.0

### Step 11: tert-butyl (5aS,6S,9R)-2-bromo-3-chloro-1-fluoro-13-oxo-5a,6,7,8,9,10,13,14-octahydro-5H-6,9-epiminoazepino[2′,1′:3,4][1,4]oxazepino[5,6,7-de]quinazoline-15-carboxylate

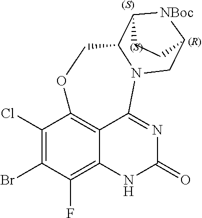

To a mixture of tert-butyl (5aS,6S,9R)-2-bromo-3-chloro-1-fluoro-13-(methylsulfonyl)-5a,6,7,8,9,10-hexahydro-5H-6,9-epiminoazepino[2′,1′:3,4][1,4]oxazepino[5,6,7-de]quinazoline-15-carboxylate (2.9 g, 5.03 mmol) in THF (20 mL) and H₂O (10 mL) was added NaOH (402 mg, 10.05 mmol) at room temperature, and stirred for 2 hour at the same temperature, then the mixture was diluted with water (50 mL), extracted with EtOAc (50 mL×3), the combined organic layers were dried over Na₂SO₄, filtered, and concentrated in vacuo, the residue was purified by silica column using EA:PE (5:1) as eluent to afford product (1.1 g, yield: 43%) as a yellow solid.

LCMS: (M+H)⁺=516.9

### Example 1

### (5aS,6S,9R)-2-(8-ethynyl-7-fluoronaphthalen-1-yl)-1-fluoro-13-(((2R,7aS)-2-fluorotetrahydro-1H-pyrrolizin-7a(5H)-yl)methyl)-5a,6,7,8,9,10-hexahydro-5H-4-oxa-3,10a,11,13,14-pentaaza-6,9-methanonaphtho[1,8-ab]heptalen-12(13H)-one

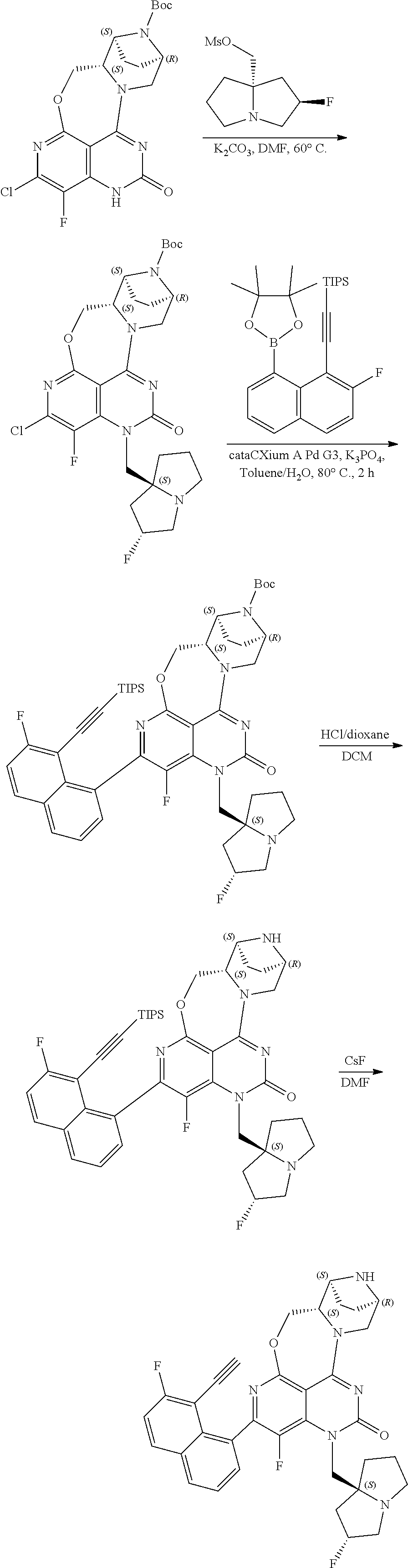

### Step 1: tert-butyl(8aS,9S,12R)-5-chloro-4-fluoro-3-(((2R,7aS)-2-fluorotetrahydro-1H-pyrrolizin-7a(5H)-yl)methyl)-2-oxo-2,3,8a,9,10,11,12,13-octahydro-8H-7-oxa-1,3,6,13a,14-pentaaza-9,12-methanonaphtho[1,8-ab]heptalene-14-carboxylate

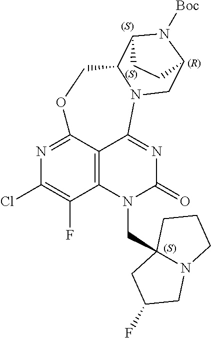

A mixture of tert-butyl (8aS,9S,12R)-5-chloro-4-fluoro-2-oxo-2,3,8a,9,10,11,12,13-octahydro-8H-7-oxa-1,3,6,13a,14-pentaaza-9,12-methanonaphtho[1,8-ab]heptalene-14-carboxylate (500 mg, 1.14 mmol), ((2R,7aS)-2-fluorotetrahydro-1H-pyrrolizin-7a(5H)-yl)methylmethanesulfonate (1.00 g, 4.22 mmol) and K₂CO₃ (315 mg, 2.28 mmol) in DMF (10 mL) was stirred for 16 hours at 60° C. under N₂ atmosphere. The reaction was diluted with H₂O (20 mL) and extracted with EA (30 mL×3). The combined organic layers were washed with brine (20 mL×2), dried over anhydrous Na₂SO₄. After filtration, the filtrate was concentrated and the residue purified by silica gel column (EA) to give the title product (180 mg, yield: 27%) as a yellow solid.

LCMS: (M+H)⁺=579.2

### Step 2: tert-butyl(5aS,6S,9R)-1-fluoro-2-(7-fluoro-8-((triisopropylsilyl)ethynyl)naphthalen-1-yl)-13-(((2R,7aS)-2-fluorotetrahydro-1H-pyrrolizin-7a(5H)-yl)methyl)-12-oxo-5a,6,7,8,9,10,12,13-octahydro-5H-4-oxa-3,10a,11,13,14-pentaaza-6,9-methanonaphtho[1,8-ab]heptalene-14-carboxylate

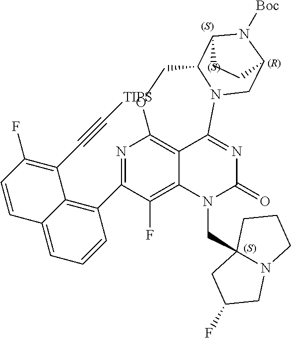

A mixture of tert-butyl(8aS,9S,12R)-5-chloro-4-fluoro-3-(((2R,7aS)-2-fluorotetrahydro-1H-pyrrolizin-7a(5H)-yl)methyl)-2-oxo-2,3,8a,9,10,11,12,13-octahydro-8H-7-oxa-1,3,6,13a,14-pentaaza-9,12-methanonaphtho[1,8-ab]heptalene-14-carboxylate (100 mg, 0.17 mmol), ((2-fluoro-8-(4,4,5,5-tetramethyl-1,3,2-dioxaborolan-2-yl)naphthalen-1-yl)ethynyl)triisopropylsilane (102 mg, 0.23 mmol), cataCXium A Pd G3 (13 mg, 0.02 mmol) and K₃PO₄ (110 mg, 0.52 mmol) in Toluene/H₂O (5 mL/1 mL) was stirred for 8 hours at 80° C. under N₂ atmosphere. The reaction was diluted with H₂O (20 mL) and extracted with EA (30 mL×3). The combined organic layers were washed with brine (20 mL×2), dried over anhydrous Na₂SO₄. After filtration, the filtrate was concentrated and the residue purified by silica gel column (EA) to give the title product (46 mg, yield: 30%) as a yellow solid.

LCMS: (M+H)⁺=869.2

### Step 3: (5aS,6S,9R)-1-fluoro-2-(7-fluoro-8-((triisopropylsilyl)ethynyl)naphthalen-1-yl)-13-(((2R,7aS)-2-fluorotetrahydro-1H-pyrrolizin-7a(5H)-yl)methyl)-5a,6,7,8,9,10-hexahydro-5H-4-oxa-3,10a,11,13,14-pentaaza-6,9-methanonaphtho[1,8-ab]heptalen-12(13H)-one

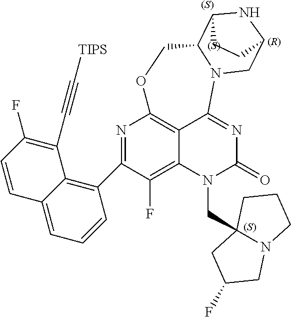

To a solution of tert-butyl(5aS,6S,9R)-1-fluoro-2-(7-fluoro-8-((triisopropylsilyl)ethynyl) naphthalen-1-yl)-13-(((2R,7aS)-2-fluorotetrahydro-1H-pyrrolizin-7a(5H)-yl)methyl)-12-oxo-5a,6,7,8,9,10,12,13-octahydro-5H-4-oxa-3,10a,11,13,14-pentaaza-6,9-methanonaphtho[1,8-ab]heptalene-14-carboxylate (40 mg, 0.05 mmol) in DCM (2 mL) was added 4M HCl/dioxane (2 mL). The reaction was stirred for 1 hour at 25° C. under N₂ atmosphere. The resulting mixture was concentrated under vacuum to give the title product (40 mg, crude) as a yellow oil, which was used for next step without further purification.

LCMS: (M+H)⁺=769.2

### Step 4: (5aS,6S,9R)-2-(8-ethynyl-7-fluoronaphthalen-1-yl)-1-fluoro-13-(((2R,7aS)-2-fluorotetrahydro-1H-pyrrolizin-7a(5H)-yl)methyl)-5a,6,7,8,9,10-hexahydro-5H-4-oxa-3,10a,11,13,14-pentaaza-6,9-methanonaphtho[1,8-ab]heptalen-12(13H)-one

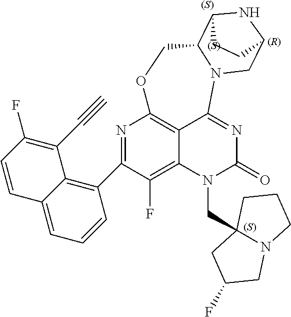

A mixture of (5aS,6S,9R)-1-fluoro-2-(7-fluoro-8-((triisopropylsilyl)ethynyl) naphthalen-1-yl)-13-(((2R,7aS)-2-fluorotetrahydro-1H-pyrrolizin-7a(5H)-yl)methyl)-5a,6,7,8,9,10-hexahydro-5H-4-oxa-3,10a,11,13,14-pentaaza-6,9-methanonaphtho[1,8-ab]heptalen-12(13H)-one (40 mg, 0.05 mmol) in DMF (1 mL) was added CsF (79 mg, 0.52 mmol) and the reaction was stirred for 1 hour at 25° C. under N₂ atmosphere. The resulting mixture was purified by pre-HPLC (0.05% NH₄HCO₃ in water/MeCN) to afford title product (2 mg, yield: 6%).

LCMS: (M+H)⁺=613.2

1H NMR (400 MHz, CD₃OD) δ 8.14-8.02 (m, 2H), 7.68-7.53 (m, 2H), 7.49-7.36 (m, 1H), 4.94 (s, 2H), 4.66-4.52 (m, 1H), 4.51-4.40 (m, 1H), 4.14-4.10 (m, 1H), 3.75-3.72 (m, 1H), 3.70-3.57 (m, 2H), 3.49-3.41 (m, 1H), 3.26-3.23 (m, 1H), 3.21-3.06 (m, 4H), 3.03-2.90 (m, 2H), 2.83-2.81 (m, 1H), 2.64-2.42 (m, 2H), 2.12-1.99 (m, 1H), 1.98-1.89 (m, 2H), 1.89-1.77 (m, 2H), 1.61-1.58 (m, 1H).

### Example 23

### (5aS,6S,9R)-2-(8-ethynyl-7-fluoro-3-hydroxynaphthalen-1-yl)-1-fluoro-13-(((2R,7aS)-2-fluorotetrahydro-1H-pyrrolizin-7a(5H)-yl)methyl)-5a,6,7,8,9,10-hexahydro-5H-4-oxa-3,10a,11,13,14-pentaaza-6,9-methanonaphtho[1,8-ab]heptalen-12(13H)-one

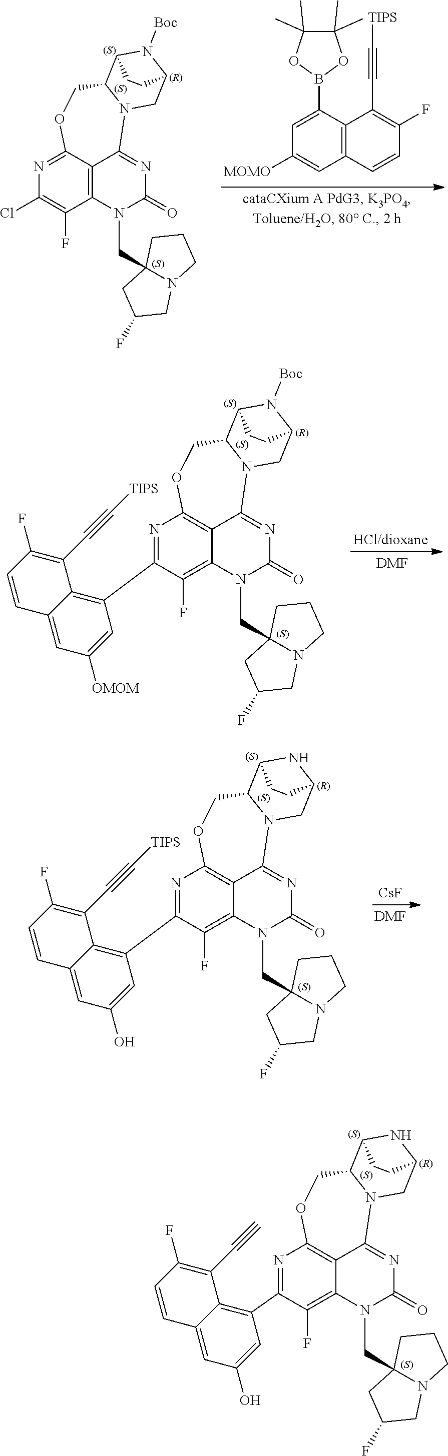

### Step 1: tert-butyl (5aS,6S,9R)-1-fluoro-2-(7-fluoro-3-(methoxymethoxy)-8-((triisopropylsilyl)ethynyl)naphthalen-1-yl)-13-(((2R,7aS)-2-fluorotetrahydro-1H-pyrrolizin-7a(5H)-yl)methyl)-12-oxo-5a,6,7,8,9,10,12,13-octahydro-5H-4-oxa-3,10a,11,13,14-pentaaza-6,9-methanonaphtho[1,8-ab]heptalene-14-carboxylate

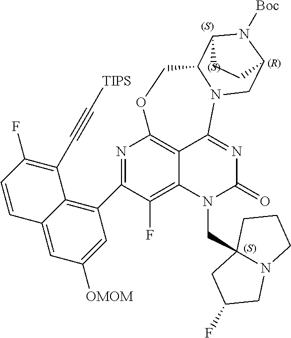

A solution of tert-butyl (8aS,9S,12R)-5-chloro-4-fluoro-3-(((2R,7aS)-2-fluorotetrahydro-1H-pyrrolizin-7a(5H)-yl)methyl)-2-oxo-2,3,8a,9,10,11,12,13-octahydro-8H-7-oxa-1,3,6,13a,14-pentaaza-9,12-methanonaphtho[1,8-ab]heptalene-14-carboxylate (80 mg, 0.138 mmol), ((2-fluoro-8-(4,4,5,5-tetramethyl-1,3,2-dioxaborolan-2-yl)naphthalen-1-yl)ethynyl)triisopropylsilane (82 mg, 0.23 mmol), cataCXium A Pd G3 (13 mg, 0.02 mmol) and K₃PO₄ (90 mg, 0.52 mmol) in Toluene/H₂O (5 mL/1 mL) was stirred for 8 hours at 80° C. under N₂ atmosphere. The reaction was diluted with H₂O (20 mL) and extracted with EA (50 mL×3). The combined organic layers were washed with brine (30 mL×2), dried over anhydrous Na₂SO₄. After filtration, the filtrate was concentrated and the residue purified by silica gel column to give the title product (26 mg, yield: 20%) as a yellow solid.

LCMS: (M+H)⁺=929.0

### Step 2: (5aS,6S,9R)-1-fluoro-2-(7-fluoro-3-hydroxy-8-((triisopropylsilyl)ethynyl)naphthalen-1-yl)-13-(((2R,7aS)-2-fluorotetrahydro-1H-pyrrolizin-7a(5H)-yl)methyl)-5a,6,7,8,9,10-hexahydro-5H-4-oxa-3,10a,11,13,14-pentaaza-6,9-methanonaphtho[1,8-ab]heptalen-12(13H)-one

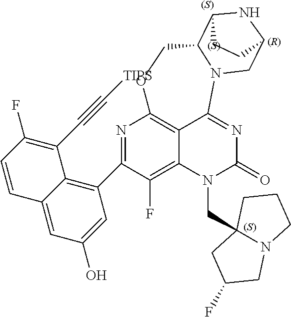

A solution of tert-butyl (5aS,6S,9R)-1-fluoro-2-(7-fluoro-3-(methoxymethoxy)-8-((triisopropylsilyl)-ethynyl)naphthalen-1-yl)-13-(((2R,7aS)-2-fluorotetrahydro-1H-pyrrolizin-7a(5H)-yl)methyl)-12-oxo-5a,6,7,8,9,10,12,13-octahydro-5H-4-oxa-3,10a,11,13,14-pentaaza-6,9-methanonaphtho[1,8-ab]heptalene-14-carboxylate (26 mg, 0.03 mmol) in MeCN (2 mL) was added 4M HCl/dioxane (2 mL). The reaction was stirred for 1 hour at 25° C. under N₂ atmosphere. The resulting mixture was concentrated under vacuum to give the title product (22 mg, crude) as a yellow oil, which was used for next step directly without further purification.

LCMS: (M+H)⁺=785.0

### Step 3: (5aS,6S,9R)-2-(8-ethynyl-7-fluoro-3-hydroxynaphthalen-1-yl)-1-fluoro-13-(((2R,7aS)-2-fluorotetrahydro-1H-pyrrolizin-7a(5H)-yl)methyl)-5a,6,7,8,9,10-hexahydro-5H-4-oxa-3,10a,11,13,14-pentaaza-6,9-methanonaphtho[1,8-ab]heptalen-12(13H)-one

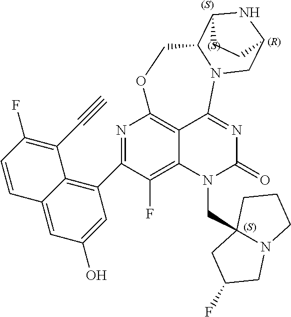

To a solution of (5aS,6S,9R)-1-fluoro-2-(7-fluoro-3-hydroxy-8-((triisopropylsilyl)ethynyl)naphthalen-1-yl)-13-(((2R,7aS)-2-fluorotetrahydro-1H-pyrrolizin-7a(5H)-yl)methyl)-5a,6,7,8,9,10-hexahydro-5H-4-oxa-3,10a,11,13,14-pentaaza-6,9-methanonaphtho[1,8-ab]heptalen-12(13H)-one (22 mg, 0.03 mmol) in DMF (1 mL) was added CsF (40 mg, 0.31 mmol) and the reaction was stirred for 1 hour at 25° C. under N₂ atmosphere. The resulting mixture was purified by pre-HPLC (0.05% NH₄HCO₃ in water/MeCN) to afford the title product (4 mg, yield: 6%).

LCMS: (M+H)⁺=628.9

¹H NMR (400 MHz, CD₃OD) δ 8.52 (s, 1H), 7.84 (d, J=6.2 Hz, 1H), 7.39-7.25 (m, 2H), 7.19 (d, J=20.6 Hz, 1H), 5.02-4.98 (m, 2H), 4.69-4.48 (m, 2H), 4.27-4.23 (m, 1H), 4.04-3.99 (m, 2H), 3.60-3.55 (m, 1H), 3.51-3.34 (m, 3H), 3.09-3.01 (m, 5H), 2.87-2.83 (m, 1H), 2.57-2.51 (m, 2H), 2.07-1.99 (m, 6H), 1.69-1.65 (m, 1H).

### Example 45

### (8aS,9S,12R)-5-(8-ethyl-7-fluoro-3-hydroxynaphthalen-1-yl)-4-fluoro-3-(((2R,7aS)-2-fluorotetrahydro-1H-pyrrolizin-7a(5H)-yl)methyl)-8a,9,10,11,12,13-hexahydro-8H-7-oxa-1,3,6,13a,14-pentaaza-9,12-methanonaphtho[1,8-ab]heptalen-2(3H)-one

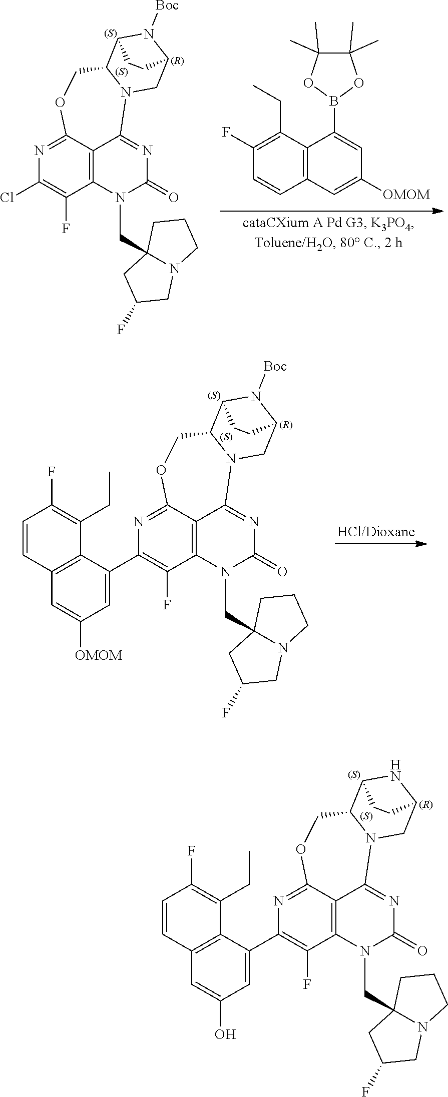

### Step 1. tert-butyl (8aS,9S,12R)-5-(8-ethyl-7-fluoro-3-(methoxymethoxy)naphthalen-1-yl)-4-fluoro-3-(((2R,7aS)-2-fluorotetrahydro-1H-pyrrolizin-7a(5H)-yl)methyl)-2-oxo-2,3,8a,9,10,11,12,13-octahydro-8H-7-oxa-1,3,6,13a,14-pentaaza-9,12-methanonaphtho[1,8-ab]heptalene-14-carboxylate

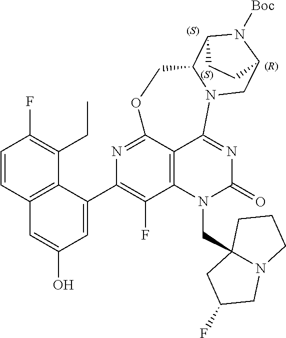

To a solution of the mixture products obtained from the previous reaction step (60 mg, 0.1 mmol) in toluene (3 mL) and H₂O (0.5 mL) were added 2-{8-ethyl-7-fluoro-3-[(methoxymethyl)oxy]-1-naphthyl}-4,4,5,5-tetramethyl-1,3,2-dioxaborolane (56 mg, 0.16 mmol), cataCXium A Pd G₃ (5.1 mg, 0.00 mmol) and K₃PO₄ (42 mg, 0.2 mmol), and the reaction was stirred at 80° C. for 2 hours under N₂. The mixture was extracted with EA (3×20 mL). Combined the EA layer and dried by Na₂SO₄, filtered and concentrated. The crude material was purified by column chromatography with DCM:MeOH=1:15 to afford a mixture products (35 mg) which was further purified by Pre-TLC (DCM:MeOH=1:15) to obtained the title product (8 mg, 10% yield).

MS (ESI) m/z: 777 [M+H]⁺.

### Step 2. (8aS,9S,12R)-5-(8-ethyl-7-fluoro-3-hydroxynaphthalen-1-yl)-4-fluoro-3-(((2R,7aS)-2-fluorotetrahydro-1H-pyrrolizin-7a(5H)-yl)methyl)-8a,9,10,11,12,13-hexahydro-8H-7-oxa-1,3,6,13a,14-pentaaza-9,12-methanonaphtho[1,8-ab]heptalen-2(3H)-one

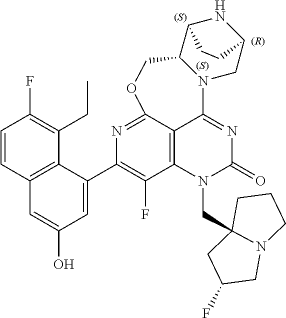

To a solution of tert-butyl (8aS,9S,12R)-5-(8-ethyl-7-fluoro-3-(methoxymethoxy)naphthalen-1-yl)-4-fluoro-3-(((2R,7aS)-2-fluorotetrahydro-1H-pyrrolizin-7a(5H)-yl)methyl)-2-oxo-2,3,8a,9,10,11,12,13-octahydro-8H-7-oxa-1,3,6,13a,14-pentaaza-9,12-methanonaphtho[1,8-ab]heptalene-14-carboxylate (8 mg, 0.01 mmol) in DCM (1 mL) were added HCl/Dioxane (1 mL; 4 M in dioxane), and the reaction was stirred at room temperature for 1 hour. The reaction was monitored by LCMS. When the reaction was completed, the reaction was concentrated to afford the residue. The residue was purified by prep-HPLC to give the title product (1.50 mg, 23.0% yield).

LC/MS ESI (m/z): 634 [M+H]⁺.

¹HNMR (400 MHz, CD₃OD) δ 7.65 (dd, J=8.9, 5.9 Hz, 1H), 7.31-7.20 (m, 2H), 7.04 (dd, J=33.7, 2.7 Hz, 1H), 5.04-4.95 (m, 1H), 4.82 (d, J=3.5 Hz, 2H), 4.65-4.52 (m, 3H), 4.22-4.15 (m, 1H), 3.93 (s, 1H), 3.84 (s, 1H), 3.23-3.12 (m, 4H), 3.00 (dd, J=12.7, 4.7 Hz, 2H), 2.86 (s, 1H), 2.60-2.43 (m, 3H), 2.14-1.83 (m, 7H), 0.90-0.81 (m, 3H).

### Example 178a and 178b

### (5aS,6S,9R)-2-(8-ethyl-7-fluoro-3-hydroxynaphthalen-1-yl)-1,3-difluoro-14-(((S)-2-methylenetetrahydro-1H-pyrrolizin-7a(5H)-yl)methyl)-5a,6,7,8,9,10-hexahydro-5H-6,9-epiminoazepino[2′,1′:3,4][1,4]oxazepino[5,6,7-de]quinazolin-13(14H)-one

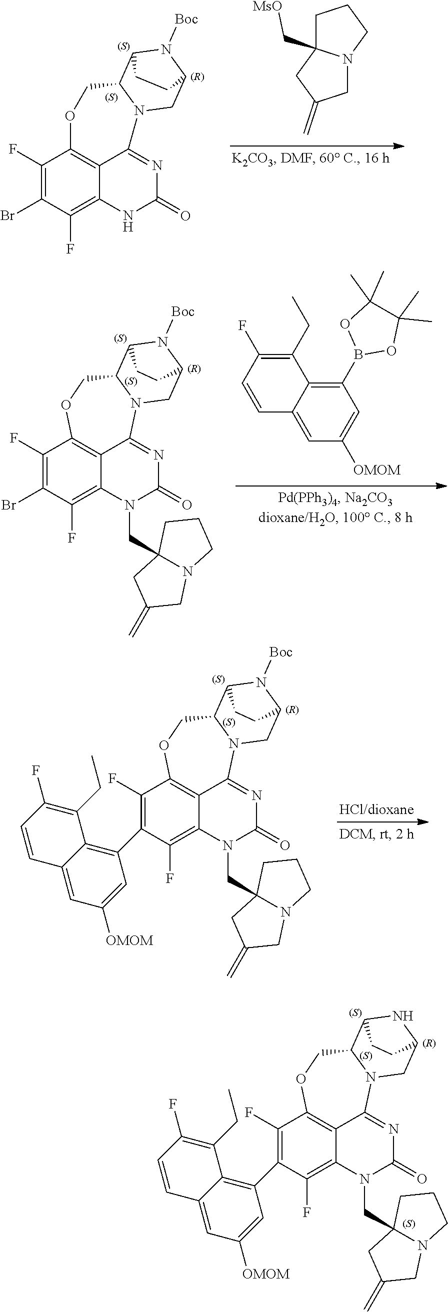

### Step 1: tert-butyl(5aS,6S,9R)-2-bromo-1,3-difluoro-14-(((S)-2-methylenetetrahydro-1H-pyrrolizin-7a(511)-yl)methyl)-13-oxo-5a,6,7,8,9,10,13,14-octahydro-5H-6,9-epiminoazepino[2′,1′:3,4][1,4]oxazepino[5,6,7-de]quinazoline-15-carboxylate

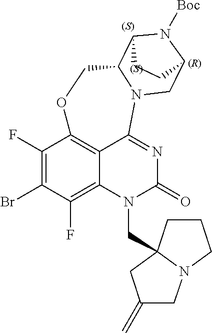

A solution of tert-butyl (5aS,6S,9R)-2-bromo-3-chloro-1-fluoro-13-oxo-5a,6,7,8,9,10,13,14-octahydro-5H-6,9-epiminoazepino[2′,1′:3,4][1,4]oxazepino[5,6,7-de]quinazoline-15-carboxylate (800 mg, 1.61 mmol), (S)-(2-methylenetetrahydro-1H-pyrrolizin-7a(5H)-yl)methyl methanesulfonate (1.0 g, 6.43 mmol) and K₂CO₃ (667 mg, 4.83 mmol) in DMF (15 mL) was stirred at 60° C. for 16 hours under N₂ atmosphere. The reaction was diluted with H₂O (20 mL) and extracted with EA (50 mL×3). The combined organic layers were washed with brine (50 mL×2), dried over anhydrous Na₂SO₄. After filtration, the filtrate was concentrated and the residue purified by silica gel column (MeOH:DCM=1:10) to give the Product (240 mg, yield: 23%) as a yellow solid.

LCMS: (M+H)⁺=634.2

### Step 2: tert-butyl(5aS,6S,9R)-2-(8-ethyl-7-fluoro-3-(methoxymethoxy)naphthalen-1-yl)-1,3-difluoro-14-(((S)-2-methylenetetrahydro-1H-pyrrolizin-7a(5H)-yl)methyl)-13-oxo-5a,6,7,8,9,10,13,14-octahydro-5H-6,9-epiminoazepino[2′,1′:3,4][1,4]oxazepino[5,6,7-de]quinazoline-15-carboxylate

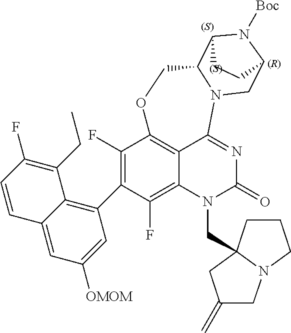

A mixture of tert-butyl (5aS,6S,9R)-2-bromo-1,3-difluoro-14-(((S)-2-methylenetetrahydro-1H-pyrrolizin-7a(5H)-yl)methyl)-13-oxo-5a,6,7,8,9,10,13,14-octahydro-5H-6,9-epiminoazepino[2′,1′:3,4][1,4]oxazepino[5,6,7-de]quinazoline-15-carboxylate (130 mg, 0.20 mmol), 2-(8-ethyl-7-fluoro-3-(methoxymethoxy)naphthalen-1-yl)-4,4,5,5-tetramethyl-1,3,2-dioxaborolane (144 mg, 0.40 mmol), Pd(PPh₃)₄ (46 mg, 0.04 mmol) and Na₂CO₃ (63 mg, 0.60 mmol) in dioxane/H₂O (5 mL/1 mL) was stirred at 100° C. for 8 hours under N₂ atmosphere. The reaction was diluted with H₂O (30 mL) and extracted with EA (30 mL×3). The combined organic layers were washed with brine (30 mL×2), dried over anhydrous Na₂SO₄. After filtration, the filtrate was concentrated and the residue purified by silica gel column (DCM:MeOH=10:1) to give the product (60 mg crude) as a brown solid, which was used for next step without further purification.

LCMS: (M+H)⁺=788.3

### Step 3: (5aS,6S,9R)-2-(8-ethyl-7-fluoro-3-hydroxynaphthalen-1-yl)-1,3-difluoro-14-(((S)-2-methylenetetrahydro-1H-pyrrolizin-7a(5H)-yl)methyl)-5a,6,7,8,9,10-hexahydro-5H-6,9-epiminoazepino[2′,1′:3,4][1,4]oxazepino[5,6,7-de]quinazolin-13(14H)-one

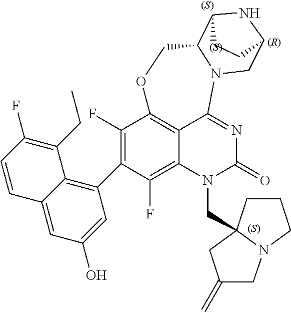

To a solution of tert-butyl (5aS,6S,9R)-2-(8-ethyl-7-fluoro-3-(methoxymethoxy)naphthalen-1-yl)-1,3-difluoro-14-(((S)-2-methylenetetrahydro-1H-pyrrolizin-7a(5H)-yl)methyl)-13-oxo-5a,6,7,8,9,10,13,14-octahydro-5H-6,9-epiminoazepino[2′,1′:3,4][1,4]oxazepino[5,6,7-de]quinazoline-15-carboxylate (50 mg, 0.06 mmol) in DCM (2 mL) was added HCl/dioxane (2 mL, 4M). The reaction was stirred at 25° C. for 2 hours. The resulting mixture was concentrated and purified by pre-HPLC (0.1% FA in water/MeCN) to afford title product (10 mg) as off-white solid. The stereoisomers were separated by chiral SFC (15 40% IPA DEA C2 IG) to afford isomer 1 (the first eluting isomer, 2 mg, yield: 5%) as a white solid and isomer 2 (the second eluting isomer, 4 mg, yield: 10%) as a white solid.

LCMS: (M+H)⁺=644.3

Isomer 1: Example 178A ¹H NMR: (400 MHz, CD₃OD) δ 7.68-7.64 (m, 1H), 7.30-7.20 (m, 2H), 7.00-6.96 (m, 1H), 5.01-4.95 (m, 1H), 4.92-4.89 (m, 1H), 4.83-4.81 (m, 1H), 4.62-4.54 (m, 1H), 4.43-4.33 (m, 1H), 4.17-4.11 (m, 1H), 3.78-3.71 (m, 1H), 3.70-3.57 (m, 2H), 3.56-3.47 (m, 2H), 3.46-3.40 (m, 1H), 3.37-3.33 (m, 1H), 3.29-3.24 (m, 1H), 3.14-2.93 (m, 3H), 2.84-2.75 (m, 1H), 2.57-2.37 (m, 3H), 2.22-2.08 (m, 1H), 2.00-1.88 (m, 3H), 1.86-1.76 (m, 1H), 1.60-1.49 (m, 1H), 0.81 (t, J=7.4 Hz, 3H).

Isomer 2: Example 178B ¹H NMR: (400 MHz, CD₃OD) δ 7.71-7.66 (m, 1H), 7.31-7.22 (m, 2H), 7.01-6.96 (m, 1H), 5.25-5.19 (m, 2H), 5.07-5.01 (m, 1H), 4.74-4.57 (m, 2H), 4.40-4.24 (m, 3H), 4.21-4.00 (m, 2H), 3.98-3.81 (m, 2H), 3.70-3.60 (m, 1H), 3.58-3.50 (m, 2H), 3.48-3.41 (m, 1H), 3.22-3.10 (m, 1H), 2.93-2.81 (m, 1H), 2.59-2.44 (m, 3H), 2.41-2.26 (m, 2H), 2.19-2.08 (m, 3H), 1.96-1.85 (m, 1H), 0.84 (t, J=7.3 Hz, 3H).

### Example 199

### (5S,5aS,6S,9R)-2-(8-ethynyl-7-fluoronaphthalen-1-yl)-1-fluoro-13-(((2R,7aS)-2-fluorotetrahydro-1H-pyrrolizin-7a(5H)-yl)methyl)-5-methyl-5a,6,7,8,9,10-hexahydro-5H-4-oxa-3,10a,11,13,14-pentaaza-6,9-methanonaphtho[1,8-ab]heptalen-12(13H)-one

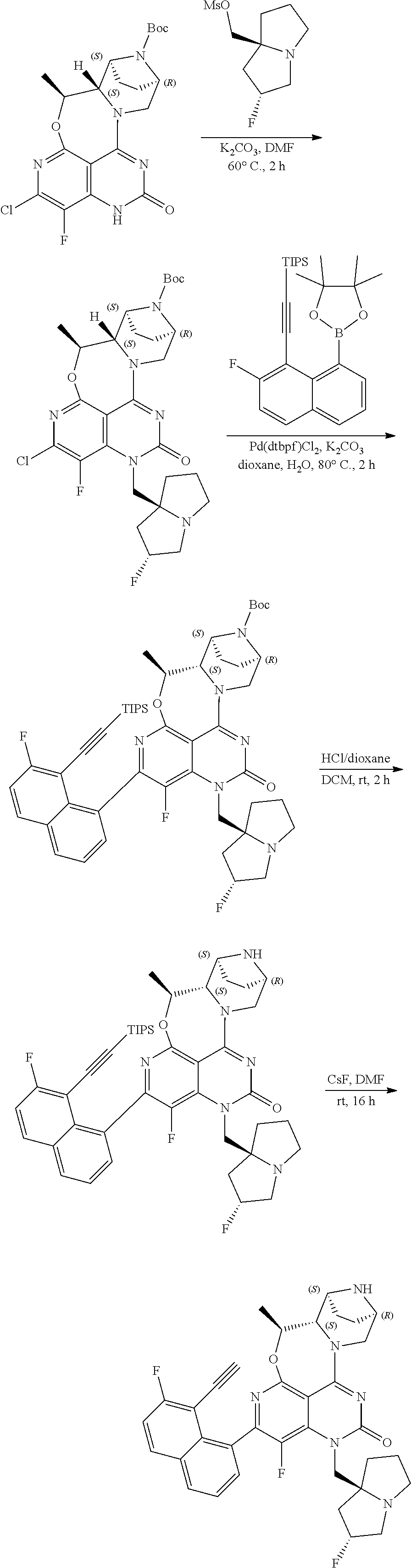

### Step 1: tert-butyl(8S,8aS,9S,12R)-5-chloro-4-fluoro-3-(((2R,7aS)-2-fluorotetrahydro-1H-pyrrolizin-7a(5H)-yl)methyl)-8-methyl-2-oxo-2,3,8a,9,10,11,12,13-octahydro-8H-7-oxa-1,3,6,13a,14-pentaaza-9,12-methanonaphtho[1,8-ab]heptalene-14-carboxylate

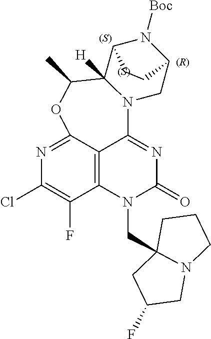

A mixture of tert-butyl (8S,8aS,9S,12R)-5-chloro-4-fluoro-8-methyl-2-oxo-2,3,8a,9,10,11,12,13-octahydro-8H-7-oxa-1,3,6,13a,14-pentaaza-9,12-methanonaphtho[1,8-ab]heptalene-14-carboxylate (450 mg, 1.00 mmol), ((2R,7aS)-2-fluorotetrahydro-1H-pyrrolizin-7a(5H)-yl)methyl methanesulfonate (948 g, 4.00 mmol) and K₂CO₃ (414 mg, 3.00 mmol) in DMF (10 mL) was stirred at 60° C. for 2 hours under N2 atmosphere. The reaction was diluted with H₂O (40 mL) and extracted with EA (50 mL×3). The combined organic layers were washed with brine (50 mL×2), dried over anhydrous Na₂SO₄. After filtration, the filtrate was concentrated and the residue purified by silica gel column (DCM:MeOH=10:1) to give the Product (160 mg, 27%) as a yellow solid.

LCMS: (M+H)⁺=593.2

### Step 2: tert-butyl(5S,5aS,6S,9R)-1-fluoro-2-(7-fluoro-8-((triisopropylsilyl)ethynyl)naphthalen-1-yl)-13-(((2R,7aS)-2-fluorotetrahydro-1H-pyrrolizin-7a(5H)-yl)methyl)-5-methyl-12-oxo-5a,6,7,8,9,10,12,13-octahydro-5H-4-oxa-3,10a,11,13,14-pentaaza-6,9-methanonaphtho[1,8-ab]heptalene-14-carboxylate

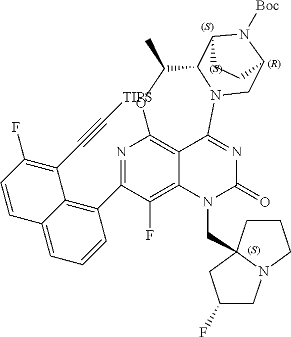

A mixture of tert-butyl (8S,8aS,9S,12R)-5-chloro-4-fluoro-3-(((2R,7aS)-2-fluorotetrahydro-1H-pyrrolizin-7a(5H)-yl)methyl)-8-methyl-2-oxo-2,3,8a,9,10,11,12,13-octahydro-8H-7-oxa-1,3,6,13a,14-pentaaza-9,12-methanonaphtho[1,8-ab]heptalene-14-carboxylate (80 mg, 0.13 mmol), ((2-fluoro-8-(4,4,5,5-tetramethyl-1,3,2-dioxaborolan-2-yl)naphthalen-1-yl)ethynyl)triisopropylsilane (117 mg, 0.26 mmol), Pd(dtbpf)Cl₂ (7 mg, 0.01 mmol) and K₂CO₃ (54 mg, 0.39 mmol) in dioxane/H₂O (5 mL/1 mL) was stirred at 80° C. for 2 hours under N₂ atmosphere. The reaction was diluted with H₂O (30 mL) and extracted with EA (30 mL×3). The combined organic layers were washed with brine (30 mL×2), dried over anhydrous Na₂SO₄. After filtration, the filtrate was concentrated and the residue purified by silica gel column (DCM:MeOH=10:1) to give the product (50 mg, purity: 52%) as a brown solid, which was used for next step without further purification.

LCMS: (M+H)⁺=883.5

### Step 3: (5S,5aS,6S,9R)-1-fluoro-2-(7-fluoro-8-((triisopropylsilyl)ethynyl)naphthalen-1-yl)-13-(((2R,7aS)-2-fluorotetrahydro-1H-pyrrolizin-7a(5H)-yl)methyl)-5-methyl-5a,6,7,8,9,10-hexahydro-5H-4-oxa-3,10a,11,13,14-pentaaza-6,9-methanonaphtho[1,8-ab]heptalen-12(13H)-one

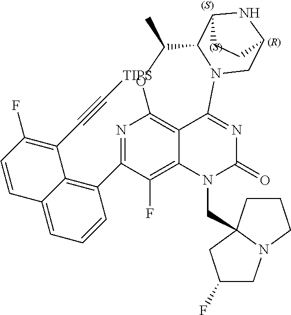

To a solution of tert-butyl (5S,5aS,6S,9R)-1-fluoro-2-(7-fluoro-8-((triisopropylsilyl)ethynyl) naphthalen-1-yl)-13-(((2R,7aS)-2-fluorotetrahydro-1H-pyrrolizin-7a(5H)-yl)methyl)-5-methyl-12-oxo-5a,6,7,8,9,10,12,13-octahydro-5H-4-oxa-3,10a,11,13,14-pentaaza-6,9-methanonaphtho[1,8-ab]heptalene-14-carboxylate (40 mg, 0.04 mmol) in DCM (2 mL) was added HCl/dioxane (2 mL, 4M). The reaction was stirred at 25° C. for 2 hours. The resulting mixture was concentrated to afford product (100 mg, crude) as a yellow solid, which was used in the next step directly without further purification.

LCMS: (M+H)⁺=783.5

### Step 4: (5S,5aS,6S,9R)-2-(8-ethynyl-7-fluoronaphthalen-1-yl)-1-fluoro-13-(((2R,7aS)-2-fluorotetrahydro-1H-pyrrolizin-7a(5H)-yl)methyl)-5-methyl-5a,6,7,8,9,10-hexahydro-5H-4-oxa-3,10a,11,13,14-pentaaza-6,9-methanonaphtho[1,8-ab]heptalen-12(13H)-one

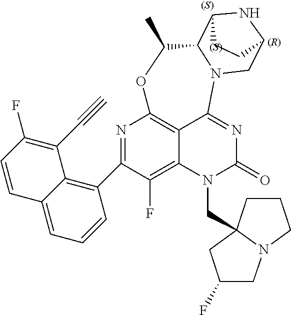

A mixture of (5S,5aS,6S,9R)-1-fluoro-2-(7-fluoro-8-((triisopropylsilyl)ethynyl)naphthalen-1-yl)-13-(((2R,7aS)-2-fluorotetrahydro-1H-pyrrolizin-7a(5H)-yl)methyl)-5-methyl-5a,6,7,8,9,10-hexahydro-5H-4-oxa-3,10a,11,13,14-pentaaza-6,9-methanonaphtho[1,8-ab]heptalen-12(13H)-one (40 mg, 0.05 mmol) and CsF (76 mg, 0.50 mmol) in DMF (2 mL) was stirred at 25° C. for 16 h. The resulting mixture was filtered, the filtrate was purified by pre-HPLC (10 mmol/L NH₄HCO₃ in water/MeCN) to afford title product (4 mg, yield: 12%) as light-yellow solid.

LCMS: (M+H)⁺=627.2

¹H NMR: (400 MHz, CD₃OD) δ 8.13-8.03 (m, 2H), 7.68-7.52 (m, 2H), 7.47-7.37 (m, 1H), 5.31-5.18 (m, 1H), 4.96-4.79 (m, 1H), 4.64-4.48 (m, 2H), 4.09-4.03 (m, 1H), 3.81-3.49 (m, 3H), 3.48-3.33 (m, 1H), 3.25-3.17 (m, 2H), 3.16-3.08 (m, 2H), 3.03-2.90 (m, 2H), 2.83-2.74 (m, 1H), 2.62-2.42 (m, 2H), 2.12-1.98 (m, 2H), 1.93-1.83 (m, 2H), 1.82-1.76 (m, 1H), 1.65-1.59 (m, 1H), 1.59-1.54 (m, 3H).

### Example 301a and 301b

### 2-amino-4-((5aS,6S,9R)-3-chloro-1-fluoro-14-(((S)-2-methylenetetrahydro-1H-pyrrolizin-7a(5H)-yl)methyl)-13-oxo-5a,6,7,8,9,10,13,14-octahydro-5H-6,9-epiminoazepino[2′,1′:3,4][1,4]oxazepino[5,6,7-de]quinazolin-2-yl)-7-fluorobenzo[b]thiophene-3-carbonitrile

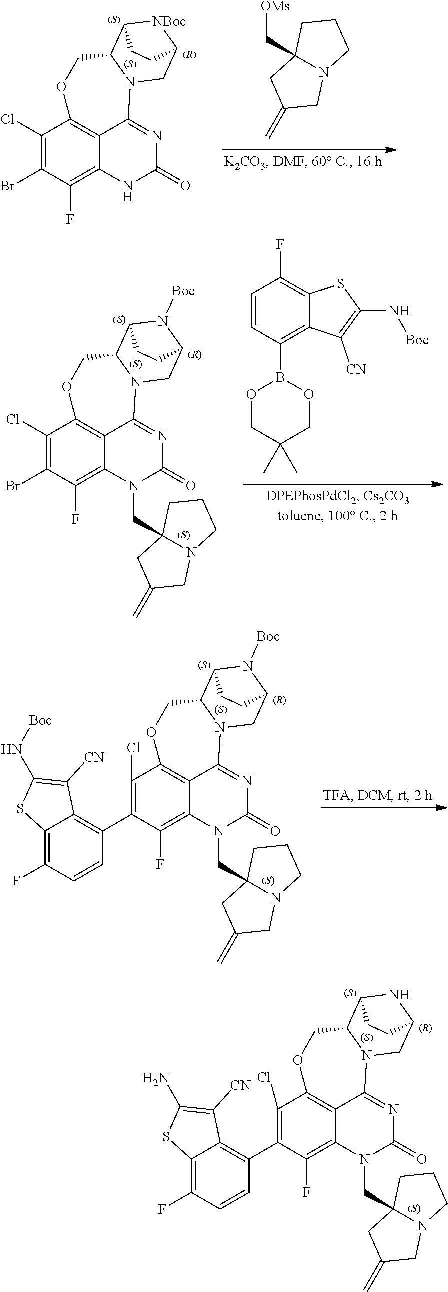

### Step 1: tert-butyl(5aS,6S,9R)-2-bromo-3-chloro-1-fluoro-14-(((S)-2-methylenetetrahydro-1H-pyrrolizin-7a(5H)-yl)methyl)-13-oxo-5a,6,7,8,9,10,13,14-octahydro-5H-6,9-epiminoazepino[2′,1′:3,4][1,4]oxazepino[5,6,7-de]quinazoline-15-carboxylate

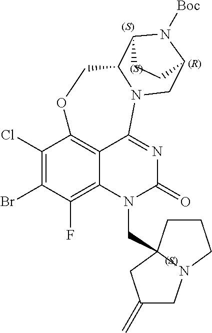

A solution of tert-butyl (5aS,6S,9R)-2-bromo-3-chloro-1-fluoro-13-oxo-5a,6,7,8,9,10,13,14-octahydro-5H-6,9-epiminoazepino[2′,1′:3,4][1,4]oxazepino[5,6,7-de]quinazoline-15-carboxylate (500 mg, 0.97 mmol), (S)-(2-methylenetetrahydro-1H-pyrrolizin-7a(5H)-yl)methyl methanesulfonate (942 mg, 3.88 mmol) and K₂CO₃ (403 mg, 2.92 mmol) in DMF (10 mL) and the reaction was stirred for 16 hours at 60° C. under N₂ atmosphere. The reaction was diluted with H₂O (50 mL) and extracted with EA (50 mL×3). The combined organic layers were washed with brine (50 mL×2), dried over anhydrous Na₂SO₄. After filtration, the filtrate was concentrated and the residue purified by silica gel column (DCM:MeOH=10:1) to give the Product (150 mg, yield: 23%) as a yellow solid.

LCMS: (M+H)⁺=650.0

### Step 2: tert-butyl(5aS,6S,9R)-2-(2-((tert-butoxycarbonyl)amino)-3-cyano-7-fluorobenzo[b]thiophen-4-yl)-3-chloro-1-fluoro-14-(((S)-2-methylenetetrahydro-1H-pyrrolizin-7a(5H)-yl)methyl)-13-oxo-5a,6,7,8,9,10,13,14-octahydro-5H-6,9-epiminoazepino[2′,1′:3,4][1,4]oxazepino[5,6,7-de]quinazoline-15-carboxylate

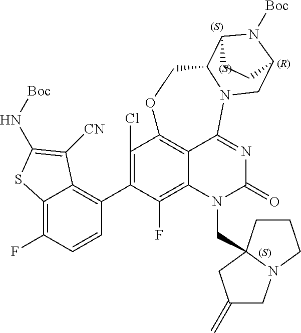

A mixture of tert-butyl(5aS,6S,9R)-2-bromo-3-chloro-1-fluoro-14-(((S)-2-methylenetetrahydro-1H-pyrrolizin-7a(5H)-yl)methyl)-13-oxo-5a,6,7,8,9,10,13,14-octahydro-5H-6,9-epiminoazepino[2′,1′:3,4][1,4]oxazepino[5,6,7-de]quinazoline-15-carboxylate (140 mg, 0.22 mmol), tert-butyl (3-cyano-4-(5,5-dimethyl-1,3,2-dioxaborinan-2-yl)-7-fluorobenzo[b]thiophen-2-yl)carbamate (174 mg, 0.44 mmol), DPEPhosPdCl₂ (46 mg, 0.06 mmol) and Cs₂CO₃ (210 mg, 0.65 mmol) in toluene (5 mL) was stirred at 100° C. for 2 hours under N₂ atmosphere. The reaction was diluted with H₂O (30 mL) and extracted with EA (50 mL×3). The combined organic layers were washed with brine (50 mL×2), dried over anhydrous Na₂SO₄. After filtration, the filtrate was concentrated and the residue purified by C18 column chromatography eluted with (0.1% FA in H₂O/ACN) to give the Product (47 mg) as a yellow solid. The stereoisomers were separated by chiral SFC to afford isomer 1 (16 mg, yield: 8%) as a yellow solid and isomer 2 (12 mg, yield: 6%) as a yellow solid.

LCMS: (M+H)⁺=862.2

### Step 3: 2-amino-4-((5aS,6S,9R)-3-chloro-1-fluoro-14-(((S)-2-methylenetetrahydro-1H-pyrrolizin-7a(5H)-yl)methyl)-13-oxo-5a,6,7,8,9,10,13,14-octahydro-5H-6,9-epiminoazepino[2′,1′:3,4][1,4]oxazepino[5,6,7-de]quinazolin-2-yl)-7-fluorobenzo[b]thiophene-3-carbonitrile

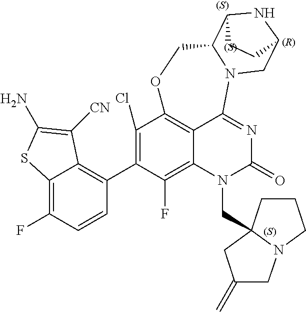

To a solution of tert-butyl(5aS,6S,9R)-2-(2-((tert-butoxycarbonyl)amino)-3-cyano-7-fluorobenzo[b]thiophen-4-yl)-3-chloro-1-fluoro-14-(((S)-2-methylenetetrahydro-1H-pyrrolizin-7a(5H)-yl)methyl)-13-oxo-5a,6,7,8,9,10,13,14-octahydro-5H-6,9-epiminoazepino[2′,1′:3,4][1,4]oxazepino[5,6,7-de]quinazoline-15-carboxylate (isomer 1) (16 mg, 0.02 mmol) in DCM (1.5 mL) was added TFA (0.5 mL). The reaction was stirred for 2 hours at 25° C. The resulting mixture was concentrated and purified by pre-HPLC (10 mmol/L NH₄HCO₃ in water/MeCN) to afford Example 310a (4 mg, yield: 32%) as white solid.

LCMS: (M+H)⁺=662.5

Example 310a ¹H NMR: (400 MHz, CD₃OD) δ 7.20-7.15 (m, 1H), 7.05-6.98 (m, 1H), 5.07-5.01 (m, 1H), 4.91-4.89 (m, 1H), 4.83-4.80 (m, 1H), 4.60-4.54 (m, 1H), 4.39-4.31 (m, 1H), 4.19-4.13 (m, 1H), 3.74-3.62 (m, 2H), 3.59-3.52 (m, 2H), 3.52-3.46 (m, 1H), 3.43-3.37 (m, 1H), 3.35-3.33 (m, 1H), 3.26-3.21 (m, 1H), 3.07-2.94 (m, 3H), 2.83-2.76 (m, 1H), 2.51-2.37 (m, 1H), 2.19-2.08 (m, 1H), 1.96-1.87 (m, 3H), 1.82-1.72 (m, 1H), 1.57-1.49 (m, 1H).

To a solution of tert-butyl(5aS,6S,9R)-2-(2-((tert-butoxycarbonyl)amino)-3-cyano-7-fluorobenzo[b]thiophen-4-yl)-3-chloro-1-fluoro-14-(((S)-2-methylenetetrahydro-1H-pyrrolizin-7a(5H)-yl)methyl)-13-oxo-5a,6,7,8,9,10,13,14-octahydro-5H-6,9-epiminoazepino[2′,1′:3,4][1,4]oxazepino[5,6,7-de]quinazoline-15-carboxylate (isomer 2) (12 mg, 0.01 mmol) in DCM (1.5 mL) was added TFA (0.5 mL). The reaction was stirred for 2 hours at 25° C. The resulting mixture was concentrated and purified by pre-HPLC (10 mmol/L NH₄HCO₃ in water/MeCN) to afford Example 310b (2 mg, yield: 21%) as white solid.

LCMS: (M+H)⁺=662.5

Example 310b ¹H NMR: (400 MHz, CD₃OD) δ 7.21-7.15 (m, 1H), 7.05-6.99 (m, 1H), 4.93-4.89 (m, 3H), 4.59-4.53 (m, 1H), 4.43-4.36 (m, 1H), 4.11-4.06 (m, 1H), 3.73-3.62 (m, 2H), 3.60-3.49 (m, 3H), 3.48-3.40 (m, 2H), 3.35-3.33 (m, 1H), 3.08-2.94 (m, 3H), 2.82-2.74 (m, 1H), 2.52-2.38 (m, 1H), 2.20-2.08 (m, 1H), 2.07-1.98 (m, 1H), 1.92-1.76 (m, 3H), 1.58-1.49 (in, 1H).

### Example 365a and 365b

### (5S,5aS,6S,9R)-13-(2-(dimethylamino)-3-hydroxy-2-methylpropyl)-2-(8-ethynyl-7-fluoronaphthalen-1-yl)-1-fluoro-5-methyl-5a,6,7,8,9,10-hexahydro-5H-4-oxa-3,10a,11,13,14-pentaaza-6,9-methanonaphtho[1,8-ab]heptalen-12(13H)-one

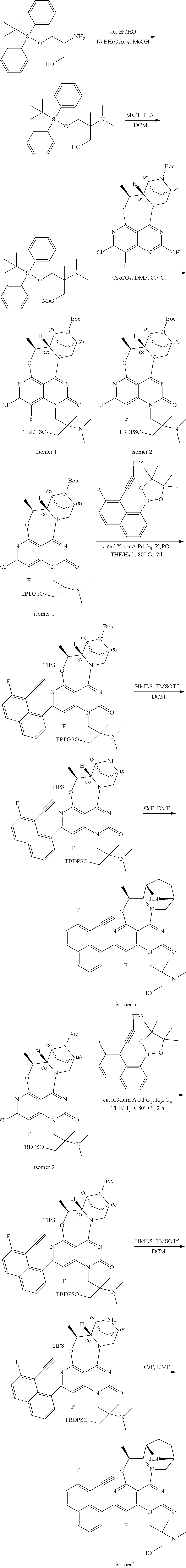

### Step 1. 3-((tert-butyldiphenylsilyl)oxy)-2-(dimethylamino)-2-methylpropan-1-ol

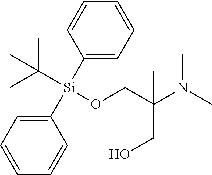 [SMILES: CN(C)C(C)(CO)CO[Si](c1ccccc1)(c1ccccc1)C(C)(C)C]

To a stirred solution of 2-amino-3-((tert-butyldiphenylsilyl)oxy)-2-methylpropan-1-ol (1.00 g, 2.91 mmol) in MeOH (10 mL) was added HCHO (5 mL, 37% wt in water) and NaBH(OAc)₃ (1.85 g, 8.73 mmol) at 0° C. After stirring at room temperature for 12 hours, the reaction was poured into water (30 mL) and extracted with DCM (30 mL×2). The combined organic phases were dried over Na₂SO₄ and evaporated to dryness. The residue was purified by column chromatography on silica gel eluted with DCM/MeOH=15:1) to give the desired product (800 mg, 74% yield) as a yellow oil.

LC/MS (ESI) (m/z): 372 [M+H]⁺.

### Step 2. 3-((tert-butyldiphenylsilyl)oxy)-2-(dimethylamino)-2-methylpropyl methanesulfonate

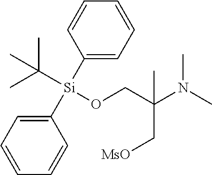 [SMILES: CN(C)C(C)(CO[Si](c1ccccc1)(c1ccccc1)C(C)(C)C)COS(C)(=O)=O]

To a stirred solution of 3-((tert-butyldiphenylsilyl)oxy)-2-(dimethylamino)-2-methylpropan-1-ol (722 mg, 1.94 mmol) and Et₃N (590 mg, 5.83 mmol) in DCM (10 mL) was added dropwise a solution of MsCl (445 mg, 3.89 mmol) in DCM (1 mL) at 0° C. under N₂. The reaction was stirred at 0° C. for 1 h, TLC (DCM:MeOH=10:1) indicated the reaction was complete. The reaction mixture was poured into water (20 mL) and extracted with DCM (10 mL). The organic phase was dried over Na₂SO₄ and evaporated to dryness to give the desired product (873 mg, crude) as a yellow oil.

LC/MS (ESI) (m/z): 450 [M+H]⁺.

### Step 3. tert-butyl (5S,5aS,6S,9R)-13-(3-((tert-butyldiphenylsilyl)oxy)-2-(dimethylamino)-2-methylpropyl)-2-chloro-1-fluoro-5-methyl-12-oxo-5a,6,7,8,9,10,12,13-octahydro-5H-4-oxa-3,10a,11,13,14-pentaaza-6,9-methanonaphtho[1,8-ab]heptalene-14-carboxylate

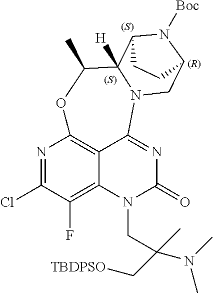

To a mixture of tert-butyl (5S,5aS,6S,9R)-2-chloro-1-fluoro-12-hydroxy-5-methyl-5a,6,7,8,9,10-hexahydro-5H-4-oxa-3,10a,11,13,14-pentaaza-6,9-methanonaphtho[1,8-ab]heptalene-14-carboxylate (340 mg, 0.75 mmol) and Cs₂CO₃ (735 mg, 2.26 mmol) in DMF (10 mL) was added a solution of 3-((tert-butyldiphenylsilyl)oxy)-2-(dimethylamino)-2-methylpropyl methanesulfonate (1.02 g, 2.26 mmol) in DMF (2 mL) at room temperature. The reaction was stirred at 80° C. for 2 hours. LCMS showed the reaction was complete. The reaction mixture was cooled to room temperature, then poured into water (40 mL) and extracted with EtOAc (15 mL×3). The combined organic phases were washed with brine (30 mL), dried over anhydrous Na₂SO₄ and concentrated to dryness. The residue was purified by column chromatography on silica gel (DCM:MeOH=15:1) to give 300 mg of product (mixture of two isomers).

The products was further purified by prep-TLC (DCM:MeOH=15:1, the maximum amount of sample loading on TLC did not exceed 15 mg) to give the desired products (isomer 1: the first eluting isomer, 52 mg, isomer 2: the second eluting isomer, 50 mg) as a yellow solid.

LC/MS (ESI) (m/z): 805 [M+H]⁺.

### Step 4. tert-butyl (5S,5aS,6S,9R)-13-(3-((tert-butyldiphenylsilyl)oxy)-2-(dimethylamino)-2-methylpropyl)-1-fluoro-2-(7-fluoro-8-((triisopropylsilyl)ethynyl)naphthalen-1-yl)-5-methyl-12-oxo-5a,6,7,8,9,10,12,13-octahydro-5H-4-oxa-3,10a,11,13,14-pentaaza-6,9-methanonaphtho[1,8-ab]heptalene-14-carboxylate

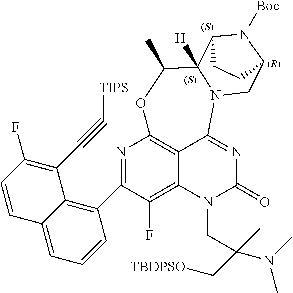

To a mixture of tert-butyl (5S,5aS,6S,9R)-13-(3-((tert-butyldiphenylsilyl)oxy)-2-(dimethylamino)-2-methylpropyl)-2-chloro-1-fluoro-5-methyl-12-oxo-5a,6,7,8,9,10,12,13-octahydro-5H-4-oxa-3,10a,11,13,14-pentaaza-6,9-methanonaphtho[1,8-ab]heptalene-14-carboxylate (isomer 1: 52 mg, 0.07 mmol) and ((2-fluoro-8-(4,4,5,5-tetramethyl-1,3,2-dioxaborolan-2-yl)naphthalen-1-yl)ethynyl)triisopropylsilane (58 mg, 0.13 mmol) in THF (2 mL) and water (0.4 mL) were added K₃PO₄ (41 mg, 0.19 mmol) and CataCXium A Pd G_{S} (9 mg, 0.013 mmol). The mixture was degassed for three times under N₂ atmosphere and stirred at 80° C. for 1 hour. LCMS showed the reaction was complete. The reaction mixture was diluted with EtOAc (10 mL), washed with water (10 mL) and brine (10 mL), dried over anhydrous Na₂SO₄ and concentrated to dryness. The residue was purified by prep-TLC (DCM:MeOH:NH₃OH=15:1:0.1) to give the desired product (60 mg, 84.8% yield) as a yellow solid.

LC/MS (ESI) (m/z): 1095.6 [M+H]⁺.

### Step 5. (5S,5aS,6S,9R)-13-(3-((tert-butyldiphenylsilyl)oxy)-2-(dimethylamino)-2-methylpropyl)-1-fluoro-2-(7-fluoro-8-((triisopropylsilyl)ethynyl)naphthalen-1-yl)-5-methyl-5a,6,7,8,9,10-hexahydro-5H-4-oxa-3,10a,11,13,14-pentaaza-6,9-methanonaphtho[1,8-ab]heptalen-12(13H)-one

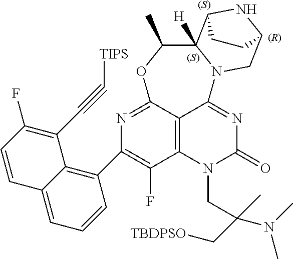

To a solution of tert-butyl (5S,5aS,6S,9R)-13-(3-((tert-butyldiphenylsilyl)oxy)-2-(dimethylamino)-2-methylpropyl)-1-fluoro-2-(7-fluoro-8-((triisopropylsilyl)ethynyl)naphthalen-1-yl)-5-methyl-12-oxo-5a,6,7,8,9,10,12,13-octahydro-5H-4-oxa-3,10a,11,13,14-pentaaza-6,9-methanonaphtho[1,8-ab]heptalene-14-carboxylate (60 mg, 0.06 mmol) in DCM (2 mL) were added HMDS (0.3 mL) and TMSOTf (0.15 mL) at 0° C. under N₂. The reaction was stirred at 0° C. for 30 mins. TLC (DCM:MeOH=10:1) indicated the reaction was complete. The reaction mixture was poured into sat. NaHCO₃(aq.) (10 mL) and extracted with DCM (5 mL). The organic phase was washed with brine (10 mL), dried over anhydrous Na₂SO₄ and concentrated to give the desired product (54 mg) as a yellow oil which was used in the next step without further purification.

LCMS(ESI) (m/z): 995.5 [M+H]⁺.

### Step 6. (5S,5aS,6S,9R)-13-(2-(dimethylamino)-3-hydroxy-2-methylpropyl)-2-(8-ethynyl-7-fluoronaphthalen-1-yl)-1-fluoro-5-methyl-5a,6,7,8,9,10-hexahydro-5H-4-oxa-3,10a,11,13,14-pentaaza-6,9-methanonaphtho[1,8-ab]heptalen-12(13H)-one

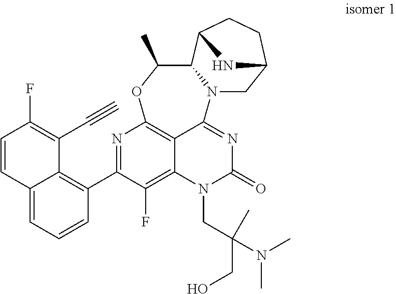 [SMILES: C#Cc1c(F)ccc2cccc(-c3nc4c5c(nc(=O)n(CC(C)(CO)N(C)C)c5c3F)N3C[C@H]5CC[C@H](N5)[C@H]3[C@H](C)O4)c12]

To a solution of (5S,5aS,6S,9R)-13-(3-((tert-butyldiphenylsilyl)oxy)-2-(dimethylamino)-2-methylpropyl)-1-fluoro-2-(7-fluoro-8-((triisopropylsilyl)ethynyl)naphthalen-1-yl)-5-methyl-5a,6,7,8,9,10-hexahydro-5H-4-oxa-3,10a,11,13,14-pentaaza-6,9-methanonaphtho[1,8-ab]heptalen-12(13H)-one (54 mg) in DMF (3 mL) was added caesium fluoride (330 mg, 2.17 mmol). The mixture was stirred at room temperature for 48 hours. LCMS showed the reaction was complete. The reaction mixture was filtered and purified by prep-HPLC (chromatographic column: YMC-Actus Triart C18 150*20 mm; mobile phase A: 0.1% NH₃ in water, mobile phase B: MeCN; gradient: 20% B to 95% B in 18 min; flow rate of 25 ml/min; Wave length: 220 nm/254 nm; Target Retention time: 9 MIN (75% MECN)) to give Example 365a (4 mg, 12% yield) as a white solid.

LC/MS (ESI) (m/z): 601 [M+H]⁺.

¹H NMR (400 MHz, CD₃OD) δ 8.17-7.96 (m, 2H), 7.70-7.52 (m, 2H), 7.49-7.29 (m, 1H), 5.48-5.31 (m, 1H), 4.62-4.48 (m, 1H), 4.42-4.26 (m, 2H), 4.09 (d, J=8.8 Hz, 1H), 3.73-3.44 (m, 3H), 3.25-3.09 (m, 1H), 2.66-2.49 (m, 2H), 2.37 (s, 6H), 2.14-1.98 (m, 1H), 1.92-1.71 (m, 3H), 1.64-1.51 (m, 3H), 1.31 (s, 3H).

### Step 7. tert-butyl (5S,5aS,6S,9R)-13-(3-((tert-butyldiphenylsilyl)oxy)-2-(dimethylamino)-2-methylpropyl)-1-fluoro-2-(7-fluoro-8-((triisopropylsilyl)ethynyl)naphthalen-1-yl)-5-methyl-12-oxo-5a,6,7,8,9,10,12,13-octahydro-5H-4-oxa-3,10a,11,13,14-pentaaza-6,9-methanonaphtho[1,8-ab]heptalene-14-carboxylate

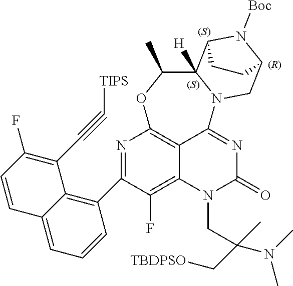

To a mixture of tert-butyl (5S,5aS,6S,9R)-13-(3-((tert-butyldiphenylsilyl)oxy)-2-(dimethylamino)-2-methylpropyl)-2-chloro-1-fluoro-5-methyl-12-oxo-5a,6,7,8,9,10,12,13-octahydro-5H-4-oxa-3,10a,11,13,14-pentaaza-6,9-methanonaphtho[1,8-ab]heptalene-14-carboxylate (isomer 2: 50 mg, 0.07 mmol) and ((2-fluoro-8-(4,4,5,5-tetramethyl-1,3,2-dioxaborolan-2-yl)naphthalen-1-yl)ethynyl)triisopropylsilane (58 mg, 0.13 mmol) in THF (2 mL) and water (0.4 mL) were added K₃PO₄ (41 mg, 0.19 mmol) and CataCXium A Pd G₃ (9 mg, 0.013 mmol). The mixture was degassed for three times under N₂ atmosphere and stirred at 80° C. for 1 hour. LCMS showed the reaction was complete. The reaction mixture was diluted with EtOAc (10 mL), washed with water (10 mL) and brine (10 mL), dried over anhydrous Na₂SO₄ and concentrated to dryness. The residue was purified by prep-TLC (DCM:MeOH:NH₃OH=15:1:0.1) to give the desired product (60 mg, 84.8% yield) as a yellow solid.

LC/MS (ESI) (m/z): 1095.6 [M+H]f.

### Step 8. (5S,5aS,6S,9R)-13-(3-((tert-butyldiphenylsilyl)oxy)-2-(dimethylamino)-2-methylpropyl)-1-fluoro-2-(7-fluoro-8-((triisopropylsilyl)ethynyl)naphthalen-1-yl)-5-methyl-5a,6,7,8,9,10-hexahydro-5H-4-oxa-3,10a,11,13,14-pentaaza-6,9-methanonaphtho[1,8-ab]heptalen-12(13H)-one

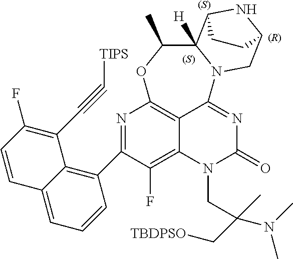

To a solution of tert-butyl (5S,5aS,6S,9R)-13-(3-((tert-butyldiphenylsilyl)oxy)-2-(dimethylamino)-2-methylpropyl)-1-fluoro-2-(7-fluoro-8-((triisopropylsilyl)ethynyl)naphthalen-1-yl)-5-methyl-12-oxo-5a,6,7,8,9,10,12,13-octahydro-5H-4-oxa-3,10a,11,13,14-pentaaza-6,9-methanonaphtho[1,8-ab]heptalene-14-carboxylate (50 mg, 0.04 mmol) in DCM (3 mL) was added HMDS (0.4 mL) and TMSOTf (0.2 mL) at 0° C. under N₂, the reaction was stirred at 0° C. for 30 mins. The reaction was complete detected by TLC (DCM/MeOH=10:1). The reaction mixture was poured into sat. NaHCO₃ (10 mL) and extracted with DCM (5 mL×3). The combined organic phases were washed with brine (20 mL), dried over anhydrous Na₂SO₄ and concentrated to give the desired product (40 mg, 89% yield) as a yellow solid.

LCMS(ESI) (m/z): 995 [M+H]⁺.

### Step 9. (5S,5aS,6S,9R)-13-(2-(dimethylamino)-3-hydroxy-2-methylpropyl)-2-(8-ethynyl-7-fluoronaphthalen-1-yl)-1-fluoro-5-methyl-5a,6,7,8,9,10-hexahydro-5H-4-oxa-3,10a,11,13,14-pentaaza-6,9-methanonaphtho[1,8-ab]heptalen-12(13H)-one

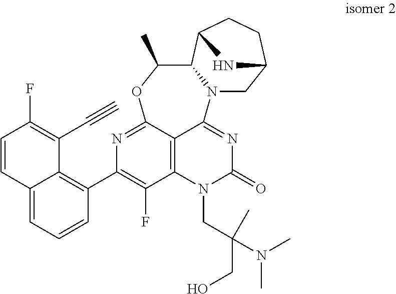 [SMILES: C#Cc1c(F)ccc2cccc(-c3nc4c5c(nc(=O)n(CC(C)(CO)N(C)C)c5c3F)N3C[C@H]5CC[C@H](N5)[C@H]3[C@H](C)O4)c12]

To a solution of (5S,5aS,6S,9R)-13-(3-((tert-butyldiphenylsilyl)oxy)-2-(dimethylamino)-2-methylpropyl)-1-fluoro-2-(7-fluoro-8-((triisopropylsilyl)ethynyl)naphthalen-1-yl)-5-methyl-5a,6,7,8,9,10-hexahydro-5H-4-oxa-3,10a,11,13,14-pentaaza-6,9-methanonaphtho[1,8-ab]heptalen-12(13H)-one (40 mg, 0.04 mmol) in DMF (5 mL) was added caesium fluoride (242 mg, 1.6 mmol) and the reaction was stirred at room temperature for 48 hours. LCMS showed the reaction was complete. The mixture was filtered and purified by prep-HPLC (chromatographic column: YMC-Actus Triart C18 150*20 mm; mobile phase A: 0.1% NH₃ in water, mobile phase B: MeCN; gradient: 20% B to 95% B in 18 min; flow rate of 25 ml/min; Wave length: 220 nm/254 nm; Target Retention time: 9 MIN (75% MECN)) to Example 365b (4.8 mg, 20% yield) as a white solid.

LC/MS (ESI) (m/z): 601 [M+H]⁺.

¹H NMR (400 MHz, CD₃OD) δ 8.16-7.98 (m, 2H), 7.71-27.51 (m, 2H), 7.43 (q, J=9.1 Hz, 1H), 5.40 (d, J=13.3 Hz, 1H), 4.54 (dd, J=8.6, 6.4 Hz, 1H), 4.43-4.29 (m, 2H), 4.09 (d, J=8.4 Hz, 1H), 3.79-3.48 (m, 3H), 3.23-3.16 (m, 1H), 2.66-2.50 (m, 2H), 2.38 (s, 6H), 2.08 (d, J=5.8 Hz, 1H), 1.92-1.71 (m, 3H), 1.62-1.53 (m, 3H), 1.32 (d, J=3.1 Hz, 3H).

The following compounds were prepared according to the above-described methods using different starting materials.

| | | | MS |
|---|---|---|---|
| Exp. No. | Structure | Name | m/z |
| 2 | 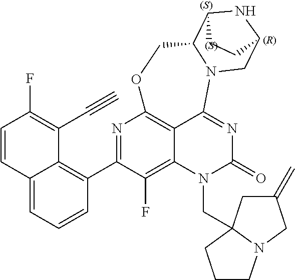 | (5aS,6S,9R)-13-((2,6- dimethylenetetrahydro-1H- pyrrolizin-7a(5H)-yl)methyl)-2- (8-ethynyl-7-fluoronaphthalen- 1-yl)-1-fluoro-5a,6,7,8,9,10- hexahydro-5H-4-oxa- 3,10a,11,13,14-pentaaza-6,9- methanonaphtho[1,8- ab]heptalen-12(13H)-one | 607 [M + H]⁺ |
| 3 | 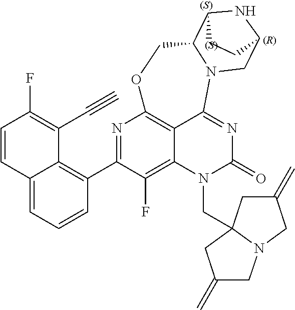 | (5aS,6S,9R)-2-(8-ethynyl-7- fluoronaphthalen-1-yl)-1-fluoro- 13-(((2R,7aS)-2- fluorotetrahydro-1H-pyrrolizin- 7a(5H)-yl)methyl)-5a,6,7,8,9,10- hexahydro-5H-4-oxa- 3,10a,11,13,14-pentaaza-6,9- methanonaphtho[1,8- ab]heptalen-12(13H)-one | 619 [M + H]⁺ |
| 4 | 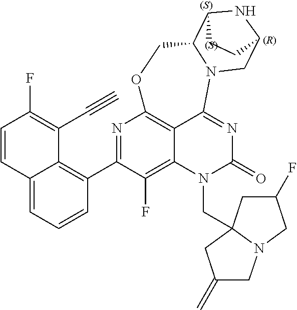 | (5aS,6S,9R)-2-(8-ethynyl-7- fluoronaphthalen-1-yl)-1-fluoro- 13-((2- (fluoromethylene)tetrahydro- 1H-pyrrolizin-7a(5H)- yl)methyl)-5a,6,7,8,9,10- hexahydro-5H-4-oxa- 3,10a,11,13,14-pentaaza-6,9- methanonaphtho[1,8- ab]heptalen-12(13H)-one | 625 [M + H]⁺ |
| 5 | 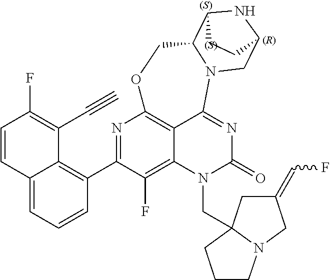 | (5aS,6S,9R)-2-(8-ethynyl-7- fluoronaphthalen-1-yl)-1-fluoro- 13-((2-fluoro-6- methylenetetrahydro-1H- pyrrolizin-7a(5H)-yl)methyl)- 5a,6,7,8,9,10-hexahydro-5H-4- oxa-3,10a,11,13,14-pentaaza- 6,9-methanonaphtho[1,8- ab]heptalen-12(13H)-one | 625 [M + H]⁺ |
| 6 | 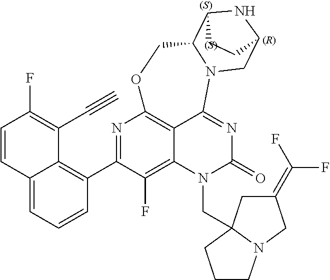 | (5aS,6S,9R)-13-((2- (difluoromethylene)tetrahydro- 1H-pyrrolizin-7a(5H)- yl)methyl)-2-(8-ethynyl-7- fluoronaphthalen-1-yl)-1-fluoro- 5a,6,7,8,9,10-hexahydro-5H-4- oxa-3,10a,11,13,14-pentaaza- 6,9-methanonaphtho[1,8- ab]heptalen-12(13H)-one | 643 [M + H]⁺ |
| 7 | 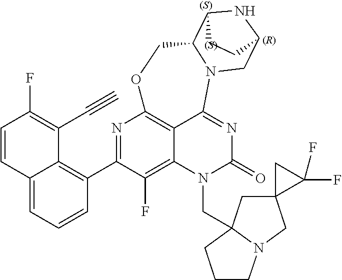 | (5aS,6S,9R)-13-((2,2- difluorodihydro-1′H,3′H- spiro[cyclopropane-1,2′- pyrrolizin]-7a′(5′H)-yl)methyl)- 2-(8-ethynyl-7- fluoronaphthalen-1-yl)-1-fluoro- 5a,6,7,8,9,10-hexahydro-5H-4- oxa-3,10a,11,13,14-pentaaza- 6,9-methanonaphtho[1,8- ab]heptalen-12(13H)-one | 657 [M + H]⁺ |
| 8 | 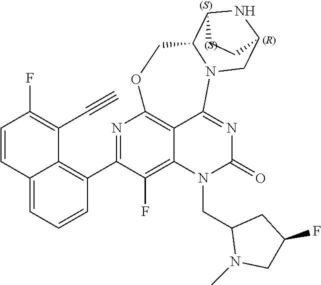 | (5aS,6S,9R)-2-(8-ethynyl-7- fluoronaphthalen-1-yl)-1-fluoro- 13-(((4R)-4-fluoro-1- methylpyrrolidin-2-yl)methyl)- 5a,6,7,8,9,10-hexahydro-5H-4- oxa-3,10a,11,13,14-pentaaza- 6,9-methanonaphtho[1,8- ab]heptalen-12(13H)-one | 587 [M + H]⁺ |
| 9 | 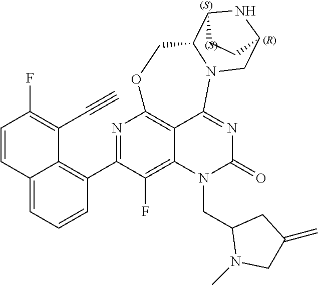 | (5aS,6S,9R)-2-(8-ethynyl-7- fluoronaphthalen-1-yl)-1-fluoro- 13-((1-methyl-4- methylenepyrrolidin-2- yl)methyl)-5a,6,7,8,9,10- hexahydro-5H-4-oxa- 3,10a,11,13,14-pentaaza-6,9- methanonaphtho[1,8- ab]heptalen-12(13H)-one | 581 [M + H]⁺ |
| 10 | 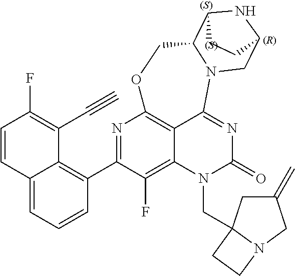 | (5aS,6S,9R)-2-(8-ethynyl-7- fluoronaphthalen-1-yl)-1-fluoro- 13-((3-methylene-1- azabicyclo[3.2.0]heptan-5- yl)methyl)-5a,6,7,8,9,10- hexahydro-5H-4-oxa- 3,10a,11,13,14-pentaaza-6,9- methanonaphtho[1,8- ab]heptalen-12(13H)-one | 593 [M + H]⁺ |
| 11 | 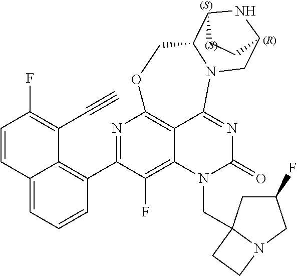 | (5aS,6S,9R)-2-(8-ethynyl-7- fluoronaphthalen-1-yl)-1-fluoro- 13-(((3R)-3-fluoro-1- azabicyclo[3.2.0]heptan-5- yl)methyl)-5a,6,7,8,9,10- hexahydro-5H-4-oxa- 3,10a,11,13,14-pentaaza-6,9- methanonaphtho[1,8- ab]heptalen-12(13H)-one | 599 [M + H]⁺ |
| 12 | 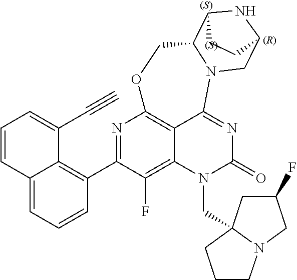 | (5aS,6S,9R)-2-(8- ethynylnaphthalen-1-yl)-1- fluoro-13-((2- methylenetetrahydro-1H- pyrrolizin-7a(5H)-yl)methyl)- 5a,6,7,8,9,10-hexahydro-5H-4- oxa-3,10a,11,13,14-pentaaza- 6,9-methanonaphtho[1,8- ab]heptalen-12(13H)-one | 595 [M + H]⁺ |
| 13 | 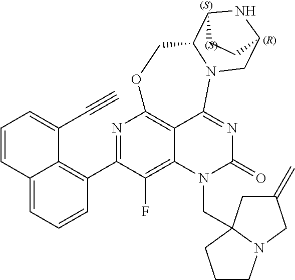 | (5aS,6S,9R)-13-((2,6- dimethylenetetrahydro-1H- pyrrolizin-7a(5H)-yl)methyl)-2- (8-ethynylnaphthalen-1-yl)-1- fluoro-5a,6,7,8,9,10-hexahydro- 5H-4-oxa-3,10a,11,13,14- pentaaza-6,9- methanonaphtho[1,8- ab]heptalen-12(13H)-one | 589 [M + H]⁺ |
| 14 | 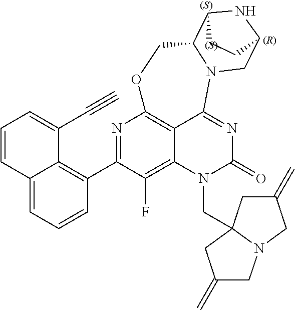 | (5aS,6S,9R)-2-(8- ethynylnaphthalen-1-yl)-1- fluoro-13-(((2R,7aS)-2- fluorotetrahydro-1H-pyrrolizin- 7a(5H)-yl)methyl)-5a,6,7,8,9,10- hexahydro-5H-4-oxa- 3,10a,11,13,14-pentaaza-6,9- methanonaphtho[1,8- ab]heptalen-12(13H)-one | 601 [M + H]⁺ |
| 15 | 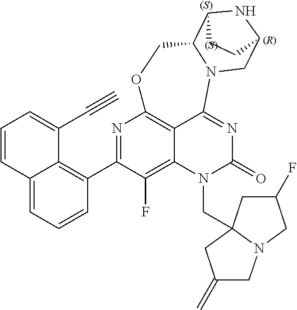 | (5aS,6S,9R)-2-(8- ethynylnaphthalen-1-yl)-1- fluoro-13-((2- (fluoromethylene)tetrahydro- 1H-pyrrolizin-7a(5H)- yl)methyl)-5a,6,7,8,9,10- hexahydro-5H-4-oxa- 3,10a,11,13,14-pentaaza-6,9- methanonaphtho[1,8- ab]heptalen-12(13H)-one | 607 [M + H]⁺ |
| 16 | 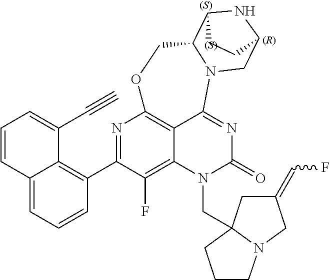 | (5aS,6S,9R)-2-(8- ethynylnaphthalen-1-yl)-1- fluoro-13-((2-fluoro-6- methylenetetrahydro-1H- pyrrolizin-7a(5H)-yl)methyl)- 5a,6,7,8,9,10-hexahydro-5H-4- oxa-3,10a,11,13,14-pentaaza- 6,9-methanonaphtho[1,8- ab]heptalen-12(13H)-one | 607 [M + H]⁺ |
| 17 | 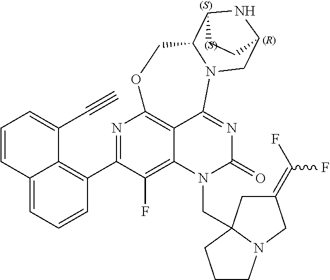 | (5aS,6S,9R)-13-((2- (difluoromethylene)tetrahydro- 1H-pyrrolizin-7a(5H)- yl)methyl)-2-(8- ethynylnaphthalen-1-yl)-1- fluoro-5a,6,7,8,9,10-hexahydro- 5H-4-oxa-3,10a,11,13,14- pentaaza-6,9- methanonaphtho[1,8- ab]heptalen-12(13H)-one | 625 [M + H]⁺ |
| 18 | 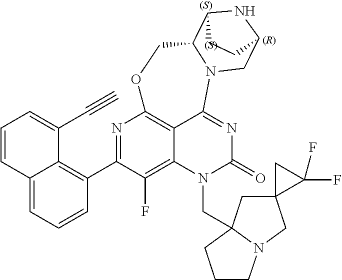 | (5aS,6S,9R)-13-((2,2- difluorodihydro-1′H,3′H- spiro[cyclopropane-1,2′- pyrrolizin]-7a′(5′H)-yl)methyl)- 2-(8-ethynylnaphthalen-1-yl)-1- fluoro-5a,6,7,8,9,10-hexahydro- 5H-4-oxa-3,10a,11,13,14- pentaaza-6,9- methanonaphtho[1,8- ab]heptalen-12(13H)-one | 639 [M + H]⁺ |
| 19 | 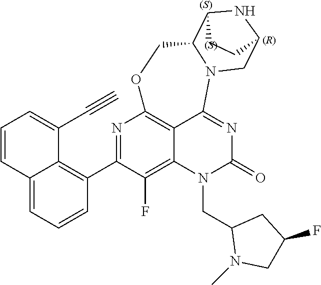 | (5aS,6S,9R)-2-(8- ethynylnaphthalen-1-yl)-1- fluoro-13-(((4R)-4-fluoro-1- methylpyrrolidin-2-yl)methyl)- 5a,6,7,8,9,10-hexahydro-5H-4- oxa-3,10a,11,13,14-pentaaza- 6,9-methanonaphtho[1,8- ab]heptalen-12(13H)-one | 569 [M + H]⁺ |
| 20 | 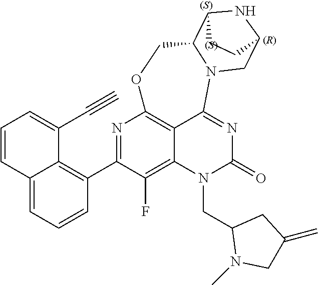 | (5aS,6S,9R)-2-(8- ethynylnaphthalen-1-yl)-1- fluoro-13-((1-methyl-4- methylenepyrrolidin-2- yl)methyl)-5a,6,7,8,9,10- hexahydro-5H-4-oxa- 3,10a,11,13,14-pentaaza-6,9- methanonaphtho[1,8- ab]heptalen-12(13H)-one | 563 [M + H]⁺ |
| 21 | 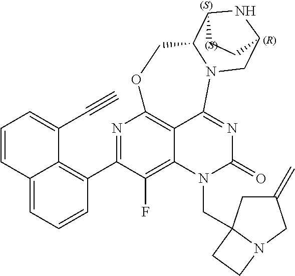 | (5aS,6S,9R)-2-(8- ethynylnaphthalen-1-yl)-1- fluoro-13-((3-methylene-1- azabicyclo[3.2.0]heptan-5- yl)methyl)-5a,6,7,8,9,10- hexahydro-5H-4-oxa- 3,10a,11,13,14-pentaaza-6,9- methanonaphtho[1,8- ab]heptalen-12(13H)-one | 575 [M + H]⁺ |
| 22 | 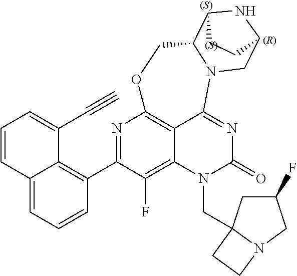 | (5aS,6S,9R)-2-(8- ethynylnaphthalen-1-yl)-1- fluoro-13-(((3R)-3-fluoro-1- azabicyclo[3.2.0]heptan-5- yl)methyl)-5a,6,7,8,9,10- hexahydro-5H-4-oxa- 3,10a,11,13,14-pentaaza-6,9- methanonaphtho[1,8- ab]heptalen-12(13H)-one | 581 [M + H]⁺ |
| 24 | 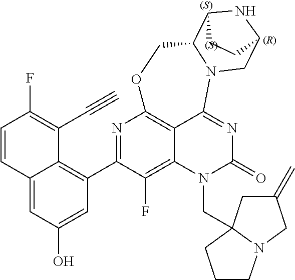 | (5aS,6S,9R)-13-((2,6- dimethylenetetrahydro-1H- pyrrolizin-7a(5H)-yl)methyl)-2- (8-ethynyl-7-fluoro-3- hydroxynaphthalen-1-yl)-1- fluoro-5a,6,7,8,9,10-hexahydro- 5H-4-oxa-3,10a,11,13,14- pentaaza-6,9- methanonaphtho[1,8- ab]heptalen-12(13H)-one | 623 [M + H]⁺ |
| 25 | 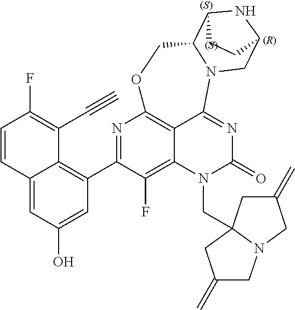 | (5aS,6S,9R)-2-(8-ethynyl-7- fluoro-3-hydroxynaphthalen-1- yl)-1-fluoro-13-(((2R,7aS)-2- fluorotetrahydro-1 H-pyrrolizin- 7a(5H)-yl)methyl)-5a,6,7,8,9,10- hexahydro-5H-4-oxa- 3,10a,11,13,14-pentaaza-6,9- methanonaphtho[1,8- ab]heptalen-12(13H)-one | 635 [M + H]⁺ |
| 26 | 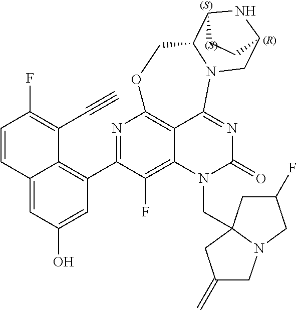 | (5aS,6S,9R)-2-(8-ethynyl-7- fluoro-3-hydroxynaphthalen-1- yl)-1-fluoro-13-((2- (fluoromethylene)tetrahydro- 1H-pyrrolizin-7a(5H)- yl)methyl)-5a,6,7,8,9,10- hexahydro-5H-4-oxa- 3,10a,11,13,14-pentaaza-6,9- methanonaphtho[1,8- ab]heptalen-12(13H)-one | 641 [M + H]⁺ |
| 27 | 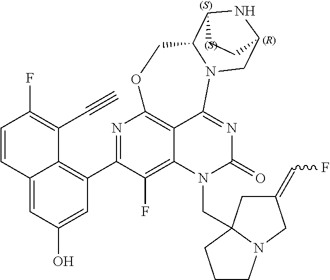 | (5aS,6S,9R)-2-(8-ethynyl-7- fluoro-3-hydroxynaphthalen-1- yl)-1-fluoro-13-((2-fluoro-6- methylenetetrahydro-1H- pyrrolizin-7a(5H)-yl)methyl)- 5a,6,7,8,9,10-hexahydro-5H-4- oxa-3,10a,11,13,14-pentaaza- 6,9-methanonaphtho[1,8- ab]heptalen-12(13H)-one | 641 [M + H]⁺ |
| 28 | 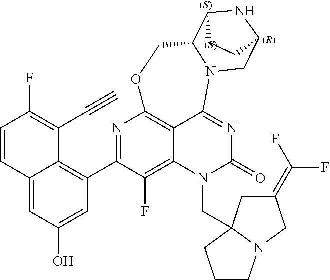 | (5aS,6S,9R)-13-((2- (difluoromethylene)tetrahydro- 1H-pyrrolizin-7a(5H)- yl)methyl)-2-(8-ethynyl-7- fluoro-3-hydroxynaphthalen-1- yl)-1-fluoro-5a,6,7,8,9,10- hexahydro-5H-4-oxa- 3,10a,11,13,14-pentaaza-6,9- methanonaphtho[1,8- ab]heptalen-12(13H)-one | 659 [M + H]⁺ |
| 29 | 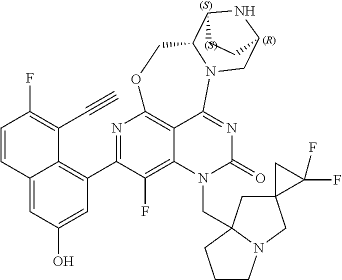 | (5aS,6S,9R)-13-((2,2- difluorodihydro-1′H,3′H- spiro[cyclopropane-1,2′- pyrrolizin]-7a′(5′H)-yl)methyl)- 2-(8-ethynyl-7-fluoro-3- hydroxynaphthalen-1-yl)-1- fluoro-5a,6,7,8,9,10-hexahydro- 5H-4-oxa-3,10a,11,13,14- pentaaza-6,9- methanonaphtho[1,8- ab]heptalen-12(13H)-one | 673 [M + H]⁺ |
| 30 | 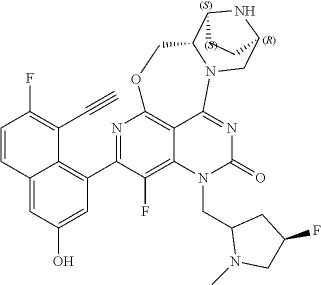 | (5aS,6S,9R)-2-(8-ethynyl-7- fluoro-3-hydroxynaphthalen-1- yl)-1-fluoro-13-(((4R)-4-fluoro- 1-methylpyrrolidin-2- yl)methyl)-5a,6,7,8,9,10- hexahydro-5H-4-oxa- 3,10a,11,13,14-pentaaza-6,9- methanonaphtho[1,8- ab]heptalen-12(13H)-one | 603 [M + H]⁺ |
| 31 | 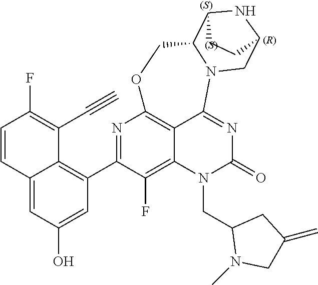 | (5aS,6S,9R)-2-(8-ethynyl-7- fluoro-3-hydroxynaphthalen-1- yl)-1-fluoro-13-((1-methyl-4- methylenepyrrolidin-2- yl)methyl)-5a,6,7,8,9,10- hexahydro-5H-4-oxa- 3,10a,11,13,14-pentaaza-6,9- methanonaphtho[1,8- ab]heptalen-12(13H)-one | 597 [M + H]⁺ |
| 32 | 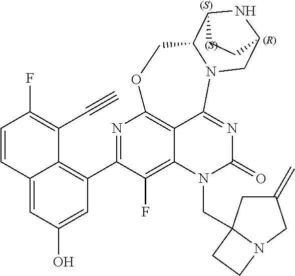 | (5aS,6S,9R)-2-(8-ethynyl-7- fluoro-3-hydroxynaphthalen-1- yl)-1-fluoro-13-((3-methylene-1- azabicyclo[3.2.0]heptan-5- yl)methyl)-5a,6,7,8,9,10- hexahydro-5H-4-oxa- 3,10a,11,13,14-pentaaza-6,9- methanonaphtho[1,8- ab]heptalen-12(13H)-one | 609 [M + H]⁺ |
| 33 | 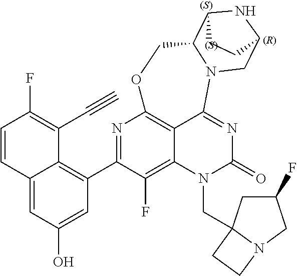 | (5aS,6S,9R)-2-(8-ethynyl-7- fluoro-3-hydroxynaphthalen-1- yl)-1-fluoro-13-(((3R)-3-fluoro- 1-azabicyclo[3.2.0]heptan-5- yl)methyl)-5a,6,7,8,9,10- hexahydro-5H-4-oxa- 3,10a,11,13,14-pentaaza-6,9- methanonaphtho[1,8- ab]heptalen-12(13H)-one | 615 [M + H]⁺ |
| 34 | 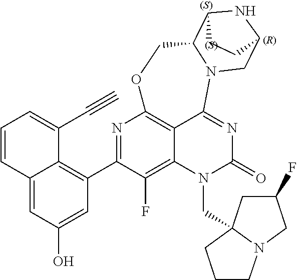 | (5aS,6S,9R)-2-(8-ethynyl-3- hydroxynaphthalen-1-yl)-1- fluoro-13-((2- methylenetetrahydro-1H- pyrrolizin-7a(5H)-yl)methyl)- 5a,6,7,8,9,10-hexahydro-5H-4- oxa-3,10a,11,13,14-pentaaza- 6,9-methanonaphtho[1,8- ab]heptalen-12(13H)-one | 611 [M + H]⁺ |
| 35 | 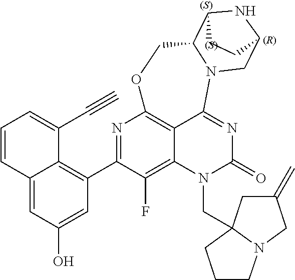 | (5aS,6S,9R)-13-((2,6- dimethylenetetrahydro-1H- pyrrolizin-7a(5H)-yl)methyl)-2- (8-ethynyl-3- hydroxynaphthalen-1-yl)-1- fluoro-5a,6,7,8,9,10-hexahydro- 5H-4-oxa-3,10a,11,13,14- pentaaza-6,9- methanonaphtho[1,8- ab]heptalen-12(13H)-one | 605 [M + H]⁺ |
| 36 | 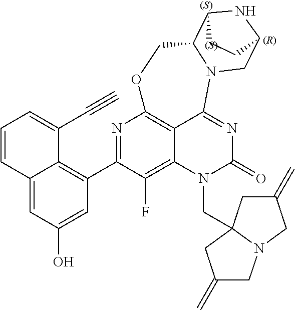 | (5aS,6S,9R)-2-(8-ethynyl-3- hydroxynaphthalen-1-yl)-1- fluoro-13-(((2R,7aS)-2- fluorotetrahydro-1H-pyrrolizin- 7a(5H)-yl)methyl)-5a,6,7,8,9,10- hexahydro-5H-4-oxa- 3,10a,11,13,14-pentaaza-6,9- methanonaphtho[1,8- ab]heptalen-12(13H)-one | 617 [M + H]⁺ |
| 37 | 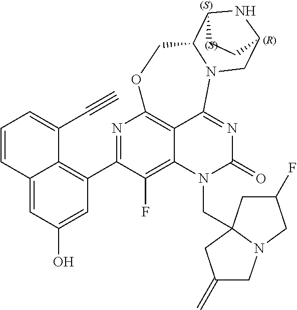 | (5aS,6S,9R)-2-(8-ethynyl-3- hydroxynaphthalen-1-yl)-1- fluoro-13-((2- (fluoromethylene)tetrahydro- 1H-pyrrolizin-7a(5H)- yl)methyl)-5a,6,7,8,9,10- hexahydro-5H-4-oxa- 3,10a,11,13,14-pentaaza-6,9- methanonaphtho[1,8- ab]heptalen-12(13H)-one | 623 [M + H]⁺ |
| 38 | 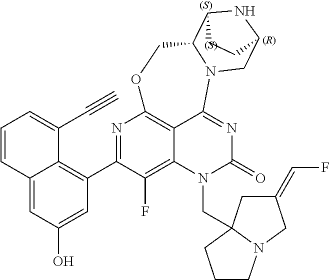 | (5aS,6S,9R)-2-(8-ethynyl-3- hydroxynaphthalen-1-yl)-1- fluoro-13-((2-fluoro-6- methylenetetrahydro-1H- pyrrolizin-7a(5H)-yl)methyl)- 5a,6,7,8,9,10-hexahydro-5H-4- oxa-3,10a,11,13,14-pentaaza- 6,9-methanonaphtho[1,8- ab]heptalen-12(13H)-one | 623 [M + H]⁺ |
| 39 | 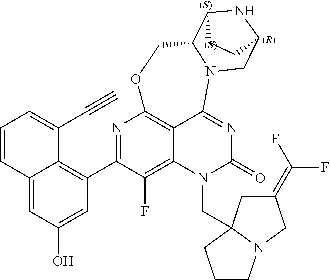 | (5aS,6S,9R)-13-((2- (difluoromethylene)tetrahydro- 1H-pyrrolizin-7a(5H)- yl)methyl)-2-(8-ethynyl-3- hydroxynaphthalen-1-yl)-1- fluoro-5a,6,7,8,9,10-hexahydro- 5H-4-oxa-3,10a,11,13,14- pentaaza-6,9- methanonaphtho[1,8- ab]heptalen-12(13H)-one | 641 [M + H]⁺ |
| 40 | 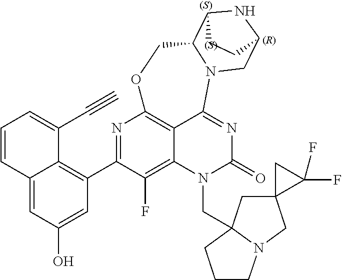 | (5aS,6S,9R)-13-((2,2- difluorodihydro-1′H,3′H- spiro[cyclopropane-1,2′- pyrrolizin]-7a′(5′H)-yl)methyl)- 2-(8-ethynyl-3- hydroxynaphthalen-1-yl)-1- fluoro-5a,6,7,8,9,10-hexahydro- 5H-4-oxa-3,10a,11,13,14- pentaaza-6,9- methanonaphtho[1,8- ab]heptalen-12(13H)-one | 655 [M + H]⁺ |
| 41 | 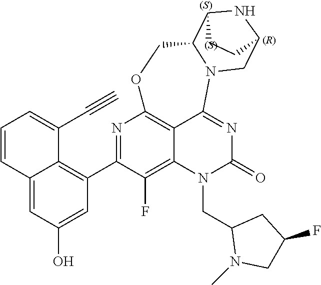 | (5aS,6S,9R)-2-(8-ethynyl-3- hydroxynaphthalen-1-yl)-1- fluoro-13-(((4R)-4-fluoro-1- methylpyrrolidin-2-yl)methyl)- 5a,6,7,8,9,10-hexahydro-5H-4- oxa-3,10a,11,13,14-pentaaza- 6,9-methanonaphtho[1,8- ab]heptalen-12(13H)-one | 585 [M + H]⁺ |
| 42 | 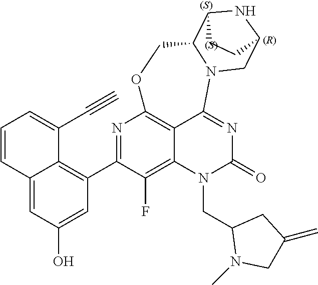 | (5aS,6S,9R)-2-(8-ethynyl-3- hydroxynaphthalen-1-yl)-1- fluoro-13-((1-methyl-4- methylenepyrrolidin-2- yl)methyl)-5a,6,7,8,9,10- hexahydro-5H-4-oxa- 3,10a,11,13,14-pentaaza-6,9- methanonaphtho[1,8- ab]heptalen-12(13H)-one | 579 [M + H]⁺ |
| 43 | 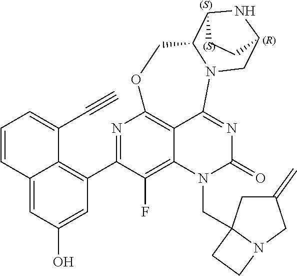 | (5aS,6S,9R)-2-(8-ethynyl-3- hydroxynaphthalen-1-yl)-1- fluoro-13-((3-methylene-1- azabicyclo[3.2.0]heptan-5- yl)methyl)-5a,6,7,8,9,10- hexahydro-5H-4-oxa- 3,10a,11,13,14-pentaaza-6,9- methanonaphtho[1,8- ab]heptalen-12(13H)-one | 591 [M + H]⁺ |
| 44 | 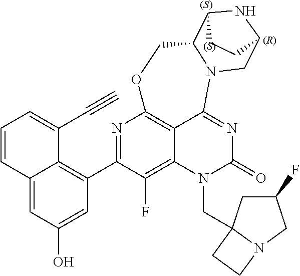 | (5aS,6S,9R)-2-(8-ethynyl-3- hydroxynaphthalen-1-yl)-1- fluoro-13-(((3R)-3-fluoro-1- azabicyclo[3.2.0]heptan-5- yl)methyl)-5a,6,7,8,9,10- hexahydro-5H-4-oxa- 3,10a,11,13,14-pentaaza-6,9- methanonaphtho[1,8- ab]heptalen-12(13H)-one | 597 [M + H]⁺ |
| 46a | 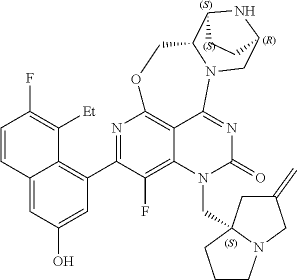 | (5aS,6S,9R)-2-(8-ethyl-7-fluoro- 3-hydroxynaphthalen-1-yl)-1- fluoro-13-(((S)-2- methylenetetrahydro-1H- pyrrolizin-7a(5H)-yl)methyl)- 5a,6,7,8,9,10-hexahydro-5H-4- oxa-3,10a,11,13,14-pentaaza- 6,9-methanonaphtho[1,8- ab]heptalen-12(13H)-one Isomer 1 (the first eluting isomer) | 627 [M + H]⁺ |
| 46b | 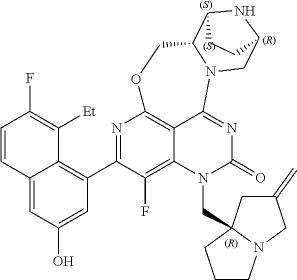 | (5aS,6S,9R)-2-(8-ethyl-7-fluoro- 3-hydroxynaphthalen-1-yl)-1- fluoro-13-(((R)-2- methylenetetrahydro-1H- pyrrolizin-7a(5H)-yl)methyl)- 5a,6,7,8,9,10-hexahydro-5H-4- oxa-3,10a,11,13,14-pentaaza- 6,9-methanonaphtho[1,8- ab]heptalen-12(13H)-one Isomer 2 (the second eluting isomer) | 627 [M + H]⁺ |
| 47 | 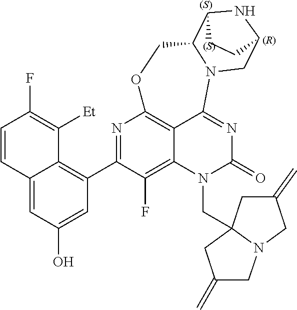 | (5aS,6S,9R)-2-(8-ethyl-7-fluoro- 3-hydroxynaphthalen-1-yl)-1- fluoro-13-(((2R,7aS)-2- fluorotetrahydro-1H-pyrrolizin- 7a(5H)-yl)methyl)-5a,6,7,8,9,10- hexahydro-5H-4-oxa- 3,10a,11,13,14-pentaaza-6,9- methanonaphtho[1,8- ab]heptalen-12(13H)-one | 639 [M + H]⁺ |
| 48 | 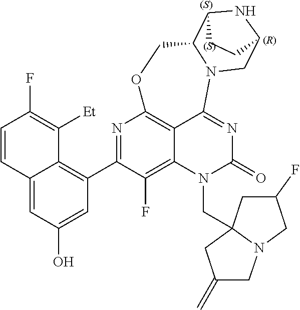 | (5aS,6S,9R)-2-(8-ethyl-7-fluoro- 3-hydroxynaphthalen-1-yl)-1- fluoro-13-((2- (fluoromethylene)tetrahydro- 1H-pyrrolizin-7a(5H)- yl)methyl)-5a,6,7,8,9,10- hexahydro-5H-4-oxa- 3,10a,11,13,14-pentaaza-6,9- methanonaphtho[1,8- ab]heptalen-12(13H)-one | 645 [M + H]⁺ |
| 49 | 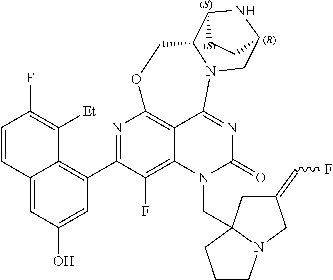 | (5aS,6S,9R)-2-(8-ethyl-7-fluoro- 3-hydroxynaphthalen-1-yl)-1- fluoro-13-((2-fluoro-6- methylenetetrahydro-1H- pyrrolizin-7a(5H)-yl)methyl)- 5a,6,7,8,9,10-hexahydro-5H-4- oxa-3,10a,11,13,14-pentaaza- 6,9-methanonaphtho[1,8- ab]heptalen-12(13H)-one | 645 [M + H]⁺ |
| 50 | 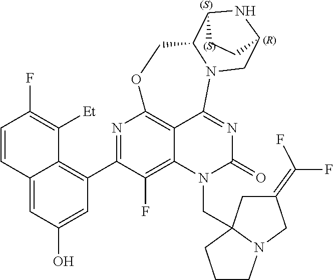 | (5aS,6S,9R)-13-((2- (difluoromethylene)tetrahydro- 1H-pyrrolizin-7a(5H)- yl)methyl)-2-(8-ethyl-7-fluoro- 3-hydroxynaphthalen-1-yl)-1- fluoro-5a,6,7,8,9,10-hexahydro- 5H-4-oxa-3,10a,11,13,14- pentaaza-6,9- methanonaphtho[1,8- ab]heptalen-12(13H)-one | 663 [M + H]⁺ |
| 51 | 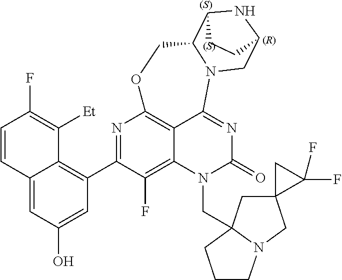 | (5aS,6S,9R)-13-((2,2- difluorodihydro-1′H,3′H- spiro[cyclopropane-1,2′- pyrrolizin]-7a′(5′H)-yl)methyl)- 2-(8-ethyl-7-fluoro-3- hydroxynaphthalen-1-yl)-1- fluoro-5a,6,7,8,9,10-hexahydro- 5H-4-oxa-3,10a,11,13,14- pentaaza-6,9- methanonaphtho[1,8- ab]heptalen-12(13H)-one | 677 [M + H]⁺ |
| 52 | 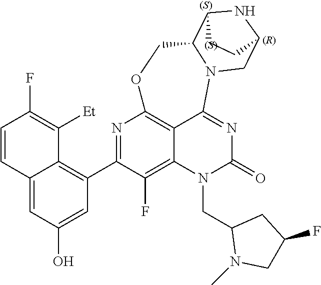 | (5aS,6S,9R)-2-(8-ethyl-7-fluoro- 3-hydroxynaphthalen-1-yl)-1- fluoro-13-(((4R)-4-fluoro-1- methylpyrrolidin-2-yl)methyl)- 5a,6,7,8,9,10-hexahydro-5H-4- oxa-3,10a,11,13,14-pentaaza- 6,9-methanonaphtho[1,8- ab]heptalen-12(13H)-one | 607 [M + H]⁺ |
| 53 | 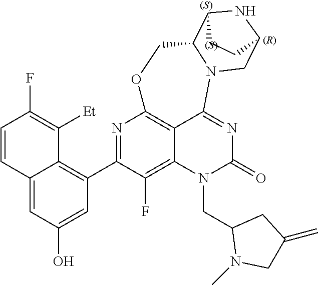 | (5aS,6S,9R)-2-(8-ethyl-7-fluoro- 3-hydroxynaphthalen-1-yl)-1- fluoro-13-((1-methyl-4- methylenepyrrolidin-2- yl)methyl)-5a,6,7,8,9,10- hexahydro-5H-4-oxa- 3,10a,11,13,14-pentaaza-6,9- methanonaphtho[1,8- ab]heptalen-12(13H)-one | 601 [M + H]⁺ |
| 54 | 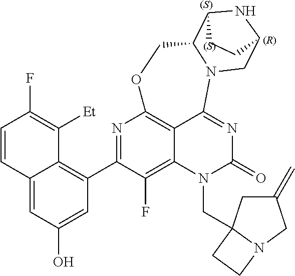 | (5aS,6S,9R)-2-(8-ethyl-7-fluoro- 3-hydroxynaphthalen-1-yl)-1- fluoro-13-((3-methylene-1- azabicyclo[3.2.0]heptan-5- yl)methyl)-5a,6,7,8,9,10- hexahydro-5H-4-oxa- 3,10a,11,13,14-pentaaza-6,9- methanonaphtho[1,8- ab]heptalen-12(13H)-one | 613 [M + H]⁺ |
| 55 | 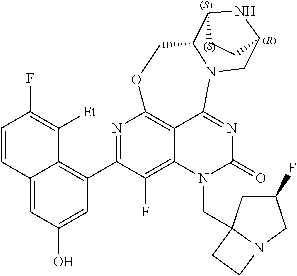 | (5aS,6S,9R)-2-(8-ethyl-7-fluoro- 3-hydroxynaphthalen-1-yl)-1- fluoro-13-(((3R)-3-fluoro-1- azabicyclo[3.2.0]heptan-5- yl)methyl)-5a,6,7,8,9,10- hexahydro-5H-4-oxa- 3,10a,11,13,14-pentaaza-6,9- methanonaphtho[1,8- ab]heptalen-12(13H)-one | 619 [M + H]⁺ |
| 56 | 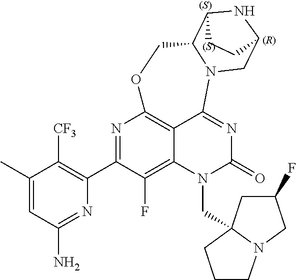 | (5aS,6S,9R)-2-(6-amino-4- methyl-3- (trifluoromethyl)pyridin-2-yl)-1- fluoro-13-((2- methylenetetrahydro-1H- pyrrolizin-7a(5H)-yl)methyl)- 5a,6,7,8,9,10-hexahydro-5H-4- oxa-3,10a,11,13,14-pentaaza- 6,9-methanonaphtho[1,8- ab]heptalen-12(13H)-one | 619 [M + H]⁺ |
| 57 | 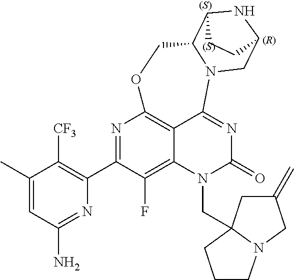 | (5aS,6S,9R)-2-(6-amino-4- methyl-3- (trifluoromethyl)pyridin-2-yl)- 13-((2,6-dimethylenetetrahydro- 1H-pyrrolizin-7a(5H)- yl)methyl)-1-fluoro- 5a,6,7,8,9,10-hexahydro-5H-4- oxa-3,10a,11,13,14-pentaaza- 6,9-methanonaphtho[1,8- ab]heptalen-12(13H)-one | 613 [M + H]⁺ |
| 58 | 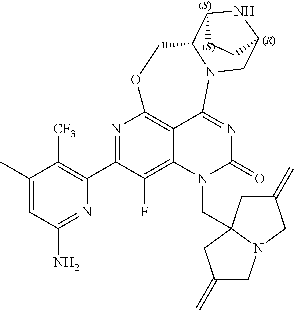 | (5aS,6S,9R)-2-(6-amino-4- methyl-3- (trifluoromethyl)pyridin-2-yl)-1- fluoro-13-(((2R,7aS)-2- fluorotetrahydro-1H-pyrrolizin- 7a(5H)-yl)methyl)-5a,6,7,8,9,10- hexahydro-5H-4-oxa- 3,10a,11,13,14-pentaaza-6,9- methanonaphtho[1,8- ab]heptalen-12(13H)-one | 625 [M + H]⁺ |
| 59 | 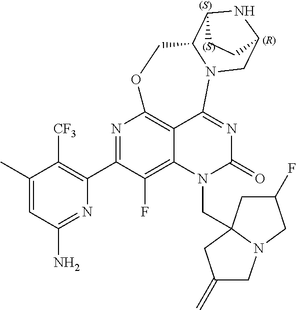 | (5aS,6S,9R)-2-(6-amino-4- methyl-3- (trifluoromethyl)pyridin-2-yl)-1- fluoro-13-((2- (fluoromethylene)tetrahydro- 1H-pyrrolizin-7a(5H)- yl)methyl)-5a,6,7,8,9,10- hexahydro-5H-4-oxa- 3,10a,11,13,14-pentaaza-6,9- methanonaphtho[1,8- ab]heptalen-12(13H)-one | 631 [M + H]⁺ |
| 60 | 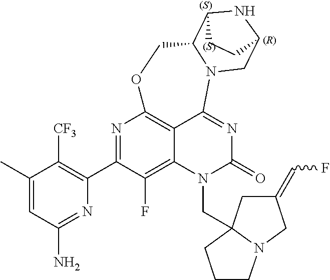 | (5aS,6S,9R)-2-(6-amino-4- methyl-3- (trifluoromethyl)pyridin-2-yl)-1- fluoro-13-((2-fluoro-6- methylenetetrahydro-1H- pyrrolizin-7a(5H)-yl)methyl)- 5a,6,7,8,9,10-hexahydro-5H-4- oxa-3,10a,11,13,14-pentaaza- 6,9-methanonaphtho[1,8- ab]heptalen-12(13H)-one | 631 [M + H]⁺ |
| 61 | 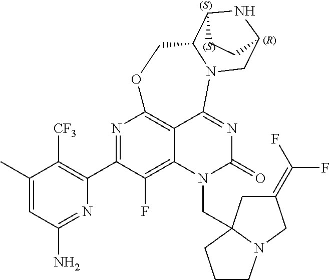 | (5aS,6S,9R)-2-(6-amino-4- methyl-3- (trifluoromethyl)pyridin-2-yl)- 13-((2- (difluoromethylene)tetrahydro- 1H-pyrrolizin-7a(5H)- yl)methyl)-1-fluoro- 5a,6,7,8,9,10-hexahydro-5H-4- oxa-3,10a,11,13,14-pentaaza- 6,9-methanonaphtho[1,8- ab]heptalen-12(13H)-one | 649 [M + H]⁺ |
| 62 | 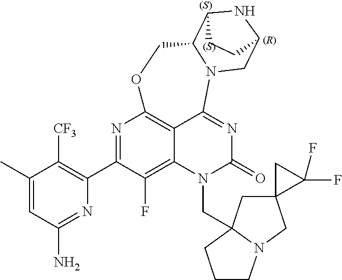 | (5aS,6S,9R)-2-(6-amino-4- methyl-3- (trifluoromethyl)pyridin-2-yl)- 13-((2,2-difluorodihydro- 1′H,3′H-spiro[cyclopropane-1,2′- pyrrolizin]-7a′(5′H)-yl)methyl)- 1-fluoro-5a,6,7,8,9,10- hexahydro-5H-4-oxa- 3,10a,11,13,14-pentaaza-6,9- methanonaphtho[1,8- ab]heptalen-12(13H)-one | 663 [M + H]⁺ |
| 63 | 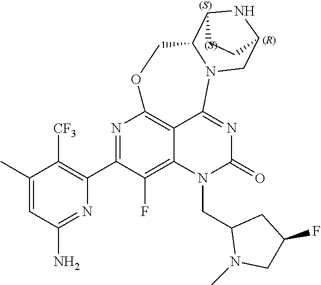 | (5aS,6S,9R)-2-(6-amino-4- methyl-3- (trifluoromethyl)pyridin-2-yl)-1- fluoro-13-(((4R)-4-fluoro-1- methylpyrrolidin-2-yl)methyl)- 5a,6,7,8,9,10-hexahydro-5H-4- oxa-3,10a,11,13,14-pentaaza- 6,9-methanonaphtho[1,8- ab]heptalen-12(13H)-one | 593 [M + H]⁺ |
| 64 | 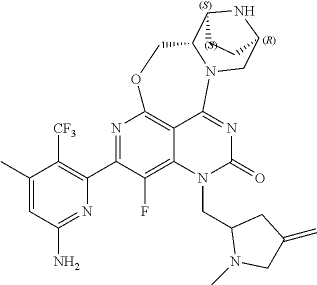 | (5aS,6S,9R)-2-(6-amino-4- methyl-3- (trifluoromethyl)pyridin-2-yl)-1- fluoro-13-((1-methyl-4- methylenepyrrolidin-2- yl)methyl)-5a,6,7,8,9,10- hexahydro-5H-4-oxa- 3,10a,11,13,14-pentaaza-6,9- methanonaphtho[1,8- ab]heptalen-12(13H)-one | 587 [M + H]⁺ |
| 65 | 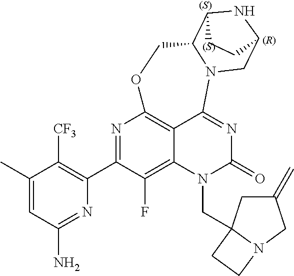 | (5aS,6S,9R)-2-(6-amino-4- methyl-3- (trifluoromethyl)pyridin-2-yl)-1- fluoro-13-(((3R)-3-fluoro-1- azabicyclo[3.2.0]heptan-5- yl)methyl)-5a,6,7,8,9,10- hexahydro-5H-4-oxa- 3,10a,11,13,14-pentaaza-6,9- methanonaphtho[1,8- ab]heptalen-12(13H)-one | 599 [M + H]⁺ |
| 66 | 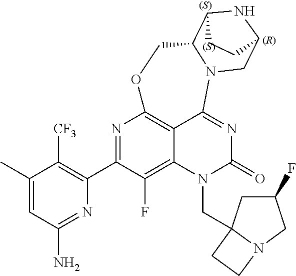 | (5aS,6S,9R)-2-(6-amino-4- methyl-3- (trifluoromethyl)pyridin-2-yl)-1- fluoro-13-((3-methylene-1- azabicyclo[3.2.0]heptan-5- yl)methyl)-5a,6,7,8,9,10- hexahydro-5H-4-oxa- 3,10a,11,13,14-pentaaza-6,9- methanonaphtho[1,8- ab]heptalen-12(13H)-one | 605 [M + H]⁺ |
| 67 | 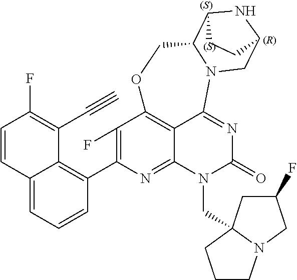 | (5aS,6S,9R)-2-(8-ethynyl-7- fluoronaphthalen-1-yl)-3-fluoro- 13-((2-methylenetetrahydro-1H- pyrrolizin-7a(5H)-yl)methyl)- 5a,6,7,8,9,10-hexahydro-5H-4- oxa-1,10a,11,13,14-pentaaza- 6,9-methanonaphtho[1,8- ab]heptalen-12(13H)-one | 613 [M + H]⁺ |
| 68 | 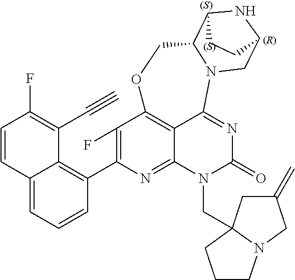 | (5aS,6S,9R)-13-((2,6- dimethylenetetrahydro-1H- pyrrolizin-7a(5H)-yl)methyl)-2- (8-ethynyl-7-fluoronaphthalen- 1-yl)-3-fluoro-5a,6,7,8,9,10- hexahydro-5H-4-oxa- 1,10a,11,13,14-pentaaza-6,9- methanonaphtho[1,8- ab]heptalen-12(13H)-one | 607 [M + H]⁺ |
| 69 | 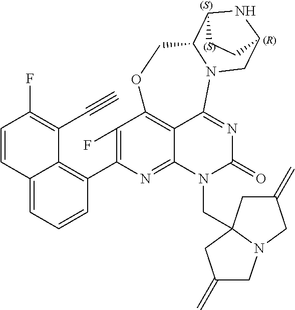 | (5aS,6S,9R)-2-(8-ethynyl-7- fluoronaphthalen-1-yl)-3-fluoro- 13-(((2R,7aS)-2- fluorotetrahydro-1 H-pyrrolizin- 7a(5H)-yl)methyl)-5a,6,7,8,9,10- hexahydro-5H-4-oxa- 1,10a,11,13,14-pentaaza-6,9- methanonaphtho[1,8- ab]heptalen-12(13H)-one | 619 [M + H]⁺ |
| 70 | 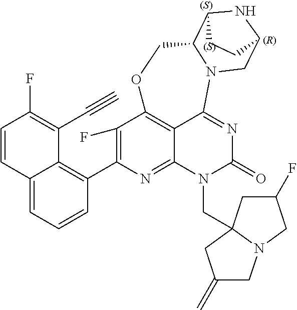 | (5aS,6S,9R)-2-(8-ethynyl-7- fluoronaphthalen-1-yl)-3-fluoro- 13-((2- (fluoromethylene)tetrahydro- 1H-pyrrolizin-7a(5H)- yl)methyl)-5a,6,7,8,9,10- hexahydro-5H-4-oxa- 1,10a,11,13,14-pentaaza-6,9- methanonaphtho[1,8- ab]heptalen-12(13H)-one | 625 [M + H]⁺ |
| 71 | 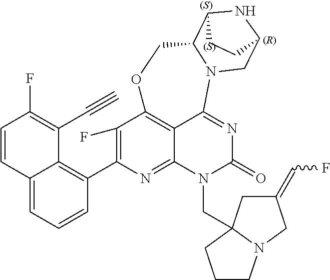 | (5aS,6S,9R)-2-(8-ethynyl-7- fluoronaphthalen-1-yl)-3-fluoro- 13-((2-fluoro-6- methylenetetrahydro-1H- pyrrolizin-7a(5H)-yl)methyl)- 5a,6,7,8,9,10-hexahydro-5H-4- oxa-1,10a,11,13,14-pentaaza- 6,9-methanonaphtho[1,8- ab]heptalen-12(13H)-one | 625 [M + H]⁺ |
| 72 | 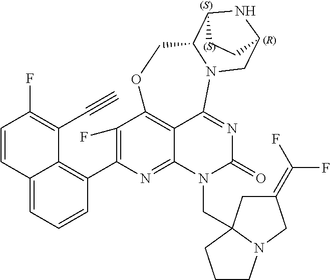 | (5aS,6S,9R)-13-((2- (difluoromethylene)tetrahydro- 1H-pyrrolizin-7a(5H)- yl)methyl)-2-(8-ethynyl-7- fluoronaphthalen-1-yl)-3-fluoro- 5a,6,7,8,9,10-hexahydro-5H-4- oxa-1,10a,11,13,14-pentaaza- 6,9-methanonaphtho[1,8- ab]heptalen-12(13H)-one | 643 [M + H]⁺ |
| 73 | 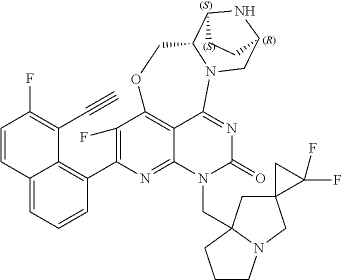 | (5aS,6S,9R)-13-((2,2- difluorodihydro-1′H,3′H- spiro[cyclopropane-1,2′- pyrrolizin]-7a′(5′H)-yl)methyl)- 2-(8-ethynyl-7- fluoronaphthalen-1-yl)-3-fluoro- 5a,6,7,8,9,10-hexahydro-5H-4- oxa-1,10a,11,13,14-pentaaza- 6,9-methanonaphtho[1,8- ab]heptalen-12(13H)-one | 657 [M + H]⁺ |
| 74 | 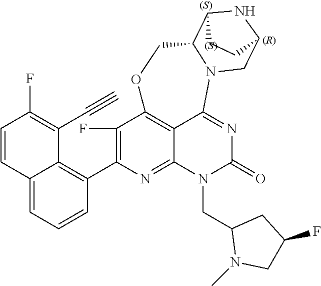 | (5aS,6S,9R)-2-(8-ethynyl-7- fluoronaphthalen-1-yl)-3-fluoro- 13-(((4R)-4-fluoro-1- methylpyrrolidin-2-yl)methyl)- 5a,6,7,8,9,10-hexahydro-5H-4- oxa-1,10a,11,13,14-pentaaza- 6,9-methanonaphtho[1,8- ab]heptalen-12(13H)-one | 587 [M + H]⁺ |
| 75 | 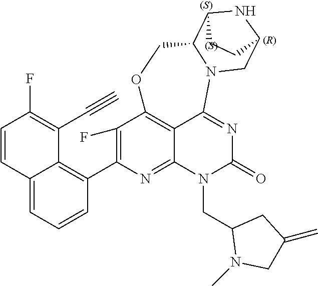 | (5aS,6S,9R)-2-(8-ethynyl-7- fluoronaphthalen-1-yl)-3-fluoro- 13-((1-methyl-4- methylenepyrrolidin-2- yl)methyl)-5a,6,7,8,9,10- hexahydro-5H-4-oxa- 1,10a,11,13,14-pentaaza-6,9- methanonaphtho[1,8- ab]heptalen-12(13H)-one | 581 [M + H]⁺ |
| 76 | 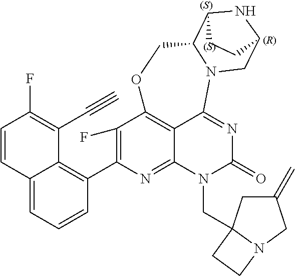 | (5aS,6S,9R)-2-(8-ethynyl-7- fluoronaphthalen-1-yl)-3-fluoro- 13-((3-methylene-1- azabicyclo[3.2.0]heptan-5- yl)methyl)-5a,6,7,8,9,10- hexahydro-5H-4-oxa- 1,10a,11,13,14-pentaaza-6,9- methanonaphtho[1,8- ab]heptalen-12(13H)-one | 593 [M + H]⁺ |
| 77 | 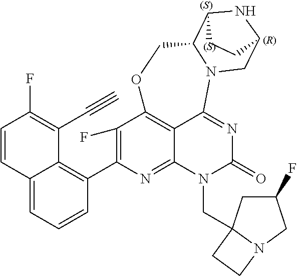 | (5aS,6S,9R)-2-(8-ethynyl-7- fluoronaphthalen-1-yl)-3-fluoro- 13-(((3R)-3-fluoro-1- azabicyclo[3.2.0]heptan-5- yl)methyl)-5a,6,7,8,9,10- hexahydro-5H-4-oxa- 1,10a,11,13,14-pentaaza-6,9- methanonaphtho[1,8- ab]heptalen-12(13H)-one | 599 [M + H]⁺ |
| 78 | 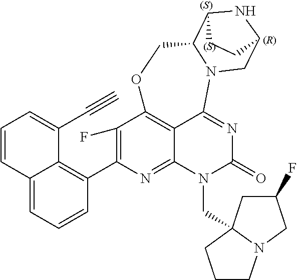 | (5aS,6S,9R)-2-(8- ethynylnaphthalen-1-yl)-3- fluoro-13-((2- methylenetetrahydro-1H- pyrrolizin-7a(5H)-yl)methyl)- 5a,6,7,8,9,10-hexahydro-5H-4- oxa-1,10a,11,13,14-pentaaza- 6,9-methanonaphtho[1,8- ab]heptalen-12(13H)-one | 595 [M + H]⁺ |
| 79 | 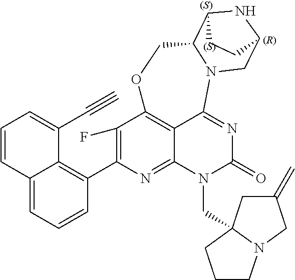 | (5aS,6S,9R)-13-((2,6- dimethylenetetrahydro-1H- pyrrolizin-7a(5H)-yl)methyl)-2- (8-ethynylnaphthalen-1-yl)-3- fluoro-5a,6,7,8,9,10-hexahydro- 5H-4-oxa-1,10a,11,13,14- pentaaza-6,9- methanonaphtho[1,8- ab]heptalen-12(13H)-one | 589 [M + H]⁺ |
| 80 | 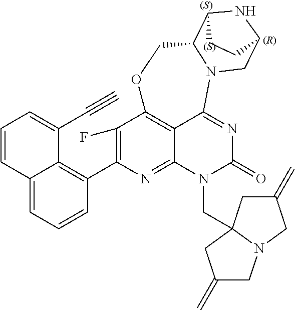 | (5aS,6S,9R)-2-(8- ethynylnaphthalen-1-yl)-3- fluoro-13-(((2R,7aS)-2- fluorotetrahydro-1H-pyrrolizin- 7a(5H)-yl)methyl)-5a,6,7,8,9,10- hexahydro-5H-4-oxa- 1,10a,11,13,14-pentaaza-6,9- methanonaphtho[1,8- ab]heptalen-12(13H)-one | 601 [M + H]⁺ |
| 81 | 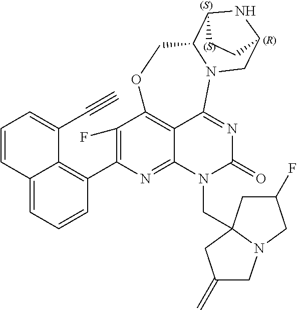 | (5aS,6S,9R)-2-(8- ethynylnaphthalen-1-yl)-3- fluoro-13-((2- (fluoromethylene)tetrahydro- 1H-pyrrolizin-7a(5H)- yl)methyl)-5a,6,7,8,9,10- hexahydro-5H-4-oxa- 1,10a,11,13,14-pentaaza-6,9- methanonaphtho[1,8- ab]heptalen-12(13H)-one | 607 [M + H]⁺ |
| 82 | 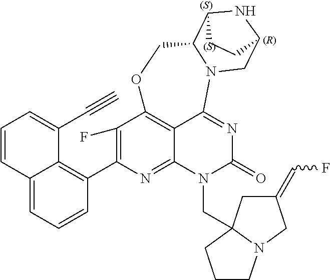 | (5aS,6S,9R)-2-(8- ethynylnaphthalen-1-yl)-3- fluoro-13-((2-fluoro-6- methylenetetrahydro-1H- pyrrolizin-7a(5H)-yl)methyl)- 5a,6,7,8,9,10-hexahydro-5H-4- oxa-1,10a,11,13,14-pentaaza- 6,9-methanonaphtho[1,8- ab]heptalen-12(13H)-one | 607 [M + H]⁺ |
| 83 | 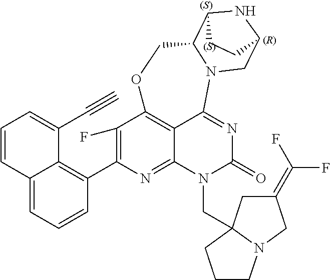 | (5aS,6S,9R)-13-((2- (difluoromethylene)tetrahydro- 1H-pyrrolizin-7a(5H)- yl)methyl)-2-(8- ethynylnaphthalen-1-yl)-3- fluoro-5a,6,7,8,9,10-hexahydro- 5H-4-oxa-1,10a,11,13,14- pentaaza-6,9- methanonaphtho[1,8- ab]heptalen-12(13H)-one | 625 [M + H]⁺ |
| 84 | 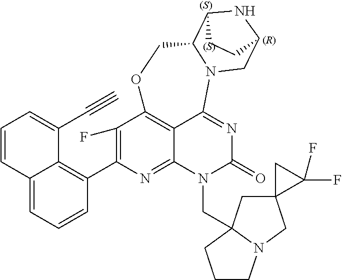 | (5aS,6S,9R)-13-((2,2- difluorodihydro-1′H,3′H- spiro[cyclopropane-1,2′- pyrrolizin]-7a′(5′H)-yl)methyl)- 2-(8-ethynylnaphthalen-1-yl)-3- fluoro-5a,6,7,8,9,10-hexahydro- 5H-4-oxa-1,10a,11,13,14- pentaaza-6,9- methanonaphtho[1,8- ab]heptalen-12(13H)-one | 639 [M + H]⁺ |
| 85 | 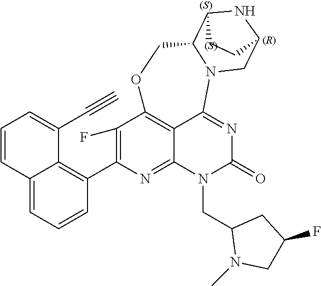 | (5aS,6S,9R)-2-(8- ethynylnaphthalen-1-yl)-3- fluoro-13-(((4R)-4-fluoro-1- methylpyrrolidin-2-yl)methyl)- 5a,6,7,8,9,10-hexahydro-5H-4- oxa-1,10a,11,13,14-pentaaza- 6,9-methanonaphtho[1,8- ab]heptalen-12(13H)-one | 569 [M + H]⁺ |
| 86 | 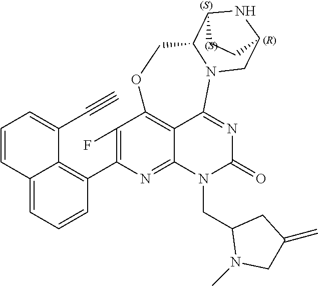 | (5aS,6S,9R)-2-(8- ethynylnaphthalen-1-yl)-3- fluoro-13-((1-methyl-4- methylenepyrrolidin-2- yl)methyl)-5a,6,7,8,9,10- hexahydro-5H-4-oxa- 1,10a,11,13,14-pentaaza-6,9- methanonaphtho[1,8- ab]heptalen-12(13H)-one | 563 [M + H]⁺ |
| 87 | 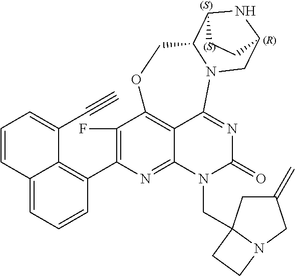 | (5aS,6S,9R)-2-(8- ethynylnaphthalen-1-yl)-3- fluoro-13-((3-methylene-1- azabicyclo[3.2.0]heptan-5- yl)methyl)-5a,6,7,8,9,10- hexahydro-5H-4-oxa- 1,10a,11,13,14-pentaaza-6,9- methanonaphtho[1,8- ab]heptalen-12(13H)-one | 575 [M + H]⁺ |
| 88 | 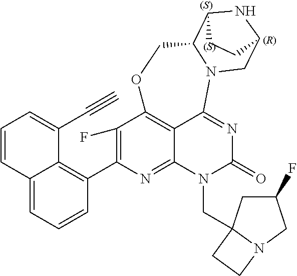 | (5aS,6S,9R)-2-(8- ethynylnaphthalen-1-yl)-3- fluoro-13-(((3R)-3-fluoro-1- azabicyclo[3.2.0]heptan-5- yl)methyl)-5a,6,7,8,9,10- hexahydro-5H-4-oxa- 1,10a,11,13,14-pentaaza-6,9- methanonaphtho[1,8- ab]heptalen-12(13H)-one | 581 [M + H]⁺ |
| 89 | 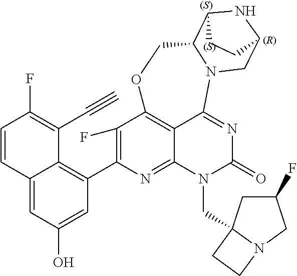 | (5aS,6S,9R)-2-(8-ethynyl-7- fluoro-3-hydroxynaphthalen-1- yl)-3-fluoro-13-((2- methylenetetrahydro-1H- pyrrolizin-7a(5H)-yl)methyl)- 5a,6,7,8,9,10-hexahydro-5H-4- oxa-1,10a,11,13,14-pentaaza- 6,9-methanonaphtho[1,8- ab]heptalen-12(13H)-one | 629 [M + H]⁺ |
| 90 | 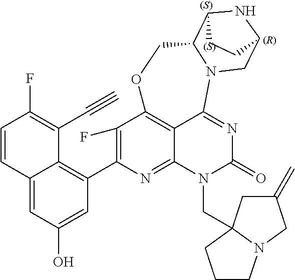 | (5aS,6S,9R)-13-((2,6- dimethylenetetrahydro-1H- pyrrolizin-7a(5H)-yl)methyl)-2- (8-ethynyl-7-fluoro-3- hydroxynaphthalen-1-yl)-3- fluoro-5a,6,7,8,9,10-hexahydro- 5H-4-oxa-1,10a,11,13,14- pentaaza-6,9- methanonaphtho[1,8- ab]heptalen-12(13H)-one | 623 [M + H]⁺ |
| 91 | 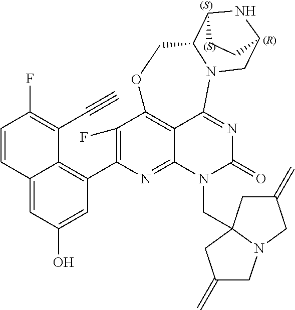 | (5aS,6S,9R)-2-(8-ethynyl-7- fluoro-3-hydroxynaphthalen-1- yl)-3-fluoro-13-(((2R,7aS)-2- fluorotetrahydro-1H-pyrrolizin- 7a(5H)-yl)methyl)-5a,6,7,8,9,10- hexahydro-5H-4-oxa- 1,10a,11,13,14-pentaaza-6,9- methanonaphtho[1,8- ab]heptalen-12(13H)-one | 635 [M + H]⁺ |
| 92 | 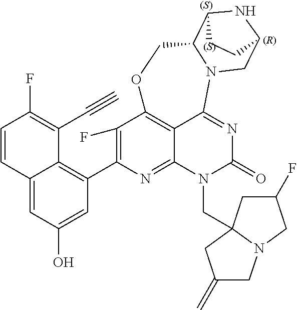 | (5aS,6S,9R)-2-(8-ethynyl-7- fluoro-3-hydroxynaphthalen-1- yl)-3-fluoro-13-((2- (fluoromethylene)tetrahydro- 1H-pyrrolizin-7a(5H)- yl)methyl)-5a,6,7,8,9,10- hexahydro-5H-4-oxa- 1,10a,11,13,14-pentaaza-6,9- methanonaphtho[1,8- ab]heptalen-12(13H)-one | 641 [M + H]⁺ |
| 93 | 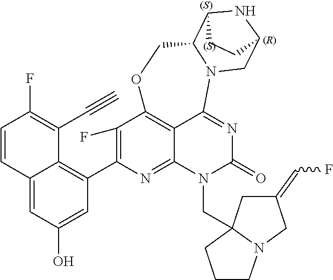 | (5aS,6S,9R)-2-(8-ethynyl-7- fluoro-3-hydroxynaphthalen-1 - yl)-3-fluoro-13-((2-fluoro-6- methylenetetrahydro-1H- pyrrolizin-7a(5H)-yl)methyl)- 5a,6,7,8,9,10-hexahydro-5H-4- oxa-1,10a,11,13,14-pentaaza- 6,9-methanonaphtho[1,8- ab]heptalen-12(13H)-one | 641 [M + H]⁺ |
| 94 | 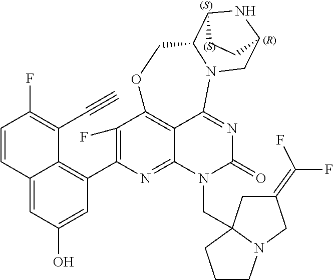 | (5aS,6S,9R)-13-((2- (difluoromethylene)tetrahydro- 1H-pyrrolizin-7a(5H)- yl)methyl)-2-(8-ethynyl-7- fluoro-3-hydroxynaphthalen-1- yl)-3-fluoro-5a,6,7,8,9,10- hexahydro-5H-4-oxa- 1,10a,11,13,14-pentaaza-6,9- methanonaphtho[1,8- ab]heptalen-12(13H)-one | 659 [M + H]⁺ |
| 95 | 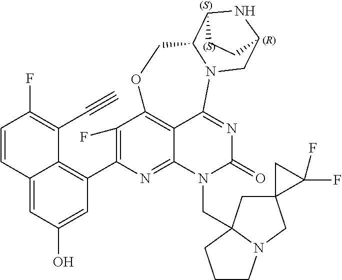 | (5aS,6S,9R)-13-((2,2- difluorodihydro-1′H,3′H- spiro[cyclopropane-1,2′- pyrrolizin]-7a′(5′H)-yl)methyl)- 2-(8-ethynyl-7-fluoro-3- hydroxynaphthalen-1-yl)-3- fluoro-5a,6,7,8,9,10-hexahydro- 5H-4-oxa-1,10a,11,13,14- pentaaza-6,9- methanonaphtho[1,8- ab]heptalen-12(13H)-one | 673 [M + H]⁺ |
| 96 | 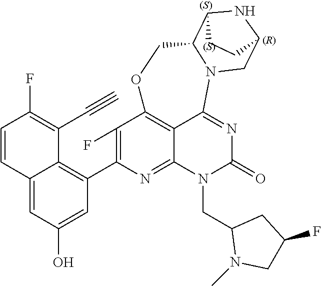 | (5aS,6S,9R)-2-(8-ethynyl-7- fluoro-3-hydroxynaphthalen-1- yl)-3-fluoro-13-(((4R)-4-fluoro- 1-methylpyrrolidin-2- yl)methyl)-5a,6,7,8,9,10- hexahydro-5H-4-oxa- 1,10a,11,13,14-pentaaza-6,9- methanonaphtho[1,8- ab]heptalen-12(13H)-one | 603 [M + H]⁺ |
| 97 | 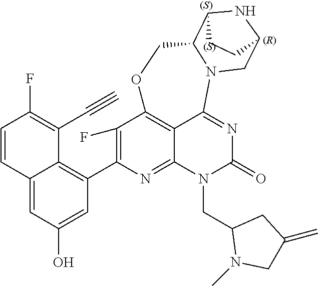 | (5aS,6S,9R)-2-(8-ethynyl-7- fluoro-3-hydroxynaphthalen-1- yl)-3-fluoro-13-((1-methyl-4- methylenepyrrolidin-2- yl)methyl)-5a,6,7,8,9,10- hexahydro-5H-4-oxa- 1,10a,11,13,14-pentaaza-6,9- methanonaphtho[1,8- ab]heptalen-12(13H)-one | 597 [M + H]⁺ |
| 98 | 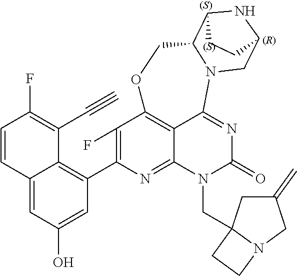 | (5aS,6S,9R)-2-(8-ethynyl-7- fluoro-3-hydroxynaphthalen-1- yl)-3-fluoro-13-((3-methylene-1- azabicyclo[3.2.0]heptan-5- yl)methyl)-5a,6,7,8,9,10- hexahydro-5H-4-oxa- 1,10a,11,13,14-pentaaza-6,9- methanonaphtho[1,8- ab]heptalen-12(13H)-one | 609 [M + H]⁺ |
| 99 | 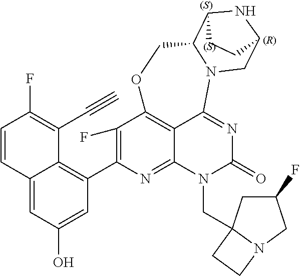 | (5aS,6S,9R)-2-(8-ethynyl-7- fluoro-3-hydroxynaphthalen-1 - yl)-3-fluoro-13-(((3R)-3-fluoro- 1-azabicyclo[3.2.0]heptan-5- yl)methyl)-5a,6,7,8,9,10- hexahydro-5H-4-oxa- 1,10a,11,13,14-pentaaza-6,9- methanonaphtho[1,8- ab]heptalen-12(13H)-one | 615 [M + H]⁺ |
| 100 | 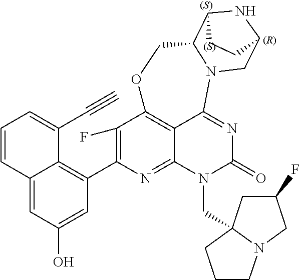 | (5aS,6S,9R)-2-(8-ethynyl-3- hydroxynaphthalen-1-yl)-3- fluoro-13-(((2R,7aS)-2- fluorotetrahydro-1H-pyrrolizin- 7a(5H)-yl)methyl)-5a,6,7,8,9,10- hexahydro-5H-4-oxa- 1,10a,11,13,14-pentaaza-6,9- methanonaphtho[1,8- ab]heptalen-12(13H)-one | 611 [M + H]⁺ |
| 101 | 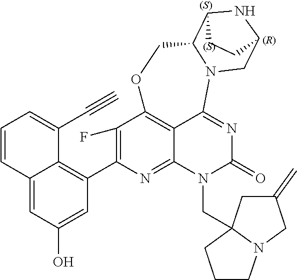 | (5aS,6S,9R)-2-(8-ethynyl-3- hydroxynaphthalen-1-yl)-3- fluoro-13-((2- methylenetetrahydro-1H- pyrrolizin-7a(5H)-yl)methyl)- 5a,6,7,8,9,10-hexahydro-5H-4- oxa-1,10a,11,13,14-pentaaza- 6,9-methanonaphtho[1,8- ab]heptalen-12(13H)-one | 605 [M + H]⁺ |
| 102 | 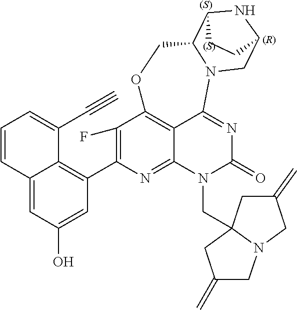 | (5aS,6S,9R)-13-((2,6- dimethylenetetrahydro-1H- pyrrolizin-7a(5H)-yl)methyl)-2- (8-ethynyl-3- hydroxynaphthalen-1-yl)-3- fluoro-5a,6,7,8,9,10-hexahydro- 5H-4-oxa-1,10a,11,13,14- pentaaza-6,9- methanonaphtho[1,8- ab]heptalen-12(13H)-one | 617 [M + H]⁺ |
| 103 | 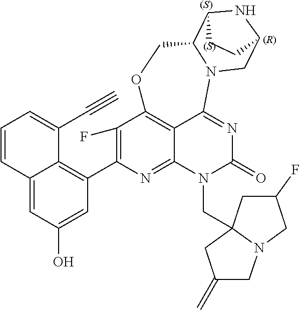 | (5aS,6S,9R)-2-(8-ethynyl-3- hydroxynaphthalen-1-yl)-3- fluoro-13-((2- (fluoromethylene)tetrahydro- 1H-pyrrolizin-7a(5H)- yl)methyl)-5a,6,7,8,9,10- hexahydro-5H-4-oxa- 1,10a,11,13,14-pentaaza-6,9- methanonaphtho[1,8- ab]heptalen-12(13H)-one | 623 [M + H]⁺ |
| 104 | 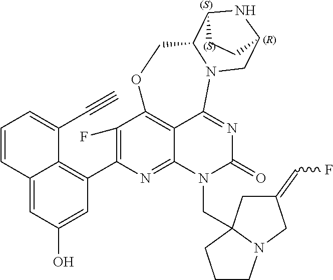 | (5aS,6S,9R)-2-(8-ethynyl-3- hydroxynaphthalen-1-yl)-3- fluoro-13-((2-fluoro-6- methylenetetrahydro-1H- pyrrolizin-7a(5H)-yl)methyl)- 5a,6,7,8,9,10-hexahydro-5H-4- oxa-1,10a,11,13,14-pentaaza- 6,9-methanonaphtho[1,8- ab]heptalen-12(13H)-one | 623 [M + H]⁺ |
| 105 | 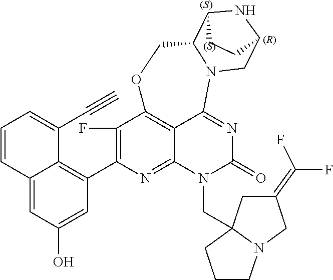 | (5aS,6S,9R)-13-((2- (difluoromethylene)tetrahydro- 1H-pyrrolizin-7a(5H)- yl)methyl)-2-(8-ethynyl-3- hydroxynaphthalen-1-yl)-3- fluoro-5a,6,7,8,9,10-hexahydro- 5H-4-oxa-1,10a,11,13,14- pentaaza-6,9- methanonaphtho[1,8- ab]heptalen-12(13H)-one | 641 [M + H]⁺ |
| 106 | 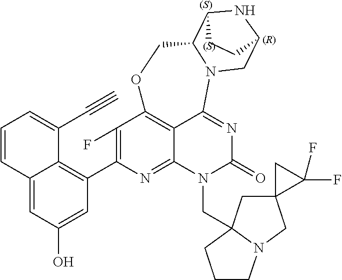 | (5aS,6S,9R)-13-((2,2- difluorodihydro-1′H,3′H- spiro[cyclopropane-1,2′- pyrrolizin]-7a′(5′H)-yl)methyl)- 2-(8-ethynyl-3- hydroxynaphthalen-1-yl)-3- fluoro-5a,6,7,8,9,10-hexahydro- 5H-4-oxa-1,10a,11,13,14- pentaaza-6,9- methanonaphtho[1,8- ab]heptalen-12(13H)-one | 655 [M + H]⁺ |
| 107 | 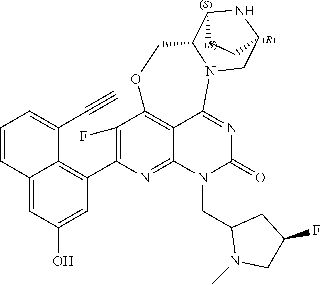 | (5aS,6S,9R)-2-(8-ethynyl-3- hydroxynaphthalen-1-yl)-3- fluoro-13-(((4R)-4-fluoro-1- methylpyrrolidin-2-yl)methyl)- 5a,6,7,8,9,10-hexahydro-5H-4- oxa-1,10a,11,13,14-pentaaza- 6,9-methanonaphtho[1,8- ab]heptalen-12(13H)-one | 585 [M + H]⁺ |
| 108 | 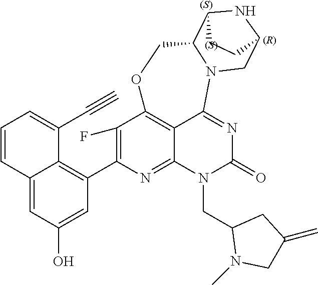 | (5aS,6S,9R)-2-(8-ethynyl-3- hydroxynaphthalen-1-yl)-3- fluoro-13-((1-methyl-4- methylenepyrrolidin-2- yl)methyl)-5a,6,7,8,9,10- hexahydro-5H-4-oxa- 1,10a,11,13,14-pentaaza-6,9- methanonaphtho[1,8- ab]heptalen-12(13H)-one | 579 [M + H]⁺ |
| 109 | 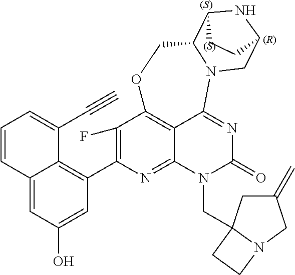 | (5aS,6S,9R)-2-(8-ethynyl-3- hydroxynaphthalen-1-yl)-3- fluoro-13-((3-methylene-1- azabicyclo[3.2.0]heptan-5- yl)methyl)-5a,6,7,8,9,10- hexahydro-5H-4-oxa- 1,10a,11,13,14-pentaaza-6,9- methanonaphtho[1,8- ab]heptalen-12(13H)-one | 591 [M + H]⁺ |
| 110 | 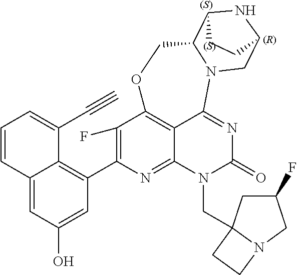 | (5aS,6S,9R)-2-(8-ethynyl-3- hydroxynaphthalen-1-yl)-3- fluoro-13-(((3R)-3-fluoro-1- azabicyclo[3.2.0]heptan-5- yl)methyl)-5a,6,7,8,9,10- hexahydro-5H-4-oxa- 1,10a,11,13,14-pentaaza-6,9- methanonaphtho[1,8- ab]heptalen-12(13H)-one | 597 [M + H]⁺ |
| 111 | 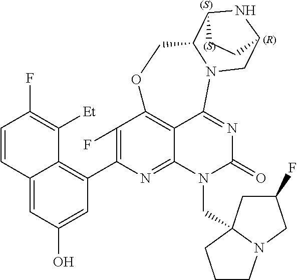 | (5aS,6S,9R)-2-(8-ethyl-7-fluoro- 3-hydroxynaphthalen-1-yl)-3- fluoro-13-((2- methylenetetrahydro-1H- pyrrolizin-7a(5H)-yl)methyl)- 5a,6,7,8,9,10-hexahydro-5H-4- oxa-1,10a,11,13,14-pentaaza- 6,9-methanonaphtho[1,8- ab]heptalen-12(13H)-one | 633 [M + H]⁺ |
| 112 | 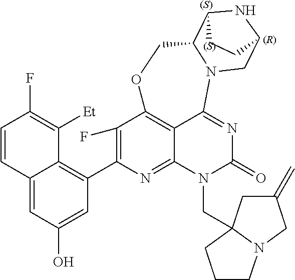 | (5aS,6S,9R)-13-((2,6- dimethylenetetrahydro-1H- pyrrolizin-7a(5H)-yl)methyl)-2- (8-ethyl-7-fluoro-3- hydroxynaphthalen-1-yl)-3- fluoro-5a,6,7,8,9,10-hexahydro- 5H-4-oxa-1,10a,11,13,14- pentaaza-6,9- methanonaphtho[1,8- ab]heptalen-12(13H)-one | 627 [M + H]⁺ |
| 113 | 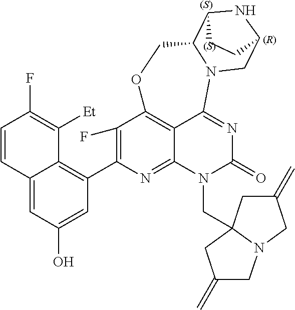 | (5aS,6S,9R)-2-(8-ethyl-7-fluoro- 3-hydroxynaphthalen-1-yl)-3- fluoro-13-(((2R,7aS)-2- fluorotetrahydro-1H-pyrrolizin- 7a(5H)-yl)methyl)-5a,6,7,8,9,10- hexahydro-5H-4-oxa- 1,10a,11,13,14-pentaaza-6,9- methanonaphtho[1,8- ab]heptalen-12(13H)-one | 639 [M + H]⁺ |
| 114 | 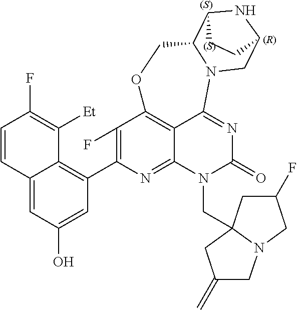 | (5aS,6S,9R)-2-(8-ethyl-7-fluoro- 3-hydroxynaphthalen-1-yl)-3- fluoro-13-((2- (fluoromethylene)tetrahydro- 1H-pyrrolizin-7a(5H)- yl)methyl)-5a,6,7,8,9,10- hexahydro-5H-4-oxa- 1,10a,11,13,14-pentaaza-6,9- methanonaphtho[1,8- ab]heptalen-12(13H)-one | 645 [M + H]⁺ |
| 115 | 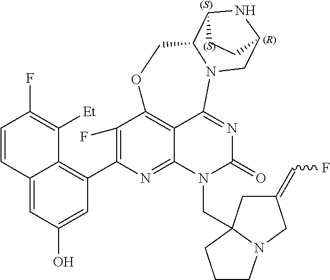 | (5aS,6S,9R)-2-(8-ethyl-7-fluoro- 3-hydroxynaphthalen-1-yl)-3- fluoro-13-((2-fluoro-6- methylenetetrahydro-1H- pyrrolizin-7a(5H)-yl)methyl)- 5a,6,7,8,9,10-hexahydro-5H-4- oxa-1,10a,11,13,14-pentaaza- 6,9-methanonaphtho[1,8- ab]heptalen-12(13H)-one | 645 [M + H]⁺ |
| 116 | 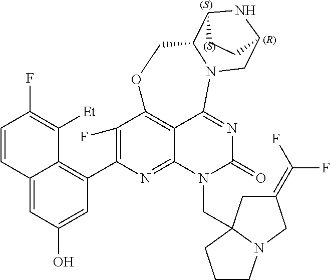 | (5aS,6S,9R)-13-((2- (difluoromethylene)tetrahydro- 1H-pyrrolizin-7a(5H)- yl)methyl)-2-(8-ethyl-7-fluoro- 3-hydroxynaphthalen-1-yl)-3- fluoro-5a,6,7,8,9,10-hexahydro- 5H-4-oxa-1,10a,11,13,14- pentaaza-6,9- methanonaphtho[1,8- ab]heptalen-12(13H)-one | 663 [M + H]⁺ |
| 117 | 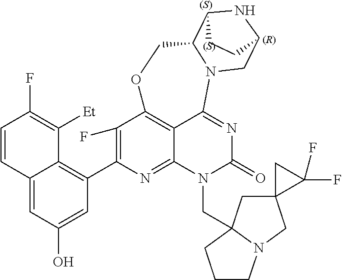 | (5aS,6S,9R)-13-((2,2- difluorodihydro-1′H,3′H- spiro[cyclopropane-1,2′- pyrrolizin]-7a′(5′H)-yl)methyl)- 2-(8-ethyl-7-fluoro-3- hydroxynaphthalen-1-yl)-3- fluoro-5a,6,7,8,9,10-hexahydro- 5H-4-oxa-1,10a,11,13,14- pentaaza-6,9- methanonaphtho[1,8- ab]heptalen-12(13H)-one | 677 [M + H]⁺ |
| 118 | 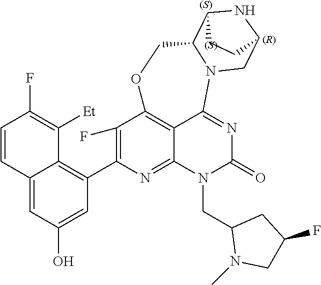 | (5aS,6S,9R)-2-(8-ethyl-7-fluoro- 3-hydroxynaphthalen-1-yl)-3- fluoro-13-(((4R)-4-fluoro-1- methylpyrrolidin-2-yl)methyl)- 5a,6,7,8,9,10-hexahydro-5H-4- oxa-1,10a,11,13,14-pentaaza- 6,9-methanonaphtho[1,8- ab]heptalen-12(13H)-one | 607 [M + H]⁺ |
| 119 | 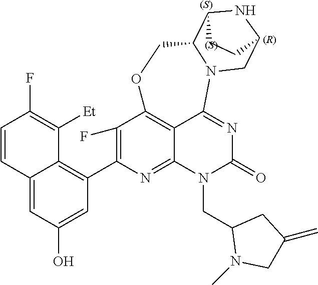 | (5aS,6S,9R)-2-(8-ethyl-7-fluoro- 3-hydroxynaphthalen-1-yl)-3- fluoro-13-((1-methyl-4- methylenepyrrolidin-2- yl)methyl)-5a,6,7,8,9,10- hexahydro-5H-4-oxa- 1,10a,11,13,14-pentaaza-6,9- methanonaphtho[1,8- ab]heptalen-12(13H)-one | 601 [M + H]⁺ |
| 120 | 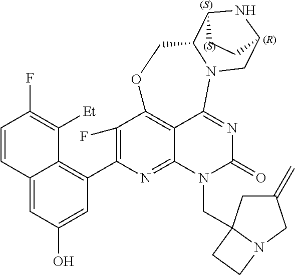 | (5aS,6S,9R)-2-(8-ethyl-7-fluoro- 3-hydroxynaphthalen-1-yl)-3- fluoro-13-((3-methylene-1- azabicyclo[3.2.0]heptan-5- yl)methyl)-5a,6,7,8,9,10- hexahydro-5H-4-oxa- 1,10a,11,13,14-pentaaza-6,9- methanonaphtho[1,8- ab]heptalen-12(13H)-one | 613 [M + H]⁺ |
| 121 | 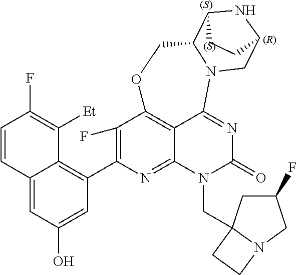 | (5aS,6S,9R)-2-(8-ethyl-7-fluoro- 3-hydroxynaphthalen-1-yl)-3- fluoro-13-(((3R)-3-fluoro-1- azabicyclo[3.2.0]heptan-5- yl)methyl)-5a,6,7,8,9,10- hexahydro-5H-4-oxa- 1,10a,11,13,14-pentaaza-6,9- methanonaphtho[1,8- ab]heptalen-12(13H)-one | 619 [M + H]⁺ |
| 122 | 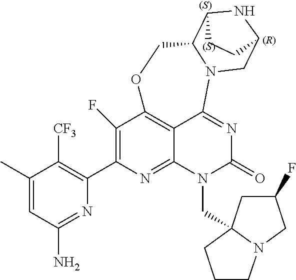 | (5aS,6S,9R)-2-(6-amino-4- methyl-3- (trifluoromethyl)pyridin-2-yl)-3- fluoro-13-((2- methylenetetrahydro-1H- pyrrolizin-7a(5H)-yl)methyl)- 5a,6,7,8,9,10-hexahydro-5H-4- oxa-1,10a,11,13,14-pentaaza- 6,9-methanonaphtho[1,8- ab]heptalen-12(13H)-one | 619 [M + H]⁺ |
| 123 | 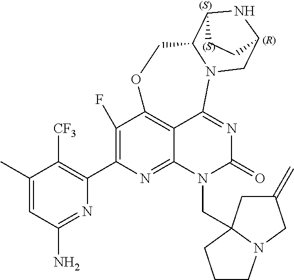 | (5aS,6S,9R)-2-(6-amino-4- methyl-3- (trifluoromethyl)pyridin-2-yl)- 13-((2,6-dimethylenetetrahydro- 1H-pyrrolizin-7a(5H)- yl)methyl)-3-fluoro- 5a,6,7,8,9,10-hexahydro-5H-4- oxa-1,10a,11,13,14-pentaaza- 6,9-methanonaphtho[1,8- ab]heptalen-12(13H)-one | 613 [M + H]⁺ |
| 124 | 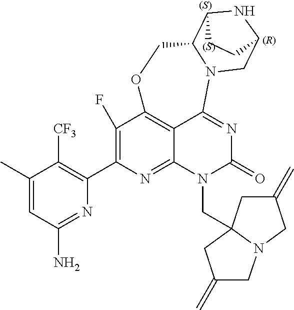 | (5aS,6S,9R)-2-(6-amino-4- methyl-3- (trifluoromethyl)pyridin-2-yl)-3- fluoro-13-(((2R,7aS)-2- fluorotetrahydro-1H-pyrrolizin- 7a(5H)-yl)methyl)-5a,6,7,8,9,10- hexahydro-5H-4-oxa- 1,10a,11,13,14-pentaaza-6,9- methanonaphtho[1,8- ab]heptalen-12(13H)-one | 625 [M + H]⁺ |
| 125 | 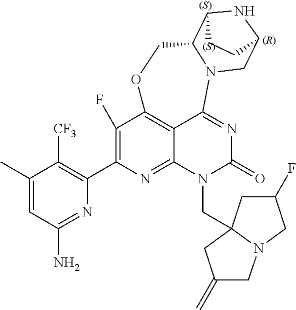 | (5aS,6S,9R)-2-(6-amino-4- methyl-3- (trifluoromethyl)pyridin-2-yl)-3- fluoro-13-((2- (fluoromethylene)tetrahydro- 1H-pyrrolizin-7a(5H)- yl)methyl)-5a,6,7,8,9,10- hexahydro-5H-4-oxa- 1,10a,11,13,14-pentaaza-6,9- methanonaphtho[1,8- ab]heptalen-12(13H)-one | 631 [M + H]⁺ |
| 126 | 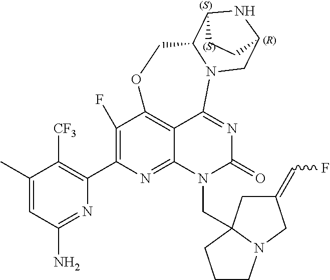 | (5aS,6S,9R)-2-(6-amino-4- methyl-3- (trifluoromethyl)pyridin-2-yl)-3- fluoro-13-((2-fluoro-6- methylenetetrahydro-1H- pyrrolizin-7a(5H)-yl)methyl)- 5a,6,7,8,9,10-hexahydro-5H-4- oxa-1,10a,11,13,14-pentaaza- 6,9-methanonaphtho[1,8- ab]heptalen-12(13H)-one | 631 [M + H]⁺ |
| 127 | 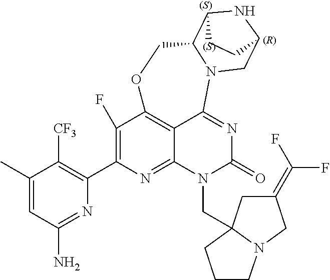 | (5aS,6S,9R)-2-(6-amino-4- methyl-3- (trifluoromethyl)pyridin-2-yl)- 13-((2- (difluoromethylene)tetrahydro- 1H-pyrrolizin-7a(5H)- yl)methyl)-3-fluoro- 5a,6,7,8,9,10-hexahydro-5H-4- oxa-1,10a,11,13,14-pentaaza- 6,9-methanonaphtho[1,8- ab]heptalen-12(13H)-one | 649 [M + H]⁺ |
| 128 | 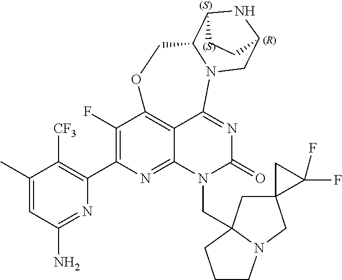 | (5aS,6S,9R)-2-(6-amino-4- methyl-3- (trifluoromethyl)pyridin-2-yl)- 13-((2,2-difluorodihydro- 1′H,3′H-spiro[cyclopropane-1,2′- pyrrolizin]-7a′(5′H)-yl)methyl)- 3-fluoro-5a,6,7,8,9,10- hexahydro-5H-4-oxa- 1,10a,11,13,14-pentaaza-6,9- methanonaphtho[1,8- ab]heptalen-12(13H)-one | 663 [M + H]⁺ |
| 129 | 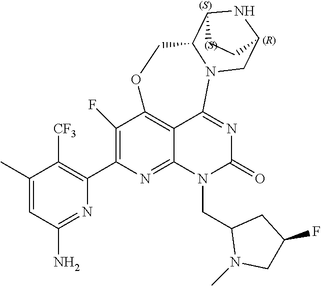 | (5aS,6S,9R)-2-(6-amino-4- methyl-3- (trifluoromethyl)pyridin-2-yl)-3- fluoro-13-(((4R)-4-fluoro-1- methylpyrrolidin-2-yl)methyl)- 5a,6,7,8,9,10-hexahydro-5H-4- oxa-1,10a,11,13,14-pentaaza- 6,9-methanonaphtho[1,8- ab]heptalen-12(13H)-one | 593 [M + H]⁺ |
| 130 | 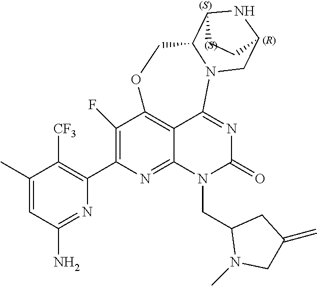 | (5aS,6S,9R)-2-(6-amino-4- methyl-3- (trifluoromethyl)pyridin-2-yl)-3- fluoro-13-((1-methyl-4- methylenepyrrolidin-2- yl)methyl)-5a,6,7,8,9,10- hexahydro-5H-4-oxa- 1,10a,11,13,14-pentaaza-6,9- methanonaphtho[1,8- ab]heptalen-12(13H)-one | 587 [M + H]⁺ |
| 131 | 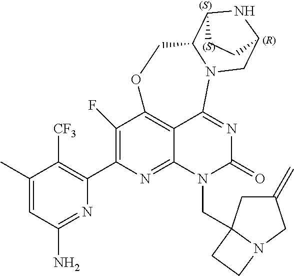 | (5aS,6S,9R)-2-(6-amino-4- methyl-3- (trifluoromethyl)pyridin-2-yl)-3- fluoro-13-((3-methylene-1- azabicyclo[3.2.0]heptan-5- yl)methyl)-5a,6,7,8,9,10- hexahydro-5H-4-oxa- 1,10a,11,13,14-pentaaza-6,9- methanonaphtho[1,8- ab]heptalen-12(13H)-one | 599 [M + H]⁺ |
| 132 | 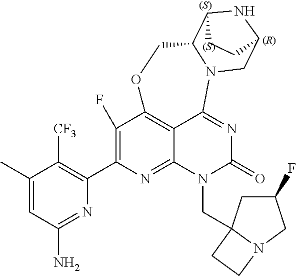 | (5aS,6S,9R)-2-(6-amino-4- methyl-3- (trifluoromethyl)pyridin-2-yl)-3- fluoro-13-(((3R)-3-fluoro-1- azabicyclo[3.2.0]heptan-5- yl)methyl)-5a,6,7,8,9,10- hexahydro-5H-4-oxa- 1,10a,11,13,14-pentaaza-6,9- methanonaphtho[1,8- ab]heptalen-12(13H)-one | 605 [M + H]⁺ |
| 133 | 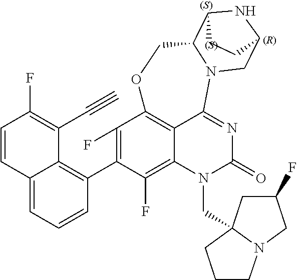 | (5aS,6S,9R)-2-(8-ethynyl-7- fluoronaphthalen-1-yl)-1,3- difluoro-14-((2- methylenetetrahydro-1H- pyrrolizin-7a(5H)-yl)methyl)- 5a,6,7,8,9,10-hexahydro-5H-6,9- epiminoazepino[2′,1′:3,4][1,4] oxazepino[5,6,7-de]quinazolin- 13(14H)-one | 630 [M + H]⁺ |
| 134 | 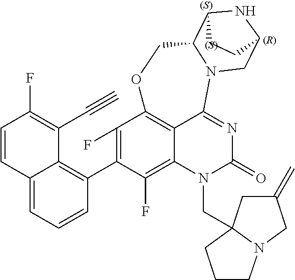 | (5aS,6S,9R)-14-((2,6- dimethylenetetrahydro-1H- pyrrolizin-7a(5H)-yl)methyl)-2- (8-ethynyl-7-fluoronaphthalen- 1-yl)-1,3-difluoro-5a,6,7,8,9,10- hexahydro-5H-6,9- epiminoazepino[2′,1′:3,4][1,4] oxazepino[5,6,7-de]quinazolin- 13(14H)-one | 624 [M + H]⁺ |
| 135 | 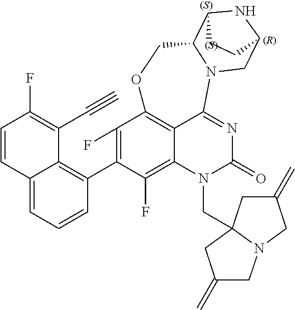 | (5aS,6S,9R)-2-(8-ethynyl-7- fluoronaphthalen-1-yl)-1,3- difluoro-14-(((2R,7aS)-2- fluorotetrahydro-1H-pyrrolizin- 7a(5H)-yl)methyl)-5a,6,7,8,9,10- hexahydro-5H-6,9- epiminoazepino[2′,1′:3,4][1,4] oxazepino[5,6,7-de]quinazolin- 13(14H)-one | 636 [M + H]⁺ |
| 136 | 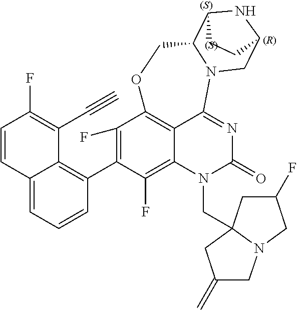 | (5aS,6S,9R)-2-(8-ethynyl-7- fluoronaphthalen-1-yl)-1,3- difluoro-14-((2- (fluoromethylene)tetrahydro- 1H-pyrrolizin-7a(5H)- yl)methyl)-5a,6,7,8,9,10- hexahydro-5H-6,9- epiminoazepino[2′,1′:3,4][1,4] oxazepino[5,6,7-de]quinazolin- 13(14H)-one | 642 [M + H]⁺ |
| 137 | 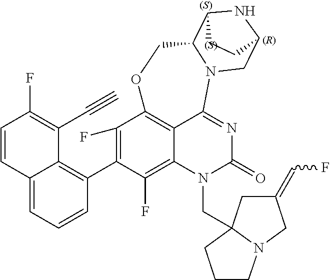 | (5aS,6S,9R)-2-(8-ethynyl-7- fluoronaphthalen-1-yl)-1,3- difluoro-14-((2-fluoro-6- methylenetetrahydro-1H- pyrrolizin-7a(5H)-yl)methyl)- 5a,6,7,8,9,10-hexahydro-5H-6,9- epiminoazepino[2′,1′:3,4][1,4] oxazepino[5,6,7-de]quinazolin- 13(14H)-one | 642 [M + H]⁺ |
| 138 | 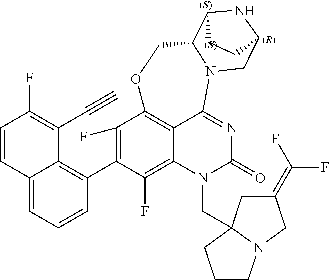 | (5aS,6S,9R)-14-((2- (difluoromethylene)tetrahydro- 1H-pyrrolizin-7a(5H)- yl)methyl)-2-(8-ethynyl-7- fluoronaphthalen-1-yl)-1,3- difluoro-5a,6,7,8,9,10- hexahydro-5H-6,9- epiminoazepino[2′,1′:3,4][1,4] oxazepino[5,6,7-de]quinazolin- 13(14H)-one | 660 [M + H]⁺ |
| 139 | 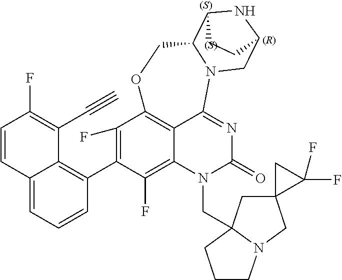 | (5aS,6S,9R)-14-((2,2- difluorodihydro-1′H,3′H- spiro[cyclopropane-1,2′- pyrrolizin]-7a′(5′H)-yl)methyl)- 2-(8-ethynyl-7- fluoronaphthalen-1-yl)-1,3- difluoro-5a,6,7,8,9,10- hexahydro-5H-6,9- epiminoazepino[2′,1′:3,4][1,4] oxazepino[5,6,7-de]quinazolin- 13(14H)-one | 674 [M + H]⁺ |
| 140 | 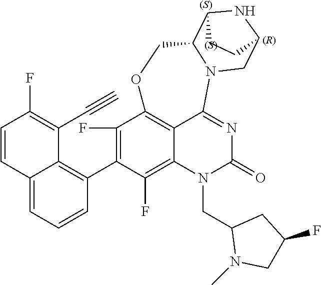 | (5aS,6S,9R)-2-(8-ethynyl-7- fluoronaphthalen-1-yl)-1,3- difluoro-14-(((4R)-4-fluoro-1- methylpyrrolidin-2-yl)methyl)- 5a,6,7,8,9,10-hexahydro-5H-6,9- epiminoazepino[2′,1′:3,4][1,4] oxazepino[5,6,7-de]quinazolin- 13(14H)-one | 604 [M + H]⁺ |
| 141 | 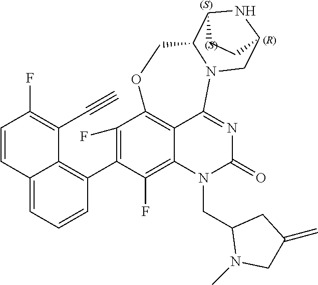 | (5aS,6S,9R)-2-(8-ethynyl-7- fluoronaphthalen-1-yl)-1,3- difluoro-14-((1-methyl-4- methylenepyrrolidin-2- yl)methyl)-5a,6,7,8,9,10- hexahydro-5H-6,9- epiminoazepino[2′,1′:3,4][1,4] oxazepino[5,6,7-de]quinazolin- 13(14H)-one | 598 [M + H]⁺ |
| 142 | 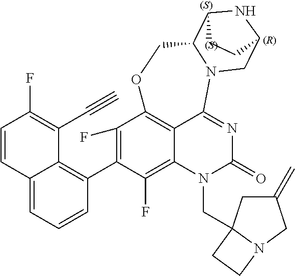 | (5aS,6S,9R)-2-(8-ethynyl-7- fluoronaphthalen-1-yl)-1,3- difluoro-14-((3-methylene-1- azabicyclo[3.2.0]heptan-5- yl)methyl)-5a,6,7,8,9,10- hexahydro-5H-6,9- epiminoazepino[2′,1′:3,4][1,4] oxazepino[5,6,7-de]quinazolin- 13(14H)-one | 610 [M + H]⁺ |
| 143 | 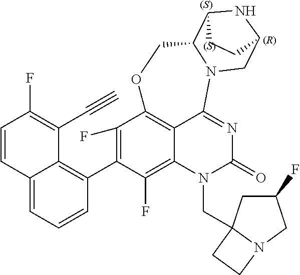 | (5aS,6S,9R)-2-(8-ethynyl-7- fluoronaphthalen-1-yl)-1,3- difluoro-14-(((3R)-3-fluoro-1- azabicyclo[3.2.0]heptan-5- yl)methyl)-5a,6,7,8,9,10- hexahydro-5H-6,9- epiminoazepino[2′,1′:3,4][1,4] oxazepino[5,6,7-de]quinazolin- 13(14H)-one | 616 [M + H]⁺ |
| 144 | 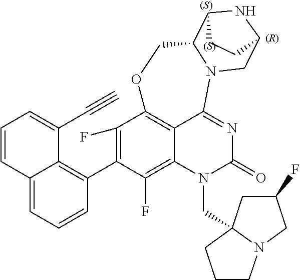 | (5aS,6S,9R)-2-(8- ethynylnaphthalen-1-yl)-1,3- difluoro-14-((2- methylenetetrahydro-1H- pyrrolizin-7a(5H)-yl)methyl)- 5a,6,7,8,9,10-hexahydro-5H-6,9- epiminoazepino[2′,1′:3,4][1,4] oxazepino[5,6,7-de]quinazolin- 13(14H)-one | 612 [M + H]⁺ |
| 145 | 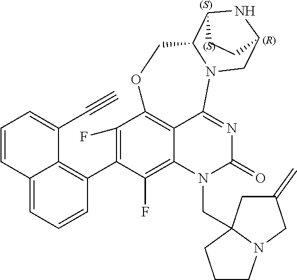 | (5aS,6S,9R)-14-((2,6- dimethylenetetrahydro-1H- pyrrolizin-7a(5H)-yl)methyl)-2- (8-ethynylnaphthalen-1-yl)-1,3- difluoro-5a,6,7,8,9,10- hexahydro-5H-6,9- epiminoazepino[2′,1′:3,4][1,4] oxazepino[5,6,7-de]quinazolin- 13(14H)-one | 606 [M + H]⁺ |
| 146 | 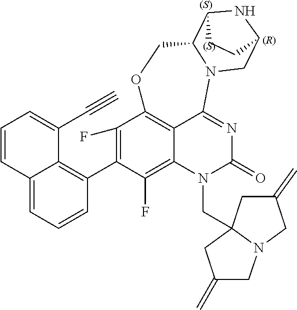 | (5aS,6S,9R)-2-(8- ethynylnaphthalen-1-yl)-1,3- difluoro-14-(((2R,7aS)-2- fluorotetrahydro-1H-pyrrolizin- 7a(5H)-yl)methyl)-5a,6,7,8,9,10- hexahydro-5H-6,9- epiminoazepino[2′,1′:3,4][1,4] oxazepino[5,6,7-de]quinazolin- 13(14H)-one | 618 [M + H]⁺ |
| 147 | 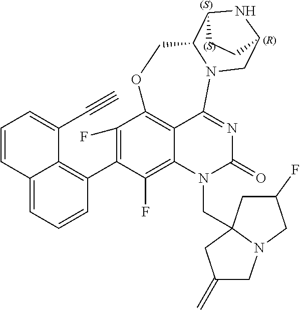 | (5aS,6S,9R)-2-(8- ethynylnaphthalen-1-yl)-1,3- difluoro-14-((2- (fluoromethylene)tetrahydro- 1 H-pyrrolizin-7a(5H)- yl)methyl)-5a,6,7,8,9,10- hexahydro-5H-6,9- epiminoazepino[2′,1′:3,4][1,4] oxazepino[5,6,7-de]quinazolin- 13(14H)-one | 624 [M + H]⁺ |
| 148 | 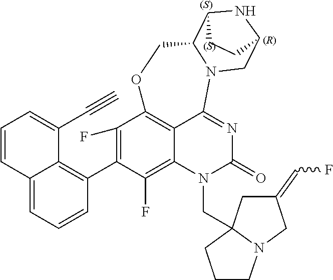 | (5aS,6S,9R)-2-(8- ethynylnaphthalen-1-yl)-1,3- difluoro-14-((2-fluoro-6- methylenetetrahydro-1H- pyrrolizin-7a(5H)-yl)methyl)- 5a,6,7,8,9,10-hexahydro-5H-6,9- epiminoazepino[2′,1′:3,4][1,4] oxazepino[5,6,7-de]quinazolin- 13(14H)-one | 624 [M + H]⁺ |
| 149 | 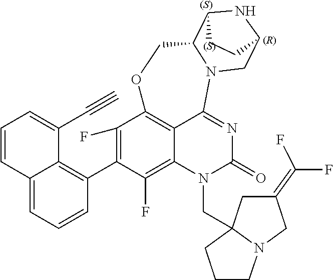 | (5aS,6S,9R)-14-((2- (difluoromethylene)tetrahydro- 1H-pyrrolizin-7a(5H)- yl)methyl)-2-(8- ethynylnaphthalen-1-yl)-1,3- difluoro-5a,6,7,8,9,10- hexahydro-5H-6,9- epiminoazepino[2′,1′:3,4][1,4] oxazepino[5,6,7-de]quinazolin- 13(14H)-one | 642 [M + H]⁺ |
| 150 | 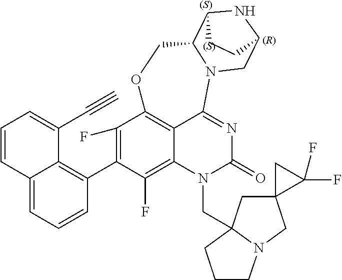 | (5aS,6S,9R)-14-((2,2- difluorodihydro-1′H,3′H- spiro[cyclopropane-1,2′- pyrrolizin]-7a′(5′H)-yl)methyl)- 2-(8-ethynylnaphthalen-1-yl)- 1,3-difluoro-5a,6,7,8,9,10- hexahydro-5H-6,9- epiminoazepino[2′,1′:3,4][1,4] oxazepino[5,6,7-de]quinazolin- 13(14H)-one | 656 [M + H]⁺ |
| 151 | 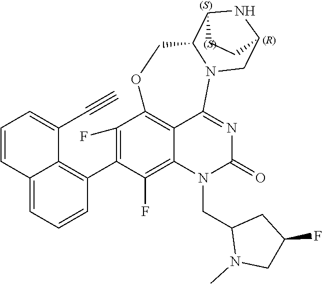 | (5aS,6S,9R)-2-(8- ethynylnaphthalen-1-yl)-1,3- difluoro-14-(((4R)-4-fluoro-1- methylpyrrolidin-2-yl)methyl)- 5a,6,7,8,9,10-hexahydro-5H-6,9- epiminoazepino[2′,1′:3,4][1,4] oxazepino[5,6,7-de]quinazolin- 13(14H)-one | 586 [M + H]⁺ |
| 152 | 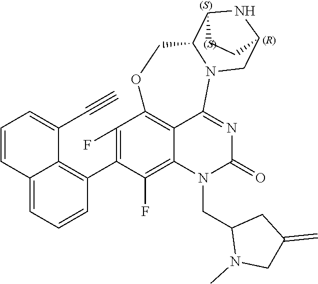 | (5aS,6S,9R)-2-(8- ethynylnaphthalen-1-yl)-1,3- difluoro-14-((1-methyl-4- methylenepyrrolidin-2- yl)methyl)-5a,6,7,8,9,10- hexahydro-5H-6,9- epiminoazepino[2′,1′:3,4][1,4] oxazepino[5,6,7-de]quinazolin- 13(14H)-one | 580 [M + H]⁺ |
| 153 | 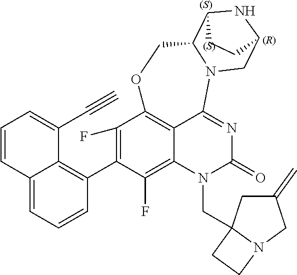 | (5aS,6S,9R)-2-(8- ethynylnaphthalen-1-yl)-1,3- difluoro-14-((3-methylene-1- azabicyclo[3.2.0]heptan-5- yl)methyl)-5a,6,7,8,9,10- hexahydro-5H-6,9- epiminoazepino[2′,1′:3,4][1,4] oxazepino[5,6,7-de]quinazolin- 13(14H)-one | 592 [M + H]⁺ |
| 154 | 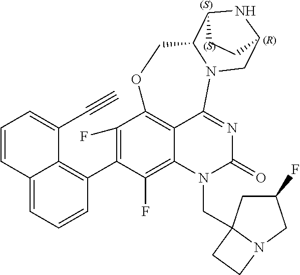 | (5aS,6S,9R)-2-(8- ethynylnaphthalen-1-yl)-1,3- difluoro-14-(((3R)-3-fluoro-1- azabicyclo[3.2.0]heptan-5- yl)methyl)-5a,6,7,8,9,10- hexahydro-5H-6,9- epiminoazepino[2′,1′:3,4][1,4] oxazepino[5,6,7-de]quinazolin- 13(14H)-one | 598 [M + H]⁺ |
| 155 | 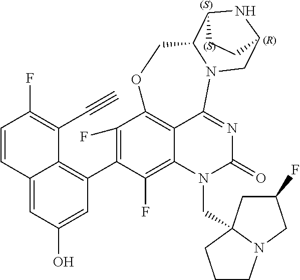 | (5aS,6S,9R)-2-(8-ethynyl-7- fluoro-3-hydroxynaphthalen-1- yl)-1,3-difluoro-14-((2- methylenetetrahydro-1H- pyrrolizin-7a(5H)-yl)methyl)- 5a,6,7,8,9,10-hexahydro-5H-6,9- epiminoazepino[2′,1′:3,4][1,4] oxazepino[5,6,7-de]quinazolin- 13(14H)-one | 646 [M + H]⁺ |
| 156 | 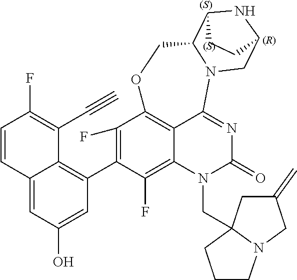 | (5aS,6S,9R)-14-((2,6- dimethylenetetrahydro-1H- pyrrolizin-7a(5H)-yl)methyl)-2- (8-ethynyl-7-fluoro-3- hydroxynaphthalen-1-yl)-1,3- difluoro-5a,6,7,8,9,10- hexahydro-5H-6,9- epiminoazepino[2′,1′:3,4][1,4] oxazepino[5,6,7-de]quinazolin- 13(14H)-one | 640 [M + H]⁺ |
| 157 | 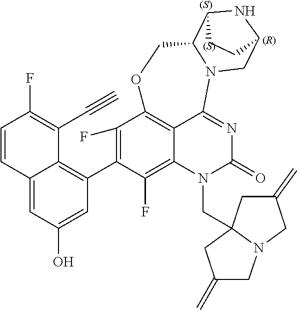 | (5aS,6S,9R)-2-(8-ethynyl-7- fluoro-3-hydroxynaphthalen-1- yl)-1,3-difluoro-14-(((2R,7aS)- 2-fluorotetrahydro-1H- pyrrolizin-7a(5H)-yl)methyl)- 5a,6,7,8,9,10-hexahydro-5H-6,9- epiminoazepino[2′,1′:3,4][1,4] oxazepino[5,6,7-de]quinazolin- 13(14H)-one | 652 [M + H]⁺ |
| 158 | 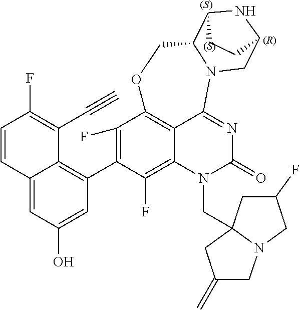 | (5aS,6S,9R)-2-(8-ethynyl-7- fluoro-3-hydroxynaphthalen-1- yl)-1,3-difluoro-14-((2- (fluoromethylene)tetrahydro- 1H-pyrrolizin-7a(5H)- yl)methyl)-5a,6,7,8,9,10- hexahydro-5H-6,9- epiminoazepino[2′,1′:3,4][1,4] oxazepino[5,6,7-de]quinazolin- 13(14H)-one | 658 [M + H]⁺ |
| 159 | 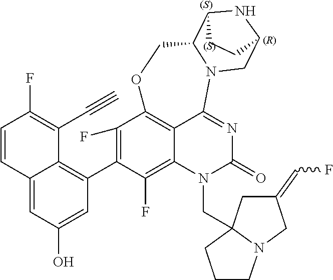 | (5aS,6S,9R)-2-(8-ethynyl-7- fluoro-3-hydroxynaphthalen-1- yl)-1,3-difluoro-14-((2-fluoro-6- methylenetetrahydro-1H- pyrrolizin-7a(5H)-yl)methyl)- 5a,6,7,8,9,10-hexahydro-5H-6,9- epiminoazepino[2′,1′:3,4][1,4] oxazepino[5,6,7-de]quinazolin- 13(14H)-one | 658 [M + H]⁺ |
| 160 | 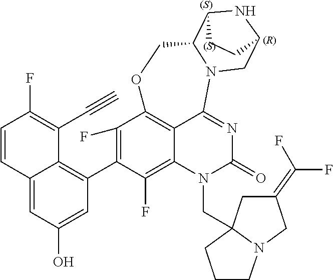 | (5aS,6S,9R)-14-((2- (difluoromethylene)tetrahydro- 1H-pyrrolizin-7a(5H)- yl)methyl)-2-(8-ethynyl-7- fluoro-3-hydroxynaphthalen-1- yl)-1,3-difluoro-5a,6,7,8,9,10- hexahydro-5H-6,9- epiminoazepino[2′,1′:3,4][1,4] oxazepino[5,6,7-de]quinazolin- 13(14H)-one | 676 [M + H]⁺ |
| 161 | 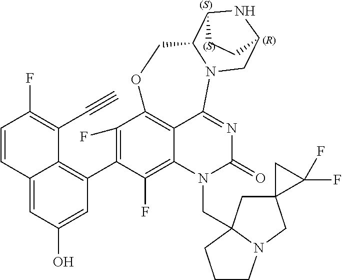 | (5aS,6S,9R)-14-((2,2- difluorodihydro-1′H,3′H- spiro[cyclopropane-1,2′- pyrrolizin]-7a′(5′H)-yl)methyl)- 2-(8-ethynyl-7-fluoro-3- hydroxynaphthalen-1-yl)-1,3- difluoro-5a,6,7,8,9,10- hexahydro-5H-6,9- epiminoazepino[2′,1′:3,4][1,4] oxazepino[5,6,7-de]quinazolin- 13(14H)-one | 690 [M + H]⁺ |
| 162 | 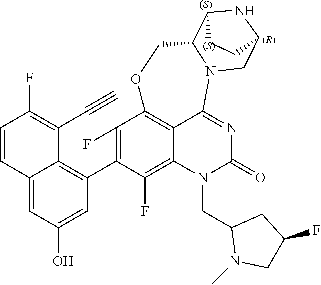 | (5aS,6S,9R)-2-(8-ethynyl-7- fluoro-3-hydroxynaphthalen-1- yl)-1,3-difluoro-14-(((4R)-4- fluoro-1-methylpyrrolidin-2- yl)methyl)-5a,6,7,8,9,10- hexahydro-5H-6,9- epiminoazepino[2′,1′:3,4][1,4] oxazepino[5,6,7-de]quinazolin- 13(14H)-one | 620 [M + H]⁺ |
| 163 | 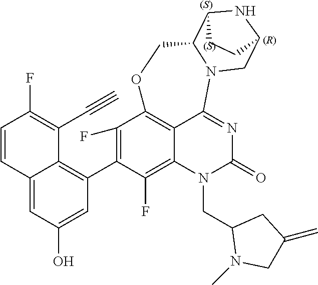 | (5aS,6S,9R)-2-(8-ethynyl-7- fluoro-3-hydroxynaphthalen-1- yl)-1,3-difluoro-14-((1-methyl- 4-methylenepyrrolidin-2- yl)methyl)-5a,6,7,8,9,10- hexahydro-5H-6,9- epiminoazepino[2′,1′:3,4][1,4] oxazepino[5,6,7-de]quinazolin- 13(14H)-one | 614 [M + H]⁺ |
| 164 | 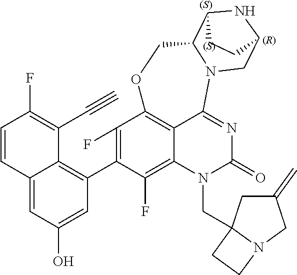 | (5aS,6S,9R)-2-(8-ethynyl-7- fluoro-3-hydroxynaphthalen-1- yl)-1,3-difluoro-14-((3- methylene-1- azabicyclo[3.2.0]heptan-5- yl)methyl)-5a,6,7,8,9,10- hexahydro-5H-6,9- epiminoazepino[2′,1′:3,4][1,4] oxazepino[5,6,7-de]quinazolin- 13(14H)-one | 626 [M + H]⁺ |
| 165 | 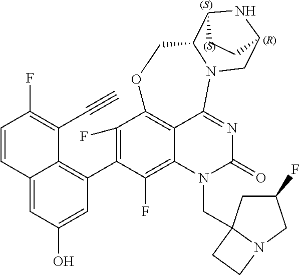 | (5aS,6S,9R)-2-(8-ethynyl-7- fluoro-3-hydroxynaphthalen-1- yl)-1,3-difluoro-14-(((3R)-3- fluoro-1- azabicyclo[3.2.0]heptan-5- yl)methyl)-5a,6,7,8,9,10- hexahydro-5H-6,9- epiminoazepino[2′,1′:3,4][1,4] oxazepino[5,6,7-de]quinazolin- 13(14H)-one | 632 [M + H]⁺ |
| 166 | 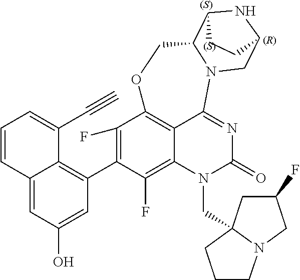 | (5aS,6S,9R)-2-(8-ethynyl-3- hydroxynaphthalen-1-yl)-1,3- difluoro-14-((2- methylenetetrahydro-1H- pyrrolizin-7a(5H)-yl)methyl)- 5a,6,7,8,9,10-hexahydro-5H-6,9- epiminoazepino[2′,1′:3,4][1,4] oxazepino[5,6,7-de]quinazolin- 13(14H)-one | 628 [M + H]⁺ |
| 167 | 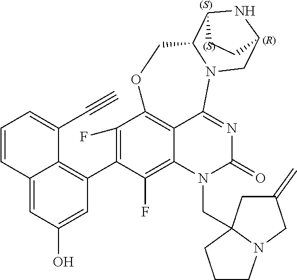 | (5aS,6S,9R)-14-((2,6- dimethylenetetrahydro-1H- pyrrolizin-7a(5H)-yl)methyl)-2- (8-ethynyl-3- hydroxynaphthalen-1-yl)-1,3- difluoro-5a,6,7,8,9,10- hexahydro-5H-6,9- epiminoazepino[2′,1′:3,4][1,4] oxazepino[5,6,7-de]quinazolin- 13(14H)-one | 622 [M + H]⁺ |
| 168 | 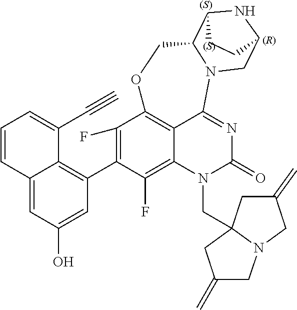 | (5aS,6S,9R)-2-(8-ethynyl-3- hydroxynaphthalen-1-yl)-1,3- difluoro-14-(((2R,7aS)-2- fluorotetrahydro-1H-pyrrolizin- 7a(5H)-yl)methyl)-5a,6,7,8,9,10- hexahydro-5H-6,9- epiminoazepino[2′,1′:3,4][1,4] oxazepino[5,6,7-de]quinazolin- 13(14H)-one | 634 [M + H]⁺ |
| 169 | 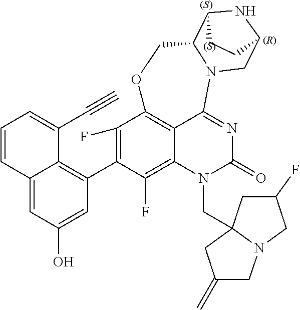 | (5aS,6S,9R)-2-(8-ethynyl-3- hydroxynaphthalen-1-yl)-1,3- difluoro-14-((2- (fluoromethylene)tetrahydro- 1H-pyrrolizin-7a(5H)- yl)methyl)-5a,6,7,8,9,10- hexahydro-5H-6,9- epiminoazepino[2′,1′:3,4][1,4] oxazepino[5,6,7-de]quinazolin- 13(14H)-one | 640 [M + H]⁺ |
| 170 | 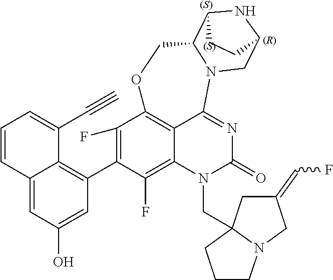 | (5aS,6S,9R)-2-(8-ethynyl-3- hydroxynaphthalen-1-yl)-1,3- difluoro-14-((2-fluoro-6- methylenetetrahydro-1H- pyrrolizin-7a(5H)-yl)methyl)- 5a,6,7,8,9,10-hexahydro-5H-6,9- epiminoazepino[2′,1′:3,4][1,4] oxazepino[5,6,7-de]quinazolin- 13(14H)-one | 640 [M + H]⁺ |
| 171 | 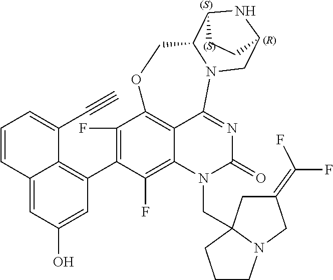 | (5aS,6S,9R)-14-((2- (difluoromethylene)tetrahydro- 1H-pyrrolizin-7a(5H)- yl)methyl)-2-(8-ethynyl-3- hydroxynaphthalen-1-yl)-1,3- difluoro-5a,6,7,8,9,10- hexahydro-5H-6,9- epiminoazepino[2′,1′:3,4][1,4] oxazepino[5,6,7-de]quinazolin- 13(14H)-one | 658 [M + H]⁺ |
| 172 | 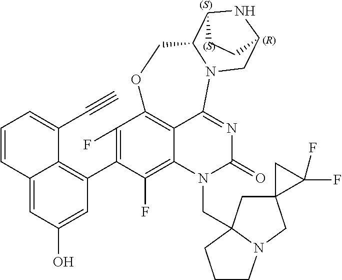 | (5aS,6S,9R)-14-((2,2- difluorodihydro-1′H,3′H- spiro[cyclopropane-1,2′- pyrrolizin]-7a′(5′H)-yl)methyl)- 2-(8-ethynyl-3- hydroxynaphthalen-1-yl)-1,3- difluoro-5a,6,7,8,9,10- hexahydro-5H-6,9- epiminoazepino[2′,1′:3,4][1,4] oxazepino[5,6,7-de]quinazolin- 13(14H)-one | 672 [M + H]⁺ |
| 173 | 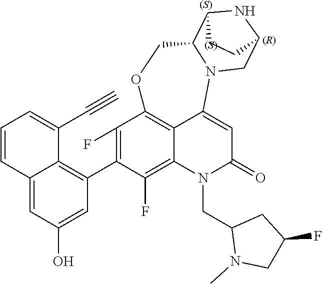 | (5aS,6S,9R)-2-(8-ethynyl-3- hydroxynaphthalen-1-yl)-1,3- difluoro-14-(((4R)-4-fluoro-1- methylpyrrolidin-2-yl)methyl)- 5a,6,7,8,9,10-hexahydro-5H-6,9- epiminoazepino[2′,1′:3,4][1,4] oxazepino[5,6,7-de]quinazolin- 13(14H)-one | 602 [M + H]⁺ |
| 174 | 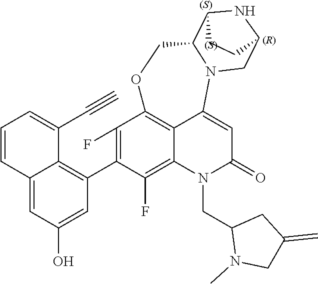 | (5aS,6S,9R)-2-(8-ethynyl-3- hydroxynaphthalen-1-yl)-1,3- difluoro-14-((1-methyl-4- methylenepyrrolidin-2- yl)methyl)-5a,6,7,8,9,10- hexahydro-5H-6,9- epiminoazepino[2′,1′:3,4][1,4] oxazepino[5,6,7-de]quinazolin- 13(14H)-one | 596 [M + H]⁺ |
| 175 | 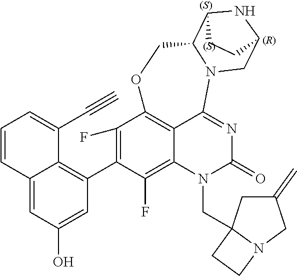 | (5aS,6S,9R)-2-(8-ethynyl-3- hydroxynaphthalen-1-yl)-1,3- difluoro-14-((3-methylene-1- azabicyclo[3.2.0]heptan-5- yl)methyl)-5a,6,7,8,9,10- hexahydro-5H-6,9- epiminoazepino[2′,1′:3,4][1,4] oxazepino[5,6,7-de]quinazolin- 13(14H)-one | 608 [M + H]⁺ |
| 176 | 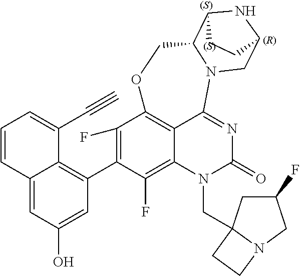 | (5aS,6S,9R)-2-(8-ethynyl-3- hydroxynaphthalen-1-yl)-1,3- difluoro-14-(((3R)-3-fluoro-1- azabicyclo[3.2.0]heptan-5- yl)methyl)-5a,6,7,8,9,10- hexahydro-5H-6,9- epiminoazepino[2′,1′:3,4][1,4] oxazepino[5,6,7-de]quinazolin- 13(14H)-one | 614 [M + H]⁺ |
| 177 | 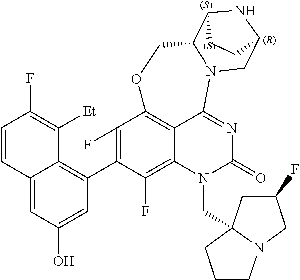 | (5aS,6S,9R)-2-(8-ethyl-7-fluoro- 3-hydroxynaphthalen-1-yl)-1,3- difluoro-14-((2- methylenetetrahydro-1H- pyrrolizin-7a(5H)-yl)methyl)- 5a,6,7,8,9,10-hexahydro-5H-6,9- epiminoazepino[2′,1′:3,4][1,4] oxazepino[5,6,7-de]quinazolin- 13(14H)-one | 650 [M + H]⁺ |
| 179 | 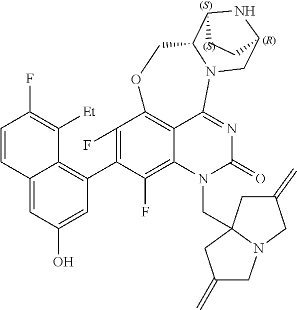 | (5aS,6S,9R)-2-(8-ethyl-7-fluoro- 3-hydroxynaphthalen-1-yl)-1,3- difluoro-14-(((2R,7aS)-2- fluorotetrahydro-1H-pyrrolizin- 7a(5H)-yl)methyl)-5a,6,7,8,9,10- hexahydro-5H-6,9- epiminoazepino[2′,1′:3,4][1,4] oxazepino[5,6,7-de]quinazolin- 13(14H)-one | 656 [M + H]⁺ |
| 180 | 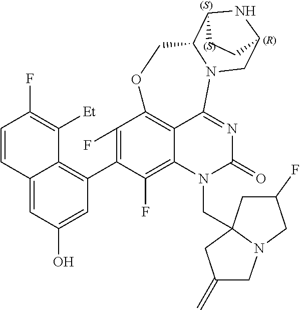 | (5aS,6S,9R)-2-(8-ethyl-7-fluoro- 3-hydroxynaphthalen-1-yl)-1,3- difluoro-14-((2- (fluoromethylene)tetrahydro- 1H-pyrrolizin-7a(5H)- yl)methyl)-5a,6,7,8,9,10- hexahydro-5H-6,9- epiminoazepino[2′,1′:3,4][1,4] oxazepino[5,6,7-de]quinazolin- 13(14H)-one | 662 [M + H]⁺ |
| 181 | 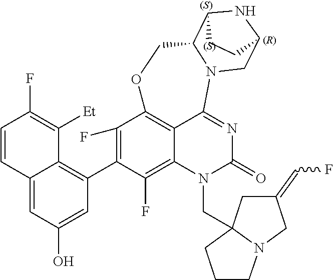 | (5aS,6S,9R)-2-(8-ethyl-7-fluoro- 3-hydroxynaphthalen-1-yl)-1,3- difluoro-14-((2-fluoro-6- methylenetetrahydro-1H- pyrrolizin-7a(5H)-yl)methyl)- 5a,6,7,8,9,10-hexahydro-5H-6,9- epiminoazepino[2′,1′:3,4][1,4] oxazepino[5,6,7-de]quinazolin- 13(14H)-one | 662 [M + H]⁺ |
| 182 | 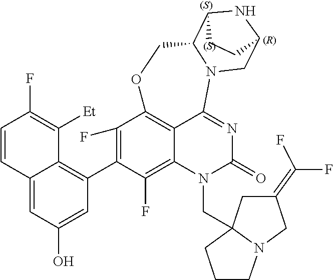 | (5aS,6S,9R)-14-((2- (difluoromethylene)tetrahydro- 1H-pyrrolizin-7a(5H)- yl)methyl)-2-(8-ethyl-7-fluoro- 3-hydroxynaphthalen-1-yl)-1,3- difluoro-5a,6,7,8,9,10- hexahydro-5H-6,9- epiminoazepino[2′,1′:3,4][1,4] oxazepino[5,6,7-de]quinazolin- 13(14H)-one | 680 [M + H]⁺ |
| 183 | 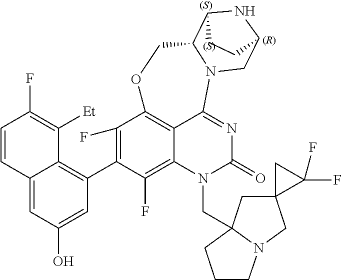 | (5aS,6S,9R)-14-((2,2- difluorodihydro-1′H,3′H- spiro[cyclopropane-1,2′- pyrrolizin]-7a′(5′H)-yl)methyl)- 2-(8-ethyl-7-fluoro-3- hydroxynaphthalen-1-yl)-1,3- difluoro-5a,6,7,8,9,10- hexahydro-5H-6,9- epiminoazepino[2′,1′:3,4][1,4] oxazepino[5,6,7-de]quinazolin- 13(14H)-one | 694 [M + H]⁺ |
| 184 | 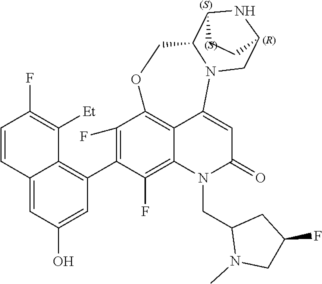 | (5aS,6S,9R)-2-(8-ethyl-7-fluoro- 3-hydroxynaphthalen-1-yl)-1,3- difluoro-14-(((4R)-4-fluoro-1- methylpyrrolidin-2-yl)methyl)- 5a,6,7,8,9,10-hexahydro-5H-6,9- epiminoazepino[2′,1′:3,4][1,4] oxazepino[5,6,7-de]quinazolin- 13(14H)-one | 624 [M + H]⁺ |
| 185 | 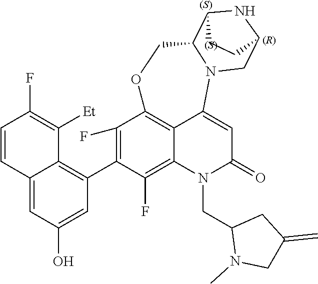 | (5aS,6S,9R)-2-(8-ethyl-7-fluoro- 3-hydroxynaphthalen-1-yl)-1,3- difluoro-14-((1-methyl-4- methylenepyrrolidin-2- yl)methyl)-5a,6,7,8,9,10- hexahydro-5H-6,9- epiminoazepino[2′,1′:3,4][1,4] oxazepino[5,6,7-de]quinazolin- 13(14H)-one | 618 [M + H]⁺ |
| 186 | 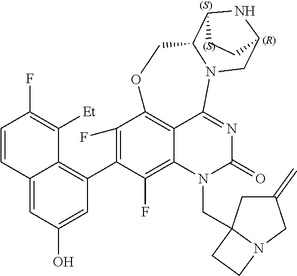 | (5aS,6S,9R)-2-(8-ethyl-7-fluoro- 3-hydroxynaphthalen-1-yl)-1,3- difluoro-14-((3-methylene-1- azabicyclo[3.2.0]heptan-5- yl)methyl)-5a,6,7,8,9,10- hexahydro-5H-6,9- epiminoazepino[2′,1′:3,4][1,4] oxazepino[5,6,7-de]quinazolin- 13(14H)-one | 630 [M + H]⁺ |
| 187 | 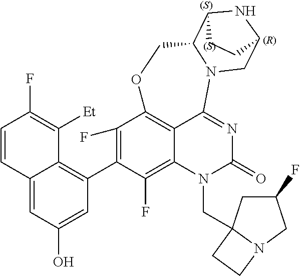 | (5aS,6S,9R)-2-(8-ethyl-7-fluoro- 3-hydroxynaphthalen-1-yl)-1,3- difluoro-14-(((3R)-3-fluoro-1- azabicyclo[3.2.0]heptan-5- yl)methyl)-5a,6,7,8,9,10- hexahydro-5H-6,9- epiminoazepino[2′,1′:3,4][1,4] oxazepino[5,6,7-de]quinazolin- 13(14H)-one | 636 [M + H]⁺ |
| 188 | 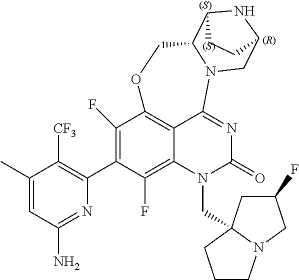 | (5aS,6S,9R)-2-(6-amino-4- methyl-3- (trifluoromethyl)pyridin-2-yl)- 1,3-difluoro-14-((2- methylenetetrahydro-1H- pyrrolizin-7a(5H)-yl)methyl)- 5a,6,7,8,9,10-hexahydro-5H-6,9- epiminoazepino[2′,1′:3,4][1,4] oxazepino[5,6,7-de]quinazolin- 13(14H)-one | 636 [M + H]⁺ |
| 189 | 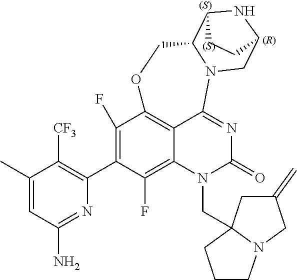 | (5aS,6S,9R)-2-(6-amino-4- methyl-3- (trifluoromethyl)pyridin-2-yl)- 14-((2,6-dimethylenetetrahydro- 1H-pyrrolizin-7a(5H)- yl)methyl)-1,3-difluoro- 5a,6,7,8,9,10-hexahydro-5H-6,9- epiminoazepino[2′,1′:3,4][1,4] oxazepino[5,6,7-de]quinazolin- 13(14H)-one | 630 [M + H]⁺ |
| 190 | 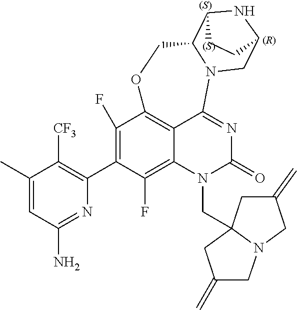 | (5aS,6S,9R)-2-(6-amino-4- methyl-3- (trifluoromethyl)pyridin-2-yl)- 1,3-difluoro-14-(((2R,7aS)-2- fluorotetrahydro-1H-pyrrolizin- 7a(5H)-yl)methyl)-5a,6,7,8,9,10- hexahydro-5H-6,9- epiminoazepino[2′,1′:3,4][1,4] oxazepino[5,6,7-de]quinazolin- 13(14H)-one | 642 [M + H]⁺ |
| 191 | 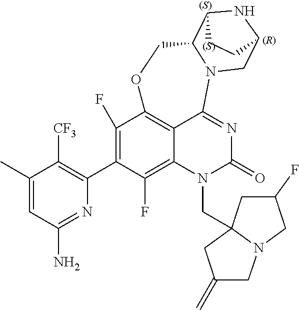 | (5aS,6S,9R)-2-(6-amino-4- methyl-3- (trifluoromethyl)pyridin-2-yl)- 1,3-difluoro-14-((2- (fluoromethylene)tetrahydro- 1H-pyrrolizin-7a(5H)- yl)methyl)-5a,6,7,8,9,10- hexahydro-5H-6,9- epiminoazepino[2′,1′:3,4][1,4] oxazepino[5,6,7-de]quinazolin- 13(14H)-one | 648 [M + H]⁺ |
| 192 | 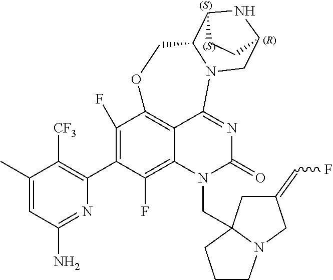 | (5aS,6S,9R)-2-(6-amino-4- methyl-3- (trifluoromethyl)pyridin-2-yl)- 1,3-difluoro-14-((2-fluoro-6- methylenetetrahydro-1H- pyrrolizin-7a(5H)-yl)methyl)- 5a,6,7,8,9,10-hexahydro-5H-6,9- epiminoazepino[2′,1′:3,4][1,4] oxazepino[5,6,7-de]quinazolin- 13(14H)-one | 648 [M + H]⁺ |
| 193 | 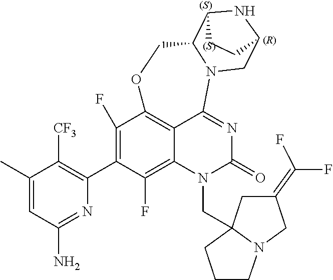 | (5aS,6S,9R)-2-(6-amino-4- methyl-3- (trifluoromethyl)pyridin-2-yl)- 14-((2- (difluoromethylene)tetrahydro- 1H-pyrrolizin-7a(5H)- yl)methyl)-1,3-difluoro- 5a,6,7,8,9,10-hexahydro-5H-6,9- epiminoazepino[2′,1′:3,4][1,4] oxazepino[5,6,7-de]quinazolin- 13(14H)-one | 666 [M + H]⁺ |
| 194 | 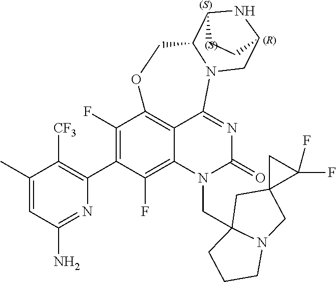 | (5aS,6S,9R)-2-(6-amino-4- methyl-3- (trifluoromethyl)pyridin-2-yl)- 14-((2,2-difluorodihydro- 1′H,3′H-spiro[cyclopropane-1,2′- pyrrolizin]-7a′(5′H)-yl)methyl)- 1,3-difluoro-5a,6,7,8,9,10- hexahydro-5H-6,9- epiminoazepino[2′,1′:3,4][1,4] oxazepino[5,6,7-de]quinazolin- 13(14H)-one | 680 [M + H]⁺ |
| 195 | 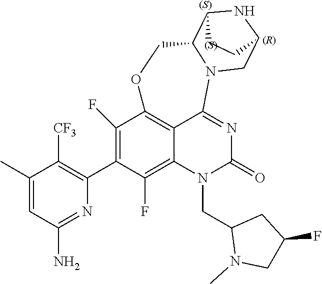 | (5aS,6S,9R)-2-(6-amino-4- methyl-3- (trifluoromethyl)pyridin-2-yl)- 1,3-difluoro-14-(((4R)-4-fluoro- 1-methylpyrrolidin-2- yl)methyl)-5a,6,7,8,9,10- hexahydro-5H-6,9- epiminoazepino[2′,1′:3,4][1,4] oxazepino[5,6,7-de]quinazolin- 13(14H)-one | 610 [M + H]⁺ |
| 196 | 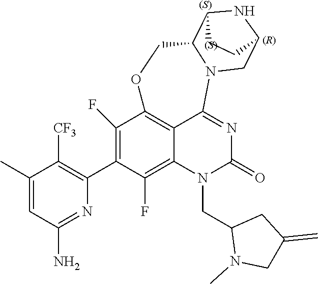 | (5aS,6S,9R)-2-(6-amino-4- methyl-3- (trifluoromethyl)pyridin-2-yl)- 1,3-difluoro-14-((1-methyl-4- methylenepyrrolidin-2- yl)methyl)-5a,6,7,8,9,10- hexahydro-5H-6,9- epiminoazepino[2′,1′:3,4][1,4] oxazepino[5,6,7-de]quinazolin- 13(14H)-one | 604 [M + H]⁺ |
| 197 | 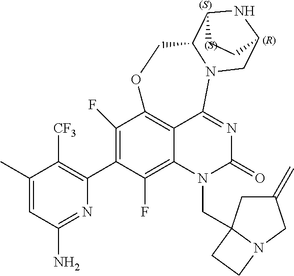 | (5aS,6S,9R)-2-(6-amino-4- methyl-3- (trifluoromethyl)pyridin-2-yl)- 1,3-difluoro-14-((3-methylene- 1-azabicyclo[3.2.0]heptan-5- yl)methyl)-5a,6,7,8,9,10- hexahydro-5H-6,9- epiminoazepino[2′,1′:3,4][1,4] oxazepino[5,6,7-de]quinazolin- 13(14H)-one | 616 [M + H]⁺ |
| 198 | 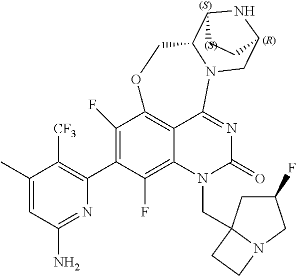 | (5aS,6S,9R)-2-(6-amino-4- methyl-3- (trifluoromethyl)pyridin-2-yl)- 1,3-difluoro-14-(((3R)-3-fluoro- 1-azabicyclo[3.2.0]heptan-5- yl)methyl)-5a,6,7,8,9,10- hexahydro-5H-6,9- epiminoazepino[2′,1′:3,4][1,4] oxazepino[5,6,7-de]quinazolin- 13(14H)-one | 622 [M + H]⁺ |
| 200a | 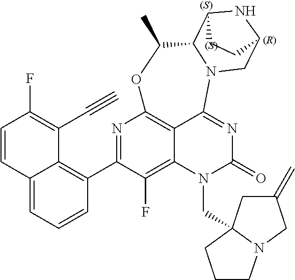 | (5S,5aS,6S,9R)-2-(8-ethynyl-7- fluoronaphthalen-1-yl)-1-fluoro- 5-methyl-13-(((S)-2- methylenetetrahydro-1H- pyrrolizin-7a(5H)-yl)methyl)- 5a,6,7,8,9,10-hexahydro-5H-4- oxa-3,10a,11,13,14-pentaaza- 6,9-methanonaphtho[1,8- ab]heptalen-12(13H)-one Isomer 1 (the first eluting isomer) | 621 [M + H]⁺ |
| 200b | 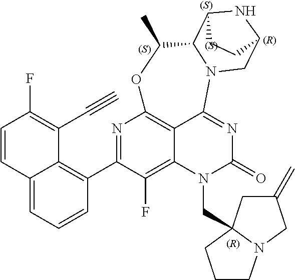 | (5S,5aS,6S,9R)-2-(8-ethynyl-7- fluoronaphthalen-1-yl)-1-fluoro- 5-methyl-13-(((R)-2- methylenetetrahydro-1H- pyrrolizin-7a(5H)-yl)methyl)- 5a,6,7,8,9,10-hexahydro-5H-4- oxa-3,10a,11,13,14-pentaaza- 6,9-methanonaphtho[1,8- ab]heptalen-12(13H)-one Isomer 2 (the second eluting isomer) | 621 [M + H]⁺ |
| 201 | 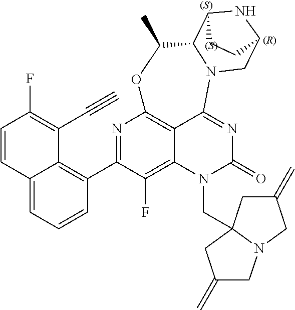 | (5S,5aS,6S,9R)-2-(8-ethynyl-7- fluoronaphthalen-1-yl)-1-fluoro- 13-(((2R,7aS)-2- fluorotetrahydro-1H-pyrrolizin- 7a(5H)-yl)methyl)-5-methyl- 5a,6,7,8,9,10-hexahydro-5H-4- oxa-3,10a,11,13,14-pentaaza- 6,9-methanonaphtho[1,8- ab]heptalen-12(13H)-one | 633 [M + H]⁺ |
| 202a | 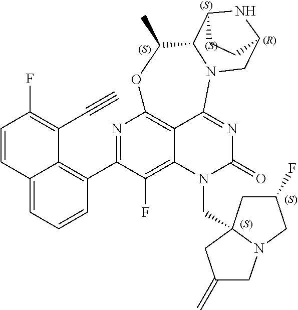 | (5S,5aS,6S,9R)-2-(8-ethynyl-7- fluoronaphthalen-1-yl)-1-fluoro- 13-(((2S,7aS)-2-fluoro-6- methylenetetrahydro-1H- pyrrolizin-7a(5H)-yl)methyl)-5- methyl-5a,6,7,8,9,10-hexahydro- 5H-4-oxa-3,10a,11,13,14- pentaaza-6,9- methanonaphtho[1,8- ab]heptalen-12(13H)-one Isomer 1 (the first eluting isomer) | 639 [M + H]⁺ |
| 202b | 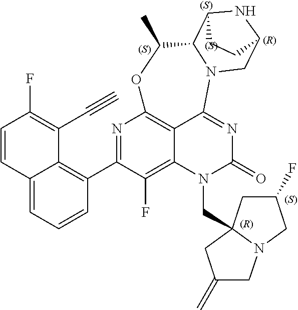 | (5S,5aS,6S,9R)-2-(8-ethynyl-7- fluoronaphthalen-1-yl)-1-fluoro- 13-(((2S,7aR)-2-fluoro-6- methylenetetrahydro-1H- pyrrolizin-7a(5H)-yl)methyl)-5- methyl-5a,6,7,8,9,10-hexahydro- 5H-4-oxa-3,10a,11,13,14- pentaaza-6,9- methanonaphtho[1,8- ab]heptalen-12(13H)-one Isomer 2 (the second eluting isomer) | 639 [M + H]⁺ |
| 203a | 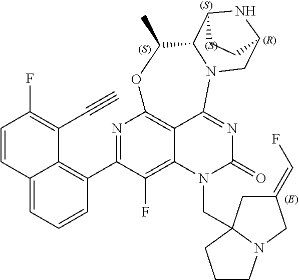 | (5S,5aS,6S,9R)-2-(8-ethynyl-7- fluoronaphthalen-1-yl)-1-fluoro- 13-((2-fluoro-6- methylenetetrahydro-1H- pyrrolizin-7a(5H)-yl)methyl)-5- methyl-5a,6,7,8,9,10-hexahydro- 5H-4-oxa-3,10a,11,13,14- pentaaza-6,9- methanonaphtho[1,8- ab]heptalen-12(13H)-one Isomer 1 (the first eluting isomer) | 639 [M + H]⁺ |
| 203b | 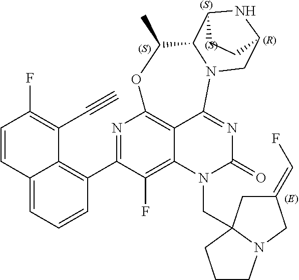 | (5S,5aS,6S,9R)-2-(8-ethynyl-7- fluoronaphthalen-1-yl)-1-fluoro- 13-(((E)-2- (fluoromethylene)tetrahydro- 1H-pyrrolizin-7a(5H)- yl)methyl)-5-methyl- 5a,6,7,8,9,10-hexahydro-5H-4- oxa-3,10a,11,13,14-pentaaza- 6,9-methanonaphtho[1,8- ab]heptalen-12(13H)-one Isomer 2 (the second eluting isomer) | 639 [M + H]⁺ |
| 204 | 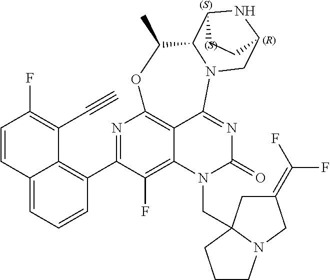 | (5S,5aS,6S,9R)-13-((2- (difluoromethylene)tetrahydro- 1H-pyrrolizin-7a(5H)- yl)methyl)-2-(8-ethynyl-7- fluoronaphthalen-1-yl)-1-fluoro- 5-methyl-5a,6,7,8,9,10- hexahydro-5H-4-oxa- 3,10a,11,13,14-pentaaza-6,9- methanonaphtho[1,8- ab]heptalen-12(13H)-one | 657 [M + H]⁺ |
| 205 | 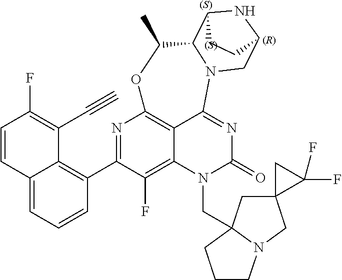 | (5S,5aS,6S,9R)-13-((2,2- difluorodihydro-1′H,3′H- spiro[cyclopropane-1,2′- pyrrolizin]-7a′(5′H)-yl)methyl)- 2-(8-ethynyl-7- fluoronaphthalen-1-yl)-1-fluoro- 5-methyl-5a,6,7,8,9,10- hexahydro-5H-4-oxa- 3,10a,11,13,14-pentaaza-6,9- methanonaphtho[1,8- ab]heptalen-12(13H)-one | 671 [M + H]⁺ |
| 206 | 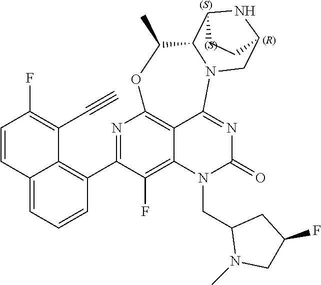 | (5S,5aS,6S,9R)-2-(8-ethynyl-7- fluoronaphthalen-1-yl)-1-fluoro- 13-(((4R)-4-fluoro-1- methylpyrrolidin-2-yl)methyl)- 5-methyl-5a,6,7,8,9,10- hexahydro-5H-4-oxa- 3,10a,11,13,14-pentaaza-6,9- methanonaphtho[1,8- ab]heptalen-12(13H)-one | 601 [M + H]⁺ |
| 207 | 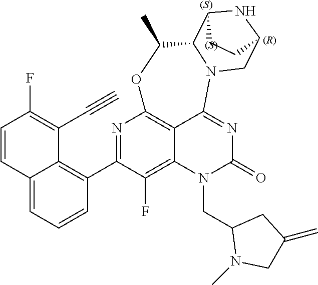 | (5S,5aS,6S,9R)-2-(8-ethynyl-7- fluoronaphthalen-1-yl)-1-fluoro- 5-methyl-13-((1-methyl-4- methylenepyrrolidin-2- yl)methyl)-5a,6,7,8,9,10- hexahydro-5H-4-oxa- 3,10a,11,13,14-pentaaza-6,9- methanonaphtho[1,8- ab]heptalen-12(13H)-one | 595 [M + H]⁺ |
| 208 | 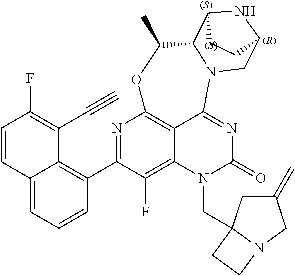 | (5S,5aS,6S,9R)-2-(8-ethynyl-7- fluoronaphthalen-1-yl)-1-fluoro- 5-methyl-13-((3-methylene-1- azabicyclo[3.2.0]heptan-5- yl)methyl)-5a,6,7,8,9,10- hexahydro-5H-4-oxa- 3,10a,11,13,14-pentaaza-6,9- methanonaphtho[1,8- ab]heptalen-12(13H)-one | 607 [M + H]⁺ |
| 209 | 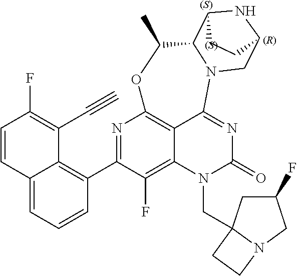 | (5S,5aS,6S,9R)-2-(8-ethynyl-7- fluoronaphthalen-1-yl)-1-fluoro- 13-(((3R)-3-fluoro-1- azabicyclo[3.2.0]heptan-5- yl)methyl)-5-methyl- 5a,6,7,8,9,10-hexahydro-5H-4- oxa-3,10a,11,13,14-pentaaza- 6,9-methanonaphtho[1,8- ab]heptalen-12(13H)-one | 613 [M + H]⁺ |
| 210 | 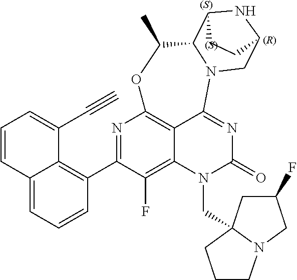 | (5S,5aS,6S,9R)-2-(8- ethynylnaphthalen-1-yl)-1- fluoro-5-methyl-13-((2- methylenetetrahydro-1H- pyrrolizin-7a(5H)-yl)methyl)- 5a,6,7,8,9,10-hexahydro-5H-4- oxa-3,10a,11,13,14-pentaaza- 6,9-methanonaphtho[1,8- ab]heptalen-12(13H)-one | 609 [M + H]⁺ |
| 211 | 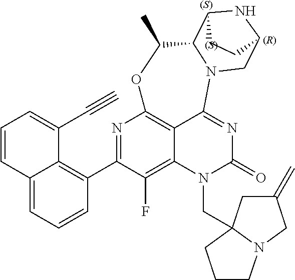 | (5S,5aS,6S,9R)-13-((2,6- dimethylenetetrahydro-1H- pyrrolizin-7a(5H)-yl)methyl)-2- (8-ethynylnaphthalen-1-yl)-1- fluoro-5-methyl-5a,6,7,8,9,10- hexahydro-5H-4-oxa- 3,10a,11,13,14-pentaaza-6,9- methanonaphtho[1,8- ab]heptalen-12(13H)-one | 603 [M + H]⁺ |
| 212 | 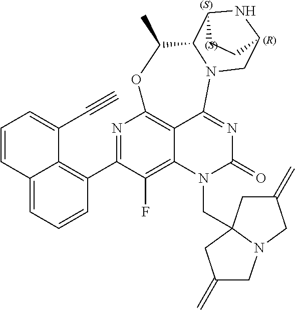 | (5S,5aS,6S,9R)-2-(8- ethynylnaphthalen-1-yl)-1- fluoro-13-(((2R,7aS)-2- fluorotetrahydro-1H-pyrrolizin- 7a(5H)-yl)methyl)-5-methyl- 5a,6,7,8,9,10-hexahydro-5H-4- oxa-3,10a,11,13,14-pentaaza- 6,9-methanonaphtho[1,8- ab]heptalen-12(13H)-one | 615 [M + H]⁺ |
| 213 | 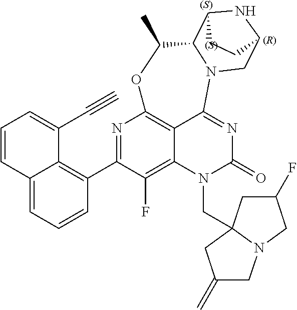 | (5S,5aS,6S,9R)-2-(8- ethynylnaphthalen-1-yl)-1- fluoro-13-((2- (fluoromethylene)tetrahydro- 1H-pyrrolizin-7a(5H)- yl)methyl)-5-methyl- 5a,6,7,8,9,10-hexahydro-5H-4- oxa-3,10a,11,13,14-pentaaza- 6,9-methanonaphtho[1,8- ab]heptalen-12(13H)-one | 621 [M + H]⁺ |
| 214 | 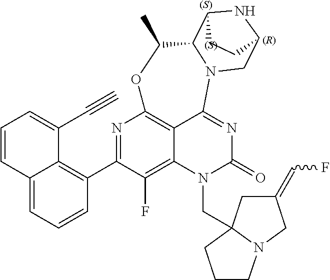 | (5S,5aS,6S,9R)-2-(8- ethynylnaphthalen-1-yl)-1- fluoro-13-((2-fluoro-6- methylenetetrahydro-1H- pyrrolizin-7a(5H)-yl)methyl)-5- methyl-5a,6,7,8,9,10-hexahydro- 5H-4-oxa-3,10a,11,13,14- pentaaza-6,9- methanonaphtho[1,8- ab]heptalen-12(13H)-one | 621 [M + H]⁺ |
| 215 | 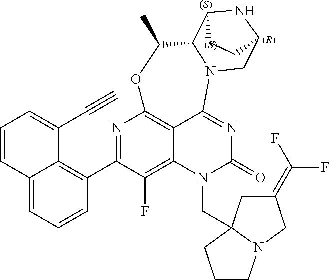 | (5S,5aS,6S,9R)-13-((2- (difluoromethylene)tetrahydro- 1H-pyrrolizin-7a(5H)- yl)methyl)-2-(8- ethynylnaphthalen-1-yl)-1- fluoro-5-methyl-5a,6,7,8,9,10- hexahydro-5H-4-oxa- 3,10a,11,13,14-pentaaza-6,9- methanonaphtho[1,8- ab]heptalen-12(13H)-one | 639 [M + H]⁺ |
| 216 | 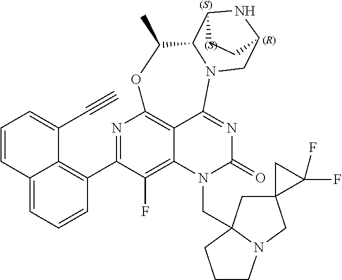 | (5S,5aS,6S,9R)-13-((2,2- difluorodihydro-1′H,3′H- spiro[cyclopropane-1,2′- pyrrolizin]-7a′(5′H)-yl)methyl)- 2-(8-ethynylnaphthalen-1-yl)-1- fluoro-5-methyl-5a,6,7,8,9,10- hexahydro-5H-4-oxa- 3,10a,11,13,14-pentaaza-6,9- methanonaphtho[1,8- ab]heptalen-12(13H)-one | 653 [M + H]⁺ |
| 217 | 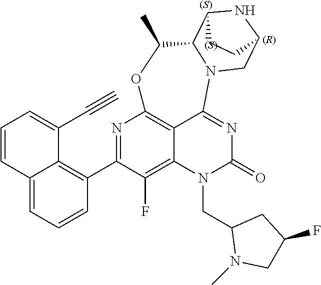 | (5S,5aS,6S,9R)-2-(8- ethynylnaphthalen-1-yl)-1- fluoro-13-(((4R)-4-fluoro-1- methylpyrrolidin-2-yl)methyl)- 5-methyl-5a,6,7,8,9,10- hexahydro-5H-4-oxa- 3,10a,11,13,14-pentaaza-6,9- methanonaphtho[1,8- ab]heptalen-12(13H)-one | 583 [M + H]⁺ |
| 218 | 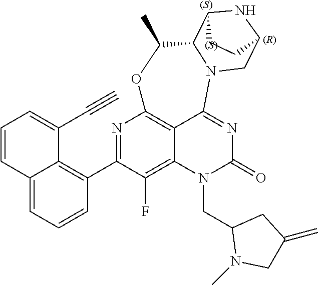 | (5S,5aS,6S,9R)-2-(8- ethynylnaphthalen-1-yl)-1- fluoro-5-methyl-13-((1-methyl- 4-methylenepyrrolidin-2- yl)methyl)-5a,6,7,8,9,10- hexahydro-5H-4-oxa- 3,10a,11,13,14-pentaaza-6,9- methanonaphtho[1,8- ab]heptalen-12(13H)-one | 577 [M + H]⁺ |
| 219 | 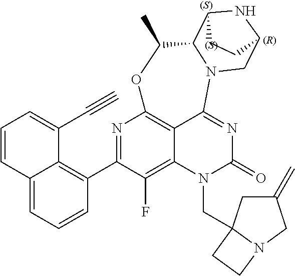 | (5S,5aS,6S,9R)-2-(8- ethynylnaphthalen-1-yl)-1- fluoro-5-methyl-13-((3- methylene-1- azabicyclo[3.2.0]heptan-5- yl)methyl)-5a,6,7,8,9,10- hexahydro-5H-4-oxa- 3,10a,11,13,14-pentaaza-6,9- methanonaphtho[1,8- ab]heptalen-12(13H)-one | 589 [M + H]⁺ |
| 220 | 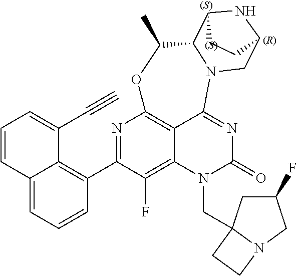 | (5S,5aS,6S,9R)-2-(8- ethynylnaphthalen-1-yl)-1- fluoro-13-(((3R)-3-fluoro-1- azabicyclo[3.2.0]heptan-5- yl)methyl)-5-methyl- 5a,6,7,8,9,10-hexahydro-5H-4- oxa-3,10a,11,13,14-pentaaza- 6,9-methanonaphtho[1,8- ab]heptalen-12(13H)-one | 595 [M + H]⁺ |
| 221 | 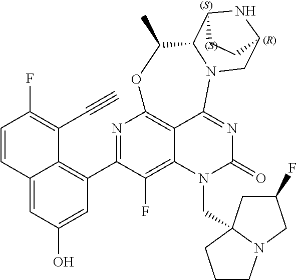 | (5S,5aS,6S,9R)-2-(8-ethynyl-7- fluoro-3-hydroxynaphthalen-1- yl)-1-fluoro-5-methyl-13-((2- methylenetetrahydro-1H- pyrrolizin-7a(5H)-yl)methyl)- 5a,6,7,8,9,10-hexahydro-5H-4- oxa-3,10a,11,13,14-pentaaza- 6,9-methanonaphtho[1,8- ab]heptalen-12(13H)-one | 643 [M + H]⁺ |
| 222 | 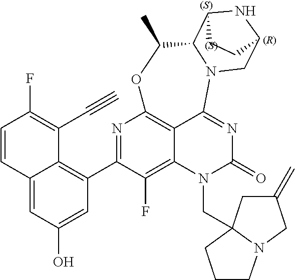 | (5S,5aS,6S,9R)-13-((2,6- dimethylenetetrahydro-1H- pyrrolizin-7a(5H)-yl)methyl)-2- (8-ethynyl-7-fluoro-3- hydroxynaphthalen-1-yl)-1- fluoro-5-methyl-5a,6,7,8,9,10- hexahydro-5H-4-oxa- 3,10a,11,13,14-pentaaza-6,9- methanonaphtho[1,8- ab]heptalen-12(13H)-one | 637 [M + H]⁺ |
| 223 | 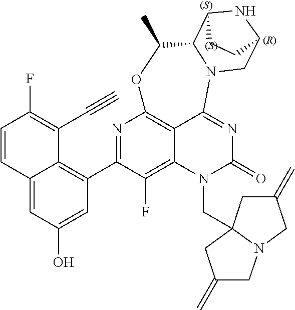 | (5S,5aS,6S,9R)-2-(8-ethynyl-7- fluoro-3-hydroxynaphthalen-1- yl)-1-fluoro-13-(((2R,7aS)-2- fluorotetrahydro-1H-pyrrolizin- 7a(5H)-yl)methyl)-5-methyl- 5a,6,7,8,9,10-hexahydro-5H-4- oxa-3,10a,11,13,14-pentaaza- 6,9-methanonaphtho[1,8- ab]heptalen-12(13H)-one | 649 [M + H]⁺ |
| 224 | 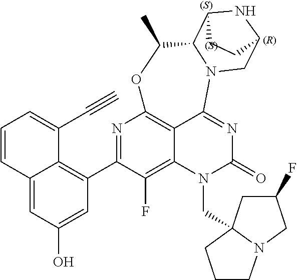 | (5S,5aS,6S,9R)-2-(8-ethynyl-3- hydroxynaphthalen-1-yl)-1- fluoro-5-methyl-13-((2- methylenetetrahydro-1H- pyrrolizin-7a(5H)-yl)methyl)- 5a,6,7,8,9,10-hexahydro-5H-4- oxa-3,10a,11,13,14-pentaaza- 6,9-methanonaphtho[1,8- ab]heptalen-12(13H)-one | 625 [M + H]⁺ |
| 225 | 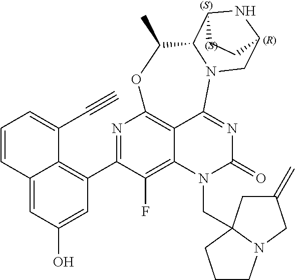 | (5S,5aS,6S,9R)-13-((2,6- dimethylenetetrahydro-1H- pyrrolizin-7a(5H)-yl)methyl)-2- (8-ethynyl-3- hydroxynaphthalen-1-yl)-1- fluoro-5-methyl-5a,6,7,8,9,10- hexahydro-5H-4-oxa- 3,10a,11,13,14-pentaaza-6,9- methanonaphtho[1,8- ab]heptalen-12(13H)-one | 619 [M + H]⁺ |
| 226 | 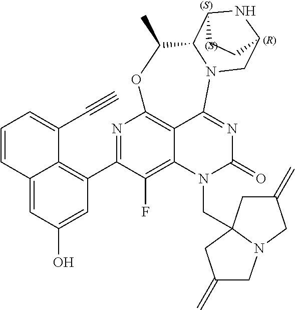 | (5S,5aS,6S,9R)-2-(8-ethynyl-3- hydroxynaphthalen-1-yl)-1- fluoro-13-(((2R,7aS)-2- fluorotetrahydro-1H-pyrrolizin- 7a(5H)-yl)methyl)-5-methyl- 5a,6,7,8,9,10-hexahydro-5H-4- oxa-3,10a,11,13,14-pentaaza- 6,9-methanonaphtho[1,8- ab]heptalen-12(13H)-one | 631 [M + H]⁺ |
| 227 | 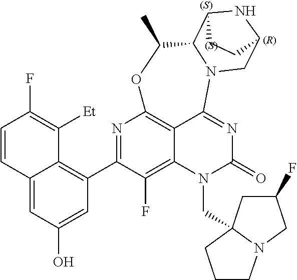 | (5S,5aS,6S,9R)-2-(8-ethyl-7- fluoro-3-hydroxynaphthalen-1- yl)-1-fluoro-5-methyl-13-((2- methylenetetrahydro-1H- pyrrolizin-7a(5H)-yl)methyl)- 5a,6,7,8,9,10-hexahydro-5H-4- oxa-3,10a,11,13,14-pentaaza- 6,9-methanonaphtho[1,8- ab]heptalen-12(13H)-one | 647 [M + H]⁺ |
| 228 | 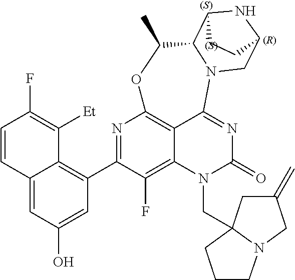 | (5S,5aS,6S,9R)-13-((2,6- dimethylenetetrahydro-1H- pyrrolizin-7a(5H)-yl)methyl)-2- (8-ethyl-7-fluoro-3- hydroxynaphthalen-1-yl)-1- fluoro-5-methyl-5a,6,7,8,9,10- hexahydro-5H-4-oxa- 3,10a,11,13,14-pentaaza-6,9- methanonaphtho[1,8- ab]heptalen-12(13H)-one | 641 [M + H]⁺ |
| 229 | 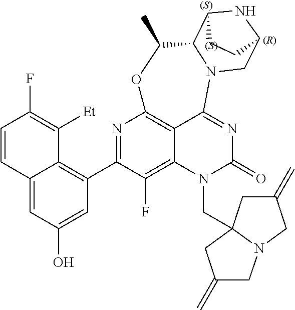 | (5S,5aS,6S,9R)-2-(8-ethyl-7- fluoro-3-hydroxynaphthalen-1- yl)-1-fluoro-13-(((2R,7aS)-2- fluorotetrahydro-1H-pyrrolizin- 7a(5H)-yl)methyl)-5-methyl- 5a,6,7,8,9,10-hexahydro-5H-4- oxa-3,10a,11,13,14-pentaaza- 6,9-methanonaphtho[1,8- ab]heptalen-12(13H)-one | 653 [M + H]⁺ |
| 230 | 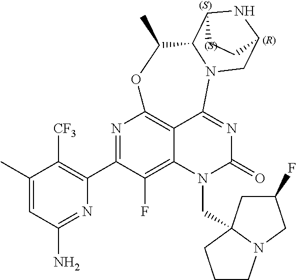 | (5S,5aS,6S,9R)-2-(6-amino-4- methyl-3- (trifluoromethyl)pyridin-2-yl)-1- fluoro-5-methyl-13-((2- methylenetetrahydro-1H- pyrrolizin-7a(5H)-yl)methyl)- 5a,6,7,8,9,10-hexahydro-5H-4- oxa-3,10a,11,13,14-pentaaza- 6,9-methanonaphtho[1,8- ab]heptalen-12(13H)-one | 633 [M + H]⁺ |
| 231 | 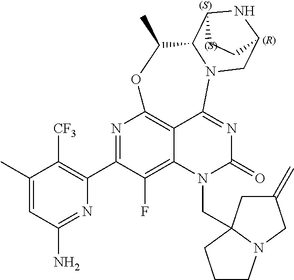 | (5S,5aS,6S,9R)-2-(6-amino-4- methyl-3- (trifluoromethyl)pyridin-2-yl)- 13-((2,6-dimethylenetetrahydro- 1H-pyrrolizin-7a(5H)- yl)methyl)-1-fluoro-5-methyl- 5a,6,7,8,9,10-hexahydro-5H-4- oxa-3,10a,11,13,14-pentaaza- 6,9-methanonaphtho[1,8- ab]heptalen-12(13H)-one | 627 [M + H]⁺ |
| 232 | 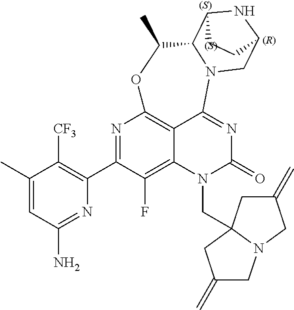 | (5S,5aS,6S,9R)-2-(6-amino-4- methyl-3- (trifluoromethyl)pyridin-2-yl)-1- fluoro-13-(((2R,7aS)-2- fluorotetrahydro-1H-pyrrolizin- 7a(5H)-yl)methyl)-5-methyl- 5a,6,7,8,9,10-hexahydro-5H-4- oxa-3,10a,11,13,14-pentaaza- 6,9-methanonaphtho[1,8- ab]heptalen-12(13H)-one | 639 [M + H]⁺ |
| 233 | 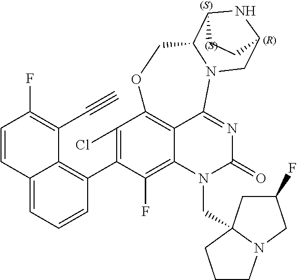 | (5aS,6S,9R)-3-chloro-2-(8- ethynyl-7-fluoronaphthalen-1- yl)-1-fluoro-14-((2- methylenetetrahydro-1H- pyrrolizin-7a(5H)-yl)methyl)- 5a,6,7,8,9,10-hexahydro-5H-6,9- epiminoazepino[2′,1′:3,4][1,4] oxazepino[5,6,7-de]quinazolin- 13(14H)-one | 646 [M + H]⁺ |
| 234 | 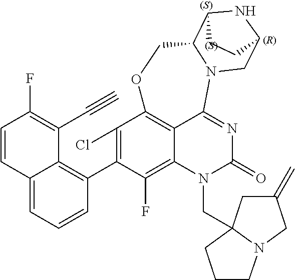 | (5aS,6S,9R)-3-chloro-14-((2,6- dimethylenetetrahydro-1H- pyrrolizin-7a(5H)-yl)methyl)-2- (8-ethynyl-7-fluoronaphthalen- 1-yl)-1-fluoro-5a,6,7,8,9,10- hexahydro-5H-6,9- epiminoazepino[2′,1′:3,4][1,4] oxazepino[5,6,7-de]quinazolin- 13(14H)-one | 640 [M + H]⁺ |
| 235 | 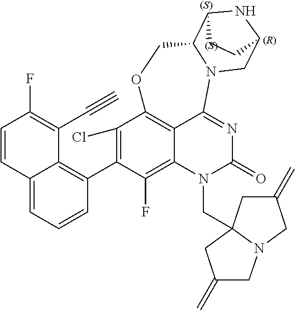 | (5aS,6S,9R)-3-chloro-2-(8- ethynyl-7-fluoronaphthalen-1- yl)-1-fluoro-14-(((2R,7aS)-2- fluorotetrahydro-1H-pyrrolizin- 7a(5H)-yl)methyl)-5a,6,7,8,9,10- hexahydro-5H-6,9- epiminoazepino[2′,1′:3,4][1,4] oxazepino[5,6,7-de]quinazolin- 13(14H)-one | 652 [M + H]⁺ |
| 236 | 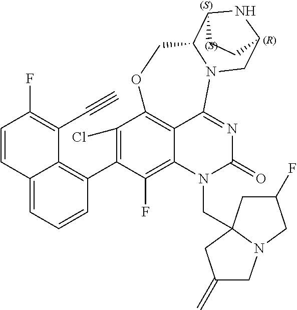 | (5aS,6S,9R)-3-chloro-2-(8- ethynyl-7-fluoronaphthalen-1- yl)-1-fluoro-14-((2- (fluoromethylene)tetrahydro- 1H-pyrrolizin-7a(5H)- yl)methyl)-5a,6,7,8,9,10- hexahydro-5H-6,9- epiminoazepino[2′,1′:3,4][1,4] oxazepino[5,6,7-de]quinazolin- 13(14H)-one | 658 [M + H]⁺ |
| 237 | 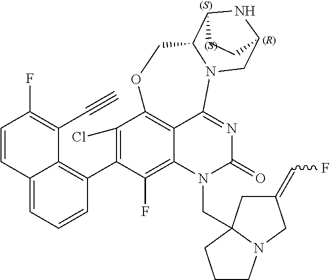 | (5aS,6S,9R)-3-chloro-2-(8- ethynyl-7-fluoronaphthalen-1- yl)-1-fluoro-14-((2-fluoro-6- methylenetetrahydro-1H- pyrrolizin-7a(5H)-yl)methyl)- 5a,6,7,8,9,10-hexahydro-5H-6,9- epiminoazepino[2′,1′:3,4][1,4] oxazepino[5,6,7-de]quinazolin- 13(14H)-one | 658 [M + H]⁺ |
| 238 | 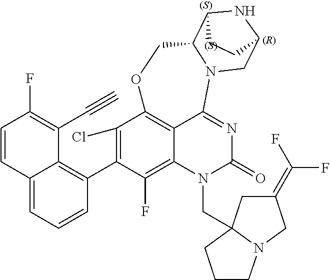 | (5aS,6S,9R)-3-chloro-14-((2- (difluoromethylene)tetrahydro- 1H-pyrrolizin-7a(5H)- yl)methyl)-2-(8-ethynyl-7- fluoronaphthalen-1-yl)-1-fluoro- 5a,6,7,8,9,10-hexahydro-5H-6,9- epiminoazepino[2′,1′:3,4][1,4] oxazepino[5,6,7-de]quinazolin- 13(14H)-one | 676 [M + H]⁺ |
| 239 | 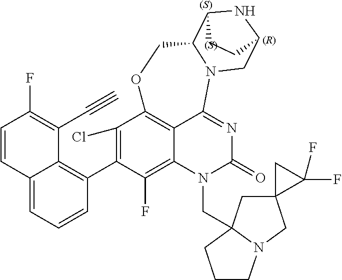 | (5aS,6S,9R)-3-chloro-14-((2,2- difluorodihydro-1′H,3′H- spiro[cyclopropane-1,2′- pyrrolizin]-7a′(5′H)-yl)methyl)- 2-(8-ethynyl-7- fluoronaphthalen-1-yl)-1-fluoro- 5a,6,7,8,9,10-hexahydro-5H-6,9- epiminoazepino[2′,1′:3,4][1,4] oxazepino[5,6,7-de]quinazolin- 13(14H)-one | 690 [M + H]⁺ |
| 240 | 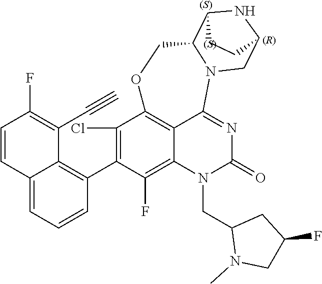 | (5aS,6S,9R)-3-chloro-2-(8- ethynyl-7-fluoronaphthalen-1- yl)-1-fluoro-14-(((4R)-4-fluoro- 1-methylpyrrolidin-2- yl)methyl)-5a,6,7,8,9,10- hexahydro-5H-6,9- epiminoazepino[2′,1′:3,4][1,4] oxazepino[5,6,7-de]quinazolin- 13(14H)-one | 620 [M + H]⁺ |
| 241 | 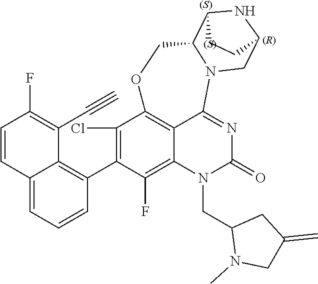 | (5aS,6S,9R)-3-chloro-2-(8- ethynyl-7-fluoronaphthalen-1- yl)-1-fluoro-14-((1-methyl-4- methylenepyrrolidin-2- yl)methyl)-5a,6,7,8,9,10- hexahydro-5H-6,9- epiminoazepino[2′,1′:3,4][1,4] oxazepino[5,6,7-de]quinazolin- 13(14H)-one | 614 [M + H]⁺ |
| 242 | 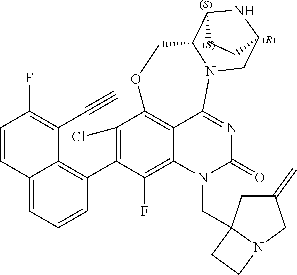 | (5aS,6S,9R)-3-chloro-2-(8- ethynyl-7-fluoronaphthalen-1- yl)-1-fluoro-14-((3-methylene-1- azabicyclo[3.2.0]heptan-5- yl)methyl)-5a,6,7,8,9,10- hexahydro-5H-6,9- epiminoazepino[2′,1′:3,4][1,4] oxazepino[5,6,7-de]quinazolin- 13(14H)-one | 626 [M + H]⁺ |
| 243 | 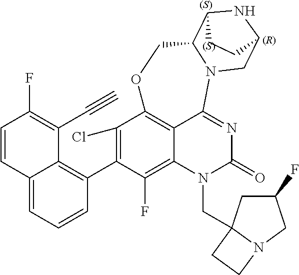 | (5aS,6S,9R)-3-chloro-2-(8- ethynyl-7-fluoronaphthalen-1- yl)-1-fluoro-14-(((3R)-3-fluoro- 1-azabicyclo[3.2.0]heptan-5- yl)methyl)-5a,6,7,8,9,10- hexahydro-5H-6,9- epiminoazepino[2′,1′:3,4][1,4] oxazepino[5,6,7-de]quinazolin- 13(14H)-one | 632 [M + H]⁺ |
| 244 | 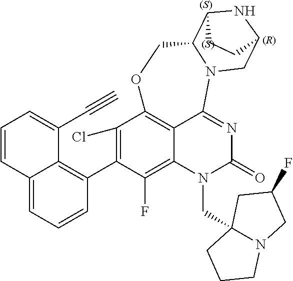 | (5aS,6S,9R)-3-chloro-2-(8- ethynylnaphthalen-1-yl)-1- fluoro-14-((2- methylenetetrahydro-1H- pyrrolizin-7a(5H)-yl)methyl)- 5a,6,7,8,9,10-hexahydro-5H-6,9- epiminoazepino[2′,1′:3,4][1,4] oxazepino[5,6,7-de]quinazolin- 13(14H)-one | 628 [M + H]⁺ |
| 245 | 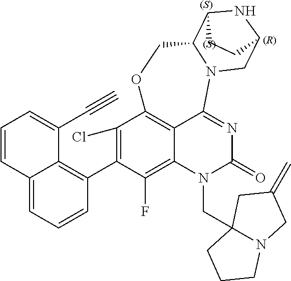 | (5aS,6S,9R)-3-chloro-14-((2,6- dimethylenetetrahydro-1H- pyrrolizin-7a(5H)-yl)methyl)-2- (8-ethynylnaphthalen-1-yl)-1- fluoro-5a,6,7,8,9,10-hexahydro- 5H-6,9- epiminoazepino[2′,1′:3,4][1,4] oxazepino[5,6,7-de]quinazolin- 13(14H)-one | 622 [M + H]⁺ |
| 246 | 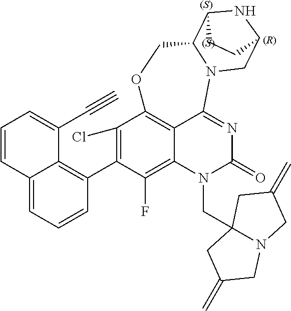 | (5aS,6S,9R)-3-chloro-2-(8- ethynylnaphthalen-1-yl)-1- fluoro-14-(((2R,7aS)-2- fluorotetrahydro-1 H-pyrrolizin- 7a(5H)-yl)methyl)-5a,6,7,8,9,10- hexahydro-5H-6,9- epiminoazepino[2′,1′:3,4][1,4] oxazepino[5,6,7-de]quinazolin- 13(14H)-one | 634 [M + H]⁺ |
| 247 | 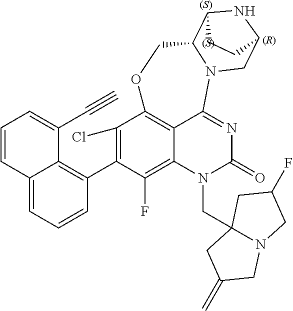 | (5aS,6S,9R)-3-chloro-2-(8- ethynylnaphthalen-1-yl)-1- fluoro-14-((2- (fluoromethylene)tetrahydro- 1H-pyrrolizin-7a(5H)- yl)methyl)-5a,6,7,8,9,10- hexahydro-5H-6,9- epiminoazepino[2′,1′:3,4][1,4] oxazepino[5,6,7-de]quinazolin- 13(14H)-one | 640 [M + H]⁺ |
| 248 | 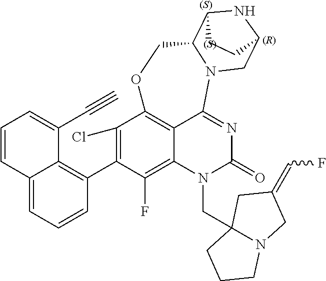 | (5aS,6S,9R)-3-chloro-2-(8- ethynylnaphthalen-1-yl)-1- fluoro-14-((2-fluoro-6- methylenetetrahydro-1H- pyrrolizin-7a(5H)-yl)methyl)- 5a,6,7,8,9,10-hexahydro-5H-6,9- epiminoazepino[2′,1′:3,4][1,4] oxazepino[5,6,7-de]quinazolin- 13(14H)-one | 640 [M + H]⁺ |
| 249 | 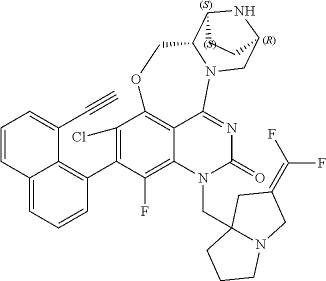 | (5aS,6S,9R)-3-chloro-14-((2- (difluoromethylene)tetrahydro- 1H-pyrrolizin-7a(5H)- yl)methyl)-2-(8- ethynylnaphthalen-1-yl)-1- fluoro-5a,6,7,8,9,10-hexahydro- 5H-6,9- epiminoazepino[2′,1′:3,4][1,4] oxazepino[5,6,7-de]quinazolin- 13(14H)-one | 658 [M + H]⁺ |
| 250 | 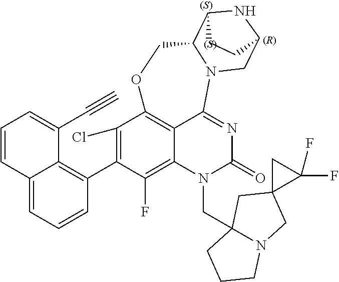 | (5aS,6S,9R)-3-chloro-14-((2,2- difluorodihydro-1′H,3′H- spiro[cyclopropane-1,2′- pyrrolizin]-7a′(5′H)-yl)methyl)- 2-(8-ethynylnaphthalen-1-yl)-1- fluoro-5a,6,7,8,9,10-hexahydro- 5H-6,9- epiminoazepino[2′,1′:3,4][1,4] oxazepino[5,6,7-de]quinazolin- 13(14H)-one | 672 [M + H]⁺ |
| 251 | 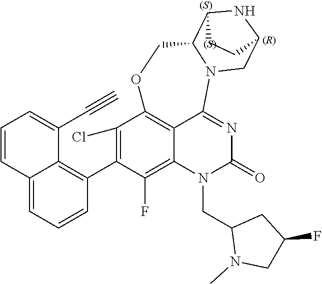 | (5aS,6S,9R)-3-chloro-2-(8- ethynylnaphthalen-1-yl)-1- fluoro-14-(((4R)-4-fluoro-1- methylpyrrolidin-2-yl)methyl)- 5a,6,7,8,9,10-hexahydro-5H-6,9- epiminoazepino[2′,1′:3,4][1,4] oxazepino[5,6,7-de]quinazolin- 13(14H)-one | 602 [M + H]⁺ |
| 252 | 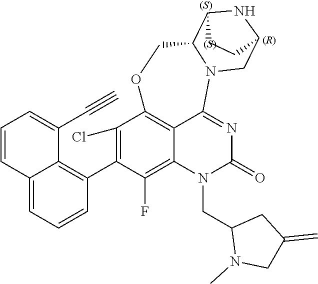 | (5aS,6S,9R)-3-chloro-2-(8- ethynylnaphthalen-1-yl)-1- fluoro-14-((1-methyl-4- methylenepyrrolidin-2- yl)methyl)-5a,6,7,8,9,10- hexahydro-5H-6,9- epiminoazepino[2′,1′:3,4][1,4] oxazepino[5,6,7-de]quinazolin- 13(14H)-one | 596 [M + H]⁺ |
| 253 | 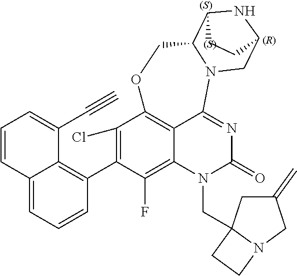 | (5aS,6S,9R)-3-chloro-2-(8- ethynylnaphthalen-1-yl)-1- fluoro-14-((3-methylene-1- azabicyclo[3.2.0]heptan-5- yl)methyl)-5a,6,7,8,9,10- hexahydro-5H-6,9- epiminoazepino[2′,1′:3,4][1,4] oxazepino[5,6,7-de]quinazolin- 13(14H)-one | 608 [M + H]⁺ |
| 254 | 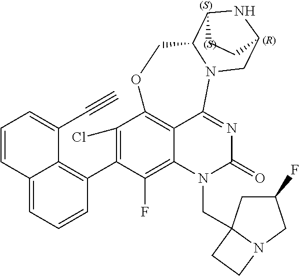 | (5aS,6S,9R)-3-chloro-2-(8- ethynylnaphthalen-1-yl)-1- fluoro-14-(((3R)-3-fluoro-1- azabicyclo[3.2.0]heptan-5- yl)methyl)-5a,6,7,8,9,10- hexahydro-5H-6,9- epiminoazepino[2′,1′:3,4][1,4] oxazepino[5,6,7-de]quinazolin- 13(14H)-one | 614 [M + H]⁺ |
| 255 | 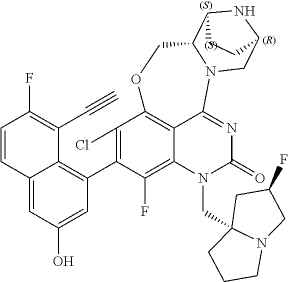 | (5aS,6S,9R)-3-chloro-2-(8- ethynyl-7-fluoro-3- hydroxynaphthalen-1-yl)-1- fluoro-14-((2- methylenetetrahydro-1H- pyrrolizin-7a(5H)-yl)methyl)- 5a,6,7,8,9,10-hexahydro-5H-6,9- epiminoazepino[2′,1′:3,4][1,4] oxazepino[5,6,7-de]quinazolin- 13(14H)-one | 662 [M + H]⁺ |
| 256 | 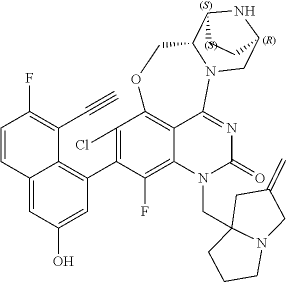 | (5aS,6S,9R)-3-chloro-14-((2,6- dimethylenetetrahydro-1H- pyrrolizin-7a(5H)-yl)methyl)-2- (8-ethynyl-7-fluoro-3- hydroxynaphthalen-1-yl)-1- fluoro-5a,6,7,8,9,10-hexahydro- 5H-6,9- epiminoazepino[2′,1′:3,4][1,4] oxazepino[5,6,7-de]quinazolin- 13(14H)-one | 656 [M + H]⁺ |
| 257 | 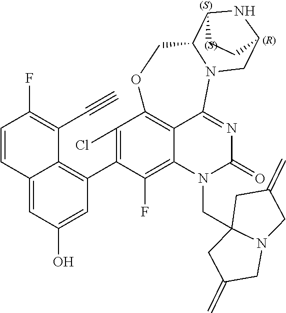 | (5aS,6S,9R)-3-chloro-2-(8- ethynyl-7-fluoro-3- hydroxynaphthalen-1-yl)-1- fluoro-14-(((2R,7aS)-2- fluorotetrahydro-1H-pyrrolizin- 7a(5H)-yl)methyl)-5a,6,7,8,9,10- hexahydro-5H-6,9- epiminoazepino[2′,1′:3,4][1,4] oxazepino[5,6,7-de]quinazolin- 13(14H)-one | 668 [M + H]⁺ |
| 258 | 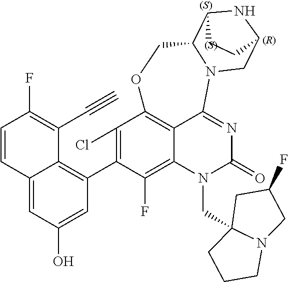 | (5aS,6S,9R)-3-chloro-2-(8- ethynyl-3-hydroxynaphthalen-1- yl)- 1-fluoro-14-((2- methylenetetrahydro-1H- pyrrolizin-7a(5H)-yl)methyl)- 5a,6,7,8,9,10-hexahydro-5H-6,9- epiminoazepino[2′,1′:3,4][1,4] oxazepino[5,6,7-de]quinazolin- 13(14H)-one | 644 [M + H]⁺ |
| 259 | 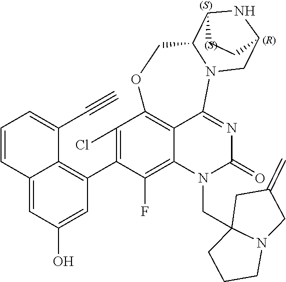 | (5aS,6S,9R)-3-chloro-14-((2,6- dimethylenetetrahydro-1H- pyrrolizin-7a(5H)-yl)methyl)-2- (8-ethynyl-3- hydroxynaphthalen-1-yl)-1- fluoro-5a,6,7,8,9,10-hexahydro- 5H-6,9- epiminoazepino[2′,1′:3,4][1,4] oxazepino[5,6,7-de]quinazolin- 13(14H)-one | 638 [M + H]⁺ |
| 260 | 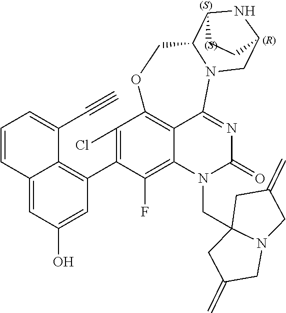 | (5aS,6S,9R)-3-chloro-2-(8- ethynyl-3-hydroxynaphthalen-1- yl)-1-fluoro-14-(((2R,7aS)-2- fluorotetrahydro-1H-pyrrolizin- 7a(5H)-yl)methyl)-5a,6,7,8,9,10- hexahydro-5H-6,9- epiminoazepino[2′,1′:3,4][1,4] oxazepino[5,6,7-de]quinazolin- 13(14H)-one | 650 [M + H]⁺ |
| 261 | 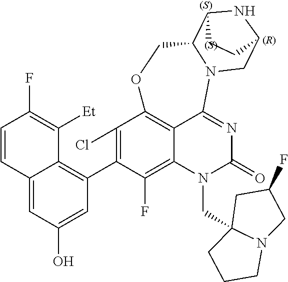 | (5aS,6S,9R)-3-chloro-2-(8- ethyl-7-fluoro-3- hydroxynaphthalen-1-yl)-1- fluoro-14-((2- methylenetetrahydro-1H- pyrrolizin-7a(5H)-yl)methyl)- 5a,6,7,8,9,10-hexahydro-5H-6,9- epiminoazepino[2′,1′:3,4][1,4] oxazepino[5,6,7-de]quinazolin- 13(14H)-one | 666 [M + H]⁺ |
| 262 | 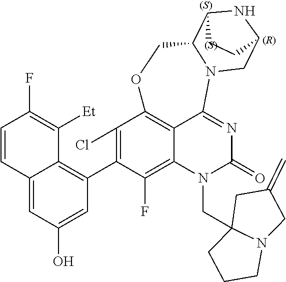 | (5aS,6S,9R)-3-chloro-2-(8- ethyl-7-fluoro-3- hydroxynaphthalen-1-yl)-1- fluoro-14-((2- methylenetetrahydro-1H- pyrrolizin-7a(5H)-yl)methyl)- 5a,6,7,8,9,10-hexahydro-5H-6,9- epiminoazepino[2′,1′:3,4][1,4] oxazepino[5,6,7-de]quinazolin- 13(14H)-one | 660 [M + H]⁺ |
| 263 | 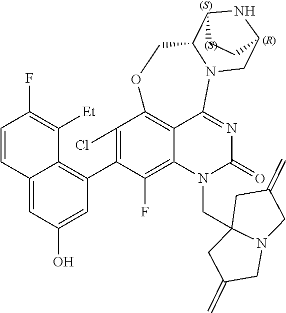 | (5aS,6S,9R)-3-chloro-2-(8- ethyl-7-fluoro-3- hydroxynaphthalen-1-yl)-1- fluoro-14-(((2R,7aS)-2- fluorotetrahydro-1H-pyrrolizin- 7a(5H)-yl)methyl)-5a,6,7,8,9,10- hexahydro-5H-6,9- epiminoazepino[2′,1′:3,4][1,4] oxazepino[5,6,7-de]quinazolin- 13(14H)-one | 672 [M + H]⁺ |
| 264 | 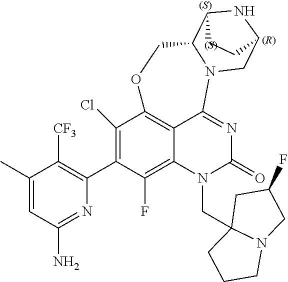 | (5aS,6S,9R)-2-(6-amino-4- methyl-3- (trifluoromethyl)pyridin-2-yl)-3- chloro-1-fluoro-14-((2- methylenetetrahydro-1H- pyrrolizin-7a(5H)-yl)methyl)- 5a,6,7,8,9,10-hexahydro-5H-6,9- epiminoazepino[2′,1′:3,4][1,4] oxazepino[5,6,7-de]quinazolin- 13(14H)-one | 652 [M + H]⁺ |
| 265 | 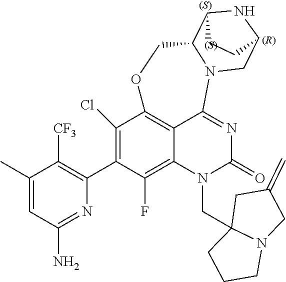 | (5aS,6S,9R)-2-(6-amino-4- methyl-3- (trifluoromethyl)pyridin-2-yl)-3- chloro-14-((2,6- dimethylenetetrahydro-1H- pyrrolizin-7a(5H)-yl)methyl)-1- fluoro-5a,6,7,8,9,10-hexahydro- 5H-6,9- epiminoazepino[2′,1′:3,4][1,4] oxazepino[5,6,7-de]quinazolin- 13(14H)-one | 646 [M + H]⁺ |
| 266 | 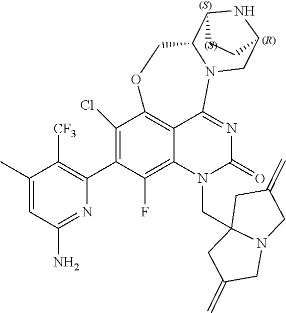 | (5aS,6S,9R)-2-(6-amino-4- methyl-3- (trifluoromethyl)pyridin-2-yl)-3- chloro-1-fluoro-14-(((2R,7aS)-2- fluorotetrahydro-1H-pyrrolizin- 7a(5H)-yl)methyl)-5a,6,7,8,9,10- hexahydro-5H-6,9- epiminoazepino[2′,1′:3,4][1,4] oxazepino[5,6,7-de]quinazolin- 13(14H)-one | 658 [M + H]⁺ |
| 267 | 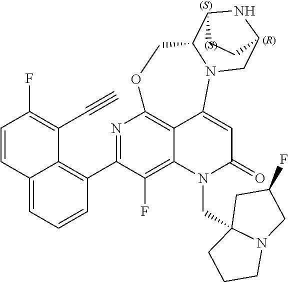 | (5aS,6S,9R)-2-(8-ethynyl-7- fluoronaphthalen-1-yl)-1-fluoro- 14-((2-methylenetetrahydro-1H- pyrrolizin-7a(5H)-yl)methyl)- 5a,6,7,8,9,10-hexahydro-5H-6,9- epiminoazepino[2′,1′:3,4][1,4] oxazepino[5,6,7- de][1,6]naphthyridin-13(14H)- one | 612 [M + H]⁺ |
| 268 | 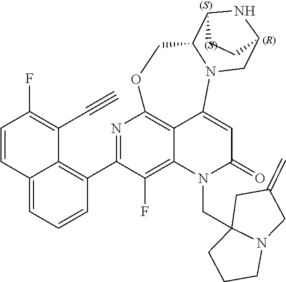 | (5aS,6S,9R)-14-((2,6- dimethylenetetrahydro-1H- pyrrolizin-7a(5H)-yl)methyl)-2- (8-ethynyl-7-fluoronaphthalen- 1-yl)-1-fluoro-5a,6,7,8,9,10- hexahydro-5H-6,9- epiminoazepino[2′,1′:3,4][1,4] oxazepino[5,6,7- de][1,6]naphthyridin-13(14H)- one | 606 [M + H]⁺ |
| 269 | 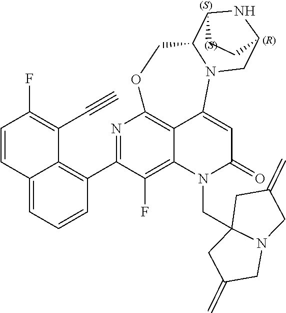 | (5aS,6S,9R)-2-(8-ethynyl-7- fluoronaphthalen-1-yl)-1-fluoro- 14-(((2R,7aS)-2- fluorotetrahydro-1H-pyrrolizin- 7a(5H)-yl)methyl)-5a,6,7,8,9,10- hexahydro-5H-6,9- epiminoazepino[2′,1′:3,4][1,4] oxazepino[5,6,7- de][1,6]naphthyridin-13(14H)- one | 618 [M + H]⁺ |
| 270 | 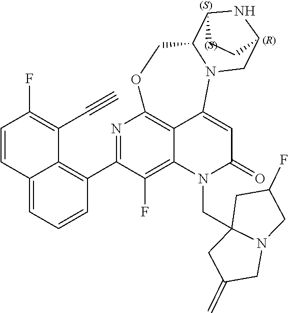 | (5aS,6S,9R)-2-(8-ethynyl-7- fluoronaphthalen-1-yl)-1-fluoro- 14-((2- (fluoromethylene)tetrahydro- 1H-pyrrolizin-7a(5H)- yl)methyl)-5a,6,7,8,9,10- hexahydro-5H-6,9- epiminoazepino[2′,1′:3,4][1,4] oxazepino[5,6,7- de][1,6]naphthyridin-13(14H)- one | 624 [M + H]⁺ |
| 271 | 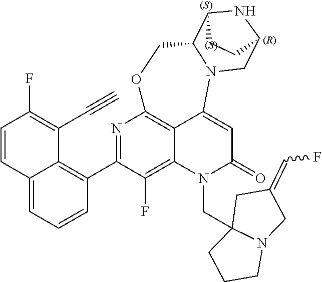 | (5aS,6S,9R)-2-(8-ethynyl-7- fluoronaphthalen-1-yl)-1-fluoro- 14-((2-fluoro-6- methylenetetrahydro-1H- pyrrolizin-7a(5H)-yl)methyl)- 5a,6,7,8,9,10-hexahydro-5H-6,9- epiminoazepino[2′,1′:3,4][1,4] oxazepino[5,6,7- de][1,6]naphthyridin-13(14H)- one | 624 [M + H]⁺ |
| 272 | 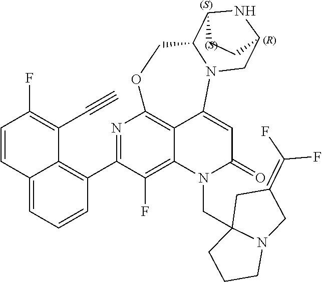 | (5aS,6S,9R)-14-((2- (difluoromethylene)tetrahydro- 1H-pyrrolizin-7a(5H)- yl)methyl)-2-(8-ethynyl-7- fluoronaphthalen-1-yl)-1-fluoro- 5a,6,7,8,9,10-hexahydro-5H-6,9- epiminoazepino[2′,1′:3,4][1,4] oxazepino[5,6,7- de][1,6]naphthyridin-13(14H)- one | 642 [M + H]⁺ |
| 273 | 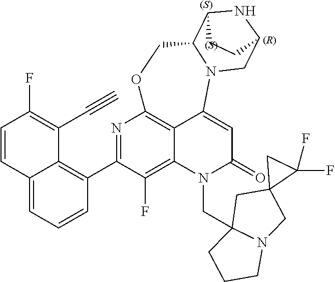 | (5aS,6S,9R)-14-((2,2- difluorodihydro-1′H,3′H- spiro[cyclopropane-1,2′- pyrrolizin]-7a′(5′H)-yl)methyl)- 2-(8-ethynyl-7- fluoronaphthalen-1-yl)-1-fluoro- 5a,6,7,8,9,10-hexahydro-5H-6,9- epiminoazepino[2′,1′:3,4][1,4] oxazepino[5,6,7- de][1,6]naphthyridin-13(14H)- one | 656 [M + H]⁺ |
| 274 | 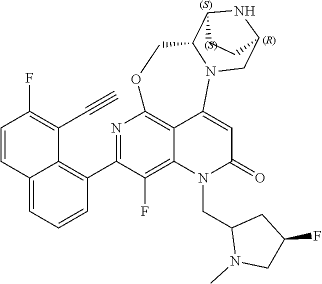 | (5aS,6S,9R)-2-(8-ethynyl-7- fluoronaphthalen-1-yl)-1-fluoro- 14-(((4R)-4-fluoro-1- methylpyrrolidin-2-yl)methyl)- 5a,6,7,8,9,10-hexahydro-5H-6,9- epiminoazepino[2′,1′:3,4][1,4] oxazepino[5,6,7- de][1,6]naphthyridin-13(14H)- one | 586 [M + H]⁺ |
| 275 | 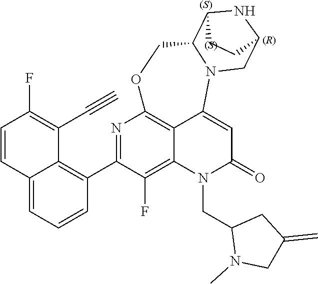 | (5aS,6S,9R)-2-(8-ethynyl-7- fluoronaphthalen-1-yl)-1-fluoro- 14-((1-methyl-4- methylenepyrrolidin-2- yl)methyl)-5a,6,7,8,9,10- hexahydro-5H-6,9- epiminoazepino[2′,1′:3,4][1,4] oxazepino[5,6,7- de][1,6]naphthyridin-13(14H)- one | 580 [M + H]⁺ |
| 276 | 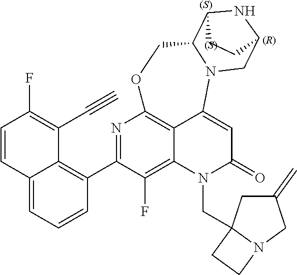 | (5aS,6S,9R)-2-(8-ethynyl-7- fluoronaphthalen-1-yl)-1-fluoro- 14-((3-methylene-1- azabicyclo[3.2.0]heptan-5- yl)methyl)-5a,6,7,8,9,10- hexahydro-5H-6,9- epiminoazepino[2′,1′:3,4][1,4] oxazepino[5,6,7- de][1,6]naphthyridin-13(14H)- one | 592 [M + H]⁺ |
| 277 | 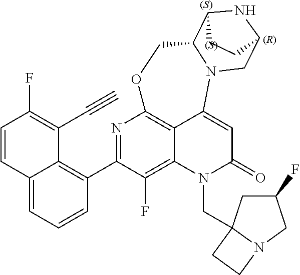 | (5aS,6S,9R)-2-(8-ethynyl-7- fluoronaphthalen-1-yl)-1-fluoro- 14-(((3R)-3-fluoro-1- azabicyclo[3.2.0]heptan-5- yl)methyl)-5a,6,7,8,9,10- hexahydro-5H-6,9- epiminoazepino[2′,1′:3,4][1,4] oxazepino[5,6,7- de][1,6]naphthyridin-13(14H)- one | 598 [M + H]⁺ |
| 278 | 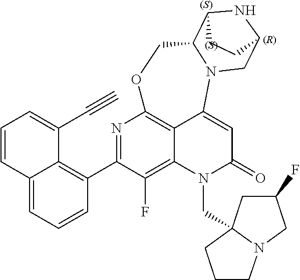 | (5aS,6S,9R)-2-(8- ethynylnaphthalen-1-yl)-1- fluoro-14-((2- methylenetetrahydro-1H- pyrrolizin-7a(5H)-yl)methyl)- 5a,6,7,8,9,10-hexahydro-5H-6,9- epiminoazepino[2′,1′:3,4][1,4] oxazepino[5,6,7- de][1,6]naphthyridin-13(14H)- one | 594 [M + H]⁺ |
| 279 | 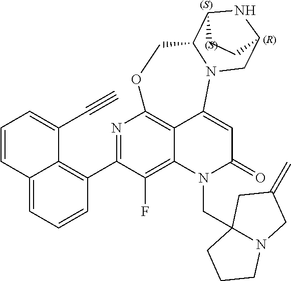 | (5aS,6S,9R)-14-((2,6- dimethylenetetrahydro-1H- pyrrolizin-7a(5H)-yl)methyl)-2- (8-ethynylnaphthalen-1-yl)-1- fluoro-5a,6,7,8,9,10-hexahydro- 5H-6,9- epiminoazepino[2′,1′:3,4][1,4] oxazepino[5,6,7- de][1,6]naphthyridin-13(14H)- one | 588 [M + H]⁺ |
| 280 | 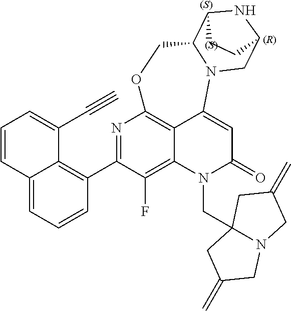 | (5aS,6S,9R)-2-(8- ethynylnaphthalen-1-yl)-1- fluoro-14-(((2R,7aS)-2- fluorotetrahydro-1H-pyrrolizin- 7a(5H)-yl)methyl)-5a,6,7,8,9,10- hexahydro-5H-6,9- epiminoazepino[2′,1′:3,4][1,4] oxazepino[5,6,7- de][1,6]naphthyridin-13(14H)- one | 600 [M + H]⁺ |
| 281 | 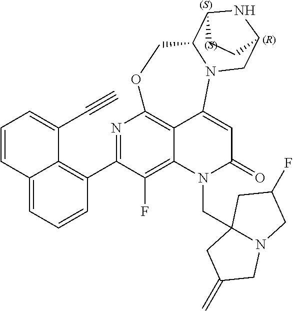 | (5aS,6S,9R)-2-(8- ethynylnaphthalen-1-yl)-1- fluoro-14-((2- (fluoromethylene)tetrahydro- 1H-pyrrolizin-7a(5H)- yl)methyl)-5a,6,7,8,9,10- hexahydro-5H-6,9- epiminoazepino[2′,1′:3,4][1,4] oxazepino[5,6,7- de][1,6]naphthyridin-13(14H)- one | 606 [M + H]⁺ |
| 282 | 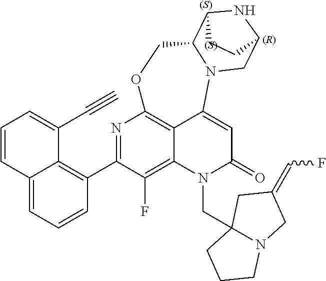 | (5aS,6S,9R)-2-(8- ethynylnaphthalen-1-yl)-1- fluoro-14-((2-fluoro-6- methylenetetrahydro-1H- pyrrolizin-7a(5H)-yl)methyl)- 5a,6,7,8,9,10-hexahydro-5H-6,9- epiminoazepino[2′,1′:3,4][1,4] oxazepino[5,6,7- de][1,6]naphthyridin-13(14H)- one | 606 [M + H]⁺ |
| 283 | 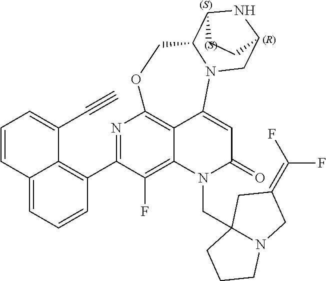 | (5aS,6S,9R)-14-((2- (difluoromethylene)tetrahydro- 1H-pyrrolizin-7a(5H)- yl)methyl)-2-(8- ethynylnaphthalen-1-yl)-1- fluoro-5a,6,7,8,9,10-hexahydro- 5H-6,9- epiminoazepino[2′,1′:3,4][1,4] oxazepino[5,6,7- de][1,6]naphthyridin-13(14H)- one | 624 [M + H]⁺ |
| 284 | 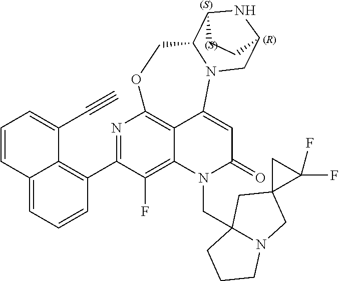 | (5aS,6S,9R)-14-((2,2- difluorodihydro-1′H,3′H- spiro[cyclopropane-1,2′- pyrrolizin]-7a′(5′H)-yl)methyl)- 2-(8-ethynylnaphthalen-1-yl)-1- fluoro-5a,6,7,8,9,10-hexahydro- 5H-6,9- epiminoazepino[2′,1′:3,4][1,4] oxazepino[5,6,7- de][1,6]naphthyridin-13(14H)- one | 638 [M + H]⁺ |
| 285 | 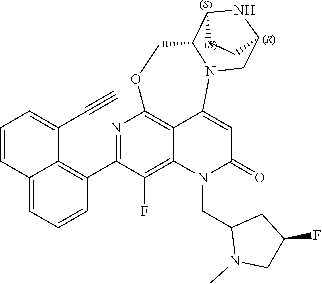 | (5aS,6S,9R)-2-(8- ethynylnaphthalen-1-yl)-1- fluoro-14-(((4R)-4-fluoro-1- methylpyrrolidin-2-yl)methyl)- 5a,6,7,8,9,10-hexahydro-5H-6,9- epiminoazepino[2′,1′:3,4][1,4] oxazepino[5,6,7- de][1,6]naphthyridin-13(14H)- one | 568 [M + H]⁺ |
| 286 | 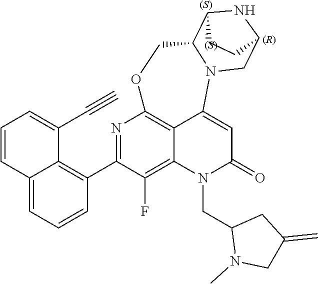 | (5aS,6S,9R)-2-(8- ethynylnaphthalen-1-yl)-1- fluoro-14-((1-methyl-4- methylenepyrrolidin-2- yl)methyl)-5a,6,7,8,9,10- hexahydro-5H-6,9- epiminoazepino[2′,1′:3,4][1,4] oxazepino[5,6,7- de][1,6]naphthyridin-13(14H)- one | 562 [M + H]⁺ |
| 287 | 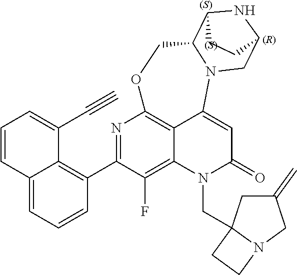 | (5aS,6S,9R)-2-(8- ethynylnaphthalen-1-yl)-1- fluoro-14-((3-methylene-1- azabicyclo[3.2.0]heptan-5- yl)methyl)-5a,6,7,8,9,10- hexahydro-5H-6,9- epiminoazepino[2′,1′:3,4][1,4] oxazepino[5,6,7- de][1,6]naphthyridin-13(14H)- one | 574 [M + H]⁺ |
| 288 | 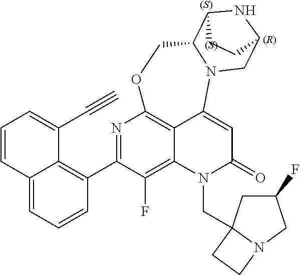 | (5aS,6S,9R)-2-(8- ethynylnaphthalen-1-yl)-1- fluoro-14-(((3R)-3-fluoro-1- azabicyclo[3.2.0]heptan-5- yl)methyl)-5a,6,7,8,9,10- hexahydro-5H-6,9- epiminoazepino[2′,1′:3,4][1,4] oxazepino[5,6,7- de][1,6]naphthyridin-13(14H)- one | 580 [M + H]⁺ |
| 289 | 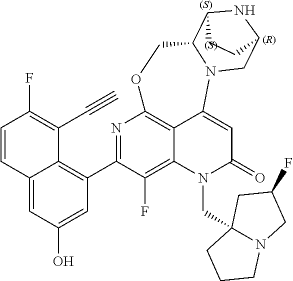 | (5aS,6S,9R)-2-(8-ethynyl-7- fluoro-3-hydroxynaphthalen-1 - yl)- 1 -fluoro-14-((2- methylenetetrahydro-1H- pyrrolizin-7a(5H)-yl)methyl)- 5a,6,7,8,9,10-hexahydro-5H-6,9- epiminoazepino[2′,1′:3,4][1,4] oxazepino[5,6,7- de][1,6]naphthyridin-13(14H)- one | 628 [M + H]⁺ |
| 290 | 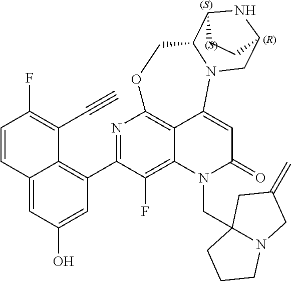 | (5aS,6S,9R)-14-((2,6- dimethylenetetrahydro-1H- pyrrolizin-7a(5H)-yl)methyl)-2- (8-ethynyl-7-fluoro-3- hydroxynaphthalen-1-yl)-1- fluoro-5a,6,7,8,9,10-hexahydro- 5H-6,9- epiminoazepino[2′,1′:3,4][1,4] oxazepino[5,6,7- de][1,6]naphthyridin-13(14H)- one | 622 [M + H]⁺ |
| 291 | 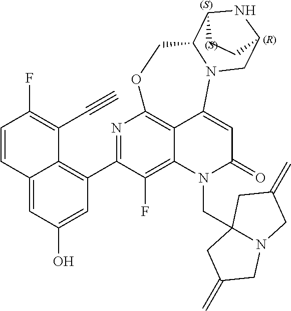 | (5aS,6S,9R)-2-(8-ethynyl-7- fluoro-3-hydroxynaphthalen-1- yl)-1-fluoro-14-(((2R,7aS)-2- fluorotetrahydro-1H-pyrrolizin- 7a(5H)-yl)methyl)-5a,6,7,8,9,10- hexahydro-5H-6,9- epiminoazepino[2′,1′:3,4][1,4] oxazepino[5,6,7- de][1,6]naphthyridin-13(14H)- one | 634 [M + H]⁺ |
| 292 | 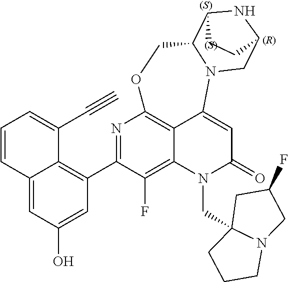 | (5aS,6S,9R)-2-(8-ethynyl-3- hydroxynaphthalen-1-yl)-1- fluoro-14-((2- methylenetetrahydro-1H- pyrrolizin-7a(5H)-yl)methyl)- 5a,6,7,8,9,10-hexahydro-5H-6,9- epiminoazepino[2′,1′:3,4][1,4] oxazepino[5,6,7- de][1,6]naphthyridin-13(14H)- one | 610 [M + H]⁺ |
| 293 | 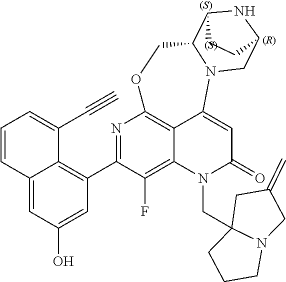 | (5aS,6S,9R)-2-(8-ethynyl-3- hydroxynaphthalen-1-yl)-1- fluoro-14-(((2R,7aS)-2- fluorotetrahydro-1H-pyrrolizin- 7a(5H)-yl)methyl)-5a,6,7,8,9,10- hexahydro-5H-6,9- epiminoazepino[2′,1′:3,4][1,4] oxazepino[5,6,7- de][1,6]naphthyridin-13(14H)- one | 604 [M + H]⁺ |
| 294 | 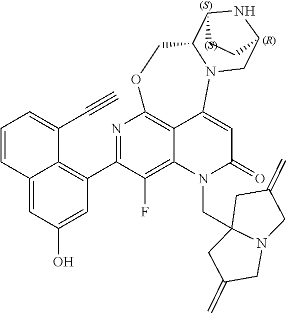 | (5aS,6S,9R)-14-((2,6- dimethylenetetrahydro-1H- pyrrolizin-7a(5H)-yl)methyl)-2- (8-ethynyl-3- hydroxynaphthalen-1-yl)-1- fluoro-5a,6,7,8,9,10-hexahydro- 5H-6,9- epiminoazepino[2′,1′:3,4][1,4] oxazepino[5,6,7- de][1,6]naphthyridin-13(14H)- one | 616 [M + H]⁺ |
| 295 | 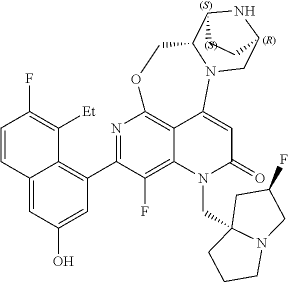 | (5aS,6S,9R)-2-(8-ethyl-7-fluoro- 3-hydroxynaphthalen-1-yl)-1- fluoro-14-((2- methylenetetrahydro-1H- pyrrolizin-7a(5H)-yl)methyl)- 5a,6,7,8,9,10-hexahydro-5H-6,9- epiminoazepino[2′,1′:3,4][1,4] oxazepino[5,6,7- de][1,6]naphthyridin-13(14H)- one | 632 [M + H]⁺ |
| 296 | 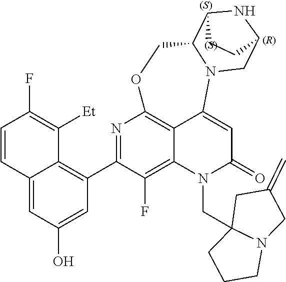 | (5aS,6S,9R)-2-(8-ethyl-7-fluoro- 3-hydroxynaphthalen-1-yl)-1- fluoro-14-(((2R,7aS)-2- fluorotetrahydro-1H-pyrrolizin- 7a(5H)-yl)methyl)-5a,6,7,8,9,10- hexahydro-5H-6,9- epiminoazepino[2′,1′:3,4][1,4] oxazepino[5,6,7- de][1,6]naphthyridin-13(14H)- one | 626 [M + H]⁺ |
| 297 | 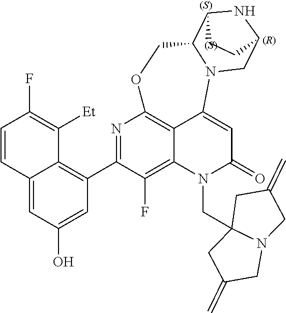 | (5aS,6S,9R)-14-((2,6- dimethylenetetrahydro-1H- pyrrolizin-7a(5H)-yl)methyl)-2- (8-ethyl-7-fluoro-3- hydroxynaphthalen-1-yl)-1- fluoro-5a,6,7,8,9,10-hexahydro- 5H-6,9- epiminoazepino[2′,1′:3,4][1,4] oxazepino[5,6,7- de][1,6]naphthyridin-13(14H)- one | 638 [M + H]⁺ |
| 298 | 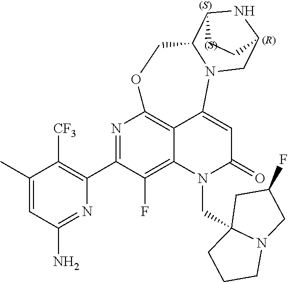 | (5aS,6S,9R)-2-(6-amino-4- methyl-3- (trifluoromethyl)pyridin-2-yl)-1- fluoro-14-((2- methylenetetrahydro-1H- pyrrolizin-7a(5H)-yl)methyl)- 5a,6,7,8,9,10-hexahydro-5H-6,9- epiminoazepino[2′,1′:3,4][1,4] oxazepino[5,6,7- de][1,6]naphthyridin-13(14H)- one | 618 [M + H]⁺ |
| 299 | 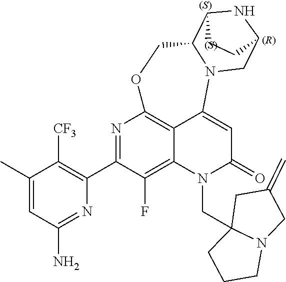 | (5aS,6S,9R)-2-(6-amino-4- methyl-3- (trifluoromethyl)pyridin-2-yl)- 14-((2,6-dimethylenetetrahydro- 1H-pyrrolizin-7a(5H)- yl)methyl)-1-fluoro- 5a,6,7,8,9,10-hexahydro-5H-6,9- epiminoazepino[2′,1′:3,4][1,4] oxazepino[5,6,7- de][1,6]naphthyridin-13(14H)- one | 612 [M + H]⁺ |
| 300 | 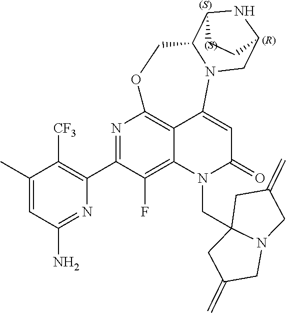 | (5aS,6S,9R)-2-(6-amino-4- methyl-3- (trifluoromethyl)pyridin-2-yl)-1- fluoro-14-(((2R,7aS)-2- fluorotetrahydro-1H-pyrrolizin- 7a(5H)-yl)methyl)-5a,6,7,8,9,10- hexahydro-5H-6,9- epiminoazepino[2′,1′:3,4][1,4] oxazepino[5,6,7- de][1,6]naphthyridin-13(14H)- one | 624 [M + H]⁺ |
| 301 | 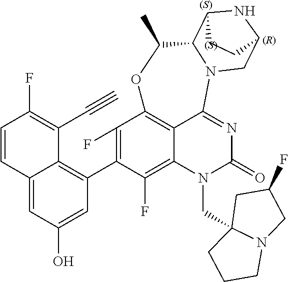 | (5S,5aS,6S,9R)-2-(8-ethynyl-7- fluoro-3-hydroxynaphthalen-1- yl)-1,3-difluoro-5-methyl-14- ((2-methylenetetrahydro-1H- pyrrolizin-7a(5H)-yl)methyl)- 5a,6,7,8,9,10-hexahydro-5H-6,9- epiminoazepino[2′,1′:3,4][1,4] oxazepino[5,6,7-de]quinazolin- 13(14H)-one | 660 [M + H]⁺ |
| 302 | 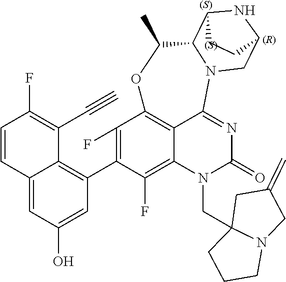 | (5S,5aS,6S,9R)-2-(8-ethyl-7- fluoro-3-hydroxynaphthalen-1- yl)-1,3-difluoro-14-(((2R,7aS)- 2-fluorotetrahydro-1H- pyrrolizin-7a(5H)-yl)methyl)-5- methyl-5a,6,7,8,9,10-hexahydro- 5H-6,9- epiminoazepino[2′,1′:3,4][1,4] oxazepino[5,6,7-de]quinazolin- 13(14H)-one | 654 [M + H]⁺ |
| 303 | 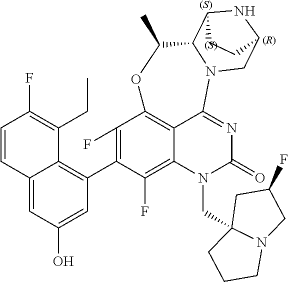 | (5S,5aS,6S,9R)-2-(8-ethyl-7- fluoro-3-hydroxynaphthalen-1- yl)-1,3-difluoro-5-methyl-14- ((2-methylenetetrahydro-1H- pyrrolizin-7a(5H)-yl)methyl)- 5a,6,7,8,9,10-hexahydro-5H-6,9- epiminoazepino[2′,1′:3,4][1,4] oxazepino[5,6,7-de]quinazolin- 13(14H)-one | 664 [M + H]⁺ |
| 304 | 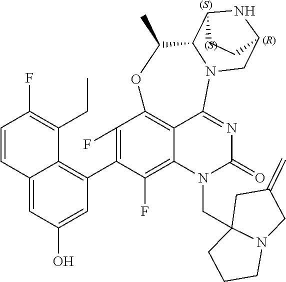 | (5S,5aS,6S,9R)-2-(8-ethynyl-7- fluoro-3-hydroxynaphthalen-1- yl)-1,3-difluoro-14-(((2R,7aS)- 2-fluorotetrahydro-1H- pyrrolizin-7a(5H)-yl)methyl)-5- methyl-5a,6,7,8,9,10-hexahydro- 5H-6,9- epiminoazepino[2′,1′:3,4][1,4] oxazepino[5,6,7-de]quinazolin- 13(14H)-one | 658 [M + H]⁺ |
| 305 | 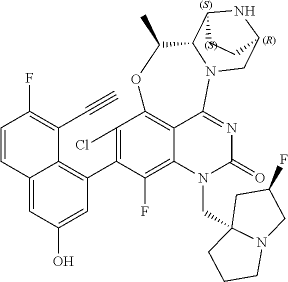 | (5S,5aS,6S,9R)-3-chloro-2-(8- ethynyl-7-fluoro-3- hydroxynaphthalen-1-yl)-1- fluoro-5-methyl-14-((2- methylenetetrahydro-1H- pyrrolizin-7a(5H)-yl)methyl)- 5a,6,7,8,9,10-hexahydro-5H-6,9- epiminoazepino[2′,1′:3,4][1,4] oxazepino[5,6,7-de]quinazolin- 13(14H)-one | 676 [M + H]⁺ |
| 306 | 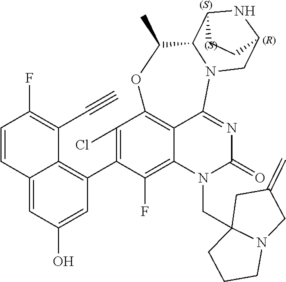 | (5S,5aS,6S,9R)-3-chloro-2-(8- ethyl-7-fluoro-3- hydroxynaphthalen-1-yl)-1- fluoro-14-(((2R,7aS)-2- fluorotetrahydro-1H-pyrrolizin- 7a(5H)-yl)methyl)-5-methyl- 5a,6,7,8,9,10-hexahydro-5H-6,9- epiminoazepino[2′,1′:3,4][1,4] oxazepino[5,6,7-de]quinazolin- 13(14H)-one | 670 [M + H]⁺ |
| 307 | 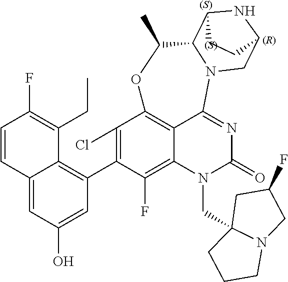 | (5S,5aS,6S,9R)-3-chloro-2-(8- ethyl-7-fluoro-3- hydroxynaphthalen-1-yl)-1- fluoro-5-methyl-14-((2- methylenetetrahydro-1H- pyrrolizin-7a(5H)-yl)methyl)- 5a,6,7,8,9,10-hexahydro-5H-6,9- epiminoazepino[2′,1′:3,4][1,4] oxazepino[5,6,7-de]quinazolin- 13(14H)-one | 680 [M + H]⁺ |
| 308 | 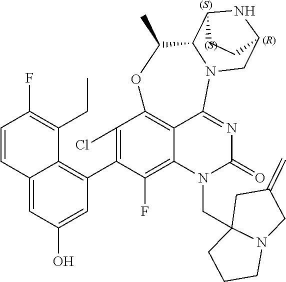 | (5S,5aS,6S,9R)-3-chloro-2-(8- ethynyl-7-fluoro-3- hydroxynaphthalen-1-yl)-1- fluoro-14-(((2R,7aS)-2- fluorotetrahydro-1H-pyrrolizin- 7a(5H)-yl)methyl)-5-methyl- 5a,6,7,8,9,10-hexahydro-5H-6,9- epiminoazepino[2′,1′:3,4][1,4] oxazepino[5,6,7-de]quinazolin- 13(14H)-one | 674 [M + H]⁺ |
| 309 | 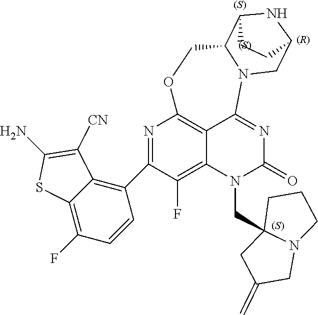 | 2-amino-7-fluoro-4- ((5aS,6S,9R)-1-fluoro-13-(((S)- 2-methylenetetrahydro-1H- pyrrolizin-7a(5H)-yl)methyl)- 12-oxo-5a,6,7,8,9,10,12,13- octahydro-5H-4-oxa- 3,10a,11,13,14-pentaaza-6,9- methanonaphtho[1,8- ab]heptalen-2- yl)benzo[b]thiophene-3- carbonitrile | 628 [M + H]⁺ |
| 311 | 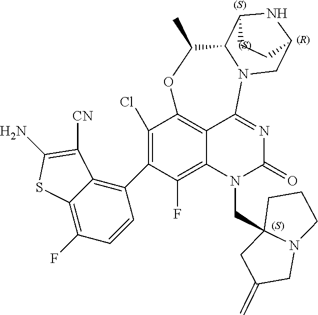 | 2-amino-4-((5S,5aS,6S,9R)-3- chloro-1-fluoro-5-methyl-14- (((S)-2-methylenetetrahydro-1H- pyrrolizin-7a(5H)-yl)methyl)- 13-oxo-5a,6,7,8,9,10,13,14- octahydro-5H-6,9- epiminoazepino[2′,1′:3,4][1,4] oxazepino[5,6,7-de]quinazolin-2- yl)-7-fluorobenzo[b]thiophene- 3-carbonitrile | 676 [M + H]⁺ |
| 312 | 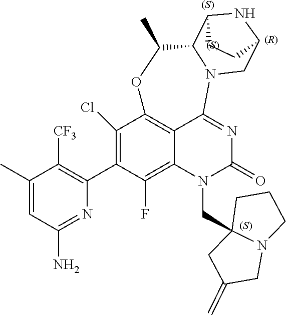 | (5S,5aS,6S,9R)-2-(6-amino-4- methyl-3- (trifluoromethyl)pyridin-2-yl)-3- chloro-1-fluoro-5-methyl-14- (((S)-2-methylenetetrahydro-1H- pyrrolizin-7a(5H)-yl)methyl)- 5a,6,7,8,9,10-hexahydro-5H-6,9- epiminoazepino[2′,1′:3,4][1,4] oxazepino[5,6,7-de]quinazolin- 13(14H)-one | 660 [M + H]⁺ |
| 313 | 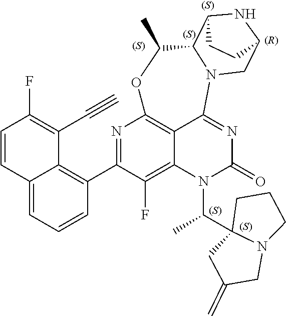 | (5S,5aS,6S,9R)-2-(8-ethynyl-7- fluoronaphthalen-1-yl)-1-fluoro- 5-methyl-13-((S)-1-((S)-2- methylenetetrahydro-1H- pyrrolizin-7a(5H)-yl)ethyl)- 5a,6,7,8,9,10-hexahydro-5H-4- oxa-3,10a,11,13,14-pentaaza- 6,9-methanonaphtho[1,8- ab]heptalen-12(13H)-one | 634 [M + H]⁺ |
| 314 | 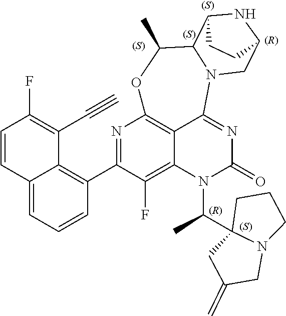 | (5S,5aS,6S,9R)-2-(8-ethynyl-7- fluoronaphthalen-1-yl)-1-fluoro- 5-methyl-13-((R)-1-((S)-2- methylenetetrahydro-1H- pyrrolizin-7a(5H)-yl)ethyl)- 5a,6,7,8,9,10-hexahydro-5H-4- oxa-3,10a,11,13,14-pentaaza- 6,9-methanonaphtho[1,8- ab]heptalen-12(13H)-one | 634 [M + H]⁺ |
| 315 | 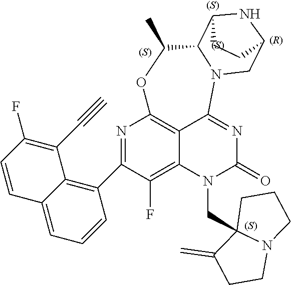 | (5S,5aS,6S,9R)-2-(8-ethynyl-7- fluoronaphthalen-1-yl)-1-fluoro- 5-methyl-13-(((S)-1- methylenetetrahydro-1H- pyrrolizin-7a(5H)-yl)methyl)- 5a,6,7,8,9,10-hexahydro-5H-4- oxa-3,10a,11,13,14-pentaaza- 6,9-methanonaphtho[1,8- ab]heptalen-12(13H)-one | 620 [M + H]⁺ |
| 316 | 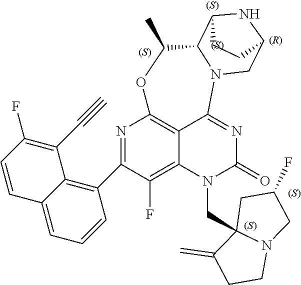 | (5S,5aS,6S,9R)-2-(8-ethynyl-7- fluoronaphthalen-1-yl)-1-fluoro- 13-(((6S,7aS)-6-fluoro-1- methylenetetrahydro-1H- pyrrolizin-7a(5H)-yl)methyl)-5- methyl-5a,6,7,8,9,10-hexahydro- 5H-4-oxa-3,10a,11,13,14- pentaaza-6,9- methanonaphtho[1,8- ab]heptalen-12(13H)-one | 638 [M + H]⁺ |
| 317 | 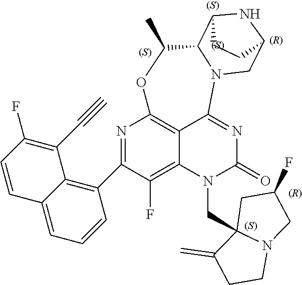 | (5S,5aS,6S,9R)-2-(8-ethynyl-7- fluoronaphthalen-1-yl)-1-fluoro- 13-(((6R,7aS)-6-fluoro-1- methylenetetrahydro-1H- pyrrolizin-7a(5H)-yl)methyl)-5- methyl-5a,6,7,8,9,10-hexahydro- 5H-4-oxa-3,10a,11,13,14- pentaaza-6,9- methanonaphtho[1,8- ab]heptalen-12(13H)-one | 638 [M + H]⁺ |
| 318 | 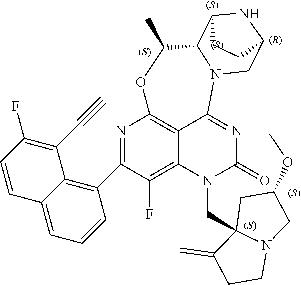 | (5S,5aS,6S,9R)-2-(8-ethynyl-7- fluoronaphthalen-1-yl)-1-fluoro- 13-(((6S,7aS)-6-methoxy-1- methylenetetrahydro-1H- pyrrolizin-7a(5H)-yl)methyl)-5- methyl-5a,6,7,8,9,10-hexahydro- 5H-4-oxa-3,10a,11,13,14- pentaaza-6,9- methanonaphtho[1,8- ab]heptalen-12(13H)-one | 650 [M + H]⁺ |
| 319 | 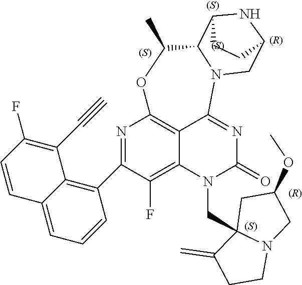 | (5S,5aS,6S,9R)-2-(8-ethynyl-7- fluoronaphthalen-1-yl)-1-fluoro- 13-(((6R,7aS)-6-methoxy-1- methylenetetrahydro-1H- pyrrolizin-7a(5H)-yl)methyl)-5- methyl-5a,6,7,8,9,10-hexahydro- 5H-4-oxa-3,10a,11,13,14- pentaaza-6,9- methanonaphtho[1,8- ab]heptalen-12(13H)-one | 650 [M + H]⁺ |
| 320 | 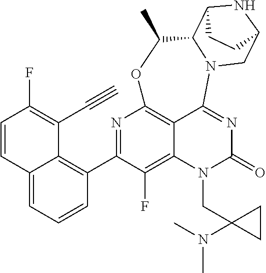 [SMILES: C#Cc1c(F)ccc2cccc(-c3nc4c5c(nc(=O)n(CC6(N(C)C)CC6)c5c3F)N3C[C@H]5CC[C@H](N5)[C@H]3[C@H](C)O4)c12] | (5S,5aS,6S,9R)-13-((1- (dimethylamino)cyclopropyl) methyl)-2-(8-ethynyl-7- fluoronaphthalen-1-yl)-1-fluoro- 5-methyl-5a,6,7,8,9,10- hexahydro-5H-4-oxa- 3,10a,11,13,14-pentaaza-6,9- methanonaphtho[1,8- ab]heptalen-12(13H)-one | 582 [M + H]⁺ |
| 321 | 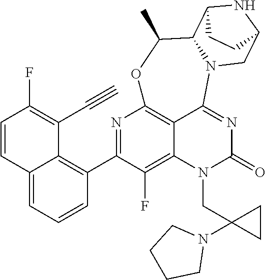 [SMILES: C#Cc1c(F)ccc2cccc(-c3nc4c5c(nc(=O)n(CC6(N7CCCC7)CC6)c5c3F)N3C[C@H]5CC[C@H](N5)[C@H]3[C@H](C)O4)c12] | (5S,5aS,6S,9R)-2-(8-ethynyl-7- fluoronaphthalen-1-yl)-1-fluoro- 5-methyl-13-((1-(pyrrolidin-1- yl)cyclopropyl)methyl)- 5a,6,7,8,9,10-hexahydro-5H-4- oxa-3,10a,11,13,14-pentaaza- 6,9-methanonaphtho[1,8- ab]heptalen-12(13H)-one | 608 [M + H]⁺ |
| 322 | 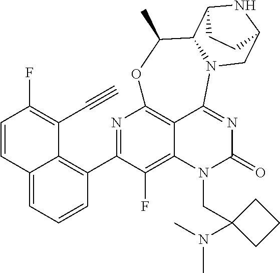 [SMILES: C#Cc1c(F)ccc2cccc(-c3nc4c5c(nc(=O)n(CC6(N(C)C)CCC6)c5c3F)N3C[C@H]5CC[C@H](N5)[C@H]3[C@H](C)O4)c12] | (5S,5aS,6S,9R)-13-((1- (dimethylamino)cyclobutyl) methyl)-2-(8-ethynyl-7- fluoronaphthalen-1-yl)-1-fluoro- 5-methyl-5a,6,7,8,9,10- hexahydro-5H-4-oxa- 3,10a,11,13,14-pentaaza-6,9- methanonaphtho[1,8- ab]heptalen-12(13H)-one | 596 [M + H]⁺ |
| 323 | 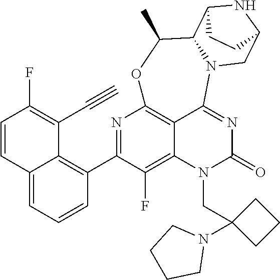 [SMILES: C#Cc1c(F)ccc2cccc(-c3nc4c5c(nc(=O)n(CC6(N7CCCC7)CCC6)c5c3F)N3C[C@H]5CC[C@H](N5)[C@H]3[C@H](C)O4)c12] | (5S,5aS,6S,9R)-2-(8-ethynyl-7- fluoronaphthalen-1-yl)-1-fluoro- 5-methyl-13-((1-(pyrrolidin-1- yl)cyclobutyl)methyl)- 5a,6,7,8,9,10-hexahydro-5H-4- oxa-3,10a,11,13,14-pentaaza- 6,9-methanonaphtho[1,8- ab]heptalen-12(13H)-one | 622 [M + H]⁺ |
| 324a | 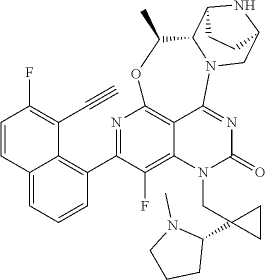 [SMILES: C#Cc1c(F)ccc2cccc(-c3nc4c5c(nc(=O)n(CC6([C@@H]7CCCN7C)CC6)c5c3F)N3C[C@H]5CC[C@H](N5)[C@H]3[C@H](C)O4)c12] | (5S,5aS,6S,9R)-2-(8-ethynyl-7- fluoronaphthalen-1-yl)-1-fluoro- 5-methyl-13-((1-((S)-1- methylpyrrolidin-2- yl)cyclopropyl)methyl)- 5a,6,7,8,9,10-hexahydro-5H-4- oxa-3,10a,11,13,14-pentaaza- 6,9-methanonaphtho[1,8- ab]heptalen-12(13H)-one Isomer 1 (the first eluting isomer) | 622 [M + H]⁺ |
| 324b | 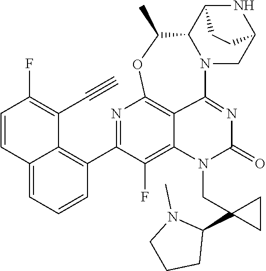 [SMILES: C#Cc1c(F)ccc2cccc(-c3nc4c5c(nc(=O)n(CC6([C@H]7CCCN7C)CC6)c5c3F)N3C[C@H]5CC[C@H](N5)[C@H]3[C@H](C)O4)c12] | (5S,5aS,6S,9R)-2-(8-ethynyl-7- fluoronaphthalen-1-yl)-1-fluoro- 5-methyl-13-((1-((R)-1- methylpyrrolidin-2- yl)cyclopropyl)methyl)- 5a,6,7,8,9,10-hexahydro-5H-4- oxa-3,10a,11,13,14-pentaaza- 6,9-methanonaphtho[1,8- ab]heptalen-12(13H)-one Isomer 2 (the second eluting isomer) | 622 [M + H]⁺ |
| 325a | 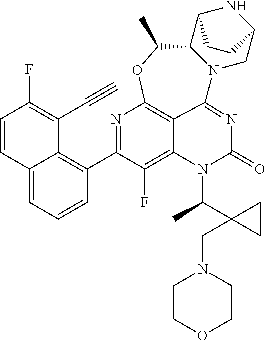 [SMILES: C#Cc1c(F)ccc2cccc(-c3nc4c5c(nc(=O)n([C@H](C)C6(CN7CCOCC7)CC6)c5c3F)N3C[C@H]5CC[C@H](N5)[C@H]3[C@H](C)O4)c12] | (5S,5aS,6S,9R)-2-(8-ethynyl-7- fluoronaphthalen-1-yl)-1-fluoro- 5-methyl-13-((R)-1-(1- (morpholinomethyl)cyclopropyl) ethyl)-5a,6,7,8,9,10-hexahydro- 5H-4-oxa-3,10a,11,13,14- pentaaza-6,9- methanonaphtho[1,8- ab]heptalen-12(13H)-one Isomer 1 (the first eluting isomer) | 652 [M + H]⁺ |
| 325b | 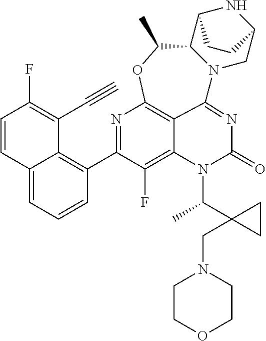 [SMILES: C#Cc1c(F)ccc2cccc(-c3nc4c5c(nc(=O)n([C@@H](C)C6(CN7CCOCC7)CC6)c5c3F)N3C[C@H]5CC[C@H](N5)[C@H]3[C@H](C)O4)c12] | (5S,5aS,6S,9R)-2-(8-ethynyl-7- fluoronaphthalen-1-yl)-1-fluoro- 5-methyl-13-((S)-1-(1- (morpholinomethyl)cyclopropyl) ethyl)-5a,6,7,8,9,10-hexahydro- 5H-4-oxa-3,10a,11,13,14- pentaaza-6,9- methanonaphtho[1,8- ab]heptalen-12(13H)-one Isomer 2 (the second eluting isomer) | 652 [M + H]⁺ |
| 326a | 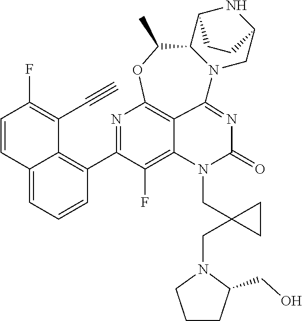 [SMILES: C#Cc1c(F)ccc2cccc(-c3nc4c5c(nc(=O)n(CC6(CN7CCC[C@H]7CO)CC6)c5c3F)N3C[C@H]5CC[C@H](N5)[C@H]3[C@H](C)O4)c12] | (5S,5aS,6S,9R)-2-(8-ethynyl-7- fluoronaphthalen-1-yl)-1-fluoro- 13-((1-(((S)-2- (hydroxymethyl)pyrrolidin-1- yl)methyl)cyclopropyl)methyl)- 5-methyl-5a,6,7,8,9,10- hexahydro-5H-4-oxa- 3,10a,11,13,14-pentaaza-6,9- methanonaphtho[1,8- ab]heptalen-12(13H)-one Isomer 1 (the first eluting isomer) | 652 [M + H]⁺ |
| 326b | 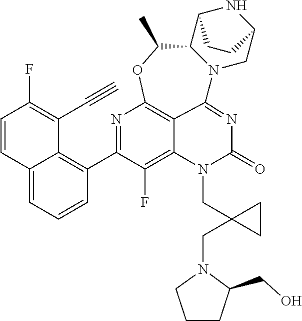 [SMILES: C#Cc1c(F)ccc2cccc(-c3nc4c5c(nc(=O)n(CC6(CN7CCC[C@@H]7CO)CC6)c5c3F)N3C[C@H]5CC[C@H](N5)[C@H]3[C@H](C)O4)c12] | (5S,5aS,6S,9R)-2-(8-ethynyl-7- fluoronaphthalen-1-yl)-1-fluoro- 13-((1-(((R)-2- (hydroxymethyl)pyrrolidin-1- yl)methyl)cyclopropyl)methyl)- 5-methyl-5a,6,7,8,9,10- hexahydro-5H-4-oxa- 3,10a,11,13,14-pentaaza-6,9- methanonaphtho[1,8- ab]heptalen-12(13H)-one Isomer 2 (the second eluting isomer) | 652 [M + H]⁺ |
| 327a | 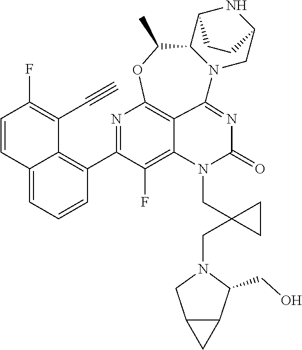 [SMILES: C#Cc1c(F)ccc2cccc(-c3nc4c5c(nc(=O)n(CC6(CN7CC8CC8[C@H]7CO)CC6)c5c3F)N3C[C@H]5CC[C@H](N5)[C@H]3[C@H](C)O4)c12] | (5S,5aS,6S,9R)-2-(8-ethynyl-7- fluoronaphthalen-1-yl)-1-fluoro- 13-((1-(((2S)-2- (hydroxymethyl)-3- azabicyclo[3.1.0]hexan-3- yl)methyl)cyclopropyl)methyl)- 5-methyl-5a,6,7,8,9,10- hexahydro-5H-4-oxa- 3,10a,11,13,14-pentaaza-6,9- methanonaphtho[1,8- ab]heptalen-12(13H)-one Isomer 1 (the first eluting isomer) | 664 [M + H]⁺ |
| 327b | 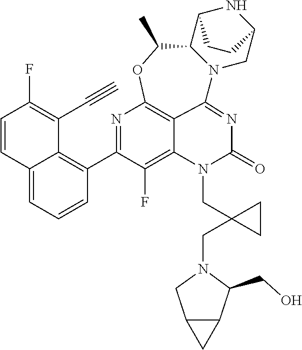 [SMILES: C#Cc1c(F)ccc2cccc(-c3nc4c5c(nc(=O)n(CC6(CN7CC8CC8[C@@H]7CO)CC6)c5c3F)N3C[C@H]5CC[C@H](N5)[C@H]3[C@H](C)O4)c12] | (5S,5aS,6S,9R)-2-(8-ethynyl-7- fluoronaphthalen-1-yl)-1-fluoro- 13-((1-(((2R)-2- (hydroxymethyl)-3- azabicyclo[3.1.0]hexan-3- yl)methyl)cyclopropyl)methyl)- 5-methyl-5a,6,7,8,9,10- hexahydro-5H-4-oxa- 3,10a,11,13,14-pentaaza-6,9- methanonaphtho[1,8- ab]heptalen-12(13H)-one Isomer 2 (the second eluting isomer) | 664 [M + H]⁺ |
| 328 | 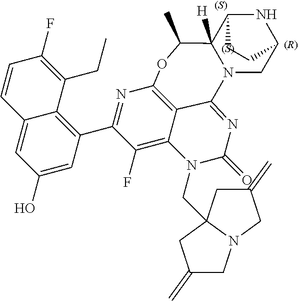 | (5S,5aS,6S,9R)-14-((2,6- dimethylenetetrahydro-1H- pyrrolizin-7a(5H)-yl)methyl)-2- (8-ethyl-7-fluoro-3- hydroxynaphthalen-1-yl)-1- fluoro-5-methyl-5a,6,7,8,9,10- hexahydro-5H-6,9- epiminoazepino[2′,1′:3,4][1,4] oxazepino[5,6,7- de][1,6]naphthyridin-13(14H)- one | 651 [M + H]⁺ |
| 329 | 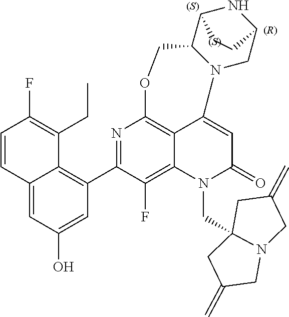 | (5aS,6S,9R)-14-((2,6- dimethylenetetrahydro-1H- pyrrolizin-7a(5H)-yl)methyl)-2- (8-ethyl-7-fluoro-3- hydroxynaphthalen-1-yl)-1- fluoro-5a,6,7,8,9,10-hexahydro- 5H-6,9- epiminoazepino[2′,1′:3,4][1,4] oxazepino[5,6,7- de][1,6]naphthyridin-13(14H)- one | 637 [M + H]⁺ |
| 330 | 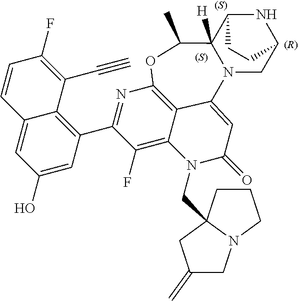 | (5S,5aS,6S,9R)-2-(8-ethynyl-7- fluoro-3-hydroxynaphthalen-1- yl)-1-fluoro-5-methyl-14-(((S)- 2-methylenetetrahydro-1H- pyrrolizin-7a(5H)-yl)methyl)- 5a,6,7,8,9,10-hexahydro-5H-6,9- epiminoazepino[2′,1′:3,4][1,4] oxazepino[5,6,7- de][1,6]naphthyridin-13(14H)- one | 635 [M + H]⁺ |
| 331 | 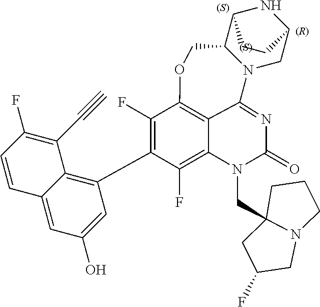 | (5aS,6S,9R)-2-(8-ethynyl-7- fluoro-3-hydroxynaphthalen-1- yl)-1,3-difluoro-14-(((2R,7aS)- 2-fluorotetrahydro-1H- pyrrolizin-7a(5H)-yl)methyl)- 5a,6,7,8,9,10-hexahydro-5H-6,9- epiminoazepino[2′,1′:3,4][1,4] oxazepino[5,6,7-de]quinolin- 13(14H)-one | 644 [M + H]⁺ |
| 332 | 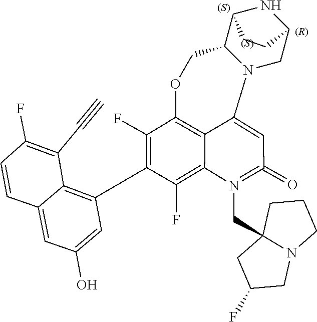 | (5aS,6S,9R)-2-(8-ethynyl-7- fluoro-3-hydroxynaphthalen-1- yl)-1,3-difluoro-14-(((2R,7aS)- 2-fluorotetrahydro-1H- pyrrolizin-7a(5H)-yl)methyl)- 5a,6,7,8,9,10-hexahydro-5H-6,9- epiminoazepino[2′,1′:3,4][1,4] oxazepino[5,6,7-de]quinolin- 13(14H)-one | 644 [M + H]⁺ |
| 333 | 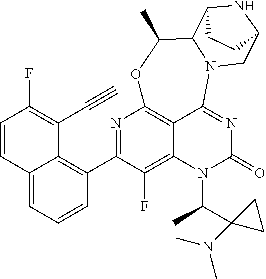 [SMILES: C#Cc1c(F)ccc2cccc(-c3nc4c5c(nc(=O)n([C@H](C)C6(N(C)C)CC6)c5c3F)N3C[C@H]5CC[C@H](N5)C3[C@H](C)O4)c12] | (5S,5aS,6S,9R)-13-((R)-1-(1- (dimethylamino)cyclopropyl) ethyl)-2-(8-ethynyl-7- fluoronaphthalen-1-yl)-1-fluoro- 5-methyl-5a,6,7,8,9,10- hexahydro-5H-4-oxa- 3,10a,11,13,14-pentaaza-6,9- methanonaphtho[1,8- ab]heptalen-12(13H)-one | 596 [M + H]⁺ |
| 334 | 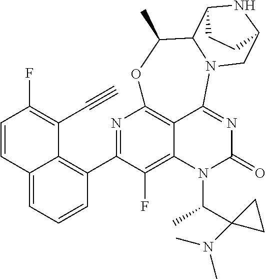 [SMILES: C#Cc1c(F)ccc2cccc(-c3nc4c5c(nc(=O)n([C@@H](C)C6(N(C)C)CC6)c5c3F)N3C[C@H]5CC[C@H](N5)C3[C@H](C)O4)c12] | (5S,5aS,6S,9R)-13-((S)-1-(1- (dimethylamino)cyclopropyl) ethyl)-2-(8-ethynyl-7- fluoronaphthalen-1-yl)-1-fluoro- 5-methyl-5a,6,7,8,9,10- hexahydro-5H-4-oxa- 3,10a,11,13,14-pentaaza-6,9- methanonaphtho[1,8- ab]heptalen-12(13H)-one | 596 [M + H]⁺ |
| 335 | 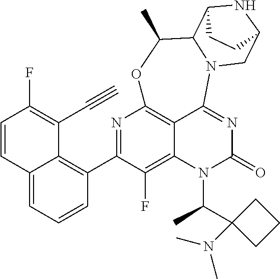 [SMILES: C#Cc1c(F)ccc2cccc(-c3nc4c5c(nc(=O)n([C@H](C)C6(N(C)C)CCC6)c5c3F)N3C[C@H]5CC[C@H](N5)C3[C@H](C)O4)c12] | (5S,5aS,6S,9R)-13-((R)-1-(1- (dimethylamino)cyclobutyl) ethyl)-2-(8-ethynyl-7- fluoronaphthalen-1-yl)-1-fluoro- 5-methyl-5a,6,7,8,9,10- hexahydro-5H-4-oxa- 3,10a,11,13,14-pentaaza-6,9- methanonaphtho[1,8- ab]heptalen-12(13H)-one | 610 [M + H]⁺ |
| 336 | 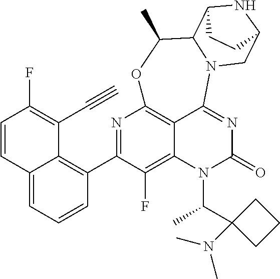 [SMILES: C#Cc1c(F)ccc2cccc(-c3nc4c5c(nc(=O)n([C@@H](C)C6(N(C)C)CCC6)c5c3F)N3C[C@H]5CC[C@H](N5)C3[C@H](C)O4)c12] | (5S,5aS,6S,9R)-13-((S)-1-(1- (dimethylamino)cyclobutyl) ethyl)-2-(8-ethynyl-7- fluoronaphthalen-1-yl)-1-fluoro- 5-methyl-5a,6,7,8,9,10- hexahydro-5H-4-oxa- 3,10a,11,13,14-pentaaza-6,9- methanonaphtho[1,8- ab]heptalen-12(13H)-one | 610 [M + H]⁺ |
| 337 | 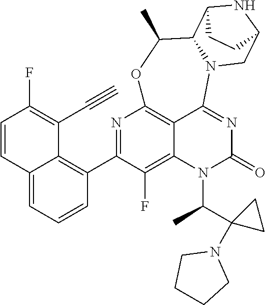 [SMILES: C#Cc1c(F)ccc2cccc(-c3nc4c5c(nc(=O)n([C@H](C)C6(N7CCCC7)CC6)c5c3F)N3C[C@H]5CC[C@H](N5)[C@H]3[C@H](C)O4)c12] | (5S,5aS,6S,9R)-2-(8-ethynyl-7- fluoronaphthalen-1-yl)-1-fluoro- 5-methyl-13-((R)-1-(1- (pyrrolidin-1- yl)cyclopropyl)ethyl)- 5a,6,7,8,9,10-hexahydro-5H-4- oxa-3,10a,11,13,14-pentaaza- 6,9-methanonaphtho[1,8- ab]heptalen-12(13H)-one | 622 [M + H]⁺ |
| 338 | 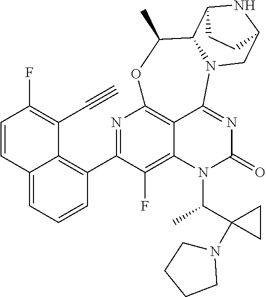 [SMILES: C#Cc1c(F)ccc2cccc(-c3nc4c5c(nc(=O)n([C@@H](C)C6(N7CCCC7)CC6)c5c3F)N3C[C@H]5CC[C@H](N5)[C@H]3[C@H](C)O4)c12] | (5S,5aS,6S,9R)-2-(8-ethynyl-7- fluoronaphthalen-1-yl)-1-fluoro- 5-methyl-13-((S)-1-(1- (pyrrolidin-1- yl)cyclopropyl)ethyl)- 5a,6,7,8,9,10-hexahydro-5H-4- oxa-3,10a,11,13,14-pentaaza- 6,9-methanonaphtho[1,8- ab]heptalen-12(13H)-one | 622 [M + H]⁺ |
| 339 | 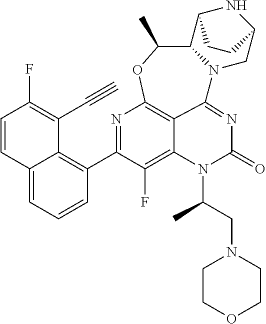 [SMILES: C#Cc1c(F)ccc2cccc(-c3nc4c5c(nc(=O)n([C@H](C)CN6CCOCC6)c5c3F)N3C[C@H]5CC[C@H](N5)[C@H]3[C@H](C)O4)c12] | (5S,5aS,6S,9R)-2-(8-ethynyl-7- fluoronaphthalen-1-yl)-1-fluoro- 5-methyl-13-((R)-1- morpholinopropan-2-yl)- 5a,6,7,8,9,10-hexahydro-5H-4- oxa-3,10a,11,13,14-pentaaza- 6,9-methanonaphtho[1,8- ab]heptalen-12(13H)-one | 612 [M + H]⁺ |
| 340 | 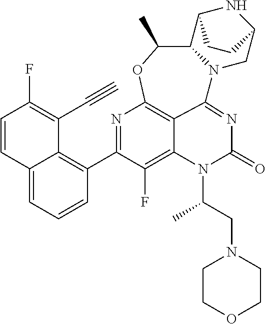 [SMILES: C#Cc1c(F)ccc2cccc(-c3nc4c5c(nc(=O)n([C@@H](C)CN6CCOCC6)c5c3F)N3C[C@H]5CC[C@H](N5)[C@H]3[C@H](C)O4)c12] | (5S,5aS,6S,9R)-2-(8-ethynyl-7- fluoronaphthalen-1-yl)-1-fluoro- 5-methyl-13-((S)-1- morpholinopropan-2-yl)- 5a,6,7,8,9,10-hexahydro-5H-4- oxa-3,10a,11,13,14-pentaaza- 6,9-methanonaphtho[1,8- ab]heptalen-12(13H)-one | 612 [M + H]⁺ |
| 341 | 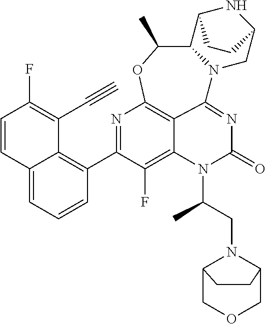 [SMILES: C#Cc1c(F)ccc2cccc(-c3nc4c5c(nc(=O)n([C@H](C)CN6C7CCC6COC7)c5c3F)N3C[C@H]5CC[C@H](N5)[C@H]3[C@H](C)O4)c12] | (5S,5aS,6S,9R)-13-((R)-1- ((1R,5S)-3-oxa-8- azabicyclo[3.2.1]octan-8- yl)propan-2-yl)-2-(8-ethynyl-7- fluoronaphthalen-1-yl)-1-fluoro- 5-methyl-5a,6,7,8,9,10- hexahydro-5H-4-oxa- 3,10a,11,13,14-pentaaza-6,9- methanonaphtho[1,8- ab]heptalen-12(13H)-one | 638 [M + H]⁺ |
| 342 | 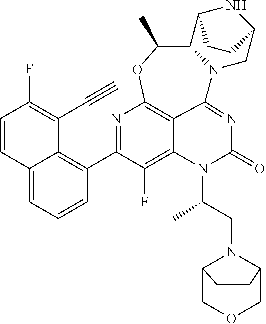 [SMILES: C#Cc1c(F)ccc2cccc(-c3nc4c5c(nc(=O)n([C@@H](C)CN6C7CCC6COC7)c5c3F)N3C[C@H]5CC[C@H](N5)[C@H]3[C@H](C)O4)c12] | (5S,5aS,6S,9R)-13-((S)-1- ((1R,5S)-3-oxa-8- azabicyclo[3.2.1]octan-8- yl)propan-2-yl)-2-(8-ethynyl-7- fluoronaphthalen-1-yl)-1-fluoro- 5-methyl-5a,6,7,8,9,10- hexahydro-5H-4-oxa- 3,10a,11,13,14-pentaaza-6,9- methanonaphtho[1,8- ab]heptalen-12(13H)-one | 638 [M + H]⁺ |
| 343 | 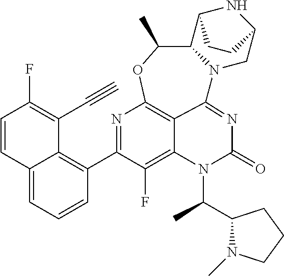 [SMILES: C#Cc1c(F)ccc2cccc(-c3nc4c5c(nc(=O)n([C@H](C)[C@@H]6CCCN6C)c5c3F)N3C[C@H]5CC[C@H](N5)[C@H]3[C@H](C)O4)c12] | (5S,5aS,6S,9R)-2-(8-ethynyl-7- fluoronaphthalen-1-yl)-1-fluoro- 5-methyl-13-((R)-1-((S)-1- methylpyrrolidin-2-yl)ethyl)- 5a,6,7,8,9,10-hexahydro-5H-4- oxa-3,10a,11,13,14-pentaaza- 6,9-methanonaphtho[1,8- ab]heptalen-12(13H)-one | 596 [M + H]⁺ |
| 344 | 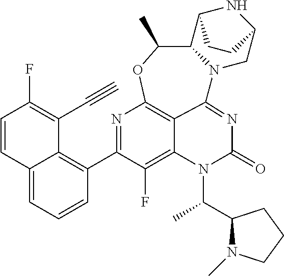 [SMILES: C#Cc1c(F)ccc2cccc(-c3nc4c5c(nc(=O)n([C@@H](C)[C@H]6CCCN6C)c5c3F)N3C[C@H]5CC[C@H](N5)[C@H]3[C@H](C)O4)c12] | (5S,5aS,6S,9R)-2-(8-ethynyl-7- fluoronaphthalen-1-yl)-1-fluoro- 5-methyl-13-((S)-1-((R)-1- methylpyrrolidin-2-yl)ethyl)- 5a,6,7,8,9,10-hexahydro-5H-4- oxa-3,10a,11,13,14-pentaaza- 6,9-methanonaphtho[1,8- ab]heptalen-12(13H)-one | 596 [M + H]⁺ |
| 345 | 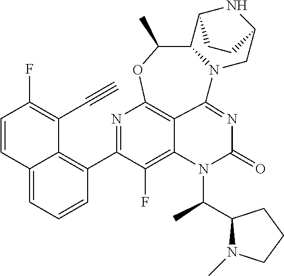 [SMILES: C#Cc1c(F)ccc2cccc(-c3nc4c5c(nc(=O)n([C@H](C)[C@H]6CCCN6C)c5c3F)N3C[C@H]5CC[C@H](N5)[C@H]3[C@H](C)O4)c12] | (5S,5aS,6S,9R)-2-(8-ethynyl-7- fluoronaphthalen-1-yl)-1-fluoro- 5-methyl-13-((R)-1-((R)-1- methylpyrrolidin-2-yl)ethyl)- 5a,6,7,8,9,10-hexahydro-5H-4- oxa-3,10a,11,13,14-pentaaza- 6,9-methanonaphtho[1,8- ab]heptalen-12(13H)-one | 596 [M + H]⁺ |
| 346 | 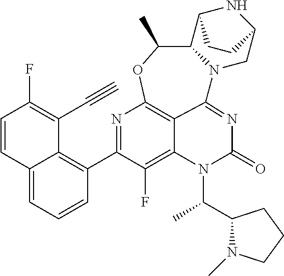 [SMILES: C#Cc1c(F)ccc2cccc(-c3nc4c5c(nc(=O)n([C@@H](C)[C@@H]6CCCN6C)c5c3F)N3C[C@H]5CC[C@H](N5)[C@H]3[C@H](C)O4)c12] | (5S,5aS,6S,9R)-2-(8-ethynyl-7- fluoronaphthalen-1-yl)-1-fluoro- 5-methyl-13-((S)-1-((S)-1- methylpyrrolidin-2-yl)ethyl)- 5a,6,7,8,9,10-hexahydro-5H-4- oxa-3,10a,11,13,14-pentaaza- 6,9-methanonaphtho[1,8- ab]heptalen-12(13H)-one | 596 [M + H]⁺ |
| 347 | 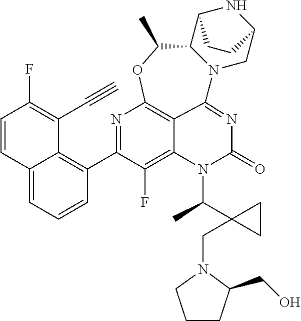 [SMILES: C#Cc1c(F)ccc2cccc(-c3nc4c5c(nc(=O)n([C@H](C)C6(CN7CCC[C@@H]7CO)CC6)c5c3F)N3C[C@H]5CC[C@H](N5)[C@H]3[C@H](C)O4)c12] | (5S,5aS,6S,9R)-2-(8-ethynyl-7- fluoronaphthalen-1-yl)-1-fluoro- 13-((S)-1-(1-(((R)-2- (hydroxymethyl)pyrrolidin-1- yl)methyl)cyclopropyl)ethyl)-5- methyl-5a,6,7,8,9,10-hexahydro- 5H-4-oxa-3,10a,11,13,14- pentaaza-6,9- methanonaphtho[1,8- ab]heptalen-12(13H)-one | 666 [M + H]⁺ |
| 348 | 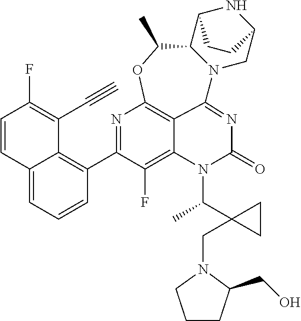 [SMILES: C#Cc1c(F)ccc2cccc(-c3nc4c5c(nc(=O)n([C@@H](C)C6(CN7CCC[C@@H]7CO)CC6)c5c3F)N3C[C@H]5CC[C@H](N5)[C@H]3[C@H](C)O4)c12] | (5S,5aS,6S,9R)-2-(8-ethynyl-7- fluoronaphthalen-1-yl)-1-fluoro- 13-((R)-1-(1-(((R)-2- (hydroxymethyl)pyrrolidin-1- yl)methyl)cyclopropyl)ethyl)-5- methyl-5a,6,7,8,9,10-hexahydro- 5H-4-oxa-3,10a,11,13,14- pentaaza-6,9- methanonaphtho[1,8- ab]heptalen-12(13H)-one | 666 [M + H]⁺ |
| 349 | 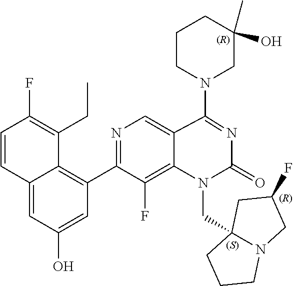 | 7-(8-ethyl-7-fluoro-3- hydroxynaphthalen-1-yl)-8- fluoro-1-(((2R,7aS)-2- fluorotetrahydro-1H-pyrrolizin- 7a(5H)-yl)methyl)-4-((R)-3- hydroxy-3-methylpiperidin-1- yl)pyrido[4,3-d]pyrimidin- 2(1H)-one | 607 [M + H]⁺ |
| 350a1 | 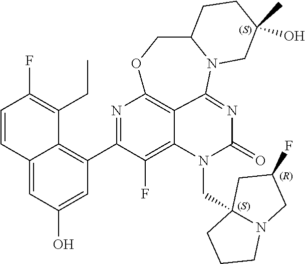 | (8S)-2-(8-ethyl-7-fluoro-3- hydroxynaphthalen-1-yl)-1- fluoro-12-(((2R,7aS)-2- fluorotetrahydro-1H-pyrrolizin- 7a(5H)-yl)methyl)-8-hydroxy-8- methyl-5,5a,6,7,8,9-hexahydro- 4-oxa-3,9a,10,12- tetraazabenzo[4,5]cyclohepta[1, 2,3-de]naphthalen-11(12H)-one Isomer 1 (the first eluting isomer) | 636 [M + H]⁺ |
| 350a2 | 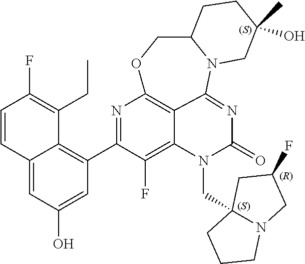 | (8S)-2-(8-ethyl-7-fluoro-3- hydroxynaphthalen-1-yl)-1- fluoro-12-(((2R,7aS)-2- fluorotetrahydro-1H-pyrrolizin- 7a(5H)-yl)methyl)-8-hydroxy-8- methyl-5,5a,6,7,8,9-hexahydro- 4-oxa-3,9a,10,12- tetraazabenzo[4,5]cyclohepta[1, 2,3-de]naphthalen-11(12H)-one Isomer 2 (the second eluting isomer) | 636 [M + H]⁺ |
| 350b1 | 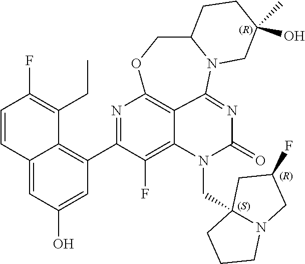 | (8R)-2-(8-ethyl-7-fluoro-3- hydroxynaphthalen-1-yl)-1- fluoro-12-(((2R,7aS)-2- fluorotetrahydro-1H-pyrrolizin- 7a(5H)-yl)methyl)-8-hydroxy-8- methyl-5,5a,6,7,8,9-hexahydro- 4-oxa-3,9a,10,12- tetraazabenzo[4,5]cyclohepta[1, 2,3-de]naphthalen-11(12H)-one Isomer 1 (the first eluting isomer) | 636 [M + H]⁺ |
| 350b2 | 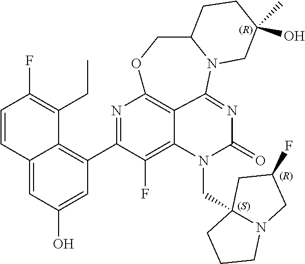 | (8R)-2-(8-ethyl-7-fluoro-3- hydroxynaphthalen-1-yl)-1- fluoro-12-(((2R,7aS)-2- fluorotetrahydro-1H-pyrrolizin- 7a(5H)-yl)methyl)-8-hydroxy-8- methyl-5,5a,6,7,8,9-hexahydro- 4-oxa-3,9a,10,12- tetraazabenzo[4,5]cyclohepta[1, 2,3-de]naphthalen-11(12H)-one Isomer 2 (the second eluting isomer) | 636 [M + H]⁺ |
| 351 | 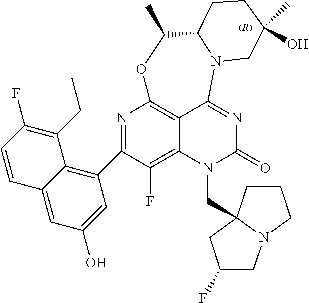 | (5S,5aS,8R)-2-(8-ethyl-7-fluoro- 3-hydroxynaphthalen-1-yl)-1- fluoro-12-(((2R,7aS)-2- fluorotetrahydro-1H-pyrrolizin- 7a(5H)-yl)methyl)-8-hydroxy- 5,8-dimethyl-5,5a,6,7,8,9- hexahydro-4-oxa-3,9a,10,12- tetraazabenzo[4,5]cyclohepta[1, 2,3-de]naphthalen-11(12H)-one | 650 [M + H]⁺ |
| 352 | 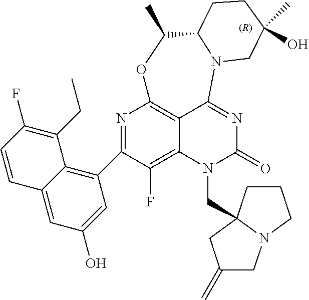 | (5S,5aS,8R)-2-(8-ethyl-7-fluoro- 3-hydroxynaphthalen-1-yl)-1- fluoro-8-hydroxy-5,8-dimethyl- 12-(((S)-2-methylenetetrahydro- 1H-pyrrolizin-7a(5H)- yl)methyl)-5,5a,6,7,8,9- hexahydro-4-oxa-3,9a,10,12- tetraazabenzo[4,5]cyclohepta[1, 2,3-de]naphthalen-11(12H)-one | 644 [M + H]⁺ |
| 353 | 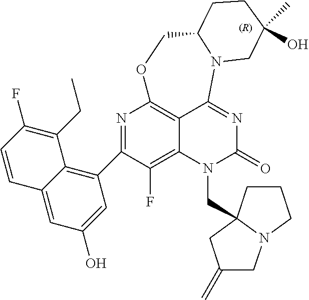 | (5aS,8R)-2-(8-ethyl-7-fluoro-3- hydroxynaphthalen-1-yl)-1- fluoro-8-hydroxy-8-methyl-12- (((S)-2-methylenetetrahydro-1H- pyrrolizin-7a(5H)-yl)methyl)- 5,5a,6,7,8,9-hexahydro-4-oxa- 3,9a,10,12- tetraazabenzo[4,5]cyclohepta[1, 2,3-de]naphthalen-11(12H)-one | 630 [M + H]⁺ |
| 354 | 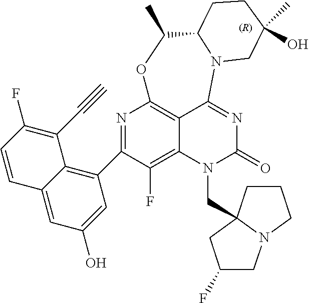 | (5S,5aS,8R)-2-(8-ethynyl-7- fluoro-3-hydroxynaphthalen-1- yl)-1-fluoro-12-(((2R,7aS)-2- fluorotetrahydro-1H-pyrrolizin- 7a(5H)-yl)methyl)-8-hydroxy- 5,8-dimethyl-5,5a,6,7,8,9- hexahydro-4-oxa-3,9a,10,12- tetraazabenzo[4,5]cyclohepta[1, 2,3-de]naphthalen-11(12H)-one | 646 [M + H]⁺ |
| 355 | 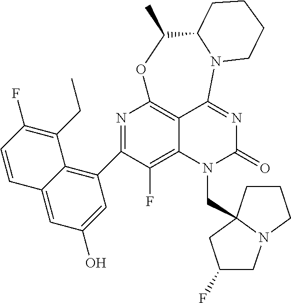 [SMILES: CCc1c(F)ccc2cc(O)cc(-c3nc4c5c(nc(=O)n(C[C@@]67CCCN6C[C@H](F)C7)c5c3F)N3CCCC[C@H]3[C@H](C)O4)c12] | (5S,5aS)-2-(8-ethyl-7-fluoro-3- hydroxynaphthalen-1-yl)-1- fluoro-12-(((2R,7aS)-2- fluorotetrahydro-1H-pyrrolizin- 7a(5H)-yl)methyl)-5-methyl- 5,5a,6,7,8,9-hexahydro-4-oxa- 3,9a,10,12- tetraazabenzo[4,5]cyclohepta[1, 2,3-de]naphthalen-11(12H)-one | 620 [M + H]⁺ |
| 356 | 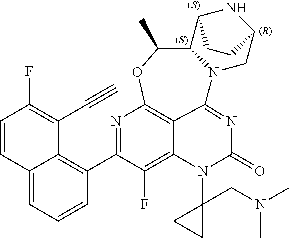 | (5S,5aS,6S,9R)-13-(1- ((dimethylamino)methyl) cyclopropyl)-2-(8-ethynyl-7- fluoronaphthalen-1-yl)-1-fluoro- 5-methyl-5a,6,7,8,9,10- hexahydro-5H-4-oxa- 3,10a,11,13,14-pentaaza-6,9- methanonaphtho[1,8- ab]heptalen-12(13H)-one | 583 [M + H]⁺ |
| 357 | 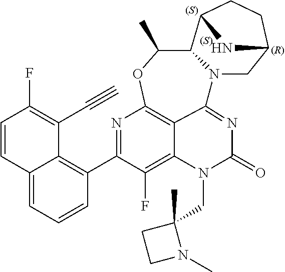 | (5S,5aS,6S,9R)-13-(((R)-1,2- dimethylazetidin-2-yl)methyl)- 2-(8-ethynyl-7- fluoronaphthalen-1-yl)-1-fluoro- 5-methyl-5a,6,7,8,9,10- hexahydro-5H-4-oxa- 3,10a,11,13,14-pentaaza-6,9- methanonaphtho[1,8- ab]heptalen-12(13H)-one | 583 [M + H]⁺ |
| 358 | 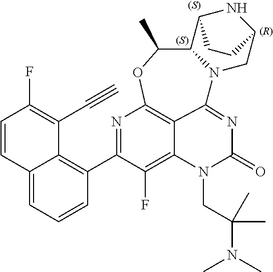 | (5S,5aS,6S,9R)-13-(2- (dimethylamino)-2- methylpropyl)-2-(8-ethynyl-7- fluoronaphthalen-1-yl)-1-fluoro- 5-methyl-5a,6,7,8,9,10- hexahydro-5H-4-oxa- 3,10a,11,13,14-pentaaza-6,9- methanonaphtho[1,8- ab]heptalen-12(13H)-one | 585 [M + H]⁺ |
| 359 | 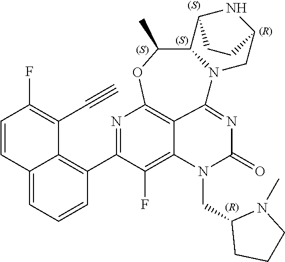 | (5S,5aS,6S,9R)-2-(8-ethynyl-7- fluoronaphthalen-1-yl)-1-fluoro- 5-methyl-13-(((R)-1- methylpyrrolidin-2-yl)methyl)- 5a,6,7,8,9,10-hexahydro-5H-4- oxa-3,10a,11,13,14-pentaaza- 6,9-methanonaphtho[1,8- ab]heptalen-12(13H)-one | 583 [M + H]⁺ |
| 360 | 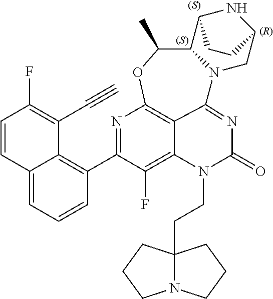 | (5S,5aS,6S,9R)-2-(8-ethynyl-7- fluoronaphthalen-1-yl)-1-fluoro- 5-methyl-13-(2-(tetrahydro-1H- pyrrolizin-7a(5H)-yl)ethyl)- 5a,6,7,8,9,10-hexahydro-5H-4- oxa-3,10a,11,13,14-pentaaza- 6,9-methanonaphtho[1,8- ab]heptalen-12(13H)-one | 623 [M + H]⁺ |
| 361 | 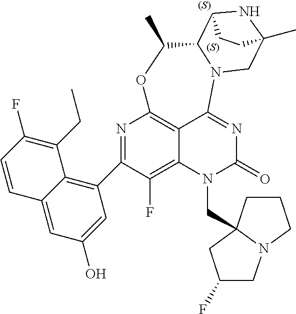 | (5S,5aS,6S,9R)-2-(8-ethyl-7- fluoro-3-hydroxynaphthalen-1- yl)-1-fluoro-13-(((2R,7aS)-2- fluorotetrahydro-1H-pyrrolizin- 7a(5H)-yl)methyl)-5,9-dimethyl- 5a,6,7,8,9,10-hexahydro-5H-4- oxa-3,10a,11,13,14-pentaaza- 6,9-methanonaphtho[1,8- ab]heptalen-12(13H)-one | 661 [M + H]⁺ |
| 362 | 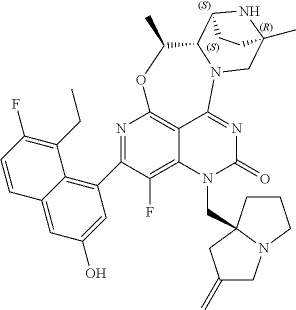 | (5S,5aS,6S,9R)-2-(8-ethyl-7- fluoro-3-hydroxynaphthalen-1- yl)-1-fluoro-5,9-dimethyl-13- (((S)-2-methylenetetrahydro-1H- pyrrolizin-7a(5H)-yl)methyl)- 5a,6,7,8,9,10-hexahydro-5H-4- oxa-3,10a,11,13,14-pentaaza- 6,9-methanonaphtho[1,8- ab]heptalen-12(13H)-one | 655 [M + H]⁺ |
| 363 | 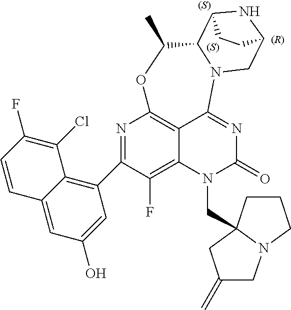 | (5S,5aS,6S,9R)-2-(8-chloro-7- fluoro-3-hydroxynaphthalen-1- yl)-1-fluoro-5-methyl-13-(((S)- 2-methylenetetrahydro-1H- pyrrolizin-7a(5H)-yl)methyl)- 5a,6,7,8,9,10-hexahydro-5H-4- oxa-3,10a,11,13,14-pentaaza- 6,9-methanonaphtho[1,8- ab]heptalen-12(13H)-one | 647 [M + H]⁺ |
| 364 | 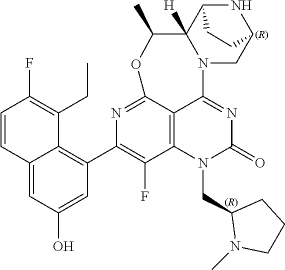 | (8S,8aS,9S,12R)-5-(8-ethyl-7- fluoro-3-hydroxynaphthalen-1- yl)-4-fluoro-8-methyl-3-(((R)-1- methylpyrrolidin-2-yl)methyl)- 8a,9,10,11,12,13-hexahydro-8H- 7-oxa-1,3,6,13a,14-pentaaza- 9,12-methanonaphtho[1,8- ab]heptalen-2(3H)-one | 603 [M + H]⁺ |
| 366 | 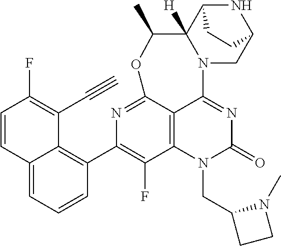 [SMILES: C#Cc1c(F)ccc2cccc(-c3nc4c5c(nc(=O)n(C[C@H]6CCN6C)c5c3F)N3C[C@H]5CC[C@H](N5)[C@H]3[C@H](C)O4)c12] | (8S,8aS,9S,12R)-5-(8-ethynyl-7- fluoronaphthalen-1-yl)-4-fluoro- 8-methyl-3-(((R)-1- methylazetidin-2-yl)methyl)- 8a,9,10,11,12,13-hexahydro-8H- 7-oxa-1,3,6,13a,14-pentaaza- 9,12-methanonaphtho[1,8- ab]heptalen-2(3H)-one | 569 [M + H]⁺ |
| 367 | 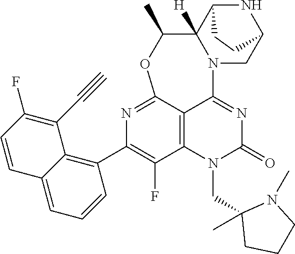 [SMILES: C#Cc1c(F)ccc2cccc(-c3nc4c5c(nc(=O)n(C[C@@]6(C)CCCN6C)c5c3F)N3C[C@H]5CC[C@H](N5)[C@H]3[C@H](C)O4)c12] | (8S,8aS,9S,12R)-3-(((R)-1,2- dimethylpyrrolidin-2- yl)methyl)-5-(8-ethynyl-7- fluoronaphthalen-1-yl)-4-fluoro- 8-methyl-8a,9,10,11,12,13- hexahydro-8H-7-oxa- 1,3,6,13a,14-pentaaza-9,12- methanonaphtho[1,8- ab]heptalen-2(3H)-one | 597 [M + H]⁺ |
| 368 | 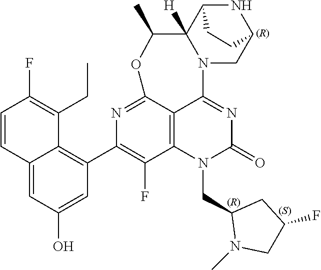 | (8S,8aS,9S,12R)-5-(8-ethyl-7- fluoro-3-hydroxynaphthalen-1- yl)-4-fluoro-3-(((2R,4S)-4- fluoro-1-methylpyrrolidin-2- yl)methyl)-8-methyl- 8a,9,10,11,12,13-hexahydro-8H- 7-oxa-1,3,6,13a,14-pentaaza- 9,12-methanonaphtho[1,8- ab]heptalen-2(3H)-one | 621 [M + H]⁺ |
| 369 | 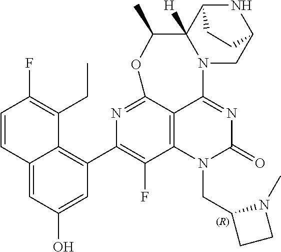 | (8S,8aS,9S,12R)-5-(8-ethyl-7- fluoro-3-hydroxynaphthalen-1- yl)-4-fluoro-8-methyl-3-(((R)-1- methylazetidin-2-yl)methyl)- 8a,9,10,11,12,13-hexahydro-8H- 7-oxa-1,3,6,13a,14-pentaaza- 9,12-methanonaphtho[1,8- ab]heptalen-2(3H)-one | 589 [M + H]⁺ |
| 370 | 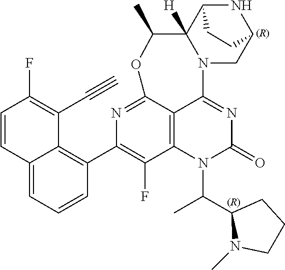 | (8S,8aS,9S,12R)-5-(8-ethynyl-7- fluoronaphthalen-1-yl)-4-fluoro- 8-methyl-3-(1-((R)-1- methylpyrrolidin-2-yl)ethyl)- 8a,9,10,11,12,13-hexahydro-8H- 7-oxa-1,3,6,13a,14-pentaaza- 9,12-methanonaphtho[1,8- ab]heptalen-2(3H)-one | 597 [M + H]⁺ |
| 371a | 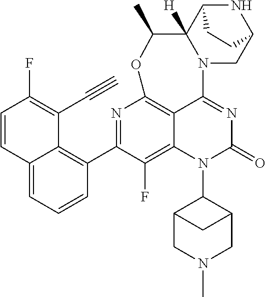 [SMILES: C#Cc1c(F)ccc2cccc(-c3nc4c5c(nc(=O)n(C6C7CC6CN(C)C7)c5c3F)N3C[C@H]5CC[C@H](N5)[C@H]3[C@H](C)O4)c12] | (5S,5aS,6S,9R)-2-(8-ethynyl-7- fluoronaphthalen-1-yl)-1-fluoro- 5-methyl-13-(3-methyl-3- azabicyclo[3.1.1]heptan-6-yl)- 5a,6,7,8,9,10-hexahydro-5H-4- oxa-3,10a,11,13,14-pentaaza- 6,9-methanonaphtho[1,8- ab]272eptalene-12(13H)-one Isomer 1 | 595 [M + H]⁺ |
| 371b | 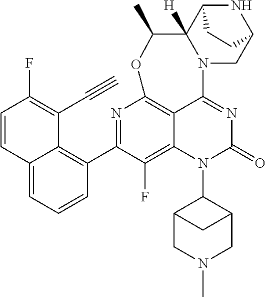 [SMILES: C#Cc1c(F)ccc2cccc(-c3nc4c5c(nc(=O)n(C6C7CC6CN(C)C7)c5c3F)N3C[C@H]5CC[C@H](N5)[C@H]3[C@H](C)O4)c12] | (5S,5aS,6S,9R)-2-(8-ethynyl-7- fluoronaphthalen-1-yl)-1-fluoro- 5-methyl-13-(3-methyl-3- azabicyclo[3.1.1]heptan-6-yl)- 5a,6,7,8,9,10-hexahydro-5H-4- oxa-3,10a,11,13,14-pentaaza- 6,9-methanonaphtho[1,8- ab]heptalen-12(13H)-one Isomer 2 | 595 [M + H]⁺ |
| 372a | 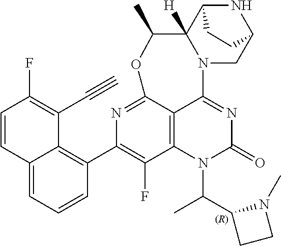 | (8S,8aS,9S,12R)-5-(8-ethynyl-7- fluoronaphthalen-1-yl)-4-fluoro- 8-methyl-3-(1-((R)-1- methylazetidin-2-yl)ethyl)- 8a,9,10,11,12,13-hexahydro-8H- 7-oxa-1,3,6,13a,14-pentaaza- 9,12-methanonaphtho[1,8- ab]heptalen-2(3H)-one Isomer 1 | 583 [M + H]⁺ |
| 372b | 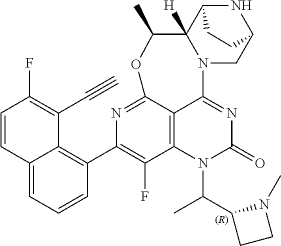 | (8S,8aS,9S,12R)-5-(8-ethynyl-7- fluoronaphthalen-1-yl)-4-fluoro- 8-methyl-3-(1-((R)-1- methylazetidin-2-yl)ethyl)- 8a,9,10,11,12,13-hexahydro-8H- 7-oxa-1,3,6,13a,14-pentaaza- 9,12-methanonaphtho[1,8- ab]heptalen-2(3H)-one Isomer 2 | 583 [M + H]⁺ |
| 373a | 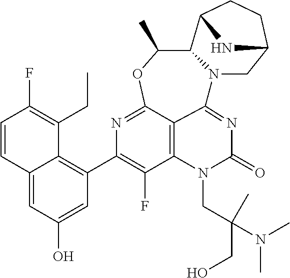 [SMILES: CCc1c(F)ccc2cc(O)cc(-c3nc4c5c(nc(=O)n(CC(C)(CO)N(C)C)c5c3F)N3C[C@H]5CC[C@H](N5)[C@H]3[C@H](C)O4)c12] | (5S,5aS,6S,9R)-13-(2- (dimethylamino)-3-hydroxy-2- methylpropyl)-2-(8-ethyl-7- fluoro-3-hydroxynaphthalen-1- yl)-1-fluoro-5-methyl- 5a,6,7,8,9,10-hexahydro-5H-4- oxa-3,10a,11,13,14-pentaaza- 6,9-methanonaphtho[1,8- ab]heptalen-12(13H)-one Isomer 1 | 621 [M + H]⁺ |
| 373b | 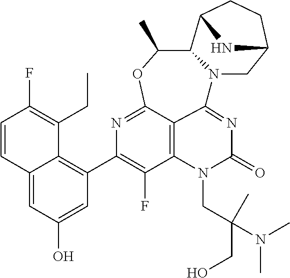 [SMILES: CCc1c(F)ccc2cc(O)cc(-c3nc4c5c(nc(=O)n(CC(C)(CO)N(C)C)c5c3F)N3C[C@H]5CC[C@H](N5)[C@H]3[C@H](C)O4)c12] | (5S,5aS,6S,9R)-13-(2- (dimethylamino)-3-hydroxy-2- methylpropyl)-2-(8-ethyl-7- fluoro-3-hydroxynaphthalen-1- yl)-1-fluoro-5-methyl- 5a,6,7,8,9,10-hexahydro-5H-4- oxa-3,10a,11,13,14-pentaaza- 6,9-methanonaphtho[1,8- ab]heptalen-12(13H)-one Isomer 2 | 621 [M + H]⁺ |
| 374 | 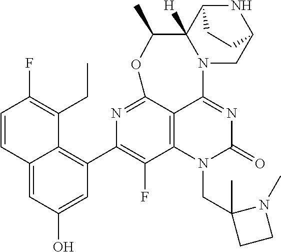 [SMILES: CCc1c(F)ccc2cc(O)cc(-c3nc4c5c(nc(=O)n(CC6(C)CCN6C)c5c3F)N3C[C@H]5CC[C@H](N5)[C@H]3[C@H](C)O4)c12] | (8S,8aS,9S,12R)-3-((1,2- dimethylazetidin-2-yl)methyl)- 5-(8-ethyl-7-fluoro-3- hydroxynaphthalen-1-yl)-4- fluoro-8-methyl- 8a,9,10,11,12,13-hexahydro-8H- 7-oxa-1,3,6,13a,14-pentaaza- 9,12-methanonaphtho[1,8- ab]heptalen-2(3H)-one | 603 [M + H]⁺ |
| 375 | 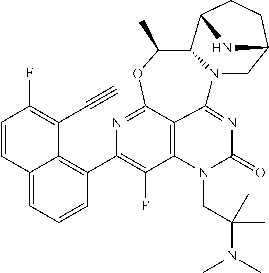 [SMILES: C#Cc1c(F)ccc2cccc(-c3nc4c5c(nc(=O)n(CC(C)(C)N(C)C)c5c3F)N3C[C@H]5CC[C@H](N5)[C@H]3[C@H](C)O4)c12] | (5S,5aS,6S,9R)-13-(2- (dimethylamino)-2- methylpropyl)-2-(8-ethynyl-7- fluoronaphthalen-1-yl)-1-fluoro- 5-methyl-5a,6,7,8,9,10- hexahydro-5H-4-oxa- 3,10a,11,13,14-pentaaza-6,9- methanonaphtho[1,8- ab]heptalen-12(13H)-one | 585 [M + H]⁺ |
| 376a | 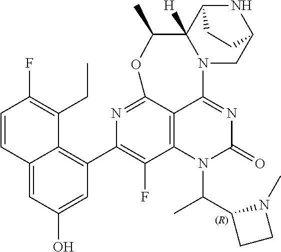 | (8S,8aS,9S,12R)-5-(8-ethyl-7- fluoro-3-hydroxynaphthalen-1- yl)-4-fluoro-8-methyl-3-(1-(®-1- methylazetidin-2-yl)ethyl)- 8a,9,10,11,12,13-hexahydro-8H- 7-oxa-1,3,6,13a,14-pentaaza- 9,12-methanonaphtho[1,8- ab]275eptalene-2(3H)-one Isomer 1 | 603 [M + H]⁺ |
| 376b | 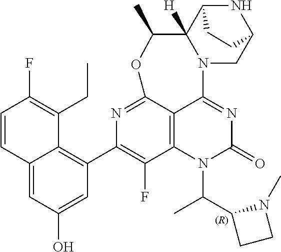 | (8S,8aS,9S,12R)-5-(8-ethyl-7- fluoro-3-hydroxynaphthalen-1- yl)-4-fluoro-8-methyl-3-(1-((R)- 1-methylazetidin-2-yl)ethyl)- 8a,9,10,11,12,13-hexahydro-8H- 7-oxa-1,3,6,13a,14-pentaaza- 9,12-methanonaphtho[1,8- ab]heptalen-2(3H)-one Isomer 2 | 603 [M + H]⁺ |
| 377 | 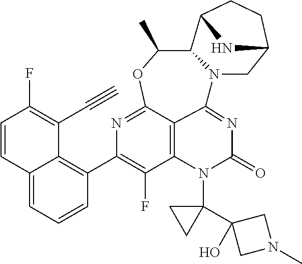 [SMILES: C#Cc1c(F)ccc2cccc(-c3nc4c5c(nc(=O)n(C6(C7(O)CN(C)C7)CC6)c5c3F)N3C[C@H]5CC[C@H](N5)[C@H]3[C@H](C)O4)c12] | (8S,8aS,9S,12R)-5-(8-ethynyl-7- fluoronaphthalen-1-yl)-4-fluoro- 8-methyl-3-((R)-1-((S)-1- methylazetidin-2-yl)ethyl)- 8a,9,10,11,12,13-hexahydro-8H- 7-oxa-1,3,6,13a,14-pentaaza- 9,12-methanonaphtho[1,8- ab]heptalen-2(3H)-one | 611 [M + H]⁺ |
| 378 | 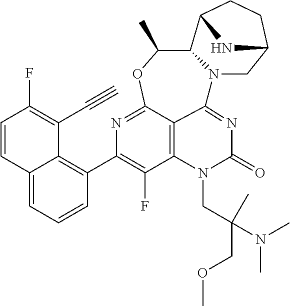 [SMILES: C#Cc1c(F)ccc2cccc(-c3nc4c5c(nc(=O)n(CC(C)(COC)N(C)C)c5c3F)N3C[C@H]5CC[C@H](N5)[C@H]3[C@H](C)O4)c12] | (8S,8aS,9S,12R)-5-(8-ethynyl-7- fluoronaphthalen-1-yl)-4-fluoro- 8-methyl-3-((S)-1-((S)-1- methylazetidin-2-yl)ethyl)- 8a,9,10,11,12,13-hexahydro-8H- 7-oxa-1,3,6,13a,14-pentaaza- 9,12-methanonaphtho[1,8- ab]heptalen-2(3H)-one | 615 [M + H]⁺ |
| 379 | 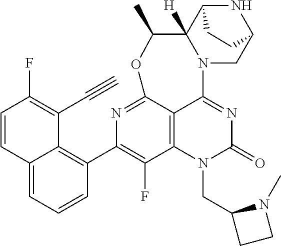 [SMILES: C#Cc1c(F)ccc2cccc(-c3nc4c5c(nc(=O)n(C[C@@H]6CCN6C)c5c3F)N3C[C@H]5CC[C@H](N5)[C@H]3[C@H](C)O4)c12] | (8S,8aS,9S,12R)-3-(((R)-1,2- dimethylazetidin-2-yl)methyl)- 5-(8-ethyl-7-fluoro-3- hydroxynaphthalen-1-yl)-4- fluoro-8-methyl- 8a,9,10,11,12,13-hexahydro-8H- 7-oxa-1,3,6,13a,14-pentaaza- 9,12-methanonaphtho[1,8- ab]heptalen-2(3H)-one | 569 [M + H]⁺ |
| 380 | 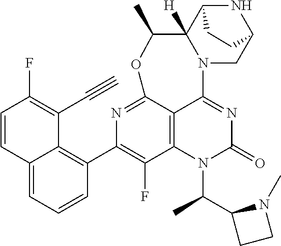 [SMILES: C#Cc1c(F)ccc2cccc(-c3nc4c5c(nc(=O)n([C@H](C)[C@@H]6CCN6C)c5c3F)N3C[C@H]5CC[C@H](N5)[C@H]3[C@H](C)O4)c12] | (8S,8aS,9S,12R)-5-(8-ethynyl-7- fluoronaphthalen-1-yl)-4-fluoro- 8-methyl-3-((R)-1-((S)-1- methylazetidin-2-yl)ethyl)- 8a,9,10,11,12,13-hexahydro-8H- 7-oxa-1,3,6,13a,14-pentaaza- 9,12-methanonaphtho[1,8- ab]heptalen-2(3H)-one | 583 [M + H]⁺ |
| 381 | 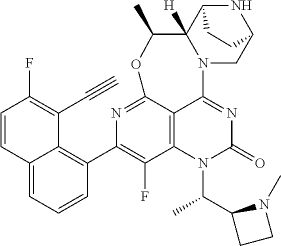 [SMILES: C#Cc1c(F)ccc2cccc(-c3nc4c5c(nc(=O)n([C@@H](C)[C@@H]6CCN6C)c5c3F)N3C[C@H]5CC[C@H](N5)[C@H]3[C@H](C)O4)c12] | (8S,8aS,9S,12R)-5-(8-ethynyl-7- fluoronaphthalen-1-yl)-4-fluoro- 8-methyl-3-((S)-1-((S)-1- methylazetidin-2-yl)ethyl)- 8a,9,10,11,12,13-hexahydro-8H- 7-oxa-1,3,6,13a,14-pentaaza- 9,12-methanonaphtho[1,8- ab]heptalen-2(3H)-one | 583 [M + H]⁺ |
| 382 | 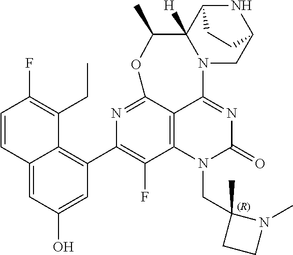 | (8S,8aS,9S,12R)-3-(((R)-1,2- dimethylazetidin-2-yl)methyl)- 5-(8-ethyl-7-fluoro-3- hydroxynaphthalen-1-yl)-4- fluoro-8-methyl- 8a,9,10,11,12,13-hexahydro-8H- 7-oxa-1,3,6,13a,14-pentaaza- 9,12-methanonaphtho[1,8- ab]heptalen-2(3H)-one | 603 [M + H]⁺ |
| 383a | 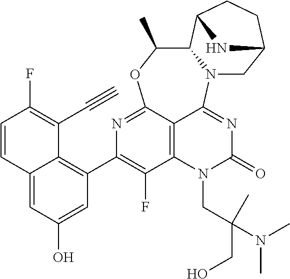 [SMILES: C#Cc1c(F)ccc2cc(O)cc(-c3nc4c5c(nc(=O)n(CC(C)(CO)N(C)C)c5c3F)N3C[C@H]5CC[C@H](N5)[C@H]3[C@H](C)O4)c12] | (5S,5aS,6S,9R)-13-(2- (dimethylamino)-3-hydroxy-2- methylpropyl)-2-(8-ethynyl-7- fluoro-3-hydroxynaphthalen-1- yl)-1-fluoro-5-methyl- 5a,6,7,8,9,10-hexahydro-5H-4- oxa-3,10a,11,13,14-pentaaza- 6,9-methanonaphtho[1,8- ab]heptalen-12(13H)-one Isomer 1 (the first eluting isomer) | 617 [M + H]⁺ |
| 383b | 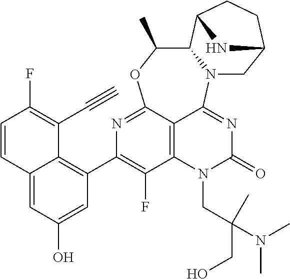 [SMILES: C#Cc1c(F)ccc2cc(O)cc(-c3nc4c5c(nc(=O)n(CC(C)(CO)N(C)C)c5c3F)N3C[C@H]5CC[C@H](N5)[C@H]3[C@H](C)O4)c12] | (5S,5aS,6S,9R)-13-(2- (dimethylamino)-3-hydroxy-2- methylpropyl)-2-(8-ethynyl-7- fluoro-3-hydroxynaphthalen-1- yl)-1-fluoro-5-methyl- 5a,6,7,8,9,10-hexahydro-5H-4- oxa-3,10a,11,13,14-pentaaza- 6,9-methanonaphtho[1,8- ab]heptalen-12(13H)-one Isomer 2 (the second eluting isomer) | 617 [M + H]⁺ |
| 384a | 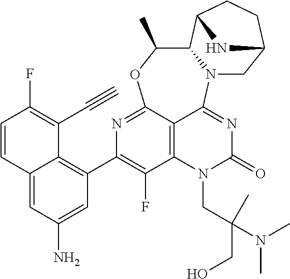 [SMILES: C#Cc1c(F)ccc2cc(N)cc(-c3nc4c5c(nc(=O)n(CC(C)(CO)N(C)C)c5c3F)N3C[C@H]5CC[C@H](N5)[C@H]3[C@H](C)O4)c12] | (5S,5aS,6S,9R)-2-(3-amino-8- ethynyl-7-fluoronaphthalen-1- yl)-13-(2-(dimethylamino)-3- hydroxy-2-methylpropyl)-1- fluoro-5-methyl-5a,6,7,8,9,10- hexahydro-5H-4-oxa- 3,10a,11,13,14-pentaaza-6,9- methanonaphtho[1,8- ab]heptalen-12(13H)-one Isomer 1 (the first eluting isomer) | 616 [M + H]⁺ |
| 384b | 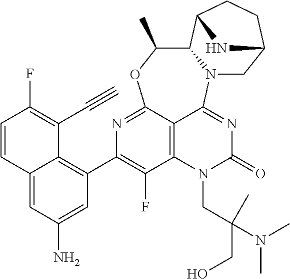 [SMILES: C#Cc1c(F)ccc2cc(N)cc(-c3nc4c5c(nc(=O)n(CC(C)(CO)N(C)C)c5c3F)N3C[C@H]5CC[C@H](N5)[C@H]3[C@H](C)O4)c12] | (5S,5aS,6S,9R)-2-(3-amino-8- ethynyl-7-fluoronaphthalen-1- yl)-13-(2-(dimethylamino)-3- hydroxy-2-methylpropyl)-1- fluoro-5-methyl-5a,6,7,8,9,10- hexahydro-5H-4-oxa- 3,10a,11,13,14-pentaaza-6,9- methanonaphtho[1,8- ab]heptalen-12(13H)-one Isomer 2 (the second eluting isomer) | 616 [M + H]⁺ |
| 385 | 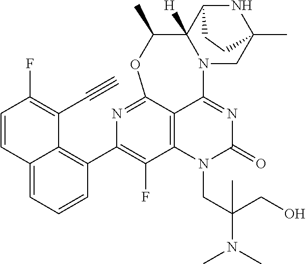 [SMILES: C#Cc1c(F)ccc2cccc(-c3nc4c5c(nc(=O)n(CC(C)(CO)N(C)C)c5c3F)N3C[C@@]5(C)CC[C@H](N5)[C@H]3[C@H](C)O4)c12] | (8S,8aS,9S,12R)-3-(2- (dimethylamino)-3-hydroxy-2- methylpropyl)-5-(8-ethynyl-7- fluoronaphthalen-1-yl)-4-fluoro- 8,12-dimethyl-8a,9,10,11,12,13- hexahydro-8H-7-oxa- 1,3,6,13a,14-pentaaza-9,12- methanonaphtho[1,8- ab]heptalen-2(3H)-one | 615 [M + H]⁺ |
| 386 | 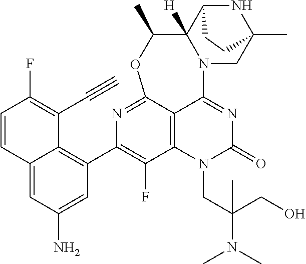 [SMILES: C#Cc1c(F)ccc2cc(N)cc(-c3nc4c5c(nc(=O)n(CC(C)(CO)N(C)C)c5c3F)N3C[C@@]5(C)CC[C@H](N5)[C@H]3[C@H](C)O4)c12] | (8S,8aS,9S,12R)-5-(3-amino-8- ethynyl-7-fluoronaphthalen-1- yl)-3-(2-(dimethylamino)-3- hydroxy-2-methylpropyl)-4- fluoro-8,12-dimethyl- 8a,9,10,11,12,13-hexahydro-8H- 7-oxa-1,3,6,13a,14-pentaaza- 9,12-methanonaphtho[1,8- ab]heptalen-2(3H)-one | 630 [M + H]⁺ |
| 387 | 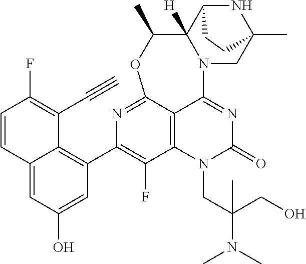 [SMILES: C#Cc1c(F)ccc2cc(O)cc(-c3nc4c5c(nc(=O)n(CC(C)(CO)N(C)C)c5c3F)N3C[C@@]5(C)CC[C@H](N5)[C@H]3[C@H](C)O4)c12] | (8S,8aS,9S,12R)-3-(2- (dimethylamino)-3-hydroxy-2- methylpropyl)-5-(8-ethynyl-7- fluoro-3-hydroxynaphthalen-1- yl)-4-fluoro-8,12-dimethyl- 8a,9,10,11,12,13-hexahydro-8H- 7-oxa-1,3,6,13a,14-pentaaza- 9,12-methanonaphtho[1,8- ab]heptalen-2(3H)-one | 631 [M + H]⁺ |
| 388 | 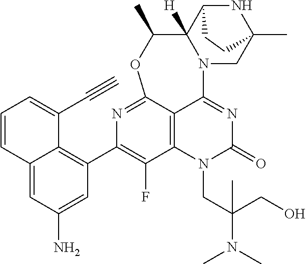 [SMILES: C#Cc1cccc2cc(N)cc(-c3nc4c5c(nc(=O)n(CC(C)(CO)N(C)C)c5c3F)N3C[C@@]5(C)CC[C@H](N5)[C@H]3[C@H](C)O4)c12] | (8S,8aS,9S,12R)-5-(3-amino-8- ethynylnaphthalen-1-yl)-3-(2- (dimethylamino)-3-hydroxy-2- methylpropyl)-4-fluoro-8,12- dimethyl-8a,9,10,11,12,13- hexahydro-8H-7-oxa- 1,3,6,13a,14-pentaaza-9,12- methanonaphtho[1,8- ab]heptalen-2(3H)-one | 612 [M + H]⁺ |
| 389 | 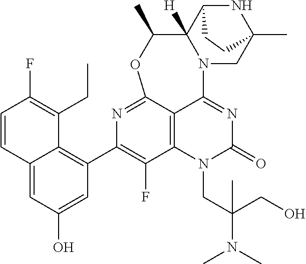 [SMILES: CCc1c(F)ccc2cc(O)cc(-c3nc4c5c(nc(=O)n(CC(C)(CO)N(C)C)c5c3F)N3C[C@@]5(C)CC[C@H](N5)[C@H]3[C@H](C)O4)c12] | (8S,8aS,9S,12R)-3-(2- (dimethylamino)-3-hydroxy-2- methylpropyl)-5-(8-ethyl-7- fluoro-3-hydroxynaphthalen-1- yl)-4-fluoro-8,12-dimethyl- 8a,9,10,11,12,13-hexahydro-8H- 7-oxa-1,3,6,13a,14-pentaaza- 9,12-methanonaphtho[1,8- ab]heptalen-2(3H)-one | 635 [M + H]⁺ |
| 390 | 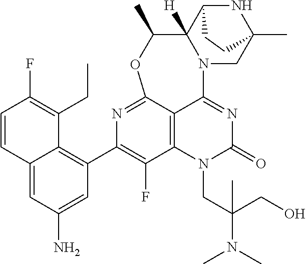 [SMILES: CCc1c(F)ccc2cc(N)cc(-c3nc4c5c(nc(=O)n(CC(C)(CO)N(C)C)c5c3F)N3C[C@@]5(C)CC[C@H](N5)[C@H]3[C@H](C)O4)c12] | (8S,8aS,9S,12R)-5-(3-amino-8- ethyl-7-fluoronaphthalen-1-yl)- 3-(2-(dimethylamino)-3- hydroxy-2-methylpropyl)-4- fluoro-8,12-dimethyl- 8a,9,10,11,12,13-hexahydro-8H- 7-oxa-1,3,6,13a,14-pentaaza- 9,12-methanonaphtho[1,8- ab]heptalen-2(3H)-one | 634 [M + H]⁺ |
| 391 | 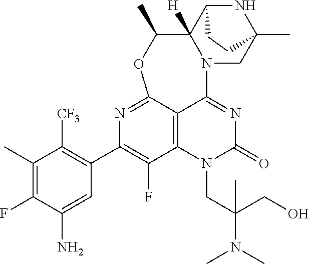 [SMILES: Cc1c(F)c(N)cc(-c2nc3c4c(nc(=O)n(CC(C)(CO)N(C)C)c4c2F)N2C[C@@]4(C)CC[C@H](N4)[C@H]2[C@H](C)O3)c1C(F)(F)F] | (8S,8aS,9S,12R)-5-(5-amino-4- fluoro-3-methyl-2- (trifluoromethyl)phenyl)-3-(2- (dimethylamino)-3-hydroxy-2- methylpropyl)-4-fluoro-8,12- dimethyl-8a,9,10,11,12,13- hexahydro-8H-7-oxa- 1,3,6,13a,14-pentaaza-9,12- methanonaphtho[1,8- ab]heptalen-2(3H)-one | 638 [M + H]⁺ |
| 392 | 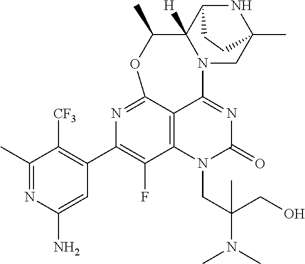 [SMILES: Cc1nc(N)cc(-c2nc3c4c(nc(=O)n(CC(C)(CO)N(C)C)c4c2F)N2C[C@@]4(C)CC[C@H](N4)[C@H]2[C@H](C)O3)c1C(F)(F)F] | (8S,8aS,9S,12R)-5-(6-amino-2- methyl-3- (trifluoromethyl)pyridin-4-yl)-3- (2-(dimethylamino)-3-hydroxy- 2-methylpropyl)-4-fluoro-8,12- dimethyl-8a,9,10,11,12,13- hexahydro-8H-7-oxa- 1,3,6,13a,14-pentaaza-9,12- methanonaphtho[1,8- ab]heptalen-2(3H)-one | 621 [M + H]⁺ |
| 393 | 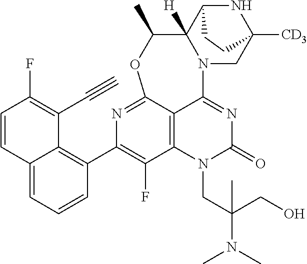 [SMILES: [2H]C([2H])([2H])[C@]12CC[C@H](N1)[C@H]1[C@H](C)Oc3nc(-c4cccc5ccc(F)c(C#C)c45)c(F)c4c3c(nc(=O)n4CC(C)(CO)N(C)C)N1C2] | (8S,8aS,9S,12R)-3-(2- (dimethylamino)-3-hydroxy-2- methylpropyl)-5-(8-ethynyl-7- fluoronaphthalen-1-yl)-4-fluoro- 8-methyl-12-(methyl-d3)- 8a,9,10,11,12,13-hexahydro-8H- 7-oxa-1,3,6,13a,14-pentaaza- 9,12-methanonaphtho[1,8- ab]heptalen-2(3H)-one | 618 [M + H]⁺ |
| 394 | 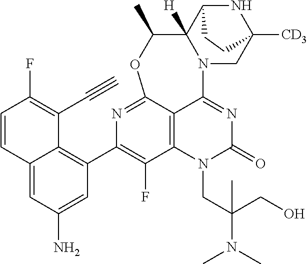 [SMILES: [2H]C([2H])([2H])[C@]12CC[C@H](N1)[C@H]1[C@H](C)Oc3nc(-c4cc(N)cc5ccc(F)c(C#C)c45)c(F)c4c3c(nc(=O)n4CC(C)(CO)N(C)C)N1C2] | (8S,8aS,9S,12R)-5-(3-amino-8- ethynyl-7-fluoronaphthalen-1- yl)-3-(2-(dimethylamino)-3- hydroxy-2-methylpropyl)-4- fluoro-8-methyl-12-(methyl-d3)- 8a,9,10,11,12,13-hexahydro-8H- 7-oxa-1,3,6,13a,14-pentaaza- 9,12-methanonaphtho[1,8- ab]heptalen-2(3H)-one | 633 [M + H]⁺ |
| 395 | 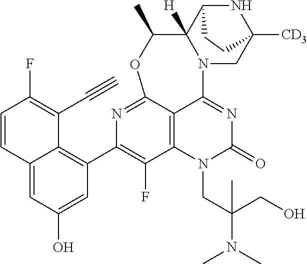 [SMILES: [2H]C([2H])([2H])[C@]12CC[C@H](N1)[C@H]1[C@H](C)Oc3nc(-c4cc(O)cc5ccc(F)c(C#C)c45)c(F)c4c3c(nc(=O)n4CC(C)(CO)N(C)C)N1C2] | (8S,8aS,9S,12R)-3-(2- (dimethylamino)-3-hydroxy-2- methylpropyl)-5-(8-ethynyl-7- fluoro-3-hydroxynaphthalen-1- yl)-4-fluoro-8-methyl-12- (methyl-d3)-8a,9,10,11,12,13- hexahydro-8H-7-oxa- 1,3,6,13a,14-pentaaza-9,12- methanonaphtho[1,8- ab]heptalen-2(3H)-one | 634 [M + H]⁺ |
| 396 | 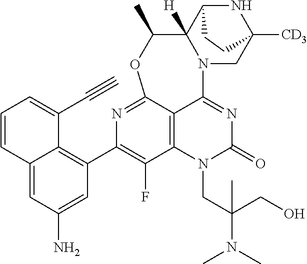 [SMILES: [2H]C([2H])([2H])[C@]12CC[C@H](N1)[C@H]1[C@H](C)Oc3nc(-c4cc(N)cc5cccc(C#C)c45)c(F)c4c3c(nc(=O)n4CC(C)(CO)N(C)C)N1C2] | (8S,8aS,9S,12R)-5-(3-amino-8- ethynylnaphthalen-1-yl)-3-(2- (dimethylamino)-3-hydroxy-2- methylpropyl)-4-fluoro-8- methyl-12-(methyl-d3)- 8a,9,10,11,12,13-hexahydro-8H- 7-oxa-1,3,6,13a,14-pentaaza- 9,12-methanonaphtho[1,8- ab]heptalen-2(3H)-one | 615 [M + H]⁺ |
| 397 | 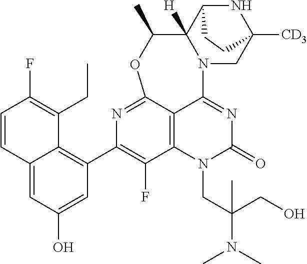 [SMILES: [2H]C([2H])([2H])[C@]12CC[C@H](N1)[C@H]1[C@H](C)Oc3nc(-c4cc(O)cc5ccc(F)c(CC)c45)c(F)c4c3c(nc(=O)n4CC(C)(CO)N(C)C)N1C2] | (8S,8aS,9S,12R)-3-(2- (dimethylamino)-3-hydroxy-2- methylpropyl)-5-(8-ethyl-7- fluoro-3-hydroxynaphthalen-1- yl)-4-fluoro-8-methyl-12- (methyl-d3)-8a,9,10,11,12,13- hexahydro-8H-7-oxa- 1,3,6,13a,14-pentaaza-9,12- methanonaphtho[1,8- ab]heptalen-2(3H)-one | 638 [M + H]⁺ |
| 398 | 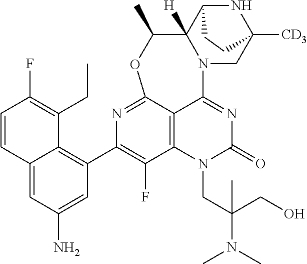 [SMILES: [2H]C([2H])([2H])[C@]12CC[C@H](N1)[C@H]1[C@H](C)Oc3nc(-c4cc(N)cc5ccc(F)c(CC)c45)c(F)c4c3c(nc(=O)n4CC(C)(CO)N(C)C)N1C2] | (8S,8aS,9S,12R)-5-(3-amino-8- ethyl-7-fluoronaphthalen-1-yl)- 3-(2-(dimethylamino)-3- hydroxy-2-methylpropyl)-4- fluoro-8-methyl-12-(methyl-d3)- 8a,9,10,11,12,13-hexahydro-8H- 7-oxa-1,3,6,13a,14-pentaaza- 9,12-methanonaphtho[1,8- ab]heptalen-2(3H)-one | 637 [M + H]⁺ |
| 399 | 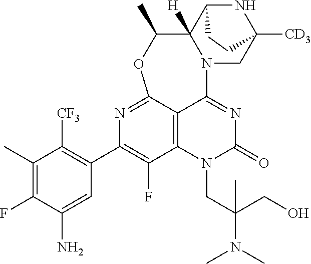 [SMILES: [2H]C([2H])([2H])[C@]12CC[C@H](N1)[C@H]1[C@H](C)Oc3nc(-c4cc(N)c(F)c(C)c4C(F)(F)F)c(F)c4c3c(nc(=O)n4CC(C)(CO)N(C)C)N1C2] | (8S,8aS,9S,12R)-5-(5-amino-4- fluoro-3-methyl-2- (trifluoromethyl)phenyl)-3-(2- (dimethylamino)-3-hydroxy-2- methylpropyl)-4-fluoro-8- methyl-12-(methyl-d3)- 8a,9,10,11,12,13-hexahydro-8H- 7-oxa-1,3,6,13a,14-pentaaza- 9,12-methanonaphtho[1,8- ab]heptalen-2(3H)-one | 641 [M + H]⁺ |
| 400 | 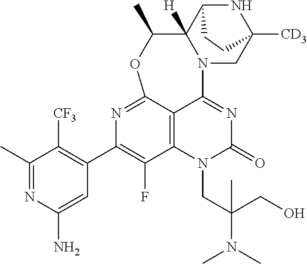 [SMILES: [2H]C([2H])([2H])[C@]12CC[C@H](N1)[C@H]1[C@H](C)Oc3nc(-c4cc(N)nc(C)c4C(F)(F)F)c(F)c4c3c(nc(=O)n4CC(C)(CO)N(C)C)N1C2] | (8S,8aS,9S,12R)-5-(6-amino-2- methyl-3- (trifluoromethyl)pyridin-4-yl)-3- (2-(dimethylamino)-3-hydroxy- 2-methylpropyl)-4-fluoro-8- methyl-12-(methyl-d3)- 8a,9,10,11,12,13-hexahydro-8H- 7-oxa-1,3,6,13a,14-pentaaza- 9,12-methanonaphtho[1,8- ab]heptalen-2(3H)-one | 624 [M + H]⁺ |
| 401 | 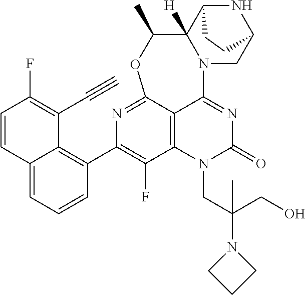 [SMILES: C#Cc1c(F)ccc2cccc(-c3nc4c5c(nc(=O)n(CC(C)(CO)N6CCC6)c5c3F)N3C[C@H]5CC[C@H](N5)[C@H]3[C@H](C)O4)c12] | (8S,8aS,9S,12R)-3-(2-(azetidin- 1-yl)-3-hydroxy-2- methylpropyl)-5-(8-ethynyl-7- fluoronaphthalen-1-yl)-4-fluoro- 8-methyl-8a,9,10,11,12,13- hexahydro-8H-7-oxa- 1,3,6,13a,14-pentaaza-9,12- methanonaphtho[1,8- ab]heptalen-2(3H)-one | 613 [M + H]⁺ |
| 402 | 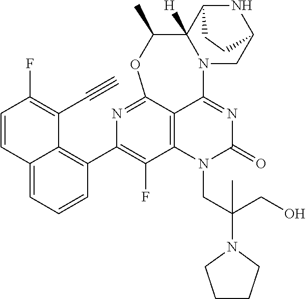 [SMILES: C#Cc1c(F)ccc2cccc(-c3nc4c5c(nc(=O)n(CC(C)(CO)N6CCCC6)c5c3F)N3C[C@H]5CC[C@H](N5)[C@H]3[C@H](C)O4)c12] | (8S,8aS,9S,12R)-5-(8-ethynyl-7- fluoronaphthalen-1-yl)-4-fluoro- 3-(3-hydroxy-2-methyl-2- (pyrrolidin-1-yl)propyl)-8- methyl-8a,9,10,11,12,13- hexahydro-8H-7-oxa- 1,3,6,13a,14-pentaaza-9,12- methanonaphtho[1,8- ab]heptalen-2(3H)-one | 627 [M + H]⁺ |
| 403 | 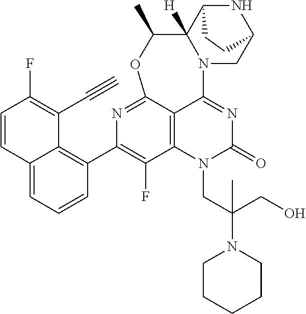 [SMILES: C#Cc1c(F)ccc2cccc(-c3nc4c5c(nc(=O)n(CC(C)(CO)N6CCCCC6)c5c3F)N3C[C@H]5CC[C@H](N5)[C@H]3[C@H](C)O4)c12] | (8S,8aS,9S,12R)-5-(8-ethynyl-7- fluoronaphthalen-1-yl)-4-fluoro- 3-(3-hydroxy-2-methyl-2- (piperidin-1-yl)propyl)-8- methyl-8a,9,10,11,12,13- hexahydro-8H-7-oxa- 1,3,6,13a,14-pentaaza-9,12- methanonaphtho[1,8- ab]heptalen-2(3H)-one | 641 [M + H]⁺ |
| 404 | 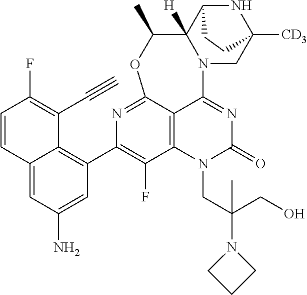 [SMILES: [2H]C([2H])([2H])[C@]12CC[C@H](N1)[C@H]1[C@H](C)Oc3nc(-c4cc(N)cc5ccc(F)c(C#C)c45)c(F)c4c3c(nc(=O)n4CC(C)(CO)N3CCC3)N1C2] | (8S,8aS,9S,12R)-5-(3-amino-8- ethynyl-7-fluoronaphthalen-1- yl)-3-(2-(azetidin-1-yl)-3- hydroxy-2-methylpropyl)-4- fluoro-8-methyl-12-(methyl-d3)- 8a,9,10,11,12,13-hexahydro-8H- 7-oxa-1,3,6,13a,14-pentaaza- 9,12-methanonaphtho[1,8- ab]heptalen-2(3H)-one | 645 [M + H]⁺ |
| 405 | 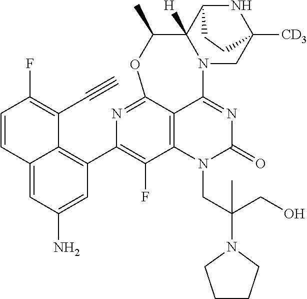 [SMILES: [2H]C([2H])([2H])[C@]12CC[C@H](N1)[C@H]1[C@H](C)Oc3nc(-c4cc(N)cc5ccc(F)c(C#C)c45)c(F)c4c3c(nc(=O)n4CC(C)(CO)N3CCCC3)N1C2] | (8S,8aS,9S,12R)-5-(3-amino-8- ethynyl-7-fluoronaphthalen-1- yl)-4-fluoro-3-(3-hydroxy-2- methyl-2-(pyrrolidin-1- yl)propyl)-8-methyl-12-(methyl- d3)-8a,9,10,11,12,13-hexahydro- 8H-7-oxa-1,3,6,13a,14- pentaaza-9,12- methanonaphtho[1,8- ab]heptalen-2(3H)-one | 659 [M + H]⁺ |
| 406 | 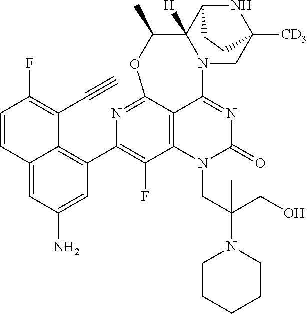 [SMILES: [2H]C([2H])([2H])[C@]12CC[C@H](N1)[C@H]1[C@H](C)Oc3nc(-c4cc(N)cc5ccc(F)c(C#C)c45)c(F)c4c3c(nc(=O)n4CC(C)(CO)N3CCCCC3)N1C2] | (8S,8aS,9S,12R)-5-(3-amino-8- ethynyl-7-fluoronaphthalen-1- yl)-4-fluoro-3-(3-hydroxy-2- methyl-2-(piperidin-1- yl)propyl)-8-methyl-12-(methyl- d3)-8a,9,10,11,12,13-hexahydro- 8H-7-oxa-1,3,6,13a,14- pentaaza-9,12- methanonaphtho[1,8- ab]heptalen-2(3H)-one | 673 [M + H]⁺ |
| 407 | 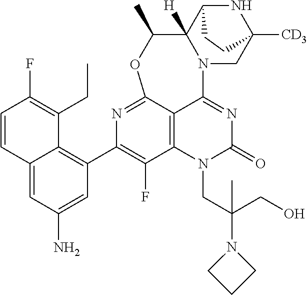 [SMILES: [2H]C([2H])([2H])[C@]12CC[C@H](N1)[C@H]1[C@H](C)Oc3nc(-c4cc(N)cc5ccc(F)c(CC)c45)c(F)c4c3c(nc(=O)n4CC(C)(CO)N3CCC3)N1C2] | (8S,8aS,9S,12R)-5-(3-amino-8- ethyl-7-fluoronaphthalen-1-yl)- 3-(2-(azetidin-1-yl)-3-hydroxy- 2-methylpropyl)-4-fluoro-8- methyl-12-(methyl-d3)- 8a,9,10,11,12,13-hexahydro-8H- 7-oxa-1,3,6,13a,14-pentaaza- 9,12-methanonaphtho[1,8- ab]heptalen-2(3H)-one | 649 [M + H]⁺ |
| 408 | 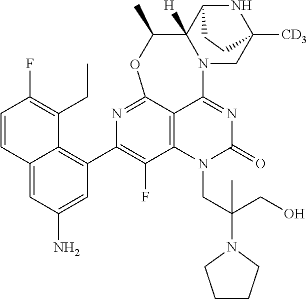 [SMILES: [2H]C([2H])([2H])[C@]12CC[C@H](N1)[C@H]1[C@H](C)Oc3nc(-c4cc(N)cc5ccc(F)c(CC)c45)c(F)c4c3c(nc(=O)n4CC(C)(CO)N3CCCC3)N1C2] | (8S,8aS,9S,12R)-5-(3-amino-8- ethyl-7-fluoronaphthalen-1-yl)- 4-fluoro-3-(3-hydroxy-2-methyl- 2-(pyrrolidin-1-yl)propyl)-8- methyl-12-(methyl-d3)- 8a,9,10,11,12,13-hexahydro-8H- 7-oxa-1,3,6,13a,14-pentaaza- 9,12-methanonaphtho[1,8- ab]heptalen-2(3H)-one | 663 [M + H]⁺ |
| 409 | 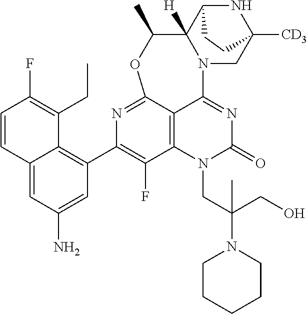 [SMILES: [2H]C([2H])([2H])[C@]12CC[C@H](N1)[C@H]1[C@H](C)Oc3nc(-c4cc(N)cc5ccc(F)c(CC)c45)c(F)c4c3c(nc(=O)n4CC(C)(CO)N3CCCCC3)N1C2] | (8S,8aS,9S,12R)-5-(3-amino-8- ethyl-7-fluoronaphthalen-1-yl)- 4-fluoro-3-(3-hydroxy-2-methyl- 2-(piperidin-1-yl)propyl)-8- methyl-12-(methyl-d3)- 8a,9,10,11,12,13-hexahydro-8H- 7-oxa-1,3,6,13a,14-pentaaza- 9,12-methanonaphtho[1,8- ab]heptalen-2(3H)-one | 677 [M + H]⁺ |
| 410 | 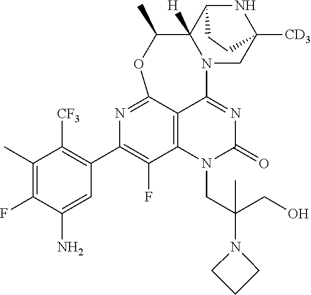 [SMILES: [2H]C([2H])([2H])[C@]12CC[C@H](N1)[C@H]1[C@H](C)Oc3nc(-c4cc(N)c(F)c(C)c4C(F)(F)F)c(F)c4c3c(nc(=O)n4CC(C)(CO)N3CCC3)N1C2] | (8S,8aS,9S,12R)-5-(5-amino-4- fluoro-3-methyl-2- (trifluoromethyl)phenyl)-3-(2- (azetidin-1-yl)-3-hydroxy-2- methylpropyl)-4-fluoro-8- methyl-12-(methyl-d3)- 8a,9,10,11,12,13-hexahydro-8H- 7-oxa-1,3,6,13a,14-pentaaza- 9,12-methanonaphtho[1,8- ab]heptalen-2(3H)-one | 653 [M + H]⁺ |
| 411 | 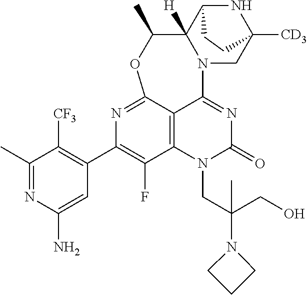 [SMILES: [2H]C([2H])([2H])[C@]12CC[C@H](N1)[C@H]1[C@H](C)Oc3nc(-c4cc(N)nc(C)c4C(F)(F)F)c(F)c4c3c(nc(=O)n4CC(C)(CO)N3CCC3)N1C2] | (8S,8aS,9S,12R)-5-(6-amino-2- methyl-3- (trifluoromethyl)pyridin-4-yl)-3- (2-(azetidin-1-yl)-3-hydroxy-2- methylpropyl)-4-fluoro-8- methyl-12-(methyl-d3)- 8a,9,10,11,12,13-hexahydro-8H- 7-oxa-1,3,6,13a,14-pentaaza- 9,12-methanonaphtho[1,8- ab]heptalen-2(3H)-one | 636 [M + H]⁺ |
| 412 | 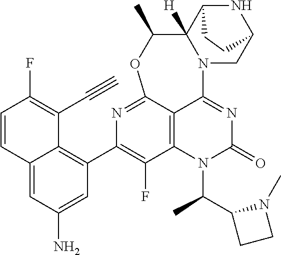 [SMILES: C#Cc1c(F)ccc2cc(N)cc(-c3nc4c5c(nc(=O)n([C@H](C)[C@H]6CCN6C)c5c3F)N3C[C@H]5CC[C@H](N5)[C@H]3[C@H](C)O4)c12] | (8S,8aS,9S,12R)-5-(3-amino-8- ethynyl-7-fluoronaphthalen-1- yl)-4-fluoro-8-methyl-3-((R)-1- ((R)-1-methylazetidin-2- yl)ethyl)-8a,9,10,11,12,13- hexahydro-8H-7-oxa- 1,3,6,13a,14-pentaaza-9,12- methanonaphtho[1,8- ab]heptalen-2(3H)-one | 598 [M + H]⁺ |
| 413 | 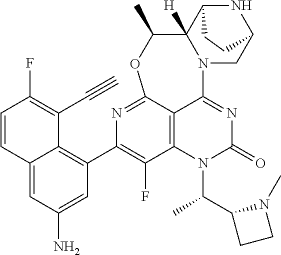 [SMILES: C#Cc1c(F)ccc2cc(N)cc(-c3nc4c5c(nc(=O)n([C@@H](C)[C@H]6CCN6C)c5c3F)N3C[C@H]5CC[C@H](N5)[C@H]3[C@H](C)O4)c12] | (8S,8aS,9S,12R)-5-(3-amino-8- ethynyl-7-fluoronaphthalen-1- yl)-4-fluoro-8-methyl-3-((S)-1- ((R)-1-methylazetidin-2- yl)ethyl)-8a,9,10,11,12,13- hexahydro-8H-7-oxa- 1,3,6,13a,14-pentaaza-9,12- methanonaphtho[1,8- ab]heptalen-2(3H)-one | 598 [M + H]⁺ |
| 414 | 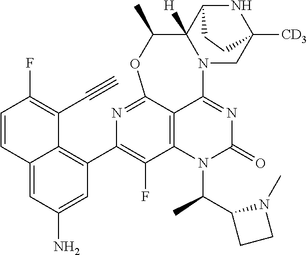 [SMILES: [2H]C([2H])([2H])[C@]12CC[C@H](N1)[C@H]1[C@H](C)Oc3nc(-c4cc(N)cc5ccc(F)c(C#C)c45)c(F)c4c3c(nc(=O)n4[C@H](C)[C@H]3CCN3C)N1C2] | (8S,8aS,9S,12R)-5-(3-amino-8- ethynyl-7-fluoronaphthalen-1- yl)-4-fluoro-8-methyl-12- (methyl-d3)-3-((R)-1-((R)-1- methylazetidin-2-yl)ethyl)- 8a,9,10,11,12,13-hexahydro-8H- 7-oxa-1,3,6,13a,14-pentaaza- 9,12-methanonaphtho[1,8- ab]heptalen-2(3H)-one | 615 [M + H]⁺ |
| 415 | 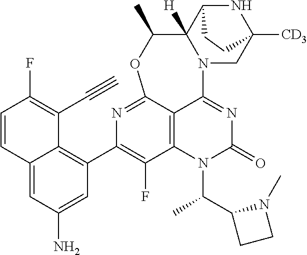 [SMILES: [2H]C([2H])([2H])[C@]12CC[C@H](N1)[C@H]1[C@H](C)Oc3nc(-c4cc(N)cc5ccc(F)c(C#C)c45)c(F)c4c3c(nc(=O)n4[C@@H](C)[C@H]3CCN3C)N1C2] | (8S,8aS,9S,12R)-5-(3-amino-8- ethynyl-7-fluoronaphthalen-1- yl)-4-fluoro-8-methyl-12- (methyl-d3)-3-((S)-1-((R)-1- methylazetidin-2-yl)ethyl)- 8a,9,10,11,12,13-hexahydro-8H- 7-oxa-1,3,6,13a,14-pentaaza- 9,12-methanonaphtho[1,8- ab]heptalen-2(3H)-one | 615 [M + H]⁺ |
| 416 | 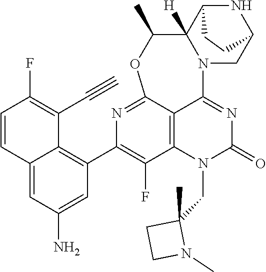 | (8S,8aS,9S,12R)-5-(3-amino-8- ethynyl-7-fluoronaphthalen-1- yl)-3-(((R)-1,2-dimethylazetidin- 2-yl)methyl)-4-fluoro-8-methyl- 8a,9,10,11,12,13-hexahydro-8H- 7-oxa-1,3,6,13a,14-pentaaza- 9,12-methanonaphtho[1,8- ab]heptalen-2(3H)-one | 598 [M + H]⁺ |
| 417 | 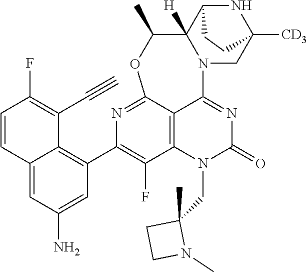 [SMILES: [2H]C([2H])([2H])[C@]12CC[C@H](N1)[C@H]1[C@H](C)Oc3nc(-c4cc(N)cc5ccc(F)c(C#C)c45)c(F)c4c3c(nc(=O)n4C[C@@]3(C)CCN3C)N1C2] | (8S,8aS,9S,12R)-5-(3-amino-8- ethynyl-7-fluoronaphthalen-1- yl)-3-(((R)-1,2-dimethylazetidin- 2-yl)methyl)-4-fluoro-8-methyl- 12-(methyl-d3)- 8a,9,10,11,12,13-hexahydro-8H- 7-oxa-1,3,6,13a,14-pentaaza- 9,12-methanonaphtho[1,8- ab]heptalen-2(3H)-one | 615 [M + H]⁺ |
| 418 | 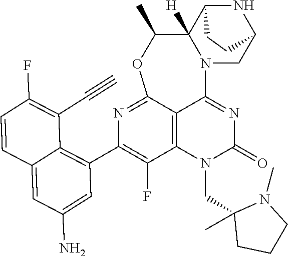 [SMILES: C#Cc1c(F)ccc2cc(N)cc(-c3nc4c5c(nc(=O)n(C[C@@]6(C)CCCN6C)c5c3F)N3C[C@H]5CC[C@H](N5)[C@H]3[C@H](C)O4)c12] | (8S,8aS,9S,12R)-5-(3-amino-8- ethynyl-7-fluoronaphthalen-1- yl)-3-(((R)-1,2- dimethylpyrrolidin-2- yl)methyl)-4-fluoro-8-methyl- 8a,9,10,11,12,13-hexahydro-8H- 7-oxa-1,3,6,13a,14-pentaaza- 9,12-methanonaphtho[1,8- ab]heptalen-2(3H)-one | 612 [M + H]⁺ |
| 419 | 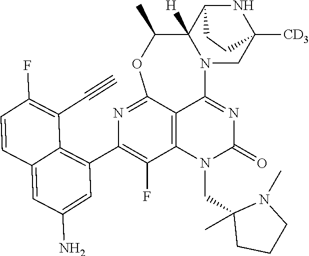 [SMILES: [2H]C([2H])([2H])[C@]12CC[C@H](N1)[C@H]1[C@H](C)Oc3nc(-c4cc(N)cc5ccc(F)c(C#C)c45)c(F)c4c3c(nc(=O)n4C[C@@]3(C)CCCN3C)N1C2] | (8S,8aS,9S,12R)-5-(3-amino-8- ethynyl-7-fluoronaphthalen-1- yl)-3-(((R)-1,2- dimethylpyrrolidin-2- yl)methyl)-4-fluoro-8-methyl- 12-(methyl-d3)- 8a,9,10,11,12,13-hexahydro-8H- 7-oxa-1,3,6,13a,14-pentaaza- 9,12-methanonaphtho[1,8- ab]heptalen-2(3H)-one | 629 [M + H]⁺ |
| 420 | 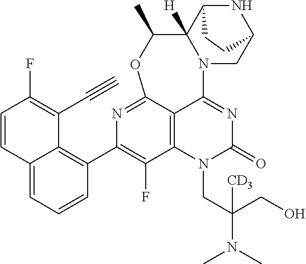 [SMILES: [2H]C([2H])([2H])C(CO)(Cn1c(=O)nc2c3c(nc(-c4cccc5ccc(F)c(C#C)c45)c(F)c31)O[C@@H](C)[C@@H]1[C@@H]3CC[C@H](CN21)N3)N(C)C] | (8S,8aS,9S,12R)-3-(2- (dimethylamino)-2- (hydroxymethyl)propyl-3,3,3- d3)-5-(8-ethynyl-7- fluoronaphthalen-1-yl)-4-fluoro- 8-methyl-8a,9,10,11,12,13- hexahydro-8H-7-oxa- 1,3,6,13a,14-pentaaza-9,12- methanonaphtho[1,8- ab]heptalen-2(3H)-one | 604 [M + H]⁺ |
| 421 | 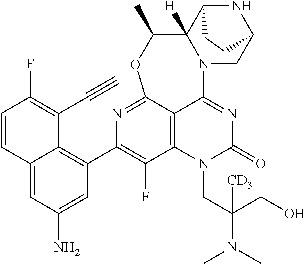 [SMILES: [2H]C([2H])([2H])C(CO)(Cn1c(=O)nc2c3c(nc(-c4cc(N)cc5ccc(F)c(C#C)c45)c(F)c31)O[C@@H](C)[C@@H]1[C@@H]3CC[C@H](CN21)N3)N(C)C] | (8S,8aS,9S,12R)-5-(3-amino-8- ethynyl-7-fluoronaphthalen-1- yl)-3-(2-(dimethylamino)-2- (hydroxymethyl)propyl-3,3,3- d3)-4-fluoro-8-methyl- 8a,9,10,11,12,13-hexahydro-8H- 7-oxa-1,3,6,13a,14-pentaaza- 9,12-methanonaphtho[1,8- ab]heptalen-2(3H)-one | 619 [M + H]⁺ |
| 422 | 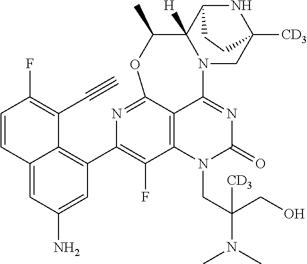 [SMILES: [2H]C([2H])([2H])C(CO)(Cn1c(=O)nc2c3c(nc(-c4cc(N)cc5ccc(F)c(C#C)c45)c(F)c31)O[C@@H](C)[C@@H]1[C@@H]3CC[C@](C([2H])([2H])[2H])(CN21)N3)N(C)C] | (8S,8aS,9S,12R)-5-(3-amino-8- ethynyl-7-fluoronaphthalen-1- yl)-3-(2-(dimethylamino)-2- (hydroxymethyl)propyl-3,3,3- d3)-4-fluoro-8-methyl-12- (methyl-d3)-8a,9,10,11,12,13- hexahydro-8H-7-oxa- 1,3,6,13a,14-pentaaza-9,12- methanonaphtho[1,8- ab]heptalen-2(3H)-one | 636 [M + H]⁺ |
| 423 | 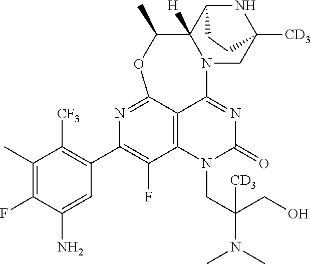 [SMILES: [2H]C([2H])([2H])C(CO)(Cn1c(=O)nc2c3c(nc(-c4cc(N)c(F)c(C)c4C(F)(F)F)c(F)c31)O[C@@H](C)[C@@H]1[C@@H]3CC[C@](C([2H])([2H])[2H])(CN21)N3)N(C)C] | (8S,8aS,9S,12R)-5-(5-amino-4- fluoro-3-methyl-2- (trifluoromethyl)phenyl)-3-(2- (dimethylamino)-2- (hydroxymethyl)propyl-3,3,3- d3)-4-fluoro-8-methyl-12- (methyl-d3)-8a,9,10,11,12,13- hexahydro-8H-7-oxa- 1,3,6,13a,14-pentaaza-9,12- methanonaphtho[1,8- ab]heptalen-2(3H)-one | 644 [M + H]⁺ |
| 424 | 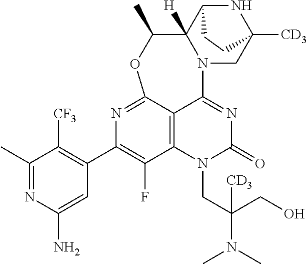 [SMILES: [2H]C([2H])([2H])C(CO)(Cn1c(=O)nc2c3c(nc(-c4cc(N)nc(C)c4C(F)(F)F)c(F)c31)O[C@@H](C)[C@@H]1[C@@H]3CC[C@](C([2H])([2H])[2H])(CN21)N3)N(C)C] | (8S,8aS,9S,12R)-5-(6-amino-2- methyl-3- (trifluoromethyl)pyridin-4-yl)-3- (2-(dimethylamino)-2- (hydroxymethyl)propyl-3,3,3- d3)-4-fluoro-8-methyl-12- (methyl-d3)-8a,9,10,11,12,13- hexahydro-8H-7-oxa- 1,3,6,13a,14-pentaaza-9,12- methanonaphtho[1,8- ab]heptalen-2(3H)-one | 627 [M + H]⁺ |
| 425 | 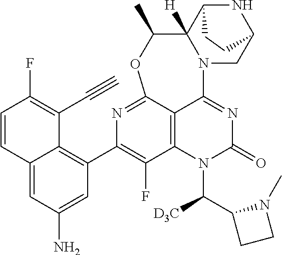 [SMILES: [2H]C([2H])([2H])[C@H]([C@H]1CCN1C)n1c(=O)nc2c3c(nc(-c4cc(N)cc5ccc(F)c(C#C)c45)c(F)c31)O[C@@H](C)[C@@H]1[C@@H]3CC[C@H](CN21)N3] | (8S,8aS,9S,12R)-5-(3-amino-8- ethynyl-7-fluoronaphthalen-1- yl)-4-fluoro-8-methyl-3-((R)-1- ((R)-1-methylazetidin-2- yl)ethyl-2,2,2-d3)- 8a,9,10,11,12,13-hexahydro-8H- 7-oxa-1,3,6,13a,14-pentaaza- 9,12-methanonaphtho[1,8- ab]heptalen-2(3H)-one | 601 [M + H]⁺ |
| 426 | 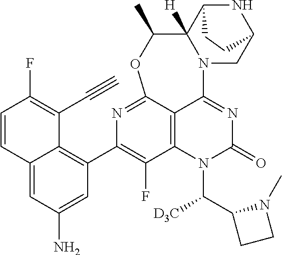 [SMILES: [2H]C([2H])([2H])[C@@H]([C@H]1CCN1C)n1c(=O)nc2c3c(nc(-c4cc(N)cc5ccc(F)c(C#C)c45)c(F)c31)O[C@@H](C)[C@@H]1[C@@H]3CC[C@H](CN21)N3] | (8S,8aS,9S,12R)-5-(3-amino-8- ethynyl-7-fluoronaphthalen-1- yl)-4-fluoro-8-methyl-3-(((R)-1- methyl-2-(methyl-d3)azetidin-2- yl)methyl)-8a,9,10,11,12,13- hexahydro-8H-7-oxa- 1,3,6,13a,14-pentaaza-9,12- methanonaphtho[1,8- ab]heptalen-2(3H)-one | 601 [M + H]⁺ |
| 427 | 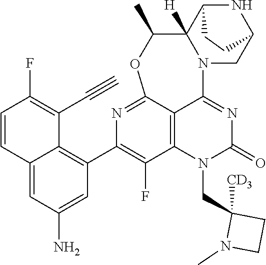 [SMILES: [2H]C([2H])([2H])[C@]1(Cn2c(=O)nc3c4c(nc(-c5cc(N)cc6ccc(F)c(C#C)c56)c(F)c42)O[C@@H](C)[C@@H]2[C@@H]4CC[C@H](CN32)N4)CCN1C] | (8S,8aS,9S,12R)-5-(3-amino-8- ethynyl-7-fluoronaphthalen-1- yl)-4-fluoro-8-methyl-3-((S)-1- ((R)-1-methylazetidin-2- yl)ethyl-2,2,2-d3)- 8a,9,10,11,12,13-hexahydro-8H- 7-oxa-1,3,6,13a,14-pentaaza- 9,12-methanonaphtho[1,8- ab]heptalen-2(3H)-one | 601 [M + H]⁺ |
| 428 | 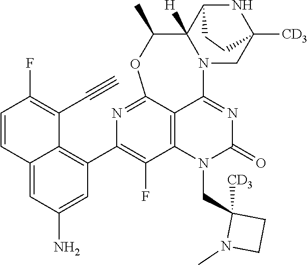 [SMILES: [2H]C([2H])([2H])[C@]12CC[C@H](N1)[C@H]1[C@H](C)Oc3nc(-c4cc(N)cc5ccc(F)c(C#C)c45)c(F)c4c3c(nc(=O)n4C[C@@]3(C([2H])([2H])[2H])CCN3C)N1C2] | (8S,8aS,9S,12R)-5-(3-amino-8- ethynyl-7-fluoronaphthalen-1- yl)-4-fluoro-8-methyl-12- (methyl-d3)-3-(((R)-1-methyl-2- (methyl-d3)azetidin-2- yl)methyl)-8a,9,10,11,12,13- hexahydro-8H-7-oxa- 1,3,6,13a,14-pentaaza-9,12- methanonaphtho[1,8- ab]heptalen-2(3H)-one | 618 [M + H]⁺ |
| 429 | 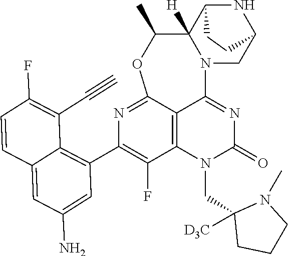 [SMILES: [2H]C([2H])([2H])[C@]1(Cn2c(=O)nc3c4c(nc(-c5cc(N)cc6ccc(F)c(C#C)c56)c(F)c42)O[C@@H](C)[C@@H]2[C@@H]4CC[C@H](CN32)N4)CCCN1C] | (8S,8aS,9S,12R)-5-(3-amino-8- ethynyl-7-fluoronaphthalen-1- yl)-4-fluoro-8-methyl-3-(((R)-1- methyl-2-(methyl-d3)pyrrolidin- 2-yl)methyl)-8a,9,10,11,12,13- hexahydro-8H-7-oxa- 1,3,6,13a,14-pentaaza-9,12- methanonaphtho[1,8- ab]heptalen-2(3H)-one | 615 [M + H]⁺ |
| 430 | 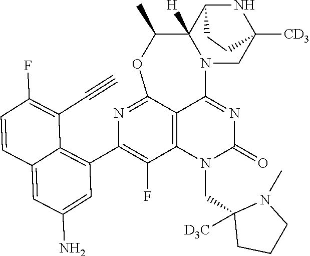 [SMILES: [2H]C([2H])([2H])[C@]12CC[C@H](N1)[C@H]1[C@H](C)Oc3nc(-c4cc(N)cc5ccc(F)c(C#C)c45)c(F)c4c3c(nc(=O)n4C[C@@]3(C([2H])([2H])[2H])CCCN3C)N1C2] | (8S,8aS,9S,12R)-5-(3-amino-8- ethynyl-7-fluoronaphthalen-1- yl)-4-fluoro-8-methyl-12- (methyl-d3)-3-(((R)-1-methyl-2- (methyl-d3)pyrrolidin-2- yl)methyl)-8a,9,10,11,12,13- hexahydro-8H-7-oxa- 1,3,6,13a,14-pentaaza-9,12- methanonaphtho[1,8- ab]heptalen-2(3H)-one | 632 [M + H]⁺ |

### Example 2

¹H NMR: (400 MHz, CD₃OD) δ 8.13-8.05 (m, 2H), 7.67-7.55 (m, 2H), 7.47-7.38 (m, 1H), 5.02-4.93 (m, 1H), 4.92-4.86 (m, 2H), 4.64-4.40 (m, 2H), 4.15-4.08 (m, 1H), 3.78-3.66 (m, 2H), 3.66-3.58 (m, 2H), 3.58-3.46 (m, 2H), 3.45-3.34 (m, 2H), 3.29-3.18 (m, 1H), 3.09-2.93 (m, 3H), 2.86-2.75 (m, 1H), 2.53-2.38 (m, 1H), 2.24-2.11 (m, 1H), 2.00-1.79 (m, 4H), 1.59-1.50 (m, 1H).

### Example 3

¹H NMR (400 MHz, CD₃OD) δ 8.13-8.04 (m, 2H), 7.67-7.55 (m, 2H), 7.48-7.38 (m, 1H), 5.03-4.91 (m, 1H), 4.78 (s, 4H), 4.65-4.41 (m, 2H), 4.13 (s, 1H), 3.76 (s, 1H), 3.71-3.58 (m, 4H), 3.48 (d, 6H), 3.13 (s, 1H), 2.94 (d, 2H), 2.02-1.76 (m, 4H).

### Example 24

¹H NMR: (400 MHz, CD₃OD) δ 7.86-7.80 (m, 1H), 7.35-7.27 (m, 2H), 7.23-7.13 (m, 1H), 5.09-4.94 (m, 1H), 4.94-4.87 (m, 2H), 4.62-4.41 (m, 2H), 4.15-4.07 (m, 1H), 3.80-3.72 (m, 1H), 3.70-3.62 (m, 2H), 3.62-3.44 (m, 3H), 3.44-3.33 (m, 2H), 3.29-3.16 (m, 1H), 3.14-2.91 (m, 3H), 2.86-2.76 (m, 1H), 2.55-2.36 (m, 1H), 2.23-2.09 (m, 1H), 1.98-1.78 (m, 4H), 1.59-1.50 (m, 1H).

### Example 46a

¹H NMR: (400 MHz, CD₃OD) δ 7.71-7.62 (m, 1H), 7.32-7.19 (m, 2H), 7.11-6.98 (m, 1H), 5.18-5.02 (m, 3H), 4.72-4.50 (m, 2H), 4.37-4.22 (m, 1H), 4.12-3.83 (m, 4H), 3.77-3.62 (m, 3H), 3.46-3.34 (m, 2H), 3.29-3.16 (m, 1H), 3.14-3.05 (m, 1H), 2.96-2.85 (m, 1H), 2.60-2.17 (m, 4H), 2.15-1.93 (m, 4H), 1.86-1.73 (m, 1H), 0.95-0.80 (m, 3H).

### Example 46b

¹H NMR: (400 MHz, CD₃OD) δ 7.69-7.62 (m, 1H), 7.30-7.20 (m, 2H), 7.05 (m, 1H), 5.14-4.96 (m, 3H), 4.71-4.49 (m, 2H), 4.31-4.20 (m, 1H), 4.06-3.93 (m, 2H), 3.92-3.76 (m, 2H), 3.70-3.56 (m, 3H), 3.43-3.34 (m, 1H), 3.29-3.12 (m, 2H), 3.12-3.03 (m, 1H), 2.93-2.84 (m, 1H), 2.57-2.16 (m, 4H), 2.14-1.88 (m, 4H), 1.78-1.69 (m, 1H), 0.95-0.79 (m, 3H).

### Example 47

¹H NMR (400 MHz, CD₃OD) δ 7.68-7.62 (m, 1H), 7.29-7.19 (m, 2H), 7.12-6.98 (m, 1H), 5.03-4.89 (m, 1H), 4.77 (s, 4H), 4.62-4.43 (m, 2H), 4.19-4.08 (m, 1H), 3.75 (s, 1H), 3.64 (d, 3H), 3.48 (d, 6H), 3.13 (s, 1H), 2.94 (d, 2H), 2.62-2.16 (m, 2H), 2.00-1.75 (m, 4H), 0.96-0.79 (m, 3H).

### Example 57

¹H NMR: (400 MHz, CD₃OD) δ 6.59 (s, 1H), 4.96-4.88 (m, 3H), 4.59-4.53 (m, 1H), 4.47-4.39 (m, 1H), 4.12-4.06 (m, 1H), 3.75-3.64 (m, 2H), 3.63-3.53 (m, 2H), 3.53-3.40 (m, 2H), 3.40-3.35 (m, 1H), 3.25-3.19 (m, 1H), 3.06-2.94 (m, 3H), 2.86-2.78 (m, 1H), 2.50-2.44 (m, 1H), 2.43 (s, 3H), 2.20-2.09 (m, 1H), 1.96-1.78 (m, 4H), 1.60-1.50 (m, 1H).

### Example 200a

¹H NMR: (400 MHz, CD₃OD) δ 8.12-8.04 (m, 2H), 7.67-7.53 (m, 2H), 7.47-7.38 (m, 1H), 5.41-5.24 (m, 1H), 5.02-4.87 (m, 2H), 4.84-4.82 (m, 1H), 4.61-4.51 (m, 1H), 4.07 (d, J=8.3 Hz, 1H), 3.83-3.65 (m, 2H), 3.64-3.56 (m, 2H), 3.54-3.41 (m, 2H), 3.39-3.33 (m, 1H), 3.26-3.17 (m, 1H), 3.14-2.93 (m, 3H), 2.86-2.73 (m, 1H), 2.53-2.37 (m, 1H), 2.23-2.02 (m, 2H), 1.93-1.75 (m, 3H), 1.61-1.49 (m, 4H).

### Example 200b

¹H NMR: (400 MHz, CD₃OD) δ 8.14-8.05 (m, 2H), 7.70-7.53 (m, 2H), 7.48-7.38 (m, 1H), 5.38-5.28 (m, 1H), 4.99-4.91 (m, 2H), 4.64-4.55 (m, 1H), 4.16-4.08 (m, 1H), 3.86-3.62 (m, 4H), 3.60-3.44 (m, 4H), 3.27-3.21 (m, 1H), 3.18-3.02 (m, 3H), 2.89-2.77 (m, 1H), 2.54-2.37 (m, 1H), 2.26-2.02 (m, 2H), 1.98-1.80 (m, 3H), 1.67-1.61 (m, 1H), 1.60-1.53 (m, 3H).

### Example 202a

¹H NMR: (400 MHz, CD₃OD) δ 8.12-8.05 (m, 2H), 7.69-7.54 (m, 2H), 7.47-7.38 (m, 1H), 5.53-5.32 (m, 1H), 5.31-5.22 (m, 1H), 4.97 (d, J=20.4 Hz, 2H), 4.61-4.52 (m, 1H), 4.10-4.04 (m, 1H), 3.82-3.50 (m, 4H), 3.50-3.37 (m, 4H), 3.29-3.25 (m, 1H), 3.24-3.12 (m, 2H), 3.03-2.95 (m, 1H), 2.89-2.78 (m, 1H), 2.36-2.18 (m, 1H), 2.07-1.97 (m, 1H), 1.91-1.74 (m, 3H), 1.61-1.52 (m, 3H).

### Example 202b

¹H NMR: (400 MHz, CD₃OD) δ 8.13-8.04 (m, 2H), 7.68-7.54 (m, 2H), 7.47-7.38 (m, 1H), 5.32-5.24 (m, 1H), 4.84-4.70 (m, 3H), 4.61-4.52 (m, 1H), 4.07 (d, J=8.4 Hz, 1H), 3.83-3.54 (m, 4H), 3.53-3.40 (m, 2H), 3.29-3.11 (m, 4H), 3.10-2.96 (m, 2H), 2.95-2.83 (m, 1H), 2.51-2.29 (m, 1H), 2.11-2.00 (m, 1H), 1.96-1.77 (m, 3H), 1.62-1.53 (m, 3H).

### Example 203a

¹H NMR: (400 MHz, CD₃OD) δ 8.15-8.02 (m, 2H), 7.70-7.50 (m, 2H), 7.49-7.36 (m, 1H), 6.54 (d, J=85.4 Hz, 1H), 5.31-5.21 (m, 1H), 4.60-4.50 (m, 1H), 4.11-4.03 (m, 1H), 3.95-3.87 (m, 1H), 3.81-3.58 (m, 3H), 3.50-3.41 (m, 2H), 3.38-3.35 (m, 2H), 3.25-3.17 (m, 1H), 3.11-2.94 (m, 2H), 2.90-2.76 (m, 2H), 2.48-2.31 (m, 1H), 2.25-2.12 (m, 1H), 2.07-1.96 (m, 1H), 1.93-1.76 (m, 3H), 1.67-1.49 (m, 4H).

### Example 203b

¹H NMR: (400 MHz, CD₃OD) δ 8.12-8.05 (m, 2H), 7.67-7.53 (m, 2H), 7.47-7.38 (m, 1H), 6.50 (d, J=84.0 Hz, 1H), 5.32-5.22 (m, 1H), 4.61-4.51 (m, 1H), 4.10-4.04 (m, 1H), 3.94-3.87 (m, 1H), 3.80-3.50 (m, 3H), 3.48-3.34 (m, 4H), 3.24-3.16 (m, 1H), 3.10-2.94 (m, 2H), 2.89-2.76 (m, 2H), 2.48-2.33 (m, 1H), 2.23-2.09 (m, 1H), 2.08-1.98 (m, 1H), 1.93-1.77 (m, 3H), 1.63-1.53 (m, 4H).

### Example 222

¹H NMR: (400 MHz, CD₃OD) δ 7.90-7.78 (m, 1H), 7.38-7.27 (m, 2H), 7.26-7.07 (m, 1H), 5.33-5.24 (m, 1H), 4.95-4.89 (m, 2H), 4.61-4.50 (m, 1H), 4.12-4.03 (m, 1H), 3.73-3.70 (m, 1H), 3.69-3.64 (m, 1H), 3.62-3.60 (m, 1H), 3.58-3.46 (m, 2H), 3.45-3.38 (m, 2H), 3.36-3.34 (m, 1H), 3.25-3.18 (m, 1H), 3.14-2.95 (m, 3H), 2.86-2.76 (m, 1H), 2.53-2.38 (m, 1H), 2.21-2.01 (m, 2H), 1.96-1.77 (m, 3H), 1.63-1.49 (m, 4H).

### Example 227

¹H NMR: (400 MHz, CD₃OD) δ 7.70-7.60 (m, 1H), 7.32-7.18 (m, 2H), 7.13-6.93 (m, 1H), 5.56-5.22 (m, 2H), 4.63-4.50 (m, 1H), 4.41-4.22 (m, 2H), 4.17-4.04 (m, 1H), 3.74-3.64 (m, 1H), 3.62-3.46 (m, 1H), 3.41-3.35 (m, 1H), 3.27-3.12 (m, 2H), 3.10-3.02 (m, 1H), 2.56-2.41 (m, 1H), 2.39-2.20 (m, 2H), 2.20-2.13 (m, 1H), 2.07-2.01 (m, 2H), 1.91 (d, J=14.6 Hz, 2H), 1.84-1.76 (m, 2H), 1.56 (d, J=6.1 Hz, 3H), 1.36-1.25 (m, 3H), 1.00-0.74 (m, 3H).

### Example 228

¹H NMR: (400 MHz, CD₃OD) δ 7.68-7.62 (m, 1H), 7.30-7.19 (m, 2H), 7.13-6.94 (m, 1H), 5.33-5.24 (m, 1H), 4.94-4.90 (m, 1H), 4.83-4.81 (m, 1H), 4.63-4.53 (m, 1H), 4.11-4.04 (m, 1H), 3.73-3.57 (m, 4H), 3.53-3.38 (m, 2H), 3.34-3.32 (m, 1H), 3.26-3.16 (m, 1H), 3.14-2.92 (m, 3H), 2.86-2.78 (m, 1H), 2.55-2.40 (m, 2H), 2.24-1.95 (m, 3H), 1.94-1.74 (m, 3H), 1.60-1.49 (m, 4H), 1.00-0.77 (m, 3H).

### Example 309

¹H NMR: (400 MHz, CD₃OD) δ 7.40-7.35 (m, 1H), 7.04-6.99 (m, 1H), 4.99-4.94 (m, 1H), 4.93-4.87 (m, 2H), 4.61-4.55 (m, 1H), 4.48-4.40 (m, 1H), 4.14-4.09 (m, 1H), 3.76-3.64 (m, 2H), 3.63-3.53 (m, 2H), 3.52-3.39 (m, 2H), 3.38-3.33 (m, 1H), 3.27-3.21 (m, 1H), 3.08-2.95 (m, 3H), 2.87-2.78 (m, 1H), 2.52-2.39 (m, 1H), 2.21-2.09 (m, 1H), 1.99-1.75 (m, 4H), 1.59-1.50 (m, 1H).

### Example 320

¹H NMR (400 MHz, CD₃OD) δ 8.17-8.03 (m, 2H), 7.69-7.53 (m, 2H), 7.46 (m, 1H), 5.44-5.33 (m, 1H), 4.68-4.38 (m, 3H), 4.13 (m, 1H), 3.93 (d, J=120.2 Hz, 1H), 3.67 (s, 1H), 3.57 (s, 1H), 3.13 (m, 1H), 2.37 (d, J=3.5 Hz, 6H), 2.20 (d, J=7.4 Hz, 1H), 1.83-1.75 (m, 2H), 1.57 (m, 3H), 1.33 (s, 2H), 0.57 (m, 3H).

### Example 322

¹H NMR (400 MHz, CD₃OD) δ 8.15-8.06 (m, 2H), 7.67-7.53 (m, 2H), 7.45 (q, J=8.8 Hz, 1H), 5.54-5.34 (m, 1H), 4.58 (d, J=4.0 Hz, 2H), 4.49-4.40 (m, 1H), 4.15 (dd, J=9.7, 4.8 Hz, 1H), 3.67 (d, J=2.0 Hz, 1H), 3.57 (d, J=5.4 Hz, 1H), 3.21-3.09 (m, 2H), 2.28 (s, 6H), 2.21-2.10 (m, 3H), 2.03 (d, J=8.4 Hz, 2H), 1.88-1.68 (m, 5H), 1.58 (dd, J=8.5, 6.3 Hz, 3H).

### Example 323

¹H NMR (400 MHz, CD₃OD) δ 8.09 (dd, J=8.6, 6.1 Hz, 2H), 7.67-7.53 (m, 2H), 7.44 (q, J=8.8 Hz, 1H), 5.48 (s, 1H), 4.56 (d, J=13.2 Hz, 1H), 4.39 (s, 1H), 4.13 (d, J=10.0 Hz, 1H), 4.02-3.32 (m, 4H), 3.12 (dd, J=23.4, 13.2 Hz, 1H), 2.71 (d, J=33.7 Hz, 4H), 2.12 (d, J=7.4 Hz, 4H), 1.95 (s, 1H), 1.87-1.59 (m, 9H), 1.58 (dd, J=6.1, 3.0 Hz, 3H).

### Example 325a

¹H NMR (400 MHz, CD₃OD) δ 8.16-8.09 (m, 2H), 7.73-7.62 (m, 2H), 7.45 (t, J=8.9 Hz, 1H), 5.52 (dd, J=13.2, 2.7 Hz, 1H), 5.05 (s, 1H), 4.13 (dd, J=20.0, 8.1 Hz, 2H), 3.69 (d, J=6.8 Hz, 2H), 3.60 (s, 1H), 3.48 (s, 2H), 3.23 (dd, J=14.6, 7.8 Hz, 2H), 3.10 (d, J=13.6 Hz, 1H), 2.57 (d, J=11.9 Hz, 3H), 2.12 (t, J=8.8 Hz, 1H), 1.94 (d, J=9.4 Hz, 2H), 1.82 (s, 2H), 1.60 (d, J=6.0 Hz, 3H), 1.54 (d, J=9.5 Hz, 1H), 1.47 (d, J=12.8 Hz, 1H), 1.40 (d, J=6.8 Hz, 3H), 1.16 (d, J=9.8 Hz, 1H), 0.60 (d, J=9.9 Hz, 1H), 0.42-0.27 (m, 2H).

### Example 325b

¹H NMR (400 MHz, CD₃OD) δ 8.13 (dd, J=8.8, 5.7 Hz, 2H), 7.70-7.58 (m, 2H), 7.47 (t, J=9.0 Hz, 1H), 5.40-5.09 (m, 2H), 4.57 (d, J=6.2 Hz, 1H), 4.12 (d, J=8.3 Hz, 1H), 3.93 (d, J=39.5 Hz, 1H), 3.79 (d, J=24.8 Hz, 2H), 3.66 (d, J=19.0 Hz, 1H), 3.38 (s, 2H), 3.21 (d, J=13.8 Hz, 2H), 2.57-2.31 (m, 4H), 2.19 (s, 1H), 1.88 (s, 4H), 1.56 (d, J=6.4 Hz, 3H), 1.45 (s, 1H), 1.39 (d, J=6.7 Hz, 3H), 1.07 (dd, J=9.0, 4.6 Hz, 1H), 0.61 (dd, J=12.0, 5.8 Hz, 1H), 0.36-0.24 (m, 2H).

### Example 326a

¹H NMR (400 MHz, CD₃OD) δ 8.04-7.97 (m, 2H), 7.58-7.43 (m, 2H), 7.35 (q, J=9.2 Hz, 1H), 5.27 (d, J=13.2 Hz, 1H), 4.48 (d, J=15.2 Hz, 2H), 4.42-4.34 (m, 1H), 4.00 (d, J=9.5 Hz, 1H), 3.77 (d, J=110.3 Hz, 1H), 3.53 (s, 1H), 3.43 (s, 1H), 3.32 (dd, J=8.3, 4.7 Hz, 1H), 3.13-2.96 (m, 3H), 2.28-1.93 (m, 3H), 1.78-1.58 (m, 5H), 1.53 (s, 2H), 1.47 (dd, J=6.2, 2.4 Hz, 5H), 0.57-0.14 (m, 4H).

### Example 326b

¹H NMR (400 MHz, CD₃OD) δ 8.16-8.06 (m, 2H), 7.67-7.52 (m, 2H), 7.45 (q, J=8.9 Hz, 1H), 5.41-5.32 (m, 1H), 4.64-4.43 (m, 3H), 4.10 (d, J=9.4 Hz, 1H), 3.90 (d, J=104.4 Hz, 1H), 3.63 (s, 1H), 3.54 (s, 1H), 3.47 (s, 2H), 3.28-3.23 (m, 1H), 3.16-3.08 (m, 1H), 2.44 (s, 1H), 2.28-2.16 (m, 2H), 2.03 (s, 1H), 1.90-1.70 (m, 6H), 1.64 (s, 2H), 1.56 (dd, J=6.1, 5.0 Hz, 3H), 0.77-0.64 (m, 1H), 0.47 (s, 2H), 0.26 (s, 1H).

### Example 328

¹H NMR: (400 MHz, CD₃OD) δ 7.68-7.63 (m, 1H), 7.27 (m, 1H), 7.23 (m, 1H), 7.07 (m, 1H), 6.43 (d, J=8.6 Hz, 1H), 4.75 (s, 4H), 4.68-4.54 (m, 3H), 3.96-3.84 (m, 2H), 3.84-3.70 (m, 2H), 3.64 (d, J=14.6 Hz, 2H), 3.55-3.45 (m, 8H), 2.98 (d, J=13.9 Hz, 2H), 2.57-2.44 (m, 1H), 2.18 (dd, J=12.1, 5.0 Hz, 1H), 1.40 (dd, J=21.9, 6.7 Hz, 3H), 0.91 (t, J=7.2 Hz, 2H), 0.80 (t, J=7.4 Hz, 2H).

### Example 329

¹H NMR: (400 MHz, CD₃OD) δ 7.61 (m, 1H), 7.22 (s, 1H), 7.20-7.13 (m, 1H), 7.03 (m, 1H), 6.21 (d, J=4.1 Hz, 1H), 4.68 (s, 4H), 4.48 (t, J=14.2 Hz, 2H), 3.68 (s, 1H), 3.57 (d, J=14.1 Hz, 4H), 3.44 (d, J=14.2 Hz, 6H), 2.93 (s, 2H), 2.25 (dd, J=96.2, 53.9 Hz, 4H), 2.00-1.69 (m, 4H), 0.86-0.78 (m, 3H).

### Example 330

¹H NMR: (400 MHz, CD₃OD) δ 7.84 (m, 1H), 7.31 (m, 2H), 6.42 (s, 1H), 5.10 (t, J=75.4 Hz, 2H), 4.64 (s, 1H), 3.91 (d, J=32.1 Hz, 3H), 3.80 (t, J=12.7 Hz, 2H), 3.70 (s, 1H), 3.67-3.51 (m, 4H), 3.43-3.27 (m, 1H), 3.15 (d, J=15.7 Hz, 3H), 2.86 (s, 1H), 2.46 (s, 1H), 2.35-2.15 (m, 3H), 2.12-1.91 (m, 4H), 1.42 (dd, J=14.7, 6.7 Hz, 3H).

### Example 331

¹H NMR: (400 MHz, CD₃OD) δ 7.85 (m, 1H), 7.35-7.29 (m, 2H), 7.11 (m, 1H), 6.20 (s, 1H), 4.96 (s, 1H), 4.62-4.50 (m, 2H), 4.44 (dd, J=13.6, 5.0 Hz, 1H), 3.85 (d, J=5.2 Hz, 1H), 3.75 (d, J=5.3 Hz, 1H), 3.51-3.47 (m, 2H), 3.40 (s, 1H), 3.18 (dd, J=19.7, 7.9 Hz, 3H), 3.12-3.02 (m, 2H), 2.98-2.92 (m, 2H), 2.77 (t, J=9.7 Hz, 2H), 2.56 (dd, J=22.4, 15.9 Hz, 2H), 2.07-1.92 (m, 4H), 1.61 (s, 2H).

### Example 332

¹H NMR: (400 MHz, CD₃OD) 7.85 (m, 1H), 7.33 (m, 2H), 7.15 (m, 1H), 6.22 (s, 1H), 4.62-4.52 (m, 2H), 4.40 (dd, J=13.7, 4.8 Hz, 1H), 3.95-3.85 (m, 2H), 3.55-3.51 (m, 2H), 3.28-2.93 (m, 9H), 2.83-2.76 (m, 2H), 2.71-2.54 (m, 2H), 2.08-1.97 (m, 4H), 1.61 (s, 2H).

### Example 339

¹H NMR (400 MHz, CD₃OD) δ 8.17-8.06 (m, 2H), 7.75-7.38 (m, 3H), 5.59-5.49 (m, 1H), 4.63-4.51 (m, 1H), 4.42-4.30 (m, 1H), 4.14 (d, J=10.2 Hz, 1H), 3.78 (d, J=98.9 Hz, 2H), 3.61-3.54 (m, 4H), 3.53-3.47 (m, 1H), 3.44-3.36 (m, 1H), 3.07 (t, J=12.1 Hz, 1H), 2.53-2.30 (m, 5H), 2.19-2.08 (m, 1H), 1.87-1.65 (m, 3H), 1.63-1.53 (m, 6H).

### Example 340

¹H NMR (400 MHz, CD₃OD) δ 8.15-8.08 (m, 2H), 7.69-7.56 (m, 2H), 7.51-7.42 (m, 1H), 5.29-5.31 (m, 1H), 4.80 (s, 1H), 4.57-4.47 (m, 1H), 4.10 (d, J=9.0 Hz, 1H), 3.89 (d, J=117.1 Hz, 1H), 3.66 (s, 1H), 3.61-3.46 (m, 5H), 3.20-2.98 (m, 2H), 2.91-2.74 (m, 1H), 2.46-2.34 (m, 4H), 2.23-2.15 (m, 1H), 1.81 (d, J=11.9 Hz, 3H), 1.62-1.52 (m, 6H).

### Example 341

¹H NMR: (400 MHz, CD₃OD) δ 8.13-8.04 (m, 2H), 7.68-7.53 (m, 2H), 7.47-7.38 (m, 1H), 5.37 (d, J=13.2 Hz, 1H), 4.61-4.49 (m, 2H), 4.36-4.23 (m, 1H), 4.11-4.05 (m, 1H), 3.79-3.72 (m, 2H), 3.71-3.54 (m, 4H), 3.53-3.47 (m, 2H), 3.44-3.39 (m, 1H), 3.22-3.13 (m, 1H), 2.94-2.82 (m, 1H), 2.12-1.97 (m, 2H), 1.92-1.75 (m, 6H), 1.62-1.52 (m, 3H), 1.25-1.16 (m, 3H).

### Example 342

¹H NMR: (400 MHz, CD₃OD) δ 8.20-8.05 (m, 2H), 7.71-7.53 (m, 2H), 7.53-7.39 (m, 1H), 5.45-5.29 (m, 1H), 4.70-4.40 (m, 2H), 4.30-4.09 (m, 2H), 4.07-3.77 (m, 1H), 3.68-3.61 (m, 2H), 3.59-3.51 (m, 2H), 3.48-3.40 (m, 2H), 3.36-3.34 (m, 1H), 3.29-3.22 (m, 1H), 3.18-3.03 (m, 1H), 2.98-2.82 (m, 1H), 2.31-2.17 (m, 1H), 1.92-1.63 (m, 7H), 1.60-1.51 (m, 3H), 1.02-0.83 (m, 3H).

### Example 350a1

¹H NMR (400 MHz, CD₃OD) δ 7.67-7.62 (m, 1H), 7.29-7.19 (m, 2H), 7.04 (dd, J=33.7, 2.5 Hz, 1H), 5.21 (d, J=12.7 Hz, 1H), 4.56 (dd, J=8.6, 5.5 Hz, 4H), 3.87 (d, J=11.4 Hz, 1H), 3.41 (dd, J=16.2, 4.4 Hz, 1H), 3.25-3.16 (m, 2H), 3.15-3.10 (m, 2H), 3.03-2.87 (m, 3H), 2.62-2.51 (m, 2H), 2.47-2.41 (m, 1H), 2.32-2.16 (m, 2H), 2.04 (d, J=8.9 Hz, 1H), 1.87 (d, J=17.4 Hz, 2H), 1.77 (d, J=13.4 Hz, 1H), 1.60 (s, 1H), 1.32 (s, 3H), 0.87 (dd, J=16.1, 8.5 Hz, 3H).

### Example 350a2

¹H NMR (400 MHz, CD₃OD) δ 7.68-7.60 (m, 1H), 7.27-7.20 (m, 2H), 7.04 (dd, J=30.2, 2.6 Hz, 1H), 5.17 (d, J=13.0 Hz, 1H), 4.56 (dd, J=8.7, 5.6 Hz, 4H), 3.89 (t, J=12.2 Hz, 1H), 3.42 (dd, J=19.2, 6.7 Hz, 1H), 3.19-3.10 (m, 4H), 2.98 (dd, J=16.3, 4.7 Hz, 2H), 2.88 (d, J=11.1 Hz, 1H), 2.62-2.52 (m, 2H), 2.46 (dd, J=13.8, 6.5 Hz, 1H), 2.31-2.15 (m, 2H), 2.04 (dd, J=12.1, 6.5 Hz, 1H), 1.87 (d, J=17.6 Hz, 2H), 1.76 (d, J=12.4 Hz, 1H), 1.60 (s, 1H), 1.30 (s, 3H), 0.87 (dd, J=13.4, 7.2 Hz, 3H).

### Example 350b1

¹H NMR (400 MHz, CD₃OD) δ 7.65 (dd, J=9.1, 5.7 Hz, 1H), 7.30-7.21 (m, 2H), 7.06 (dd, J=26.7, 2.7 Hz, 1H), 5.17 (s, 1H), 4.56 (d, J=3.3 Hz, 4H), 3.95 (s, 1H), 3.42 (d, J=10.0 Hz, 4H), 3.20-3.12 (m, 3H), 2.89 (s, 1H), 2.68-2.50 (m, 3H), 2.30 (s, 1H), 2.21-2.09 (m, 2H), 1.9-1.93 (m, 4H), 1.23-1.16 (m, 3H), 0.91-0.83 (m, 3H).

### Example 350b2

¹H NMR (400 MHz, CD₃OD) δ 7.65 (dd, J=9.1, 5.8 Hz, 1H), 7.28-7.20 (m, 2H), 7.06 (dd, J=28.2, 2.7 Hz, 1H), 5.02 (s, 1H), 4.58 (s, 4H), 3.94 (s, 1H), 3.44 (d, J=31.8 Hz, 2H), 3.17 (s, 3H), 3.03-2.95 (m, 2H), 2.89-2.82 (m, 1H), 2.57 (dd, J=14.8, 6.9 Hz, 2H), 2.49-2.35 (m, 2H), 2.06 (s, 2H), 1.98-1.90 (m, 4H), 1.17 (d, J=15.7 Hz, 3H), 0.91-0.83 (m, 3H).

### Example 355

¹H NMR (400 MHz, CD₃OD) δ 7.64 (dd, J=9.0, 5.9 Hz, 1H), 7.27-7.20 (m, 2H), 7.04 (dd, J=16.9, 2.5 Hz, 1H), 5.04 (s, 1H), 4.93 (d, J=4.9 Hz, 1H), 4.55-4.50 (m, 1H), 3.92 (s, 1H), 3.41 (dd, J=16.4, 4.2 Hz, 1H), 3.16 (d, J=8.5 Hz, 4H), 3.01-2.92 (m, 2H), 2.85 (d, J=7.3 Hz, 1H), 2.60-2.55 (m, 1H), 2.52-2.38 (m, 2H), 2.29-2.11 (m, 1H), 2.14-1.90 (m, 4H), 1.86 (d, J=12.2 Hz, 3H), 1.58 (d, J=7.9 Hz, 2H), 1.46 (dd, J=6.4, 5.0 Hz, 3H), 0.94-0.81 (m, 3H).

### Example 356

¹H NMR (400 MHz, CD₃OD) δ 8.15-8.08 (m, 2H), 7.71-7.52 (m, 2H), 7.50-7.42 (m, 1H), 5.47 (d, J=12.9 Hz, 1H), 4.66-4.36 (m, 2H), 4.13-3.75 (m, 2H), 3.63-3.33 (m, 3H), 3.13-3.01 (m, 1H), 2.40 (t, J=10.6 Hz, 6H), 2.14 (dd, J=15.7, 8.3 Hz, 1H), 1.82-1.69 (m, 3H), 1.56 (dd, J=10.3, 4.4 Hz, 3H), 1.33 (s, 2H), 1.11-0.99 (m, 2H).

### Example 357

¹H NMR (400 MHz, CD₃OD) δ 8.14-7.99 (m, 2H), 7.66-7.48 (m, 2H), 7.42 (q, J=8.9 Hz, 1H), 5.41-5.15 (m, 1H), 4.63-4.45 (m, 1H), 4.06 (d, J=8.6 Hz, 1H), 3.85-3.47 (m, 3H), 3.28-3.09 (m, 2H), 3.02-2.89 (m, 1H), 2.81-2.66 (m, 2H), 2.62-2.50 (m, 1H), 2.39 (s, 3H), 2.23-2.13 (m, 1H), 2.05-1.94 (m, 1H), 1.91-1.74 (m, 6H), 1.57 (t, J=7.0 Hz, 3H).

### Example 358

¹H NMR (400 MHz, CD₃OD) δ 8.16-8.00 (m, 2H), 7.73-7.51 (m, 2H), 7.42 (q, J=8.9 Hz, 1H), 5.41-5.20 (m, 1H), 4.62-4.48 (m, 1H), 4.05 (d, J=8.4 Hz, 1H), 3.83-3.41 (m, 3H), 3.27-3.08 (m, 1H), 3.01-2.76 (m, 2H), 2.35 (s, 6H), 2.09-1.97 (m, 1H), 1.92-1.73 (m, 3H), 1.68 (d, J=2.0 Hz, 6H), 1.56 (t, J=6.8 Hz, 3H).

### Example 359

¹H NMR (400 MHz, CD₃OD) δ 8.14-8.06 (m, 2H), 7.71-7.54 (m, 2H), 7.51-7.39 (m, 2H), 5.38 (m, 1H), 4.77-4.53 (m, 1H), 4.52-4.23 (m, 2H), 4.16-3.77 (m, 2H), 3.58 (d, J=34.7 Hz, 2H), 3.19-3.07 (m, 1H), 3.07-3.00 (m, 1H), 2.81 (d, J=22.2 Hz, 1H), 2.34-2.08 (m, 5H), 1.90-1.66 (m, 7H), 1.57 (dd, J=10.9, 6.3 Hz, 3H).

### Example 360

¹H NMR (400 MHz, CD₃OD) δ 8.12 (q, J=5.9 Hz, 2H), 7.71-7.55 (m, 2H), 7.46 (q, J=8.9 Hz, 1H), 5.37 (d, J=13.2 Hz, 1H), 4.46 (d, J=9.4 Hz, 1H), 4.28 (d, J=49.1 Hz, 2H), 4.11 (d, J=9.9 Hz, 1H), 3.62 (s, 1H), 3.53 (s, 1H), 3.48 (s, 1H), 3.11 (d, J=13.4 Hz, 1H), 3.06 (s, 2H), 2.69 (s, 2H), 2.22 (s, 1H), 1.95 (d, J=17.7 Hz, 3H), 1.90-1.82 (m, 3H), 1.73 (dd, J=30.1, 12.3 Hz, 7H), 1.57 (dd, J=6.3, 3.7 Hz, 3H).

### Example 361

¹H NMR (400 MHz, CD₃OD) δ 7.55 (m, 1H), 7.20-7.10 (m, 2H), 6.94 (dd, J=50.8, 2.7 Hz, 1H), 5.13 (dd, J=13.1, 7.0 Hz, 1H), 4.86 (t, J=5.2 Hz, 1H), 4.48 (dd, J=8.7, 6.3 Hz, 1H), 3.93 (dd, J=9.1, 5.0 Hz, 1H), 3.54-3.48 (m, 1H), 3.32 (dd, J=16.2, 4.3 Hz, 1H), 3.17-2.99 (m, 4H), 2.89 (dd, J=15.9, 9.3 Hz, 3H), 2.77 (d, J=12.4 Hz, 1H), 2.46-2.41 (m, 2H), 2.39-2.32 (m, 1H), 2.13-2.04 (m, 1H), 2.03-1.93 (m, 2H), 1.81 (dt, J=13.6, 6.9 Hz, 1H), 1.76-1.65 (m, 1H), 1.46 (dd, J=6.4, 2.3 Hz, 5H), 1.28 (d, J=1.8 Hz, 3H), 0.87 (t, J=7.4 Hz, 1H), 0.71 (t, J=7.4 Hz, 2H).

### Example 362

¹H NMR (400 MHz, CD₃OD) δ 7.68-7.61 (m, 1H), 7.28-7.19 (m, 2H), 7.03 (dd, J=51.5, 2.5 Hz, 1H), 5.23 (dd, J=13.1, 6.9 Hz, 1H), 4.93 (s, 1H), 4.62-4.55 (m, 2H), 4.06-4.00 (m, 1H), 3.65 (dd, J=27.4, 10.9 Hz, 3H), 3.52 (d, J=15.9 Hz, 1H), 3.45-3.33 (m, 2H), 3.09-2.95 (m, 4H), 2.84 (d, J=13.4 Hz, 1H), 2.53-2.42 (m, 2H), 2.24-2.10 (m, 3H), 1.87 (m, 2H), 1.57 (t, J=8.3 Hz, 5H), 1.39 (d, J=1.7 Hz, 3H), 0.95 (d, J=7.4 Hz, 1H), 0.90-0.79 (m, 2H).

### Example 363

¹H NMR: (400 MHz, CD₃OD) δ 7.81-7.74 (m, 1H), 7.42-7.31 (m, 2H), 7.26-7.10 (m, 1H), 5.31-5.24 (m, 1H), 4.95-4.90 (m, 1H), 4.85-4.82 (m, 2H), 4.62-4.53 (m, 1H), 4.11-4.03 (m, 1H), 3.76-3.57 (m, 4H), 3.54-3.36 (m, 3H), 3.26-3.17 (m, 1H), 3.09-2.96 (m, 3H), 2.86-2.75 (m, 1H), 2.52-2.38 (m, 1H), 2.20-2.00 (m, 2H), 1.93-1.77 (m, 3H), 1.63-1.51 (m, 4H).

### Example 364

¹H NMR: (400 MHz, CD₃OD) δ 7.70-7.62 (m, 1H), 7.32-7.21 (m, 2H), 7.11-6.95 (m, 1H), 5.43-5.36 (m, 1H), 4.71-4.59 (m, 1H), 4.54-4.35 (m, 2H), 4.14-4.08 (m, 1H), 3.66-3.60 (m, 1H), 3.56-3.51 (m, 1H), 3.14-3.01 (m, 2H), 2.84-2.72 (m, 1H), 2.71-2.45 (m, 1H), 2.41-2.18 (m, 6H), 1.94-1.86 (m, 1H), 1.83-1.67 (m, 6H), 1.59-1.52 (m, 3H), 1.03-0.85 (m, 3H).

### Example 366

¹H NMR (400 MHz, CD₃OD) δ 8.13-8.04 (m, 2H), 7.67-7.53 (m, 2H), 7.42 (q, J=9.0 Hz, 1H), 5.55 (td, J=6.7, 6.2, 3.0 Hz, 1H), 5.42-5.31 (m, 1H), 4.70-4.48 (m, 3H), 4.08 (dd, J=8.9, 1.4 Hz, 1H), 3.71-3.66 (m, 1H), 3.60 (d, J=5.3 Hz, 1H), 3.23-3.12 (m, 1H), 3.04 (m, 1H), 2.92-2.81 (m, 2H), 2.59 (d, J=8.2 Hz, 1H), 2.49-2.42 (m, 1H), 2.41 (d, J=2.4 Hz, 3H), 2.13-1.99 (m, 2H), 1.86-1.76 (m, 2H), 1.57 (dd, J=8.2, 6.4 Hz, 3H).

### Example 367

¹H NMR (400 MHz, CD₃OD) δ 8.10 (dd, J=8.2, 5.6 Hz, 2H), 7.70-7.50 (m, 2H), 7.45 (q, J=8.7 Hz, 1H), 5.55-5.33 (m, 1H), 4.76 (s, 2H), 4.59 (s, 1H), 4.46 (m, 1H), 4.32 (d, J=12.9 Hz, 1H), 4.13-4.08 (m, 1H), 3.58 (d, J=41.9 Hz, 2H), 3.11 (dd, J=27.2, 13.0 Hz, 1H), 2.93 (s, 1H), 2.66-2.51 (m, 1H), 2.31 (d, J=18.4 Hz, 3H), 2.17 (t, J=9.5 Hz, 1H), 1.96-1.85 (m, 1H), 1.79 (s, 2H), 1.68 (d, J=8.5 Hz, 2H), 1.57 (d, J=5.8 Hz, 4H), 0.98 (d, J=11.9 Hz, 3H).

### Example 368

¹H NMR: (400 MHz, CD₃OD) δ 7.71-7.60 (m, 1H), 7.33-7.19 (m, 2H), 7.13-6.94 (m, 1H), 5.43-5.34 (m, 1H), 5.18-5.01 (m, 1H), 4.66-4.57 (m, 1H), 4.56-4.41 (m, 2H), 4.15-4.08 (m, 1H), 3.75-3.63 (m, 2H), 3.56-3.41 (m, 2H), 3.14-3.00 (m, 2H), 2.69-2.49 (m, 2H), 2.41-2.34 (m, 3H), 2.28-2.08 (m, 2H), 2.00-1.86 (m, 1H), 1.84-1.71 (m, 3H), 1.60-1.50 (m, 3H), 1.05-0.85 (m, 3H).

### Example 369

¹H NMR (400 MHz, CD₃OD) δ 7.64 (m, 1H), 7.29-7.19 (m, 2H), 7.03 (dd, J=51.2, 2.7 Hz, 1H), 5.65-5.54 (m, 1H), 5.38-5.32 (m, 1H), 4.56 (m, 2H), 4.13-4.04 (m, 1H), 3.69 (d, J=5.4 Hz, 1H), 3.59 (s, 1H), 3.19 (d, J=12.6 Hz, 1H), 3.11-3.03 (m, 1H), 2.97-2.87 (m, 2H), 2.67-2.59 (m, 1H), 2.52-2.46 (m, 1H), 2.45 (s, 3H), 2.20-2.00 (m, 3H), 1.91-1.75 (m, 3H), 1.56 (dd, J=6.4, 2.2 Hz, 3H), 1.31 (d, J=17.5 Hz, 3H).

### Example 370

¹H NMR: (400 MHz, CD₃OD) δ 8.12-8.04 (m, 2H), 7.68-7.52 (m, 2H), 7.47-7.38 (m, 1H), 5.37-5.29 (m, 1H), 4.97-4.90 (m, 1H), 4.59-4.48 (m, 1H), 4.11-4.05 (m, 1H), 3.84-3.50 (m, 3H), 3.24-3.13 (m, 1H), 2.93-2.85 (m, 1H), 2.43-2.36 (m, 3H), 2.35-2.19 (m, 3H), 2.09-2.00 (m, 1H), 1.94-1.72 (m, 5H), 1.61-1.49 (m, 4H), 1.28-1.21 (m, 3H).

### Example 371a

¹H NMR (400 MHz, CD₃OD) δ 8.15-8.01 (m, 2H), 7.68-7.51 (m, 2H), 7.42 (dd, J=19.0, 9.0 Hz, 1H), 5.80-5.62 (m, 1H), 5.45-5.32 (m, 1H), 4.61-4.45 (m, 1H), 4.13-4.02 (m, 1H), 3.82-3.47 (m, 3H), 3.24-3.14 (m, 1H), 2.82-2.71 (m, 1H), 2.64-2.55 (m, 1H), 2.55-2.47 (m, 1H), 2.33-2.26 (m, 1H), 2.22 (d, J=0.9 Hz, 3H), 2.06-1.99 (m, 1H), 1.91-1.73 (m, 3H), 1.69-1.62 (m, 1H), 1.61-1.51 (m, 3H), 1.46-1.38 (m, 1H), 0.86-0.76 (m, 1H), 0.74-0.60 (m, 1H).

### Example 371b

¹H NMR (400 MHz, CD₃OD) δ 8.17-7.99 (m, 2H), 7.68-7.52 (m, 2H), 7.42 (dd, J=17.2, 8.8 Hz, 1H), 5.77-5.63 (m, 1H), 5.38 (m, 1H), 4.58-4.51 (m, 1H), 4.09 (d, J=8.6 Hz, 1H), 3.82-3.49 (m, 3H), 3.22-3.13 (m, 1H), 2.86-2.78 (m, 1H), 2.69-2.59 (m, 1H), 2.54-2.44 (m, 1H), 2.32-2.20 (m, 4H), 2.11-2.03 (m, 1H), 1.93-1.76 (m, 3H), 1.68-1.62 (m, 1H), 1.57 (dd, J=8.1, 6.5 Hz, 3H), 1.46-1.38 (m, 1H), 0.84-0.77 (m, 1H), 0.73-0.63 (m, 1H).

### Example 372a

¹H NMR (400 MHz, CD₃OD) δ 8.14-8.00 (m, 2H), 7.67-7.53 (m, 2H), 7.42 (q, J=9.0 Hz, 1H), 5.34 (m, 1H), 5.07 (m, 1H), 4.54 (dd, J=8.6, 6.5 Hz, 1H), 4.08 (d, J=8.7 Hz, 1H), 3.79-3.50 (m, 3H), 3.22-3.15 (m, 1H), 3.08 (t, J=8.5 Hz, 1H), 2.61-2.53 (m, 2H), 2.45-2.34 (m, 4H), 2.03 (d, J=3.4 Hz, 1H), 1.94-1.85 (m, 2H), 1.85-1.75 (m, 2H), 1.57 (dd, J=8.3, 6.5 Hz, 3H), 1.30 (dd, J=6.4, 2.4 Hz, 3H).

### Example 372b

¹H NMR (400 MHz, CD₃OD) δ 8.11-8.03 (m, 2H), 7.66-7.52 (m, 2H), 7.42 (q, J=9.1 Hz, 1H), 5.58 (m, 1H), 5.35 (m, 1H), 4.52 (m, 1H), 4.06 (dd, J=8.7, 1.4 Hz, 1H), 3.80-3.47 (m, 3H), 3.20-3.11 (m, 2H), 2.53-2.43 (m, 2H), 2.32 (d, J=1.6 Hz, 3H), 2.23 (m, 1H), 2.05-1.73 (m, 5H), 1.56 (dd, J=8.6, 6.4 Hz, 3H), 1.23 (dd, J=6.5, 1.6 Hz, 3H).

### Example 373a

¹H NMR (400 MHz, CD₃OD) δ 7.69-7.60 (m, 1H), 7.29-7.17 (m, 2H), 7.13-6.93 (m, 1H), 5.45-5.36 (m, 1H), 4.63-4.51 (m, 2H), 4.36 (q, J=10.5 Hz, 2H), 4.15-4.05 (m, 1H), 3.69 (d, J=5.6 Hz, 1H), 3.59 (s, 1H), 3.24-3.10 (m, 1H), 2.64-2.50 (m, 3H), 2.37 (s, 6H), 2.20-2.01 (m, 2H), 1.91-1.71 (m, 3H), 1.60-1.53 (m, 3H), 1.32 (s, 3H), 0.96 (t, J=7.3 Hz, 1H), 0.79 (t, J=7.4 Hz, 2H).

### Example 373b

¹H NMR (400 MHz, CD₃OD) δ 7.69-7.60 (m, 1H), 7.31-7.21 (m, 2H), 7.03 (dd, J=52.6, 2.6 Hz, 1H), 5.46-5.27 (m, 1H), 4.62-4.49 (m, 1H), 4.42-4.27 (m, 2H), 4.16-4.00 (m, 1H), 3.73-3.66 (m, 1H), 3.59 (s, 1H), 3.25-3.10 (m, 2H), 2.62-2.45 (m, 3H), 2.37 (s, 6H), 2.22-2.03 (m, 2H), 1.91-1.75 (m, 3H), 1.56 (d, J=6.3 Hz, 3H), 1.32 (s, 3H), 0.96 (t, J=7.3 Hz, 1H), 0.79 (t, J=7.4 Hz, 2H).

### Example 374

¹H NMR (400 MHz, CD₃OD) δ 8.51 (s, 1H), 7.70-7.60 (m, 1H), 7.31-7.20 (m, 2H), 7.13-6.93 (m, 1H), 5.31 (d, J=13.5 Hz, 1H), 4.64-4.48 (m, 1H), 4.11 (d, J=7.9 Hz, 1H), 3.89 (dd, J=21.6, 11.6 Hz, 1H), 3.72 (d, J=31.9 Hz, 2H), 3.51-3.39 (m, 2H), 3.26-3.17 (m, 1H), 2.83-2.72 (m, 3H), 2.55-2.29 (m, 3H), 2.22-2.14 (m, 1H), 2.07-1.97 (m, 2H), 1.95-1.90 (m, 1H), 1.87 (s, 3H), 1.82 (d, J=5.9 Hz, 1H), 1.57 (d, J=6.2 Hz, 3H), 0.97 (t, J=7.3 Hz, 1H), 0.81 (t, J=7.3 Hz, 2H).

### Example 375

¹H NMR (400 MHz, CD₃OD) δ 8.16-8.00 (m, 2H), 7.73-7.51 (m, 2H), 7.42 (q, J=8.9 Hz, 1H), 5.41-5.20 (m, 1H), 4.62-4.48 (m, 1H), 4.05 (d, J=8.4 Hz, 1H), 3.83-3.41 (m, 3H), 3.27-3.08 (m, 1H), 3.01-2.76 (m, 2H), 2.35 (s, 6H), 2.09-1.97 (m, 1H), 1.92-1.73 (m, 3H), 1.68 (d, J=2.0 Hz, 6H), 1.56 (t, J=6.8 Hz, 3H).

### Example 376a

¹H NMR (400 MHz, CD₃OD) δ 7.68-7.61 (m, 1H), 7.24 (m, 2H), 7.03 (dd, J=52.1, 2.5 Hz, 1H), 5.36-5.31 (m, 1H), 5.11-5.07 (m, 1H), 4.56 (d, J=1.9 Hz, 1H), 4.12-4.05 (m, 1H), 3.70 (d, J=5.3 Hz, 1H), 3.60 (s, 1H), 3.19 (t, J=12.0 Hz, 1H), 3.09 (t, J=8.6 Hz, 1H), 2.62-2.56 (m, 2H), 2.53-2.45 (m, 1H), 2.41 (s, 3H), 2.22-2.15 (m, 1H), 2.02 (d, J=6.5 Hz, 1H), 1.92-1.88 (m, 1H), 1.80 (d, J=8.0 Hz, 1H), 1.58-1.52 (m, 3H), 1.31 (dd, J=6.4, 2.3 Hz, 6H), 0.96 (t, J=7.3 Hz, 1H), 0.90-0.78 (m, 2H).

### Example 376b

¹HNMR (400 MHz, CD₃OD) δ 7.67-7.62 (m, 1H), 7.24 (m, 2H), 7.03 (dd, J=53.9, 2.5 Hz, 1H), 5.61 (d, J=7.8 Hz, 1H), 5.36-5.31 (m, 1H), 4.59-4.51 (m, 1H), 4.10-4.04 (m, 1H), 3.67 (d, J=5.0 Hz, 1H), 3.58 (s, 1H), 3.25-3.13 (m, 2H), 2.54 (m, 3H), 2.39 (s, 3H), 2.36-2.30 (m, 1H), 2.23-2.10 (m, 1H), 2.04-1.96 (m, 2H), 1.87-1.74 (m, 3H), 1.55 (d, J=6.3 Hz, 3H), 1.34-1.23 (m, 6H).

### Biology Assay

The following assays were used to measure the effects of the compounds of the present disclosure.

### Phospho-ERK 1/2 Assay:

Allow PNAC-1 cell growth in a T75 flask in DMEM and 10% 1 fetal calf serum (FCS; Gibco®), using standard tissue culture procedures until ~80% confluency is achieved. Day 1, seed 6000 cells/well in 384 well plate and incubate at 37° C., 5% CO2. Add diluted compound by Echo 550, final DMSO is 0.5%, incubate cells at 37° C., 5% CO2 for 3 hours. Then, remove medium and fix cells with 3.7% formaldehyde in PBS (PFA) by Apricot. Wash with PBS once. Permeabilize cells with cold 100% methanol and repeat wash once with PBS once. Add Li-Cor blocking buffer to each well and incubate 1.5 hours at RT. Remove blocking buffer and add primary antibody mixture (rabbit anti pERK, mouse anti GAPDH). Incubate at 4° C. overnight. Day 2, wash with PBST (Tween-20 in PBS) with total 3 times and then add secondary antibody mixture (goat anti rabbit 800CW (1:800 dilution in the combined solution) and goat anti mouse 680RD (1:800 dilution in the combined solution)), incubate for 60 minutes at RT away from light. Repeat washing with PBST 3 times. After final wash, centrifuge plate up-side-down at 1000 rpm to remove wash solution completely from wells. Before plate scanning, clean the bottom plate surface and the Odyssey© Imager scanning bed (if applicable) with moist, lint-free tissue to avoid any obstructions during scanning. Scan plate with detection in both 700 and 800 nm channels.

### KRAS(G12D): SOS1 Nucleotide Exchange Binding Assay

Thaw GDP loaded KRAS(G12D) on ice and dilute GDP loaded KRAS(G12D) to 500 nM in RBD-RAS binding buffer. Prepare the master mixture (6 μl): 96 wells×(1 μl diluted GDP loaded KRAS(G12D), 500 nM+5 μl RBD-RAS binding buffer). Add 6 μl of master mix to each well. Prepare serial dilutions of the test compound in DMSO at 200× testing concentration. Then dilute the compound 20-fold in deionized water to prepare the 10× intermediate solution. For positive and negative controls, use 5% DMSO in water as a 10× intermediate so that all wells contain the same amount of DMSO. Add 1 μl of 10× intermediate solution of the test compound to the testing wells. Add 1 μl 5% DMSO to the positive and negative control wells. Briefly centrifuge the plate and incubate for 30 minutes at room temperature. Thaw GTP (10 mM) and SOS1 on ice. Dilute SOS1 in RBD-RAS binding buffer at 5 μM. Combine GTP (10 mM) and diluted SOS1 (5 μM) at 1:1 ratio. Initiate the exchange reaction by adding 2 μl of GTP/SOS1 mixture to the testing and the positive control wells. Thaw RBD-cRAF and dilute RBD-cRAF in RBD-RAS binding buffer at 25 nM. After the 30-minute incubation with SOS1/GTP (RBD-RAS buffer for the negative control), add 1 μl of diluted RBD-cRAF (25 nM) to all wells. Briefly centrifuge the plate and incubate at room temperature for 30 minutes. Dilute 3× Immuno Buffer in deionized water to prepare 1× Immuno buffer. Add one volume of 3× Immuno Buffer to two volumes of deionized water. Dilute Glutathione Acceptor beads (PerkinElmer #AL109C) and Nickel chelate Donor beads (PerkinElmer #AS101D) at 1:500 and 1:250 respectively in 1× Immuno buffer. Add 20 μl of acceptor/donor beads mixture per well is needed. Therefore add 16 μl of Glutathione Acceptor beads and 32 μl of Nickel Donor beads to 8 mL of 1× Immuno buffer). Incubate 30 min at room temperature. Read Alpha-counts using a compatible plate reader.

### KRAS G12D 2D CellTiter-Glo® Proliferation Assay:

AsPC-1 (ATCC CRL-1682) and LS513 (ATCC CRL-2134) cells were purchased from ATCC, GP2D (Cobioer CBP60010), AGS (Cobioer CBP60476), SW1990 (Cobioer CBP60691) cells were purchased from Cobioer biosciences CO., LTD, MKN-1 (JCRB, JCR130252) cells was purchased from JCRB cell bank. Each cell was cultured in medium supplemented with 10% fetal bovine serum (FBS), according to the protocol recommended by the manufacture. Cells were seeded 800 cells/well in 384-well plates (Corning) and incubated at 37° C., 5% CO₂ for 18 hours. Serially diluted compound was added to the cells, and plates were incubated at 37° C., 5% CO₂ for 72 hours. Cell viability was measured using a CellTiter-Glo® Luminescent Cell Viability Assay kit (Promega) according to the manufacturer's protocol.

### KRAS G12D 3D CellTiter-Glo® Proliferation Assay:

HPAC (ATCC CRL-2119), Panc0403 (ATCC CRL-2555) and AsPC-1 (ATCC CRL-1682) cells were purchased from ATCC, GP2D (Cobioer CBP60010), AGS (Cobioer CBP60476), SW1990 (Cobioer CBP60691) cells were purchased from Cobioer biosciences CO., LTD, MKN-1 (JCRB, JCRB0252) cells was purchased from JCRB cell bank. Each cell was cultured in medium supplemented with 10% fetal bovine serum (FBS), according to the protocol recommended by the manufacture. Serially diluted compound was added to 384-well Ultra-Low Attachment Surface round bottom plate (Corning). 400 cells/well were seeded in plate and incubated at 37° C., 5% CO₂ for 7 days. Cell viability was measured using a CellTiter-Glo® 3D Cell Viability Assay kit (Promega) according to the manufacturer's protocol.

The Phospho-ERK 1/2 assay results for some exemplary compounds of the present disclosure are shown in Table 1 below.

TABLE 1

| Example No. | PANC-1 pERK IC50 nM |
|---|---|
| 1 | 386 |
| 2 | 15 |
| 23 | 96 |
| 24 | 85 |
| 45 | 23 |
| 46a | 5.9 |
| 46b | 28 |
| 47 | 629 |
| 199 | 37 |
| 200a | 12 |
| 200b | 697 |
| 202b | 990.7 |
| 222 | 113 |
| 227 | 5.2 |
| 228 | 19 |
| 330 | 171 |
| 332 | 778 |
| 341 | 70 |
| 350a1 | 151 |
| 357 | 123 |
| 361 | 43 |
| 362 | 221 |
| 363 | 88 |
| 365a | 6.0 |
| 365b | 110 |
| 366 | 272 |
| 367 | 234 |
| 369 | 400 |
| 370 | 271 |
| 371a | 304 |
| 371b | 794 |
| 372a | 391 |
| 372b | 70 |
| 373a | 6.4 |
| 373b | 137 |
| 376a | 145 |
| 376b | 133 |

Other compounds of the present disclosure show IC₅₀ value of 0.5 to 5000 nM. Some compounds of the present disclosure show IC₅₀ value of 1-4000 nM. Some compounds of the present disclosure show IC₅₀ value of 1-3000 nM. Some compounds of the present disclosure show IC₅₀ value of 1-2000 nM. Some compounds of the present disclosure show IC₅₀ value of 1-1000 nM. Some compounds of the present disclosure show IC₅₀ value of 1-500 nM.

### DMPK Assay

### Caco-2 Cell Monolayers Permeability:

10 μM input concentration and pH 6.5/7.4 (apical/basolateral), in the presence of efflux inhibitors, Zosuquidar, Benzbromarone and KO-143. Incubations were performed at 37° C. with shaking at 480 rpm on a rotary shaker over the course of 120 minutes, and samples were collected at 45 and 120 minutes to assess recovery. All incubations were performed in singlet. Lucifer yellow was used as a marker to confirm the integrity of the cell monolayers following 120 min of incubation. UPLC-MS/MS was used to quantify compound concentrations in the incubation medium of donor and receiver compartments. The concentration data was used to calculate the apparent permeability after 120 min of incubation. Results are shown in Table 2.

TABLE 2

| Example No. | Papp (×10⁻⁶ cm/s) |
|---|---|
| MRTX1133 | <0.15 |
| 2 | 1.0 |
| 199 | 3.17 |
| 200a | 1.49 |
| 227 | 1.06 |

### MDCK-MDR1 Pgp Assessment

Efflux transport mediated by P-glycoprotein (Pgp) was assessed by MDCK-MDR1 cells. The final concentrations of test compounds and control compound were at 1 μM. The multi-well insert plate was incubated at 37 ⁹C for 2 hours. In order to reliably measure passive permeability coefficients, Pexact was reported, which automatically corrects for loss of compound. Results are shown in Table 3.

TABLE 3

| Example No. | Pgp Efflux Ratio (Pexact) |
|---|---|
| 365a | 8.4 |
| 373a | 12.5 |

### In Vivo Oral Bioavailability Assay (Mouse PK):

Single dose following IV bolus (1 mg/kg, 0.2 mg/mL in 1% DMSO, 99% SBE-β-CD (10% w/v) in water) and oral gavage (10 mg/kg~60 mg/kg) administration of test compound in Balb/c Mice female. The blood samples were collected at 2 min, 5 min, 10 min, 30 min, 1 hr, 2 hr, 4 hr, 8 hr and 24 hr (additional 32 hr and 48 hr for MRTX1133) after IV bolus, at 15 min, 30 min, 1 hr, 1.5 hr, 2 hr, 3 hr, 4 hr, 8 hr and 24 hr (additional 32 hr and 48 hr for MRTX1133) after PO administration. The plasma concentrations of compounds were determined with UPLC-MS/MS. Results are shown in Table 4.

TABLE 4

| Example No. | Oral Dose | Fa*Fg (%) | Oral Bioavailability (%) |
|---|---|---|---|
| MRTX-1133 | 30 mpk | 0.95 | 0.66 |
| 200a | 10 mpk | 24 | 15.8 |

### In Vivo Oral Bioavailability Assay (Rat PK):

Single dose following IV infusion (1 mg/kg, 0.25 mg/mL) and oral gavage (5 mg/kg~60 mg/kg) administration of test compounds in SD Rats male. The blood samples were collected at 10 min, 30 min, 1 hr, 1.25 hr, 1.5 hr, 2 hr, 4 hr, 8 hr and 24 hr after IV infusion, at 15 min, 30 min, 1 hr, 1.5 hr, 2 hr, 3 hr, 4 hr, 8 hr and 24 hr after PO administration. The plasma concentrations of compounds were determined with UPLC-MS/MS.

The foregoing description is considered as illustrative only of the principles of the present disclosure. Further, since numerous modifications and changes will be readily apparent to those skilled in the art, it is not desired to limit the invention to the exact construction and process shown as described above. Accordingly, all suitable modifications and equivalents may be considered to fall within the scope of the invention as defined by the claims that follow.

What claimed is:

1. A compound having Formula (I) or Formula (I′): 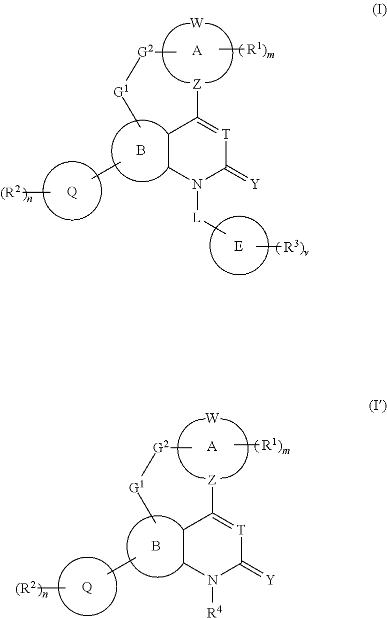
or a pharmaceutically acceptable salt thereof,
wherein
Y is O or S;
T is N or C(R^{T});
R^{T} is selected from hydrogen, halogen, hydroxyl, cyano, alkyl, heteroalkyl, haloalkyl or —OR^{b};
Ring A is heterocyclyl or heteroaryl,
Z is C(R^{e}) or N;
R^{e} is absent or hydrogen;
W is N(R^{1W}) or C(R^{2W})(R^{3W});
R^{1W} is absent, hydrogen, hydroxyl, alkyl, cycloalkyl, heterocyclyl or heteroalkyl;
R^{2W} is absent, hydrogen, alkyl or cycloalkyl;
R^{3W} is hydrogen, hydroxyl, —N(R^{a})₂, alkyl or heteroalkyl;
each R¹ is independently selected from the group consisting of hydrogen, oxo, hydroxyl, halogen, cyano, alkyl, alkenyl, alkynyl, alkylalkoxy, heteroalkyl, heteroaryl, —C(O)R*, —C(O)OR*, —C(O)N(R^{a})₂ and —N(R^{a})₂, wherein the alkyl, alkenyl, alkynyl, alkylalkoxy, heteroalkyl and heteroaryl are optionally substituted with one or more groups independently selected from deuterium, cyano, hydroxyl, halogen, —OR^{b}, or —N(R^{e})₂, provided that if R¹ is present, R¹ is not connected to W;
each R^{a} and R^{b} is independently hydrogen, alkyl, alkenyl or alkynyl, wherein the alkyl and alkenyl are optionally substituted with one or more deuterium;
R* is selected from hydrogen, alkyl, alkylaryl or aryl;
Ring B is selected from cycloalkyl, heterocyclyl, aryl, or heteroaryl, each of which is optionally substituted with one or more R′;
each R′ is independently selected from the group consisting of oxo, hydroxyl, halogen, cyano, amino, alkyl, alkenyl, alkynyl, heteroalkyl, heteroalkenyl, heteroalkynyl, heteroaryl, wherein alkyl, alkenyl, alkynyl, heteroalkyl, heteroalkenyl, heteroalkynyl and heteroaryl are optionally substituted with one or more group independently consisting of deuterium, hydroxyl, halogen, cyano, —OR^{a}, —N(R^{a})₂, and heteroaryl;
Ring Q is selected from cycloalkyl, heterocyclyl, aryl or heteroaryl;
each R² is independently selected from the group consisting of hydrogen, oxo, hydroxyl, halogen, cyano, amino, nitro, alkyl, alkenyl, alkynyl, alkoxy, haloalkyl, heteroalkyl, heteroalkenyl, heteroalkynyl, cycloalkyl, heterocyclyl, aryl, heteroaryl and —C(O)R*, wherein alkyl, alkenyl, alkynyl, alkoxy, heteroalkyl, heteroalkenyl, heteroalkynyl, cycloalkyl, heterocyclyl, aryl, and heteroaryl are optionally substituted with one or more group independently consisting of deuterium, hydroxyl, halogen, cyano, amino, nitro, alkyl, alkoxy, haloalkyl, hydroxyalkyl, cycloalkyl, heterocyclyl, aryl and heteroaryl;
G¹ is a bond, —[C(R^{d})₂]ᵤ—, —O—, —S(O)ₚ—, —S—S—, —N(R^{c})—, or —C(R^{d})═C(R^{d})—;
G² is a bond, —[C(R^{d})₂]ᵤ—, —N(R^{c})—, —C(O)— or —C(O)C(R^{d})₂—;
R^{c} is selected from the group consisting of hydrogen, alkyl, haloalkyl, hydroxyalkyl, heteroalkyl, cycloalkyl and heterocyclyl, wherein the alkyl, haloalkyl, hydroxyalkyl, heteroalkyl, cycloalkyl and heterocyclyl are optionally substituted with one or more deuterium;
each R^{d} is independently selected from the group consisting of hydrogen, hydroxyl, halogen, cyano, amino, nitro, alkyl, alkenyl, alkynyl, alkoxy, heteroalkyl, heteroalkenyl, heteroalkynyl, cycloalkyl, and heterocyclyl, wherein alkyl, alkenyl, alkynyl, alkoxy, heteroalkyl, heteroalkenyl, heteroalkynyl, cycloalkyl, and heterocyclyl are optionally substituted with one or more group independently consisting of deuterium, hydroxyl, halogen, cyano, amino, nitro, alkyl, alkoxy, haloalkyl, and hydroxyalkyl; or
two R^{d} together with the carbon atom to which they are both attached form a cycloalkyl or heterocyclyl, wherein cycloalkyl and heterocyclyl are optionally substituted with cyano, halogen, hydroxyl, amino, nitro, alkoxy, haloalkyl, hydroxyalkyl and alkyl;
L is selected from alkyl, alkenyl, alkynyl, alkylcycloalkylalkyl, or alkylheterocyclylalkyl, each of which is optionally substituted with one or more R^{f};
each R^{f} is independently selected from deuterium, alkyl, hydroxyl, halogen, cyano or amino, wherein the alkyl and amino are optionally substituted with one or more groups independently selected from deuterium, hydroxyl and alkyl;
Ring E is selected from cycloalkyl, heterocyclyl, aryl or heteroaryl;
each R³ is independently selected from the group consisting of hydrogen, deuterium, oxo, hydroxyl, halogen, alkyl, alkoxy, haloalkyl, hydroxyalkyl, cycloalkyl, heterocyclyl, —N(R^{g})₂ and ═C(R″)₂, wherein the alkyl, alkoxy, haloalkyl, hydroxyalkyl, cycloalkyl, heterocyclyl are optionally substituted with one or more groups selected from deuterium, cycloalkyl or heterocyclyl;
each R^{g} is independently hydrogen, alkyl, alkenyl or alkynyl;
each R″ is independently selected from hydrogen, hydroxyl, halogen, cyano, alkyl, cycloalkyl, hydroxyalkyl, haloalkyl or heterocyclyl;
R⁴ is alkyl optionally substituted with one or more group independently selected from deuterium, halogen, hydroxyl, hydroxyalkyl, alkyl, alkoxy, —N(R^{h})₂ or -alkyl-N(R^{h})₂;
each R^{h} is independently hydrogen, alkyl, alkenyl or alkynyl;
m is an integer from 0 to 6;
n is an integer from 0 to 5;
p is an integer from 0 to 2;
u is an integer from 0 to 4; and
v is an integer from 0 to 4.

2. The compound of claim 1, or a pharmaceutically acceptable salt thereof, wherein Z is C(R^{e}).

3. The compound of claim 2, or a pharmaceutically acceptable salt thereof, wherein R^{e} is absent.

4. The compound of claim 2, or a pharmaceutically acceptable salt thereof, wherein R^{e} is hydrogen.

5. The compound of claim 1, or a pharmaceutically acceptable salt thereof, wherein Z is N.

6. The compound of claim 1, or a pharmaceutically acceptable salt thereof, wherein W is N(R^{1W}).

7. The compound of claim 6, or a pharmaceutically acceptable salt thereof, wherein R^{1W} is hydrogen.

8. The compound of claim 1, or a pharmaceutically acceptable salt thereof, wherein W is C(R^{2W})(R^{3W}).

9. The compound of claim 8, or a pharmaceutically acceptable salt thereof, wherein R^{2W} is hydrogen, R^{3W} is hydrogen, —N(R^{a})₂, hydroxyl or alkyl.

10. The compound of any one of preceding claims, or a pharmaceutically acceptable salt thereof, wherein Ring A is heteroaryl.

11. The compound of any one of claims 1-9, or a pharmaceutically acceptable salt thereof, wherein Ring A is heterocyclyl.

12. The compound of claim 11, or a pharmaceutically acceptable salt thereof, wherein Ring A is selected from the group consisting of: 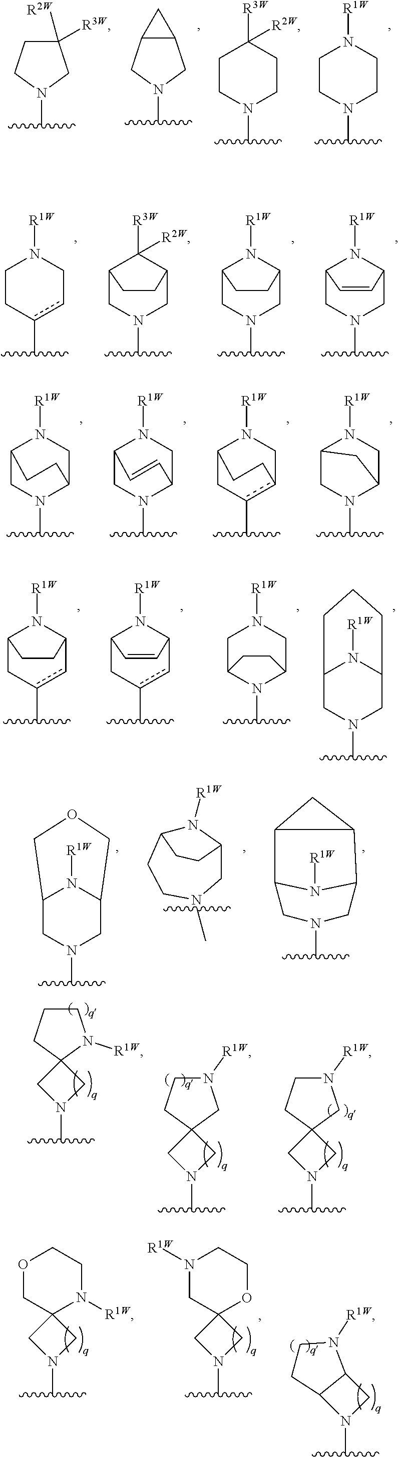 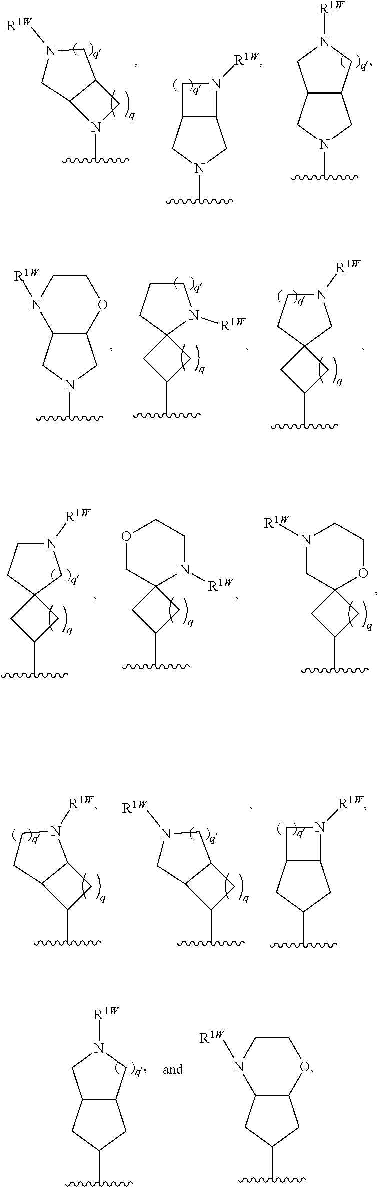
wherein [symbol] represents a single bond or a double bond, q is an integer from 1 to 4, and q′ is an integer from 0 to 4.

13. The compound of claim 12, or a pharmaceutically acceptable salt thereof, wherein Ring A is selected from the group consisting of: 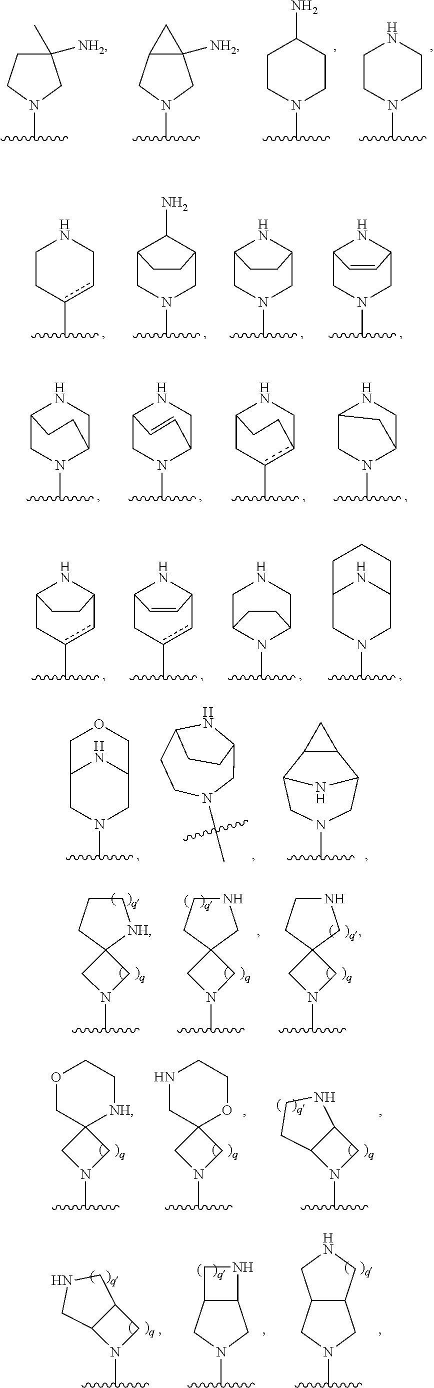 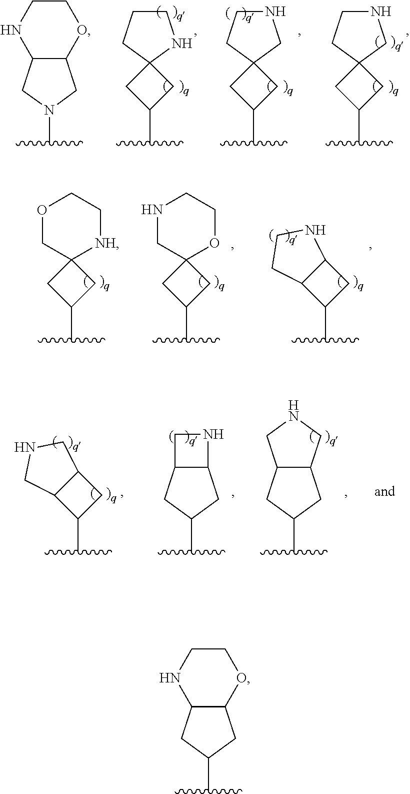
wherein [symbol] represents a single bond or a double bond, q is an integer from 1 to 4, and q′ is an integer from 0 to 4.

14. The compound of any one of preceding claims, or a pharmaceutically acceptable salt thereof, wherein Ring B is cycloalkyl optionally substituted with one or more R′.

15. The compound of any one of claims 1-13, or a pharmaceutically acceptable salt thereof, wherein Ring B is heterocyclyl optionally substituted with one or more R′.

16. The compound of claim 15, or a pharmaceutically acceptable salt thereof, wherein Ring B is 1,2,3,6-tetrahydropyridinyl or piperidinyl, each optionally substituted with one or more R′ independently selected from oxo, alkyl, alkynyl, heteroalkyl, or cyano.

17. The compound of any one of claims 1-13, or a pharmaceutically acceptable salt thereof, wherein Ring B is aryl optionally substituted with one or more R′.

18. The compound of claim 17, or a pharmaceutically acceptable salt thereof, wherein Ring B is phenyl optionally substituted with one or more R′.

19. The compound of any one of claims 1-13, or a pharmaceutically acceptable salt thereof, wherein Ring B is heteroaryl optionally substituted with one or more R′.

20. The compound of claim 19, or a pharmaceutically acceptable salt thereof, wherein Ring B is pyridinyl or pyrimidinyl, each optionally substituted with one or more R′.

21. The compound of any one of preceding claims, or a pharmaceutically acceptable salt thereof, wherein each R′ is independently halogen.

22. The compound of any one of preceding claims, or a pharmaceutically acceptable salt thereof, wherein Ring Q is cycloalkyl.

23. The compound of any one of claims 1-21, or a pharmaceutically acceptable salt thereof, wherein Ring Q is heterocyclyl.

24. The compound of any one of claims 1-21, or a pharmaceutically acceptable salt thereof, wherein Ring Q is aryl.

25. The compound of claim 24, or a pharmaceutically acceptable salt thereof, wherein Ring Q is phenyl, naphthalenyl or tetrahydronaphthalenyl.

26. The compound of any one of claims 1-21, or a pharmaceutically acceptable salt thereof, wherein Ring Q is heteroaryl.

27. The compound of claim 26, or a pharmaceutically acceptable salt thereof, wherein Ring Q is selected from pyridinyl, benzothiophenyl, benzoimidazolyl, quinazolinyl, benzotriazolyl, thiophenyl, thienopyridinyl, isoquinolinyl, indolyl, or indazolyl.

28. The compound of claim 1, or a pharmaceutically acceptable salt thereof, wherein G¹ is —[C(R^{d})₂]ᵤ—.

29. The compound of claim 1, or a pharmaceutically acceptable salt thereof, wherein G¹ is —O—.

30. The compound of claim 1, or a pharmaceutically acceptable salt thereof, wherein G¹ is —N(R^{c})—.

31. The compound of claim 30, or a pharmaceutically acceptable salt thereof, wherein R^{c} is hydrogen or alkyl.

32. The compound of claim 1, or a pharmaceutically acceptable salt thereof, wherein G¹ is —C(R^{d})═C(R^{d})—.

33. The compound of claim 32, or a pharmaceutically acceptable salt thereof, wherein R^{d} is hydrogen or alkyl.

34. The compound of claim 1, or a pharmaceutically acceptable salt thereof, wherein G² is a bond.

35. The compound of claim 1, or a pharmaceutically acceptable salt thereof, wherein G² is —[C(R^{d})₂]ᵤ—.

36. The compound of claim 35, or a pharmaceutically acceptable salt thereof, wherein each R^{d} is independently hydrogen or alkyl optionally substituted with one or more deuterium.

37. The compound of claim 1, or a pharmaceutically acceptable salt thereof, wherein G² is —N(R^{c})—.

38. The compound of claim 37, or a pharmaceutically acceptable salt thereof, wherein R^{c} is hydrogen or alkyl optionally substituted with one or more deuterium.

39. The compound of claim 1, or a pharmaceutically acceptable salt thereof, wherein G² is —C(O)—.

40. The compound of claim 1, or a pharmaceutically acceptable salt thereof, wherein G² is —C(O)C(R^{d})₂—.

41. The compound of claim 40, or a pharmaceutically acceptable salt thereof, wherein each R^{d} is independently hydrogen or alkyl, wherein the alkyl is optionally substituted with one or more deuterium.

42. The compound of claim 1, or a pharmaceutically acceptable salt thereof, wherein G¹ is —[C(R^{d})₂]ᵤ— or —O—, and G² is —[C(R^{d})₂]ᵤ— or —N(R^{c})—.

43. The compound of claim 42, or a pharmaceutically acceptable salt thereof, wherein G¹ is —[C(R^{d})₂]ᵤ— and G² is —N(R^{c})—.

44. The compound of claim 43, or a pharmaceutically acceptable salt thereof, wherein -G¹-G²- is —CH₂—N(CH₃)—.

45. The compound of claim 42, or a pharmaceutically acceptable salt thereof, wherein G¹ is —O— and G² is —[C(R^{d})₂]ᵤ—.

46. The compound of claim 45, or a pharmaceutically acceptable salt thereof, wherein -G¹-G²- is —O—CH₂—, —O—CH(CH₃)— or —O—CH(CD₃)—.

47. The compound of any one of preceding claims, or a pharmaceutically acceptable salt thereof, wherein m is 0.

48. The compound of any one of claims 1-46, or a pharmaceutically acceptable salt thereof, wherein m is an integer from 1 to 3, and each R¹ is independently halogen, alkyl, alkenyl or alkylalkoxy, wherein the alkyl, alkenyl and alkylalkoxy are optionally substituted with one or more deuterium.

49. The compound of any one of claims 1-46, or a pharmaceutically acceptable salt thereof, wherein m is 1, and R¹ is —CH₃, —CD₃, —CH═CH₂, —CH₂—O—CH₃, —CH₂—O-CD₃, —C(O)R* or —C(O)OR*, wherein R* is alkyl or alkylaryl.

50. The compound of any one of claims 1-49, or a pharmaceutically acceptable salt thereof, wherein 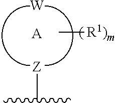
is selected from the group consisting of:

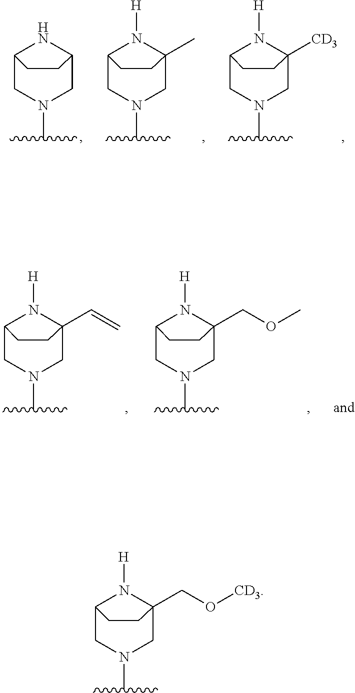

51. The compound of any one of preceding claims, or a pharmaceutically acceptable salt thereof, wherein n is an integer from 1 to 4, and each R² is independently selected from hydroxyl, halogen, cyano, amino, alkyl, alkenyl, alkynyl, haloalkyl, or cycloalkyl, wherein alkyl, alkenyl, alkynyl, haloalkyl, and cycloalkyl are optionally substituted with one or more groups independently selected from cyano, hydroxyl, halogen, or alkyl.

52. The compound of claim 1, or a pharmaceutically acceptable salt thereof, wherein L is

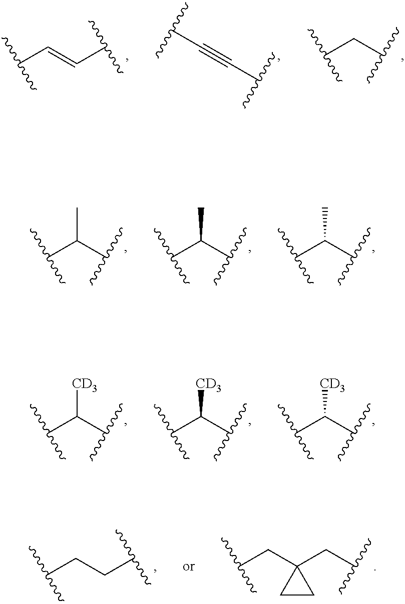

53. The compound of any one of preceding claims, or a pharmaceutically acceptable salt thereof, wherein Ring E is heterocyclyl.

54. The compound of claim 53, or a pharmaceutically acceptable salt thereof, wherein Ring E is selected from the group consisting of:

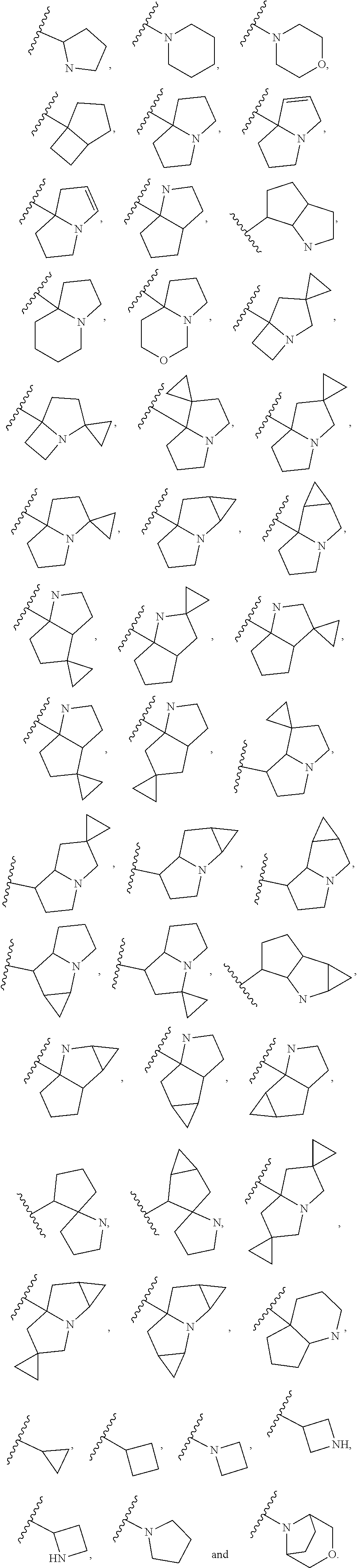

55. The compound of any one of preceding claims, or a pharmaceutically acceptable salt thereof, wherein R³ is selected from hydrogen, halogen, alkyl, alkoxy, hydroxyalkyl, heterocyclyl, —N(R^{g})₂ or ═C(R″)₂, wherein the alkyl, alkoxy, hydroxyalkyl, and heterocyclyl are optionally substituted with one or more groups selected from deuterium.

56. The compound of claim 55, or a pharmaceutically acceptable salt thereof, wherein each R″ is independently selected from hydrogen, hydroxyl, halogen, or alkyl.

57. The compound of any one of claims 1-56, or a pharmaceutically acceptable salt thereof, wherein v is an integer from 1 to 4, each R³ is independently selected from F, —CH₃, —CD₃, ═CH₂, ═CHF, ═CF₂, —CH₂OH, —OCH₃, —N(CH₃)₂,

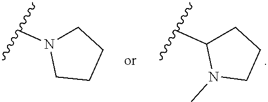

58. The compound of any one of preceding claims, or a pharmaceutically acceptable salt thereof, wherein v is an integer from 1 to 4, at least one R³ is ═CH₂, ═CHF, or ═CF₂.

59. The compound of any one of claims 55-58, or a pharmaceutically acceptable salt thereof, wherein v is an integer from 2 to 4, one R³ is ═C(R″)₂, and other R³ is independently selected from hydroxyl, halogen, alkyl, alkoxy, or ═C(R″)₂.

60. The compound of any one of preceding claims, or a pharmaceutically acceptable salt thereof, wherein 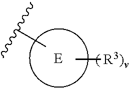
is selected from the group consisting of:

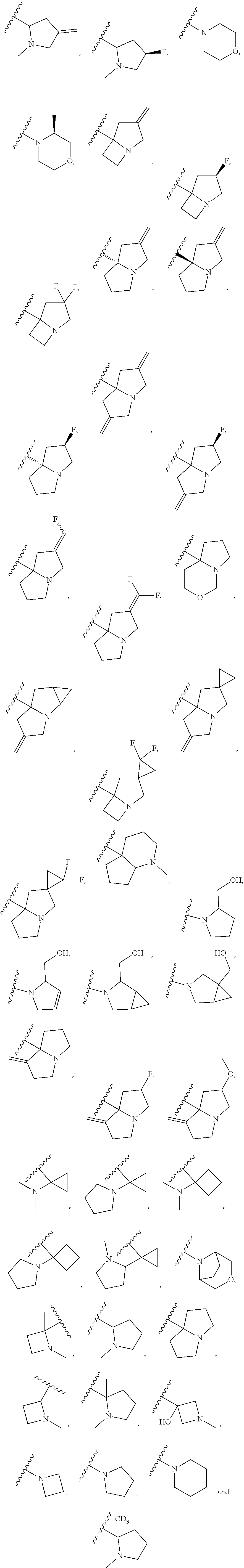

61. The compound of claim 1, or a pharmaceutically acceptable salt thereof, wherein Ring E is cycloalkyl.

62. The compound of claim 61, or a pharmaceutically acceptable salt thereof, wherein Ring E is selected from cyclopropyl, cyclobutyl, cyclopentyl, or cyclohexyl.

63. The compound of claim 1, or a pharmaceutically acceptable salt thereof, wherein Ring E is aryl.

64. The compound of claim 63, or a pharmaceutically acceptable salt thereof, wherein Ring E is phenyl or naphthalenyl.

65. The compound of claim 1, or a pharmaceutically acceptable salt thereof, wherein Ring E is heteroaryl.

66. The compound of claim 65, or a pharmaceutically acceptable salt thereof, wherein Ring E is selected from the group consisting of pyridinyl, pyrimidinyl pyridazinyl, pyrazinyl, thienyl, thiazolyl, isothiazolyl, oxazolyl, isoxazolyl, imidazolyl, pyrazolyl, 1,2,3-triazolyl, 1,2,4-triazolyl, benzofuranyl, benzothienyl, indolyl, benzimidazolyl, benzopyrazolyl, purinyl, quinolinyl, isoquinolinyl, isoquinoline-1(2H)-one group, isoindolin-1-one group, benzo[d]oxazole-2(H)-one group and 1,3-dihydro-2H-benzo[d]imidazol-2-one group.

67. The compound of any one of preceding claims, or a pharmaceutically acceptable salt thereof, wherein T is N.

68. The compound of any one of claims 1-68, or a pharmaceutically acceptable salt thereof, wherein T is C(R^{T}).

69. The compound of claim 70, or a pharmaceutically acceptable salt thereof, wherein R^{T} is hydrogen, cyano, fluoro, —CH₃, —OCH₃, or —CF₃.

70. The compound of any one of preceding claims, or a pharmaceutically acceptable salt thereof, wherein Y is O.

71. The compound of any one of claims 1-73, or a pharmaceutically acceptable salt thereof, wherein Y is S.

72. The compound of claim 1, or a pharmaceutically acceptable salt thereof, wherein R⁴ is alkyl optionally substituted with one or more group independently selected from deuterium, hydroxyl, hydroxyalkyl, alkoxy, or —N(R^{h})₂.

73. The compound of claim 72, or a pharmaceutically acceptable salt thereof, wherein R⁴ is selected from

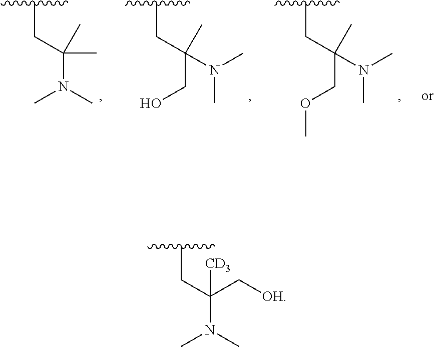

74. The compound of claim 1, or a pharmaceutically acceptable salt thereof, wherein the compound has a formula selected from:

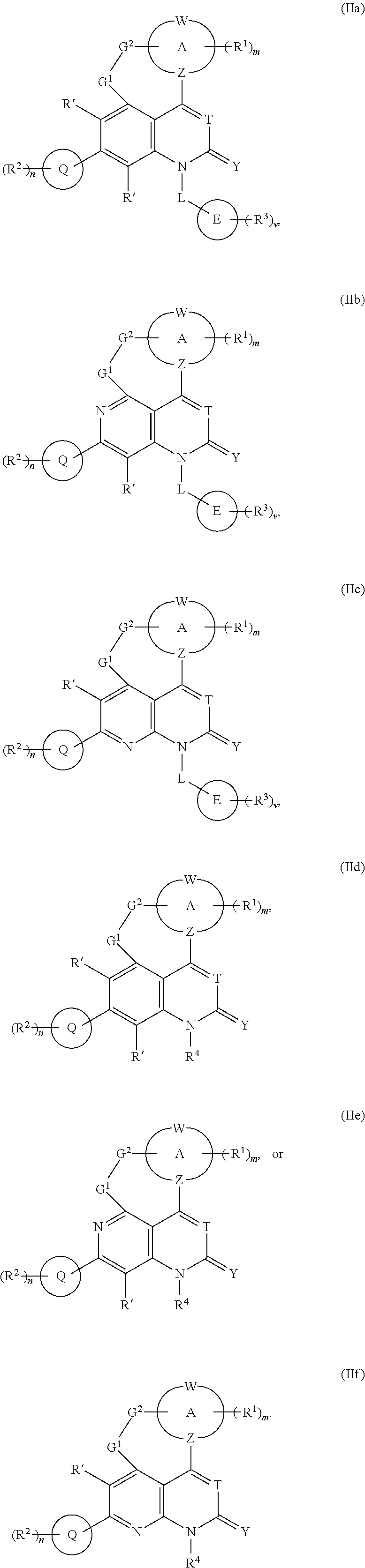

75. The compound of claim 74, or a pharmaceutically acceptable salt thereof, wherein Ring Q is phenyl, naphthalenyl, benzothiophenyl or pyridinyl.

76. The compound of claim 74, or a pharmaceutically acceptable salt thereof, wherein G¹ is —O—.

77. The compound of claim 74, or a pharmaceutically acceptable salt thereof, wherein G² is —[C(R^{d})₂]ᵤ—, and each R^{d} is independently hydrogen or alkyl optionally substituted with one or more deuterium.

78. The compound of claim 74, or a pharmaceutically acceptable salt thereof, wherein G¹ is —O—, G² is —[C(R^{d})₂]ᵤ—, each R^{d} is independently hydrogen or alkyl optionally substituted with one or more deuterium, and u is 1 or 2.

79. The compound of claim 74, or a pharmaceutically acceptable salt thereof, wherein G¹ is —[C(R^{d})₂]ᵤ—, and each R^{d} is independently hydrogen or alkyl optionally substituted with one or more deuterium.

80. The compound of claim 74, or a pharmaceutically acceptable salt thereof, wherein G² is —N(R^{c})—, and R^{c} is hydrogen or alkyl optionally substituted with one or more deuterium.

81. The compound of claim 74, or a pharmaceutically acceptable salt thereof, wherein G¹ is —[C(R^{d})₂]ᵤ—, G² is —N(R^{c})—, each of R^{c} and R^{d} is independently hydrogen or alkyl optionally substituted with one or more deuterium, and u is 1 or 2.

82. The compound of claim 74, or a pharmaceutically acceptable salt thereof, wherein 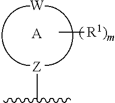
is selected from 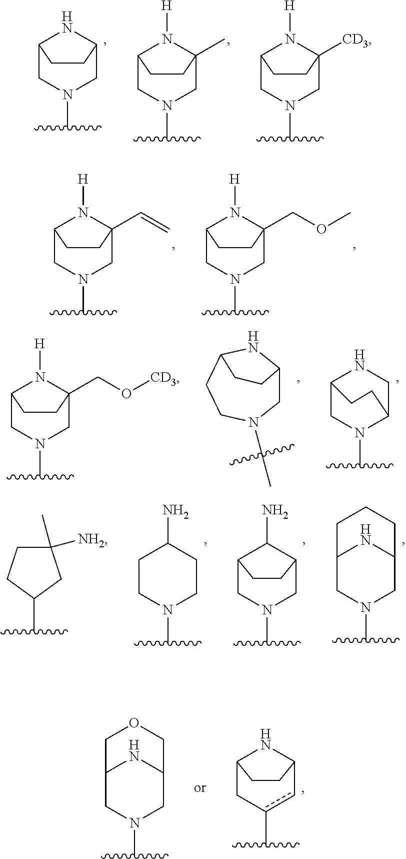
and [symbol] represents a single bond or a double bond.

83. The compound of claim 74, or a pharmaceutically acceptable salt thereof, wherein R³ is selected from hydrogen, halogen, alkyl, alkoxy, hydroxyalkyl, heterocyclyl, —N(R⁸)₂ or ═C(R″)₂, wherein the alkyl, alkoxy, hydroxyalkyl, and heterocyclyl are optionally substituted with one or more groups selected from deuterium.

84. The compound of claim 83, or a pharmaceutically acceptable salt thereof, wherein v is an integer from 1 to 4, each R³ is independently selected from F, —CH₃, —CD₃, ═CH₂, ═CHF, ═CF₂, —CH₂OH, —OCH₃, —N(CH₃)₂,

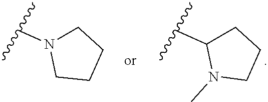

85. The compound of claim 83 or 84, or a pharmaceutically acceptable salt thereof, wherein v is an integer from 1 to 4, at least one R³ is ═CH₂, ═CHF, or ═CF₂.

86. The compound of claim 74, or a pharmaceutically acceptable salt thereof, wherein L is

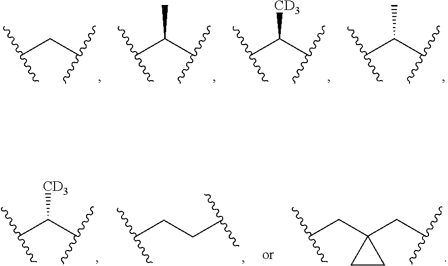

87. The compound of claim 1, or a pharmaceutically acceptable salt thereof, wherein the compound has a formula selected from:

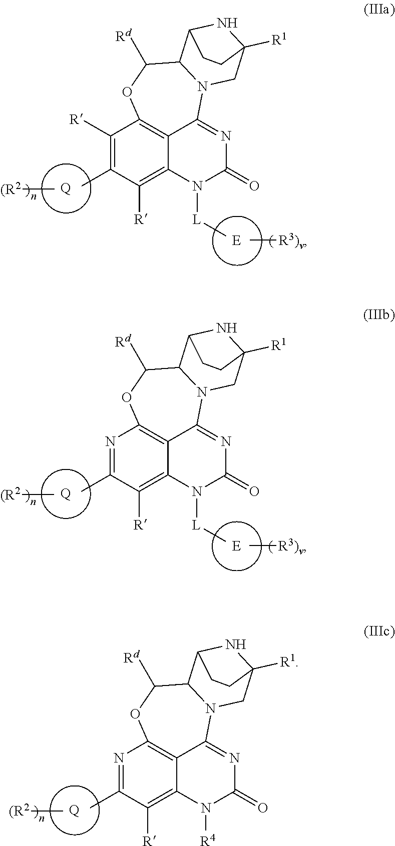

88. The compound of claim 87, or a pharmaceutically acceptable salt thereof, wherein R¹ is independently selected from hydrogen, halogen, alkyl, alkenyl, alkylalkoxy, heteroalkyl, or heteroaryl, and the alkyl, alkenyl, alkylalkoxy, heteroalkyl, and heteroaryl are optionally substituted with one or more deuterium.

89. The compound of claim 88, or a pharmaceutically acceptable salt thereof, wherein R¹ is independently selected from H, —CH₃, —CD₃, —CH═CH₂, —CH₂—O—CH₃, or —CH₂—O-CD₃.

90. The compound of claim 87, or a pharmaceutically acceptable salt thereof, wherein R^{d} is independently hydrogen or alkyl optionally substituted with one or more deuterium.

91. The compound of any one of claims 87-90, or a pharmaceutically acceptable salt thereof, wherein Ring Q is phenyl, naphthalenyl, benzothiophenyl or pyridinyl.

92. The compound of any one of claims 87-91, or a pharmaceutically acceptable salt thereof, wherein 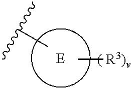
is selected from the group consisting of:

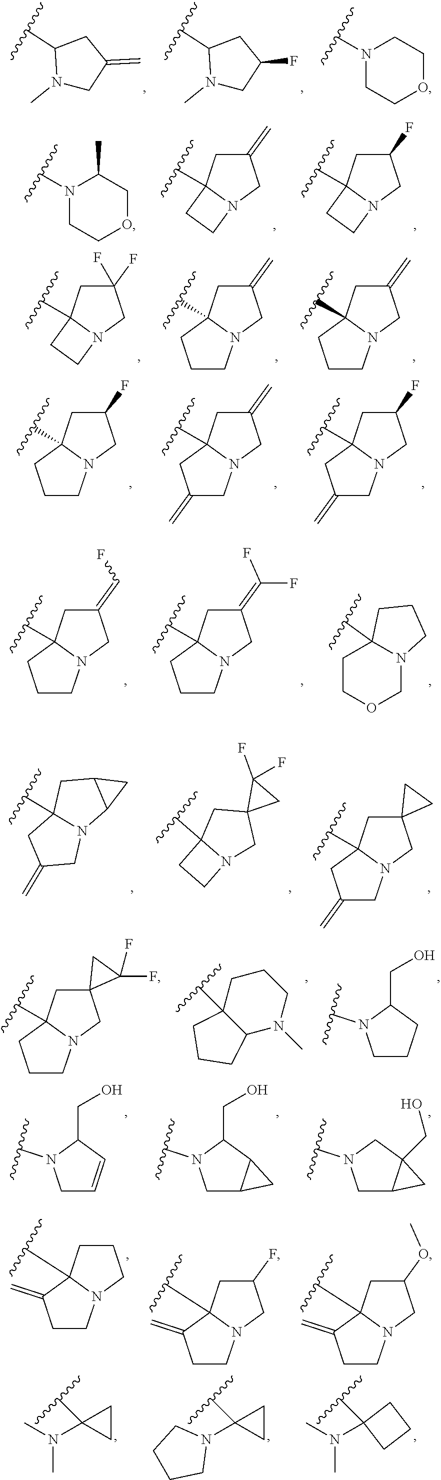

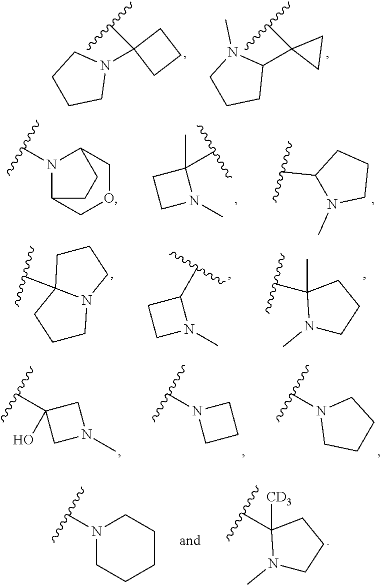

93. A compound selected from the group consisting of: 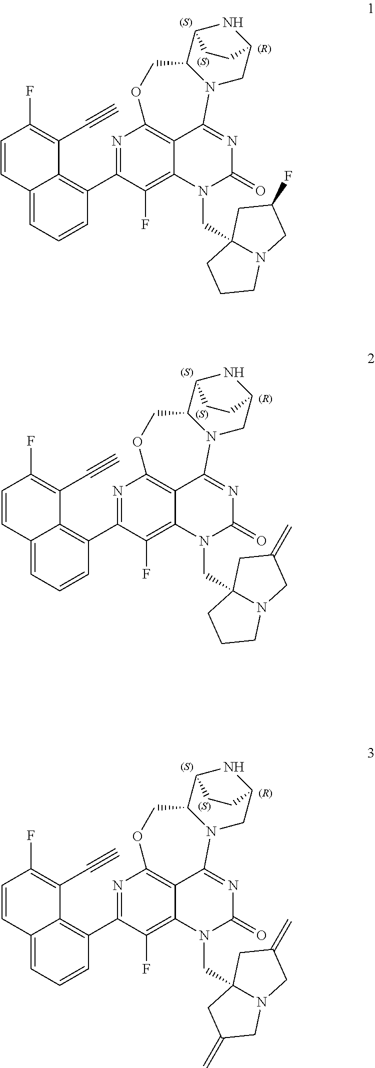 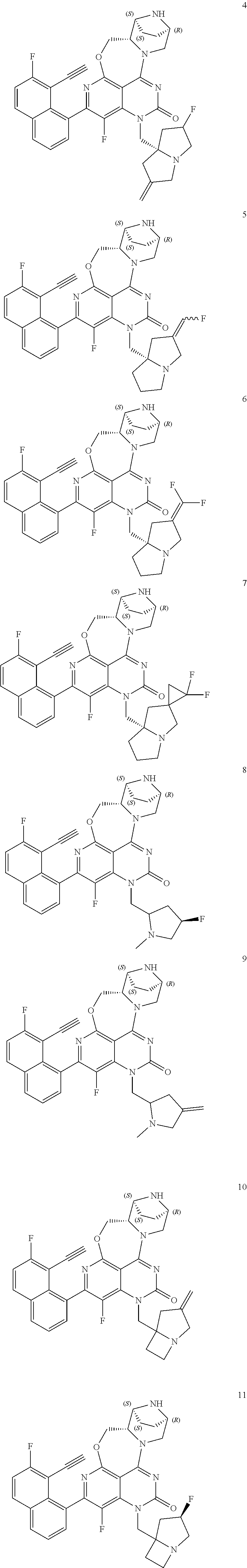 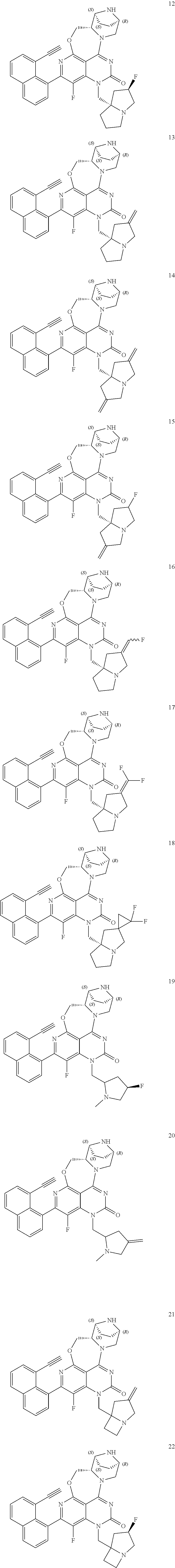 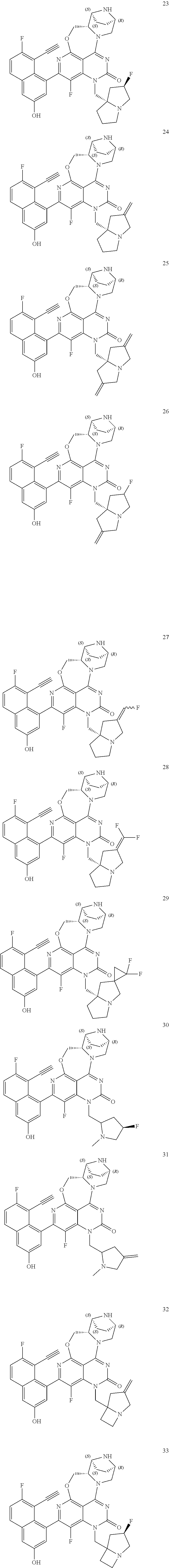 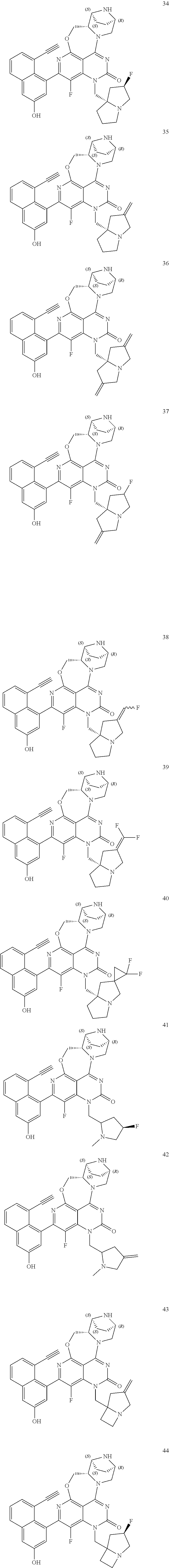 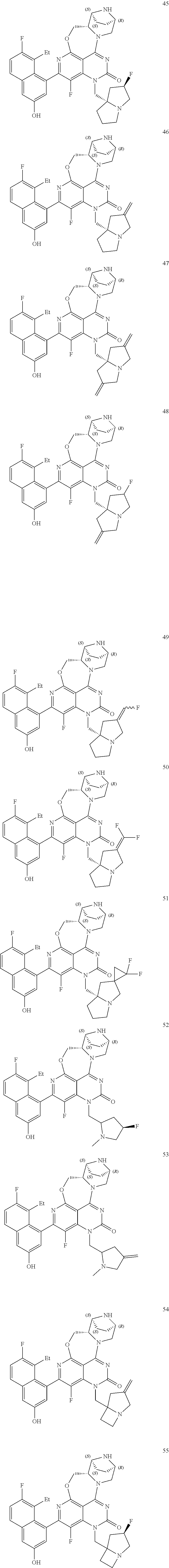 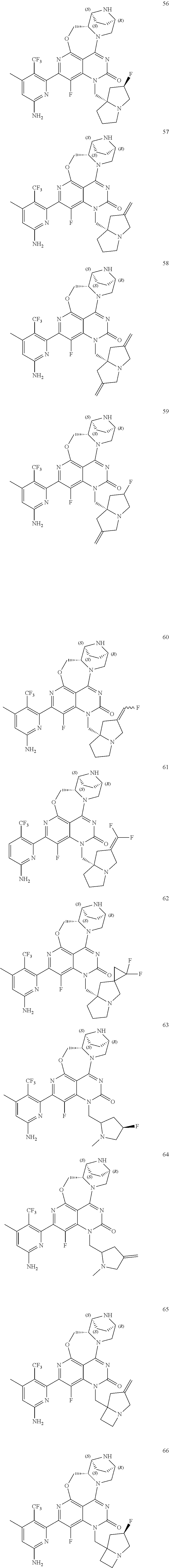 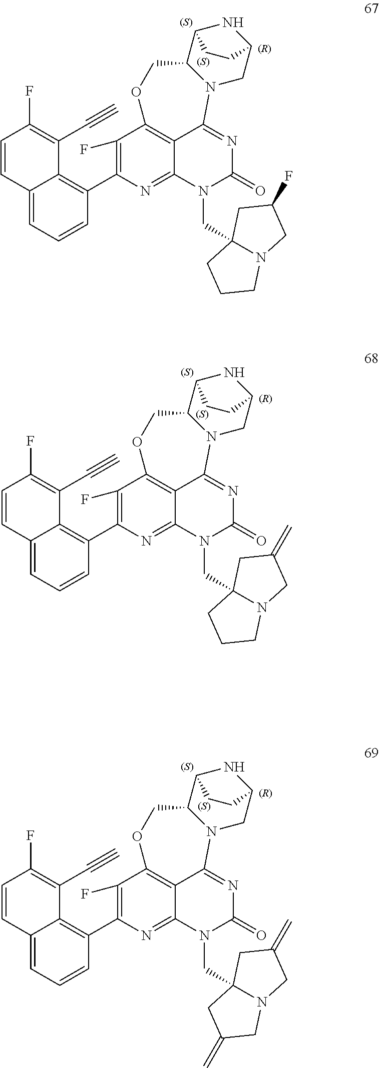 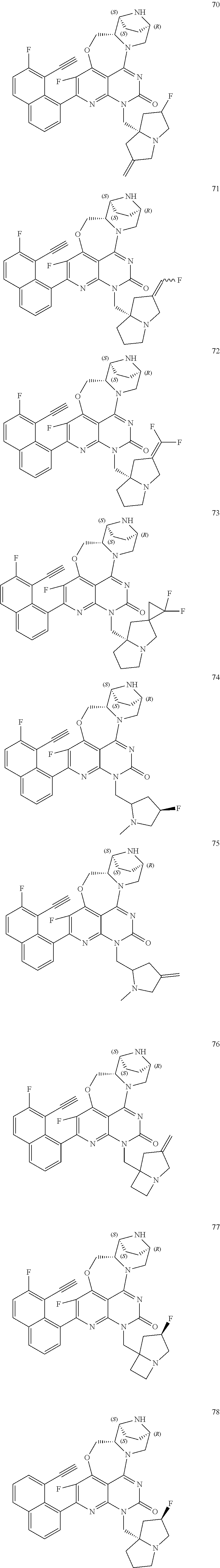 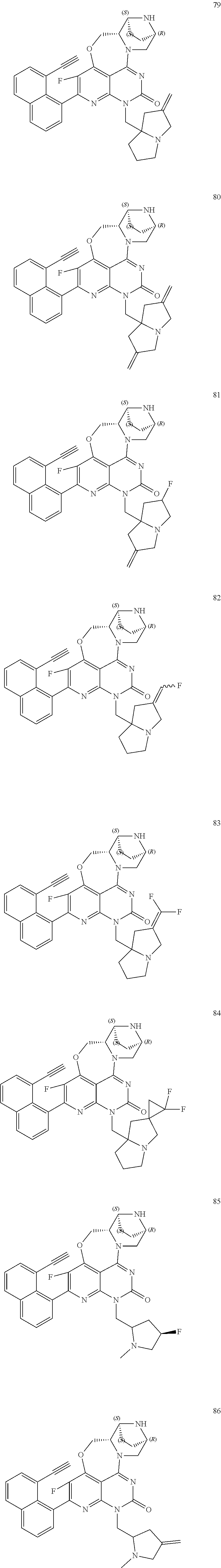 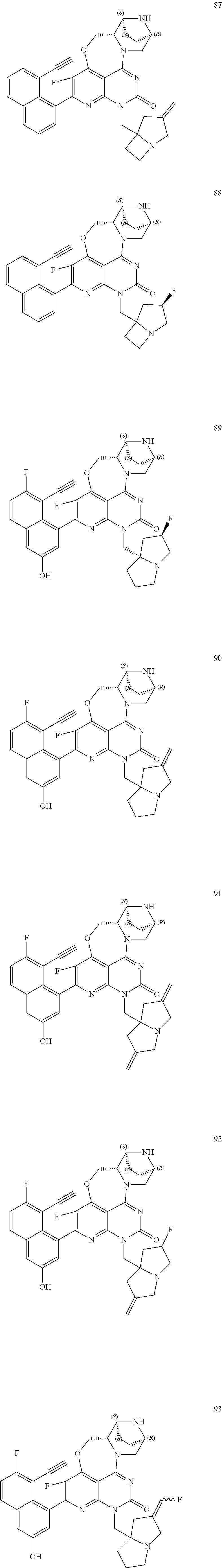 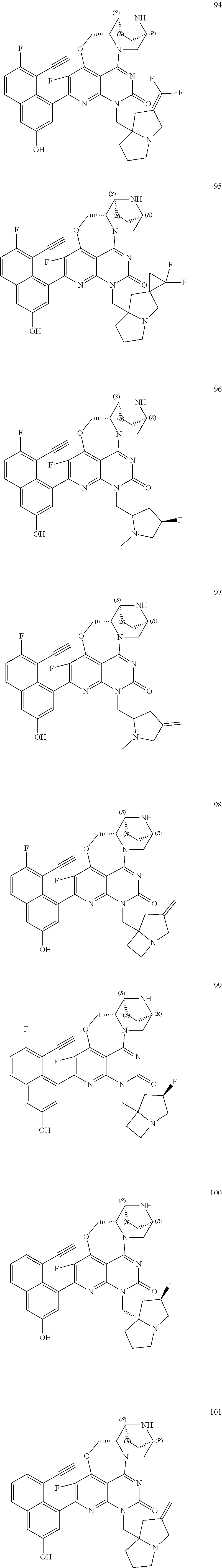 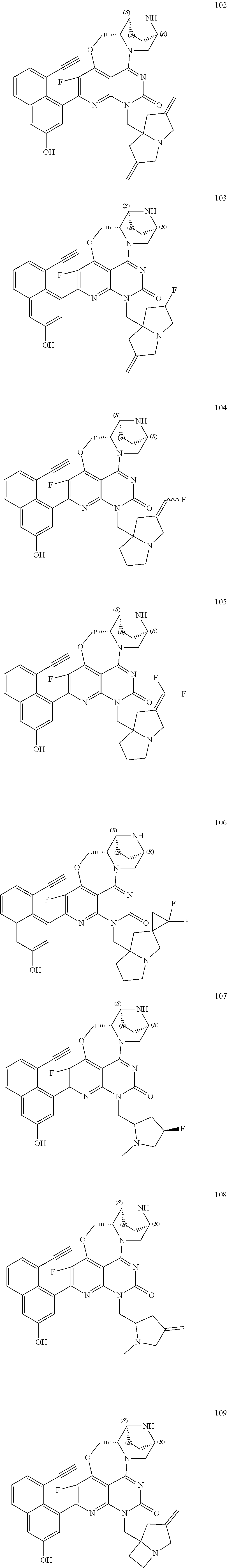 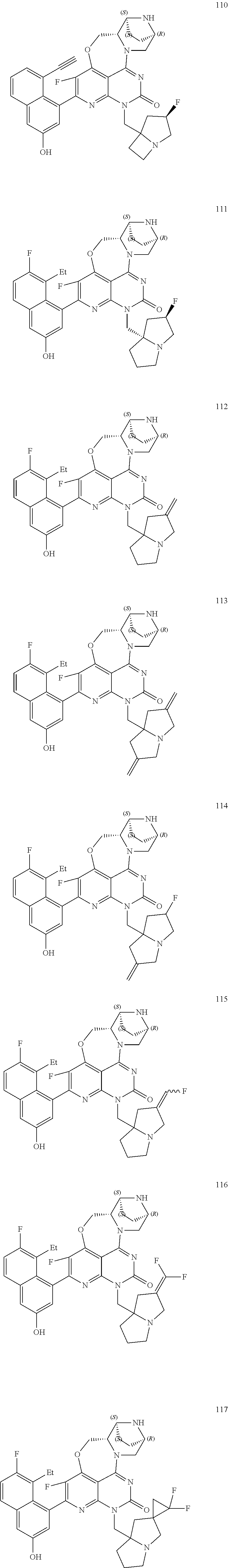 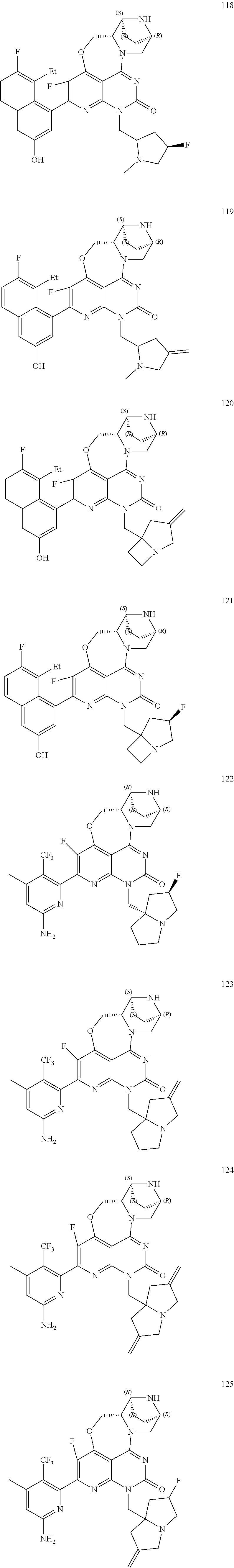 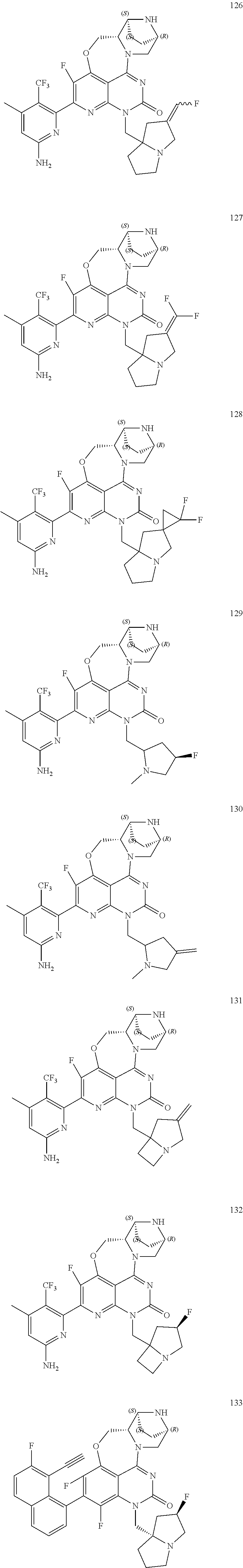 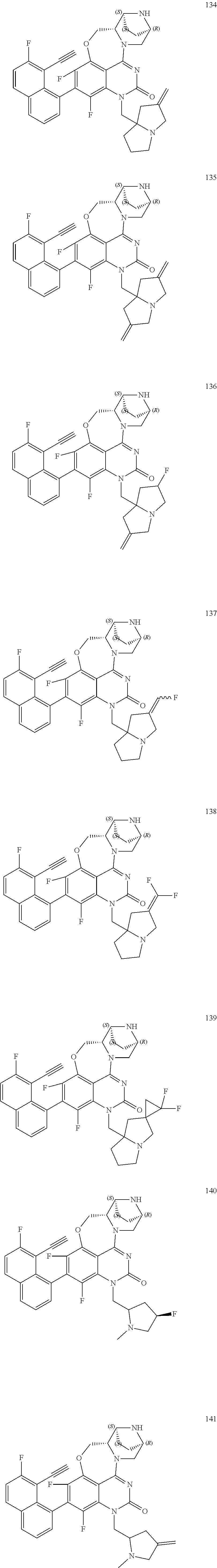 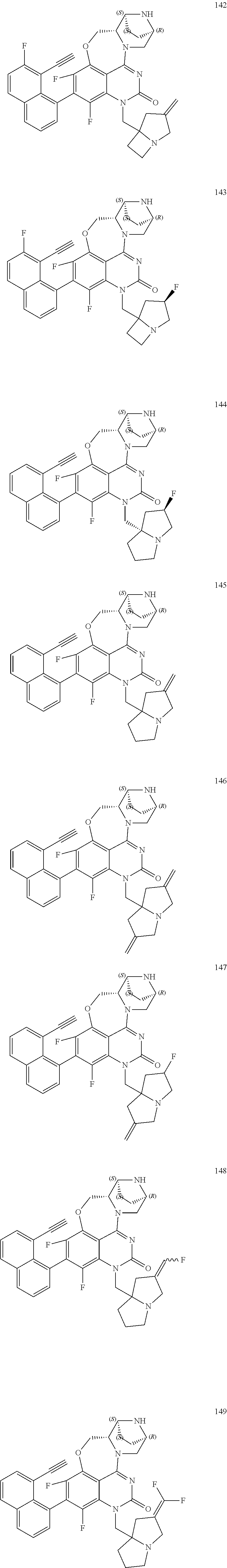 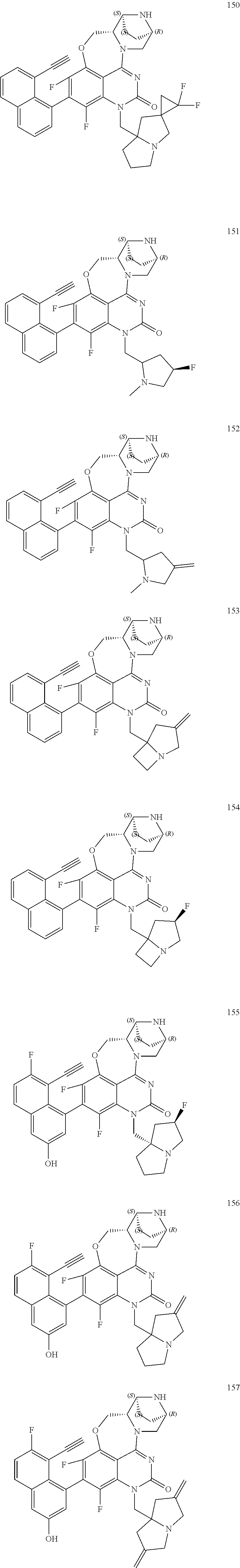 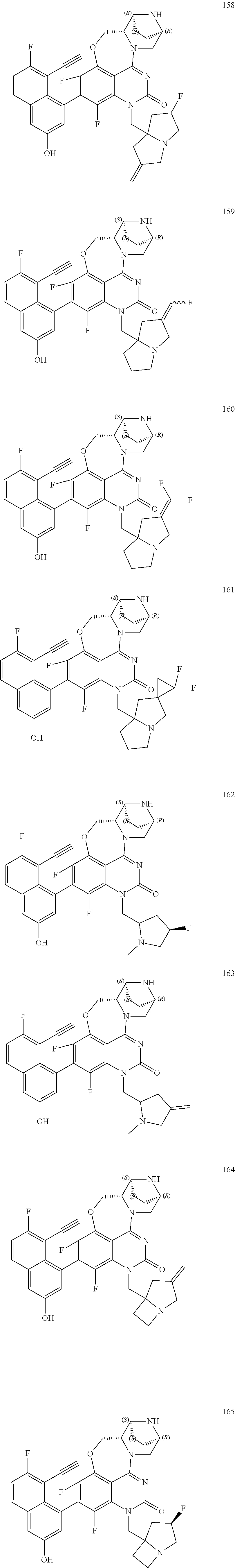 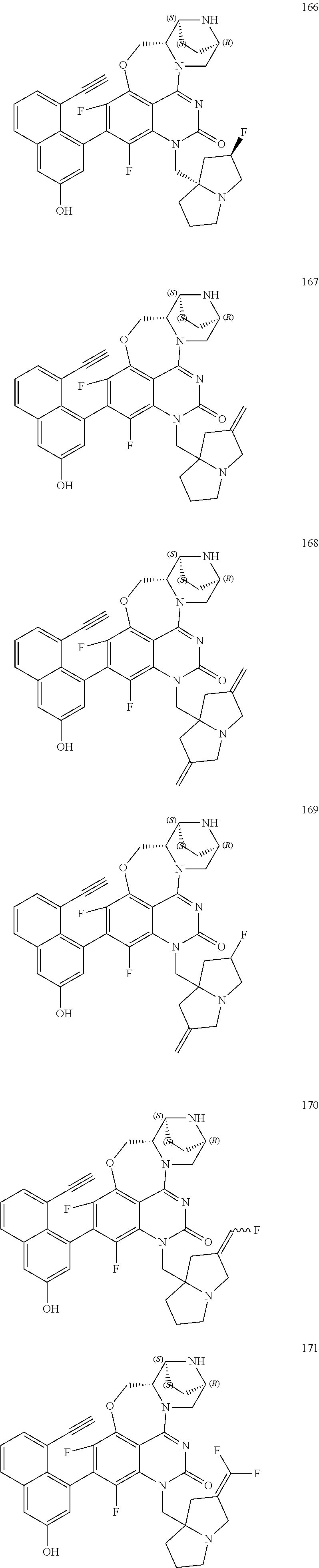 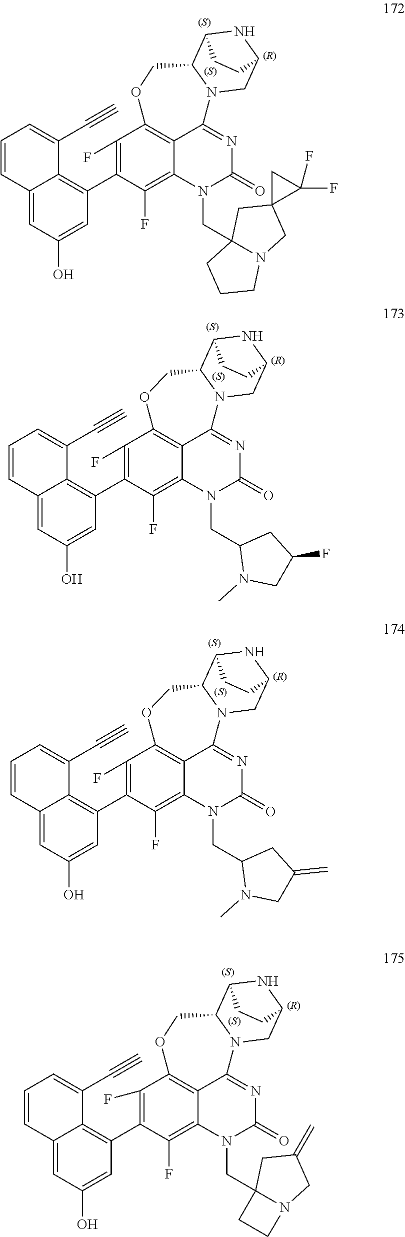 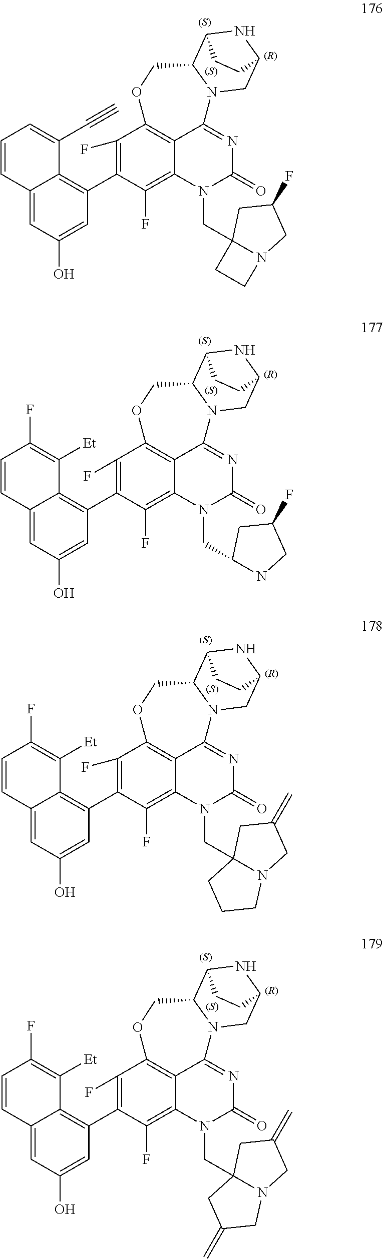 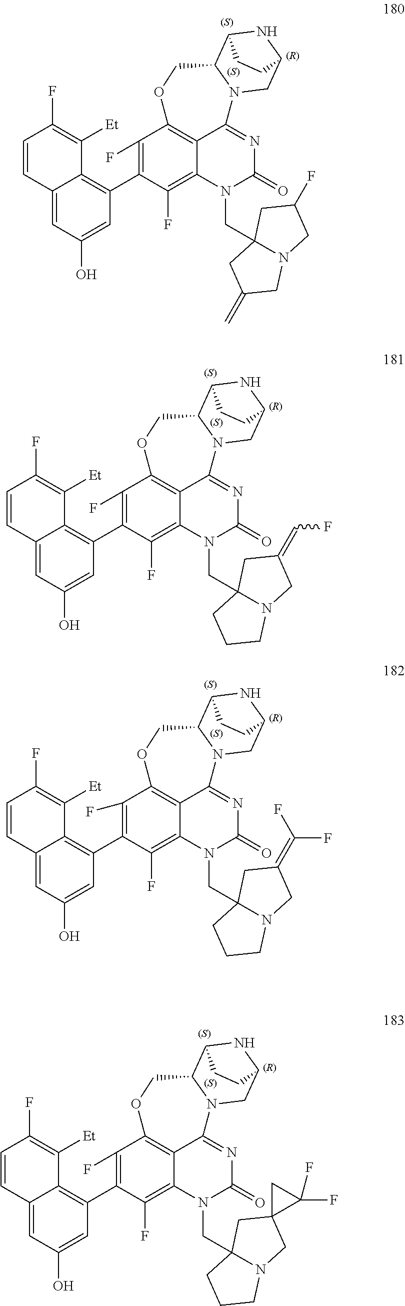 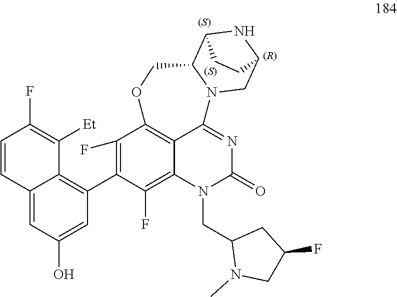 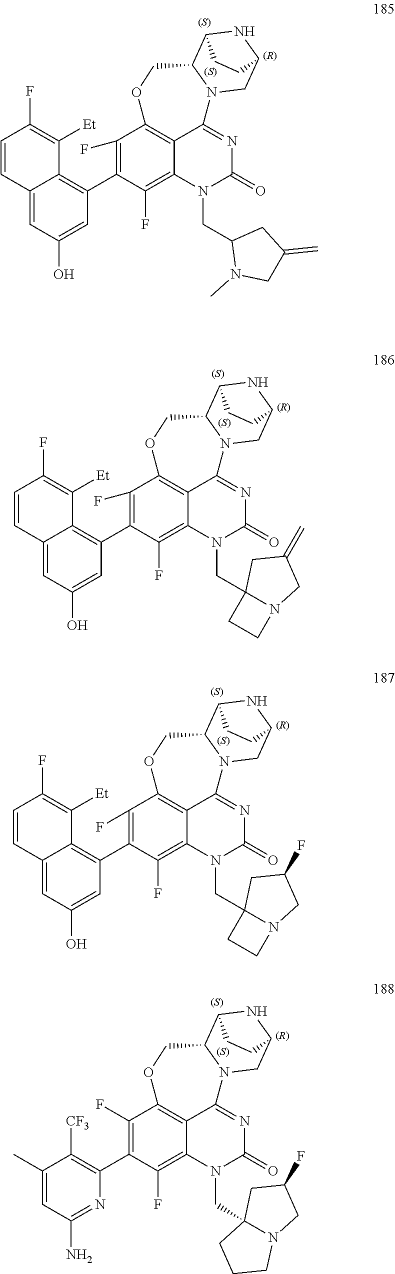 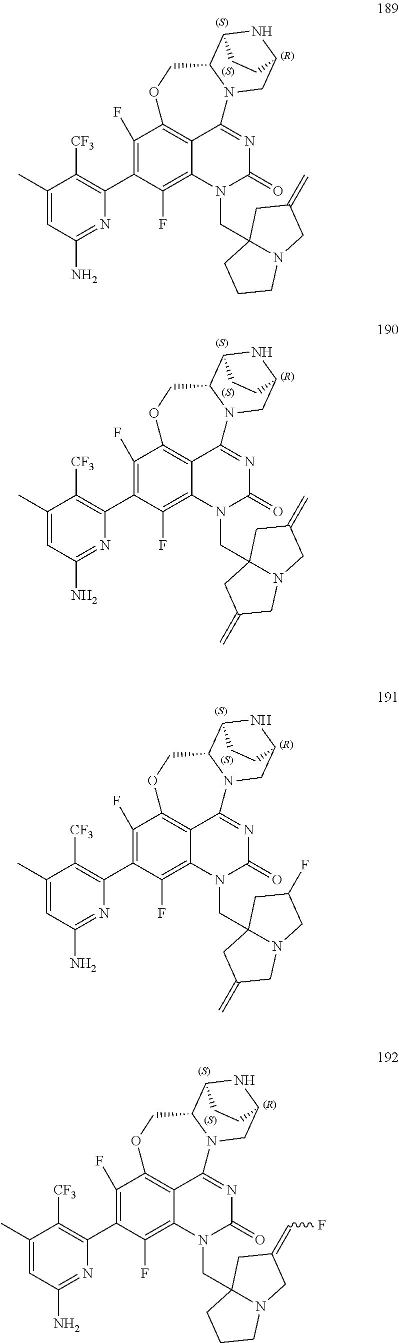 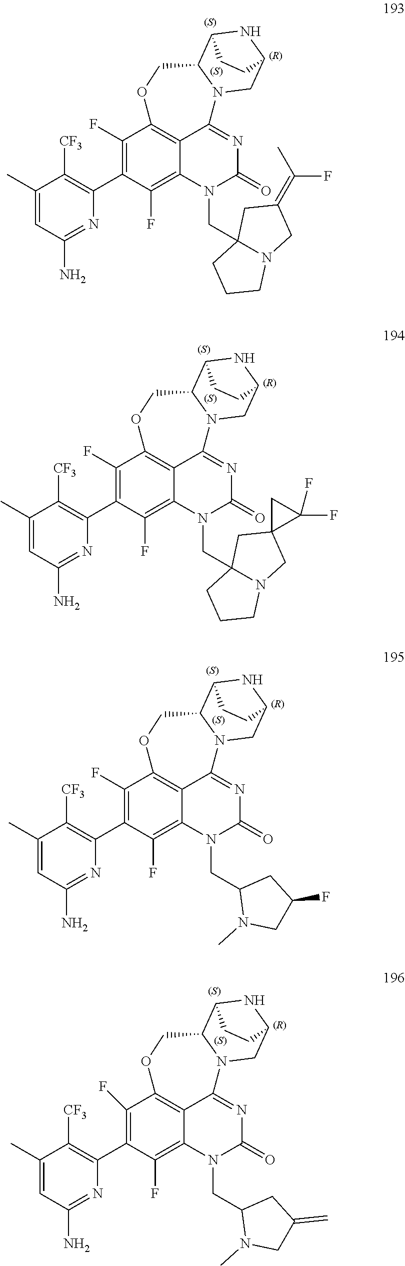 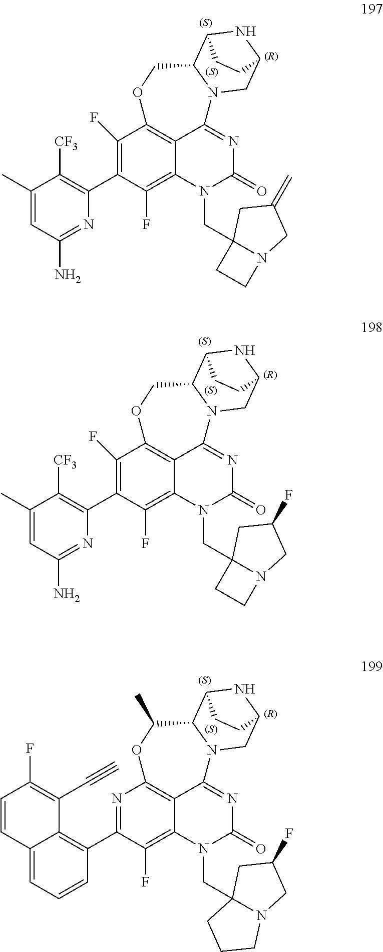 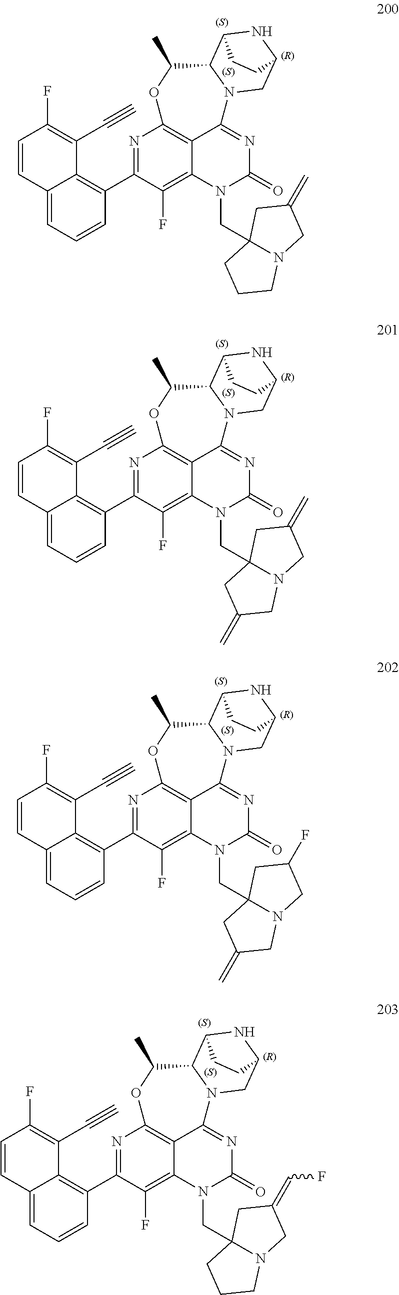 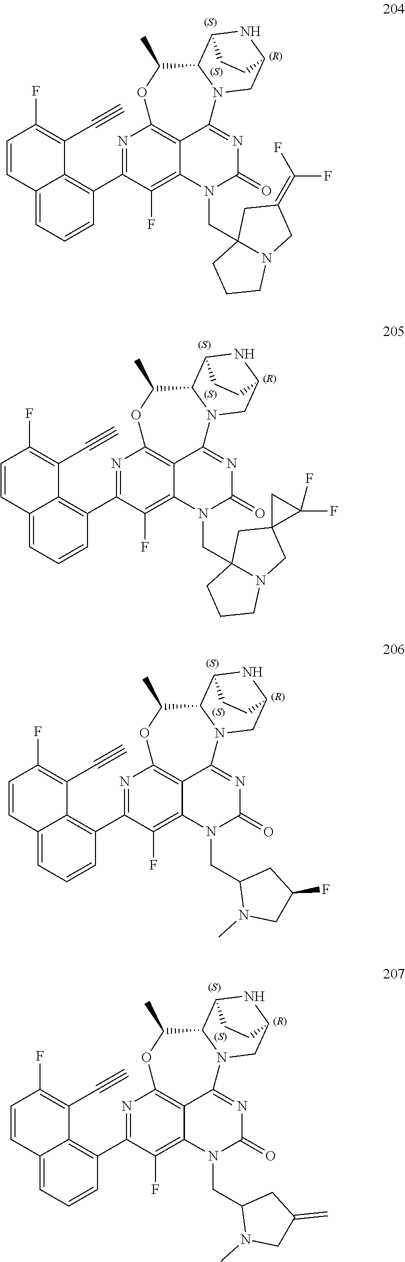 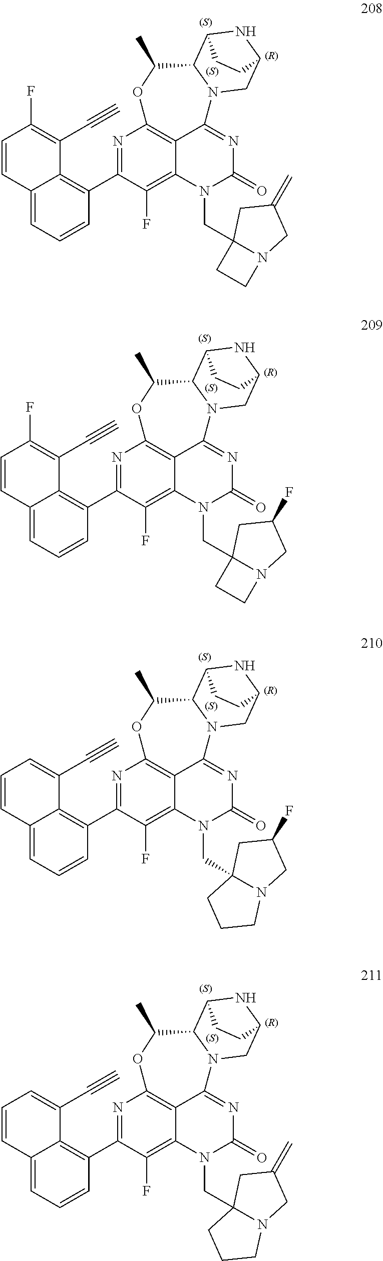 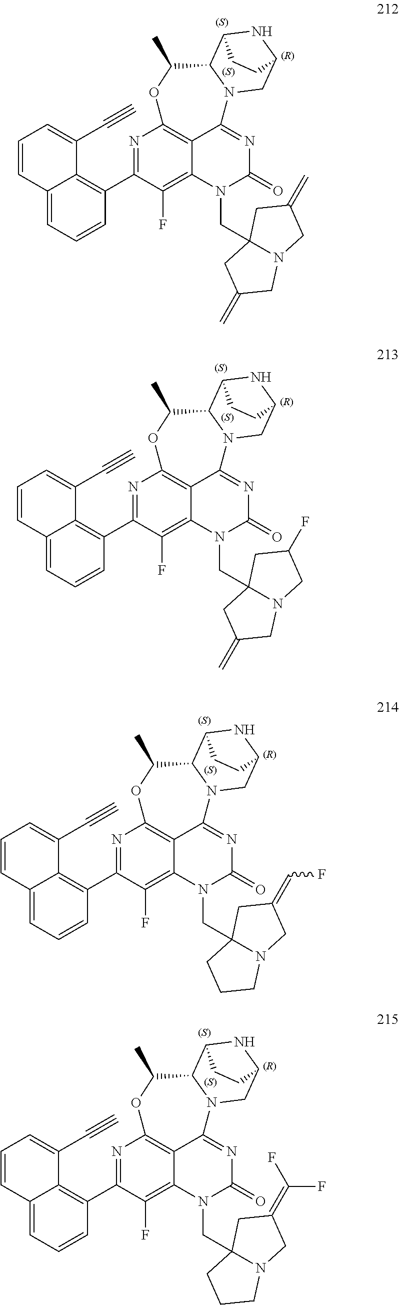 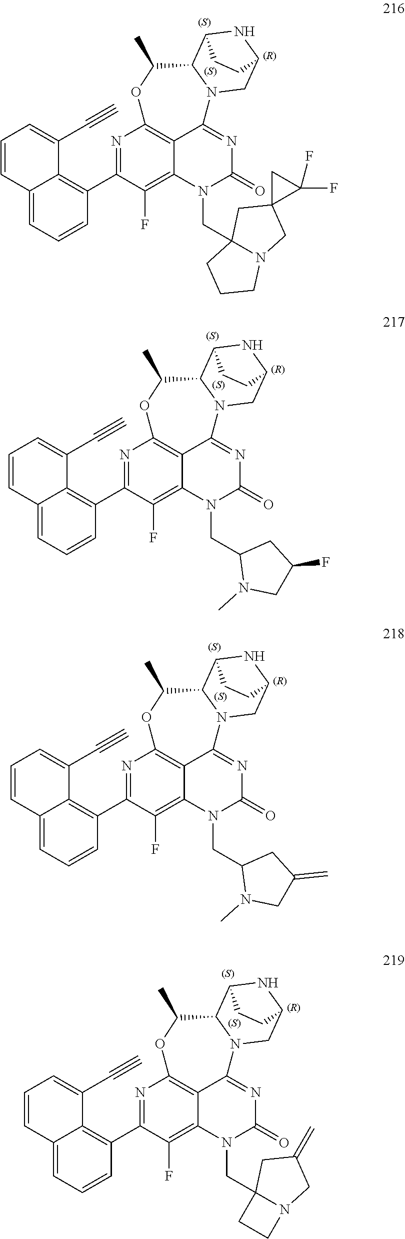 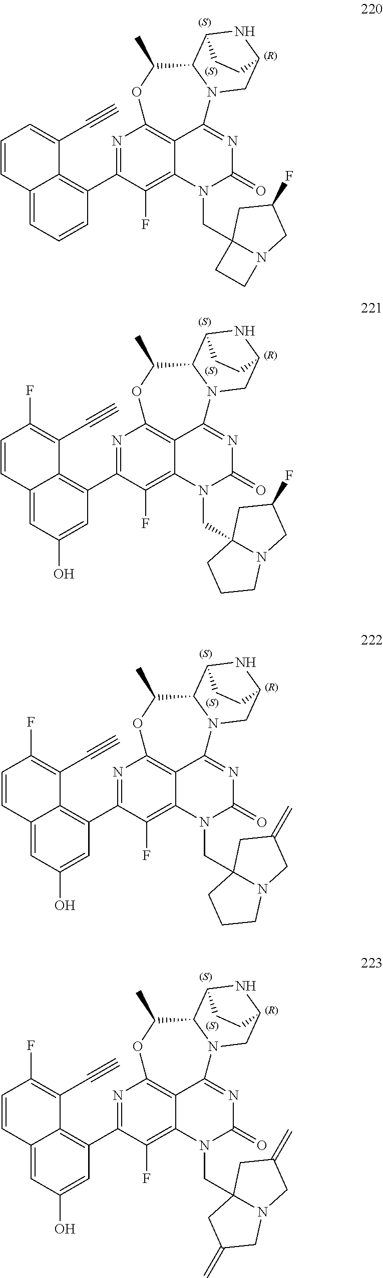 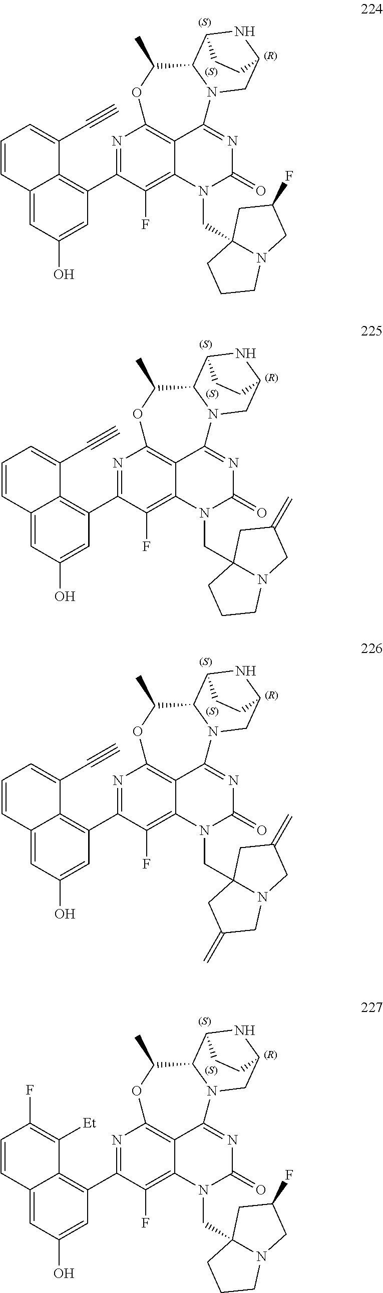 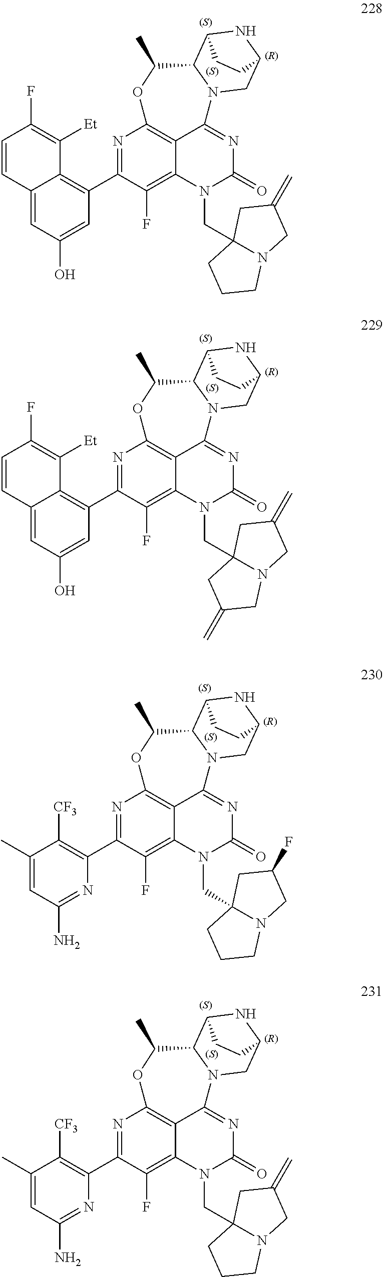 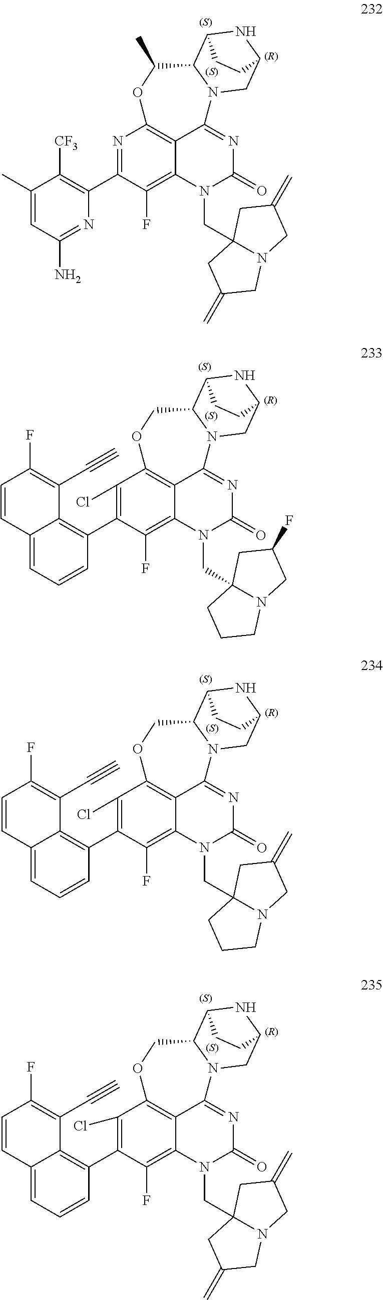 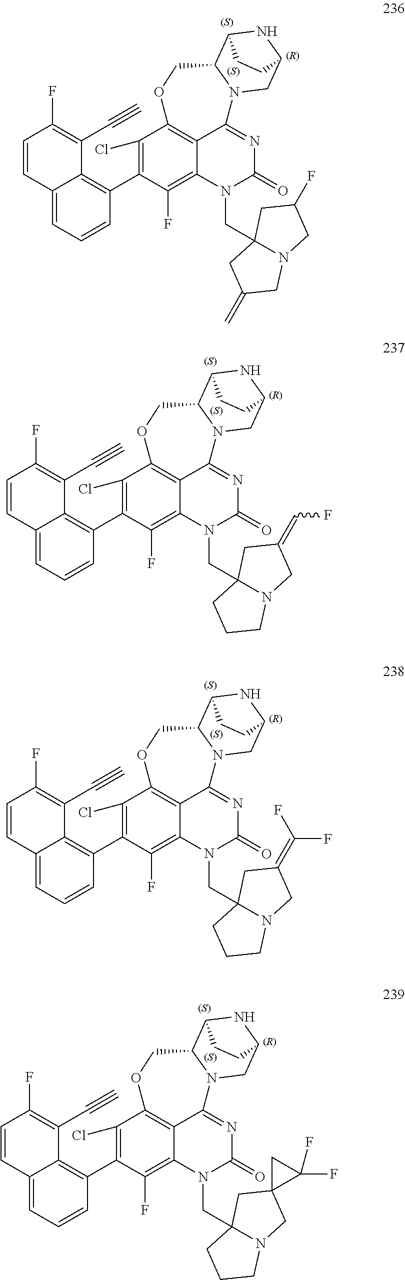 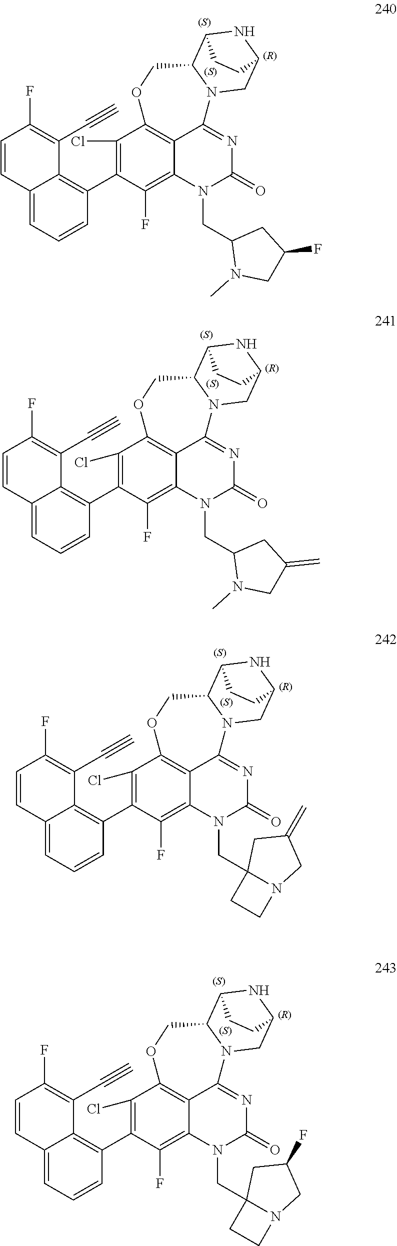 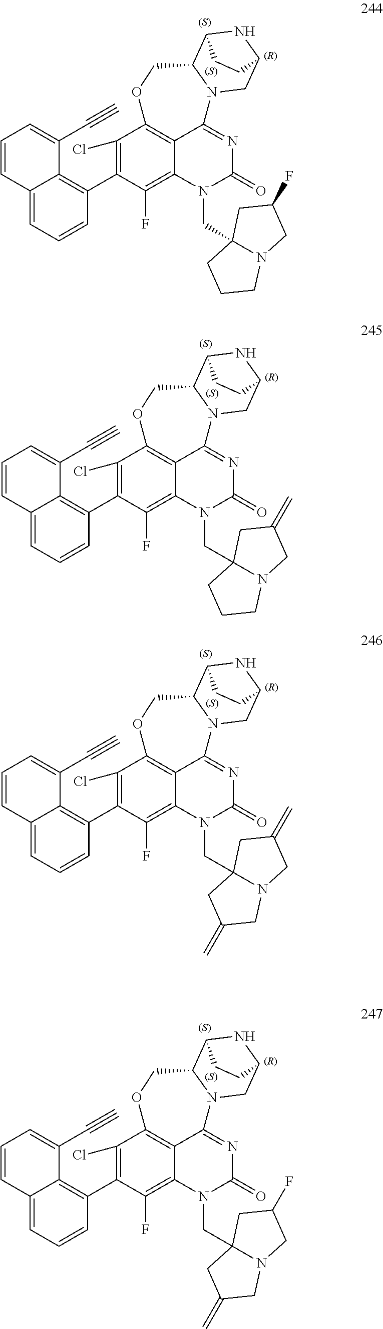 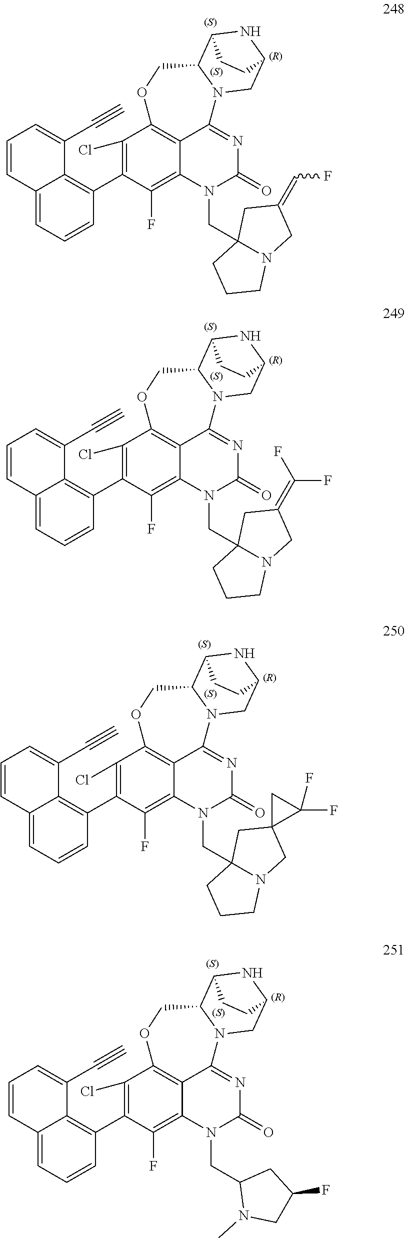 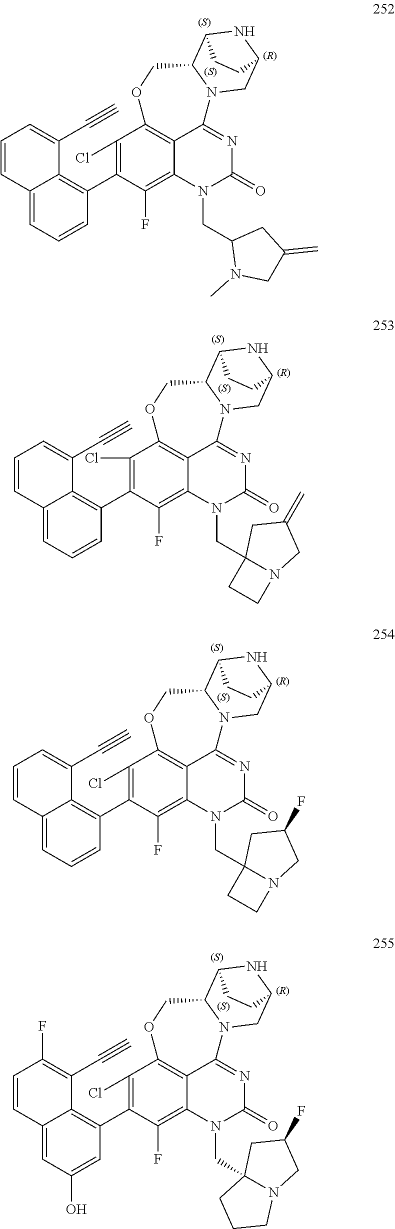 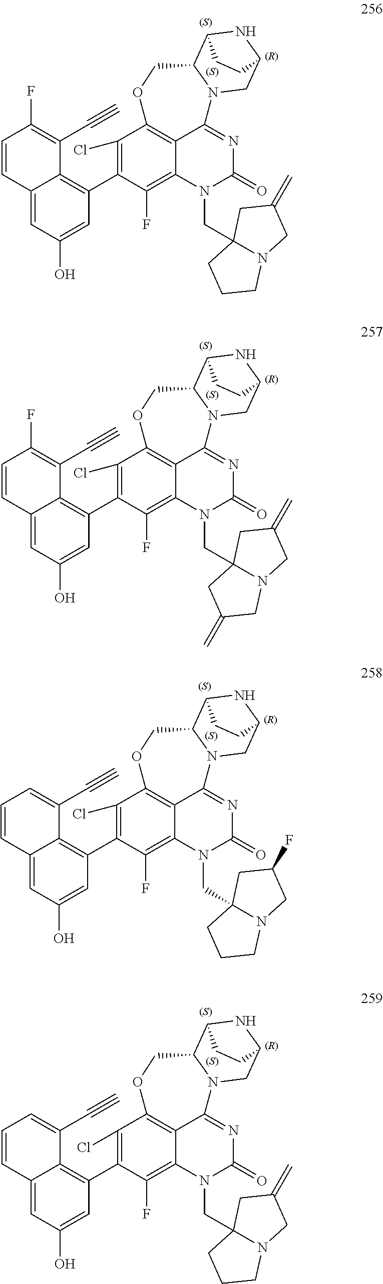 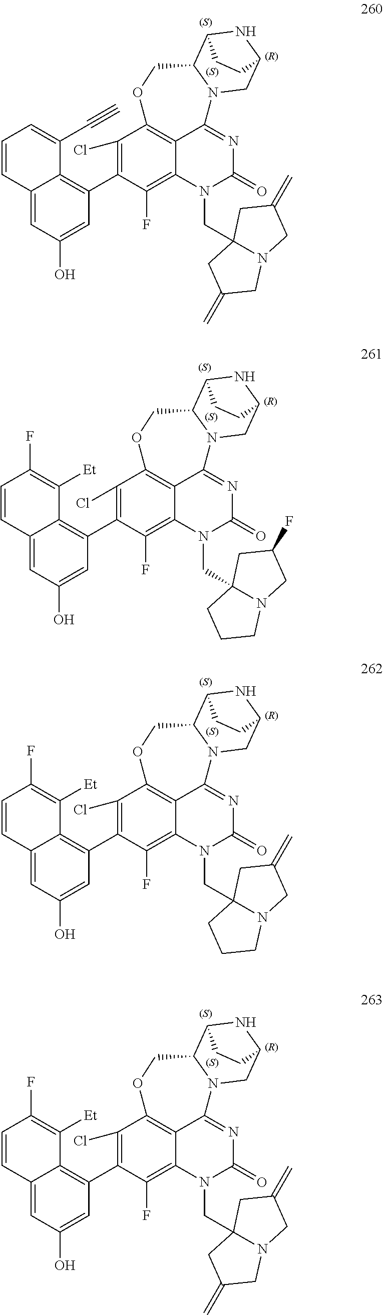 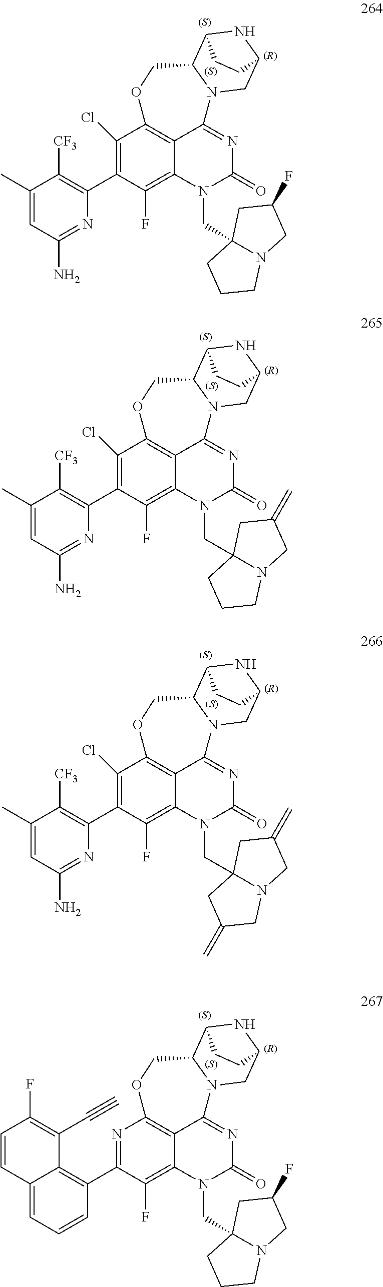 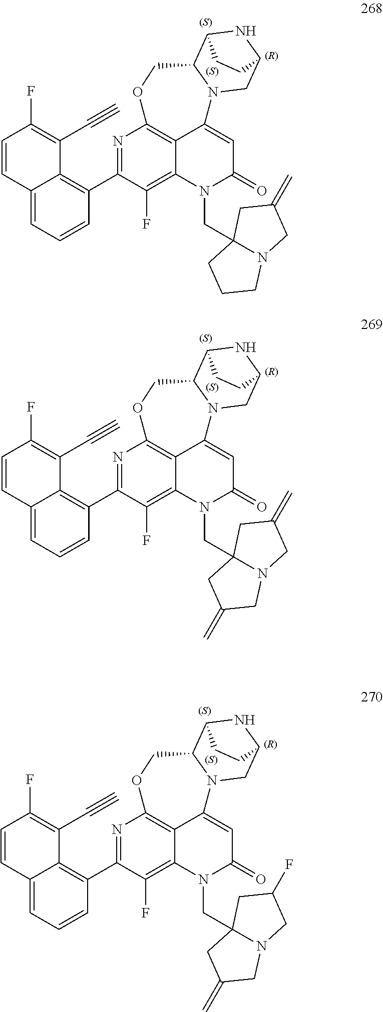 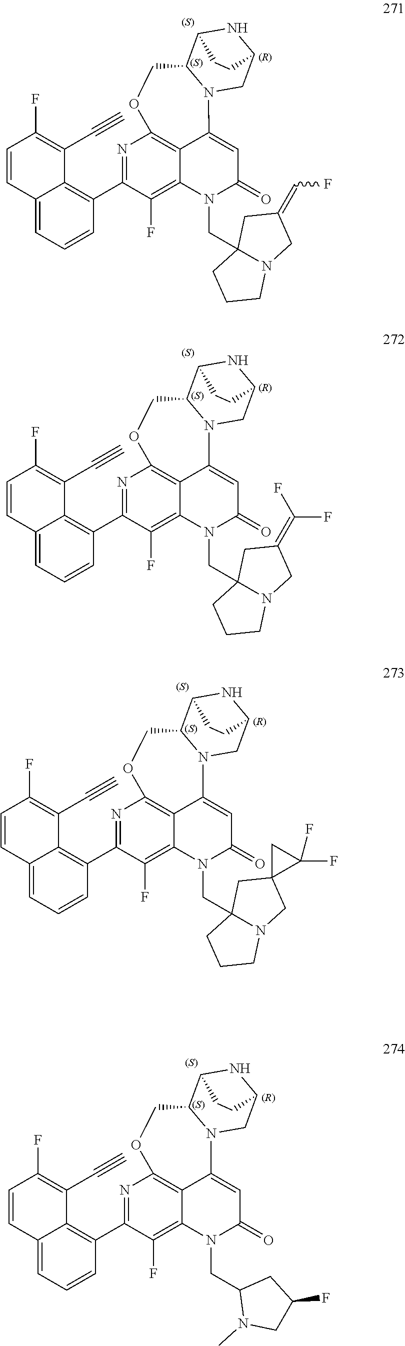 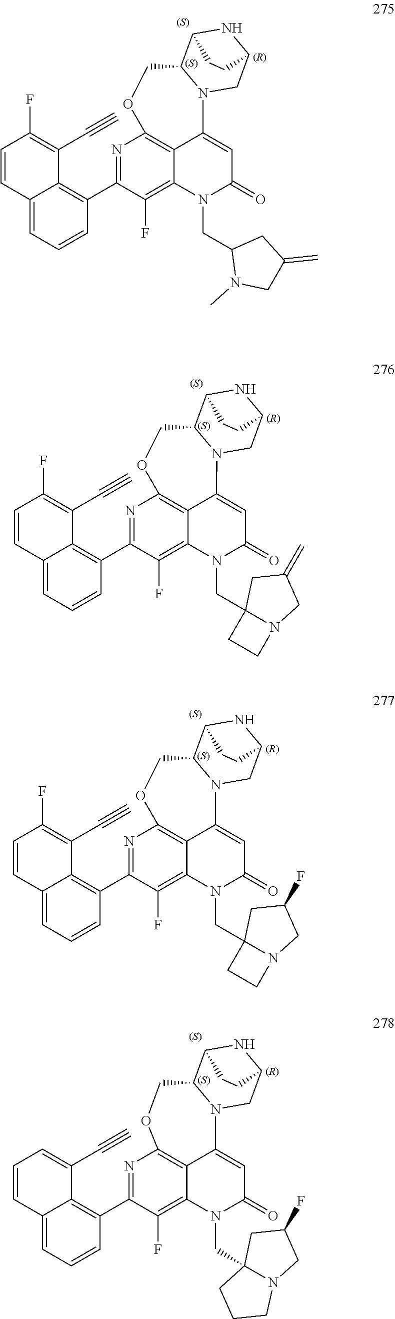 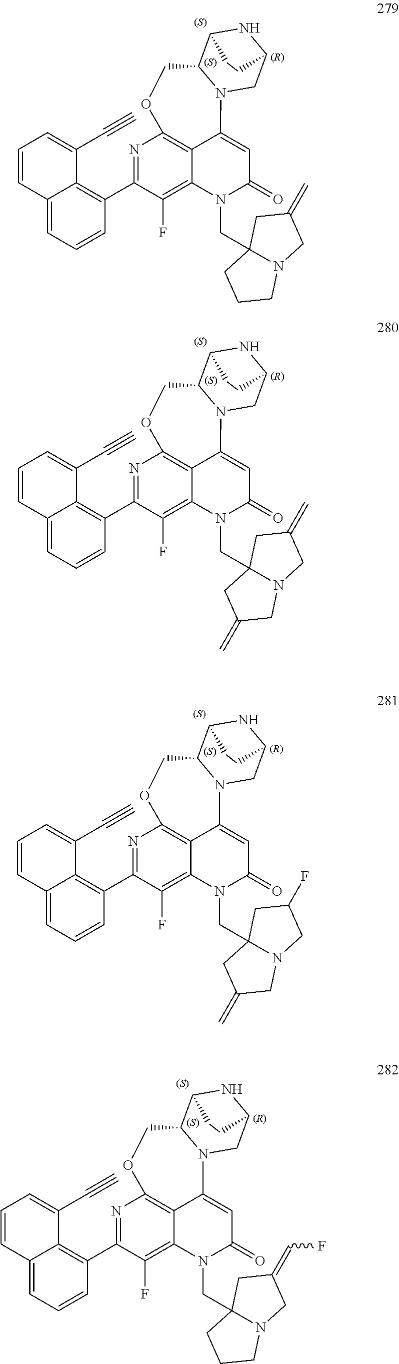 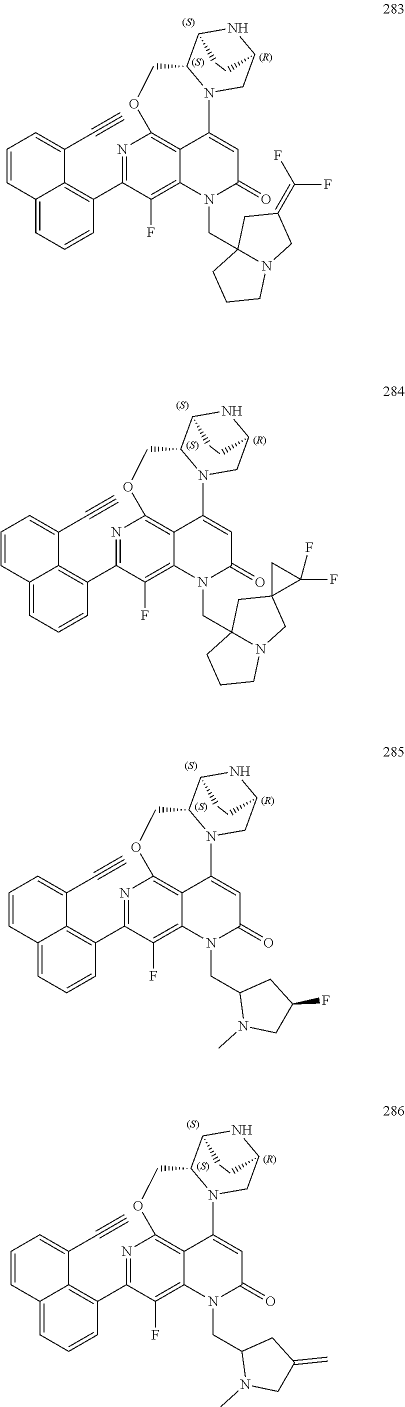 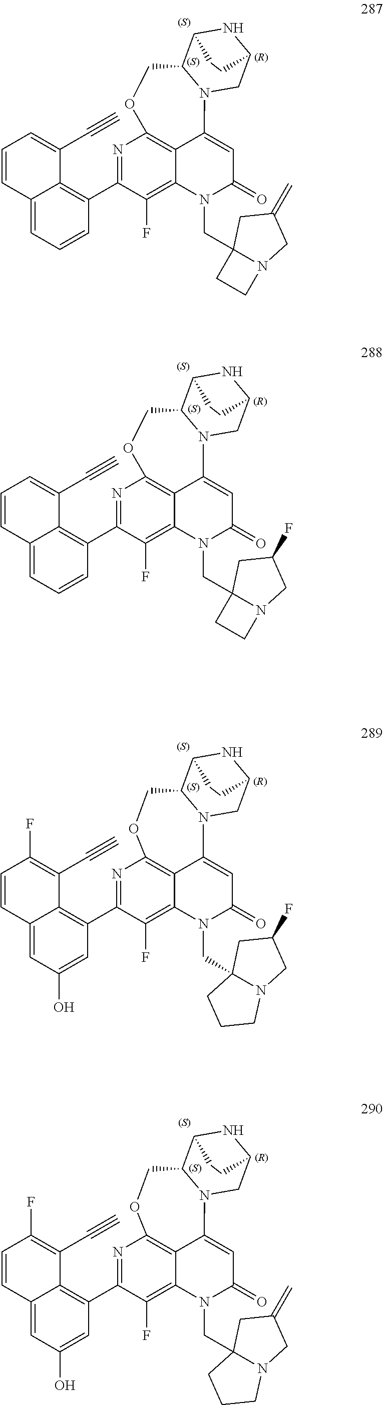 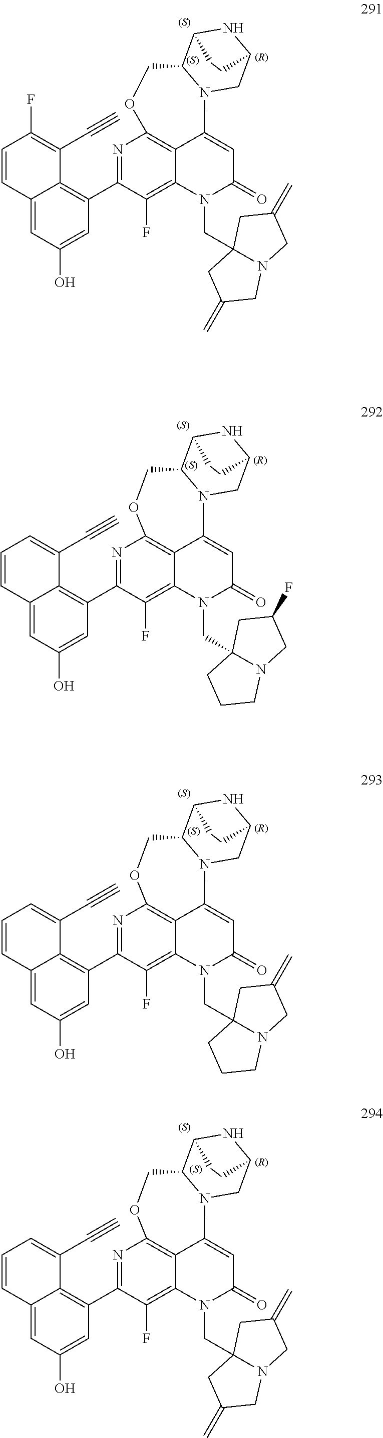 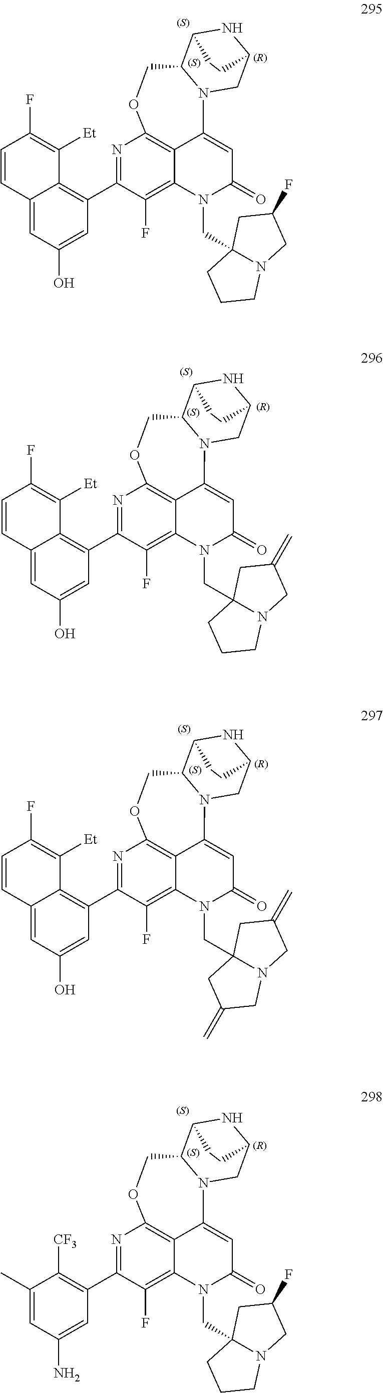 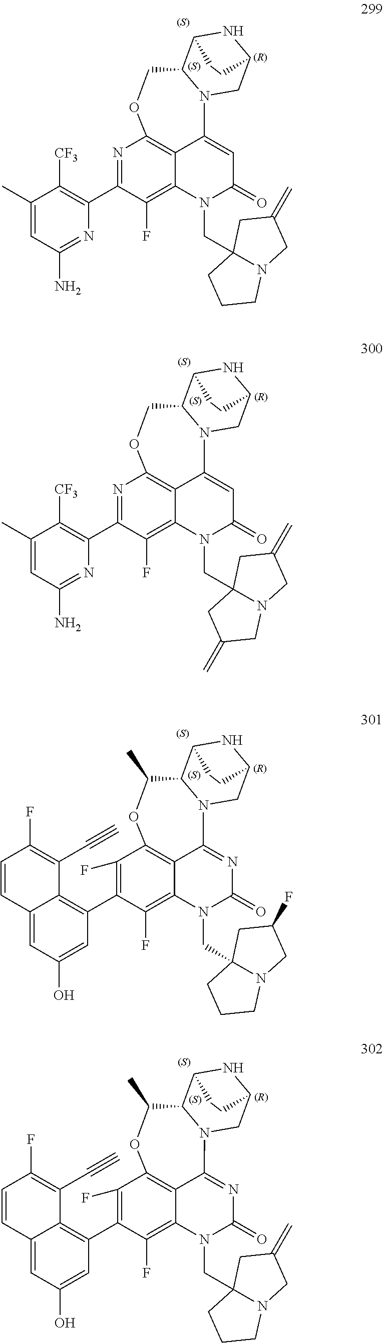 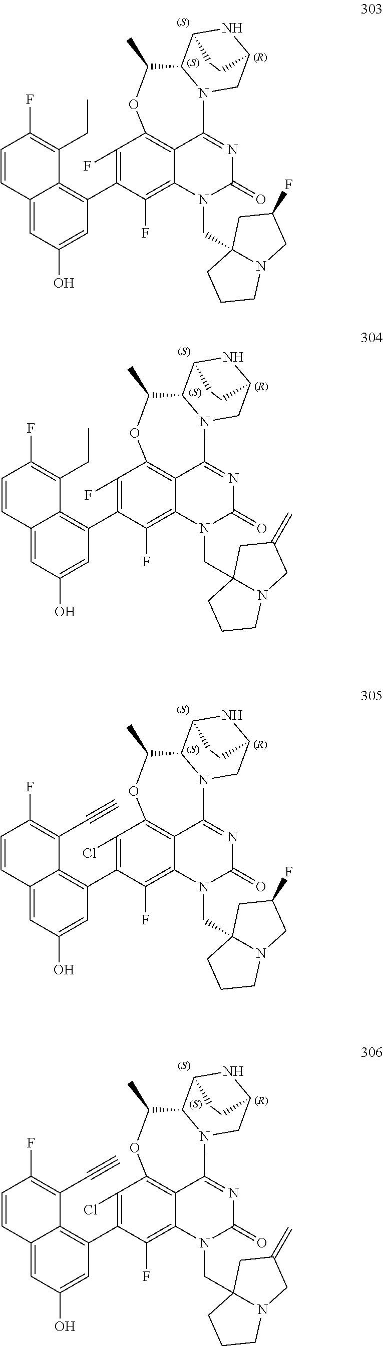 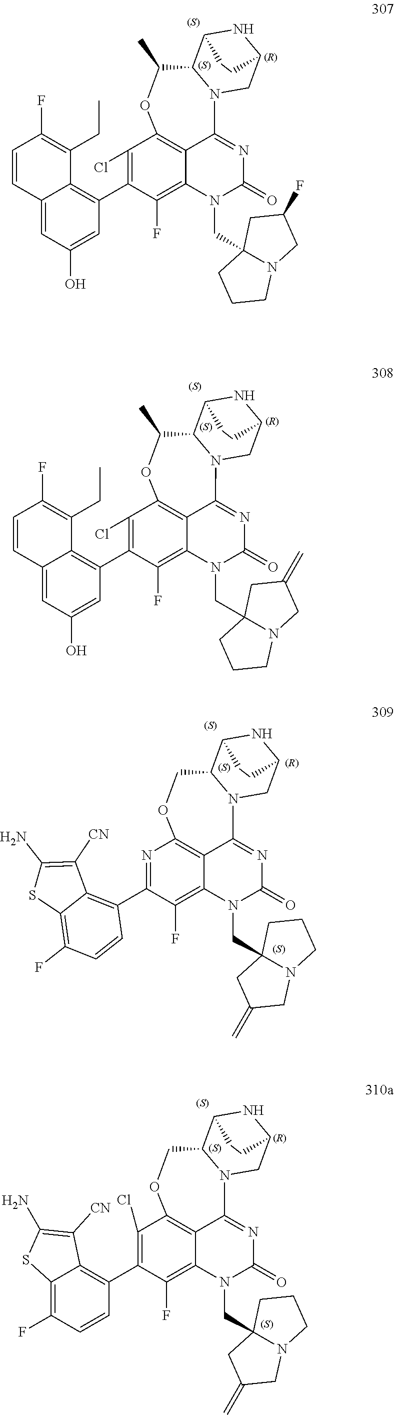 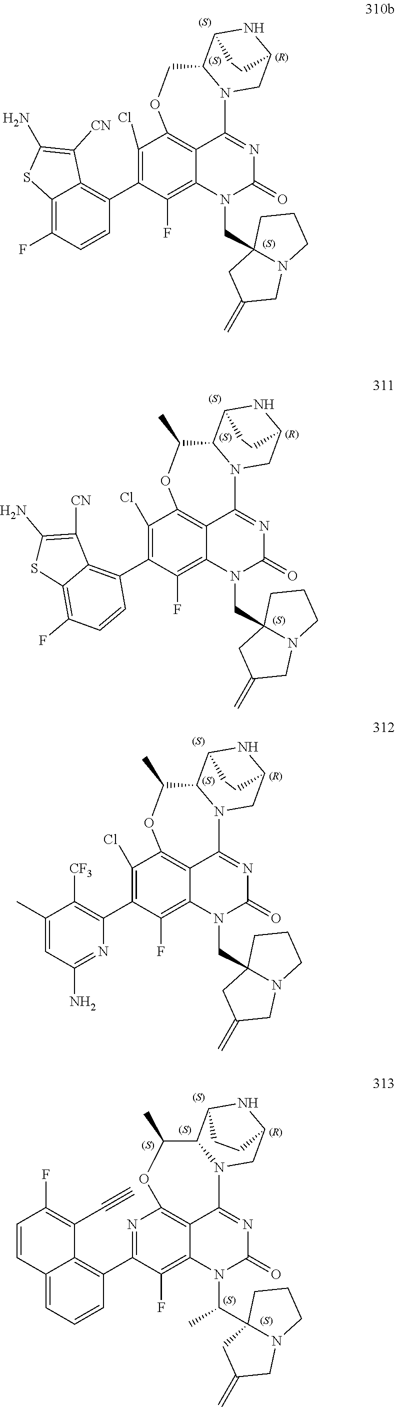 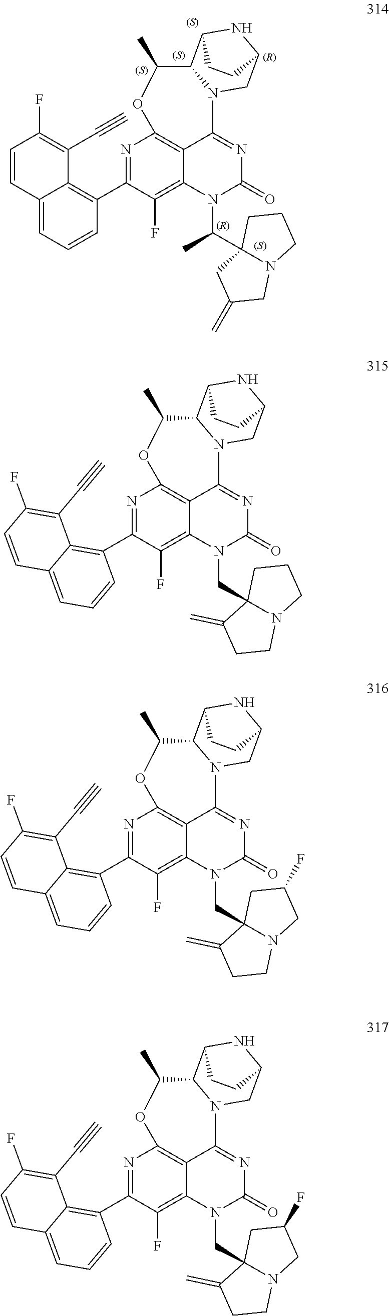 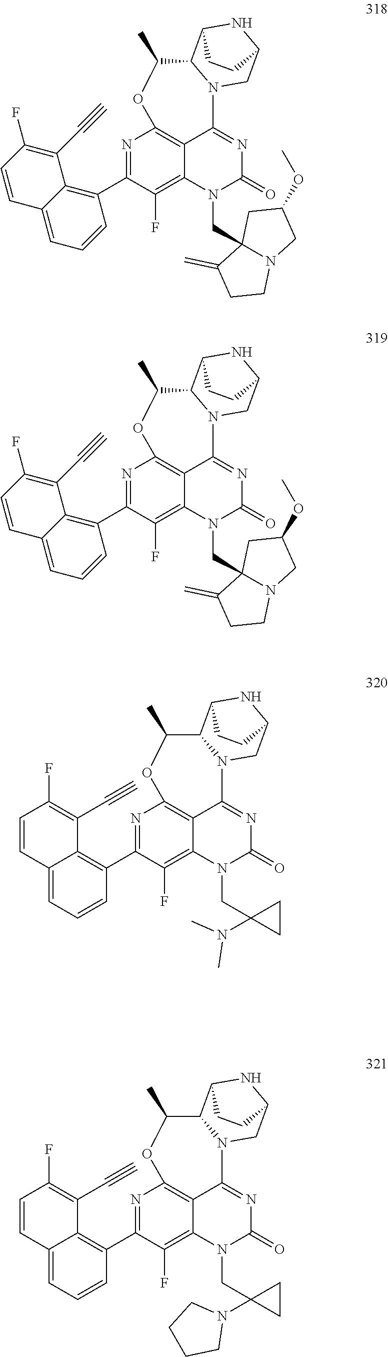 [SMILES: C#Cc1c(F)ccc2cccc(-c3nc4c5c(nc(=O)n(C[C@@]67C[C@H](OC)CN6CCC7=C)c5c3F)N3C[C@H]5CC[C@H](N5)[C@H]3[C@H](C)O4)c12 ; C#Cc1c(F)ccc2cccc(-c3nc4c5c(nc(=O)n(C[C@@]67C[C@@H](OC)CN6CCC7=C)c5c3F)N3C[C@H]5CC[C@H](N5)[C@H]3[C@H](C)O4)c12 ; C#Cc1c(F)ccc2cccc(-c3nc4c5c(nc(=O)n(CC6(N(C)C)CC6)c5c3F)N3C[C@H]5CC[C@H](N5)[C@H]3[C@H](C)O4)c12 ; C#Cc1c(F)ccc2cccc(-c3nc4c5c(nc(=O)n(CC6(N7CCCC7)CC6)c5c3F)N3C[C@H]5CC[C@H](N5)[C@H]3[C@H](C)O4)c12] 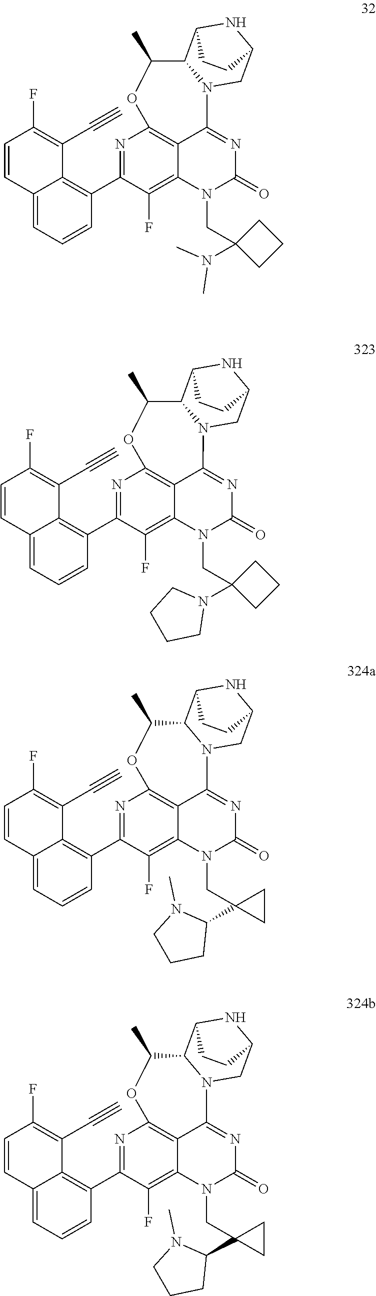 [SMILES: C#Cc1c(F)ccc2cccc(-c3nc4c5c(nc(=O)n(CC6(N(C)C)CCC6)c5c3F)N3C[C@H]5CC[C@H](N5)[C@H]3[C@H](C)O4)c12 ; C#Cc1c(F)ccc2cccc(-c3nc4c5c(nc(=O)n(CC6(N7CCCC7)CCC6)c5c3F)N3C[C@H]5CC[C@H](N5)[C@H]3[C@H](C)O4)c12 ; C#Cc1c(F)ccc2cccc(-c3nc4c5c(nc(=O)n(CC6([C@@H]7CCCN7C)CC6)c5c3F)N3C[C@H]5CC[C@H](N5)[C@H]3[C@H](C)O4)c12 ; C#Cc1c(F)ccc2cccc(-c3nc4c5c(nc(=O)n(CC6([C@H]7CCCN7C)CC6)c5c3F)N3C[C@H]5CC[C@H](N5)[C@H]3[C@H](C)O4)c12] 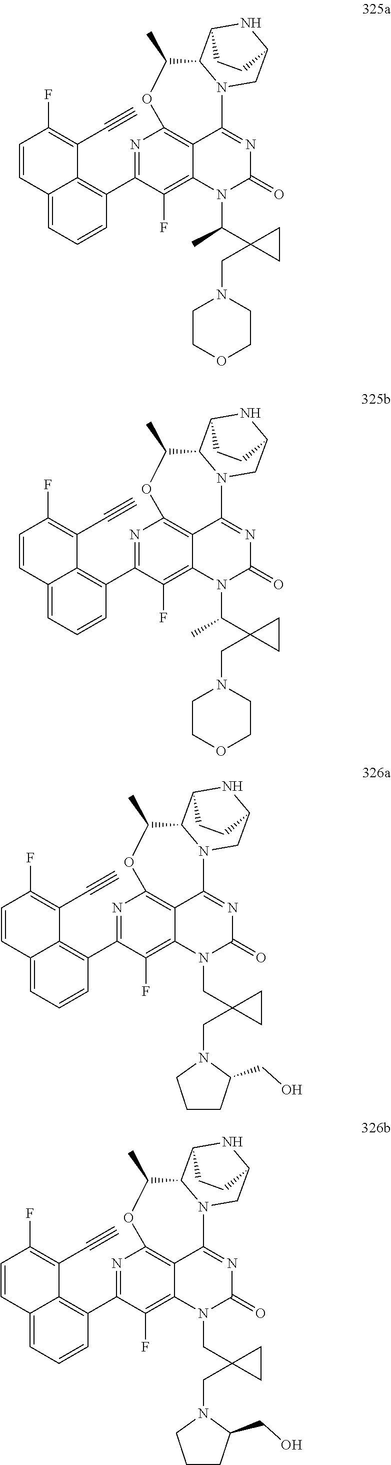 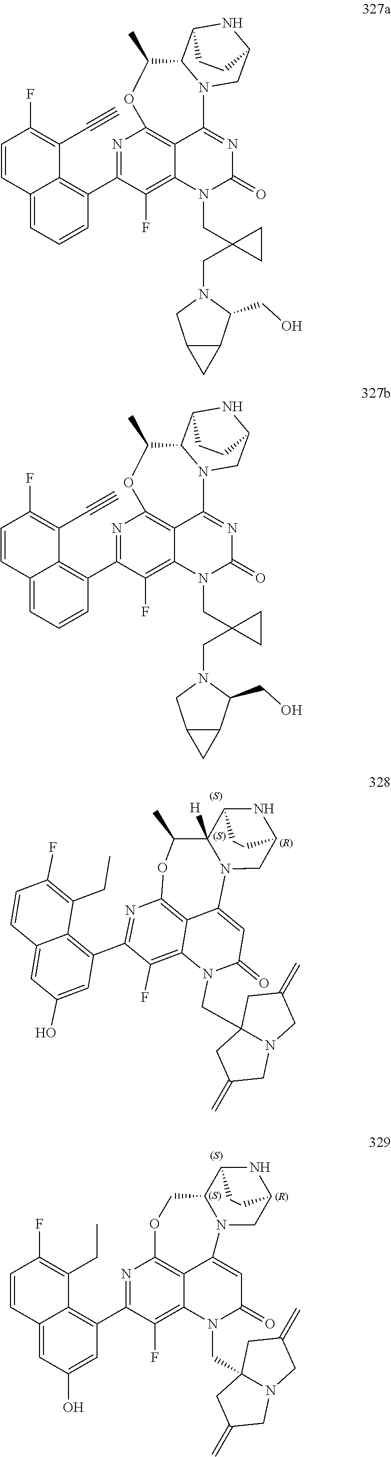 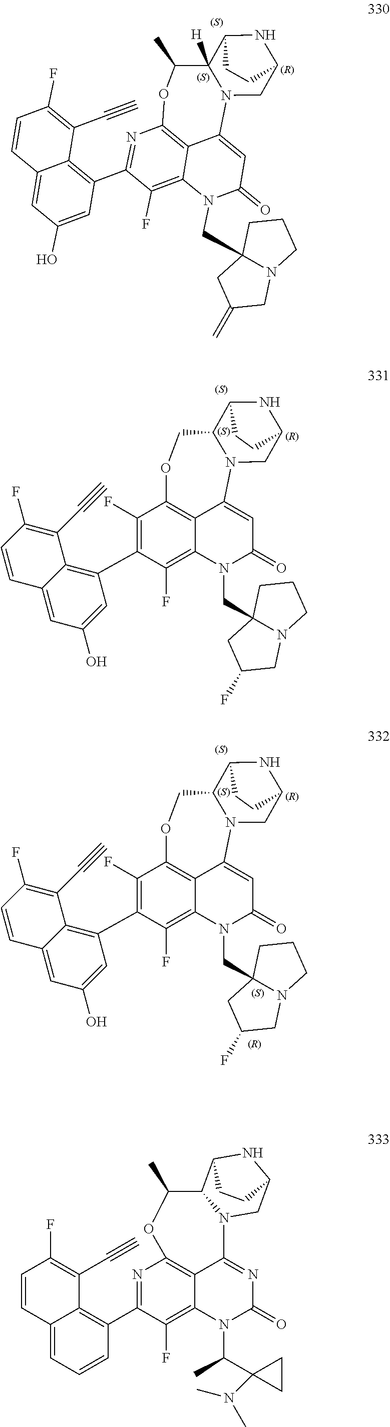 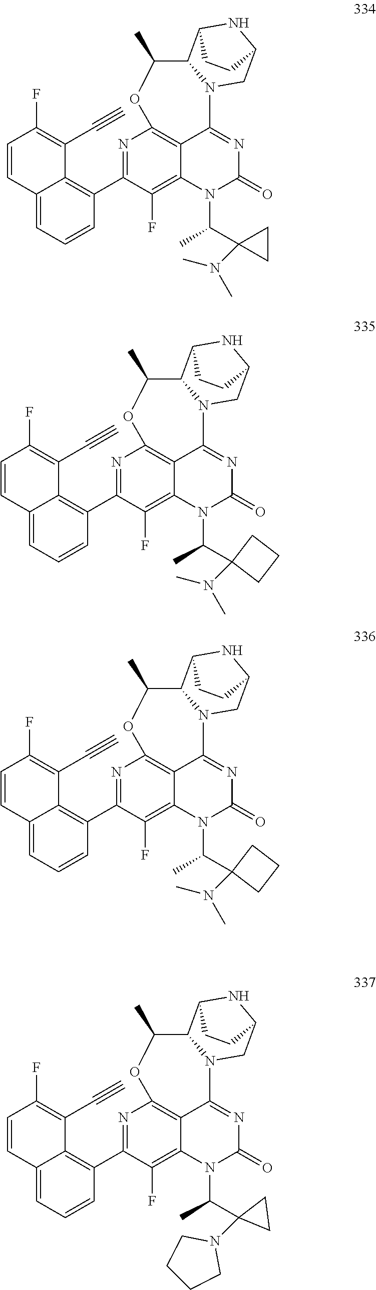 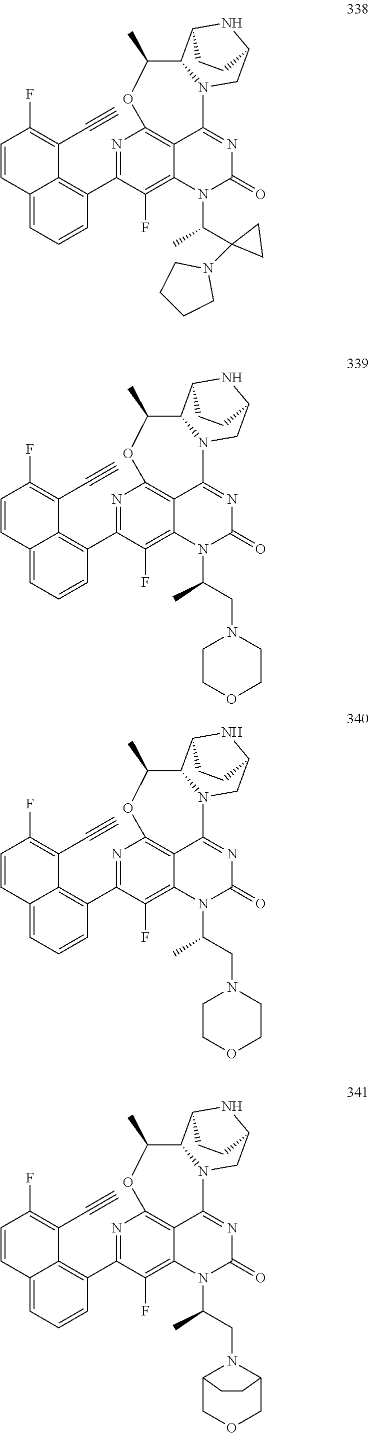 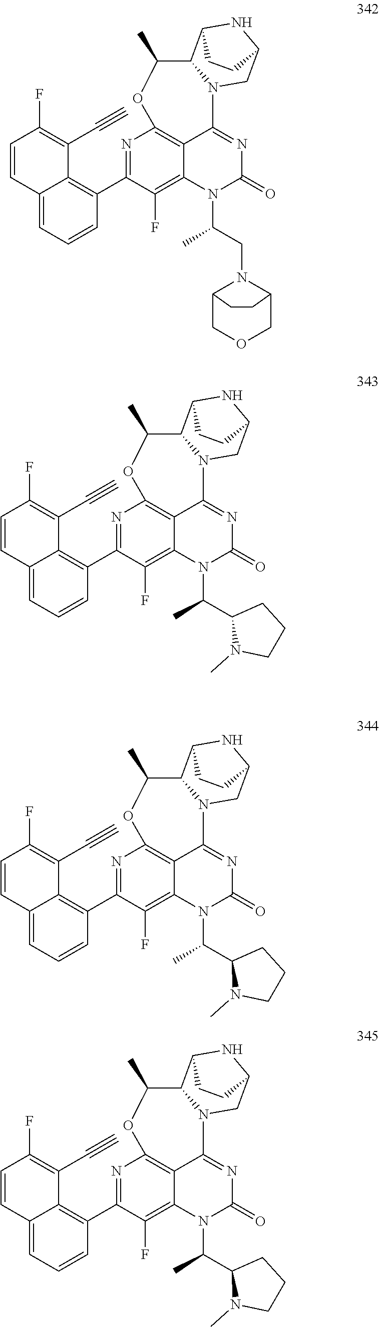 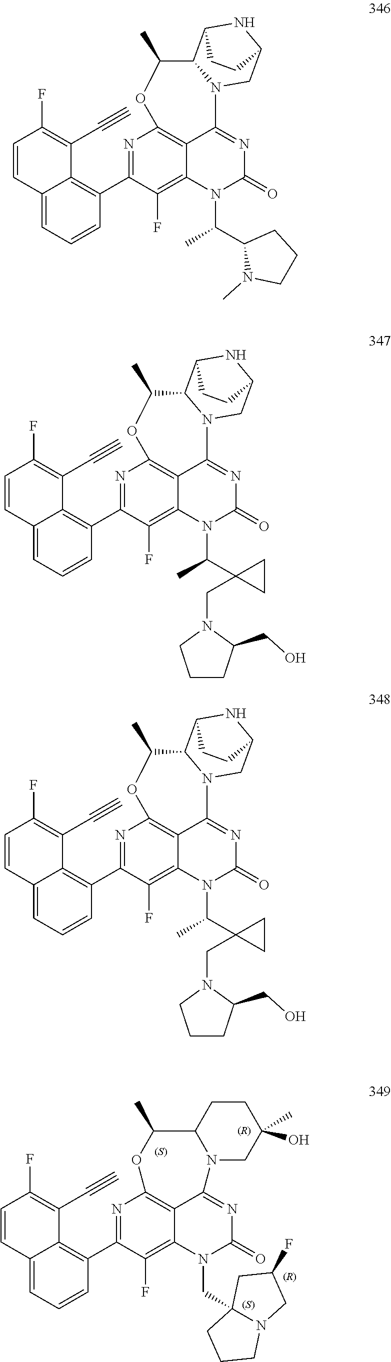 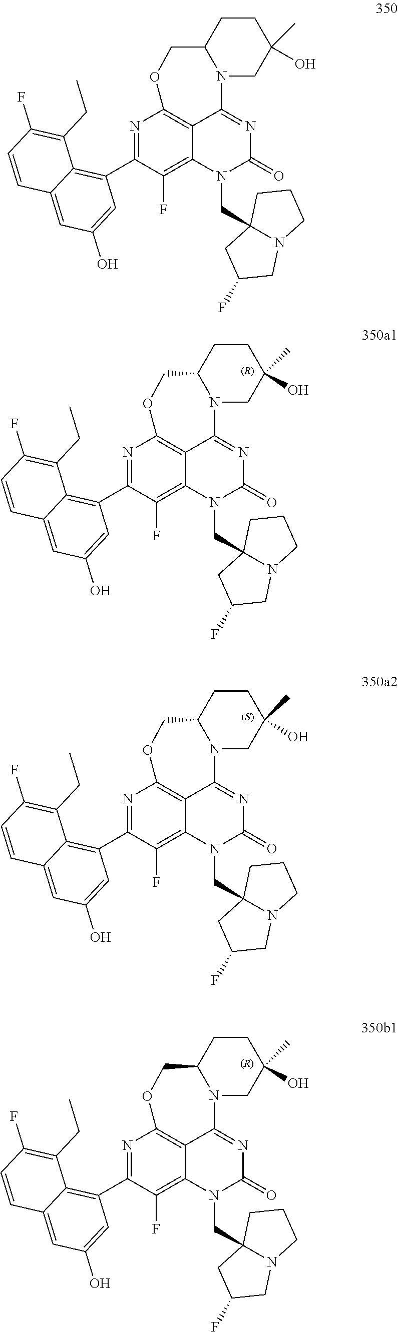 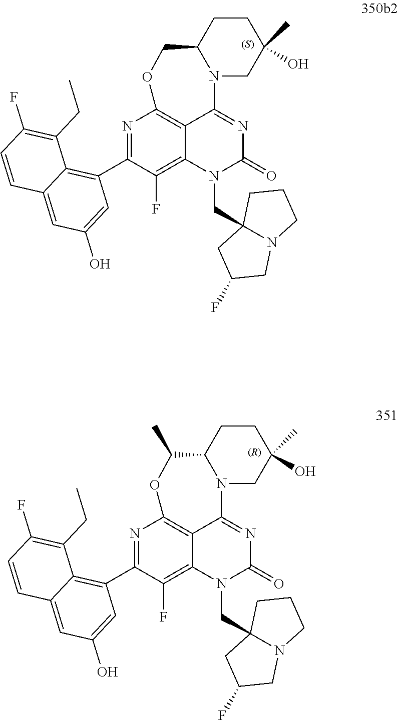 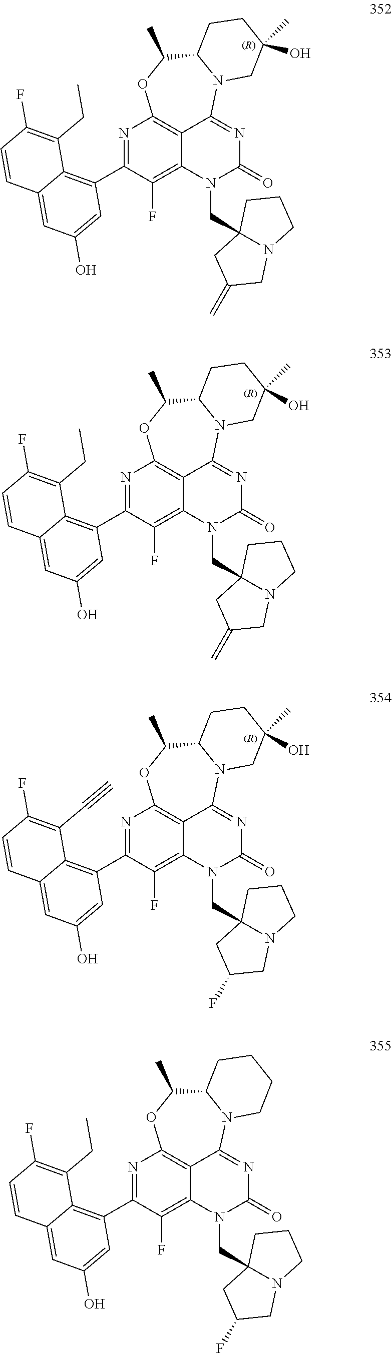 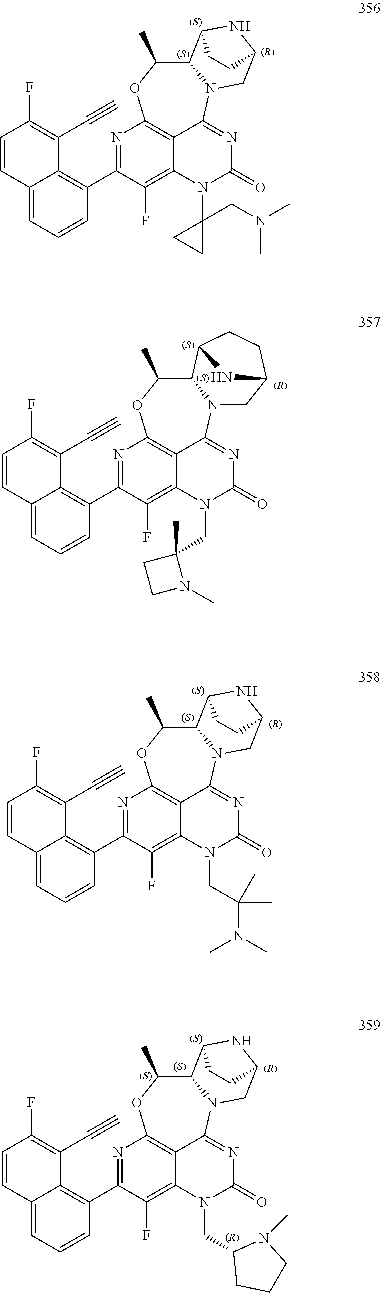 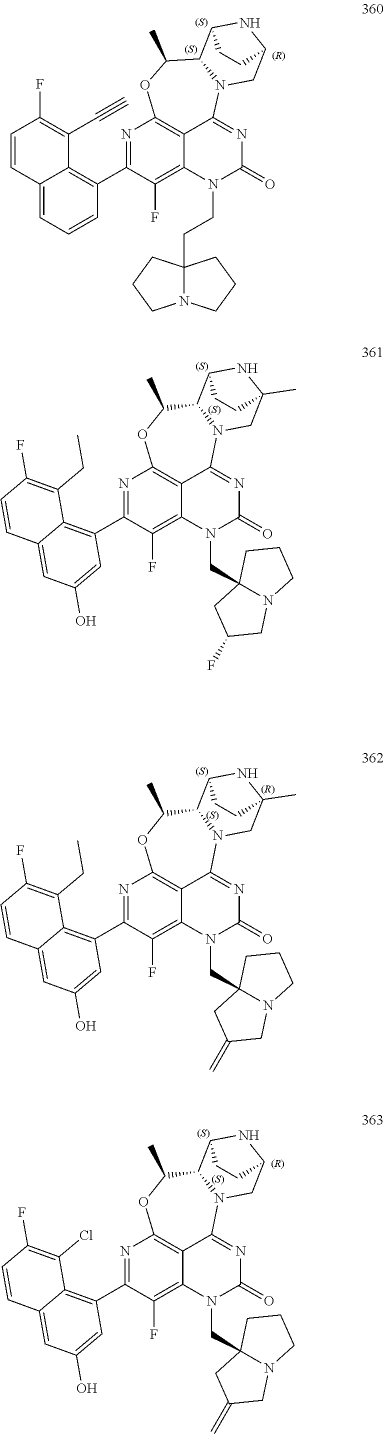 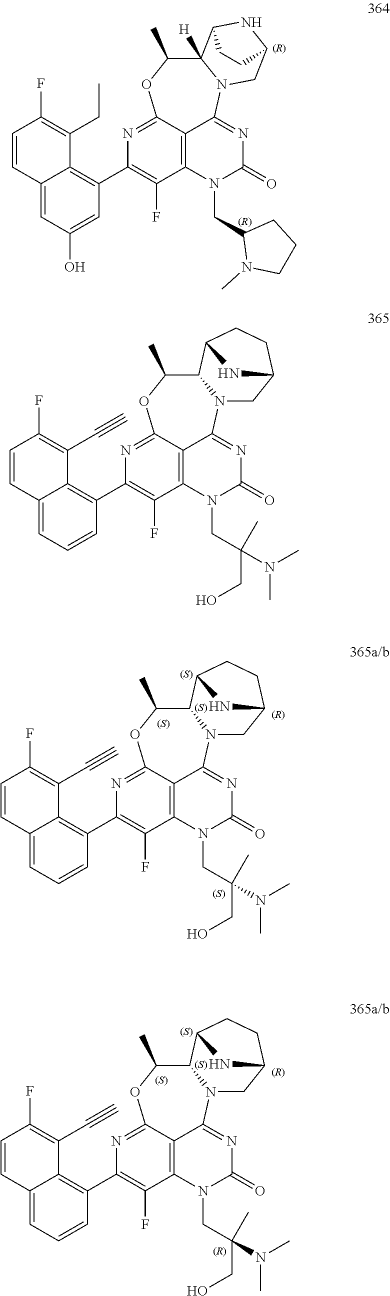 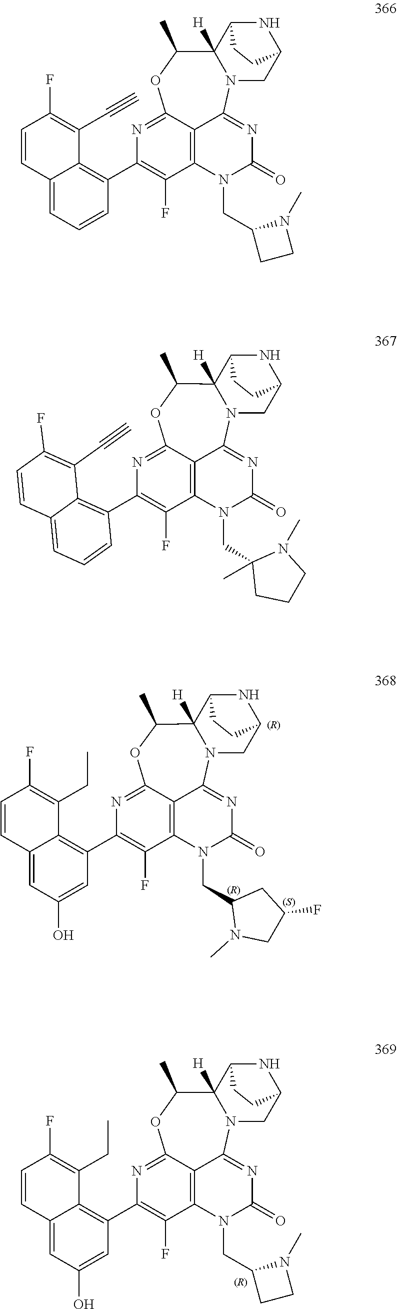 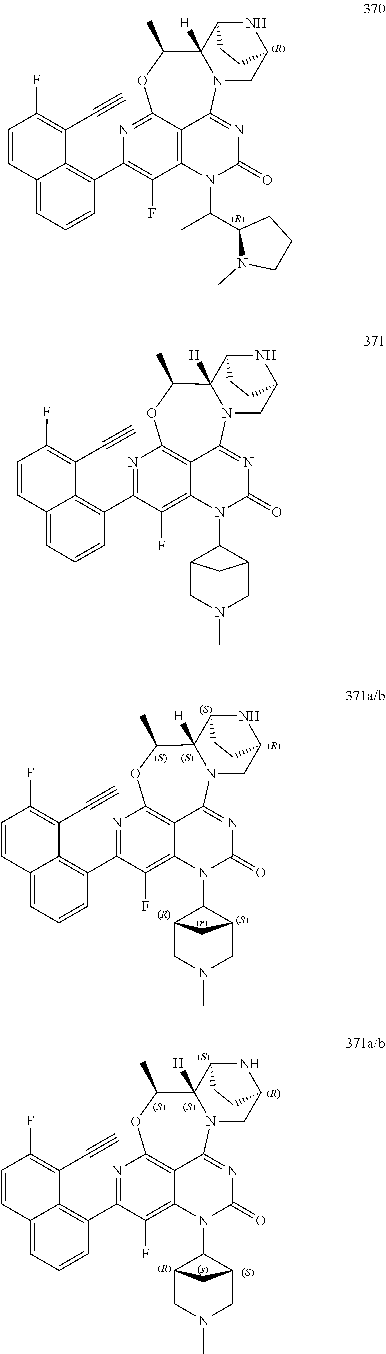 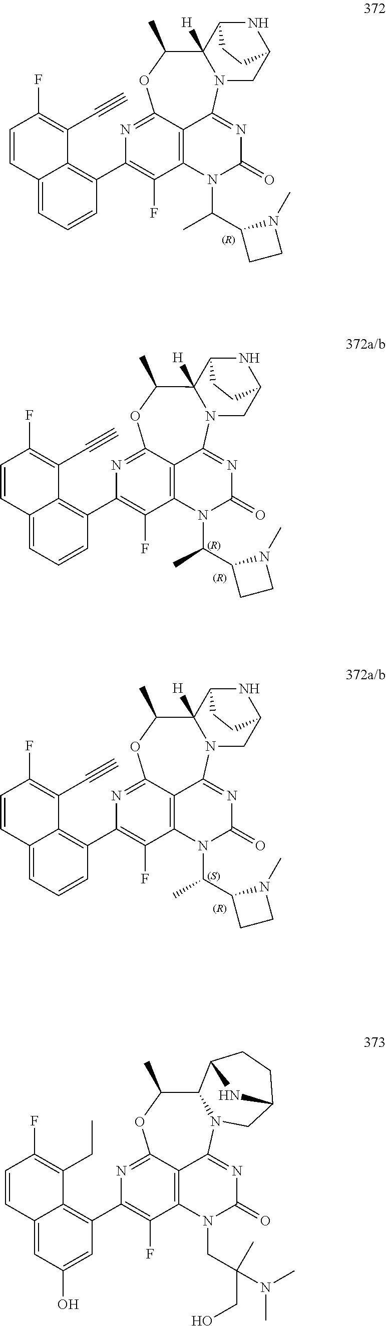 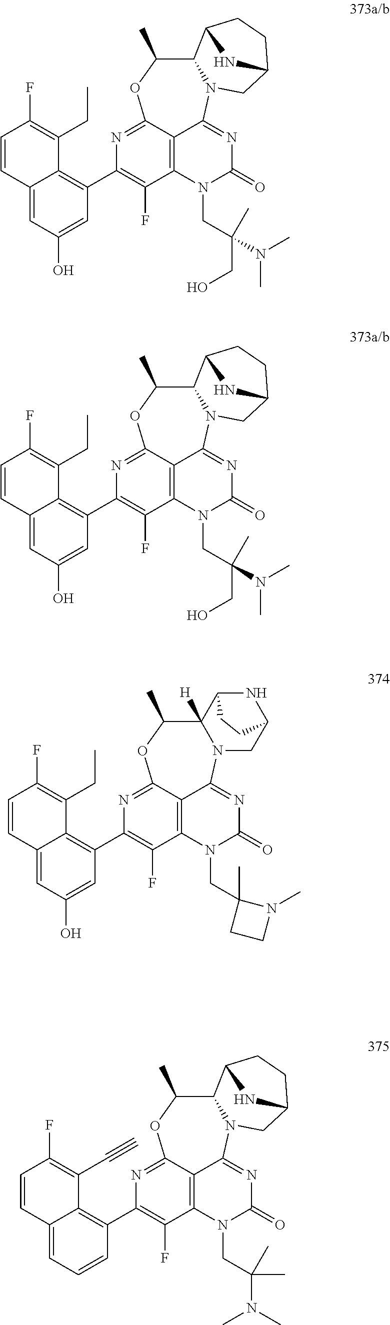 [SMILES: CCc1c(F)ccc2cc(O)cc(-c3nc4c5c(nc(=O)n(C[C@@](C)(CO)N(C)C)c5c3F)N3C[C@H]5CC[C@H](N5)[C@H]3[C@H](C)O4)c12 ; CCc1c(F)ccc2cc(O)cc(-c3nc4c5c(nc(=O)n(C[C@](C)(CO)N(C)C)c5c3F)N3C[C@H]5CC[C@H](N5)[C@H]3[C@H](C)O4)c12 ; CCc1c(F)ccc2cc(O)cc(-c3nc4c5c(nc(=O)n(CC6(C)CCN6C)c5c3F)N3C[C@H]5CC[C@H](N5)[C@H]3[C@H](C)O4)c12 ; C#Cc1c(F)ccc2cccc(-c3nc4c5c(nc(=O)n(CC(C)(C)N(C)C)c5c3F)N3C[C@H]5CC[C@H](N5)[C@H]3[C@H](C)O4)c12] 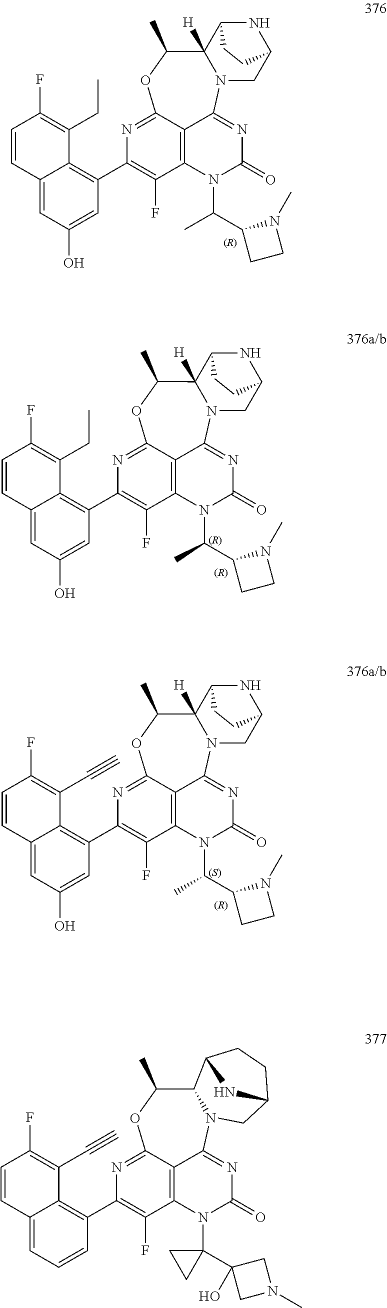 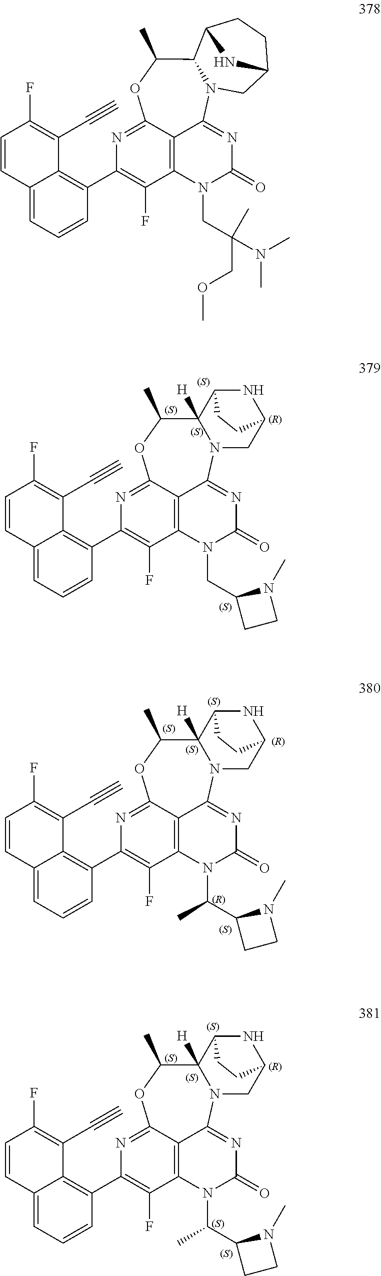 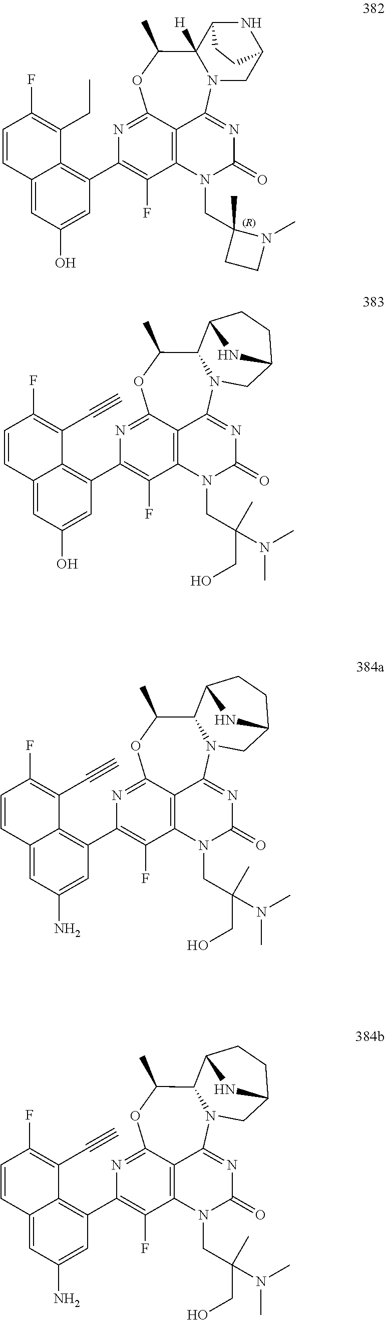 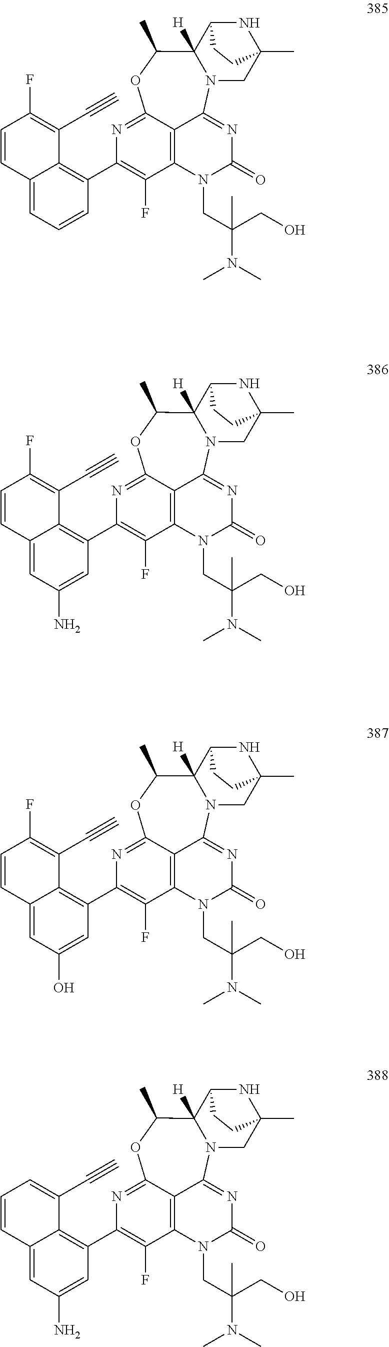 [SMILES: C#Cc1c(F)ccc2cccc(-c3nc4c5c(nc(=O)n(CC(C)(CO)N(C)C)c5c3F)N3C[C@@]5(C)CC[C@H](N5)[C@H]3[C@H](C)O4)c12 ; C#Cc1c(F)ccc2cc(N)cc(-c3nc4c5c(nc(=O)n(CC(C)(CO)N(C)C)c5c3F)N3C[C@@]5(C)CC[C@H](N5)[C@H]3[C@H](C)O4)c12 ; C#Cc1c(F)ccc2cc(O)cc(-c3nc4c5c(nc(=O)n(CC(C)(CO)N(C)C)c5c3F)N3C[C@@]5(C)CC[C@H](N5)[C@H]3[C@H](C)O4)c12 ; C#Cc1cccc2cc(N)cc(-c3nc4c5c(nc(=O)n(CC(C)(CO)N(C)C)c5c3F)N3C[C@@]5(C)CC[C@H](N5)[C@H]3[C@H](C)O4)c12] 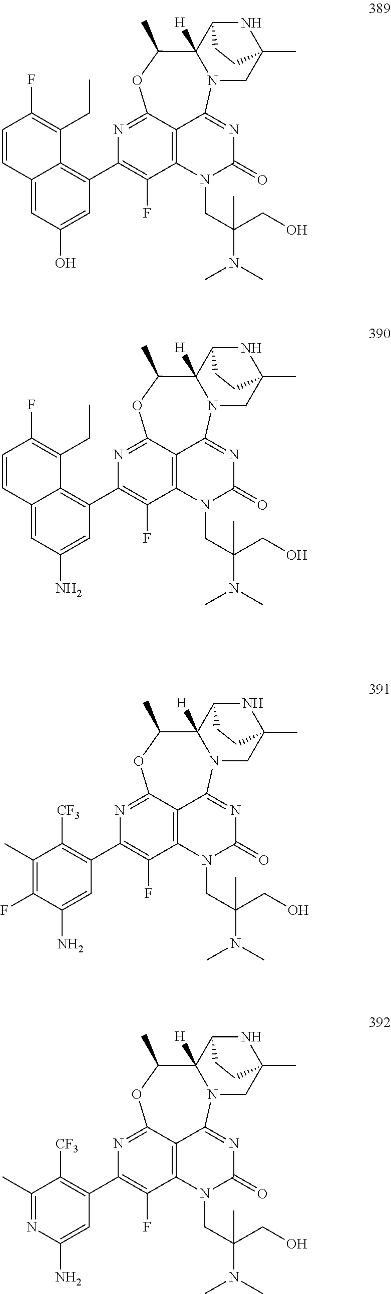 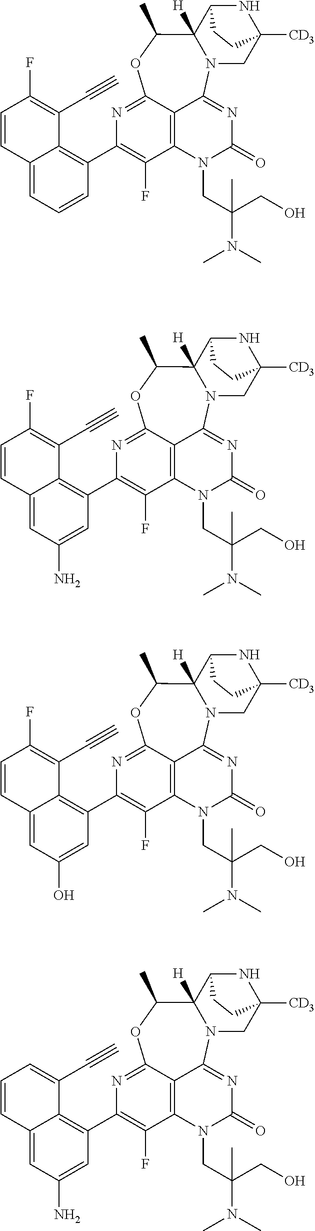 [SMILES: [2H]C([2H])([2H])[C@]12CC[C@H](N1)[C@H]1[C@H](C)Oc3nc(-c4cccc5ccc(F)c(C#C)c45)c(F)c4c3c(nc(=O)n4CC(C)(CO)N(C)C)N1C2 ; [2H]C([2H])([2H])[C@]12CC[C@H](N1)[C@H]1[C@H](C)Oc3nc(-c4cc(N)cc5ccc(F)c(C#C)c45)c(F)c4c3c(nc(=O)n4CC(C)(CO)N(C)C)N1C2 ; [2H]C([2H])([2H])[C@]12CC[C@H](N1)[C@H]1[C@H](C)Oc3nc(-c4cc(O)cc5ccc(F)c(C#C)c45)c(F)c4c3c(nc(=O)n4CC(C)(CO)N(C)C)N1C2 ; [2H]C([2H])([2H])[C@]12CC[C@H](N1)[C@H]1[C@H](C)Oc3nc(-c4cc(N)cc5cccc(C#C)c45)c(F)c4c3c(nc(=O)n4CC(C)(CO)N(C)C)N1C2] 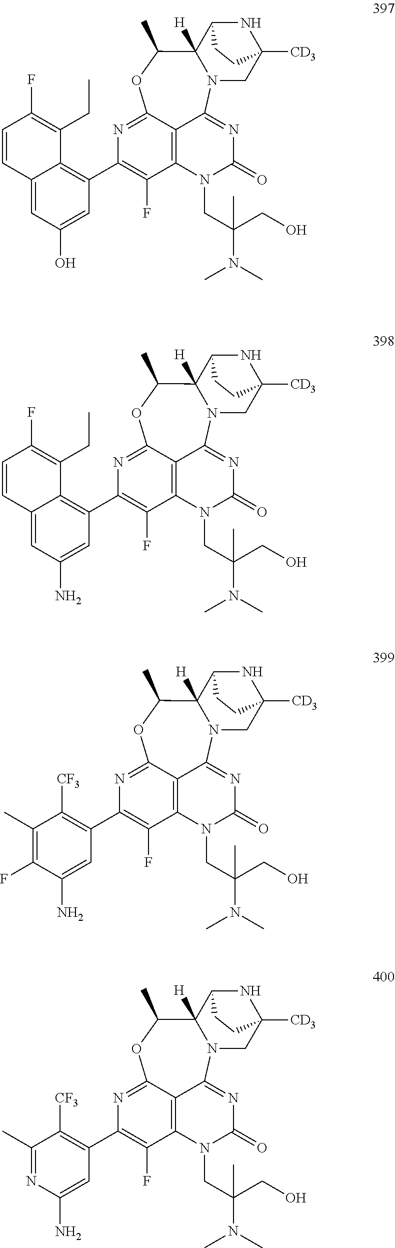 [SMILES: [2H]C([2H])([2H])[C@]12CC[C@H](N1)[C@H]1[C@H](C)Oc3nc(-c4cc(O)cc5ccc(F)c(CC)c45)c(F)c4c3c(nc(=O)n4CC(C)(CO)N(C)C)N1C2 ; [2H]C([2H])([2H])[C@]12CC[C@H](N1)[C@H]1[C@H](C)Oc3nc(-c4cc(N)cc5ccc(F)c(CC)c45)c(F)c4c3c(nc(=O)n4CC(C)(CO)N(C)C)N1C2 ; [2H]C([2H])([2H])[C@]12CC[C@H](N1)[C@H]1[C@H](C)Oc3nc(-c4cc(N)c(F)c(C)c4C(F)(F)F)c(F)c4c3c(nc(=O)n4CC(C)(CO)N(C)C)N1C2 ; [2H]C([2H])([2H])[C@]12CC[C@H](N1)[C@H]1[C@H](C)Oc3nc(-c4cc(N)nc(C)c4C(F)(F)F)c(F)c4c3c(nc(=O)n4CC(C)(CO)N(C)C)N1C2] 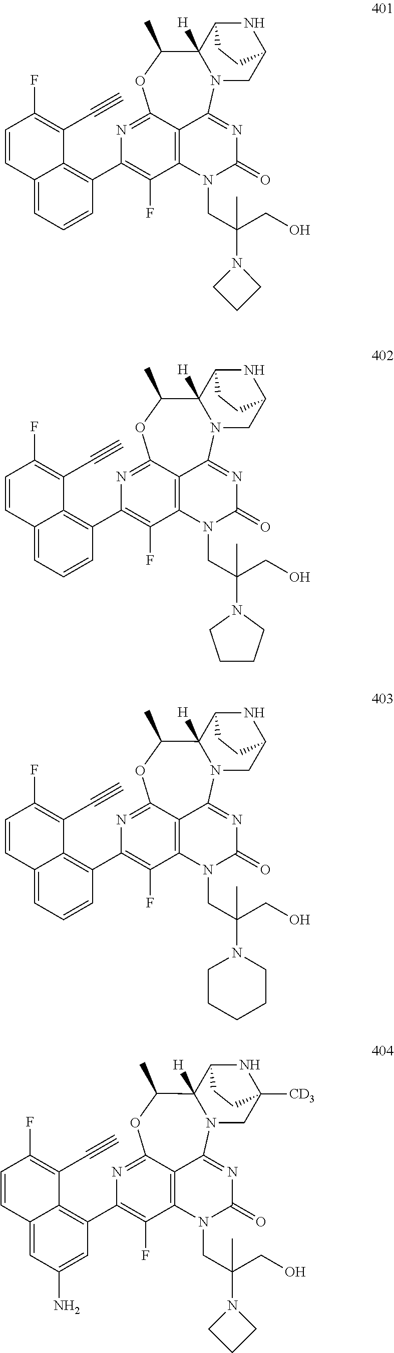 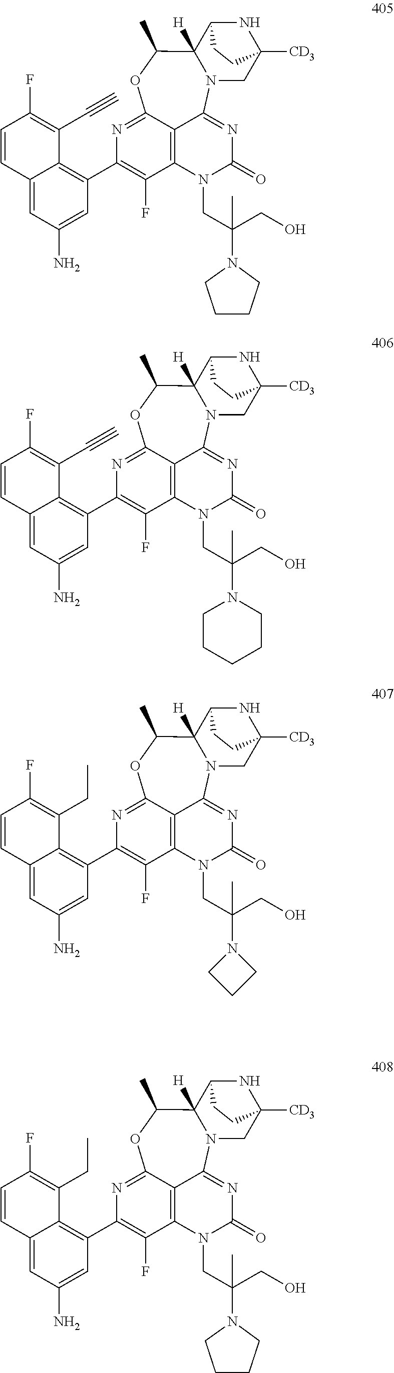 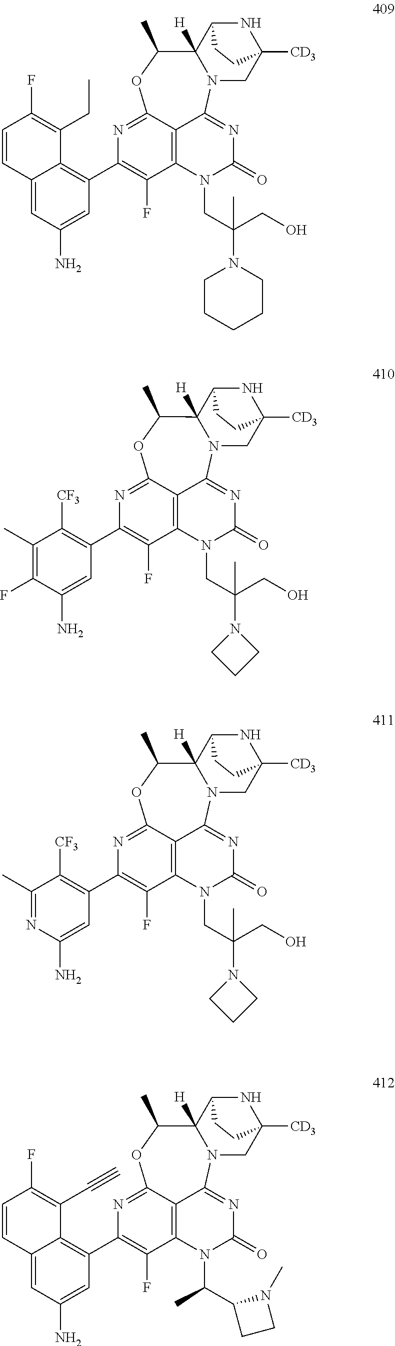 [SMILES: [2H]C([2H])([2H])[C@]12CC[C@H](N1)[C@H]1[C@H](C)Oc3nc(-c4cc(N)cc5ccc(F)c(CC)c45)c(F)c4c3c(nc(=O)n4CC(C)(CO)N3CCCCC3)N1C2 ; [2H]C([2H])([2H])[C@]12CC[C@H](N1)[C@H]1[C@H](C)Oc3nc(-c4cc(N)c(F)c(C)c4C(F)(F)F)c(F)c4c3c(nc(=O)n4CC(C)(CO)N3CCC3)N1C2 ; [2H]C([2H])([2H])[C@]12CC[C@H](N1)[C@H]1[C@H](C)Oc3nc(-c4cc(N)nc(C)c4C(F)(F)F)c(F)c4c3c(nc(=O)n4CC(C)(CO)N3CCC3)N1C2 ; [2H]C([2H])([2H])[C@]12CC[C@H](N1)[C@H]1[C@H](C)Oc3nc(-c4cc(N)cc5ccc(F)c(C#C)c45)c(F)c4c3c(nc(=O)n4[C@H](C)[C@H]3CCN3C)N1C2] 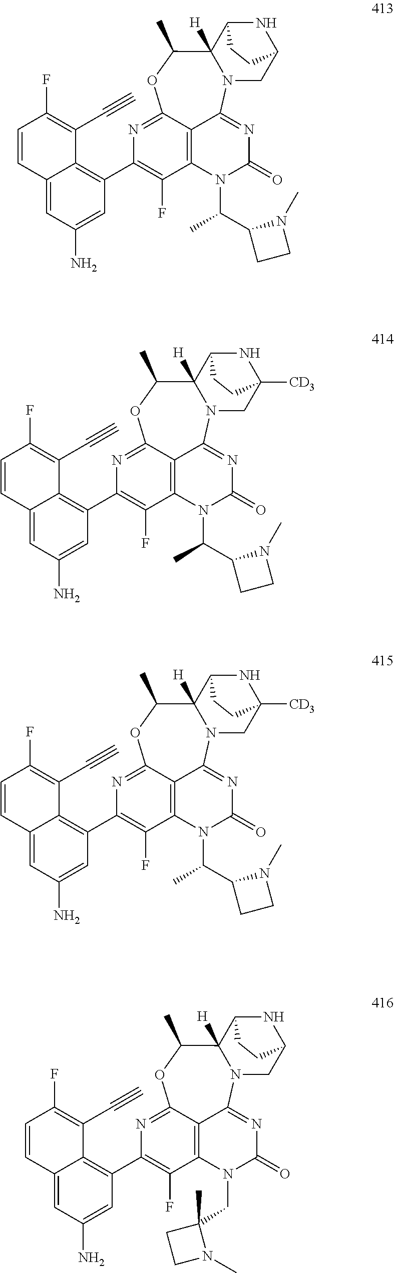 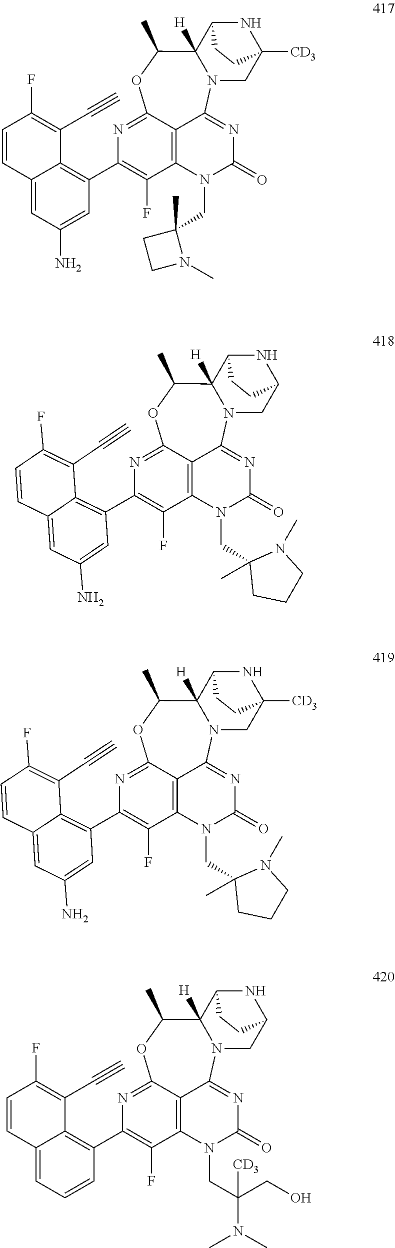 [SMILES: [2H]C([2H])([2H])[C@]12CC[C@H](N1)[C@H]1[C@H](C)Oc3nc(-c4cc(N)cc5ccc(F)c(C#C)c45)c(F)c4c3c(nc(=O)n4C[C@@]3(C)CCN3C)N1C2 ; C#Cc1c(F)ccc2cc(N)cc(-c3nc4c5c(nc(=O)n(C[C@@]6(C)CCCN6C)c5c3F)N3C[C@H]5CC[C@H](N5)[C@H]3[C@H](C)O4)c12 ; [2H]C([2H])([2H])[C@]12CC[C@H](N1)[C@H]1[C@H](C)Oc3nc(-c4cc(N)cc5ccc(F)c(C#C)c45)c(F)c4c3c(nc(=O)n4C[C@@]3(C)CCCN3C)N1C2 ; [2H]C([2H])([2H])C(CO)(Cn1c(=O)nc2c3c(nc(-c4cccc5ccc(F)c(C#C)c45)c(F)c31)O[C@@H](C)[C@@H]1[C@@H]3CC[C@H](CN21)N3)N(C)C] 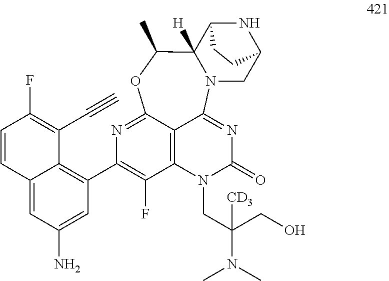 [SMILES: [2H]C([2H])([2H])C(CO)(Cn1c(=O)nc2c3c(nc(-c4cc(N)cc5ccc(F)c(C#C)c45)c(F)c31)O[C@@H](C)[C@@H]1[C@@H]3CC[C@H](CN21)N3)N(C)C] 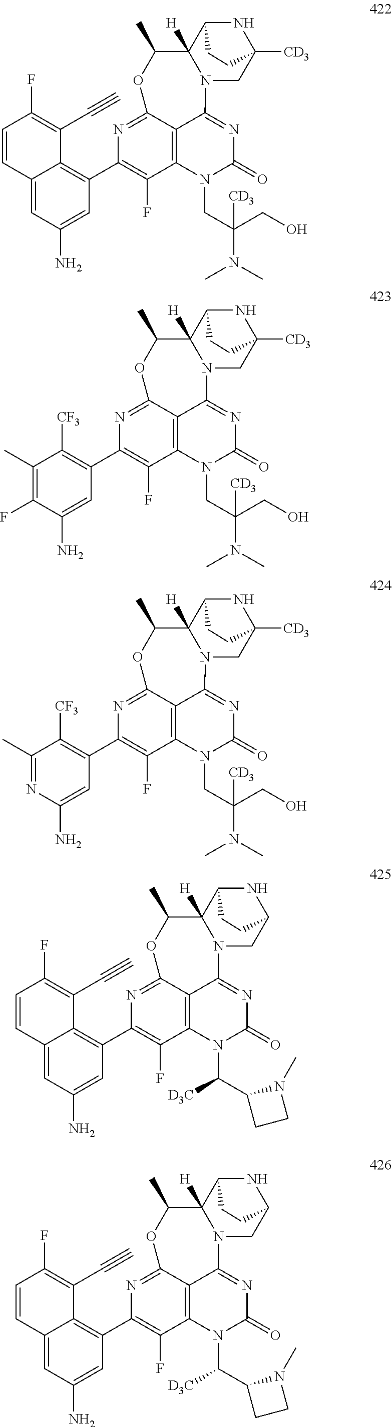 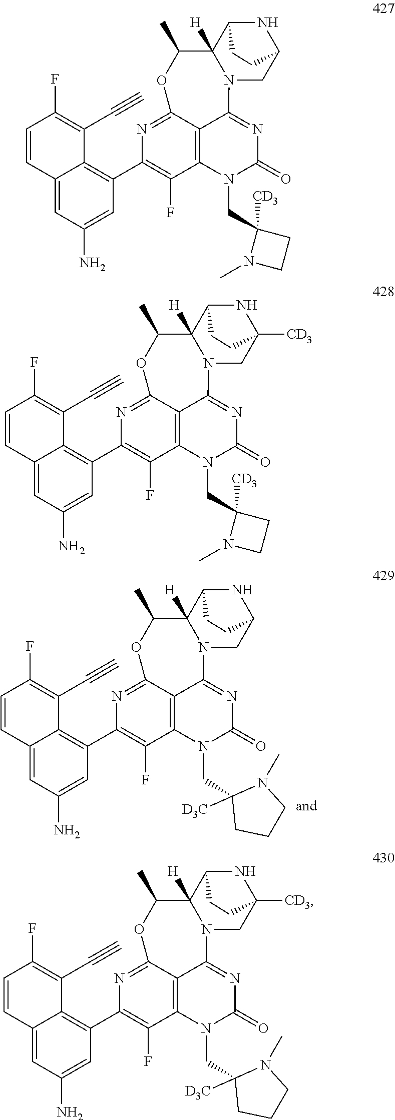
or a pharmaceutically acceptable salt thereof.

94. A pharmaceutical composition comprising the compound of any one of claims 1-93 or a pharmaceutically acceptable salt thereof, and a pharmaceutically acceptable carrier.

95. A method for inhibiting KRas G12D activity in a subject in need thereof, comprising administering an effective amount of a compound of any one of claims 1-93 or a pharmaceutically acceptable salt thereof or the pharmaceutical composition of claim 83 to the subject.

96. A method for treating a KRas G12D-associated cancer comprising administering an effective amount of a compound of any one of claims 1-93 or a pharmaceutically acceptable salt thereof or the pharmaceutical composition of claim 94 to a subject in need thereof.

97. The method of claim 96, wherein the KRas G12D-associated cancer is selected from the group consisting of:
(i) Cardiac: sarcoma (angiosarcoma, fibrosarcoma, rhabdomyosarcoma, liposarcoma), myxoma, rhabdomyoma, fibroma, lipoma and teratoma;
(ii) Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma;
(iii) Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma);
(iv) Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma);
(v) Liver: hepatoma (hepatocellular carcinoma), cholangiocarcinoma, hepatoblastoma, angiosarcoma, hepatocellular adenoma, hemangioma;
(vi) Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors;
(vii) Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma);
(viii) Gynecological: uterus (endometrial carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma);
(ix) Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma);
(x) Skin: malignant melanoma, basal cell carcinoma, squamous cell carcinoma, Kaposi's sarcoma, moles dysplastic nevi, lipoma, angioma, dermatofibroma, keloids, psoriasis; and
(xi) Adrenal glands: neuroblastoma.

98. The method of claim 97, wherein the cancer is non-small cell lung cancer, small cell lung cancer, colorectal cancer, rectal cancer or pancreatic cancer.

99. A method for treating cancer in a subject in need thereof, the method comprising (a) acquiring the knowledge that the cancer is associated with a KRas G12D mutation; and (b) administering to the subject an effective amount of a compound of any one of claims 1-93 or a pharmaceutically acceptable salt thereof or the pharmaceutical composition of claim 94.

100. The method of any one of claims 95-99, wherein the administering is conducted via a route selected from the group consisting of parenteral, intraperitoneal, intradermal, intracardiac, intraventricular, intracranial, intracerebrospinal, intrasynovial, intrathecal administration, intramuscular injection, intravitreous injection, intravenous injection, intra-arterial injection, oral, buccal, sublingual, transdermal, topical, intratracheal, intrarectal, subcutaneous, and topical administration.

101. The method of any one of claims 95-99, wherein the compound is administered simultaneously, separately or sequentially with one or more additional therapeutic agents.

102. The method of claim 101, wherein the one or more additional therapeutic agents are selected from an anti-PD-1 or PD-L1 antagonist, an MEK inhibitor, a CDK4/CDK6 inhibitor, an EGFR inhibitor, ERK inhibitor, a SHP2 inhibitor, a platinum agent or pemetrexed.

103. Use of the compound of any one of claims 1-93 or a pharmaceutically acceptable salt thereof or the pharmaceutical composition of claim 94 in the manufacture of a medicament for treating cancer.

104. A compound of any one of claims 1-93 or a pharmaceutically acceptable salt thereof or the pharmaceutical composition of claim 94, for use in the treatment of cancer.
